SEQUENCE LISTING (1116 sequences)

SEQ ID NO: 1 - protein, 1427 aa, Burkholderia cenocepacia
  REGION 1..1427: note = P0DUH5|DDDA_BURC1 Double-stranded DNA deaminase toxin A
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAVVRQFAYTNGLMTSHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHFDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDGAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGAPQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 2 - protein, 11 aa, synthetic construct
  REGION 1..11: note = AvrBs3 C11 consensus
SIVAQLSRPDP

SEQ ID NO: 3 - protein, 40 aa, synthetic construct
  REGION 1..40: note = 40 AA V2 RR C-terminal
  VARIANT 37..38: note = misc_feature - Xaa can be Arg or His
SIVAQLSRPDPALAALTNDHLVALACLGGRPALDAVXXGL

SEQ ID NO: 4 - protein, 50 aa, synthetic construct
  REGION 1..50: note = 50 AA V2 RR C-terminal
  VARIANT 37..38: note = misc_feature - Xaa can be Arg or His
SIVAQLSRPDPALAALTNDHLVALACLGGRPALDAVXXGLPHAPALIXRT

SEQ ID NO: 5 - protein, 34 aa, synthetic construct
  REGION 1..34: note = AvrBs3 Repeat DD
  VARIANT 12..13: note = misc_feature - Xaa can be any naturally occuring amino acid or can be absent
LTPDQVVAIASXXGGKQALETVQRLLPVLCQDHG

SEQ ID NO: 6 - protein, 34 aa, synthetic construct
  REGION 1..34: note = AvrBs3 Repeat DD + A23
  VARIANT 12..13: note = misc_feature - Xaa can be any naturally occuring amino acid or can be absent
LTPDQVVAIASXXGGKQALETVQALLPVLCQDHG

SEQ ID NO: 7 - protein, 34 aa, synthetic construct
  REGION 1..34: note = AvrBs3 Repeat DD + Q24
  VARIANT 12..13: note = misc_feature - Xaa can be any naturally occuring amino acid or can be absent
LTPDQVVAIASXXGGKQALETVQQLLPVLCQDHG

SEQ ID NO: 8 - protein, 34 aa, synthetic construct
  REGION 1..34: note = AvrBs3 Repeat DD + L6
  VARIANT 12..13: note = misc_feature - Xaa can be any naturally occuring amino acid or can be absent
LTPDQLVAIASXXGGKQALETVQRLLPVLCQDHG

SEQ ID NO: 9 - protein, 34 aa, synthetic construct
  REGION 1..34: note = AvrBs3 Repeat DD + M6
  VARIANT 12..13: note = misc_feature - Xaa can be any naturally occuring amino acid or can be absent
LTPDQMVAIASXXGGKQALETVQRLLPVLCQDHG

SEQ ID NO: 10 - protein, 34 aa, synthetic construct
  REGION 1..34: note = AvrBs3 Repeat DD + Q34
  VARIANT 12..13: note = misc_feature - Xaa can be any naturally occuring amino acid or can be absent
LTPDQVVAIASXXGGKQALETVQRLLPVLCQDQG

SEQ ID NO: 11 - protein, 34 aa, synthetic construct
  REGION 1..34: note = AvrBs3 Repeat DD + L3
  VARIANT 12..13: note = misc_feature - Xaa can be any naturally occuring amino acid or can be absent
LTLDQVVAIASXXGGKQALETVQRLLPVLCQDHG

SEQ ID NO: 12 - protein, 34 aa, synthetic construct
  REGION 1..34: note = TALE Repeat AvrBs3 NI canonical
MTPEQVVAIASNIGGKQALETVQALLPVLCQAHG

SEQ ID NO: 13 - protein, 34 aa, synthetic construct
  REGION 1..34: note = TALE Repeat AvrBs3 HD canonical
MTPEQVVAIASHDGGKQALETVQRLLPVLCQAHG

SEQ ID NO: 14 - protein, 34 aa, synthetic construct
  REGION 1..34: note = TALE Repeat AvrBs3 NN canonical
MTPQQVVAIASNNGGKQALETVQRLLPVLCQAHG

SEQ ID NO: 15 - protein, 34 aa, synthetic construct
  REGION 1..34: note = TALE Repeat AvrBs3 NG canonical
MTPQQVVAIASNGGGKQALETVQRLLPVLCQAHG

SEQ ID NO: 16 - protein, 925 aa, synthetic construct
  REGION 1..925: note = Left TRAC TALEN monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 17 - protein, 925 aa, synthetic construct
  REGION 1..925: note = Right TRAC TALEN monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 18 - protein, 925 aa, synthetic construct
  REGION 1..925: note = Left CD52 TALEN monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 19 - protein, 925 aa, synthetic construct
  REGION 1..925: note = right CD52 TALEN monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 20 - protein, 917 aa, synthetic construct
  REGION 1..917: note = CD52 TALE-BE #1 -L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 21 - protein, 839 aa, synthetic construct
  REGION 1..839: note = CD52 TALE-BE #1 -R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 22 - protein, 917 aa, synthetic construct
  REGION 1..917: note = CD52 TALE-BE #2 -L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 23 - protein, 839 aa, synthetic construct; identical to SEQ ID NO: 21
  REGION 1..839: note = CD52 TALE-BE #2 -R

SEQ ID NO: 24 - protein, 917 aa, synthetic construct
  REGION 1..917: note = CD52 TALE-BE #3 -L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 25 - protein, 839 aa, synthetic construct; identical to SEQ ID NO: 21
  REGION 1..839: note = CD52 TALE-BE #3 -R

SEQ ID NO: 26 - protein, 917 aa, synthetic construct
  REGION 1..917: note = CD52 TALE-BE SP -L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 27 - protein, 839 aa, synthetic construct
  REGION 1..839: note = CD52 TALE-BE SP -R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 28 - protein, 1397 aa, Burkholderia cenocepacia
  REGION 1..1397: note = DddA N-half
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAVVRQFAYTNGLMTSHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHFDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDGAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGAPQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 29 - protein, 30 aa, Burkholderia cenocepacia
  REGION 1..30: note = DddA C-half
AIPVKRGATGETKVFTGNSNSPKSPTKGGC

SEQ ID NO: 30 - protein, 82 aa, Bacillus sp.
  REGION 1..82: note = canonical UGI (uracil-DNA-glycosylase inhibitor)
  source 1..82: note = Bacillus phage PBS2
NLSDIIEKETGKQLVIQESILMLPEEVEEVIGNKPESDILVHTAYDESTDENVMLLTSDAPEYKPWALVIQDSNGENKIKML

SEQ ID NO: 31 - DNA, 49 bp, Homo sapiens
  misc_feature 1..49: note = TRAC TALE-Nuclease target sequence
  source 1..49: mol_type = genomic DNA
ttcctcctactcaccatcagcctcctggttatggtacaggtaagagcaa

SEQ ID NO: 32 - DNA, 58 bp, Homo sapiens
  misc_feature 1..58: note = CD52 TALE-Nuclease target sequence
  source 1..58: mol_type = genomic DNA
ttcctcctactcaccatcagcctcctggttatggtacaggtaagagcaacgcctggca

SEQ ID NO: 33 - DNA, 49 bp, synthetic construct
  misc_feature 1..49: note = T-1
  source 1..49: mol_type = other DNA
tgccatctgctgggtgctgtcgtttgccatcggcctgactcccatgcta

SEQ ID NO: 34 - DNA, 49 bp, synthetic construct
  misc_feature 1..49: note = T-2
  source 1..49: mol_type = other DNA
taggttggaacaactgcggtcagccaaaggagggcaagaaccactccca

SEQ ID NO: 35 - DNA, 49 bp, synthetic construct
  misc_feature 1..49: note = T-3
  source 1..49: mol_type = other DNA
tggagccctcggctcaaacctgggggcctggtaccctgcggctcccgaa

SEQ ID NO: 36 - DNA, 49 bp, synthetic construct
  misc_feature 1..49: note = T-4
  source 1..49: mol_type = other DNA
tgagcagagcaaccctgccccccaggtccagaaaccgcgtgccaaacca

SEQ ID NO: 37 - DNA, 49 bp, synthetic construct
  misc_feature 1..49: note = T-5
  source 1..49: mol_type = other DNA
tcctctgtgtccctgtggtccctggagcagccgttccgcatcgagctca

SEQ ID NO: 38 - DNA, 49 bp, synthetic construct
  misc_feature 1..49: note = T-6
  source 1..49: mol_type = other DNA
tttgcggaattggaatttcttagctgtgacacatccaggttacatggca

SEQ ID NO: 39 - DNA, 49 bp, synthetic construct
  misc_feature 1..49: note = T-7
  source 1..49: mol_type = other DNA
ttggaatttcttagctgtgacacatccaggttacatggcatttctcaca

SEQ ID NO: 40 - DNA, 49 bp, synthetic construct
  misc_feature 1..49: note = T-8
  source 1..49: mol_type = other DNA
tgtgacacatccaggttacatggcatttctcacatatgatgaagttaaa

SEQ ID NO: 41 - DNA, 49 bp, synthetic construct
  misc_feature 1..49: note = T-9
  source 1..49: mol_type = other DNA
ttataggcttcttctctggaatcttcttcatcatcctcctgacaatcga

SEQ ID NO: 42 - DNA, 49 bp, synthetic construct
  misc_feature 1..49: note = T-10
  source 1..49: mol_type = other DNA
tttgctgccatttctggaatgattctttcaatcatggacatacttaata

SEQ ID NO: 43 - DNA, 49 bp, synthetic construct
  misc_feature 1..49: note = T-11
  source 1..49: mol_type = other DNA
tggactgcctgccactgccccggcgcatggccgactacctccgacagta

SEQ ID NO: 44 - DNA, 49 bp, synthetic construct
  misc_feature 1..49: note = T-12
  source 1..49: mol_type = other DNA
tgcgcctagtgacccagcactgcctgctcctccaccagccactgctgta

SEQ ID NO: 45 - DNA, 49 bp, synthetic construct
  misc_feature 1..49: note = T-13
  source 1..49: mol_type = other DNA
tattacccaatggggacttggagaagcggagtgagccccagccagagga

SEQ ID NO: 46 - DNA, 49 bp, synthetic construct
  misc_feature 1..49: note = T-14
  source 1..49: mol_type = other DNA
tgaatgctgtggaagaaaaccaggggcccggggagtctcagaaggtgga

SEQ ID NO: 47 - DNA, 49 bp, synthetic construct
  misc_feature 1..49: note = T-15
  source 1..49: mol_type = other DNA
tgatggccaattctgccataagccctgtcctccaggtatgttacacaaa

SEQ ID NO: 48 - DNA, 49 bp, synthetic construct
  misc_feature 1..49: note = T-16
  source 1..49: mol_type = other DNA
ttaatgcccaagtgactgacatcaactccaagggattggaattgaggaa

SEQ ID NO: 49 - DNA, 49 bp, synthetic construct
  misc_feature 1..49: note = T-17
  source 1..49: mol_type = other DNA
tggggatgaaccagactgcgtgccctgccaagaagggaaggagtacaca

SEQ ID NO: 50 - DNA, 49 bp, synthetic construct
  misc_feature 1..49: note = T-18
  source 1..49: mol_type = other DNA
tgcagaagatgtagattgtgtgatgaaggacatggtaagagtcttaaaa

SEQ ID NO: 51 - DNA, 49 bp, synthetic construct
  misc_feature 1..49: note = T-19
  source 1..49: mol_type = other DNA
tttctagatgtgaacatggaatcatcaaggaatgcacactcaccagcaa

SEQ ID NO: 52 - DNA, 49 bp, synthetic construct
  misc_feature 1..49: note = T-20
  source 1..49: mol_type = other DNA
tcttgggggccccttccccacactatctcaatgcaaatatctgtctgaa

SEQ ID NO: 53 - DNA, 49 bp, synthetic construct
  misc_feature 1..49: note = T-21
  source 1..49: mol_type = other DNA
tgctactggccaacaccacctccgccttcccctacgcgctcctgagcaa

SEQ ID NO: 54 - DNA, 49 bp, synthetic construct
  misc_feature 1..49: note = T-22
  source 1..49: mol_type = other DNA
ttttaatagcattattcaaccaagaagttcaaattcccttgaccggtaa

SEQ ID NO: 55 - DNA, 49 bp, synthetic construct
  misc_feature 1..49: note = T-23
  source 1..49: mol_type = other DNA
ttccagaaagttactgtggcccatgtcctaaaaactggatatgttacaa

SEQ ID NO: 56 - DNA, 49 bp, synthetic construct
  misc_feature 1..49: note = T-24
  source 1..49: mol_type = other DNA
ttaggggacccattaggcatagaggactctctggaaagccaagattcaa

SEQ ID NO: 57 - DNA, 49 bp, synthetic construct
  misc_feature 1..49: note = T-25
  source 1..49: mol_type = other DNA
tctaagaagttcctgctctggagttgactaaagaatgtggttagagaca

SEQ ID NO: 58 - DNA, 49 bp, synthetic construct
  misc_feature 1..49: note = T-26
  source 1..49: mol_type = other DNA
tgcatatctgggctcagatgcttgtcattttccagtgataactccatca

SEQ ID NO: 59 - DNA, 49 bp, synthetic construct
  misc_feature 1..49: note = T-27
  source 1..49: mol_type = other DNA
tgcttgtcattttccagtgataactccatcaatgcctcctagtggtata

SEQ ID NO: 60 - DNA, 49 bp, synthetic construct
  misc_feature 1..49: note = T-28
  source 1..49: mol_type = other DNA
tagtgaaccttctctctctgggctccttcagatcaagaaattgaaacaa

SEQ ID NO: 61 - DNA, 49 bp, synthetic construct
  misc_feature 1..49: note = T-29
  source 1..49: mol_type = other DNA
tccaggtgaaagcagtcaaccaaatgtctccgatttgagtgataagaaa

SEQ ID NO: 62 - DNA, 49 bp, synthetic construct
  misc_feature 1..49: note = T-30
  source 1..49: mol_type = other DNA
tgacgcctggccggccggccgcgggactatccacctgcaagactatcga

SEQ ID NO: 63 - DNA, 49 bp, synthetic construct
  misc_feature 1..49: note = T-31
  source 1..49: mol_type = other DNA
tcgacatggagctggtgaagcggaagcgcatcgaggccatccgcggcca

SEQ ID NO: 64 - DNA, 49 bp, synthetic construct
  misc_feature 1..49: note = T-32
  source 1..49: mol_type = other DNA
tgaggccgactactacgccaaggaggtcacccgcgtgctaatggtggaa

SEQ ID NO: 65 - DNA, 49 bp, synthetic construct
  misc_feature 1..49: note = T-33
  source 1..49: mol_type = other DNA
tgatcgcctcccttcatttctccctgctagaaatctatgacaagttcaa

SEQ ID NO: 66 - DNA, 49 bp, synthetic construct
  misc_feature 1..49: note = T-34
  source 1..49: mol_type = other DNA
tggttaccattctctgtgtcaccccatgaaccataatggcctgctacca

SEQ ID NO: 67 - DNA, 49 bp, synthetic construct
  misc_feature 1..49: note = T-35
  source 1..49: mol_type = other DNA
tgccacatggccagctgactaccattaaccagtcacagctaagtgctca

SEQ ID NO: 68 - DNA, 49 bp, synthetic construct
  misc_feature 1..49: note = T-36
  source 1..49: mol_type = other DNA
tctgcctattcaccgattttgattctcaaacaaatgtgtcacaaagtaa

SEQ ID NO: 69 - DNA, 49 bp, synthetic construct
  misc_feature 1..49: note = T-37
  source 1..49: mol_type = other DNA
tgaggtctatggacttcaagagcaacagtgctgtggcctggagcaacaa

SEQ ID NO: 70 - DNA, 149 bp, synthetic construct
  misc_feature 1..149: note = TC-1
  source 1..149: mol_type = other DNA
gagatcatgtcctaaccctgatcctcttgtcccacagatatccagaaccctctaagaagttcctgctctaatataaatatatgaatgtggttagagacatgaccctgccgtgtaccagctgagagactctaaatccagtgacaagtctg

SEQ ID NO: 71 - DNA, 149 bp, synthetic construct
  misc_feature 1..149: note = TC-2
  source 1..149: mol_type = other DNA
gagatcatgtcctaaccctgatcctcttgtcccacagatatccagaaccctctaagaagttcctgcttcaatataaatatatgaatgtggttagagacatgaccctgccgtgtaccagctgagagactctaaatccagtgacaagtctg

SEQ ID NO: 72 - DNA, 149 bp, synthetic construct
  misc_feature 1..149: note = TC-3
  source 1..149: mol_type = other DNA
gagatcatgtcctaaccctgatcctcttgtcccacagatatccagaaccctctaagaagttcctgctatcatataaatatatgaatgtggttagagacatgaccctgccgtgtaccagctgagagactctaaatccagtgacaagtctg

SEQ ID NO: 73 - DNA, 149 bp, synthetic construct
  misc_feature 1..149: note = TC-4
  source 1..149: mol_type = other DNA
gagatcatgtcctaaccctgatcctcttgtcccacagatatccagaaccctctaagaagttcctgctaatctataaatatatgaatgtggttagagacatgaccctgccgtgtaccagctgagagactctaaatccagtgacaagtctg

SEQ ID NO: 74 - DNA, 149 bp, synthetic construct
  misc_feature 1..149: note = TC-5
  source 1..149: mol_type = other DNA
gagatcatgtcctaaccctgatcctcttgtcccacagatatccagaaccctctaagaagttcctgctatatcataaatatatgaatgtggttagagacatgaccctgccgtgtaccagctgagagactctaaatccagtgacaagtctg

SEQ ID NO: 75 - DNA, 149 bp, synthetic construct
  misc_feature 1..149: note = TC-6
  source 1..149: mol_type = other DNA
gagatcatgtcctaaccctgatcctcttgtcccacagatatccagaaccctctaagaagttcctgctataatctaaatatatgaatgtggttagagacatgaccctgccgtgtaccagctgagagactctaaatccagtgacaagtctg

SEQ ID NO: 76 - DNA, 149 bp, synthetic construct
  misc_feature 1..149: note = TC-7
  source 1..149: mol_type = other DNA
gagatcatgtcctaaccctgatcctcttgtcccacagatatccagaaccctctaagaagttcctgctaataatcaaatatatgaatgtggttagagacatgaccctgccgtgtaccagctgagagactctaaatccagtgacaagtctg

SEQ ID NO: 77 - skipped

SEQ ID NO: 78 - DNA, 149 bp, synthetic construct
  misc_feature 1..149: note = TC-8
  source 1..149: mol_type = other DNA
gagatcatgtcctaaccctgatcctcttgtcccacagatatccagaaccctctaagaagttcctgcttataaatcaatatatgaatgtggttagagacatgaccctgccgtgtaccagctgagagactctaaatccagtgacaagtctg

SEQ ID NO: 79 - DNA, 149 bp, synthetic construct
  misc_feature 1..149: note = TC-9
  source 1..149: mol_type = other DNA
gagatcatgtcctaaccctgatcctcttgtcccacagatatccagaaccctctaagaagttcctgcttataatatcatatatgaatgtggttagagacatgaccctgccgtgtaccagctgagagactctaaatccagtgacaagtctg

SEQ ID NO: 80 - DNA, 149 bp, synthetic construct
  misc_feature 1..149: note = TC-10
  source 1..149: mol_type = other DNA
gagatcatgtcctaaccctgatcctcttgtcccacagatatccagaaccctctaagaagttcctgcttaatataatctatatgaatgtggttagagacatgaccctgccgtgtaccagctgagagactctaaatccagtgacaagtctg

SEQ ID NO: 81 - DNA, 149 bp, synthetic construct
  misc_feature 1..149: note = TC-11
  source 1..149: mol_type = other DNA
gagatcatgtcctaaccctgatcctcttgtcccacagatatccagaaccctctaagaagttcctgcttaatataaatcatatgaatgtggttagagacatgaccctgccgtgtaccagctgagagactctaaatccagtgacaagtctg

SEQ ID NO: 82 - DNA, 149 bp, synthetic construct
  misc_feature 1..149: note = TC-12
  source 1..149: mol_type = other DNA
gagatcatgtcctaaccctgatcctcttgtcccacagatatccagaaccctctaagaagttcctgctaataataatatctatgaatgtggttagagacatgaccctgccgtgtaccagctgagagactctaaatccagtgacaagtctg

SEQ ID NO: 83 - DNA, 149 bp, synthetic construct
  misc_feature 1..149: note = TC-13
  source 1..149: mol_type = other DNA
gagatcatgtcctaaccctgatcctcttgtcccacagatatccagaaccctctaagaagttcctgctaatataaataatcatgaatgtggttagagacatgaccctgccgtgtaccagctgagagactctaaatccagtgacaagtctg

SEQ ID NO: 84 - DNA, 149 bp, synthetic construct
  misc_feature 1..149: note = TC-14
  source 1..149: mol_type = other DNA
gagatcatgtcctaaccctgatcctcttgtcccacagatatccagaaccctctaagaagttcctgctaatataaatatatctgaatgtggttagagacatgaccctgccgtgtaccagctgagagactctaaatccagtgacaagtctg

SEQ ID NO: 85 - DNA, 149 bp, synthetic construct
  misc_feature 1..149: note = TC-15
  source 1..149: mol_type = other DNA
gagatcatgtcctaaccctgatcctcttgtcccacagatatccagaaccctctaagaagttcctgctaatataaatataatcgaatgtggttagagacatgaccctgccgtgtaccagctgagagactctaaatccagtgacaagtctg

SEQ ID NO: 86 - DNA, 149 bp, synthetic construct
  misc_feature 1..149: note = GA-1
  source 1..149: mol_type = other DNA
gagatcatgtcctaaccctgatcctcttgtcccacagatatccagaaccctctaagaagttcctgctatatatttatattaggaatgtggttagagacatgaccctgccgtgtaccagctgagagactctaaatccagtgacaagtctg

SEQ ID NO: 87 - DNA, 149 bp, synthetic construct
  misc_feature 1..149: note = GA-2
  source 1..149: mol_type = other DNA
gagatcatgtcctaaccctgatcctcttgtcccacagatatccagaaccctctaagaagttcctgctatatatttatattgagaatgtggttagagacatgaccctgccgtgtaccagctgagagactctaaatccagtgacaagtctg

SEQ ID NO: 88 - DNA, 149 bp, synthetic construct
  misc_feature 1..149: note = GA-3
  source 1..149: mol_type = other DNA
gagatcatgtcctaaccctgatcctcttgtcccacagatatccagaaccctctaagaagttcctgctatatatttatatgatgaatgtggttagagacatgaccctgccgtgtaccagctgagagactctaaatccagtgacaagtctg

SEQ ID NO: 89 - DNA, 149 bp, synthetic construct
  misc_feature 1..149: note = GA-4
  source 1..149: mol_type = other DNA
gagatcatgtcctaaccctgatcctcttgtcccacagatatccagaaccctctaagaagttcctgctatatatttatagattgaatgtggttagagacatgaccctgccgtgtaccagctgagagactctaaatccagtgacaagtctg

SEQ ID NO: 90 - DNA, 149 bp, synthetic construct
  misc_feature 1..149: note = GA-5
  source 1..149: mol_type = other DNA
gagatcatgtcctaaccctgatcctcttgtcccacagatatccagaaccctctaagaagttcctgctatatatttatgatatgaatgtggttagagacatgaccctgccgtgtaccagctgagagactctaaatccagtgacaagtctg

SEQ ID NO: 91 - DNA, 149 bp, synthetic construct
  misc_feature 1..149: note = GA-6
  source 1..149: mol_type = other DNA
gagatcatgtcctaaccctgatcctcttgtcccacagatatccagaaccctctaagaagttcctgctatatatttagattatgaatgtggttagagacatgaccctgccgtgtaccagctgagagactctaaatccagtgacaagtctg

SEQ ID NO: 92 - DNA, 149 bp, synthetic construct
  misc_feature 1..149: note = GA-7
  source 1..149: mol_type = other DNA
gagatcatgtcctaaccctgatcctcttgtcccacagatatccagaaccctctaagaagttcctgctatatatttgattattgaatgtggttagagacatgaccctgccgtgtaccagctgagagactctaaatccagtgacaagtctg

SEQ ID NO: 93 - DNA, 149 bp, synthetic construct
  misc_feature 1..149: note = GA-8
  source 1..149: mol_type = other DNA
gagatcatgtcctaaccctgatcctcttgtcccacagatatccagaaccctctaagaagttcctgctatatattgatttatagaatgtggttagagacatgaccctgccgtgtaccagctgagagactctaaatccagtgacaagtctg

SEQ ID NO: 94 - DNA, 149 bp, synthetic construct
  misc_feature 1..149: note = GA-9
  source 1..149: mol_type = other DNA
gagatcatgtcctaaccctgatcctcttgtcccacagatatccagaaccctctaagaagttcctgctatatatgatattatagaatgtggttagagacatgaccctgccgtgtaccagctgagagactctaaatccagtgacaagtctg

SEQ ID NO: 95 - DNA, 149 bp, synthetic construct
  misc_feature 1..149: note = GA-10
  source 1..149: mol_type = other DNA
gagatcatgtcctaaccctgatcctcttgtcccacagatatccagaaccctctaagaagttcctgctatatagattatattagaatgtggttagagacatgaccctgccgtgtaccagctgagagactctaaatccagtgacaagtctg

SEQ ID NO: 96 - DNA, 149 bp, synthetic construct
  misc_feature 1..149: note = GA-11
  source 1..149: mol_type = other DNA
gagatcatgtcctaaccctgatcctcttgtcccacagatatccagaaccctctaagaagttcctgctatatgatttatattagaatgtggttagagacatgaccctgccgtgtaccagctgagagactctaaatccagtgacaagtctg

SEQ ID NO: 97 - DNA, 149 bp, synthetic construct
  misc_feature 1..149: note = GA-12
  source 1..149: mol_type = other DNA
gagatcatgtcctaaccctgatcctcttgtcccacagatatccagaaccctctaagaagttcctgctatagatattattattgaatgtggttagagacatgaccctgccgtgtaccagctgagagactctaaatccagtgacaagtctg

SEQ ID NO: 98 - DNA, 149 bp, synthetic construct
  misc_feature 1..149: note = GA-13
  source 1..149: mol_type = other DNA
gagatcatgtcctaaccctgatcctcttgtcccacagatatccagaaccctctaagaagttcctgctatgattatttatattgaatgtggttagagacatgaccctgccgtgtaccagctgagagactctaaatccagtgacaagtctg

SEQ ID NO: 99 - DNA, 149 bp, synthetic construct
  misc_feature 1..149: note = GA-14
  source 1..149: mol_type = other DNA
gagatcatgtcctaaccctgatcctcttgtcccacagatatccagaaccctctaagaagttcctgctagatatatttatattgaatgtggttagagacatgaccctgccgtgtaccagctgagagactctaaatccagtgacaagtctg

SEQ ID NO: 100 - DNA, 149 bp, synthetic construct
  misc_feature 1..149: note = GA-15
  source 1..149: mol_type = other DNA
gagatcatgtcctaaccctgatcctcttgtcccacagatatccagaaccctctaagaagttcctgctgattatatttatattgaatgtggttagagacatgaccctgccgtgtaccagctgagagactctaaatccagtgacaagtctg

SEQ ID NO: 101 - DNA, 59 bp, Homo sapiens
  misc_feature 1..59: note = TRAC TALEN target sequence
  source 1..59: mol_type = genomic DNA
tgatcctcttgtcccacagatatccagaaccctgaccctgccgtgtaccagctgagaga

SEQ ID NO: 102 - DNA, 58 bp, Homo sapiens; identical to SEQ ID NO: 32
  misc_feature 1..58: note = CD52 TALEN Target sequence
  source 1..58: mol_type = genomic DNA

SEQ ID NO: 103 - DNA, 145 bp, synthetic construct
  misc_feature 1..145: note = TCGA-1
  source 1..145: mol_type = other DNA
gagatcatgtcctaaccctgatcctcttgtcccacagatatccagaaccctctaagaagttcctgcttcgatgaatgtggttagagacaaaacagtgaccctgccgtgtaccagctgagagactctaaatccagtgacaagtctg

SEQ ID NO: 104 - DNA, 145 bp, synthetic construct
  misc_feature 1..145: note = TCGA-2
  source 1..145: mol_type = other DNA
gagatcatgtcctaaccctgatcctcttgtcccacagatatccagaaccctctaagaagttcctgctatcgagaatgtggttagagacaaaaccttgaccctgccgtgtaccagctgagagactctaaatccagtgacaagtctg

SEQ ID NO: 105 - DNA, 145 bp, synthetic construct
  misc_feature 1..145: note = TCGA-3
  source 1..145: mol_type = other DNA
gagatcatgtcctaaccctgatcctcttgtcccacagatatccagaaccctctaagaagttcctgctgatcagaatgtggttagagacaaaacgatgaccctgccgtgtaccagctgagagactctaaatccagtgacaagtctg

SEQ ID NO: 106 - DNA, 145 bp, synthetic construct
  misc_feature 1..145: note = TCGA-4
  source 1..145: mol_type = other DNA
gagatcatgtcctaaccctgatcctcttgtcccacagatatccagaaccctctaagaagttcctgctagatcgaatgtggttagagacaaaacggtgaccctgccgtgtaccagctgagagactctaaatccagtgacaagtctg

SEQ ID NO: 107 - DNA, 147 bp, synthetic construct
  misc_feature 1..147: note = TCGA-5
  source 1..147: mol_type = other DNA
gagatcatgtcctaaccctgatcctcttgtcccacagatatccagaaccctctaagaagttcctgcttcgatatgaatgtggttagagacaaaactctgaccctgccgtgtaccagctgagagactctaaatccagtgacaagtctg

SEQ ID NO: 108 - DNA, 147 bp, synthetic construct
  misc_feature 1..147: note = TCGA-6
  source 1..147: mol_type = other DNA
gagatcatgtcctaaccctgatcctcttgtcccacagatatccagaaccctctaagaagttcctgctaatcgatgaatgtggttagagacaaaagactgaccctgccgtgtaccagctgagagactctaaatccagtgacaagtctg

SEQ ID NO: 109 - DNA, 147 bp, synthetic construct
  misc_feature 1..147: note = TCGA-7
  source 1..147: mol_type = other DNA
gagatcatgtcctaaccctgatcctcttgtcccacagatatccagaaccctctaagaagttcctgctgatatcagaatgtggttagagacaaaagagtgaccctgccgtgtaccagctgagagactctaaatccagtgacaagtctg

SEQ ID NO: 110 - DNA, 149 bp, synthetic construct
  misc_feature 1..149: note = TCGA-8
  source 1..149: mol_type = other DNA
gagatcatgtcctaaccctgatcctcttgtcccacagatatccagaaccctctaagaagttcctgcttcgatattagaatgtggttagagacaaaagcttgaccctgccgtgtaccagctgagagactctaaatccagtgacaagtctg

SEQ ID NO: 111 - DNA, 149 bp, synthetic construct
  misc_feature 1..149: note = TCGA-9
  source 1..149: mol_type = other DNA
gagatcatgtcctaaccctgatcctcttgtcccacagatatccagaaccctctaagaagttcctgcttatcgatatgaatgtggttagagacaaaaggatgaccctgccgtgtaccagctgagagactctaaatccagtgacaagtctg

SEQ ID NO: 112 - DNA, 149 bp, synthetic construct
  misc_feature 1..149: note = TCGA-10
  source 1..149: mol_type = other DNA
gagatcatgtcctaaccctgatcctcttgtcccacagatatccagaaccctctaagaagttcctgctattatcgatgaatgtggttagagacaaaagggtgaccctgccgtgtaccagctgagagactctaaatccagtgacaagtctg

SEQ ID NO: 113 - DNA, 149 bp, synthetic construct
  misc_feature 1..149: note = TCGA-11
  source 1..149: mol_type = other DNA
gagatcatgtcctaaccctgatcctcttgtcccacagatatccagaaccctctaagaagttcctgctatattatcggaatgtggttagagacaaaagtctgaccctgccgtgtaccagctgagagactctaaatccagtgacaagtctg

SEQ ID NO: 114 - DNA, 151 bp, synthetic construct
  misc_feature 1..151: note = TCGA-12
  source 1..151: mol_type = other DNA
gagatcatgtcctaaccctgatcctcttgtcccacagatatccagaaccctctaagaagttcctgcttcgatatttaagaatgtggttagagacaaaatcctgaccctgccgtgtaccagctgagagactctaaatccagtgacaagtctg

SEQ ID NO: 115 - DNA, 151 bp, synthetic construct
  misc_feature 1..151: note = TCGA-13
  source 1..151: mol_type = other DNA
gagatcatgtcctaaccctgatcctcttgtcccacagatatccagaaccctctaagaagttcctgctaatcgatatttgaatgtggttagagacaaaatgctgaccctgccgtgtaccagctgagagactctaaatccagtgacaagtctg

SEQ ID NO: 116 - DNA, 151 bp, synthetic construct
  misc_feature 1..151: note = TCGA-14
  source 1..151: mol_type = other DNA
gagatcatgtcctaaccctgatcctcttgtcccacagatatccagaaccctctaagaagttcctgctttaatcgatatgaatgtggttagagacaaacaagtgaccctgccgtgtaccagctgagagactctaaatccagtgacaagtctg

SEQ ID NO: 117 - DNA, 151 bp, synthetic construct
  misc_feature 1..151: note = TCGA-15
  source 1..151: mol_type = other DNA
gagatcatgtcctaaccctgatcctcttgtcccacagatatccagaaccctctaagaagttcctgctatttaatcgatgaatgtggttagagacaaacacctgaccctgccgtgtaccagctgagagactctaaatccagtgacaagtctg

SEQ ID NO: 118 - DNA, 151 bp, synthetic construct
  misc_feature 1..151: note = TCGA-16
  source 1..151: mol_type = other DNA
gagatcatgtcctaaccctgatcctcttgtcccacagatatccagaaccctctaagaagttcctgctatatttaatcggaatgtggttagagacaaacagatgaccctgccgtgtaccagctgagagactctaaatccagtgacaagtctg

SEQ ID NO: 119 - DNA, 153 bp, synthetic construct
  misc_feature 1..153: note = TCGA-17
  source 1..153: mol_type = other DNA
gagatcatgtcctaaccctgatcctcttgtcccacagatatccagaaccctctaagaagttcctgcttcgataattataagaatgtggttagagacaaacaggtgaccctgccgtgtaccagctgagagactctaaatccagtgacaagtctg

SEQ ID NO: 120 - DNA, 153 bp, synthetic construct
  misc_feature 1..153: note = TCGA-18
  source 1..153: mol_type = other DNA
gagatcatgtcctaaccctgatcctcttgtcccacagatatccagaaccctctaagaagttcctgctaatcgataattatgaatgtggttagagacaaaccattgaccctgccgtgtaccagctgagagactctaaatccagtgacaagtctg

SEQ ID NO: 121 - DNA, 153 bp, synthetic construct
  misc_feature 1..153: note = TCGA-19
  source 1..153: mol_type = other DNA
gagatcatgtcctaaccctgatcctcttgtcccacagatatccagaaccctctaagaagttcctgctataatcgataattgaatgtggttagagacaaaccgttgaccctgccgtgtaccagctgagagactctaaatccagtgacaagtctg

SEQ ID NO: 122 - DNA, 153 bp, synthetic construct
  misc_feature 1..153: note = TCGA-20
  source 1..153: mol_type = other DNA
gagatcatgtcctaaccctgatcctcttgtcccacagatatccagaaccctctaagaagttcctgctttataatcgataagaatgtggttagagacaaacgcatgaccctgccgtgtaccagctgagagactctaaatccagtgacaagtctg

SEQ ID NO: 123 - DNA, 153 bp, synthetic construct
  misc_feature 1..153: note = TCGA-21
  source 1..153: mol_type = other DNA
gagatcatgtcctaaccctgatcctcttgtcccacagatatccagaaccctctaagaagttcctgctaattataatcgatgaatgtggttagagacaaactcgtgaccctgccgtgtaccagctgagagactctaaatccagtgacaagtctg

SEQ ID NO: 124 - DNA, 153 bp, synthetic construct
  misc_feature 1..153: note = TCGA-22
  source 1..153: mol_type = other DNA
gagatcatgtcctaaccctgatcctcttgtcccacagatatccagaaccctctaagaagttcctgctataattataatcggaatgtggttagagacaaactggtgaccctgccgtgtaccagctgagagactctaaatccagtgacaagtctg

SEQ ID NO: 125 - DNA, 155 bp, synthetic construct
  misc_feature 1..155: note = TCGA-23
  source 1..155: mol_type = other DNA
gagatcatgtcctaaccctgatcctcttgtcccacagatatccagaaccctctaagaagttcctgcttcgattataattaaagaatgtggttagagacaaacttctgaccctgccgtgtaccagctgagagactctaaatccagtgacaagtctg

SEQ ID NO: 126 - DNA, 155 bp, synthetic construct
  misc_feature 1..155: note = TCGA-24
  source 1..155: mol_type = other DNA
gagatcatgtcctaaccctgatcctcttgtcccacagatatccagaaccctctaagaagttcctgctaatcgattataattagaatgtggttagagacaaagaactgaccctgccgtgtaccagctgagagactctaaatccagtgacaagtctg

SEQ ID NO: 127 - DNA, 155 bp, synthetic construct
  misc_feature 1..155: note = TCGA-25
  source 1..155: mol_type = other DNA
gagatcatgtcctaaccctgatcctcttgtcccacagatatccagaaccctctaagaagttcctgcttaaatcgattataatgaatgtggttagagacaaagaagtgaccctgccgtgtaccagctgagagactctaaatccagtgacaagtctg

SEQ ID NO: 128 - DNA, 155 bp, synthetic construct
  misc_feature 1..155: note = TCGA-26
  source 1..155: mol_type = other DNA
gagatcatgtcctaaccctgatcctcttgtcccacagatatccagaaccctctaagaagttcctgctattaaatcgattatagaatgtggttagagacaaagacatgaccctgccgtgtaccagctgagagactctaaatccagtgacaagtctg

SEQ ID NO: 129 - DNA, 155 bp, synthetic construct
  misc_feature 1..155: note = TCGA-27
  source 1..155: mol_type = other DNA
gagatcatgtcctaaccctgatcctcttgtcccacagatatccagaaccctctaagaagttcctgcttaattaaatcgattagaatgtggttagagacaaagacttgaccctgccgtgtaccagctgagagactctaaatccagtgacaagtctg

SEQ ID NO: 130 - DNA, 155 bp, synthetic construct
  misc_feature 1..155: note = TCGA-28
  source 1..155: mol_type = other DNA
gagatcatgtcctaaccctgatcctcttgtcccacagatatccagaaccctctaagaagttcctgcttataattaaatcgatgaatgtggttagagacaaagagatgaccctgccgtgtaccagctgagagactctaaatccagtgacaagtctg

SEQ ID NO: 131 - DNA, 155 bp, synthetic construct
  misc_feature 1..155: note = TCGA-29
  source 1..155: mol_type = other DNA
gagatcatgtcctaaccctgatcctcttgtcccacagatatccagaaccctctaagaagttcctgctattataattaaatcggaatgtggttagagacaaagagttgaccctgccgtgtaccagctgagagactctaaatccagtgacaagtctg

SEQ ID NO: 132 - DNA, 157 bp, synthetic construct
  misc_feature 1..157: note = TCGA-30
  source 1..157: mol_type = other DNA
gagatcatgtcctaaccctgatcctcttgtcccacagatatccagaaccctctaagaagttcctgcttcgatattattaaattagaatgtggttagagacaaagatctgaccctgccgtgtaccagctgagagactctaaatccagtgacaagtctg

SEQ ID NO: 133 - DNA, 157 bp, synthetic construct
  misc_feature 1..157: note = TCGA-31
  source 1..157: mol_type = other DNA
gagatcatgtcctaaccctgatcctcttgtcccacagatatccagaaccctctaagaagttcctgcttatcgatattattaaatgaatgtggttagagacaaagctgtgaccctgccgtgtaccagctgagagactctaaatccagtgacaagtctg

SEQ ID NO: 134 - DNA, 157 bp, synthetic construct
  misc_feature 1..157: note = TCGA-32
  source 1..157: mol_type = other DNA
gagatcatgtcctaaccctgatcctcttgtcccacagatatccagaaccctctaagaagttcctgctattatcgatattattaagaatgtggttagagacaaaggaatgaccctgccgtgtaccagctgagagactctaaatccagtgacaagtctg

SEQ ID NO: 135 - DNA, 157 bp, synthetic construct
  misc_feature 1..157: note = TCGA-33
  source 1..157: mol_type = other DNA
gagatcatgtcctaaccctgatcctcttgtcccacagatatccagaaccctctaagaagttcctgctaaattatcgatattattgaatgtggttagagacaaaggagtgaccctgccgtgtaccagctgagagactctaaatccagtgacaagtctg

SEQ ID NO: 136 - DNA, 157 bp, synthetic construct
  misc_feature 1..157: note = TCGA-34
  source 1..157: mol_type = other DNA
gagatcatgtcctaaccctgatcctcttgtcccacagatatccagaaccctctaagaagttcctgctttaaattatcgatattagaatgtggttagagacaaagggatgaccctgccgtgtaccagctgagagactctaaatccagtgacaagtctg

SEQ ID NO: 137 - DNA, 157 bp, synthetic construct
  misc_feature 1..157: note = TCGA-35
  source 1..157: mol_type = other DNA
gagatcatgtcctaaccctgatcctcttgtcccacagatatccagaaccctctaagaagttcctgcttattaaattatcgatatgaatgtggttagagacaaagggttgaccctgccgtgtaccagctgagagactctaaatccagtgacaagtctg

SEQ ID NO: 138 - DNA, 157 bp, synthetic construct
  misc_feature 1..157: note = TCGA-36
  source 1..157: mol_type = other DNA
gagatcatgtcctaaccctgatcctcttgtcccacagatatccagaaccctctaagaagttcctgctattattaaattatcgatgaatgtggttagagacaaaggtctgaccctgccgtgtaccagctgagagactctaaatccagtgacaagtctg

SEQ ID NO: 139 - DNA, 157 bp, synthetic construct
  misc_feature 1..157: note = TCGA-37
  source 1..157: mol_type = other DNA
gagatcatgtcctaaccctgatcctcttgtcccacagatatccagaaccctctaagaagttcctgctatattattaaattatcggaatgtggttagagacaaaggtgtgaccctgccgtgtaccagctgagagactctaaatccagtgacaagtctg

SEQ ID NO: 140 - DNA, 159 bp, synthetic construct
  misc_feature 1..159: note = TCGA-38
  source 1..159: mol_type = other DNA
gagatcatgtcctaaccctgatcctcttgtcccacagatatccagaaccctctaagaagttcctgcttcgatataattatattatagaatgtggttagagacaaagtactgaccctgccgtgtaccagctgagagactctaaatccagtgacaagtctg

SEQ ID NO: 141 - DNA, 159 bp, synthetic construct
  misc_feature 1..159: note = TCGA-39
  source 1..159: mol_type = other DNA
gagatcatgtcctaaccctgatcctcttgtcccacagatatccagaaccctctaagaagttcctgcttatcgatataattatattagaatgtggttagagacaaagtcgtgaccctgccgtgtaccagctgagagactctaaatccagtgacaagtctg

SEQ ID NO: 142 - DNA, 159 bp, synthetic construct
  misc_feature 1..159: note = TCGA-40
  source 1..159: mol_type = other DNA
gagatcatgtcctaaccctgatcctcttgtcccacagatatccagaaccctctaagaagttcctgcttatatcgatataattatatgaatgtggttagagacaaatacctgaccctgccgtgtaccagctgagagactctaaatccagtgacaagtctg

SEQ ID NO: 143 - DNA, 159 bp, synthetic construct
  misc_feature 1..159: note = TCGA-41
  source 1..159: mol_type = other DNA
gagatcatgtcctaaccctgatcctcttgtcccacagatatccagaaccctctaagaagttcctgctattatatcgatataattatgaatgtggttagagacaaatcgatgaccctgccgtgtaccagctgagagactctaaatccagtgacaagtctg

SEQ ID NO: 144 - DNA, 159 bp, synthetic construct
  misc_feature 1..159: note = TCGA-42
  source 1..159: mol_type = other DNA
gagatcatgtcctaaccctgatcctcttgtcccacagatatccagaaccctctaagaagttcctgctatattatatcgatataattgaatgtggttagagacaaatctctgaccctgccgtgtaccagctgagagactctaaatccagtgacaagtctg

SEQ ID NO: 145 - DNA, 159 bp, synthetic construct
  misc_feature 1..159: note = TCGA-43
  source 1..159: mol_type = other DNA
gagatcatgtcctaaccctgatcctcttgtcccacagatatccagaaccctctaagaagttcctgctttatattatatcgatataagaatgtggttagagacaaattcctgaccctgccgtgtaccagctgagagactctaaatccagtgacaagtctg

SEQ ID NO: 146 - DNA, 159 bp, synthetic construct
  misc_feature 1..159: note = TCGA-44
  source 1..159: mol_type = other DNA
gagatcatgtcctaaccctgatcctcttgtcccacagatatccagaaccctctaagaagttcctgctaattatattatatcgatatgaatgtggttagagacaaattgctgaccctgccgtgtaccagctgagagactctaaatccagtgacaagtctg

SEQ ID NO: 147 - DNA, 159 bp, synthetic construct
  misc_feature 1..159: note = TCGA-45
  source 1..159: mol_type = other DNA
gagatcatgtcctaaccctgatcctcttgtcccacagatatccagaaccctctaagaagttcctgctataattatattatatcgatgaatgtggttagagacaaattggtgaccctgccgtgtaccagctgagagactctaaatccagtgacaagtctg

SEQ ID NO: 148 - DNA, 159 bp, synthetic construct
  misc_feature 1..159: note = TCGA-46
  source 1..159: mol_type = other DNA
gagatcatgtcctaaccctgatcctcttgtcccacagatatccagaaccctctaagaagttcctgctatataattatattatatcggaatgtggttagagacaacaacgtgaccctgccgtgtaccagctgagagactctaaatccagtgacaagtctg

SEQ ID NO: 149 - DNA, 161 bp, synthetic construct
  misc_feature 1..161: note = TCGA-47
  source 1..161: mol_type = other DNA
gagatcatgtcctaaccctgatcctcttgtcccacagatatccagaaccctctaagaagttcctgcttcgatataaatatattatttagaatgtggttagagacaacaagctgaccctgccgtgtaccagctgagagactctaaatccagtgacaagtctg

SEQ ID NO: 150 - DNA, 161 bp, synthetic construct
  misc_feature 1..161: note = TCGA-48
  source 1..161: mol_type = other DNA
gagatcatgtcctaaccctgatcctcttgtcccacagatatccagaaccctctaagaagttcctgcttatcgatataaatatattattgaatgtggttagagacaacaagttgaccctgccgtgtaccagctgagagactctaaatccagtgacaagtctg

SEQ ID NO: 151 - DNA, 161 bp, synthetic construct
  misc_feature 1..161: note = TCGA-49
  source 1..161: mol_type = other DNA
gagatcatgtcctaaccctgatcctcttgtcccacagatatccagaaccctctaagaagttcctgcttttatcgatataaatatattagaatgtggttagagacaacaccatgaccctgccgtgtaccagctgagagactctaaatccagtgacaagtctg

SEQ ID NO: 152 - DNA, 161 bp, synthetic construct
  misc_feature 1..161: note = TCGA-50
  source 1..161: mol_type = other DNA
gagatcatgtcctaaccctgatcctcttgtcccacagatatccagaaccctctaagaagttcctgcttatttatcgatataaatatatgaatgtggttagagacaacactgtgaccctgccgtgtaccagctgagagactctaaatccagtgacaagtctg

SEQ ID NO: 153 - DNA, 161 bp, synthetic construct
  misc_feature 1..161: note = TCGA-51
  source 1..161: mol_type = other DNA
gagatcatgtcctaaccctgatcctcttgtcccacagatatccagaaccctctaagaagttcctgctattatttatcgatataaatatgaatgtggttagagacaacagtgtgaccctgccgtgtaccagctgagagactctaaatccagtgacaagtctg

SEQ ID NO: 154 - DNA, 161 bp, synthetic construct
  misc_feature 1..161: note = TCGA-52
  source 1..161: mol_type = other DNA
gagatcatgtcctaaccctgatcctcttgtcccacagatatccagaaccctctaagaagttcctgctatattatttatcgatataaatgaatgtggttagagacaacatagtgaccctgccgtgtaccagctgagagactctaaatccagtgacaagtctg

SEQ ID NO: 155 - DNA, 161 bp, synthetic construct
  misc_feature 1..161: note = TCGA-53
  source 1..161: mol_type = other DNA
gagatcatgtcctaaccctgatcctcttgtcccacagatatccagaaccctctaagaagttcctgctatatattatttatcgatataagaatgtggttagagacaacatcttgaccctgccgtgtaccagctgagagactctaaatccagtgacaagtctg

SEQ ID NO: 156 - DNA, 161 bp, synthetic construct
  misc_feature 1..161: note = TCGA-54
  source 1..161: mol_type = other DNA
gagatcatgtcctaaccctgatcctcttgtcccacagatatccagaaccctctaagaagttcctgctaaatatattatttatcgatatgaatgtggttagagacaaccgaatgaccctgccgtgtaccagctgagagactctaaatccagtgacaagtctg

SEQ ID NO: 157 - DNA, 161 bp, synthetic construct
  misc_feature 1..161: note = TCGA-55
  source 1..161: mol_type = other DNA
gagatcatgtcctaaccctgatcctcttgtcccacagatatccagaaccctctaagaagttcctgctataaatatattatttatcgatgaatgtggttagagacaacctactgaccctgccgtgtaccagctgagagactctaaatccagtgacaagtctg

SEQ ID NO: 158 - DNA, 161 bp, synthetic construct
  misc_feature 1..161: note = TCGA-56
  source 1..161: mol_type = other DNA
gagatcatgtcctaaccctgatcctcttgtcccacagatatccagaaccctctaagaagttcctgctatataaatatattatttatcggaatgtggttagagacaacctgatgaccctgccgtgtaccagctgagagactctaaatccagtgacaagtctg

SEQ ID NO: 159 - DNA, 163 bp, synthetic construct
  misc_feature 1..163: note = TCGA-57
  source 1..163: mol_type = other DNA
gagatcatgtcctaaccctgatcctcttgtcccacagatatccagaaccctctaagaagttcctgcttcgatattaaatatattaatttagaatgtggttagagacaacctgttgaccctgccgtgtaccagctgagagactctaaatccagtgacaagtctg

SEQ ID NO: 160 - DNA, 163 bp, synthetic construct
  misc_feature 1..163: note = TCGA-58
  source 1..163: mol_type = other DNA
gagatcatgtcctaaccctgatcctcttgtcccacagatatccagaaccctctaagaagttcctgcttatcgatattaaatatattaattgaatgtggttagagacaacgaattgaccctgccgtgtaccagctgagagactctaaatccagtgacaagtctg

SEQ ID NO: 161 - DNA, 163 bp, synthetic construct
  misc_feature 1..163: note = TCGA-59
  source 1..163: mol_type = other DNA
gagatcatgtcctaaccctgatcctcttgtcccacagatatccagaaccctctaagaagttcctgcttttatcgatattaaatatattaagaatgtggttagagacaacggtttgaccctgccgtgtaccagctgagagactctaaatccagtgacaagtctg

SEQ ID NO: 162 - DNA, 163 bp, synthetic construct
  misc_feature 1..163: note = TCGA-60
  source 1..163: mol_type = other DNA
gagatcatgtcctaaccctgatcctcttgtcccacagatatccagaaccctctaagaagttcctgctaatttatcgatattaaatatattgaatgtggttagagacaacgtagtgaccctgccgtgtaccagctgagagactctaaatccagtgacaagtctg

SEQ ID NO: 163 - DNA, 163 bp, synthetic construct
  misc_feature 1..163: note = TCGA-61
  source 1..163: mol_type = other DNA
gagatcatgtcctaaccctgatcctcttgtcccacagatatccagaaccctctaagaagttcctgctttaatttatcgatattaaatatagaatgtggttagagacaacgtcttgaccctgccgtgtaccagctgagagactctaaatccagtgacaagtctg

SEQ ID NO: 164 - DNA, 163 bp, synthetic construct
  misc_feature 1..163: note = TCGA-62
  source 1..163: mol_type = other DNA
gagatcatgtcctaaccctgatcctcttgtcccacagatatccagaaccctctaagaagttcctgcttattaatttatcgatattaaatagaatgtggttagagacaacgttatgaccctgccgtgtaccagctgagagactctaaatccagtgacaagtctg

SEQ ID NO: 165 - DNA, 163 bp, synthetic construct
  misc_feature 1..163: note = TCGA-63
  source 1..163: mol_type = other DNA
gagatcatgtcctaaccctgatcctcttgtcccacagatatccagaaccctctaagaagttcctgcttatattaatttatcgatattaaagaatgtggttagagacaactacttgaccctgccgtgtaccagctgagagactctaaatccagtgacaagtctg

SEQ ID NO: 166 - DNA, 163 bp, synthetic construct
  misc_feature 1..163: note = TCGA-64
  source 1..163: mol_type = other DNA
gagatcatgtcctaaccctgatcctcttgtcccacagatatccagaaccctctaagaagttcctgctaatatattaatttatcgatattagaatgtggttagagacaactcaatgaccctgccgtgtaccagctgagagactctaaatccagtgacaagtctg

SEQ ID NO: 167 - DNA, 163 bp, synthetic construct
  misc_feature 1..163: note = TCGA-65
  source 1..163: mol_type = other DNA
gagatcatgtcctaaccctgatcctcttgtcccacagatatccagaaccctctaagaagttcctgcttaaatatattaatttatcgatatgaatgtggttagagacaactctgtgaccctgccgtgtaccagctgagagactctaaatccagtgacaagtctg

SEQ ID NO: 168 - DNA, 163 bp, synthetic construct
  misc_feature 1..163: note = TCGA-66
  source 1..163: mol_type = other DNA
gagatcatgtcctaaccctgatcctcttgtcccacagatatccagaaccctctaagaagttcctgctattaaatatattaatttatcgatgaatgtggttagagacaactgaatgaccctgccgtgtaccagctgagagactctaaatccagtgacaagtctg

SEQ ID NO: 169 - DNA, 163 bp, synthetic construct
  misc_feature 1..163: note = TCGA-67
  source 1..163: mol_type = other DNA
gagatcatgtcctaaccctgatcctcttgtcccacagatatccagaaccctctaagaagttcctgctatattaaatatattaatttatcggaatgtggttagagacaactgattgaccctgccgtgtaccagctgagagactctaaatccagtgacaagtctg

SEQ ID NO: 170 - DNA, 165 bp, synthetic construct
  misc_feature 1..165: note = TCGA-68
  source 1..165: mol_type = other DNA
gagatcatgtcctaaccctgatcctcttgtcccacagatatccagaaccctctaagaagttcctgcttcgataattaatatattaataaatagaatgtggttagagacaactggatgaccctgccgtgtaccagctgagagactctaaatccagtgacaagtctg

SEQ ID NO: 171 - DNA, 165 bp, synthetic construct
  misc_feature 1..165: note = TCGA-69
  source 1..165: mol_type = other DNA
gagatcatgtcctaaccctgatcctcttgtcccacagatatccagaaccctctaagaagttcctgcttatcgataattaatatattaataaagaatgtggttagagacaactgtctgaccctgccgtgtaccagctgagagactctaaatccagtgacaagtctg

SEQ ID NO: 172 - DNA, 165 bp, synthetic construct
  misc_feature 1..165: note = TCGA-70
  source 1..165: mol_type = other DNA
gagatcatgtcctaaccctgatcctcttgtcccacagatatccagaaccctctaagaagttcctgctaatatcgataattaatatattaatagaatgtggttagagacaacttgttgaccctgccgtgtaccagctgagagactctaaatccagtgacaagtctg

SEQ ID NO: 173 - DNA, 165 bp, synthetic construct
  misc_feature 1..165: note = TCGA-71
  source 1..165: mol_type = other DNA
gagatcatgtcctaaccctgatcctcttgtcccacagatatccagaaccctctaagaagttcctgcttaaatatcgataattaatatattaagaatgtggttagagacaagaacctgaccctgccgtgtaccagctgagagactctaaatccagtgacaagtctg

SEQ ID NO: 174 - DNA, 165 bp, synthetic construct
  misc_feature 1..165: note = TCGA-72
  source 1..165: mol_type = other DNA
gagatcatgtcctaaccctgatcctcttgtcccacagatatccagaaccctctaagaagttcctgctaataaatatcgataattaatatattgaatgtggttagagacaagaacgtgaccctgccgtgtaccagctgagagactctaaatccagtgacaagtctg

SEQ ID NO: 175 - DNA, 165 bp, synthetic construct
  misc_feature 1..165: note = TCGA-73
  source 1..165: mol_type = other DNA
gagatcatgtcctaaccctgatcctcttgtcccacagatatccagaaccctctaagaagttcctgctttaataaatatcgataattaatatagaatgtggttagagacaagaagatgaccctgccgtgtaccagctgagagactctaaatccagtgacaagtctg

SEQ ID NO: 176 - DNA, 165 bp, synthetic construct
  misc_feature 1..165: note = TCGA-74
  source 1..165: mol_type = other DNA
gagatcatgtcctaaccctgatcctcttgtcccacagatatccagaaccctctaagaagttcctgcttattaataaatatcgataattaatagaatgtggttagagacaagaagttgaccctgccgtgtaccagctgagagactctaaatccagtgacaagtctg

SEQ ID NO: 177 - DNA, 165 bp, synthetic construct
  misc_feature 1..165: note = TCGA-75
  source 1..165: mol_type = other DNA
gagatcatgtcctaaccctgatcctcttgtcccacagatatccagaaccctctaagaagttcctgcttatattaataaatatcgataattaagaatgtggttagagacaagaatctgaccctgccgtgtaccagctgagagactctaaatccagtgacaagtctg

SEQ ID NO: 178 - DNA, 165 bp, synthetic construct
  misc_feature 1..165: note = TCGA-76
  source 1..165: mol_type = other DNA
gagatcatgtcctaaccctgatcctcttgtcccacagatatccagaaccctctaagaagttcctgctaatatattaataaatatcgataattgaatgtggttagagacaagactgtgaccctgccgtgtaccagctgagagactctaaatccagtgacaagtctg

SEQ ID NO: 179 - DNA, 165 bp, synthetic construct
  misc_feature 1..165: note = TCGA-77
  source 1..165: mol_type = other DNA
gagatcatgtcctaaccctgatcctcttgtcccacagatatccagaaccctctaagaagttcctgctttaatatattaataaatatcgataagaatgtggttagagacaagagaatgaccctgccgtgtaccagctgagagactctaaatccagtgacaagtctg

SEQ ID NO: 180 - DNA, 165 bp, synthetic construct
  misc_feature 1..165: note = TCGA-78
  source 1..165: mol_type = other DNA
gagatcatgtcctaaccctgatcctcttgtcccacagatatccagaaccctctaagaagttcctgctaattaatatattaataaatatcgatgaatgtggttagagacaagagagtgaccctgccgtgtaccagctgagagactctaaatccagtgacaagtctg

SEQ ID NO: 181 - DNA, 165 bp, synthetic construct
  misc_feature 1..165: note = TCGA-79
  source 1..165: mol_type = other DNA
gagatcatgtcctaaccctgatcctcttgtcccacagatatccagaaccctctaagaagttcctgctataattaatatattaataaatatcggaatgtggttagagacaagagcatgaccctgccgtgtaccagctgagagactctaaatccagtgacaagtctg

SEQ ID NO: 182 - DNA, 167 bp, synthetic construct
  misc_feature 1..167: note = TCGA-80
  source 1..167: mol_type = other DNA
gagatcatgtcctaaccctgatcctcttgtcccacagatatccagaaccctctaagaagttcctgcttcgattattaatatataaataattatagaatgtggttagagacaagagcttgaccctgccgtgtaccagctgagagactctaaatccagtgacaagtctg

SEQ ID NO: 183 - DNA, 167 bp, synthetic construct
  misc_feature 1..167: note = TCGA-81
  source 1..167: mol_type = other DNA
gagatcatgtcctaaccctgatcctcttgtcccacagatatccagaaccctctaagaagttcctgcttatcgattattaatatataaataattagaatgtggttagagacaagaggatgaccctgccgtgtaccagctgagagactctaaatccagtgacaagtctg

SEQ ID NO: 184 - DNA, 167 bp, synthetic construct
  misc_feature 1..167: note = TCGA-82
  source 1..167: mol_type = other DNA
gagatcatgtcctaaccctgatcctcttgtcccacagatatccagaaccctctaagaagttcctgcttatatcgattattaatatataaataatgaatgtggttagagacaagatactgaccctgccgtgtaccagctgagagactctaaatccagtgacaagtctg

SEQ ID NO: 185 - DNA, 167 bp, synthetic construct
  misc_feature 1..167: note = TCGA-83
  source 1..167: mol_type = other DNA
gagatcatgtcctaaccctgatcctcttgtcccacagatatccagaaccctctaagaagttcctgctattatatcgattattaatatataaatagaatgtggttagagacaagatgctgaccctgccgtgtaccagctgagagactctaaatccagtgacaagtctg

SEQ ID NO: 186 - DNA, 167 bp, synthetic construct
  misc_feature 1..167: note = TCGA-84
  source 1..167: mol_type = other DNA
gagatcatgtcctaaccctgatcctcttgtcccacagatatccagaaccctctaagaagttcctgcttaattatatcgattattaatatataaagaatgtggttagagacaagatggtgaccctgccgtgtaccagctgagagactctaaatccagtgacaagtctg

SEQ ID NO: 187 - DNA, 167 bp, synthetic construct
  misc_feature 1..167: note = TCGA-85
  source 1..167: mol_type = other DNA
gagatcatgtcctaaccctgatcctcttgtcccacagatatccagaaccctctaagaagttcctgctaataattatatcgattattaatatatagaatgtggttagagacaagcaaatgaccctgccgtgtaccagctgagagactctaaatccagtgacaagtctg

SEQ ID NO: 188 - DNA, 167 bp, synthetic construct
  misc_feature 1..167: note = TCGA-86
  source 1..167: mol_type = other DNA
gagatcatgtcctaaccctgatcctcttgtcccacagatatccagaaccctctaagaagttcctgcttaaataattatatcgattattaatatagaatgtggttagagacaagcacttgaccctgccgtgtaccagctgagagactctaaatccagtgacaagtctg

SEQ ID NO: 189 - DNA, 167 bp, synthetic construct
  misc_feature 1..167: note = TCGA-87
  source 1..167: mol_type = other DNA
gagatcatgtcctaaccctgatcctcttgtcccacagatatccagaaccctctaagaagttcctgcttataaataattatatcgattattaatagaatgtggttagagacaagcatgtgaccctgccgtgtaccagctgagagactctaaatccagtgacaagtctg

SEQ ID NO: 190 - DNA, 167 bp, synthetic construct
  misc_feature 1..167: note = TCGA-88
  source 1..167: mol_type = other DNA
gagatcatgtcctaaccctgatcctcttgtcccacagatatccagaaccctctaagaagttcctgcttatataaataattatatcgattattaagaatgtggttagagacaagcctttgaccctgccgtgtaccagctgagagactctaaatccagtgacaagtctg

SEQ ID NO: 191 - DNA, 167 bp, synthetic construct
  misc_feature 1..167: note = TCGA-89
  source 1..167: mol_type = other DNA
gagatcatgtcctaaccctgatcctcttgtcccacagatatccagaaccctctaagaagttcctgctaatatataaataattatatcgattattgaatgtggttagagacaagcgattgaccctgccgtgtaccagctgagagactctaaatccagtgacaagtctg

SEQ ID NO: 192 - DNA, 167 bp, synthetic construct
  misc_feature 1..167: note = TCGA-90
  source 1..167: mol_type = other DNA
gagatcatgtcctaaccctgatcctcttgtcccacagatatccagaaccctctaagaagttcctgctttaatatataaataattatatcgattagaatgtggttagagacaagctcatgaccctgccgtgtaccagctgagagactctaaatccagtgacaagtctg

SEQ ID NO: 193 - DNA, 167 bp, synthetic construct
  misc_feature 1..167: note = TCGA-91
  source 1..167: mol_type = other DNA
gagatcatgtcctaaccctgatcctcttgtcccacagatatccagaaccctctaagaagttcctgcttattaatatataaataattatatcgatgaatgtggttagagacaagcttatgaccctgccgtgtaccagctgagagactctaaatccagtgacaagtctg

SEQ ID NO: 194 - DNA, 167 bp, synthetic construct
  misc_feature 1..167: note = TCGA-92
  source 1..167: mol_type = other DNA
gagatcatgtcctaaccctgatcctcttgtcccacagatatccagaaccctctaagaagttcctgctattattaatatataaataattatatcggaatgtggttagagacaaggaaatgaccctgccgtgtaccagctgagagactctaaatccagtgacaagtctg

SEQ ID NO: 195 - DNA, 169 bp, synthetic construct
  misc_feature 1..169: note = TCGA-93
  source 1..169: mol_type = other DNA
gagatcatgtcctaaccctgatcctcttgtcccacagatatccagaaccctctaagaagttcctgcttcgattattaatatataaatatttaaatagaatgtggttagagacaaggaagtgaccctgccgtgtaccagctgagagactctaaatccagtgacaagtctg

SEQ ID NO: 196 - DNA, 169 bp, synthetic construct
  misc_feature 1..169: note = TCGA-94
  source 1..169: mol_type = other DNA
gagatcatgtcctaaccctgatcctcttgtcccacagatatccagaaccctctaagaagttcctgcttatcgattattaatatataaatatttaaagaatgtggttagagacaagggaatgaccctgccgtgtaccagctgagagactctaaatccagtgacaagtctg

SEQ ID NO: 197 - DNA, 169 bp, synthetic construct
  misc_feature 1..169: note = TCGA-95
  source 1..169: mol_type = other DNA
gagatcatgtcctaaccctgatcctcttgtcccacagatatccagaaccctctaagaagttcctgctaatatcgattattaatatataaatatttagaatgtggttagagacaaggtactgaccctgccgtgtaccagctgagagactctaaatccagtgacaagtctg

SEQ ID NO: 198 - DNA, 169 bp, synthetic construct
  misc_feature 1..169: note = TCGA-96
  source 1..169: mol_type = other DNA
gagatcatgtcctaaccctgatcctcttgtcccacagatatccagaaccctctaagaagttcctgcttaaatatcgattattaatatataaatattgaatgtggttagagacaaggttatgaccctgccgtgtaccagctgagagactctaaatccagtgacaagtctg

SEQ ID NO: 199 - DNA, 169 bp, synthetic construct
  misc_feature 1..169: note = TCGA-97
  source 1..169: mol_type = other DNA
gagatcatgtcctaaccctgatcctcttgtcccacagatatccagaaccctctaagaagttcctgcttttaaatatcgattattaatatataaatagaatgtggttagagacaaggttgtgaccctgccgtgtaccagctgagagactctaaatccagtgacaagtctg

SEQ ID NO: 200 - DNA, 169 bp, synthetic construct
  misc_feature 1..169: note = TCGA-98
  source 1..169: mol_type = other DNA
gagatcatgtcctaaccctgatcctcttgtcccacagatatccagaaccctctaagaagttcctgcttatttaaatatcgattattaatatataaagaatgtggttagagacaagtaactgaccctgccgtgtaccagctgagagactctaaatccagtgacaagtctg

SEQ ID NO: 201 - DNA, 169 bp, synthetic construct
  misc_feature 1..169: note = TCGA-99
  source 1..169: mol_type = other DNA
gagatcatgtcctaaccctgatcctcttgtcccacagatatccagaaccctctaagaagttcctgctaatatttaaatatcgattattaatatatagaatgtggttagagacaagtacatgaccctgccgtgtaccagctgagagactctaaatccagtgacaagtctg

SEQ ID NO: 202 - DNA, 169 bp, synthetic construct
  misc_feature 1..169: note = TCGA-100
  source 1..169: mol_type = other DNA
gagatcatgtcctaaccctgatcctcttgtcccacagatatccagaaccctctaagaagttcctgcttaaatatttaaatatcgattattaatatagaatgtggttagagacaagtacgtgaccctgccgtgtaccagctgagagactctaaatccagtgacaagtctg

SEQ ID NO: 203 - DNA, 169 bp, synthetic construct
  misc_feature 1..169: note = TCGA-101
  source 1..169: mol_type = other DNA
gagatcatgtcctaaccctgatcctcttgtcccacagatatccagaaccctctaagaagttcctgcttataaatatttaaatatcgattattaatagaatgtggttagagacaagtagctgaccctgccgtgtaccagctgagagactctaaatccagtgacaagtctg

SEQ ID NO: 204 - DNA, 169 bp, synthetic construct
  misc_feature 1..169: note = TCGA-102
  source 1..169: mol_type = other DNA
gagatcatgtcctaaccctgatcctcttgtcccacagatatccagaaccctctaagaagttcctgcttatataaatatttaaatatcgattattaagaatgtggttagagacaagtctctgaccctgccgtgtaccagctgagagactctaaatccagtgacaagtctg

SEQ ID NO: 205 - DNA, 169 bp, synthetic construct
  misc_feature 1..169: note = TCGA-103
  source 1..169: mol_type = other DNA
gagatcatgtcctaaccctgatcctcttgtcccacagatatccagaaccctctaagaagttcctgctaatatataaatatttaaatatcgattattgaatgtggttagagacaagtctgtgaccctgccgtgtaccagctgagagactctaaatccagtgacaagtctg

SEQ ID NO: 206 - DNA, 169 bp, synthetic construct
  misc_feature 1..169: note = TCGA-104
  source 1..169: mol_type = other DNA
gagatcatgtcctaaccctgatcctcttgtcccacagatatccagaaccctctaagaagttcctgctttaatatataaatatttaaatatcgattagaatgtggttagagacaagtgtgtgaccctgccgtgtaccagctgagagactctaaatccagtgacaagtctg

SEQ ID NO: 207 - DNA, 169 bp, synthetic construct
  misc_feature 1..169: note = TCGA-105
  source 1..169: mol_type = other DNA
gagatcatgtcctaaccctgatcctcttgtcccacagatatccagaaccctctaagaagttcctgcttattaatatataaatatttaaatatcgatgaatgtggttagagacaagttagtgaccctgccgtgtaccagctgagagactctaaatccagtgacaagtctg

SEQ ID NO: 208 - DNA, 169 bp, synthetic construct
  misc_feature 1..169: note = TCGA-106
  source 1..169: mol_type = other DNA
gagatcatgtcctaaccctgatcctcttgtcccacagatatccagaaccctctaagaagttcctgctattattaatatataaatatttaaatatcggaatgtggttagagacaagttcctgaccctgccgtgtaccagctgagagactctaaatccagtgacaagtctg

SEQ ID NO: 209 - DNA, 171 bp, synthetic construct
  misc_feature 1..171: note = TCGA-107
  source 1..171: mol_type = other DNA
gagatcatgtcctaaccctgatcctcttgtcccacagatatccagaaccctctaagaagttcctgcttcgattattaattaataaatatttaaatatagaatgtggttagagacaagttcttgaccctgccgtgtaccagctgagagactctaaatccagtgacaagtctg

SEQ ID NO: 210 - DNA, 171 bp, synthetic construct
  misc_feature 1..171: note = TCGA-108
  source 1..171: mol_type = other DNA
gagatcatgtcctaaccctgatcctcttgtcccacagatatccagaaccctctaagaagttcctgcttatcgattattaattaataaatatttaaatagaatgtggttagagacaataagctgaccctgccgtgtaccagctgagagactctaaatccagtgacaagtctg

SEQ ID NO: 211 - DNA, 171 bp, synthetic construct
  misc_feature 1..171: note = TCGA-109
  source 1..171: mol_type = other DNA
gagatcatgtcctaaccctgatcctcttgtcccacagatatccagaaccctctaagaagttcctgcttatatcgattattaattaataaatatttaaagaatgtggttagagacaatactctgaccctgccgtgtaccagctgagagactctaaatccagtgacaagtctg

SEQ ID NO: 212 - DNA, 171 bp, synthetic construct
  misc_feature 1..171: note = TCGA-110
  source 1..171: mol_type = other DNA
gagatcatgtcctaaccctgatcctcttgtcccacagatatccagaaccctctaagaagttcctgctaatatatcgattattaattaataaatatttagaatgtggttagagacaatcacatgaccctgccgtgtaccagctgagagactctaaatccagtgacaagtctg

SEQ ID NO: 213 - DNA, 171 bp, synthetic construct
  misc_feature 1..171: note = TCGA-111
  source 1..171: mol_type = other DNA
gagatcatgtcctaaccctgatcctcttgtcccacagatatccagaaccctctaagaagttcctgcttaaatatatcgattattaattaataaatattgaatgtggttagagacaatcatctgaccctgccgtgtaccagctgagagactctaaatccagtgacaagtctg

SEQ ID NO: 214 - DNA, 171 bp, synthetic construct
  misc_feature 1..171: note = TCGA-112
  source 1..171: mol_type = other DNA
gagatcatgtcctaaccctgatcctcttgtcccacagatatccagaaccctctaagaagttcctgcttttaaatatatcgattattaattaataaatagaatgtggttagagacaatcccttgaccctgccgtgtaccagctgagagactctaaatccagtgacaagtctg

SEQ ID NO: 215 - DNA, 171 bp, synthetic construct
  misc_feature 1..171: note = TCGA-113
  source 1..171: mol_type = other DNA
gagatcatgtcctaaccctgatcctcttgtcccacagatatccagaaccctctaagaagttcctgcttatttaaatatatcgattattaattaataaagaatgtggttagagacaatccgatgaccctgccgtgtaccagctgagagactctaaatccagtgacaagtctg

SEQ ID NO: 216 - DNA, 171 bp, synthetic construct
  misc_feature 1..171: note = TCGA-114
  source 1..171: mol_type = other DNA
gagatcatgtcctaaccctgatcctcttgtcccacagatatccagaaccctctaagaagttcctgctaatatttaaatatatcgattattaattaatagaatgtggttagagacaatcgaatgaccctgccgtgtaccagctgagagactctaaatccagtgacaagtctg

SEQ ID NO: 217 - DNA, 171 bp, synthetic construct
  misc_feature 1..171: note = TCGA-115
  source 1..171: mol_type = other DNA
gagatcatgtcctaaccctgatcctcttgtcccacagatatccagaaccctctaagaagttcctgcttaaatatttaaatatatcgattattaattaagaatgtggttagagacaatcggatgaccctgccgtgtaccagctgagagactctaaatccagtgacaagtctg

SEQ ID NO: 218 - DNA, 171 bp, synthetic construct
  misc_feature 1..171: note = TCGA-116
  source 1..171: mol_type = other DNA
gagatcatgtcctaaccctgatcctcttgtcccacagatatccagaaccctctaagaagttcctgctaataaatatttaaatatatcgattattaattgaatgtggttagagacaatcggttgaccctgccgtgtaccagctgagagactctaaatccagtgacaagtctg

SEQ ID NO: 219 - DNA, 171 bp, synthetic construct
  misc_feature 1..171: note = TCGA-117
  source 1..171: mol_type = other DNA
gagatcatgtcctaaccctgatcctcttgtcccacagatatccagaaccctctaagaagttcctgctttaataaatatttaaatatatcgattattaagaatgtggttagagacaatcgtctgaccctgccgtgtaccagctgagagactctaaatccagtgacaagtctg

SEQ ID NO: 220 - DNA, 171 bp, synthetic construct
  misc_feature 1..171: note = TCGA-118
  source 1..171: mol_type = other DNA
gagatcatgtcctaaccctgatcctcttgtcccacagatatccagaaccctctaagaagttcctgctaattaataaatatttaaatatatcgattattgaatgtggttagagacaatcgtgtgaccctgccgtgtaccagctgagagactctaaatccagtgacaagtctg

SEQ ID NO: 221 - DNA, 171 bp, synthetic construct
  misc_feature 1..171: note = TCGA-119
  source 1..171: mol_type = other DNA
gagatcatgtcctaaccctgatcctcttgtcccacagatatccagaaccctctaagaagttcctgctttaattaataaatatttaaatatatcgattagaatgtggttagagacaatgacctgaccctgccgtgtaccagctgagagactctaaatccagtgacaagtctg

SEQ ID NO: 222 - DNA, 171 bp, synthetic construct
  misc_feature 1..171: note = TCGA-120
  source 1..171: mol_type = other DNA
gagatcatgtcctaaccctgatcctcttgtcccacagatatccagaaccctctaagaagttcctgcttattaattaataaatatttaaatatatcgatgaatgtggttagagacaatgacgtgaccctgccgtgtaccagctgagagactctaaatccagtgacaagtctg

SEQ ID NO: 223 - DNA, 171 bp, synthetic construct
  misc_feature 1..171: note = TCGA-121
  source 1..171: mol_type = other DNA
gagatcatgtcctaaccctgatcctcttgtcccacagatatccagaaccctctaagaagttcctgctattattaattaataaatatttaaatatatcggaatgtggttagagacaatgccatgaccctgccgtgtaccagctgagagactctaaatccagtgacaagtctg

SEQ ID NO: 224 - DNA, 173 bp, synthetic construct
  misc_feature 1..173: note = TCGA-122
  source 1..173: mol_type = other DNA
gagatcatgtcctaaccctgatcctcttgtcccacagatatccagaaccctctaagaagttcctgcttcgattattaattaataaatatttatttaaatagaatgtggttagagacaatgctatgaccctgccgtgtaccagctgagagactctaaatccagtgacaagtctg

SEQ ID NO: 225 - DNA, 173 bp, synthetic construct
  misc_feature 1..173: note = TCGA-123
  source 1..173: mol_type = other DNA
gagatcatgtcctaaccctgatcctcttgtcccacagatatccagaaccctctaagaagttcctgcttatcgattattaattaataaatatttatttaaagaatgtggttagagacaatgctttgaccctgccgtgtaccagctgagagactctaaatccagtgacaagtctg

SEQ ID NO: 226 - DNA, 173 bp, synthetic construct
  misc_feature 1..173: note = TCGA-124
  source 1..173: mol_type = other DNA
gagatcatgtcctaaccctgatcctcttgtcccacagatatccagaaccctctaagaagttcctgctaatatcgattattaattaataaatatttatttagaatgtggttagagacaatggactgaccctgccgtgtaccagctgagagactctaaatccagtgacaagtctg

SEQ ID NO: 227 - DNA, 173 bp, synthetic construct
  misc_feature 1..173: note = TCGA-125
  source 1..173: mol_type = other DNA
gagatcatgtcctaaccctgatcctcttgtcccacagatatccagaaccctctaagaagttcctgcttaaatatcgattattaattaataaatatttattgaatgtggttagagacaattcactgaccctgccgtgtaccagctgagagactctaaatccagtgacaagtctg

SEQ ID NO: 228 - DNA, 173 bp, synthetic construct
  misc_feature 1..173: note = TCGA-126
  source 1..173: mol_type = other DNA
gagatcatgtcctaaccctgatcctcttgtcccacagatatccagaaccctctaagaagttcctgcttttaaatatcgattattaattaataaatatttagaatgtggttagagacaattccgtgaccctgccgtgtaccagctgagagactctaaatccagtgacaagtctg

SEQ ID NO: 229 - DNA, 173 bp, synthetic construct
  misc_feature 1..173: note = TCGA-127
  source 1..173: mol_type = other DNA
gagatcatgtcctaaccctgatcctcttgtcccacagatatccagaaccctctaagaagttcctgcttatttaaatatcgattattaattaataaatattgaatgtggttagagacaattcgatgaccctgccgtgtaccagctgagagactctaaatccagtgacaagtctg

SEQ ID NO: 230 - DNA, 173 bp, synthetic construct
  misc_feature 1..173: note = TCGA-128
  source 1..173: mol_type = other DNA
gagatcatgtcctaaccctgatcctcttgtcccacagatatccagaaccctctaagaagttcctgcttttatttaaatatcgattattaattaataaatagaatgtggttagagacaattgcatgaccctgccgtgtaccagctgagagactctaaatccagtgacaagtctg

SEQ ID NO: 231 - DNA, 173 bp, synthetic construct
  misc_feature 1..173: note = TCGA-129
  source 1..173: mol_type = other DNA
gagatcatgtcctaaccctgatcctcttgtcccacagatatccagaaccctctaagaagttcctgcttatttatttaaatatcgattattaattaataaagaatgtggttagagacaattgcttgaccctgccgtgtaccagctgagagactctaaatccagtgacaagtctg

SEQ ID NO: 232 - DNA, 173 bp, synthetic construct
  misc_feature 1..173: note = TCGA-130
  source 1..173: mol_type = other DNA
gagatcatgtcctaaccctgatcctcttgtcccacagatatccagaaccctctaagaagttcctgctaatatttatttaaatatcgattattaattaatagaatgtggttagagacacaaatgtgaccctgccgtgtaccagctgagagactctaaatccagtgacaagtctg

SEQ ID NO: 233 - DNA, 173 bp, synthetic construct
  misc_feature 1..173: note = TCGA-131
  source 1..173: mol_type = other DNA
gagatcatgtcctaaccctgatcctcttgtcccacagatatccagaaccctctaagaagttcctgcttaaatatttatttaaatatcgattattaattaagaatgtggttagagacacaactttgaccctgccgtgtaccagctgagagactctaaatccagtgacaagtctg

SEQ ID NO: 234 - DNA, 173 bp, synthetic construct
  misc_feature 1..173: note = TCGA-132
  source 1..173: mol_type = other DNA
gagatcatgtcctaaccctgatcctcttgtcccacagatatccagaaccctctaagaagttcctgctaataaatatttatttaaatatcgattattaattgaatgtggttagagacacaagattgaccctgccgtgtaccagctgagagactctaaatccagtgacaagtctg

SEQ ID NO: 235 - DNA, 173 bp, synthetic construct
  misc_feature 1..173: note = TCGA-133
  source 1..173: mol_type = other DNA
gagatcatgtcctaaccctgatcctcttgtcccacagatatccagaaccctctaagaagttcctgctttaataaatatttatttaaatatcgattattaagaatgtggttagagacacaaggttgaccctgccgtgtaccagctgagagactctaaatccagtgacaagtctg

SEQ ID NO: 236 - DNA, 173 bp, synthetic construct
  misc_feature 1..173: note = TCGA-134
  source 1..173: mol_type = other DNA
gagatcatgtcctaaccctgatcctcttgtcccacagatatccagaaccctctaagaagttcctgctaattaataaatatttatttaaatatcgattattgaatgtggttagagacacaattgtgaccctgccgtgtaccagctgagagactctaaatccagtgacaagtctg

SEQ ID NO: 237 - DNA, 173 bp, synthetic construct
  misc_feature 1..173: note = TCGA-135
  source 1..173: mol_type = other DNA
gagatcatgtcctaaccctgatcctcttgtcccacagatatccagaaccctctaagaagttcctgctttaattaataaatatttatttaaatatcgattagaatgtggttagagacacacacttgaccctgccgtgtaccagctgagagactctaaatccagtgacaagtctg

SEQ ID NO: 238 - DNA, 173 bp, synthetic construct
  misc_feature 1..173: note = TCGA-136
  source 1..173: mol_type = other DNA
gagatcatgtcctaaccctgatcctcttgtcccacagatatccagaaccctctaagaagttcctgcttattaattaataaatatttatttaaatatcgatgaatgtggttagagacacacatatgaccctgccgtgtaccagctgagagactctaaatccagtgacaagtctg

SEQ ID NO: 239 - DNA, 173 bp, synthetic construct
  misc_feature 1..173: note = TCGA-137
  source 1..173: mol_type = other DNA
gagatcatgtcctaaccctgatcctcttgtcccacagatatccagaaccctctaagaagttcctgctattattaattaataaatatttatttaaatatcggaatgtggttagagacacacgtatgaccctgccgtgtaccagctgagagactctaaatccagtgacaagtctg

SEQ ID NO: 240 - DNA, 175 bp, synthetic construct
  misc_feature 1..175: note = TCGA-138
  source 1..175: mol_type = other DNA
gagatcatgtcctaaccctgatcctcttgtcccacagatatccagaaccctctaagaagttcctgcttcgattattaatattaataaatatttatttaaatagaatgtggttagagacacactaatgaccctgccgtgtaccagctgagagactctaaatccagtgacaagtctg

SEQ ID NO: 241 - DNA, 175 bp, synthetic construct
  misc_feature 1..175: note = TCGA-139
  source 1..175: mol_type = other DNA
gagatcatgtcctaaccctgatcctcttgtcccacagatatccagaaccctctaagaagttcctgcttatcgattattaatattaataaatatttatttaaagaatgtggttagagacacactagtgaccctgccgtgtaccagctgagagactctaaatccagtgacaagtctg

SEQ ID NO: 242 - DNA, 175 bp, synthetic construct
  misc_feature 1..175: note = TCGA-140
  source 1..175: mol_type = other DNA
gagatcatgtcctaaccctgatcctcttgtcccacagatatccagaaccctctaagaagttcctgctaatatcgattattaatattaataaatatttatttagaatgtggttagagacacactcttgaccctgccgtgtaccagctgagagactctaaatccagtgacaagtctg

SEQ ID NO: 243 - DNA, 175 bp, synthetic construct
  misc_feature 1..175: note = TCGA-141
  source 1..175: mol_type = other DNA
gagatcatgtcctaaccctgatcctcttgtcccacagatatccagaaccctctaagaagttcctgcttaaatatcgattattaatattaataaatatttattgaatgtggttagagacacagcaatgaccctgccgtgtaccagctgagagactctaaatccagtgacaagtctg

SEQ ID NO: 244 - DNA, 175 bp, synthetic construct
  misc_feature 1..175: note = TCGA-142
  source 1..175: mol_type = other DNA
gagatcatgtcctaaccctgatcctcttgtcccacagatatccagaaccctctaagaagttcctgcttttaaatatcgattattaatattaataaatatttagaatgtggttagagacacagcattgaccctgccgtgtaccagctgagagactctaaatccagtgacaagtctg

SEQ ID NO: 245 - DNA, 175 bp, synthetic construct
  misc_feature 1..175: note = TCGA-143
  source 1..175: mol_type = other DNA
gagatcatgtcctaaccctgatcctcttgtcccacagatatccagaaccctctaagaagttcctgcttatttaaatatcgattattaatattaataaatattgaatgtggttagagacacagtattgaccctgccgtgtaccagctgagagactctaaatccagtgacaagtctg

SEQ ID NO: 246 - DNA, 175 bp, synthetic construct
  misc_feature 1..175: note = TCGA-144
  source 1..175: mol_type = other DNA
gagatcatgtcctaaccctgatcctcttgtcccacagatatccagaaccctctaagaagttcctgcttttatttaaatatcgattattaatattaataaatagaatgtggttagagacacagtcatgaccctgccgtgtaccagctgagagactctaaatccagtgacaagtctg

SEQ ID NO: 247 - DNA, 175 bp, synthetic construct
  misc_feature 1..175: note = TCGA-145
  source 1..175: mol_type = other DNA
gagatcatgtcctaaccctgatcctcttgtcccacagatatccagaaccctctaagaagttcctgcttatttatttaaatatcgattattaatattaataaagaatgtggttagagacacagtgatgaccctgccgtgtaccagctgagagactctaaatccagtgacaagtctg

SEQ ID NO: 248 - DNA, 175 bp, synthetic construct
  misc_feature 1..175: note = TCGA-146
  source 1..175: mol_type = other DNA
gagatcatgtcctaaccctgatcctcttgtcccacagatatccagaaccctctaagaagttcctgctaatatttatttaaatatcgattattaatattaatagaatgtggttagagacacagtgttgaccctgccgtgtaccagctgagagactctaaatccagtgacaagtctg

SEQ ID NO: 249 - DNA, 175 bp, synthetic construct
  misc_feature 1..175: note = TCGA-147
  source 1..175: mol_type = other DNA
gagatcatgtcctaaccctgatcctcttgtcccacagatatccagaaccctctaagaagttcctgcttaaatatttatttaaatatcgattattaatattaagaatgtggttagagacacagttctgaccctgccgtgtaccagctgagagactctaaatccagtgacaagtctg

SEQ ID NO: 250 - DNA, 175 bp, synthetic construct
  misc_feature 1..175: note = TCGA-148
  source 1..175: mol_type = other DNA
gagatcatgtcctaaccctgatcctcttgtcccacagatatccagaaccctctaagaagttcctgctaataaatatttatttaaatatcgattattaatattgaatgtggttagagacacataagtgaccctgccgtgtaccagctgagagactctaaatccagtgacaagtctg

SEQ ID NO: 251 - DNA, 175 bp, synthetic construct
  misc_feature 1..175: note = TCGA-149
  source 1..175: mol_type = other DNA
gagatcatgtcctaaccctgatcctcttgtcccacagatatccagaaccctctaagaagttcctgctttaataaatatttatttaaatatcgattattaatagaatgtggttagagacacatatgtgaccctgccgtgtaccagctgagagactctaaatccagtgacaagtctg

SEQ ID NO: 252 - DNA, 175 bp, synthetic construct
  misc_feature 1..175: note = TCGA-150
  source 1..175: mol_type = other DNA
gagatcatgtcctaaccctgatcctcttgtcccacagatatccagaaccctctaagaagttcctgcttattaataaatatttatttaaatatcgattattaagaatgtggttagagacacatcactgaccctgccgtgtaccagctgagagactctaaatccagtgacaagtctg

SEQ ID NO: 253 - DNA, 175 bp, synthetic construct
  misc_feature 1..175: note = TCGA-151
  source 1..175: mol_type = other DNA
gagatcatgtcctaaccctgatcctcttgtcccacagatatccagaaccctctaagaagttcctgctaatattaataaatatttatttaaatatcgattattgaatgtggttagagacacatccttgaccctgccgtgtaccagctgagagactctaaatccagtgacaagtctg

SEQ ID NO: 254 - DNA, 175 bp, synthetic construct
  misc_feature 1..175: note = TCGA-152
  source 1..175: mol_type = other DNA
gagatcatgtcctaaccctgatcctcttgtcccacagatatccagaaccctctaagaagttcctgctttaatattaataaatatttatttaaatatcgattagaatgtggttagagacacatcgttgaccctgccgtgtaccagctgagagactctaaatccagtgacaagtctg

SEQ ID NO: 255 - DNA, 175 bp, synthetic construct
  misc_feature 1..175: note = TCGA-153
  source 1..175: mol_type = other DNA
gagatcatgtcctaaccctgatcctcttgtcccacagatatccagaaccctctaagaagttcctgcttattaatattaataaatatttatttaaatatcgatgaatgtggttagagacacatgactgaccctgccgtgtaccagctgagagactctaaatccagtgacaagtctg

SEQ ID NO: 256 - DNA, 175 bp, synthetic construct
  misc_feature 1..175: note = TCGA-154
  source 1..175: mol_type = other DNA
gagatcatgtcctaaccctgatcctcttgtcccacagatatccagaaccctctaagaagttcctgctattattaatattaataaatatttatttaaatatcggaatgtggttagagacacatgtatgaccctgccgtgtaccagctgagagactctaaatccagtgacaagtctg

SEQ ID NO: 257 - DNA, 177 bp, synthetic construct
  misc_feature 1..177: note = TCGA-155
  source 1..177: mol_type = other DNA
gagatcatgtcctaaccctgatcctcttgtcccacagatatccagaaccctctaagaagttcctgcttcgattaattaatattaataaatatttatttataatagaatgtggttagagacacatgtgtgaccctgccgtgtaccagctgagagactctaaatccagtgacaagtctg

SEQ ID NO: 258 - DNA, 177 bp, synthetic construct
  misc_feature 1..177: note = TCGA-156
  source 1..177: mol_type = other DNA
gagatcatgtcctaaccctgatcctcttgtcccacagatatccagaaccctctaagaagttcctgcttatcgattaattaatattaataaatatttatttataagaatgtggttagagacacattcttgaccctgccgtgtaccagctgagagactctaaatccagtgacaagtctg

SEQ ID NO: 259 - DNA, 177 bp, synthetic construct
  misc_feature 1..177: note = TCGA-157
  source 1..177: mol_type = other DNA
gagatcatgtcctaaccctgatcctcttgtcccacagatatccagaaccctctaagaagttcctgctaatatcgattaattaatattaataaatatttatttatgaatgtggttagagacaccaaactgaccctgccgtgtaccagctgagagactctaaatccagtgacaagtctg

SEQ ID NO: 260 - DNA, 177 bp, synthetic construct
  misc_feature 1..177: note = TCGA-158
  source 1..177: mol_type = other DNA
gagatcatgtcctaaccctgatcctcttgtcccacagatatccagaaccctctaagaagttcctgctataatatcgattaattaatattaataaatatttatttgaatgtggttagagacaccaaagtgaccctgccgtgtaccagctgagagactctaaatccagtgacaagtctg

SEQ ID NO: 261 - DNA, 177 bp, synthetic construct
  misc_feature 1..177: note = TCGA-159
  source 1..177: mol_type = other DNA
gagatcatgtcctaaccctgatcctcttgtcccacagatatccagaaccctctaagaagttcctgctttataatatcgattaattaatattaataaatatttatgaatgtggttagagacaccaatctgaccctgccgtgtaccagctgagagactctaaatccagtgacaagtctg

SEQ ID NO: 262 - DNA, 177 bp, synthetic construct
  misc_feature 1..177: note = TCGA-160
  source 1..177: mol_type = other DNA
gagatcatgtcctaaccctgatcctcttgtcccacagatatccagaaccctctaagaagttcctgctatttataatatcgattaattaatattaataaatatttgaatgtggttagagacaccacattgaccctgccgtgtaccagctgagagactctaaatccagtgacaagtctg

SEQ ID NO: 263 - DNA, 177 bp, synthetic construct
  misc_feature 1..177: note = TCGA-161
  source 1..177: mol_type = other DNA
gagatcatgtcctaaccctgatcctcttgtcccacagatatccagaaccctctaagaagttcctgctttatttataatatcgattaattaatattaataaatatgaatgtggttagagacaccatattgaccctgccgtgtaccagctgagagactctaaatccagtgacaagtctg

SEQ ID NO: 264 - DNA, 177 bp, synthetic construct
  misc_feature 1..177: note = TCGA-162
  source 1..177: mol_type = other DNA
gagatcatgtcctaaccctgatcctcttgtcccacagatatccagaaccctctaagaagttcctgctatttatttataatatcgattaattaatattaataaatgaatgtggttagagacacctcattgaccctgccgtgtaccagctgagagactctaaatccagtgacaagtctg

SEQ ID NO: 265 - DNA, 177 bp, synthetic construct
  misc_feature 1..177: note = TCGA-163
  source 1..177: mol_type = other DNA
gagatcatgtcctaaccctgatcctcttgtcccacagatatccagaaccctctaagaagttcctgctatatttatttataatatcgattaattaatattaataagaatgtggttagagacaccttcatgaccctgccgtgtaccagctgagagactctaaatccagtgacaagtctg

SEQ ID NO: 266 - DNA, 177 bp, synthetic construct
  misc_feature 1..177: note = TCGA-164
  source 1..177: mol_type = other DNA
gagatcatgtcctaaccctgatcctcttgtcccacagatatccagaaccctctaagaagttcctgctaaatatttatttataatatcgattaattaatattaatgaatgtggttagagacaccttgatgaccctgccgtgtaccagctgagagactctaaatccagtgacaagtctg

SEQ ID NO: 267 - DNA, 177 bp, synthetic construct
  misc_feature 1..177: note = TCGA-165
  source 1..177: mol_type = other DNA
gagatcatgtcctaaccctgatcctcttgtcccacagatatccagaaccctctaagaagttcctgctataaatatttatttataatatcgattaattaatattagaatgtggttagagacacctttctgaccctgccgtgtaccagctgagagactctaaatccagtgacaagtctg

SEQ ID NO: 268 - DNA, 177 bp, synthetic construct
  misc_feature 1..177: note = TCGA-166
  source 1..177: mol_type = other DNA
gagatcatgtcctaaccctgatcctcttgtcccacagatatccagaaccctctaagaagttcctgcttaataaatatttatttataatatcgattaattaatatgaatgtggttagagacacgaaagtgaccctgccgtgtaccagctgagagactctaaatccagtgacaagtctg

SEQ ID NO: 269 - DNA, 177 bp, synthetic construct
  misc_feature 1..177: note = TCGA-167
  source 1..177: mol_type = other DNA
gagatcatgtcctaaccctgatcctcttgtcccacagatatccagaaccctctaagaagttcctgctattaataaatatttatttataatatcgattaattaatgaatgtggttagagacacgactatgaccctgccgtgtaccagctgagagactctaaatccagtgacaagtctg

SEQ ID NO: 270 - DNA, 177 bp, synthetic construct
  misc_feature 1..177: note = TCGA-168
  source 1..177: mol_type = other DNA
gagatcatgtcctaaccctgatcctcttgtcccacagatatccagaaccctctaagaagttcctgctatattaataaatatttatttataatatcgattaattagaatgtggttagagacacgagtatgaccctgccgtgtaccagctgagagactctaaatccagtgacaagtctg

SEQ ID NO: 271 - DNA, 177 bp, synthetic construct
  misc_feature 1..177: note = TCGA-169
  source 1..177: mol_type = other DNA
gagatcatgtcctaaccctgatcctcttgtcccacagatatccagaaccctctaagaagttcctgcttaatattaataaatatttatttataatatcgattaatgaatgtggttagagacacgatcttgaccctgccgtgtaccagctgagagactctaaatccagtgacaagtctg

SEQ ID NO: 272 - DNA, 177 bp, synthetic construct
  misc_feature 1..177: note = TCGA-170
  source 1..177: mol_type = other DNA
gagatcatgtcctaaccctgatcctcttgtcccacagatatccagaaccctctaagaagttcctgctattaatattaataaatatttatttataatatcgattagaatgtggttagagacacgatgttgaccctgccgtgtaccagctgagagactctaaatccagtgacaagtctg

SEQ ID NO: 273 - DNA, 177 bp, synthetic construct
  misc_feature 1..177: note = TCGA-171
  source 1..177: mol_type = other DNA
gagatcatgtcctaaccctgatcctcttgtcccacagatatccagaaccctctaagaagttcctgcttaattaatattaataaatatttatttataatatcgatgaatgtggttagagacacggtattgaccctgccgtgtaccagctgagagactctaaatccagtgacaagtctg

SEQ ID NO: 274 - DNA, 177 bp, synthetic construct
  misc_feature 1..177: note = TCGA-172
  source 1..177: mol_type = other DNA
gagatcatgtcctaaccctgatcctcttgtcccacagatatccagaaccctctaagaagttcctgctattaattaatattaataaatatttatttataatatcggaatgtggttagagacacggttttgaccctgccgtgtaccagctgagagactctaaatccagtgacaagtctg

SEQ ID NO: 275 - DNA, 179 bp, synthetic construct
  misc_feature 1..179: note = TCGA-173
  source 1..179: mol_type = other DNA
gagatcatgtcctaaccctgatcctcttgtcccacagatatccagaaccctctaagaagttcctgcttcgattaattaatattaatttaaatatttatttatataagaatgtggttagagacacgtaattgaccctgccgtgtaccagctgagagactctaaatccagtgacaagtctg

SEQ ID NO: 276 - DNA, 179 bp, synthetic construct
  misc_feature 1..179: note = TCGA-174
  source 1..179: mol_type = other DNA
gagatcatgtcctaaccctgatcctcttgtcccacagatatccagaaccctctaagaagttcctgctaatcgattaattaatattaatttaaatatttatttatatgaatgtggttagagacacgtatttgaccctgccgtgtaccagctgagagactctaaatccagtgacaagtctg

SEQ ID NO: 277 - DNA, 179 bp, synthetic construct
  misc_feature 1..179: note = TCGA-175
  source 1..179: mol_type = other DNA
gagatcatgtcctaaccctgatcctcttgtcccacagatatccagaaccctctaagaagttcctgctataatcgattaattaatattaatttaaatatttatttatgaatgtggttagagacacgtgaatgaccctgccgtgtaccagctgagagactctaaatccagtgacaagtctg

SEQ ID NO: 278 - DNA, 179 bp, synthetic construct
  misc_feature 1..179: note = TCGA-176
  source 1..179: mol_type = other DNA
gagatcatgtcctaaccctgatcctcttgtcccacagatatccagaaccctctaagaagttcctgctatataatcgattaattaatattaatttaaatatttatttgaatgtggttagagacacgtgtttgaccctgccgtgtaccagctgagagactctaaatccagtgacaagtctg

SEQ ID NO: 279 - DNA, 179 bp, synthetic construct
  misc_feature 1..179: note = TCGA-177
  source 1..179: mol_type = other DNA
gagatcatgtcctaaccctgatcctcttgtcccacagatatccagaaccctctaagaagttcctgctttatataatcgattaattaatattaatttaaatatttatgaatgtggttagagacacgttgatgaccctgccgtgtaccagctgagagactctaaatccagtgacaagtctg

SEQ ID NO: 280 - DNA, 179 bp, synthetic construct
  misc_feature 1..179: note = TCGA-178
  source 1..179: mol_type = other DNA
gagatcatgtcctaaccctgatcctcttgtcccacagatatccagaaccctctaagaagttcctgctatttatataatcgattaattaatattaatttaaatatttgaatgtggttagagacacgttgttgaccctgccgtgtaccagctgagagactctaaatccagtgacaagtctg

SEQ ID NO: 281 - DNA, 179 bp, synthetic construct
  misc_feature 1..179: note = TCGA-179
  source 1..179: mol_type = other DNA
gagatcatgtcctaaccctgatcctcttgtcccacagatatccagaaccctctaagaagttcctgctttatttatataatcgattaattaatattaatttaaatatgaatgtggttagagacacgtttctgaccctgccgtgtaccagctgagagactctaaatccagtgacaagtctg

SEQ ID NO: 282 - DNA, 179 bp, synthetic construct
  misc_feature 1..179: note = TCGA-180
  source 1..179: mol_type = other DNA
gagatcatgtcctaaccctgatcctcttgtcccacagatatccagaaccctctaagaagttcctgctatttatttatataatcgattaattaatattaatttaaatgaatgtggttagagacactaacctgaccctgccgtgtaccagctgagagactctaaatccagtgacaagtctg

SEQ ID NO: 283 - DNA, 179 bp, synthetic construct
  misc_feature 1..179: note = TCGA-181
  source 1..179: mol_type = other DNA
gagatcatgtcctaaccctgatcctcttgtcccacagatatccagaaccctctaagaagttcctgctatatttatttatataatcgattaattaatattaatttaagaatgtggttagagacactaacgtgaccctgccgtgtaccagctgagagactctaaatccagtgacaagtctg

SEQ ID NO: 284 - DNA, 179 bp, synthetic construct
  misc_feature 1..179: note = TCGA-182
  source 1..179: mol_type = other DNA
gagatcatgtcctaaccctgatcctcttgtcccacagatatccagaaccctctaagaagttcctgctaaatatttatttatataatcgattaattaatattaatttgaatgtggttagagacactactgtgaccctgccgtgtaccagctgagagactctaaatccagtgacaagtctg

SEQ ID NO: 285 - DNA, 179 bp, synthetic construct
  misc_feature 1..179: note = TCGA-183
  source 1..179: mol_type = other DNA
gagatcatgtcctaaccctgatcctcttgtcccacagatatccagaaccctctaagaagttcctgctttaaatatttatttatataatcgattaattaatattaatgaatgtggttagagacactagagtgaccctgccgtgtaccagctgagagactctaaatccagtgacaagtctg

SEQ ID NO: 286 - DNA, 179 bp, synthetic construct
  misc_feature 1..179: note = TCGA-184
  source 1..179: mol_type = other DNA
gagatcatgtcctaaccctgatcctcttgtcccacagatatccagaaccctctaagaagttcctgctatttaaatatttatttatataatcgattaattaatattagaatgtggttagagacactagcatgaccctgccgtgtaccagctgagagactctaaatccagtgacaagtctg

SEQ ID NO: 287 - DNA, 179 bp, synthetic construct
  misc_feature 1..179: note = TCGA-185
  source 1..179: mol_type = other DNA
gagatcatgtcctaaccctgatcctcttgtcccacagatatccagaaccctctaagaagttcctgcttaatttaaatatttatttatataatcgattaattaatatgaatgtggttagagacactagtttgaccctgccgtgtaccagctgagagactctaaatccagtgacaagtctg

SEQ ID NO: 288 - DNA, 179 bp, synthetic construct
  misc_feature 1..179: note = TCGA-186
  source 1..179: mol_type = other DNA
gagatcatgtcctaaccctgatcctcttgtcccacagatatccagaaccctctaagaagttcctgctattaatttaaatatttatttatataatcgattaattaatgaatgtggttagagacactatcctgaccctgccgtgtaccagctgagagactctaaatccagtgacaagtctg

SEQ ID NO: 289 - DNA, 179 bp, synthetic construct
  misc_feature 1..179: note = TCGA-187
  source 1..179: mol_type = other DNA
gagatcatgtcctaaccctgatcctcttgtcccacagatatccagaaccctctaagaagttcctgctatattaatttaaatatttatttatataatcgattaattagaatgtggttagagacactatgatgaccctgccgtgtaccagctgagagactctaaatccagtgacaagtctg

SEQ ID NO: 290 - DNA, 179 bp, synthetic construct
  misc_feature 1..179: note = TCGA-188
  source 1..179: mol_type = other DNA
gagatcatgtcctaaccctgatcctcttgtcccacagatatccagaaccctctaagaagttcctgcttaatattaatttaaatatttatttatataatcgattaatgaatgtggttagagacactcaaatgaccctgccgtgtaccagctgagagactctaaatccagtgacaagtctg

SEQ ID NO: 291 - DNA, 179 bp, synthetic construct
  misc_feature 1..179: note = TCGA-189
  source 1..179: mol_type = other DNA
gagatcatgtcctaaccctgatcctcttgtcccacagatatccagaaccctctaagaagttcctgctattaatattaatttaaatatttatttatataatcgattagaatgtggttagagacactcaagtgaccctgccgtgtaccagctgagagactctaaatccagtgacaagtctg

SEQ ID NO: 292 - DNA, 179 bp, synthetic construct
  misc_feature 1..179: note = TCGA-190
  source 1..179: mol_type = other DNA
gagatcatgtcctaaccctgatcctcttgtcccacagatatccagaaccctctaagaagttcctgcttaattaatattaatttaaatatttatttatataatcgatgaatgtggttagagacactctaatgaccctgccgtgtaccagctgagagactctaaatccagtgacaagtctg

SEQ ID NO: 293 - DNA, 179 bp, synthetic construct
  misc_feature 1..179: note = TCGA-191
  source 1..179: mol_type = other DNA
gagatcatgtcctaaccctgatcctcttgtcccacagatatccagaaccctctaagaagttcctgctattaattaatattaatttaaatatttatttatataatcggaatgtggttagagacactctagtgaccctgccgtgtaccagctgagagactctaaatccagtgacaagtctg

SEQ ID NO: 294 - DNA, 150 bp, unidentified
  misc_feature 1..150: note = TC_1
  source 1..150: mol_type = unassigned DNA
gagatcatgtcctaaccctgatcctcttgtcccacagatatccagaaccctctaagaagttcctgctttaatattatatccaagaatgtggttagagacatgaccctgccgtgtaccagctgagagactctaaatccagtgacaagtctg

SEQ ID NO: 295 - DNA, 150 bp, unidentified
  misc_feature 1..150: note = TC_2
  source 1..150: mol_type = unassigned DNA
gagatcatgtcctaaccctgatcctcttgtcccacagatatccagaaccctctaagaagttcctgctattataatatatccatgaatgtggttagagacatgaccctgccgtgtaccagctgagagactctaaatccagtgacaagtctg

SEQ ID NO: 296 - DNA, 150 bp, unidentified
  misc_feature 1..150: note = TC_3
  source 1..150: mol_type = unassigned DNA
gagatcatgtcctaaccctgatcctcttgtcccacagatatccagaaccctctaagaagttcctgctaataataataatccacgaatgtggttagagacatgaccctgccgtgtaccagctgagagactctaaatccagtgacaagtctg

SEQ ID NO: 297 - DNA, 150 bp, unidentified
  misc_feature 1..150: note = TC_4
  source 1..150: mol_type = unassigned DNA
gagatcatgtcctaaccctgatcctcttgtcccacagatatccagaaccctctaagaagttcctgcttaaatatattatccaggaatgtggttagagacatgaccctgccgtgtaccagctgagagactctaaatccagtgacaagtctg

SEQ ID NO: 298 - DNA, 150 bp, unidentified
  misc_feature 1..150: note = TC_5
  source 1..150: mol_type = unassigned DNA
gagatcatgtcctaaccctgatcctcttgtcccacagatatccagaaccctctaagaagttcctgctttaaattataatcctagaatgtggttagagacatgaccctgccgtgtaccagctgagagactctaaatccagtgacaagtctg

SEQ ID NO: 299 - DNA, 150 bp, Una virus
  misc_feature 1..150: note = TC_6
  source 1..150: mol_type = unassigned DNA
gagatcatgtcctaaccctgatcctcttgtcccacagatatccagaaccctctaagaagttcctgctaaaattatttatccttgaatgtggttagagacatgaccctgccgtgtaccagctgagagactctaaatccagtgacaagtctg

SEQ ID NO: 300 - DNA, 150 bp, unidentified
  misc_feature 1..150: note = TC_7
  source 1..150: mol_type = unassigned DNA
gagatcatgtcctaaccctgatcctcttgtcccacagatatccagaaccctctaagaagttcctgcttattattttaatcctcgaatgtggttagagacatgaccctgccgtgtaccagctgagagactctaaatccagtgacaagtctg

SEQ ID NO: 301 - DNA, 150 bp, unidentified
  misc_feature 1..150: note = TC_8
  source 1..150: mol_type = unassigned DNA
gagatcatgtcctaaccctgatcctcttgtcccacagatatccagaaccctctaagaagttcctgcttttataattaatcctggaatgtggttagagacatgaccctgccgtgtaccagctgagagactctaaatccagtgacaagtctg

SEQ ID NO: 302 - DNA, 150 bp, unidentified
  misc_feature 1..150: note = TC_9
  source 1..150: mol_type = unassigned DNA
gagatcatgtcctaaccctgatcctcttgtcccacagatatccagaaccctctaagaagttcctgctaatatattatatcccagaatgtggttagagacatgaccctgccgtgtaccagctgagagactctaaatccagtgacaagtctg

SEQ ID NO: 303 - DNA, 150 bp, unidentified
  misc_feature 1..150: note = TC_10
  source 1..150: mol_type = unassigned DNA
gagatcatgtcctaaccctgatcctcttgtcccacagatatccagaaccctctaagaagttcctgctttaaatatatatccctgaatgtggttagagacatgaccctgccgtgtaccagctgagagactctaaatccagtgacaagtctg

SEQ ID NO: 304 - DNA, 150 bp, unidentified
  misc_feature 1..150: note = TC_11
  source 1..150: mol_type = unassigned DNA
gagatcatgtcctaaccctgatcctcttgtcccacagatatccagaaccctctaagaagttcctgctttaataaataatccccgaatgtggttagagacatgaccctgccgtgtaccagctgagagactctaaatccagtgacaagtctg

SEQ ID NO: 305 - DNA, 150 bp, unidentified
  misc_feature 1..150: note = TC_12
  source 1..150: mol_type = unassigned DNA
gagatcatgtcctaaccctgatcctcttgtcccacagatatccagaaccctctaagaagttcctgcttaaaatttaaatcccggaatgtggttagagacatgaccctgccgtgtaccagctgagagactctaaatccagtgacaagtctg

SEQ ID NO: 306 - DNA, 150 bp, unidentified
  misc_feature 1..150: note = TC_13
  source 1..150: mol_type = unassigned DNA
gagatcatgtcctaaccctgatcctcttgtcccacagatatccagaaccctctaagaagttcctgcttattaaatatatccgagaatgtggttagagacatgaccctgccgtgtaccagctgagagactctaaatccagtgacaagtctg

SEQ ID NO: 307 - DNA, 150 bp, unidentified
  misc_feature 1..150: note = TC_14
  source 1..150: mol_type = unassigned DNA
gagatcatgtcctaaccctgatcctcttgtcccacagatatccagaaccctctaagaagttcctgctaatataaattatccgtgaatgtggttagagacatgaccctgccgtgtaccagctgagagactctaaatccagtgacaagtctg

SEQ ID NO: 308 - DNA, 150 bp, unidentified
  misc_feature 1..150: note = TC_15
  source 1..150: mol_type = unassigned DNA
gagatcatgtcctaaccctgatcctcttgtcccacagatatccagaaccctctaagaagttcctgcttaaaataaaaatccgcgaatgtggttagagacatgaccctgccgtgtaccagctgagagactctaaatccagtgacaagtctg

SEQ ID NO: 309 - DNA, 150 bp, unidentified
  misc_feature 1..150: note = TC_16
  source 1..150: mol_type = unassigned DNA
gagatcatgtcctaaccctgatcctcttgtcccacagatatccagaaccctctaagaagttcctgctataaaattatatccgggaatgtggttagagacatgaccctgccgtgtaccagctgagagactctaaatccagtgacaagtctg

SEQ ID NO: 310 - DNA, 150 bp, unidentified
  misc_feature 1..150: note = TC_17
  source 1..150: mol_type = unassigned DNA
gagatcatgtcctaaccctgatcctcttgtcccacagatatccagaaccctctaagaagttcctgctaaatttttttttccaagaatgtggttagagacatgaccctgccgtgtaccagctgagagactctaaatccagtgacaagtctg

SEQ ID NO: 311 - DNA, 150 bp, unidentified
  misc_feature 1..150: note = TC_18
  source 1..150: mol_type = unassigned DNA
gagatcatgtcctaaccctgatcctcttgtcccacagatatccagaaccctctaagaagttcctgctatattatatattccatgaatgtggttagagacatgaccctgccgtgtaccagctgagagactctaaatccagtgacaagtctg

SEQ ID NO: 312 - DNA, 150 bp, unidentified
  misc_feature 1..150: note = TC_19
  source 1..150: mol_type = unassigned DNA
gagatcatgtcctaaccctgatcctcttgtcccacagatatccagaaccctctaagaagttcctgcttatttaattattccacgaatgtggttagagacatgaccctgccgtgtaccagctgagagactctaaatccagtgacaagtctg

SEQ ID NO: 313 - DNA, 150 bp, unidentified
  misc_feature 1..150: note = TC_20
  source 1..150: mol_type = unassigned DNA
gagatcatgtcctaaccctgatcctcttgtcccacagatatccagaaccctctaagaagttcctgcttttattttaattccaggaatgtggttagagacatgaccctgccgtgtaccagctgagagactctaaatccagtgacaagtctg

SEQ ID NO: 314 - DNA, 150 bp, unidentified
  misc_feature 1..150: note = TC_21
  source 1..150: mol_type = unassigned DNA
gagatcatgtcctaaccctgatcctcttgtcccacagatatccagaaccctctaagaagttcctgcttaatattatattcctagaatgtggttagagacatgaccctgccgtgtaccagctgagagactctaaatccagtgacaagtctg

SEQ ID NO: 315 - DNA, 150 bp, unidentified
  misc_feature 1..150: note = TC_22
  source 1..150: mol_type = unassigned DNA
gagatcatgtcctaaccctgatcctcttgtcccacagatatccagaaccctctaagaagttcctgctataataaaatttccttgaatgtggttagagacatgaccctgccgtgtaccagctgagagactctaaatccagtgacaagtctg

SEQ ID NO: 316 - DNA, 150 bp, unidentified
  misc_feature 1..150: note = TC_23
  source 1..150: mol_type = unassigned DNA
gagatcatgtcctaaccctgatcctcttgtcccacagatatccagaaccctctaagaagttcctgctttttataaaattcctcgaatgtggttagagacatgaccctgccgtgtaccagctgagagactctaaatccagtgacaagtctg

SEQ ID NO: 317 - DNA, 150 bp, unidentified
  misc_feature 1..150: note = TC_24
  source 1..150: mol_type = unassigned DNA
gagatcatgtcctaaccctgatcctcttgtcccacagatatccagaaccctctaagaagttcctgctttatatataattcctggaatgtggttagagacatgaccctgccgtgtaccagctgagagactctaaatccagtgacaagtctg

SEQ ID NO: 318 - DNA, 150 bp, unidentified
  misc_feature 1..150: note = TC_25
  source 1..150: mol_type = unassigned DNA
gagatcatgtcctaaccctgatcctcttgtcccacagatatccagaaccctctaagaagttcctgctaataattttattcccagaatgtggttagagacatgaccctgccgtgtaccagctgagagactctaaatccagtgacaagtctg

SEQ ID NO: 319 - DNA, 150 bp, unidentified
  misc_feature 1..150: note = TC_26
  source 1..150: mol_type = unassigned DNA
gagatcatgtcctaaccctgatcctcttgtcccacagatatccagaaccctctaagaagttcctgctttttaattaattccctgaatgtggttagagacatgaccctgccgtgtaccagctgagagactctaaatccagtgacaagtctg

SEQ ID NO: 320 - DNA, 150 bp, unidentified
  misc_feature 1..150: note = TC_27
  source 1..150: mol_type = unassigned DNA
gagatcatgtcctaaccctgatcctcttgtcccacagatatccagaaccctctaagaagttcctgctatattaatatttccccgaatgtggttagagacatgaccctgccgtgtaccagctgagagactctaaatccagtgacaagtctg

SEQ ID NO: 321 - DNA, 150 bp, unidentified
  misc_feature 1..150: note = TC_28
  source 1..150: mol_type = unassigned DNA
gagatcatgtcctaaccctgatcctcttgtcccacagatatccagaaccctctaagaagttcctgcttaatatatatttcccggaatgtggttagagacatgaccctgccgtgtaccagctgagagactctaaatccagtgacaagtctg

SEQ ID NO: 322 - DNA, 150 bp, unidentified
  misc_feature 1..150: note = TC_29
  source 1..150: mol_type = unassigned DNA
gagatcatgtcctaaccctgatcctcttgtcccacagatatccagaaccctctaagaagttcctgctaataaaatatttccgagaatgtggttagagacatgaccctgccgtgtaccagctgagagactctaaatccagtgacaagtctg

SEQ ID NO: 323 - DNA, 150 bp, unidentified
  misc_feature 1..150: note = TC_30
  source 1..150: mol_type = unassigned DNA
gagatcatgtcctaaccctgatcctcttgtcccacagatatccagaaccctctaagaagttcctgctaatttataatttccgtgaatgtggttagagacatgaccctgccgtgtaccagctgagagactctaaatccagtgacaagtctg

SEQ ID NO: 324 - DNA, 150 bp, unidentified
  misc_feature 1..150: note = TC_31
  source 1..150: mol_type = unassigned DNA
gagatcatgtcctaaccctgatcctcttgtcccacagatatccagaaccctctaagaagttcctgcttaaaattaatttccgcgaatgtggttagagacatgaccctgccgtgtaccagctgagagactctaaatccagtgacaagtctg

SEQ ID NO: 325 - DNA, 150 bp, unidentified
  misc_feature 1..150: note = TC_32
  source 1..150: mol_type = unassigned DNA
gagatcatgtcctaaccctgatcctcttgtcccacagatatccagaaccctctaagaagttcctgctaaattattaattccgggaatgtggttagagacatgaccctgccgtgtaccagctgagagactctaaatccagtgacaagtctg

SEQ ID NO: 326 - DNA, 150 bp, unidentified
  misc_feature 1..150: note = TC_33
  source 1..150: mol_type = unassigned DNA
gagatcatgtcctaaccctgatcctcttgtcccacagatatccagaaccctctaagaagttcctgctattttaaaaactccaagaatgtggttagagacatgaccctgccgtgtaccagctgagagactctaaatccagtgacaagtctg

SEQ ID NO: 327 - DNA, 150 bp, unidentified
  misc_feature 1..150: note = TC_34
  source 1..150: mol_type = unassigned DNA
gagatcatgtcctaaccctgatcctcttgtcccacagatatccagaaccctctaagaagttcctgcttttaaatattctccatgaatgtggttagagacatgaccctgccgtgtaccagctgagagactctaaatccagtgacaagtctg

SEQ ID NO: 328 - DNA, 150 bp, unidentified
  misc_feature 1..150: note = TC_35
  source 1..150: mol_type = unassigned DNA
gagatcatgtcctaaccctgatcctcttgtcccacagatatccagaaccctctaagaagttcctgctattattataactccacgaatgtggttagagacatgaccctgccgtgtaccagctgagagactctaaatccagtgacaagtctg

SEQ ID NO: 329 - DNA, 150 bp, unidentified
  misc_feature 1..150: note = TC_36
  source 1..150: mol_type = unassigned DNA
gagatcatgtcctaaccctgatcctcttgtcccacagatatccagaaccctctaagaagttcctgcttaatttaaatctccaggaatgtggttagagacatgaccctgccgtgtaccagctgagagactctaaatccagtgacaagtctg

SEQ ID NO: 330 - DNA, 150 bp, unidentified
  misc_feature 1..150: note = TC_37
  source 1..150: mol_type = unassigned DNA
gagatcatgtcctaaccctgatcctcttgtcccacagatatccagaaccctctaagaagttcctgcttatataatatctcctagaatgtggttagagacatgaccctgccgtgtaccagctgagagactctaaatccagtgacaagtctg

SEQ ID NO: 331 - DNA, 150 bp, Uahuka affinis
  misc_feature 1..150: note = TC_38
  source 1..150: mol_type = unassigned DNA
gagatcatgtcctaaccctgatcctcttgtcccacagatatccagaaccctctaagaagttcctgctattaaataatctccttgaatgtggttagagacatgaccctgccgtgtaccagctgagagactctaaatccagtgacaagtctg

SEQ ID NO: 332 - DNA, 150 bp, unidentified
  misc_feature 1..150: note = TC_39
  source 1..150: mol_type = unassigned DNA
gagatcatgtcctaaccctgatcctcttgtcccacagatatccagaaccctctaagaagttcctgctattaaatatactcctcgaatgtggttagagacatgaccctgccgtgtaccagctgagagactctaaatccagtgacaagtctg

SEQ ID NO: 333 - DNA, 150 bp, unidentified
  misc_feature 1..150: note = TC_40
  source 1..150: mol_type = unassigned DNA
gagatcatgtcctaaccctgatcctcttgtcccacagatatccagaaccctctaagaagttcctgctaaataattatctcctggaatgtggttagagacatgaccctgccgtgtaccagctgagagactctaaatccagtgacaagtctg

SEQ ID NO: 334 - DNA, 150 bp, unidentified
  misc_feature 1..150: note = TC_41
  source 1..150: mol_type = unassigned DNA
gagatcatgtcctaaccctgatcctcttgtcccacagatatccagaaccctctaagaagttcctgcttaattttttactcccagaatgtggttagagacatgaccctgccgtgtaccagctgagagactctaaatccagtgacaagtctg

SEQ ID NO: 335 - DNA, 150 bp, unidentified
  misc_feature 1..150: note = TC_42
  source 1..150: mol_type = unassigned DNA
gagatcatgtcctaaccctgatcctcttgtcccacagatatccagaaccctctaagaagttcctgctttatattaaactccctgaatgtggttagagacatgaccctgccgtgtaccagctgagagactctaaatccagtgacaagtctg

SEQ ID NO: 336 - DNA, 150 bp, unidentified
  misc_feature 1..150: note = TC_43
  source 1..150: mol_type = unassigned DNA
gagatcatgtcctaaccctgatcctcttgtcccacagatatccagaaccctctaagaagttcctgctttaattaattctccccgaatgtggttagagacatgaccctgccgtgtaccagctgagagactctaaatccagtgacaagtctg

SEQ ID NO: 337 - DNA, 150 bp, unidentified
  misc_feature 1..150: note = TC_44
  source 1..150: mol_type = unassigned DNA
gagatcatgtcctaaccctgatcctcttgtcccacagatatccagaaccctctaagaagttcctgctaattattaaactcccggaatgtggttagagacatgaccctgccgtgtaccagctgagagactctaaatccagtgacaagtctg

SEQ ID NO: 338 - DNA, 150 bp, unidentified
  misc_feature 1..150: note = TC_45
  source 1..150: mol_type = unassigned DNA
gagatcatgtcctaaccctgatcctcttgtcccacagatatccagaaccctctaagaagttcctgctttatttattactccgagaatgtggttagagacatgaccctgccgtgtaccagctgagagactctaaatccagtgacaagtctg

SEQ ID NO: 339 - DNA, 150 bp, unidentified
  misc_feature 1..150: note = TC_46
  source 1..150: mol_type = unassigned DNA
gagatcatgtcctaaccctgatcctcttgtcccacagatatccagaaccctctaagaagttcctgcttttttatatactccgtgaatgtggttagagacatgaccctgccgtgtaccagctgagagactctaaatccagtgacaagtctg

SEQ ID NO: 340 - DNA, 150 bp, unidentified
  misc_feature 1..150: note = TC_47
  source 1..150: mol_type = unassigned DNA
gagatcatgtcctaaccctgatcctcttgtcccacagatatccagaaccctctaagaagttcctgcttttaaattatctccgcgaatgtggttagagacatgaccctgccgtgtaccagctgagagactctaaatccagtgacaagtctg

SEQ ID NO: 341 - DNA, 150 bp, unidentified
  misc_feature 1..150: note = TC_48
  source 1..150: mol_type = unassigned DNA
gagatcatgtcctaaccctgatcctcttgtcccacagatatccagaaccctctaagaagttcctgcttttatataatctccgggaatgtggttagagacatgaccctgccgtgtaccagctgagagactctaaatccagtgacaagtctg

SEQ ID NO: 342 - DNA, 150 bp, unidentified
  misc_feature 1..150: note = TC_49
  source 1..150: mol_type = unassigned DNA
gagatcatgtcctaaccctgatcctcttgtcccacagatatccagaaccctctaagaagttcctgctaaaatattttgtccaagaatgtggttagagacatgaccctgccgtgtaccagctgagagactctaaatccagtgacaagtctg

SEQ ID NO: 343 - DNA, 150 bp, unidentified
  misc_feature 1..150: note = TC_50
  source 1..150: mol_type = unassigned DNA
gagatcatgtcctaaccctgatcctcttgtcccacagatatccagaaccctctaagaagttcctgctatttaaattagtccatgaatgtggttagagacatgaccctgccgtgtaccagctgagagactctaaatccagtgacaagtctg

SEQ ID NO: 344 - DNA, 150 bp, unidentified
  misc_feature 1..150: note = TC_51
  source 1..150: mol_type = unassigned DNA
gagatcatgtcctaaccctgatcctcttgtcccacagatatccagaaccctctaagaagttcctgctattaatatttgtccacgaatgtggttagagacatgaccctgccgtgtaccagctgagagactctaaatccagtgacaagtctg

SEQ ID NO: 345 - DNA, 150 bp, unidentified
  misc_feature 1..150: note = TC_52
  source 1..150: mol_type = unassigned DNA
gagatcatgtcctaaccctgatcctcttgtcccacagatatccagaaccctctaagaagttcctgctataaattttagtccaggaatgtggttagagacatgaccctgccgtgtaccagctgagagactctaaatccagtgacaagtctg

SEQ ID NO: 346 - DNA, 150 bp, unidentified
  misc_feature 1..150: note = TC_53
  source 1..150: mol_type = unassigned DNA
gagatcatgtcctaaccctgatcctcttgtcccacagatatccagaaccctctaagaagttcctgctataaaataaagtcctagaatgtggttagagacatgaccctgccgtgtaccagctgagagactctaaatccagtgacaagtctg

SEQ ID NO: 347 - DNA, 150 bp, unidentified
  misc_feature 1..150: note = TC_54
  source 1..150: mol_type = unassigned DNA
gagatcatgtcctaaccctgatcctcttgtcccacagatatccagaaccctctaagaagttcctgcttatattttatgtccttgaatgtggttagagacatgaccctgccgtgtaccagctgagagactctaaatccagtgacaagtctg

SEQ ID NO: 348 - DNA, 150 bp, unidentified
  misc_feature 1..150: note = TC_55
  source 1..150: mol_type = unassigned DNA
gagatcatgtcctaaccctgatcctcttgtcccacagatatccagaaccctctaagaagttcctgctataattatatgtcctcgaatgtggttagagacatgaccctgccgtgtaccagctgagagactctaaatccagtgacaagtctg

SEQ ID NO: 349 - DNA, 150 bp, unidentified
  misc_feature 1..150: note = TC_56
  source 1..150: mol_type = unassigned DNA
gagatcatgtcctaaccctgatcctcttgtcccacagatatccagaaccctctaagaagttcctgctatttaatattgtcctggaatgtggttagagacatgaccctgccgtgtaccagctgagagactctaaatccagtgacaagtctg

SEQ ID NO: 350 - DNA, 150 bp, unidentified
  misc_feature 1..150: note = TC_57
  source 1..150: mol_type = unassigned DNA
gagatcatgtcctaaccctgatcctcttgtcccacagatatccagaaccctctaagaagttcctgcttttttattatgtcccagaatgtggttagagacatgaccctgccgtgtaccagctgagagactctaaatccagtgacaagtctg

SEQ ID NO: 351 - DNA, 150 bp, unidentified
  misc_feature 1..150: note = TC_58
  source 1..150: mol_type = unassigned DNA
gagatcatgtcctaaccctgatcctcttgtcccacagatatccagaaccctctaagaagttcctgctttaaatttttgtccctgaatgtggttagagacatgaccctgccgtgtaccagctgagagactctaaatccagtgacaagtctg

SEQ ID NO: 352 - DNA, 150 bp, unidentified
  misc_feature 1..150: note = TC_59
  source 1..150: mol_type = unassigned DNA
gagatcatgtcctaaccctgatcctcttgtcccacagatatccagaaccctctaagaagttcctgcttttataaaaagtccccgaatgtggttagagacatgaccctgccgtgtaccagctgagagactctaaatccagtgacaagtctg

SEQ ID NO: 353 - DNA, 150 bp, unidentified
  misc_feature 1..150: note = TC_60
  source 1..150: mol_type = unassigned DNA
gagatcatgtcctaaccctgatcctcttgtcccacagatatccagaaccctctaagaagttcctgctattaaattaagtcccggaatgtggttagagacatgaccctgccgtgtaccagctgagagactctaaatccagtgacaagtctg

SEQ ID NO: 354 - DNA, 150 bp, unidentified
  misc_feature 1..150: note = TC_61
  source 1..150: mol_type = unassigned DNA
gagatcatgtcctaaccctgatcctcttgtcccacagatatccagaaccctctaagaagttcctgctttaataatttgtccgagaatgtggttagagacatgaccctgccgtgtaccagctgagagactctaaatccagtgacaagtctg

SEQ ID NO: 355 - DNA, 150 bp, unidentified
  misc_feature 1..150: note = TC_62
  source 1..150: mol_type = unassigned DNA
gagatcatgtcctaaccctgatcctcttgtcccacagatatccagaaccctctaagaagttcctgctatttttatttgtccgtgaatgtggttagagacatgaccctgccgtgtaccagctgagagactctaaatccagtgacaagtctg

SEQ ID NO: 356 - DNA, 150 bp, unidentified
  misc_feature 1..150: note = TC_63
  source 1..150: mol_type = unassigned DNA
gagatcatgtcctaaccctgatcctcttgtcccacagatatccagaaccctctaagaagttcctgctaaaataattagtccgcgaatgtggttagagacatgaccctgccgtgtaccagctgagagactctaaatccagtgacaagtctg

SEQ ID NO: 357 - DNA, 150 bp, unidentified
  misc_feature 1..150: note = TC_64
  source 1..150: mol_type = unassigned DNA
gagatcatgtcctaaccctgatcctcttgtcccacagatatccagaaccctctaagaagttcctgctttattaataagtccgggaatgtggttagagacatgaccctgccgtgtaccagctgagagactctaaatccagtgacaagtctg

SEQ ID NO: 358 - DNA, 48 bp, Homo sapiens
  misc_feature 1..48: note = CD52 TALE-BE #1 target
  source 1..48: mol_type = genomic DNA
ttttgtcctgagagtccagtttgtatctgtaggaggagaagtgggata

SEQ ID NO: 359 - DNA, 47 bp, Homo sapiens
  misc_feature 1..47: note = CD52 TALE-BE #2 target
  source 1..47: mol_type = genomic DNA
tttgtcctgagagtccagtttgtatctgtaggaggagaagtgggata

SEQ ID NO: 360 - DNA, 46 bp, Homo sapiens
  misc_feature 1..46: note = CD52 TALE-BE #3 target
  source 1..46: mol_type = genomic DNA
ttgtcctgagagtccagtttgtatctgtaggaggagaagtgggata

SEQ ID NO: 361 - DNA, 49 bp, Homo sapiens
  misc_feature 1..49: note = CD52 TALE-BE SP target
  source 1..49: mol_type = genomic DNA
tggctggtgtcgttttgtcctgagagtccagtttgtatctgtaggagga

SEQ ID NO: 362 - DNA, 13 bp, Homo sapiens
  misc_feature 1..13: note = CD52 splice acceptor target site 1
  source 1..13: mol_type = genomic DNA
tacagatacaaac

SEQ ID NO: 363 - DNA, 14 bp, Homo sapiens
  misc_feature 1..14: note = CD52 splice acceptor target site 2
  source 1..14: mol_type = genomic DNA
tacagatacaaact

SEQ ID NO: 364 - DNA, 15 bp, Homo sapiens
  misc_feature 1..15: note = CD52 splice acceptor target site 3
  source 1..15: mol_type = genomic DNA
tacagatacaaactg

SEQ ID NO: 365 - DNA, 15 bp, Homo sapiens
  misc_feature 1..15: note = CD52 signal peptide (SP) editing site
  source 1..15: mol_type = genomic DNA
actggactctcagga

SEQ ID NO: 366 - DNA, 62 bp, Homo sapiens
  misc_feature 1..62: note = TRAC Cluster 0
  source 1..62: mol_type = genomic DNA
atgtcctaaccctgatcctcttgtcccacagatatccagaaccctgaccctgccgtgtacca

SEQ ID NO: 367 - DNA, 62 bp, Homo sapiens
  misc_feature 1..62: note = TRAC Cluster 1
  source 1..62: mol_type = genomic DNA
cctaaccctgatcctcttgtcccacagatatccagaaccctgaccctgccgtgtaccagctg

SEQ ID NO: 368 - DNA, 62 bp, Homo sapiens
  misc_feature 1..62: note = TRAC Cluster 2
  source 1..62: mol_type = genomic DNA
tcctcttgtcccacagatatccagaaccctgaccctgccgtgtaccagctgagagactctaa

SEQ ID NO: 369 - DNA, 62 bp, Homo sapiens
  misc_feature 1..62: note = TRAC Cluster 3
  source 1..62: mol_type = genomic DNA
agaaccctgaccctgccgtgtaccagctgagagactctaaatccagtgacaagtctgtctgc

SEQ ID NO: 370 - DNA, 62 bp, Homo sapiens
  misc_feature 1..62: note = TRAC Cluster 4
  source 1..62: mol_type = genomic DNA
aaccctgaccctgccgtgtaccagctgagagactctaaatccagtgacaagtctgtctgcct

SEQ ID NO: 371 - DNA, 62 bp, Homo sapiens
  misc_feature 1..62: note = TRAC Cluster 5
  source 1..62: mol_type = genomic DNA
cctgaccctgccgtgtaccagctgagagactctaaatccagtgacaagtctgtctgcctatt

SEQ ID NO: 372 - DNA, 62 bp, Homo sapiens
  misc_feature 1..62: note = TRAC Cluster 6
  source 1..62: mol_type = genomic DNA
cctgccgtgtaccagctgagagactctaaatccagtgacaagtctgtctgcctattcaccga

SEQ ID NO: 373 - DNA, 62 bp, Homo sapiens
  misc_feature 1..62: note = TRAC Cluster 7
  source 1..62: mol_type = genomic DNA
gtgtaccagctgagagactctaaatccagtgacaagtctgtctgcctattcaccgattttga

SEQ ID NO: 374 - DNA, 62 bp, Homo sapiens
  misc_feature 1..62: note = TRAC Cluster 8
  source 1..62: mol_type = genomic DNA
cagctgagagactctaaatccagtgacaagtctgtctgcctattcaccgattttgattctca

SEQ ID NO: 375 - DNA, 62 bp, Homo sapiens
  misc_feature 1..62: note = TRAC Cluster 9
  source 1..62: mol_type = genomic DNA
tccagtgacaagtctgtctgcctattcaccgattttgattctcaaacaaatgtgtcacaaag

SEQ ID NO: 376 - DNA, 62 bp, Homo sapiens
  misc_feature 1..62: note = TRAC Cluster 10
  source 1..62: mol_type = genomic DNA
gacaagtctgtctgcctattcaccgattttgattctcaaacaaatgtgtcacaaagtaagga

SEQ ID NO: 377 - DNA, 62 bp, Homo sapiens
  misc_feature 1..62: note = TRAC Cluster 11
  source 1..62: mol_type = genomic DNA
aagtctgtctgcctattcaccgattttgattctcaaacaaatgtgtcacaaagtaaggattc

SEQ ID NO: 378 - DNA, 62 bp, Homo sapiens
  misc_feature 1..62: note = TRAC Cluster 12
  source 1..62: mol_type = genomic DNA
gtctgtctgcctattcaccgattttgattctcaaacaaatgtgtcacaaagtaaggattctg

SEQ ID NO: 379 - DNA, 62 bp, Homo sapiens
  misc_feature 1..62: note = TRAC Cluster 13
  source 1..62: mol_type = genomic DNA
ttcaccgattttgattctcaaacaaatgtgtcacaaagtaaggattctgatgtgtatatcac

SEQ ID NO: 380 - DNA, 62 bp, Homo sapiens
  misc_feature 1..62: note = TRAC Cluster 14
  source 1..62: mol_type = genomic DNA
gattctcaaacaaatgtgtcacaaagtaaggattctgatgtgtatatcacagacaaaactgt

SEQ ID NO: 381 - DNA, 62 bp, Homo sapiens
  misc_feature 1..62: note = TRAC Cluster 15
  source 1..62: mol_type = genomic DNA
tctcaaacaaatgtgtcacaaagtaaggattctgatgtgtatatcacagacaaaactgtgct

SEQ ID NO: 382 - DNA, 62 bp, Homo sapiens
  misc_feature 1..62: note = TRAC Cluster 16
  source 1..62: mol_type = genomic DNA
caaacaaatgtgtcacaaagtaaggattctgatgtgtatatcacagacaaaactgtgctaga

SEQ ID NO: 383 - DNA, 62 bp, Homo sapiens
  misc_feature 1..62: note = TRAC Cluster 17
  source 1..62: mol_type = genomic DNA
caaagtaaggattctgatgtgtatatcacagacaaaactgtgctagacatgaggtctatgga

SEQ ID NO: 384 - DNA, 62 bp, Homo sapiens
  misc_feature 1..62: note = TRAC Cluster 18
  source 1..62: mol_type = genomic DNA
gatgtgtatatcacagacaaaactgtgctagacatgaggtctatggacttcaagagcaacag

SEQ ID NO: 385 - DNA, 62 bp, Homo sapiens
  misc_feature 1..62: note = TRAC Cluster 19
  source 1..62: mol_type = genomic DNA
gtatatcacagacaaaactgtgctagacatgaggtctatggacttcaagagcaacagtgctg

SEQ ID NO: 386 - DNA, 62 bp, Homo sapiens
  misc_feature 1..62: note = TRAC Cluster 20
  source 1..62: mol_type = genomic DNA
atcacagacaaaactgtgctagacatgaggtctatggacttcaagagcaacagtgctgtggc

SEQ ID NO: 387 - DNA, 62 bp, Homo sapiens
  misc_feature 1..62: note = TRAC Cluster 21
  source 1..62: mol_type = genomic DNA
gacaaaactgtgctagacatgaggtctatggacttcaagagcaacagtgctgtggcctggag

SEQ ID NO: 388 - DNA, 62 bp, Homo sapiens
  misc_feature 1..62: note = TRAC Cluster 22
  source 1..62: mol_type = genomic DNA
ggacttcaagagcaacagtgctgtggcctggagcaacaaatctgactttgcatgtgcaaacg

SEQ ID NO: 389 - DNA, 62 bp, Homo sapiens
  misc_feature 1..62: note = TRAC Cluster 23
  source 1..62: mol_type = genomic DNA
agcaacagtgctgtggcctggagcaacaaatctgactttgcatgtgcaaacgccttcaacaa

SEQ ID NO: 390 - DNA, 62 bp, Homo sapiens
  misc_feature 1..62: note = TRAC Cluster 24
  source 1..62: mol_type = genomic DNA
aacagtgctgtggcctggagcaacaaatctgactttgcatgtgcaaacgccttcaacaacag

SEQ ID NO: 391 - DNA, 62 bp, Homo sapiens
  misc_feature 1..62: note = TRAC Cluster 25
  source 1..62: mol_type = genomic DNA
gcaaacgccttcaacaacagcattattccagaagacaccttcttccccagcccaggtaaggg

SEQ ID NO: 392 - DNA, 62 bp, Homo sapiens
  misc_feature 1..62: note = TRAC Cluster 26
  source 1..62: mol_type = genomic DNA
aacgccttcaacaacagcattattccagaagacaccttcttccccagcccaggtaagggcag

SEQ ID NO: 393 - DNA, 62 bp, Homo sapiens
  misc_feature 1..62: note = TRAC Cluster 27
  source 1..62: mol_type = genomic DNA
acaacagcattattccagaagacaccttcttccccagcccaggtaagggcagctttggtgcc

SEQ ID NO: 394 - DNA, 62 bp, Homo sapiens
  misc_feature 1..62: note = TRAC Cluster 28
  source 1..62: mol_type = genomic DNA
atgctgaaagaatgtctgtttttccttttagaaagttcctgtgatgtcaagctggtcgagaa

SEQ ID NO: 395 - DNA, 62 bp, Homo sapiens
  misc_feature 1..62: note = TRAC Cluster 29
  source 1..62: mol_type = genomic DNA
aaagaatgtctgtttttccttttagaaagttcctgtgatgtcaagctggtcgagaaaagctt

SEQ ID NO: 396 - DNA, 62 bp, Homo sapiens
  misc_feature 1..62: note = TRAC Cluster 30
  source 1..62: mol_type = genomic DNA
tgtctgtttttccttttagaaagttcctgtgatgtcaagctggtcgagaaaagctttgaaac

SEQ ID NO: 397 - DNA, 62 bp, Homo sapiens
  misc_feature 1..62: note = TRAC Cluster 31
  source 1..62: mol_type = genomic DNA
ttagaaagttcctgtgatgtcaagctggtcgagaaaagctttgaaacaggtaagacaggggt

SEQ ID NO: 398 - DNA, 62 bp, Homo sapiens
  misc_feature 1..62: note = TRAC Cluster 32
  source 1..62: mol_type = genomic DNA
tgtgatgtcaagctggtcgagaaaagctttgaaacaggtaagacaggggtctagcctgggtt

SEQ ID NO: 399 - DNA, 62 bp, Homo sapiens
  misc_feature 1..62: note = TRAC Cluster 33
  source 1..62: mol_type = genomic DNA
ccataaccgctgtggcctcttggttttacagatacgaacctaaactttcaaaacctgtcagt

SEQ ID NO: 400 - DNA, 62 bp, Homo sapiens
  misc_feature 1..62: note = TRAC Cluster 34
  source 1..62: mol_type = genomic DNA
tcttggttttacagatacgaacctaaactttcaaaacctgtcagtgattgggttccgaatcc

SEQ ID NO: 401 - DNA, 62 bp, Homo sapiens
  misc_feature 1..62: note = TRAC Cluster 35
  source 1..62: mol_type = genomic DNA
acagatacgaacctaaactttcaaaacctgtcagtgattgggttccgaatcctcctcctgaa

SEQ ID NO: 402 - DNA, 62 bp, Homo sapiens
  misc_feature 1..62: note = TRAC Cluster 36
  source 1..62: mol_type = genomic DNA
actttcaaaacctgtcagtgattgggttccgaatcctcctcctgaaagtggccgggtttaat

SEQ ID NO: 403 - DNA, 62 bp, Homo sapiens
  misc_feature 1..62: note = TRAC Cluster 37
  source 1..62: mol_type = genomic DNA
ttcaaaacctgtcagtgattgggttccgaatcctcctcctgaaagtggccgggtttaatctg

SEQ ID NO: 404 - DNA, 62 bp, Homo sapiens
  misc_feature 1..62: note = TRAC Cluster 38
  source 1..62: mol_type = genomic DNA
aaaacctgtcagtgattgggttccgaatcctcctcctgaaagtggccgggtttaatctgctc

SEQ ID NO: 405 - DNA, 62 bp, Homo sapiens
  misc_feature 1..62: note = TRAC Cluster 39
  source 1..62: mol_type = genomic DNA
acctgtcagtgattgggttccgaatcctcctcctgaaagtggccgggtttaatctgctcatg

SEQ ID NO: 406 - DNA, 62 bp, Homo sapiens
  misc_feature 1..62: note = TRAC Cluster 40
  source 1..62: mol_type = genomic DNA
cctgaaagtggccgggtttaatctgctcatgacgctgcggctgtggtccagctgaggtgagg

SEQ ID NO: 407 - DNA, 62 bp, Homo sapiens
  misc_feature 1..62: note = TRAC Cluster 41
  source 1..62: mol_type = genomic DNA
tttaatctgctcatgacgctgcggctgtggtccagctgaggtgaggggccttgaagctggga

SEQ ID NO: 408 - DNA, 62 bp, Homo sapiens
  misc_feature 1..62: note = CD52 Cluster 0
  source 1..62: mol_type = genomic DNA
ccaagacagccacgaagatcctaccaaaatgaagcgcttcctcttcctcctactcaccatca

SEQ ID NO: 409 - DNA, 62 bp, Homo sapiens
  misc_feature 1..62: note = CD52 Cluster 1
  source 1..62: mol_type = genomic DNA
gccacgaagatcctaccaaaatgaagcgcttcctcttcctcctactcaccatcagcctcctg

SEQ ID NO: 410 - DNA, 62 bp, Homo sapiens
  misc_feature 1..62: note = CD52 Cluster 2
  source 1..62: mol_type = genomic DNA
aagatcctaccaaaatgaagcgcttcctcttcctcctactcaccatcagcctcctggttatg

SEQ ID NO: 411 - DNA, 62 bp, Homo sapiens
  misc_feature 1..62: note = CD52 Cluster 3
  source 1..62: mol_type = genomic DNA
atcctaccaaaatgaagcgcttcctcttcctcctactcaccatcagcctcctggttatggta

SEQ ID NO: 412 - DNA, 62 bp, Homo sapiens
  misc_feature 1..62: note = CD52 Cluster 4
  source 1..62: mol_type = genomic DNA
gcttcctcttcctcctactcaccatcagcctcctggttatggtacaggtaagagcaacgcct

SEQ ID NO: 413 - DNA, 62 bp, Homo sapiens
  misc_feature 1..62: note = CD52 Cluster 5
  source 1..62: mol_type = genomic DNA
ccctgatcttatcccacttctcctcctacagatacaaactggactctcaggacaaaacgaca

SEQ ID NO: 414 - DNA, 62 bp, Homo sapiens
  misc_feature 1..62: note = CD52 Cluster 6
  source 1..62: mol_type = genomic DNA
tcccacttctcctcctacagatacaaactggactctcaggacaaaacgacaccagccaaacc

SEQ ID NO: 415 - DNA, 62 bp, Homo sapiens
  misc_feature 1..62: note = CD52 Cluster 7
  source 1..62: mol_type = genomic DNA
cttctcctcctacagatacaaactggactctcaggacaaaacgacaccagccaaaccagcag

SEQ ID NO: 416 - DNA, 62 bp, Homo sapiens
  misc_feature 1..62: note = CD52 Cluster 8
  source 1..62: mol_type = genomic DNA
tcctcctacagatacaaactggactctcaggacaaaacgacaccagccaaaccagcagcccc

SEQ ID NO: 417 - DNA, 62 bp, Homo sapiens
  misc_feature 1..62: note = CD52 Cluster 9
  source 1..62: mol_type = genomic DNA
cagatacaaactggactctcaggacaaaacgacaccagccaaaccagcagcccctcagcatc

SEQ ID NO: 418 - DNA, 62 bp, Homo sapiens
  misc_feature 1..62: note = CD52 Cluster 10
  source 1..62: mol_type = genomic DNA
caaaacgacaccagccaaaccagcagcccctcagcatccagcaacataagcggaggcatttt

SEQ ID NO: 419 - DNA, 62 bp, Homo sapiens
  misc_feature 1..62: note = CD52 Cluster 11
  source 1..62: mol_type = genomic DNA
gacaccagccaaaccagcagcccctcagcatccagcaacataagcggaggcattttcctttt

SEQ ID NO: 420 - DNA, 62 bp, Homo sapiens
  misc_feature 1..62: note = CD52 Cluster 12
  source 1..62: mol_type = genomic DNA
gcagcccctcagcatccagcaacataagcggaggcattttccttttcttcgtggccaatgcc

SEQ ID NO: 421 - DNA, 62 bp, Homo sapiens
  misc_feature 1..62: note = CD52 Cluster 13
  source 1..62: mol_type = genomic DNA
cagcatccagcaacataagcggaggcattttccttttcttcgtggccaatgccataatccac

SEQ ID NO: 422 - DNA, 62 bp, Homo sapiens
  misc_feature 1..62: note = CD52 Cluster 14
  source 1..62: mol_type = genomic DNA
ttttccttttcttcgtggccaatgccataatccacctcttctgcttcagttgaggtgacacg

SEQ ID NO: 423 - DNA, 62 bp, Homo sapiens
  misc_feature 1..62: note = PD1 Cluster 0
  source 1..62: mol_type = genomic DNA
actctggtggggctgctccaggcatgcagatcccacaggcgccctggccagtcgtctgggcg

SEQ ID NO: 424 - DNA, 62 bp, Homo sapiens
  misc_feature 1..62: note = PD1 Cluster 1
  source 1..62: mol_type = genomic DNA
gggcggtgctacaactgggctggcggccaggatggttcttaggtaggtggggtcggcggtca

SEQ ID NO: 425 - DNA, 62 bp, Homo sapiens
  misc_feature 1..62: note = PD1 Cluster 2
  source 1..62: mol_type = genomic DNA
agccccttcctcacctctctccatctctcagactccccagacaggccctggaacccccccac

SEQ ID NO: 426 - DNA, 62 bp, Homo sapiens
  misc_feature 1..62: note = PD1 Cluster 3
  source 1..62: mol_type = genomic DNA
cccttcctcacctctctccatctctcagactccccagacaggccctggaacccccccacctt

SEQ ID NO: 427 - DNA, 62 bp, Homo sapiens
  misc_feature 1..62: note = PD1 Cluster 4
  source 1..62: mol_type = genomic DNA
ctcacctctctccatctctcagactccccagacaggccctggaacccccccaccttctcccc

SEQ ID NO: 428 - DNA, 62 bp, Homo sapiens
  misc_feature 1..62: note = PD1 Cluster 5
  source 1..62: mol_type = genomic DNA
ccagacaggccctggaacccccccaccttctccccagccctgctcgtggtgaccgaagggga

SEQ ID NO: 429 - DNA, 62 bp, Homo sapiens
  misc_feature 1..62: note = PD1 Cluster 6
  source 1..62: mol_type = genomic DNA
accttctccccagccctgctcgtggtgaccgaaggggacaacgccaccttcacctgcagctt

SEQ ID NO: 430 - DNA, 62 bp, Homo sapiens
  misc_feature 1..62: note = PD1 Cluster 7
  source 1..62: mol_type = genomic DNA
tccccagccctgctcgtggtgaccgaaggggacaacgccaccttcacctgcagcttctccaa

SEQ ID NO: 431 - DNA, 62 bp, Homo sapiens
  misc_feature 1..62: note = PD1 Cluster 8
  source 1..62: mol_type = genomic DNA
ggggacaacgccaccttcacctgcagcttctccaacacatcggagagcttcgtgctaaactg

SEQ ID NO: 432 - DNA, 62 bp, Homo sapiens
  misc_feature 1..62: note = PD1 Cluster 9
  source 1..62: mol_type = genomic DNA
gccaccttcacctgcagcttctccaacacatcggagagcttcgtgctaaactggtaccgcat

SEQ ID NO: 433 - DNA, 62 bp, Homo sapiens
  misc_feature 1..62: note = PD1 Cluster 10
  source 1..62: mol_type = genomic DNA
accttcacctgcagcttctccaacacatcggagagcttcgtgctaaactggtaccgcatgag

SEQ ID NO: 434 - DNA, 62 bp, Homo sapiens
  misc_feature 1..62: note = PD1 Cluster 11
  source 1..62: mol_type = genomic DNA
ggagagcttcgtgctaaactggtaccgcatgagccccagcaaccagacggacaagctggccg

SEQ ID NO: 435 - DNA, 62 bp, Homo sapiens
  misc_feature 1..62: note = PD1 Cluster 12
  source 1..62: mol_type = genomic DNA
tggtaccgcatgagccccagcaaccagacggacaagctggccgccttccccgaggaccgcag

SEQ ID NO: 436 - DNA, 62 bp, Homo sapiens
  misc_feature 1..62: note = PD1 Cluster 13
  source 1..62: mol_type = genomic DNA
aaccagacggacaagctggccgccttccccgaggaccgcagccagcccggccaggactgccg

SEQ ID NO: 437 - DNA, 62 bp, Homo sapiens
  misc_feature 1..62: note = PD1 Cluster 14
  source 1..62: mol_type = genomic DNA
cagacggacaagctggccgccttccccgaggaccgcagccagcccggccaggactgccgctt

SEQ ID NO: 438 - DNA, 62 bp, Homo sapiens
  misc_feature 1..62: note = PD1 Cluster 15
  source 1..62: mol_type = genomic DNA
ttccccgaggaccgcagccagcccggccaggactgccgcttccgtgtcacacaactgcccaa

SEQ ID NO: 439 - DNA, 62 bp, Homo sapiens
  misc_feature 1..62: note = PD1 Cluster 16
  source 1..62: mol_type = genomic DNA
ttccgtgtcacacaactgcccaacgggcgtgacttccacatgagcgtggtcagggcccggcg

SEQ ID NO: 440 - DNA, 62 bp, Homo sapiens
  misc_feature 1..62: note = PD1 Cluster 17
  source 1..62: mol_type = genomic DNA
acaactgcccaacgggcgtgacttccacatgagcgtggtcagggcccggcgcaatgacagcg

SEQ ID NO: 441 - DNA, 62 bp, Homo sapiens
  misc_feature 1..62: note = PD1 Cluster 18
  source 1..62: mol_type = genomic DNA
cacatgagcgtggtcagggcccggcgcaatgacagcggcacctacctctgtggggccatctc

SEQ ID NO: 442 - DNA, 62 bp, Homo sapiens
  misc_feature 1..62: note = PD1 Cluster 19
  source 1..62: mol_type = genomic DNA
gacagcggcacctacctctgtggggccatctccctggcccccaaggcgcagatcaaagagag

SEQ ID NO: 443 - DNA, 62 bp, Homo sapiens
  misc_feature 1..62: note = PD1 Cluster 20
  source 1..62: mol_type = genomic DNA
atctccctggcccccaaggcgcagatcaaagagagcctgcgggcagagctcagggtgacagg

SEQ ID NO: 444 - DNA, 62 bp, Homo sapiens
  misc_feature 1..62: note = PD1 Cluster 21
  source 1..62: mol_type = genomic DNA
gtcctaacccctgacctttgtgcccttccagagagaagggcagaagtgcccacagcccaccc

SEQ ID NO: 445 - DNA, 62 bp, Homo sapiens
  misc_feature 1..62: note = PD1 Cluster 22
  source 1..62: mol_type = genomic DNA
taacccctgacctttgtgcccttccagagagaagggcagaagtgcccacagcccaccccagc

SEQ ID NO: 446 - DNA, 62 bp, Homo sapiens
  misc_feature 1..62: note = PD1 Cluster 23
  source 1..62: mol_type = genomic DNA
gacctttgtgcccttccagagagaagggcagaagtgcccacagcccaccccagcccctcacc

SEQ ID NO: 447 - DNA, 62 bp, Homo sapiens
  misc_feature 1..62: note = PD1 Cluster 24
  source 1..62: mol_type = genomic DNA
gcagaagtgcccacagcccaccccagcccctcacccaggccagccggccagttccaaaccct

SEQ ID NO: 448 - DNA, 62 bp, Homo sapiens
  misc_feature 1..62: note = PD1 Cluster 25
  source 1..62: mol_type = genomic DNA
ctgctagtctgggtcctggccgtcatctgctcccgggccgcacgaggtaacgtcatcccagc

SEQ ID NO: 449 - DNA, 62 bp, Homo sapiens
  misc_feature 1..62: note = PD1 Cluster 26
  source 1..62: mol_type = genomic DNA
tcctggccgtcatctgctcccgggccgcacgaggtaacgtcatcccagcccctcggcctgcc

SEQ ID NO: 450 - DNA, 62 bp, Homo sapiens
  misc_feature 1..62: note = PD1 Cluster 43
  source 1..62: mol_type = genomic DNA
cccaagtgtgtttctctgcagggacaataggagccaggcgcaccggccagcccctggtgagt

SEQ ID NO: 451 - DNA, 62 bp, Homo sapiens
  misc_feature 1..62: note = PD1 Cluster 27
  source 1..62: mol_type = genomic DNA
gggctgactccctctccctttctcctcaaagaaggaggacccctcagccgtgcctgtgttct

SEQ ID NO: 452 - DNA, 62 bp, Homo sapiens
  misc_feature 1..62: note = PD1 Cluster 28
  source 1..62: mol_type = genomic DNA
tgactccctctccctttctcctcaaagaaggaggacccctcagccgtgcctgtgttctctgt

SEQ ID NO: 453 - DNA, 62 bp, Homo sapiens
  misc_feature 1..62: note = PD1 Cluster 29
  source 1..62: mol_type = genomic DNA
ctccctttctcctcaaagaaggaggacccctcagccgtgcctgtgttctctgtggactatgg

SEQ ID NO: 454 - DNA, 62 bp, Homo sapiens
  misc_feature 1..62: note = PD1 Cluster 30
  source 1..62: mol_type = genomic DNA
aaggaggacccctcagccgtgcctgtgttctctgtggactatggggagctggatttccagtg

SEQ ID NO: 455 - DNA, 62 bp, Homo sapiens
  misc_feature 1..62: note = PD1 Cluster 31
  source 1..62: mol_type = genomic DNA
gccgtgcctgtgttctctgtggactatggggagctggatttccagtggcgagagaagacccc

SEQ ID NO: 456 - DNA, 62 bp, Homo sapiens
  misc_feature 1..62: note = PD1 Cluster 32
  source 1..62: mol_type = genomic DNA
gactatggggagctggatttccagtggcgagagaagaccccggagccccccgtgccctgtgt

SEQ ID NO: 457 - DNA, 62 bp, Homo sapiens
  misc_feature 1..62: note = PD1 Cluster 33
  source 1..62: mol_type = genomic DNA
ctggatttccagtggcgagagaagaccccggagccccccgtgccctgtgtccctgagcagac

SEQ ID NO: 458 - DNA, 62 bp, Homo sapiens
  misc_feature 1..62: note = PD1 Cluster 34
  source 1..62: mol_type = genomic DNA
accccggagccccccgtgccctgtgtccctgagcagacggagtatgccaccattgtctttcc

SEQ ID NO: 459 - DNA, 62 bp, Homo sapiens
  misc_feature 1..62: note = PD1 Cluster 35
  source 1..62: mol_type = genomic DNA
ccccccgtgccctgtgtccctgagcagacggagtatgccaccattgtctttcctagcggaat

SEQ ID NO: 460 - DNA, 62 bp, Homo sapiens
  misc_feature 1..62: note = PD1 Cluster 36
  source 1..62: mol_type = genomic DNA
accattgtctttcctagcggaatgggcacctcatcccccgcccgcaggggctcagctgacgg

SEQ ID NO: 461 - DNA, 62 bp, Homo sapiens
  misc_feature 1..62: note = PD1 Cluster 37
  source 1..62: mol_type = genomic DNA
attgtctttcctagcggaatgggcacctcatcccccgcccgcaggggctcagctgacggccc

SEQ ID NO: 462 - DNA, 62 bp, Homo sapiens
  misc_feature 1..62: note = PD1 Cluster 38
  source 1..62: mol_type = genomic DNA
atgggcacctcatcccccgcccgcaggggctcagctgacggccctcggagtgcccagccact

SEQ ID NO: 463 - DNA, 62 bp, Homo sapiens
  misc_feature 1..62: note = PD1 Cluster 39
  source 1..62: mol_type = genomic DNA
acctcatcccccgcccgcaggggctcagctgacggccctcggagtgcccagccactgaggcc

SEQ ID NO: 464 - DNA, 62 bp, Homo sapiens
  misc_feature 1..62: note = PD1 Cluster 40
  source 1..62: mol_type = genomic DNA
gccctcggagtgcccagccactgaggcctgaggatggacactgctcttggcccctctgaccg

SEQ ID NO: 465 - DNA, 62 bp, Homo sapiens
  misc_feature 1..62: note = PD1 Cluster 41
  source 1..62: mol_type = genomic DNA
cctcggagtgcccagccactgaggcctgaggatggacactgctcttggcccctctgaccggc

SEQ ID NO: 466 - DNA, 62 bp, Homo sapiens
  misc_feature 1..62: note = PD1 Cluster 42
  source 1..62: mol_type = genomic DNA
cagccactgaggcctgaggatggacactgctcttggcccctctgaccggcttccttggccac

SEQ ID NO: 467 - DNA, 62 bp, Homo sapiens
  misc_feature 1..62: note = B2m Cluster 0
  source 1..62: mol_type = genomic DNA
gctgacagcattcgggccgagatgtctcgctccgtggccttagctgtgctcgcgctactctc

SEQ ID NO: 468 - DNA, 62 bp, Homo sapiens
  misc_feature 1..62: note = B2m Cluster 1
  source 1..62: mol_type = genomic DNA
tccgtggccttagctgtgctcgcgctactctctctttctggcctggaggctatccagcgtga

SEQ ID NO: 469 - DNA, 62 bp, Homo sapiens
  misc_feature 1..62: note = B2m Cluster 2
  source 1..62: mol_type = genomic DNA
cgtggccttagctgtgctcgcgctactctctctttctggcctggaggctatccagcgtgagt

SEQ ID NO: 470 - DNA, 62 bp, Homo sapiens
  misc_feature 1..62: note = B2m Cluster 3
  source 1..62: mol_type = genomic DNA
gccttagctgtgctcgcgctactctctctttctggcctggaggctatccagcgtgagtctct

SEQ ID NO: 471 - DNA, 62 bp, Homo sapiens
  misc_feature 1..62: note = B2m Cluster 4
  source 1..62: mol_type = genomic DNA
gtgctcgcgctactctctctttctggcctggaggctatccagcgtgagtctctcctaccctc

SEQ ID NO: 472 - DNA, 62 bp, Homo sapiens
  misc_feature 1..62: note = B2m Cluster 5
  source 1..62: mol_type = genomic DNA
tgtgtcttttcccgatattcctcaggtactccaaagattcaggtttactcacgtcatccagc

SEQ ID NO: 473 - DNA, 62 bp, Homo sapiens
  misc_feature 1..62: note = B2m Cluster 6
  source 1..62: mol_type = genomic DNA
ttcccgatattcctcaggtactccaaagattcaggtttactcacgtcatccagcagagaatg

SEQ ID NO: 474 - DNA, 62 bp, Homo sapiens
  misc_feature 1..62: note = B2m Cluster 7
  source 1..62: mol_type = genomic DNA
tcctcaggtactccaaagattcaggtttactcacgtcatccagcagagaatggaaagtcaaa

SEQ ID NO: 475 - DNA, 62 bp, Homo sapiens
  misc_feature 1..62: note = B2m Cluster 8
  source 1..62: mol_type = genomic DNA
tactccaaagattcaggtttactcacgtcatccagcagagaatggaaagtcaaatttcctga

SEQ ID NO: 476 - DNA, 62 bp, Homo sapiens
  misc_feature 1..62: note = B2m Cluster 9
  source 1..62: mol_type = genomic DNA
aagattcaggtttactcacgtcatccagcagagaatggaaagtcaaatttcctgaattgcta

SEQ ID NO: 477 - DNA, 62 bp, Homo sapiens
  misc_feature 1..62: note = B2m Cluster 10
  source 1..62: mol_type = genomic DNA
tactcacgtcatccagcagagaatggaaagtcaaatttcctgaattgctatgtgtctgggtt

SEQ ID NO: 478 - DNA, 62 bp, Homo sapiens
  misc_feature 1..62: note = B2m Cluster 11
  source 1..62: mol_type = genomic DNA
ggaaagtcaaatttcctgaattgctatgtgtctgggtttcatccatccgacattgaagttga

SEQ ID NO: 479 - DNA, 62 bp, Homo sapiens
  misc_feature 1..62: note = B2m Cluster 12
  source 1..62: mol_type = genomic DNA
aaatttcctgaattgctatgtgtctgggtttcatccatccgacattgaagttgacttactga

SEQ ID NO: 480 - DNA, 62 bp, Homo sapiens
  misc_feature 1..62: note = B2m Cluster 13
  source 1..62: mol_type = genomic DNA
tttcctgaattgctatgtgtctgggtttcatccatccgacattgaagttgacttactgaaga

SEQ ID NO: 481 - DNA, 62 bp, Homo sapiens
  misc_feature 1..62: note = B2m Cluster 14
  source 1..62: mol_type = genomic DNA
ctgaattgctatgtgtctgggtttcatccatccgacattgaagttgacttactgaagaatgg

SEQ ID NO: 482 - DNA, 62 bp, Homo sapiens
  misc_feature 1..62: note = B2m Cluster 15
  source 1..62: mol_type = genomic DNA
tatgtgtctgggtttcatccatccgacattgaagttgacttactgaagaatggagagagaat

SEQ ID NO: 483 - DNA, 62 bp, Homo sapiens
  misc_feature 1..62: note = B2m Cluster 16
  source 1..62: mol_type = genomic DNA
tctgggtttcatccatccgacattgaagttgacttactgaagaatggagagagaattgaaaa

SEQ ID NO: 484 - DNA, 62 bp, Homo sapiens
  misc_feature 1..62: note = B2m Cluster 17
  source 1..62: mol_type = genomic DNA
gacattgaagttgacttactgaagaatggagagagaattgaaaaagtggagcattcagactt

SEQ ID NO: 485 - DNA, 62 bp, Homo sapiens
  misc_feature 1..62: note = B2m Cluster 18
  source 1..62: mol_type = genomic DNA
ttgaagttgacttactgaagaatggagagagaattgaaaaagtggagcattcagacttgtct

SEQ ID NO: 486 - DNA, 62 bp, Homo sapiens
  misc_feature 1..62: note = B2m Cluster 19
  source 1..62: mol_type = genomic DNA
gttgacttactgaagaatggagagagaattgaaaaagtggagcattcagacttgtctttcag

SEQ ID NO: 487 - DNA, 62 bp, Homo sapiens
  misc_feature 1..62: note = B2m Cluster 20
  source 1..62: mol_type = genomic DNA
ctgaagaatggagagagaattgaaaaagtggagcattcagacttgtctttcagcaaggactg

SEQ ID NO: 488 - DNA, 62 bp, Homo sapiens
  misc_feature 1..62: note = B2m Cluster 21
  source 1..62: mol_type = genomic DNA
aatggagagagaattgaaaaagtggagcattcagacttgtctttcagcaaggactggtcttt

SEQ ID NO: 489 - DNA, 62 bp, Homo sapiens
  misc_feature 1..62: note = B2m Cluster 22
  source 1..62: mol_type = genomic DNA
ggagagagaattgaaaaagtggagcattcagacttgtctttcagcaaggactggtctttcta

SEQ ID NO: 490 - DNA, 62 bp, Homo sapiens
  misc_feature 1..62: note = B2m Cluster 23
  source 1..62: mol_type = genomic DNA
agaattgaaaaagtggagcattcagacttgtctttcagcaaggactggtctttctatctctt

SEQ ID NO: 491 - DNA, 62 bp, Homo sapiens
  misc_feature 1..62: note = B2m Cluster 24
  source 1..62: mol_type = genomic DNA
gtggagcattcagacttgtctttcagcaaggactggtctttctatctcttgtactacactga

SEQ ID NO: 492 - DNA, 62 bp, Homo sapiens
  misc_feature 1..62: note = B2m Cluster 25
  source 1..62: mol_type = genomic DNA
cattcagacttgtctttcagcaaggactggtctttctatctcttgtactacactgaattcac

SEQ ID NO: 493 - DNA, 62 bp, Homo sapiens
  misc_feature 1..62: note = B2m Cluster 26
  source 1..62: mol_type = genomic DNA
cttgtctttcagcaaggactggtctttctatctcttgtactacactgaattcacccccactg

SEQ ID NO: 494 - DNA, 62 bp, Homo sapiens
  misc_feature 1..62: note = B2m Cluster 27
  source 1..62: mol_type = genomic DNA
gactggtctttctatctcttgtactacactgaattcacccccactgaaaaagatgagtatgc

SEQ ID NO: 495 - DNA, 62 bp, Homo sapiens
  misc_feature 1..62: note = B2m Cluster 28
  source 1..62: mol_type = genomic DNA
ctcttgtactacactgaattcacccccactgaaaaagatgagtatgcctgccgtgtgaacca

SEQ ID NO: 496 - DNA, 62 bp, Homo sapiens
  misc_feature 1..62: note = B2m Cluster 29
  source 1..62: mol_type = genomic DNA
tactacactgaattcacccccactgaaaaagatgagtatgcctgccgtgtgaaccatgtgac

SEQ ID NO: 497 - DNA, 62 bp, Homo sapiens
  misc_feature 1..62: note = B2m Cluster 30
  source 1..62: mol_type = genomic DNA
tacactgaattcacccccactgaaaaagatgagtatgcctgccgtgtgaaccatgtgacttt

SEQ ID NO: 498 - DNA, 62 bp, Homo sapiens
  misc_feature 1..62: note = B2m Cluster 31
  source 1..62: mol_type = genomic DNA
tatgcctgccgtgtgaaccatgtgactttgtcacagcccaagatagttaagtggggtaagtc

SEQ ID NO: 499 - DNA, 62 bp, Homo sapiens
  misc_feature 1..62: note = B2m Cluster 32
  source 1..62: mol_type = genomic DNA
cttttttttctccactgtctttttcatagatcgagacatgtaagcagcatcatggaggtaag

SEQ ID NO: 500 - DNA, 62 bp, Homo sapiens
  misc_feature 1..62: note = B2m Cluster 33
  source 1..62: mol_type = genomic DNA
tttttttctccactgtctttttcatagatcgagacatgtaagcagcatcatggaggtaagtt

SEQ ID NO: 501 - DNA, 62 bp, Homo sapiens
  misc_feature 1..62: note = B2m Cluster 34
  source 1..62: mol_type = genomic DNA
tttttctccactgtctttttcatagatcgagacatgtaagcagcatcatggaggtaagtttt

SEQ ID NO: 502 - DNA, 62 bp, Homo sapiens
  misc_feature 1..62: note = ApoC3 Cluster 0
  source 1..62: mol_type = genomic DNA
ggaacagaggtgccatgcagccccgggtactccttgttgttgccctcctggcgctcctggcc

SEQ ID NO: 503 - DNA, 62 bp, Homo sapiens
  misc_feature 1..62: note = ApoC3 Cluster 1
  source 1..62: mol_type = genomic DNA
cttgttgttgccctcctggcgctcctggcctctgcccgtaagcacttggtgggactgggctg

SEQ ID NO: 504 - DNA, 62 bp, Homo sapiens
  misc_feature 1..62: note = ApoC3 Cluster 2
  source 1..62: mol_type = genomic DNA
ccaccccactcagccctgctctttcctcaggagcttcagaggccgaggatgcctcccttctc

SEQ ID NO: 505 - DNA, 62 bp, Homo sapiens
  misc_feature 1..62: note = ApoC3 Cluster 3
  source 1..62: mol_type = genomic DNA
ccactcagccctgctctttcctcaggagcttcagaggccgaggatgcctcccttctcagctt

SEQ ID NO: 506 - DNA, 62 bp, Homo sapiens
  misc_feature 1..62: note = ApoC3 Cluster 4
  source 1..62: mol_type = genomic DNA
ctcagccctgctctttcctcaggagcttcagaggccgaggatgcctcccttctcagcttcat

SEQ ID NO: 507 - DNA, 62 bp, Homo sapiens
  misc_feature 1..62: note = ApoC3 Cluster 5
  source 1..62: mol_type = genomic DNA
tcctcaggagcttcagaggccgaggatgcctcccttctcagcttcatgcagggttacatgaa

SEQ ID NO: 508 - DNA, 62 bp, Homo sapiens
  misc_feature 1..62: note = ApoC3 Cluster 6
  source 1..62: mol_type = genomic DNA
aggagcttcagaggccgaggatgcctcccttctcagcttcatgcagggttacatgaagcacg

SEQ ID NO: 509 - DNA, 62 bp, Homo sapiens
  misc_feature 1..62: note = ApoC3 Cluster 7
  source 1..62: mol_type = genomic DNA
ctcccttctcagcttcatgcagggttacatgaagcacgccaccaagaccgccaaggatgcac

SEQ ID NO: 510 - DNA, 62 bp, Homo sapiens
  misc_feature 1..62: note = ApoC3 Cluster 8
  source 1..62: mol_type = genomic DNA
tacatgaagcacgccaccaagaccgccaaggatgcactgagcagcgtgcaggagtcccaggt

SEQ ID NO: 511 - DNA, 62 bp, Homo sapiens
  misc_feature 1..62: note = ApoC3 Cluster 9
  source 1..62: mol_type = genomic DNA
accgccaaggatgcactgagcagcgtgcaggagtcccaggtggcccagcaggccaggtacac

SEQ ID NO: 512 - DNA, 62 bp, Homo sapiens
  misc_feature 1..62: note = ApoC3 Cluster 10
  source 1..62: mol_type = genomic DNA
gccaaggatgcactgagcagcgtgcaggagtcccaggtggcccagcaggccaggtacacccg

SEQ ID NO: 513 - DNA, 62 bp, Homo sapiens
  misc_feature 1..62: note = ApoC3 Cluster 11
  source 1..62: mol_type = genomic DNA
tttaggggctgggtgaccgatggcttcagttccctgaaagactactggagcaccgttaagga

SEQ ID NO: 514 - DNA, 62 bp, Homo sapiens
  misc_feature 1..62: note = ApoC3 Cluster 12
  source 1..62: mol_type = genomic DNA
tgggtgaccgatggcttcagttccctgaaagactactggagcaccgttaaggacaagttctc

SEQ ID NO: 515 - DNA, 62 bp, Homo sapiens
  misc_feature 1..62: note = ApoC3 Cluster 13
  source 1..62: mol_type = genomic DNA
cgatggcttcagttccctgaaagactactggagcaccgttaaggacaagttctctgagttct

SEQ ID NO: 516 - DNA, 62 bp, Homo sapiens
  misc_feature 1..62: note = ApoC3 Cluster 14
  source 1..62: mol_type = genomic DNA
tccctgaaagactactggagcaccgttaaggacaagttctctgagttctgggatttggaccc

SEQ ID NO: 517 - DNA, 62 bp, Homo sapiens
  misc_feature 1..62: note = ApoC3 Cluster 15
  source 1..62: mol_type = genomic DNA
gactactggagcaccgttaaggacaagttctctgagttctgggatttggaccctgaggtcag

SEQ ID NO: 518 - DNA, 62 bp, Homo sapiens
  misc_feature 1..62: note = ApoC3 Cluster 16
  source 1..62: mol_type = genomic DNA
tactggagcaccgttaaggacaagttctctgagttctgggatttggaccctgaggtcagacc

SEQ ID NO: 519 - DNA, 62 bp, Homo sapiens
  misc_feature 1..62: note = ApoC3 Cluster 17
  source 1..62: mol_type = genomic DNA
accgttaaggacaagttctctgagttctgggatttggaccctgaggtcagaccaacttcagc

SEQ ID NO: 520 - DNA, 62 bp, Homo sapiens
  misc_feature 1..62: note = ApoC3 Cluster 18
  source 1..62: mol_type = genomic DNA
aaggacaagttctctgagttctgggatttggaccctgaggtcagaccaacttcagccgtggc

SEQ ID NO: 521 - DNA, 62 bp, Homo sapiens
  misc_feature 1..62: note = ApoC3 Cluster 19
  source 1..62: mol_type = genomic DNA
aagttctctgagttctgggatttggaccctgaggtcagaccaacttcagccgtggctgcctg

SEQ ID NO: 522 - DNA, 62 bp, Homo sapiens
  misc_feature 1..62: note = ApoC3 Cluster 20
  source 1..62: mol_type = genomic DNA
ctgagttctgggatttggaccctgaggtcagaccaacttcagccgtggctgcctgagacctc

SEQ ID NO: 523 - DNA, 62 bp, Homo sapiens
  misc_feature 1..62: note = ApoC3 Cluster 21
  source 1..62: mol_type = genomic DNA
tgggatttggaccctgaggtcagaccaacttcagccgtggctgcctgagacctcaatacccc

SEQ ID NO: 524 - DNA, 44 bp, Homo sapiens
  misc_feature 1..44: note = Exon1 PK13 splice site 1
  source 1..44: mol_type = genomic DNA
tggaaaagcccggcctgcaccaccagctgtagaaggtgccggga

SEQ ID NO: 525 - DNA, 47 bp, Homo sapiens
  misc_feature 1..47: note = Exon1 PK13 splice site 2
  source 1..47: mol_type = genomic DNA
tggaaaagcccggcctgcaccaccagctgtagaaggtgccgggatga

SEQ ID NO: 526 - DNA, 47 bp, Homo sapiens; identical to SEQ ID NO: 525
  misc_feature 1..47: note = Exon1 PK13 splice site 3
  source 1..47: mol_type = genomic DNA

SEQ ID NO: 527 - DNA, 45 bp, Homo sapiens
  misc_feature 1..45: note = Exon1 PK13 Stop 1
  source 1..45: mol_type = genomic DNA
tgatgtcgaactccagccgctgcttccacacgggctccgagcaca

SEQ ID NO: 528 - DNA, 46 bp, Homo sapiens
  misc_feature 1..46: note = Exon1 PK13 Stop 2
  source 1..46: mol_type = genomic DNA
ttgatgtcgaactccagccgctgcttccacacgggctccgagcaca

SEQ ID NO: 529 - DNA, 48 bp, Homo sapiens
  misc_feature 1..48: note = Exon1 PK13 Stop 3
  source 1..48: mol_type = genomic DNA
tgttgatgtcgaactccagccgctgcttccacacgggctccgagcaca

SEQ ID NO: 530 - DNA, 43 bp, Homo sapiens
  misc_feature 1..43: note = Exon1 PK13 Stop 4
  source 1..43: mol_type = genomic DNA
tgatgtcgaactccagccgctgcttccacacgggctccgagca

SEQ ID NO: 531 - DNA, 44 bp, Homo sapiens
  misc_feature 1..44: note = Exon1 PK13 Stop 5
  source 1..44: mol_type = genomic DNA
tgctcggagcccgtgtggaagcagcggctggagttcgacatcaa

SEQ ID NO: 532 - DNA, 46 bp, Homo sapiens
  misc_feature 1..46: note = Exon1 PK13 Stop 6
  source 1..46: mol_type = genomic DNA
tgttgatgtcgaactccagccgctgcttccacacgggctccgagca

SEQ ID NO: 533 - DNA, 46 bp, Homo sapiens
  misc_feature 1..46: note = Exon2 PK13 splice site 1
  source 1..46: mol_type = genomic DNA
tgaggttggcccaggcaatggggcagtcctgcagaaggacagggca

SEQ ID NO: 534 - DNA, 41 bp, Homo sapiens
  misc_feature 1..41: note = Exon2 PK13 splice site 2
  source 1..41: mol_type = genomic DNA
ttggcccaggcaatggggcagtcctgcagaaggacagggca

SEQ ID NO: 535 - DNA, 46 bp, Homo sapiens
  misc_feature 1..46: note = Exon2 PK13 splice site 3
  source 1..46: mol_type = genomic DNA
ttggcccaggcaatggggcagtcctgcagaaggacagggcaggtga

SEQ ID NO: 536 - DNA, 46 bp, Homo sapiens; identical to SEQ ID NO: 535
  misc_feature 1..46: note = Exon2 PK13 splice site 4
  source 1..46: mol_type = genomic DNA

SEQ ID NO: 537 - DNA, 49 bp, Homo sapiens
  misc_feature 1..49: note = Exon2 PK13 Stop 1
  source 1..49: mol_type = genomic DNA
ttgtagtcaaacagcatgaggttggcccaggcaatggggcagtcctgca

SEQ ID NO: 538 - DNA, 48 bp, Homo sapiens
  misc_feature 1..48: note = Exon2 PK13 Stop 2
  source 1..48: mol_type = genomic DNA
tgtagtcaaacagcatgaggttggcccaggcaatggggcagtcctgca

SEQ ID NO: 539 - DNA, 46 bp, Homo sapiens
  misc_feature 1..46: note = Exon2 PK13 Stop 3
  source 1..46: mol_type = genomic DNA
tagtcaaacagcatgaggttggcccaggcaatggggcagtcctgca

SEQ ID NO: 540 - DNA, 48 bp, Homo sapiens
  misc_feature 1..48: note = Exon3 PK13 splice site 1
  source 1..48: mol_type = genomic DNA
tggggttcagcagctcgcccttctcatctgaacacaggggcagatgaa

SEQ ID NO: 541 - DNA, 42 bp, Homo sapiens
  misc_feature 1..42: note = Exon3 PK13 splice site 2
  source 1..42: mol_type = genomic DNA
tcagcagctcgcccttctcatctgaacacaggggcagatgaa

SEQ ID NO: 542 - DNA, 48 bp, Homo sapiens; identical to SEQ ID NO: 540
  misc_feature 1..48: note = Exon3 PK13 splice site 3
  source 1..48: mol_type = genomic DNA

SEQ ID NO: 543 - DNA, 42 bp, Homo sapiens; identical to SEQ ID NO: 541
  misc_feature 1..42: note = Exon3 PK13 splice site 4
  source 1..42: mol_type = genomic DNA

SEQ ID NO: 544 - DNA, 48 bp, Homo sapiens; identical to SEQ ID NO: 540
  misc_feature 1..48: note = Exon3 PK13 splice site 5
  source 1..48: mol_type = genomic DNA

SEQ ID NO: 545 - DNA, 48 bp, Homo sapiens; identical to SEQ ID NO: 540
  misc_feature 1..48: note = Exon3 PK13 splice site 1 minus
  source 1..48: mol_type = genomic DNA

SEQ ID NO: 546 - DNA, 48 bp, Homo sapiens; identical to SEQ ID NO: 540
  misc_feature 1..48: note = Exon3 PK13 splice site 2 minus
  source 1..48: mol_type = genomic DNA

SEQ ID NO: 547 - DNA, 48 bp, Homo sapiens; identical to SEQ ID NO: 540
  misc_feature 1..48: note = Exon3 PK13 splice site 3 minus
  source 1..48: mol_type = genomic DNA

SEQ ID NO: 548 - DNA, 48 bp, Homo sapiens; identical to SEQ ID NO: 540
  misc_feature 1..48: note = Exon3 PK13 splice site 4 minus
  source 1..48: mol_type = genomic DNA

SEQ ID NO: 549 - DNA, 42 bp, Homo sapiens; identical to SEQ ID NO: 541
  misc_feature 1..42: note = Exon3 PK13 splice site 5 minus
  source 1..42: mol_type = genomic DNA

SEQ ID NO: 550 - DNA, 42 bp, Homo sapiens; identical to SEQ ID NO: 541
  misc_feature 1..42: note = Exon3 PK13 splice site 6 minus
  source 1..42: mol_type = genomic DNA

SEQ ID NO: 551 - protein, 43 aa, synthetic construct
  REGION 1..43: note = C40 TALE C-terminus
SIVAQLSRPDPALAALTNDHLVALACLGGRPALDAVKKGLGGS

SEQ ID NO: 552 - protein, 20 aa, synthetic construct
  REGION 1..20: note = C11 TALE C-terminus
SIVAQLSRPDPSGSGSGGGS

SEQ ID NO: 553 - protein, 917 aa, synthetic construct
  REGION 1..917: note = TALEB STAT3 C40 Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 554 - protein, 839 aa, synthetic construct
  REGION 1..839: note = TALEB STAT3 C40 Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 555 - protein, 877 aa, synthetic construct
  REGION 1..877: note = TALEB STAT3 C0 Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALEGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 556 - protein, 799 aa, synthetic construct
  REGION 1..799: note = TALEB STAT3 C0 Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALEGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 557 - protein, 894 aa, synthetic construct
  REGION 1..894: note = TALEB STAT3 C11 Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 558 - protein, 816 aa, synthetic construct
  REGION 1..816: note = TALEB STAT3 C11 Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPSGSGSGGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 559 - DNA, 47 bp, Homo sapiens
  misc_feature 1..47: note = Target sequence integration B2M
  source 1..47: mol_type = genomic DNA
ttagctgtgctcgcgctactctctctttctggcctggaggctatcca

SEQ ID NO: 560 - DNA, 2283 bp, synthetic construct
  misc_feature 1..2283: note = HLAE AAV sequence
  source 1..2283: mol_type = other DNA
cgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctcttagcggcctcgaagctgttatggctccgcggactttaattttaggtggtggcggatccggtggtggcggttctggtggtggcggctccatccagcgtacgcccaaaattcaagtctacagccgacatcctgcagagaacggcaaatctaatttcctgaactgctatgtatcaggctttcaccctagcgatatagaagtggacctgctgaaaaacggagagaggatagaaaaggtcgaacacagcgacctctccttttccaaggactggagcttttatcttctgtattatactgaatttacacccacggaaaaagatgagtatgcgtgccgagtaaaccacgtcacgctgtcacagcccaaaatagtaaaatgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgtctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggac

SEQ ID NO: 561 - DNA, 59 bp, Homo sapiens; identical to SEQ ID NO: 101
  misc_feature 1..59: note = TRAC TALEN inactivation target sequence
  source 1..59: mol_type = genomic DNA

SEQ ID NO: 562 - protein, 925 aa, synthetic construct; identical to SEQ ID NO: 16
  REGION 1..925: note = TRAC TALEN Left

SEQ ID NO: 563 - protein, 925 aa, synthetic construct; identical to SEQ ID NO: 17
  REGION 1..925: note = TRAC TALEN Right

SEQ ID NO: 564 - protein, 925 aa, synthetic construct; identical to SEQ ID NO: 16
  REGION 1..925: note = B2M TALEN Left

SEQ ID NO: 565 - protein, 925 aa, synthetic construct
  REGION 1..925: note = B2M TALEN Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 566 - protein, 668 aa, synthetic construct
  REGION 1..668: note = TRAC TALEB Left
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGGSAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKM

SEQ ID NO: 567 - protein, 883 aa, synthetic construct
  REGION 1..883: note = TRAC TALEB Right
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 568 - DNA, 145 bp, synthetic construct; identical to SEQ ID NO: 103
  misc_feature 1..145: note = TCGA_1
  source 1..145: mol_type = other DNA

SEQ ID NO: 569 - DNA, 145 bp, synthetic construct; identical to SEQ ID NO: 104
  misc_feature 1..145: note = TCGA_2
  source 1..145: mol_type = other DNA

SEQ ID NO: 570 - DNA, 145 bp, synthetic construct
  misc_feature 1..145: note = TCGA_3
  source 1..145: mol_type = other DNA
gagatcatgtcctaaccctgatcctcttgtcccacagatatccagaaccctctaagaagttcctgctaatcggaatgtggttagagacaaaacgatgaccctgccgtgtaccagctgagagactctaaatccagtgacaagtctg

SEQ ID NO: 571 - DNA, 145 bp, synthetic construct
  misc_feature 1..145: note = TCGA_4
  source 1..145: mol_type = other DNA
gagatcatgtcctaaccctgatcctcttgtcccacagatatccagaaccctctaagaagttcctgctgaatcgaatgtggttagagacaaaacggtgaccctgccgtgtaccagctgagagactctaaatccagtgacaagtctg

SEQ ID NO: 572 - DNA, 147 bp, synthetic construct; identical to SEQ ID NO: 107
  misc_feature 1..147: note = TCGA_5
  source 1..147: mol_type = other DNA

SEQ ID NO: 573 - DNA, 147 bp, synthetic construct; identical to SEQ ID NO: 108
  misc_feature 1..147: note = TCGA_6
  source 1..147: mol_type = other DNA

SEQ ID NO: 574 - DNA, 147 bp, synthetic construct; identical to SEQ ID NO: 109
  misc_feature 1..147: note = TCGA_7
  source 1..147: mol_type = other DNA

SEQ ID NO: 575 - DNA, 149 bp, synthetic construct; identical to SEQ ID NO: 110
  misc_feature 1..149: note = TCGA_8
  source 1..149: mol_type = other DNA

SEQ ID NO: 576 - DNA, 149 bp, synthetic construct; identical to SEQ ID NO: 111
  misc_feature 1..149: note = TCGA_9
  source 1..149: mol_type = other DNA

SEQ ID NO: 577 - DNA, 149 bp, synthetic construct; identical to SEQ ID NO: 112
  misc_feature 1..149: note = TCGA_10
  source 1..149: mol_type = other DNA

SEQ ID NO: 578 - DNA, 149 bp, synthetic construct; identical to SEQ ID NO: 113
  misc_feature 1..149: note = TCGA_11
  source 1..149: mol_type = other DNA

SEQ ID NO: 579 - DNA, 151 bp, synthetic construct; identical to SEQ ID NO: 114
  misc_feature 1..151: note = TCGA_12
  source 1..151: mol_type = other DNA

SEQ ID NO: 580 - DNA, 151 bp, synthetic construct; identical to SEQ ID NO: 115
  misc_feature 1..151: note = TCGA_13
  source 1..151: mol_type = other DNA

SEQ ID NO: 581 - DNA, 151 bp, synthetic construct; identical to SEQ ID NO: 116
  misc_feature 1..151: note = TCGA_14
  source 1..151: mol_type = other DNA

SEQ ID NO: 582 - DNA, 151 bp, synthetic construct; identical to SEQ ID NO: 117
  misc_feature 1..151: note = TCGA_15
  source 1..151: mol_type = other DNA

SEQ ID NO: 583 - DNA, 151 bp, synthetic construct; identical to SEQ ID NO: 118
  misc_feature 1..151: note = TCGA_16
  source 1..151: mol_type = other DNA

SEQ ID NO: 584 - DNA, 153 bp, synthetic construct; identical to SEQ ID NO: 119
  misc_feature 1..153: note = TCGA_17
  source 1..153: mol_type = other DNA

SEQ ID NO: 585 - DNA, 153 bp, synthetic construct; identical to SEQ ID NO: 120
  misc_feature 1..153: note = TCGA_18
  source 1..153: mol_type = other DNA

SEQ ID NO: 586 - DNA, 153 bp, synthetic construct; identical to SEQ ID NO: 121
  misc_feature 1..153: note = TCGA_19
  source 1..153: mol_type = other DNA

SEQ ID NO: 587 - DNA, 153 bp, synthetic construct; identical to SEQ ID NO: 122
  misc_feature 1..153: note = TCGA_20
  source 1..153: mol_type = other DNA

SEQ ID NO: 588 - DNA, 153 bp, synthetic construct; identical to SEQ ID NO: 123
  misc_feature 1..153: note = TCGA_21
  source 1..153: mol_type = other DNA

SEQ ID NO: 589 - DNA, 153 bp, synthetic construct; identical to SEQ ID NO: 124
  misc_feature 1..153: note = TCGA_22
  source 1..153: mol_type = other DNA

SEQ ID NO: 590 - DNA, 155 bp, synthetic construct; identical to SEQ ID NO: 125
  misc_feature 1..155: note = TCGA_23
  source 1..155: mol_type = other DNA

SEQ ID NO: 591 - DNA, 155 bp, synthetic construct; identical to SEQ ID NO: 126
  misc_feature 1..155: note = TCGA_24
  source 1..155: mol_type = other DNA

SEQ ID NO: 592 - DNA, 155 bp, synthetic construct; identical to SEQ ID NO: 127
  misc_feature 1..155: note = TCGA_25
  source 1..155: mol_type = other DNA

SEQ ID NO: 593 - DNA, 155 bp, synthetic construct; identical to SEQ ID NO: 128
  misc_feature 1..155: note = TCGA_26
  source 1..155: mol_type = other DNA

SEQ ID NO: 594 - DNA, 155 bp, synthetic construct; identical to SEQ ID NO: 129
  misc_feature 1..155: note = TCGA_27
  source 1..155: mol_type = other DNA

SEQ ID NO: 595 - DNA, 155 bp, synthetic construct; identical to SEQ ID NO: 130
  misc_feature 1..155: note = TCGA_28
  source 1..155: mol_type = other DNA

SEQ ID NO: 596 - DNA, 155 bp, synthetic construct; identical to SEQ ID NO: 131
  misc_feature 1..155: note = TCGA_29
  source 1..155: mol_type = other DNA

SEQ ID NO: 597 - DNA, 157 bp, synthetic construct; identical to SEQ ID NO: 132
  misc_feature 1..157: note = TCGA_30
  source 1..157: mol_type = other DNA

SEQ ID NO: 598 - DNA, 157 bp, synthetic construct; identical to SEQ ID NO: 133
  misc_feature 1..157: note = TCGA_31
  source 1..157: mol_type = other DNA

SEQ ID NO: 599 - DNA, 157 bp, synthetic construct; identical to SEQ ID NO: 134
  misc_feature 1..157: note = TCGA_32
  source 1..157: mol_type = other DNA

SEQ ID NO: 600 - DNA, 157 bp, synthetic construct; identical to SEQ ID NO: 135
  misc_feature 1..157: note = TCGA_33
  source 1..157: mol_type = other DNA

SEQ ID NO: 601 - DNA, 157 bp, synthetic construct; identical to SEQ ID NO: 136
  misc_feature 1..157: note = TCGA_34
  source 1..157: mol_type = other DNA

SEQ ID NO: 602 - DNA, 157 bp, synthetic construct; identical to SEQ ID NO: 137
  misc_feature 1..157: note = TCGA_35
  source 1..157: mol_type = other DNA

SEQ ID NO: 603 - DNA, 157 bp, synthetic construct; identical to SEQ ID NO: 138
  misc_feature 1..157: note = TCGA_36
  source 1..157: mol_type = other DNA

SEQ ID NO: 604 - DNA, 157 bp, synthetic construct; identical to SEQ ID NO: 139
  misc_feature 1..157: note = TCGA_37
  source 1..157: mol_type = other DNA

SEQ ID NO: 605 - DNA, 149 bp, synthetic construct
  misc_feature 1..149: note = TC15P1_pool1_1
  source 1..149: mol_type = other DNA
gagatcatgtcctaaccctgatcctcttgtcccacagatatccagaaccctctaagaagttcctgcttttaaaataatcaaagaatgtggttagagacatgaccctgccgtgtaccagctgagagactctaaatccagtgacaagtctg

SEQ ID NO: 606 - DNA, 149 bp, synthetic construct
  misc_feature 1..149: note = TC15P1_pool1_2
  source 1..149: mol_type = other DNA
gagatcatgtcctaaccctgatcctcttgtcccacagatatccagaaccctctaagaagttcctgctattttaataatcactgaatgtggttagagacatgaccctgccgtgtaccagctgagagactctaaatccagtgacaagtctg

SEQ ID NO: 607 - DNA, 149 bp, synthetic construct
  misc_feature 1..149: note = TC15P1_pool1_3
  source 1..149: mol_type = other DNA
gagatcatgtcctaaccctgatcctcttgtcccacagatatccagaaccctctaagaagttcctgctataaataaaatcagagaatgtggttagagacatgaccctgccgtgtaccagctgagagactctaaatccagtgacaagtctg

SEQ ID NO: 608 - DNA, 149 bp, synthetic construct
  misc_feature 1..149: note = TC15P1_pool1_4
  source 1..149: mol_type = other DNA
gagatcatgtcctaaccctgatcctcttgtcccacagatatccagaaccctctaagaagttcctgctttatatttaatcatagaatgtggttagagacatgaccctgccgtgtaccagctgagagactctaaatccagtgacaagtctg

SEQ ID NO: 609 - DNA, 149 bp, synthetic construct
  misc_feature 1..149: note = TC15P1_pool1_5
  source 1..149: mol_type = other DNA
gagatcatgtcctaaccctgatcctcttgtcccacagatatccagaaccctctaagaagttcctgctaatatattaatccaagaatgtggttagagacatgaccctgccgtgtaccagctgagagactctaaatccagtgacaagtctg

SEQ ID NO: 610 - DNA, 149 bp, synthetic construct
  misc_feature 1..149: note = TC15P1_pool1_6
  source 1..149: mol_type = other DNA
gagatcatgtcctaaccctgatcctcttgtcccacagatatccagaaccctctaagaagttcctgcttttaaataaatcccagaatgtggttagagacatgaccctgccgtgtaccagctgagagactctaaatccagtgacaagtctg

SEQ ID NO: 611 - DNA, 149 bp, synthetic construct
  misc_feature 1..149: note = TC15P1_pool1_7
  source 1..149: mol_type = other DNA
gagatcatgtcctaaccctgatcctcttgtcccacagatatccagaaccctctaagaagttcctgctttaaaaaaaatccgagaatgtggttagagacatgaccctgccgtgtaccagctgagagactctaaatccagtgacaagtctg

SEQ ID NO: 612 - DNA, 149 bp, synthetic construct
  misc_feature 1..149: note = TC15P1_pool1_8
  source 1..149: mol_type = other DNA
gagatcatgtcctaaccctgatcctcttgtcccacagatatccagaaccctctaagaagttcctgcttattatttaatccttgaatgtggttagagacatgaccctgccgtgtaccagctgagagactctaaatccagtgacaagtctg

SEQ ID NO: 613 - DNA, 149 bp, synthetic construct
  misc_feature 1..149: note = TC15P1_pool1_9
  source 1..149: mol_type = other DNA
gagatcatgtcctaaccctgatcctcttgtcccacagatatccagaaccctctaagaagttcctgctttatttaaaatcgatgaatgtggttagagacatgaccctgccgtgtaccagctgagagactctaaatccagtgacaagtctg

SEQ ID NO: 614 - DNA, 149 bp, synthetic construct
  misc_feature 1..149: note = TC15P1_pool1_10
  source 1..149: mol_type = other DNA
gagatcatgtcctaaccctgatcctcttgtcccacagatatccagaaccctctaagaagttcctgctaaaataataatcgcagaatgtggttagagacatgaccctgccgtgtaccagctgagagactctaaatccagtgacaagtctg

SEQ ID NO: 615 - DNA, 149 bp, synthetic construct
  misc_feature 1..149: note = TC15P1_pool1_11
  source 1..149: mol_type = other DNA
gagatcatgtcctaaccctgatcctcttgtcccacagatatccagaaccctctaagaagttcctgctatattaaaaatcggtgaatgtggttagagacatgaccctgccgtgtaccagctgagagactctaaatccagtgacaagtctg

SEQ ID NO: 616 - DNA, 149 bp, synthetic construct
  misc_feature 1..149: note = TC15P1_pool1_12
  source 1..149: mol_type = other DNA
gagatcatgtcctaaccctgatcctcttgtcccacagatatccagaaccctctaagaagttcctgctattaatataatcgtagaatgtggttagagacatgaccctgccgtgtaccagctgagagactctaaatccagtgacaagtctg

SEQ ID NO: 617 - DNA, 149 bp, synthetic construct
  misc_feature 1..149: note = TC15P1_pool1_13
  source 1..149: mol_type = other DNA
gagatcatgtcctaaccctgatcctcttgtcccacagatatccagaaccctctaagaagttcctgctattatattaatctatgaatgtggttagagacatgaccctgccgtgtaccagctgagagactctaaatccagtgacaagtctg

SEQ ID NO: 618 - DNA, 149 bp, synthetic construct
  misc_feature 1..149: note = TC15P1_pool1_14
  source 1..149: mol_type = other DNA
gagatcatgtcctaaccctgatcctcttgtcccacagatatccagaaccctctaagaagttcctgctatatatttaatctctgaatgtggttagagacatgaccctgccgtgtaccagctgagagactctaaatccagtgacaagtctg

SEQ ID NO: 619 - DNA, 149 bp, synthetic construct
  misc_feature 1..149: note = TC15P1_pool1_15
  source 1..149: mol_type = other DNA
gagatcatgtcctaaccctgatcctcttgtcccacagatatccagaaccctctaagaagttcctgctaaaatataaatctgtgaatgtggttagagacatgaccctgccgtgtaccagctgagagactctaaatccagtgacaagtctg

SEQ ID NO: 620 - DNA, 149 bp, synthetic construct
  misc_feature 1..149: note = TC15P1_pool1_16
  source 1..149: mol_type = other DNA
gagatcatgtcctaaccctgatcctcttgtcccacagatatccagaaccctctaagaagttcctgcttataataaaatctttgaatgtggttagagacatgaccctgccgtgtaccagctgagagactctaaatccagtgacaagtctg

SEQ ID NO: 621 - DNA, 149 bp, synthetic construct
  misc_feature 1..149: note = TC15P1_pool1_17
  source 1..149: mol_type = other DNA
gagatcatgtcctaaccctgatcctcttgtcccacagatatccagaaccctctaagaagttcctgcttaataaatactcaatgaatgtggttagagacatgaccctgccgtgtaccagctgagagactctaaatccagtgacaagtctg

SEQ ID NO: 622 - DNA, 149 bp, synthetic construct
  misc_feature 1..149: note = TC15P1_pool1_18
  source 1..149: mol_type = other DNA
gagatcatgtcctaaccctgatcctcttgtcccacagatatccagaaccctctaagaagttcctgctaataataaactcacagaatgtggttagagacatgaccctgccgtgtaccagctgagagactctaaatccagtgacaagtctg

SEQ ID NO: 623 - DNA, 149 bp, synthetic construct
  misc_feature 1..149: note = TC15P1_pool1_19
  source 1..149: mol_type = other DNA
gagatcatgtcctaaccctgatcctcttgtcccacagatatccagaaccctctaagaagttcctgctataataatactcagtgaatgtggttagagacatgaccctgccgtgtaccagctgagagactctaaatccagtgacaagtctg

SEQ ID NO: 624 - DNA, 149 bp, synthetic construct
  misc_feature 1..149: note = TC15P1_pool1_20
  source 1..149: mol_type = other DNA
gagatcatgtcctaaccctgatcctcttgtcccacagatatccagaaccctctaagaagttcctgctttattaaaactcatagaatgtggttagagacatgaccctgccgtgtaccagctgagagactctaaatccagtgacaagtctg

SEQ ID NO: 625 - DNA, 149 bp, synthetic construct
  misc_feature 1..149: note = TC15P1_pool1_21
  source 1..149: mol_type = other DNA
gagatcatgtcctaaccctgatcctcttgtcccacagatatccagaaccctctaagaagttcctgctaattatttactccaagaatgtggttagagacatgaccctgccgtgtaccagctgagagactctaaatccagtgacaagtctg

SEQ ID NO: 626 - DNA, 149 bp, synthetic construct
  misc_feature 1..149: note = TC15P1_pool1_22
  source 1..149: mol_type = other DNA
gagatcatgtcctaaccctgatcctcttgtcccacagatatccagaaccctctaagaagttcctgctaaataattactcccagaatgtggttagagacatgaccctgccgtgtaccagctgagagactctaaatccagtgacaagtctg

SEQ ID NO: 627 - DNA, 149 bp, synthetic construct
  misc_feature 1..149: note = TC15P1_pool1_23
  source 1..149: mol_type = other DNA
gagatcatgtcctaaccctgatcctcttgtcccacagatatccagaaccctctaagaagttcctgcttatttaatactccgtgaatgtggttagagacatgaccctgccgtgtaccagctgagagactctaaatccagtgacaagtctg

SEQ ID NO: 628 - DNA, 149 bp, synthetic construct
  misc_feature 1..149: note = TC15P1_pool1_24
  source 1..149: mol_type = other DNA
gagatcatgtcctaaccctgatcctcttgtcccacagatatccagaaccctctaagaagttcctgctttaatttaactcctagaatgtggttagagacatgaccctgccgtgtaccagctgagagactctaaatccagtgacaagtctg

SEQ ID NO: 629 - DNA, 149 bp, synthetic construct
  misc_feature 1..149: note = TC15P1_pool1_25
  source 1..149: mol_type = other DNA
gagatcatgtcctaaccctgatcctcttgtcccacagatatccagaaccctctaagaagttcctgctaatttaaaactcgatgaatgtggttagagacatgaccctgccgtgtaccagctgagagactctaaatccagtgacaagtctg

SEQ ID NO: 630 - DNA, 149 bp, synthetic construct
  misc_feature 1..149: note = TC15P1_pool1_26
  source 1..149: mol_type = other DNA
gagatcatgtcctaaccctgatcctcttgtcccacagatatccagaaccctctaagaagttcctgcttatttataactcgctgaatgtggttagagacatgaccctgccgtgtaccagctgagagactctaaatccagtgacaagtctg

SEQ ID NO: 631 - DNA, 149 bp, synthetic construct
  misc_feature 1..149: note = TC15P1_pool1_27
  source 1..149: mol_type = other DNA
gagatcatgtcctaaccctgatcctcttgtcccacagatatccagaaccctctaagaagttcctgcttttaattaactcggtgaatgtggttagagacatgaccctgccgtgtaccagctgagagactctaaatccagtgacaagtctg

SEQ ID NO: 632 - DNA, 149 bp, synthetic construct
  misc_feature 1..149: note = TC15P1_pool1_28
  source 1..149: mol_type = other DNA
gagatcatgtcctaaccctgatcctcttgtcccacagatatccagaaccctctaagaagttcctgctttaatataactcgttgaatgtggttagagacatgaccctgccgtgtaccagctgagagactctaaatccagtgacaagtctg

SEQ ID NO: 633 - DNA, 149 bp, synthetic construct
  misc_feature 1..149: note = TC15P1_pool1_29
  source 1..149: mol_type = other DNA
gagatcatgtcctaaccctgatcctcttgtcccacagatatccagaaccctctaagaagttcctgctaaatttaaactctaagaatgtggttagagacatgaccctgccgtgtaccagctgagagactctaaatccagtgacaagtctg

SEQ ID NO: 634 - DNA, 149 bp, synthetic construct
  misc_feature 1..149: note = TC15P1_pool1_30
  source 1..149: mol_type = other DNA
gagatcatgtcctaaccctgatcctcttgtcccacagatatccagaaccctctaagaagttcctgctataaaaaaactctctgaatgtggttagagacatgaccctgccgtgtaccagctgagagactctaaatccagtgacaagtctg

SEQ ID NO: 635 - DNA, 149 bp, synthetic construct
  misc_feature 1..149: note = TC15P1_pool1_31
  source 1..149: mol_type = other DNA
gagatcatgtcctaaccctgatcctcttgtcccacagatatccagaaccctctaagaagttcctgctttaataatactctgagaatgtggttagagacatgaccctgccgtgtaccagctgagagactctaaatccagtgacaagtctg

SEQ ID NO: 636 - DNA, 149 bp, synthetic construct
  misc_feature 1..149: note = TC15P1_pool1_32
  source 1..149: mol_type = other DNA
gagatcatgtcctaaccctgatcctcttgtcccacagatatccagaaccctctaagaagttcctgctattaataaactctttgaatgtggttagagacatgaccctgccgtgtaccagctgagagactctaaatccagtgacaagtctg

SEQ ID NO: 637 - DNA, 149 bp, synthetic construct
  misc_feature 1..149: note = TC15P1_pool1_33
  source 1..149: mol_type = other DNA
gagatcatgtcctaaccctgatcctcttgtcccacagatatccagaaccctctaagaagttcctgctatattaatagtcaaagaatgtggttagagacatgaccctgccgtgtaccagctgagagactctaaatccagtgacaagtctg

SEQ ID NO: 638 - DNA, 149 bp, synthetic construct
  misc_feature 1..149: note = TC15P1_pool1_34
  source 1..149: mol_type = other DNA
gagatcatgtcctaaccctgatcctcttgtcccacagatatccagaaccctctaagaagttcctgctttataattagtcactgaatgtggttagagacatgaccctgccgtgtaccagctgagagactctaaatccagtgacaagtctg

SEQ ID NO: 639 - DNA, 149 bp, synthetic construct
  misc_feature 1..149: note = TC15P1_pool1_35
  source 1..149: mol_type = other DNA
gagatcatgtcctaaccctgatcctcttgtcccacagatatccagaaccctctaagaagttcctgcttatatattagtcagtgaatgtggttagagacatgaccctgccgtgtaccagctgagagactctaaatccagtgacaagtctg

SEQ ID NO: 640 - DNA, 149 bp, synthetic construct
  misc_feature 1..149: note = TC15P1_pool1_36
  source 1..149: mol_type = other DNA
gagatcatgtcctaaccctgatcctcttgtcccacagatatccagaaccctctaagaagttcctgcttatattttagtcatagaatgtggttagagacatgaccctgccgtgtaccagctgagagactctaaatccagtgacaagtctg

SEQ ID NO: 641 - DNA, 149 bp, synthetic construct
  misc_feature 1..149: note = TC15P1_pool1_37
  source 1..149: mol_type = other DNA
gagatcatgtcctaaccctgatcctcttgtcccacagatatccagaaccctctaagaagttcctgcttaatattaagtccatgaatgtggttagagacatgaccctgccgtgtaccagctgagagactctaaatccagtgacaagtctg

SEQ ID NO: 642 - DNA, 149 bp, synthetic construct
  misc_feature 1..149: note = TC15P1_pool1_38
  source 1..149: mol_type = other DNA
gagatcatgtcctaaccctgatcctcttgtcccacagatatccagaaccctctaagaagttcctgcttaaaatttagtcccagaatgtggttagagacatgaccctgccgtgtaccagctgagagactctaaatccagtgacaagtctg

SEQ ID NO: 643 - DNA, 149 bp, synthetic construct
  misc_feature 1..149: note = TC15P1_pool1_39
  source 1..149: mol_type = other DNA
gagatcatgtcctaaccctgatcctcttgtcccacagatatccagaaccctctaagaagttcctgctatataataagtccgtgaatgtggttagagacatgaccctgccgtgtaccagctgagagactctaaatccagtgacaagtctg

SEQ ID NO: 644 - DNA, 149 bp, synthetic construct
  misc_feature 1..149: note = TC15P1_pool1_40
  source 1..149: mol_type = other DNA
gagatcatgtcctaaccctgatcctcttgtcccacagatatccagaaccctctaagaagttcctgctaattaaaaagtcctagaatgtggttagagacatgaccctgccgtgtaccagctgagagactctaaatccagtgacaagtctg

SEQ ID NO: 645 - DNA, 149 bp, synthetic construct
  misc_feature 1..149: note = TC15P1_pool1_41
  source 1..149: mol_type = other DNA
gagatcatgtcctaaccctgatcctcttgtcccacagatatccagaaccctctaagaagttcctgctaataaaatagtcgatgaatgtggttagagacatgaccctgccgtgtaccagctgagagactctaaatccagtgacaagtctg

SEQ ID NO: 646 - DNA, 149 bp, synthetic construct
  misc_feature 1..149: note = TC15P1_pool1_42
  source 1..149: mol_type = other DNA
gagatcatgtcctaaccctgatcctcttgtcccacagatatccagaaccctctaagaagttcctgctaaaaataaagtcgctgaatgtggttagagacatgaccctgccgtgtaccagctgagagactctaaatccagtgacaagtctg

SEQ ID NO: 647 - DNA, 149 bp, synthetic construct
  misc_feature 1..149: note = TC15P1_pool1_43
  source 1..149: mol_type = other DNA
gagatcatgtcctaaccctgatcctcttgtcccacagatatccagaaccctctaagaagttcctgctatttttaaagtcggtgaatgtggttagagacatgaccctgccgtgtaccagctgagagactctaaatccagtgacaagtctg

SEQ ID NO: 648 - DNA, 149 bp, synthetic construct
  misc_feature 1..149: note = TC15P1_pool1_44
  source 1..149: mol_type = other DNA
gagatcatgtcctaaccctgatcctcttgtcccacagatatccagaaccctctaagaagttcctgcttttttaaaagtcgttgaatgtggttagagacatgaccctgccgtgtaccagctgagagactctaaatccagtgacaagtctg

SEQ ID NO: 649 - DNA, 149 bp, synthetic construct
  misc_feature 1..149: note = TC15P1_pool1_45
  source 1..149: mol_type = other DNA
gagatcatgtcctaaccctgatcctcttgtcccacagatatccagaaccctctaagaagttcctgctataattatagtctaagaatgtggttagagacatgaccctgccgtgtaccagctgagagactctaaatccagtgacaagtctg

SEQ ID NO: 650 - DNA, 149 bp, synthetic construct
  misc_feature 1..149: note = TC15P1_pool1_46
  source 1..149: mol_type = other DNA
gagatcatgtcctaaccctgatcctcttgtcccacagatatccagaaccctctaagaagttcctgctaataaataagtctcagaatgtggttagagacatgaccctgccgtgtaccagctgagagactctaaatccagtgacaagtctg

SEQ ID NO: 651 - DNA, 149 bp, synthetic construct
  misc_feature 1..149: note = TC15P1_pool1_47
  source 1..149: mol_type = other DNA
gagatcatgtcctaaccctgatcctcttgtcccacagatatccagaaccctctaagaagttcctgcttatataatagtctgagaatgtggttagagacatgaccctgccgtgtaccagctgagagactctaaatccagtgacaagtctg

SEQ ID NO: 652 - DNA, 149 bp, synthetic construct
  misc_feature 1..149: note = TC15P1_pool1_48
  source 1..149: mol_type = other DNA
gagatcatgtcctaaccctgatcctcttgtcccacagatatccagaaccctctaagaagttcctgcttaaaaaaaagtctttgaatgtggttagagacatgaccctgccgtgtaccagctgagagactctaaatccagtgacaagtctg

SEQ ID NO: 653 - DNA, 149 bp, synthetic construct
  misc_feature 1..149: note = TC15P1_pool1_49
  source 1..149: mol_type = other DNA
gagatcatgtcctaaccctgatcctcttgtcccacagatatccagaaccctctaagaagttcctgctatttaattattcaaagaatgtggttagagacatgaccctgccgtgtaccagctgagagactctaaatccagtgacaagtctg

SEQ ID NO: 654 - DNA, 149 bp, synthetic construct
  misc_feature 1..149: note = TC15P1_pool1_50
  source 1..149: mol_type = other DNA
gagatcatgtcctaaccctgatcctcttgtcccacagatatccagaaccctctaagaagttcctgctttataataattcacagaatgtggttagagacatgaccctgccgtgtaccagctgagagactctaaatccagtgacaagtctg

SEQ ID NO: 655 - DNA, 149 bp, synthetic construct
  misc_feature 1..149: note = TC15P1_pool1_51
  source 1..149: mol_type = other DNA
gagatcatgtcctaaccctgatcctcttgtcccacagatatccagaaccctctaagaagttcctgcttatatataattcagagaatgtggttagagacatgaccctgccgtgtaccagctgagagactctaaatccagtgacaagtctg

SEQ ID NO: 656 - DNA, 149 bp, synthetic construct
  misc_feature 1..149: note = TC15P1_pool1_52
  source 1..149: mol_type = other DNA
gagatcatgtcctaaccctgatcctcttgtcccacagatatccagaaccctctaagaagttcctgctaaaaaaaaattcatagaatgtggttagagacatgaccctgccgtgtaccagctgagagactctaaatccagtgacaagtctg

SEQ ID NO: 657 - DNA, 149 bp, synthetic construct
  misc_feature 1..149: note = TC15P1_pool1_53
  source 1..149: mol_type = other DNA
gagatcatgtcctaaccctgatcctcttgtcccacagatatccagaaccctctaagaagttcctgcttattttaaattccaagaatgtggttagagacatgaccctgccgtgtaccagctgagagactctaaatccagtgacaagtctg

SEQ ID NO: 658 - DNA, 149 bp, synthetic construct
  misc_feature 1..149: note = TC15P1_pool1_54
  source 1..149: mol_type = other DNA
gagatcatgtcctaaccctgatcctcttgtcccacagatatccagaaccctctaagaagttcctgctaatttttaattcccagaatgtggttagagacatgaccctgccgtgtaccagctgagagactctaaatccagtgacaagtctg

SEQ ID NO: 659 - DNA, 149 bp, synthetic construct
  misc_feature 1..149: note = TC15P1_pool1_55
  source 1..149: mol_type = other DNA
gagatcatgtcctaaccctgatcctcttgtcccacagatatccagaaccctctaagaagttcctgctaaattataattccgagaatgtggttagagacatgaccctgccgtgtaccagctgagagactctaaatccagtgacaagtctg

SEQ ID NO: 660 - DNA, 149 bp, synthetic construct
  misc_feature 1..149: note = TC15P1_pool1_56
  source 1..149: mol_type = other DNA
gagatcatgtcctaaccctgatcctcttgtcccacagatatccagaaccctctaagaagttcctgcttttaaattattccttgaatgtggttagagacatgaccctgccgtgtaccagctgagagactctaaatccagtgacaagtctg

SEQ ID NO: 661 - DNA, 149 bp, synthetic construct
  misc_feature 1..149: note = TC15P1_pool1_57
  source 1..149: mol_type = other DNA
gagatcatgtcctaaccctgatcctcttgtcccacagatatccagaaccctctaagaagttcctgcttttaatttattcgaagaatgtggttagagacatgaccctgccgtgtaccagctgagagactctaaatccagtgacaagtctg

SEQ ID NO: 662 - DNA, 149 bp, synthetic construct
  misc_feature 1..149: note = TC15P1_pool1_58
  source 1..149: mol_type = other DNA
gagatcatgtcctaaccctgatcctcttgtcccacagatatccagaaccctctaagaagttcctgctataattaaattcgctgaatgtggttagagacatgaccctgccgtgtaccagctgagagactctaaatccagtgacaagtctg

SEQ ID NO: 663 - DNA, 149 bp, synthetic construct
  misc_feature 1..149: note = TC15P1_pool1_59
  source 1..149: mol_type = other DNA
gagatcatgtcctaaccctgatcctcttgtcccacagatatccagaaccctctaagaagttcctgctttaaaattattcggagaatgtggttagagacatgaccctgccgtgtaccagctgagagactctaaatccagtgacaagtctg

SEQ ID NO: 664 - DNA, 149 bp, synthetic construct
  misc_feature 1..149: note = TC15P1_pool1_60
  source 1..149: mol_type = other DNA
gagatcatgtcctaaccctgatcctcttgtcccacagatatccagaaccctctaagaagttcctgctttatttttattcgttgaatgtggttagagacatgaccctgccgtgtaccagctgagagactctaaatccagtgacaagtctg

SEQ ID NO: 665 - DNA, 149 bp, synthetic construct
  misc_feature 1..149: note = TC15P1_pool1_61
  source 1..149: mol_type = other DNA
gagatcatgtcctaaccctgatcctcttgtcccacagatatccagaaccctctaagaagttcctgcttaattaaaattctatgaatgtggttagagacatgaccctgccgtgtaccagctgagagactctaaatccagtgacaagtctg

SEQ ID NO: 666 - DNA, 149 bp, synthetic construct
  misc_feature 1..149: note = TC15P1_pool1_62
  source 1..149: mol_type = other DNA
gagatcatgtcctaaccctgatcctcttgtcccacagatatccagaaccctctaagaagttcctgcttataaaaaattctcagaatgtggttagagacatgaccctgccgtgtaccagctgagagactctaaatccagtgacaagtctg

SEQ ID NO: 667 - DNA, 149 bp, synthetic construct
  misc_feature 1..149: note = TC15P1_pool1_63
  source 1..149: mol_type = other DNA
gagatcatgtcctaaccctgatcctcttgtcccacagatatccagaaccctctaagaagttcctgcttatatttaattctgtgaatgtggttagagacatgaccctgccgtgtaccagctgagagactctaaatccagtgacaagtctg

SEQ ID NO: 668 - DNA, 149 bp, synthetic construct
  misc_feature 1..149: note = TC15P1_pool1_64
  source 1..149: mol_type = other DNA
gagatcatgtcctaaccctgatcctcttgtcccacagatatccagaaccctctaagaagttcctgcttaaataatattctttgaatgtggttagagacatgaccctgccgtgtaccagctgagagactctaaatccagtgacaagtctg

SEQ ID NO: 669 - DNA, 149 bp, synthetic construct
  misc_feature 1..149: note = TC15P1_pool1_65
  source 1..149: mol_type = other DNA
gagatcatgtcctaaccctgatcctcttgtcccacagatatccagaaccctctaagaagttcctgctaattttatcatcaaagaatgtggttagagacatgaccctgccgtgtaccagctgagagactctaaatccagtgacaagtctg

SEQ ID NO: 670 - DNA, 149 bp, synthetic construct
  misc_feature 1..149: note = TC15P1_pool1_66
  source 1..149: mol_type = other DNA
gagatcatgtcctaaccctgatcctcttgtcccacagatatccagaaccctctaagaagttcctgctatttattacatcactgaatgtggttagagacatgaccctgccgtgtaccagctgagagactctaaatccagtgacaagtctg

SEQ ID NO: 671 - DNA, 149 bp, synthetic construct
  misc_feature 1..149: note = TC15P1_pool1_67
  source 1..149: mol_type = other DNA
gagatcatgtcctaaccctgatcctcttgtcccacagatatccagaaccctctaagaagttcctgctaattttttcatcagtgaatgtggttagagacatgaccctgccgtgtaccagctgagagactctaaatccagtgacaagtctg

SEQ ID NO: 672 - DNA, 149 bp, synthetic construct
  misc_feature 1..149: note = TC15P1_pool1_68
  source 1..149: mol_type = other DNA
gagatcatgtcctaaccctgatcctcttgtcccacagatatccagaaccctctaagaagttcctgctttttaatacatcattgaatgtggttagagacatgaccctgccgtgtaccagctgagagactctaaatccagtgacaagtctg

SEQ ID NO: 673 - DNA, 149 bp, synthetic construct
  misc_feature 1..149: note = TC15P1_pool1_69
  source 1..149: mol_type = other DNA
gagatcatgtcctaaccctgatcctcttgtcccacagatatccagaaccctctaagaagttcctgctaaattttacatccatgaatgtggttagagacatgaccctgccgtgtaccagctgagagactctaaatccagtgacaagtctg

SEQ ID NO: 674 - DNA, 149 bp, synthetic construct
  misc_feature 1..149: note = TC15P1_pool1_70
  source 1..149: mol_type = other DNA
gagatcatgtcctaaccctgatcctcttgtcccacagatatccagaaccctctaagaagttcctgctttttaaatcatccctgaatgtggttagagacatgaccctgccgtgtaccagctgagagactctaaatccagtgacaagtctg

SEQ ID NO: 675 - DNA, 149 bp, synthetic construct
  misc_feature 1..149: note = TC15P1_pool1_71
  source 1..149: mol_type = other DNA
gagatcatgtcctaaccctgatcctcttgtcccacagatatccagaaccctctaagaagttcctgctatatttttcatccgagaatgtggttagagacatgaccctgccgtgtaccagctgagagactctaaatccagtgacaagtctg

SEQ ID NO: 676 - DNA, 149 bp, synthetic construct
  misc_feature 1..149: note = TC15P1_pool1_72
  source 1..149: mol_type = other DNA
gagatcatgtcctaaccctgatcctcttgtcccacagatatccagaaccctctaagaagttcctgcttataaattcatcctagaatgtggttagagacatgaccctgccgtgtaccagctgagagactctaaatccagtgacaagtctg

SEQ ID NO: 677 - DNA, 149 bp, synthetic construct
  misc_feature 1..149: note = TC15P1_pool1_73
  source 1..149: mol_type = other DNA
gagatcatgtcctaaccctgatcctcttgtcccacagatatccagaaccctctaagaagttcctgctaatattaacatcgatgaatgtggttagagacatgaccctgccgtgtaccagctgagagactctaaatccagtgacaagtctg

SEQ ID NO: 678 - DNA, 149 bp, synthetic construct
  misc_feature 1..149: note = TC15P1_pool1_74
  source 1..149: mol_type = other DNA
gagatcatgtcctaaccctgatcctcttgtcccacagatatccagaaccctctaagaagttcctgctaaatttatcatcgctgaatgtggttagagacatgaccctgccgtgtaccagctgagagactctaaatccagtgacaagtctg

SEQ ID NO: 679 - DNA, 149 bp, synthetic construct
  misc_feature 1..149: note = TC15P1_pool1_75
  source 1..149: mol_type = other DNA
gagatcatgtcctaaccctgatcctcttgtcccacagatatccagaaccctctaagaagttcctgctttataaaacatcggtgaatgtggttagagacatgaccctgccgtgtaccagctgagagactctaaatccagtgacaagtctg

SEQ ID NO: 680 - DNA, 149 bp, synthetic construct
  misc_feature 1..149: note = TC15P1_pool1_76
  source 1..149: mol_type = other DNA
gagatcatgtcctaaccctgatcctcttgtcccacagatatccagaaccctctaagaagttcctgcttaaaaatacatcgtagaatgtggttagagacatgaccctgccgtgtaccagctgagagactctaaatccagtgacaagtctg

SEQ ID NO: 681 - DNA, 149 bp, synthetic construct
  misc_feature 1..149: note = TC15P1_pool1_77
  source 1..149: mol_type = other DNA
gagatcatgtcctaaccctgatcctcttgtcccacagatatccagaaccctctaagaagttcctgctattaatttcatctatgaatgtggttagagacatgaccctgccgtgtaccagctgagagactctaaatccagtgacaagtctg

SEQ ID NO: 682 - DNA, 149 bp, synthetic construct
  misc_feature 1..149: note = TC15P1_pool1_78
  source 1..149: mol_type = other DNA
gagatcatgtcctaaccctgatcctcttgtcccacagatatccagaaccctctaagaagttcctgcttatataaacatctctgaatgtggttagagacatgaccctgccgtgtaccagctgagagactctaaatccagtgacaagtctg

SEQ ID NO: 683 - DNA, 149 bp, synthetic construct
  misc_feature 1..149: note = TC15P1_pool1_79
  source 1..149: mol_type = other DNA
gagatcatgtcctaaccctgatcctcttgtcccacagatatccagaaccctctaagaagttcctgctataaattacatctgtgaatgtggttagagacatgaccctgccgtgtaccagctgagagactctaaatccagtgacaagtctg

SEQ ID NO: 684 - DNA, 149 bp, synthetic construct
  misc_feature 1..149: note = TC15P1_pool1_80
  source 1..149: mol_type = other DNA
gagatcatgtcctaaccctgatcctcttgtcccacagatatccagaaccctctaagaagttcctgcttttattatcatctttgaatgtggttagagacatgaccctgccgtgtaccagctgagagactctaaatccagtgacaagtctg

SEQ ID NO: 685 - DNA, 149 bp, synthetic construct
  misc_feature 1..149: note = TC15P1_pool1_81
  source 1..149: mol_type = other DNA
gagatcatgtcctaaccctgatcctcttgtcccacagatatccagaaccctctaagaagttcctgctataaatttcctcaaagaatgtggttagagacatgaccctgccgtgtaccagctgagagactctaaatccagtgacaagtctg

SEQ ID NO: 686 - DNA, 149 bp, synthetic construct
  misc_feature 1..149: note = TC15P1_pool1_82
  source 1..149: mol_type = other DNA
gagatcatgtcctaaccctgatcctcttgtcccacagatatccagaaccctctaagaagttcctgctattatttacctcactgaatgtggttagagacatgaccctgccgtgtaccagctgagagactctaaatccagtgacaagtctg

SEQ ID NO: 687 - DNA, 149 bp, synthetic construct
  misc_feature 1..149: note = TC15P1_pool1_83
  source 1..149: mol_type = other DNA
gagatcatgtcctaaccctgatcctcttgtcccacagatatccagaaccctctaagaagttcctgctaaataaaacctcagtgaatgtggttagagacatgaccctgccgtgtaccagctgagagactctaaatccagtgacaagtctg

SEQ ID NO: 688 - DNA, 149 bp, synthetic construct
  misc_feature 1..149: note = TC15P1_pool1_84
  source 1..149: mol_type = other DNA
gagatcatgtcctaaccctgatcctcttgtcccacagatatccagaaccctctaagaagttcctgcttaattaatcctcatagaatgtggttagagacatgaccctgccgtgtaccagctgagagactctaaatccagtgacaagtctg

SEQ ID NO: 689 - DNA, 149 bp, synthetic construct
  misc_feature 1..149: note = TC15P1_pool1_85
  source 1..149: mol_type = other DNA
gagatcatgtcctaaccctgatcctcttgtcccacagatatccagaaccctctaagaagttcctgctaattataacctccatgaatgtggttagagacatgaccctgccgtgtaccagctgagagactctaaatccagtgacaagtctg

SEQ ID NO: 690 - DNA, 149 bp, synthetic construct
  misc_feature 1..149: note = TC15P1_pool1_86
  source 1..149: mol_type = other DNA
gagatcatgtcctaaccctgatcctcttgtcccacagatatccagaaccctctaagaagttcctgctatattattcctccctgaatgtggttagagacatgaccctgccgtgtaccagctgagagactctaaatccagtgacaagtctg

SEQ ID NO: 691 - DNA, 149 bp, synthetic construct
  misc_feature 1..149: note = TC15P1_pool1_87
  source 1..149: mol_type = other DNA
gagatcatgtcctaaccctgatcctcttgtcccacagatatccagaaccctctaagaagttcctgctttataaatcctccgagaatgtggttagagacatgaccctgccgtgtaccagctgagagactctaaatccagtgacaagtctg

SEQ ID NO: 692 - DNA, 149 bp, synthetic construct
  misc_feature 1..149: note = TC15P1_pool1_88
  source 1..149: mol_type = other DNA
gagatcatgtcctaaccctgatcctcttgtcccacagatatccagaaccctctaagaagttcctgctttttatatcctcctagaatgtggttagagacatgaccctgccgtgtaccagctgagagactctaaatccagtgacaagtctg

SEQ ID NO: 693 - DNA, 149 bp, synthetic construct
  misc_feature 1..149: note = TC15P1_pool1_89
  source 1..149: mol_type = other DNA
gagatcatgtcctaaccctgatcctcttgtcccacagatatccagaaccctctaagaagttcctgctaaaattttcctcgaagaatgtggttagagacatgaccctgccgtgtaccagctgagagactctaaatccagtgacaagtctg

SEQ ID NO: 694 - DNA, 149 bp, synthetic construct
  misc_feature 1..149: note = TC15P1_pool1_90
  source 1..149: mol_type = other DNA
gagatcatgtcctaaccctgatcctcttgtcccacagatatccagaaccctctaagaagttcctgcttattaaaacctcgctgaatgtggttagagacatgaccctgccgtgtaccagctgagagactctaaatccagtgacaagtctg

SEQ ID NO: 695 - DNA, 149 bp, synthetic construct
  misc_feature 1..149: note = TC15P1_pool1_91
  source 1..149: mol_type = other DNA
gagatcatgtcctaaccctgatcctcttgtcccacagatatccagaaccctctaagaagttcctgcttaaattaacctcggagaatgtggttagagacatgaccctgccgtgtaccagctgagagactctaaatccagtgacaagtctg

SEQ ID NO: 696 - DNA, 149 bp, synthetic construct
  misc_feature 1..149: note = TC15P1_pool1_92
  source 1..149: mol_type = other DNA
gagatcatgtcctaaccctgatcctcttgtcccacagatatccagaaccctctaagaagttcctgcttaaatattcctcgtagaatgtggttagagacatgaccctgccgtgtaccagctgagagactctaaatccagtgacaagtctg

SEQ ID NO: 697 - DNA, 149 bp, synthetic construct
  misc_feature 1..149: note = TC15P1_pool1_93
  source 1..149: mol_type = other DNA
gagatcatgtcctaaccctgatcctcttgtcccacagatatccagaaccctctaagaagttcctgctaataattacctctatgaatgtggttagagacatgaccctgccgtgtaccagctgagagactctaaatccagtgacaagtctg

SEQ ID NO: 698 - DNA, 149 bp, synthetic construct
  misc_feature 1..149: note = TC15P1_pool1_94
  source 1..149: mol_type = other DNA
gagatcatgtcctaaccctgatcctcttgtcccacagatatccagaaccctctaagaagttcctgctatataaatcctctctgaatgtggttagagacatgaccctgccgtgtaccagctgagagactctaaatccagtgacaagtctg

SEQ ID NO: 699 - DNA, 149 bp, synthetic construct
  misc_feature 1..149: note = TC15P1_pool1_95
  source 1..149: mol_type = other DNA
gagatcatgtcctaaccctgatcctcttgtcccacagatatccagaaccctctaagaagttcctgctttaaatatcctctgtgaatgtggttagagacatgaccctgccgtgtaccagctgagagactctaaatccagtgacaagtctg

SEQ ID NO: 700 - DNA, 149 bp, synthetic construct
  misc_feature 1..149: note = TC15P1_pool1_96
  source 1..149: mol_type = other DNA
gagatcatgtcctaaccctgatcctcttgtcccacagatatccagaaccctctaagaagttcctgctaaaaatatcctcttagaatgtggttagagacatgaccctgccgtgtaccagctgagagactctaaatccagtgacaagtctg

SEQ ID NO: 701 - DNA, 149 bp, synthetic construct
  misc_feature 1..149: note = TC15P2_pool1_97
  source 1..149: mol_type = other DNA
gagatcatgtcctaaccctgatcctcttgtcccacagatatccagaaccctctaagaagttcctgctatatatatcgtcaaagaatgtggttagagacatgaccctgccgtgtaccagctgagagactctaaatccagtgacaagtctg

SEQ ID NO: 702 - DNA, 149 bp, synthetic construct
  misc_feature 1..149: note = TC15P2_pool1_98
  source 1..149: mol_type = other DNA
gagatcatgtcctaaccctgatcctcttgtcccacagatatccagaaccctctaagaagttcctgctttattaatcgtcactgaatgtggttagagacatgaccctgccgtgtaccagctgagagactctaaatccagtgacaagtctg

SEQ ID NO: 703 - DNA, 149 bp, synthetic construct
  misc_feature 1..149: note = TC15P2_pool1_99
  source 1..149: mol_type = other DNA
gagatcatgtcctaaccctgatcctcttgtcccacagatatccagaaccctctaagaagttcctgctattataaacgtcagagaatgtggttagagacatgaccctgccgtgtaccagctgagagactctaaatccagtgacaagtctg

SEQ ID NO: 704 - DNA, 149 bp, synthetic construct
  misc_feature 1..149: note = TC15P2_pool1_100
  source 1..149: mol_type = other DNA
gagatcatgtcctaaccctgatcctcttgtcccacagatatccagaaccctctaagaagttcctgcttttaaaaacgtcattgaatgtggttagagacatgaccctgccgtgtaccagctgagagactctaaatccagtgacaagtctg

SEQ ID NO: 705 - DNA, 149 bp, synthetic construct
  misc_feature 1..149: note = TC15P2_pool1_101
  source 1..149: mol_type = other DNA
gagatcatgtcctaaccctgatcctcttgtcccacagatatccagaaccctctaagaagttcctgcttataaatacgtccatgaatgtggttagagacatgaccctgccgtgtaccagctgagagactctaaatccagtgacaagtctg

SEQ ID NO: 706 - DNA, 149 bp, synthetic construct
  misc_feature 1..149: note = TC15P2_pool1_102
  source 1..149: mol_type = other DNA
gagatcatgtcctaaccctgatcctcttgtcccacagatatccagaaccctctaagaagttcctgcttttaataacgtcccagaatgtggttagagacatgaccctgccgtgtaccagctgagagactctaaatccagtgacaagtctg

SEQ ID NO: 707 - DNA, 149 bp, synthetic construct
  misc_feature 1..149: note = TC15P2_pool1_103
  source 1..149: mol_type = other DNA
gagatcatgtcctaaccctgatcctcttgtcccacagatatccagaaccctctaagaagttcctgctataattttcgtccgtgaatgtggttagagacatgaccctgccgtgtaccagctgagagactctaaatccagtgacaagtctg

SEQ ID NO: 708 - DNA, 149 bp, synthetic construct
  misc_feature 1..149: note = TC15P2_pool1_104
  source 1..149: mol_type = other DNA
gagatcatgtcctaaccctgatcctcttgtcccacagatatccagaaccctctaagaagttcctgcttttttattcgtcctagaatgtggttagagacatgaccctgccgtgtaccagctgagagactctaaatccagtgacaagtctg

SEQ ID NO: 709 - DNA, 149 bp, synthetic construct
  misc_feature 1..149: note = TC15P2_pool1_105
  source 1..149: mol_type = other DNA
gagatcatgtcctaaccctgatcctcttgtcccacagatatccagaaccctctaagaagttcctgcttaattattcgtcgatgaatgtggttagagacatgaccctgccgtgtaccagctgagagactctaaatccagtgacaagtctg

SEQ ID NO: 710 - DNA, 149 bp, synthetic construct
  misc_feature 1..149: note = TC15P2_pool1_106
  source 1..149: mol_type = other DNA
gagatcatgtcctaaccctgatcctcttgtcccacagatatccagaaccctctaagaagttcctgctataaaaatcgtcgcagaatgtggttagagacatgaccctgccgtgtaccagctgagagactctaaatccagtgacaagtctg

SEQ ID NO: 711 - DNA, 149 bp, synthetic construct
  misc_feature 1..149: note = TC15P2_pool1_107
  source 1..149: mol_type = other DNA
gagatcatgtcctaaccctgatcctcttgtcccacagatatccagaaccctctaagaagttcctgcttataatatcgtcggagaatgtggttagagacatgaccctgccgtgtaccagctgagagactctaaatccagtgacaagtctg

SEQ ID NO: 712 - DNA, 149 bp, synthetic construct
  misc_feature 1..149: note = TC15P2_pool1_108
  source 1..149: mol_type = other DNA
gagatcatgtcctaaccctgatcctcttgtcccacagatatccagaaccctctaagaagttcctgcttaatatatcgtcgtagaatgtggttagagacatgaccctgccgtgtaccagctgagagactctaaatccagtgacaagtctg

SEQ ID NO: 713 - DNA, 149 bp, synthetic construct
  misc_feature 1..149: note = TC15P2_pool1_109
  source 1..149: mol_type = other DNA
gagatcatgtcctaaccctgatcctcttgtcccacagatatccagaaccctctaagaagttcctgcttaatttttcgtctaagaatgtggttagagacatgaccctgccgtgtaccagctgagagactctaaatccagtgacaagtctg

SEQ ID NO: 714 - DNA, 149 bp, synthetic construct
  misc_feature 1..149: note = TC15P2_pool1_110
  source 1..149: mol_type = other DNA
gagatcatgtcctaaccctgatcctcttgtcccacagatatccagaaccctctaagaagttcctgctatttaaaacgtctctgaatgtggttagagacatgaccctgccgtgtaccagctgagagactctaaatccagtgacaagtctg

SEQ ID NO: 715 - DNA, 149 bp, synthetic construct
  misc_feature 1..149: note = TC15P2_pool1_111
  source 1..149: mol_type = other DNA
gagatcatgtcctaaccctgatcctcttgtcccacagatatccagaaccctctaagaagttcctgctatataaaacgtctgagaatgtggttagagacatgaccctgccgtgtaccagctgagagactctaaatccagtgacaagtctg

SEQ ID NO: 716 - DNA, 149 bp, synthetic construct
  misc_feature 1..149: note = TC15P2_pool1_112
  source 1..149: mol_type = other DNA
gagatcatgtcctaaccctgatcctcttgtcccacagatatccagaaccctctaagaagttcctgctataatattcgtcttagaatgtggttagagacatgaccctgccgtgtaccagctgagagactctaaatccagtgacaagtctg

SEQ ID NO: 717 - DNA, 149 bp, synthetic construct
  misc_feature 1..149: note = TC15P2_pool1_113
  source 1..149: mol_type = other DNA
gagatcatgtcctaaccctgatcctcttgtcccacagatatccagaaccctctaagaagttcctgctattattttcttcaaagaatgtggttagagacatgaccctgccgtgtaccagctgagagactctaaatccagtgacaagtctg

SEQ ID NO: 718 - DNA, 149 bp, synthetic construct
  misc_feature 1..149: note = TC15P2_pool1_114
  source 1..149: mol_type = other DNA
gagatcatgtcctaaccctgatcctcttgtcccacagatatccagaaccctctaagaagttcctgctaattaatacttcactgaatgtggttagagacatgaccctgccgtgtaccagctgagagactctaaatccagtgacaagtctg

SEQ ID NO: 719 - DNA, 149 bp, synthetic construct
  misc_feature 1..149: note = TC15P2_pool1_115
  source 1..149: mol_type = other DNA
gagatcatgtcctaaccctgatcctcttgtcccacagatatccagaaccctctaagaagttcctgcttattattacttcagagaatgtggttagagacatgaccctgccgtgtaccagctgagagactctaaatccagtgacaagtctg

SEQ ID NO: 720 - DNA, 149 bp, synthetic construct
  misc_feature 1..149: note = TC15P2_pool1_116
  source 1..149: mol_type = other DNA
gagatcatgtcctaaccctgatcctcttgtcccacagatatccagaaccctctaagaagttcctgctattaaatacttcattgaatgtggttagagacatgaccctgccgtgtaccagctgagagactctaaatccagtgacaagtctg

SEQ ID NO: 721 - DNA, 149 bp, synthetic construct
  misc_feature 1..149: note = TC15P2_pool1_117
  source 1..149: mol_type = other DNA
gagatcatgtcctaaccctgatcctcttgtcccacagatatccagaaccctctaagaagttcctgctaaaaaattcttccatgaatgtggttagagacatgaccctgccgtgtaccagctgagagactctaaatccagtgacaagtctg

SEQ ID NO: 722 - DNA, 149 bp, synthetic construct
  misc_feature 1..149: note = TC15P2_pool1_118
  source 1..149: mol_type = other DNA
gagatcatgtcctaaccctgatcctcttgtcccacagatatccagaaccctctaagaagttcctgctttttaaaacttcccagaatgtggttagagacatgaccctgccgtgtaccagctgagagactctaaatccagtgacaagtctg

SEQ ID NO: 723 - DNA, 149 bp, synthetic construct
  misc_feature 1..149: note = TC15P2_pool1_119
  source 1..149: mol_type = other DNA
gagatcatgtcctaaccctgatcctcttgtcccacagatatccagaaccctctaagaagttcctgcttaaattttcttccgtgaatgtggttagagacatgaccctgccgtgtaccagctgagagactctaaatccagtgacaagtctg

SEQ ID NO: 724 - DNA, 149 bp, synthetic construct
  misc_feature 1..149: note = TC15P2_pool1_120
  source 1..149: mol_type = other DNA
gagatcatgtcctaaccctgatcctcttgtcccacagatatccagaaccctctaagaagttcctgctttatttatcttcctagaatgtggttagagacatgaccctgccgtgtaccagctgagagactctaaatccagtgacaagtctg

SEQ ID NO: 725 - DNA, 149 bp, synthetic construct
  misc_feature 1..149: note = TC15P2_pool1_121
  source 1..149: mol_type = other DNA
gagatcatgtcctaaccctgatcctcttgtcccacagatatccagaaccctctaagaagttcctgctataaaatacttcgaagaatgtggttagagacatgaccctgccgtgtaccagctgagagactctaaatccagtgacaagtctg

SEQ ID NO: 726 - DNA, 149 bp, synthetic construct
  misc_feature 1..149: note = TC15P2_pool1_122
  source 1..149: mol_type = other DNA
gagatcatgtcctaaccctgatcctcttgtcccacagatatccagaaccctctaagaagttcctgcttttttttacttcgcagaatgtggttagagacatgaccctgccgtgtaccagctgagagactctaaatccagtgacaagtctg

SEQ ID NO: 727 - DNA, 149 bp, synthetic construct
  misc_feature 1..149: note = TC15P2_pool1_123
  source 1..149: mol_type = other DNA
gagatcatgtcctaaccctgatcctcttgtcccacagatatccagaaccctctaagaagttcctgcttattaaatcttcggagaatgtggttagagacatgaccctgccgtgtaccagctgagagactctaaatccagtgacaagtctg

SEQ ID NO: 728 - DNA, 149 bp, synthetic construct
  misc_feature 1..149: note = TC15P2_pool1_124
  source 1..149: mol_type = other DNA
gagatcatgtcctaaccctgatcctcttgtcccacagatatccagaaccctctaagaagttcctgctattaattacttcgtagaatgtggttagagacatgaccctgccgtgtaccagctgagagactctaaatccagtgacaagtctg

SEQ ID NO: 729 - DNA, 149 bp, synthetic construct
  misc_feature 1..149: note = TC15P2_pool1_125
  source 1..149: mol_type = other DNA
gagatcatgtcctaaccctgatcctcttgtcccacagatatccagaaccctctaagaagttcctgctaaatattacttctaagaatgtggttagagacatgaccctgccgtgtaccagctgagagactctaaatccagtgacaagtctg

SEQ ID NO: 730 - DNA, 149 bp, synthetic construct
  misc_feature 1..149: note = TC15P2_pool2_1
  source 1..149: mol_type = other DNA
gagatcatgtcctaaccctgatcctcttgtcccacagatatccagaaccctctaagaagttcctgcttttaaaatcttctcagaatgtggttagagacatgaccctgccgtgtaccagctgagagactctaaatccagtgacaagtctg

SEQ ID NO: 731 - DNA, 149 bp, synthetic construct
  misc_feature 1..149: note = TC15P2_pool2_2
  source 1..149: mol_type = other DNA
gagatcatgtcctaaccctgatcctcttgtcccacagatatccagaaccctctaagaagttcctgctattttaatcttctgtgaatgtggttagagacatgaccctgccgtgtaccagctgagagactctaaatccagtgacaagtctg

SEQ ID NO: 732 - DNA, 149 bp, synthetic construct
  misc_feature 1..149: note = TC15P2_pool2_3
  source 1..149: mol_type = other DNA
gagatcatgtcctaaccctgatcctcttgtcccacagatatccagaaccctctaagaagttcctgctataaataacttcttagaatgtggttagagacatgaccctgccgtgtaccagctgagagactctaaatccagtgacaagtctg

SEQ ID NO: 733 - DNA, 149 bp, synthetic construct
  misc_feature 1..149: note = TC15P2_pool2_4
  source 1..149: mol_type = other DNA
gagatcatgtcctaaccctgatcctcttgtcccacagatatccagaaccctctaagaagttcctgctttatatttgatcaaagaatgtggttagagacatgaccctgccgtgtaccagctgagagactctaaatccagtgacaagtctg

SEQ ID NO: 734 - DNA, 149 bp, synthetic construct
  misc_feature 1..149: note = TC15P2_pool2_5
  source 1..149: mol_type = other DNA
gagatcatgtcctaaccctgatcctcttgtcccacagatatccagaaccctctaagaagttcctgctaatatattgatcacagaatgtggttagagacatgaccctgccgtgtaccagctgagagactctaaatccagtgacaagtctg

SEQ ID NO: 735 - DNA, 149 bp, synthetic construct
  misc_feature 1..149: note = TC15P2_pool2_6
  source 1..149: mol_type = other DNA
gagatcatgtcctaaccctgatcctcttgtcccacagatatccagaaccctctaagaagttcctgcttttaaatagatcagagaatgtggttagagacatgaccctgccgtgtaccagctgagagactctaaatccagtgacaagtctg

SEQ ID NO: 736 - DNA, 149 bp, synthetic construct
  misc_feature 1..149: note = TC15P2_pool2_7
  source 1..149: mol_type = other DNA
gagatcatgtcctaaccctgatcctcttgtcccacagatatccagaaccctctaagaagttcctgctttaaaaaagatcatagaatgtggttagagacatgaccctgccgtgtaccagctgagagactctaaatccagtgacaagtctg

SEQ ID NO: 737 - DNA, 149 bp, synthetic construct
  misc_feature 1..149: note = TC15P2_pool2_8
  source 1..149: mol_type = other DNA
gagatcatgtcctaaccctgatcctcttgtcccacagatatccagaaccctctaagaagttcctgcttattatttgatccatgaatgtggttagagacatgaccctgccgtgtaccagctgagagactctaaatccagtgacaagtctg

SEQ ID NO: 738 - DNA, 149 bp, synthetic construct
  misc_feature 1..149: note = TC15P2_pool2_9
  source 1..149: mol_type = other DNA
gagatcatgtcctaaccctgatcctcttgtcccacagatatccagaaccctctaagaagttcctgctttatttaagatccctgaatgtggttagagacatgaccctgccgtgtaccagctgagagactctaaatccagtgacaagtctg

SEQ ID NO: 739 - DNA, 149 bp, synthetic construct
  misc_feature 1..149: note = TC15P2_pool2_10
  source 1..149: mol_type = other DNA
gagatcatgtcctaaccctgatcctcttgtcccacagatatccagaaccctctaagaagttcctgctaaaataatgatccgagaatgtggttagagacatgaccctgccgtgtaccagctgagagactctaaatccagtgacaagtctg

SEQ ID NO: 740 - DNA, 149 bp, synthetic construct
  misc_feature 1..149: note = TC15P2_pool2_11
  source 1..149: mol_type = other DNA
gagatcatgtcctaaccctgatcctcttgtcccacagatatccagaaccctctaagaagttcctgctatattaaagatccttgaatgtggttagagacatgaccctgccgtgtaccagctgagagactctaaatccagtgacaagtctg

SEQ ID NO: 741 - DNA, 149 bp, synthetic construct
  misc_feature 1..149: note = TC15P2_pool2_12
  source 1..149: mol_type = other DNA
gagatcatgtcctaaccctgatcctcttgtcccacagatatccagaaccctctaagaagttcctgctattaatatgatcgaagaatgtggttagagacatgaccctgccgtgtaccagctgagagactctaaatccagtgacaagtctg

SEQ ID NO: 742 - DNA, 149 bp, synthetic construct
  misc_feature 1..149: note = TC15P2_pool2_13
  source 1..149: mol_type = other DNA
gagatcatgtcctaaccctgatcctcttgtcccacagatatccagaaccctctaagaagttcctgctattatattgatcgctgaatgtggttagagacatgaccctgccgtgtaccagctgagagactctaaatccagtgacaagtctg

SEQ ID NO: 743 - DNA, 149 bp, synthetic construct
  misc_feature 1..149: note = TC15P2_pool2_14
  source 1..149: mol_type = other DNA
gagatcatgtcctaaccctgatcctcttgtcccacagatatccagaaccctctaagaagttcctgctatatatttgatcggtgaatgtggttagagacatgaccctgccgtgtaccagctgagagactctaaatccagtgacaagtctg

SEQ ID NO: 744 - DNA, 149 bp, synthetic construct
  misc_feature 1..149: note = TC15P2_pool2_15
  source 1..149: mol_type = other DNA
gagatcatgtcctaaccctgatcctcttgtcccacagatatccagaaccctctaagaagttcctgctaaaatatagatcgttgaatgtggttagagacatgaccctgccgtgtaccagctgagagactctaaatccagtgacaagtctg

SEQ ID NO: 745 - DNA, 149 bp, synthetic construct
  misc_feature 1..149: note = TC15P2_pool2_16
  source 1..149: mol_type = other DNA
gagatcatgtcctaaccctgatcctcttgtcccacagatatccagaaccctctaagaagttcctgcttataataagatctatgaatgtggttagagacatgaccctgccgtgtaccagctgagagactctaaatccagtgacaagtctg

SEQ ID NO: 746 - DNA, 149 bp, synthetic construct
  misc_feature 1..149: note = TC15P2_pool2_17
  source 1..149: mol_type = other DNA
gagatcatgtcctaaccctgatcctcttgtcccacagatatccagaaccctctaagaagttcctgcttaataaatgatctctgaatgtggttagagacatgaccctgccgtgtaccagctgagagactctaaatccagtgacaagtctg

SEQ ID NO: 747 - DNA, 149 bp, synthetic construct
  misc_feature 1..149: note = TC15P2_pool2_18
  source 1..149: mol_type = other DNA
gagatcatgtcctaaccctgatcctcttgtcccacagatatccagaaccctctaagaagttcctgctaataataagatctgagaatgtggttagagacatgaccctgccgtgtaccagctgagagactctaaatccagtgacaagtctg

SEQ ID NO: 748 - DNA, 149 bp, synthetic construct
  misc_feature 1..149: note = TC15P2_pool2_19
  source 1..149: mol_type = other DNA
gagatcatgtcctaaccctgatcctcttgtcccacagatatccagaaccctctaagaagttcctgctataataatgatctttgaatgtggttagagacatgaccctgccgtgtaccagctgagagactctaaatccagtgacaagtctg

SEQ ID NO: 749 - DNA, 149 bp, synthetic construct
  misc_feature 1..149: note = TC15P2_pool2_20
  source 1..149: mol_type = other DNA
gagatcatgtcctaaccctgatcctcttgtcccacagatatccagaaccctctaagaagttcctgctttattaaagctcaaagaatgtggttagagacatgaccctgccgtgtaccagctgagagactctaaatccagtgacaagtctg

SEQ ID NO: 750 - DNA, 149 bp, synthetic construct
  misc_feature 1..149: note = TC15P2_pool2_21
  source 1..149: mol_type = other DNA
gagatcatgtcctaaccctgatcctcttgtcccacagatatccagaaccctctaagaagttcctgctaattatttgctcacagaatgtggttagagacatgaccctgccgtgtaccagctgagagactctaaatccagtgacaagtctg

SEQ ID NO: 751 - DNA, 149 bp, synthetic construct
  misc_feature 1..149: note = TC15P2_pool2_22
  source 1..149: mol_type = other DNA
gagatcatgtcctaaccctgatcctcttgtcccacagatatccagaaccctctaagaagttcctgctaaataattgctcagagaatgtggttagagacatgaccctgccgtgtaccagctgagagactctaaatccagtgacaagtctg

SEQ ID NO: 752 - DNA, 149 bp, synthetic construct
  misc_feature 1..149: note = TC15P2_pool2_23
  source 1..149: mol_type = other DNA
gagatcatgtcctaaccctgatcctcttgtcccacagatatccagaaccctctaagaagttcctgcttatttaatgctcattgaatgtggttagagacatgaccctgccgtgtaccagctgagagactctaaatccagtgacaagtctg

SEQ ID NO: 753 - DNA, 149 bp, synthetic construct
  misc_feature 1..149: note = TC15P2_pool2_24
  source 1..149: mol_type = other DNA
gagatcatgtcctaaccctgatcctcttgtcccacagatatccagaaccctctaagaagttcctgctttaatttagctccaagaatgtggttagagacatgaccctgccgtgtaccagctgagagactctaaatccagtgacaagtctg

SEQ ID NO: 754 - DNA, 149 bp, synthetic construct
  misc_feature 1..149: note = TC15P2_pool2_25
  source 1..149: mol_type = other DNA
gagatcatgtcctaaccctgatcctcttgtcccacagatatccagaaccctctaagaagttcctgctaatttaaagctccctgaatgtggttagagacatgaccctgccgtgtaccagctgagagactctaaatccagtgacaagtctg

SEQ ID NO: 755 - DNA, 149 bp, synthetic construct
  misc_feature 1..149: note = TC15P2_pool2_26
  source 1..149: mol_type = other DNA
gagatcatgtcctaaccctgatcctcttgtcccacagatatccagaaccctctaagaagttcctgcttatttatagctccgtgaatgtggttagagacatgaccctgccgtgtaccagctgagagactctaaatccagtgacaagtctg

SEQ ID NO: 756 - DNA, 149 bp, synthetic construct
  misc_feature 1..149: note = TC15P2_pool2_27
  source 1..149: mol_type = other DNA
gagatcatgtcctaaccctgatcctcttgtcccacagatatccagaaccctctaagaagttcctgcttttaattagctccttgaatgtggttagagacatgaccctgccgtgtaccagctgagagactctaaatccagtgacaagtctg

SEQ ID NO: 757 - DNA, 149 bp, synthetic construct
  misc_feature 1..149: note = TC15P2_pool2_28
  source 1..149: mol_type = other DNA
gagatcatgtcctaaccctgatcctcttgtcccacagatatccagaaccctctaagaagttcctgctttaatatagctcgatgaatgtggttagagacatgaccctgccgtgtaccagctgagagactctaaatccagtgacaagtctg

SEQ ID NO: 758 - DNA, 149 bp, synthetic construct
  misc_feature 1..149: note = TC15P2_pool2_29
  source 1..149: mol_type = other DNA
gagatcatgtcctaaccctgatcctcttgtcccacagatatccagaaccctctaagaagttcctgctaaatttaagctcgcagaatgtggttagagacatgaccctgccgtgtaccagctgagagactctaaatccagtgacaagtctg

SEQ ID NO: 759 - DNA, 149 bp, synthetic construct
  misc_feature 1..149: note = TC15P2_pool2_30
  source 1..149: mol_type = other DNA
gagatcatgtcctaaccctgatcctcttgtcccacagatatccagaaccctctaagaagttcctgctataaaaaagctcggtgaatgtggttagagacatgaccctgccgtgtaccagctgagagactctaaatccagtgacaagtctg

SEQ ID NO: 760 - DNA, 149 bp, synthetic construct
  misc_feature 1..149: note = TC15P2_pool2_31
  source 1..149: mol_type = other DNA
gagatcatgtcctaaccctgatcctcttgtcccacagatatccagaaccctctaagaagttcctgctttaataatgctcgtagaatgtggttagagacatgaccctgccgtgtaccagctgagagactctaaatccagtgacaagtctg

SEQ ID NO: 761 - DNA, 149 bp, synthetic construct
  misc_feature 1..149: note = TC15P2_pool2_32
  source 1..149: mol_type = other DNA
gagatcatgtcctaaccctgatcctcttgtcccacagatatccagaaccctctaagaagttcctgctattaataagctctatgaatgtggttagagacatgaccctgccgtgtaccagctgagagactctaaatccagtgacaagtctg

SEQ ID NO: 762 - DNA, 149 bp, synthetic construct
  misc_feature 1..149: note = TC15P2_pool2_33
  source 1..149: mol_type = other DNA
gagatcatgtcctaaccctgatcctcttgtcccacagatatccagaaccctctaagaagttcctgctatattaatgctctcagaatgtggttagagacatgaccctgccgtgtaccagctgagagactctaaatccagtgacaagtctg

SEQ ID NO: 763 - DNA, 149 bp, synthetic construct
  misc_feature 1..149: note = TC15P2_pool2_34
  source 1..149: mol_type = other DNA
gagatcatgtcctaaccctgatcctcttgtcccacagatatccagaaccctctaagaagttcctgctttataattgctctgtgaatgtggttagagacatgaccctgccgtgtaccagctgagagactctaaatccagtgacaagtctg

SEQ ID NO: 764 - DNA, 149 bp, synthetic construct
  misc_feature 1..149: note = TC15P2_pool2_35
  source 1..149: mol_type = other DNA
gagatcatgtcctaaccctgatcctcttgtcccacagatatccagaaccctctaagaagttcctgcttatatattgctctttgaatgtggttagagacatgaccctgccgtgtaccagctgagagactctaaatccagtgacaagtctg

SEQ ID NO: 765 - DNA, 149 bp, synthetic construct
  misc_feature 1..149: note = TC15P2_pool2_36
  source 1..149: mol_type = other DNA
gagatcatgtcctaaccctgatcctcttgtcccacagatatccagaaccctctaagaagttcctgcttatattttggtcaaagaatgtggttagagacatgaccctgccgtgtaccagctgagagactctaaatccagtgacaagtctg

SEQ ID NO: 766 - DNA, 149 bp, synthetic construct
  misc_feature 1..149: note = TC15P2_pool2_37
  source 1..149: mol_type = other DNA
gagatcatgtcctaaccctgatcctcttgtcccacagatatccagaaccctctaagaagttcctgcttaatattaggtcactgaatgtggttagagacatgaccctgccgtgtaccagctgagagactctaaatccagtgacaagtctg

SEQ ID NO: 767 - DNA, 149 bp, synthetic construct
  misc_feature 1..149: note = TC15P2_pool2_38
  source 1..149: mol_type = other DNA
gagatcatgtcctaaccctgatcctcttgtcccacagatatccagaaccctctaagaagttcctgcttaaaatttggtcagagaatgtggttagagacatgaccctgccgtgtaccagctgagagactctaaatccagtgacaagtctg

SEQ ID NO: 768 - DNA, 149 bp, synthetic construct
  misc_feature 1..149: note = TC15P2_pool2_39
  source 1..149: mol_type = other DNA
gagatcatgtcctaaccctgatcctcttgtcccacagatatccagaaccctctaagaagttcctgctatataataggtcattgaatgtggttagagacatgaccctgccgtgtaccagctgagagactctaaatccagtgacaagtctg

SEQ ID NO: 769 - DNA, 149 bp, synthetic construct
  misc_feature 1..149: note = TC15P2_pool2_40
  source 1..149: mol_type = other DNA
gagatcatgtcctaaccctgatcctcttgtcccacagatatccagaaccctctaagaagttcctgctaattaaaaggtccaagaatgtggttagagacatgaccctgccgtgtaccagctgagagactctaaatccagtgacaagtctg

SEQ ID NO: 770 - DNA, 149 bp, synthetic construct
  misc_feature 1..149: note = TC15P2_pool2_41
  source 1..149: mol_type = other DNA
gagatcatgtcctaaccctgatcctcttgtcccacagatatccagaaccctctaagaagttcctgctaataaaatggtccctgaatgtggttagagacatgaccctgccgtgtaccagctgagagactctaaatccagtgacaagtctg

SEQ ID NO: 771 - DNA, 149 bp, synthetic construct
  misc_feature 1..149: note = TC15P2_pool2_42
  source 1..149: mol_type = other DNA
gagatcatgtcctaaccctgatcctcttgtcccacagatatccagaaccctctaagaagttcctgctaaaaataaggtccgtgaatgtggttagagacatgaccctgccgtgtaccagctgagagactctaaatccagtgacaagtctg

SEQ ID NO: 772 - DNA, 149 bp, synthetic construct
  misc_feature 1..149: note = TC15P2_pool2_43
  source 1..149: mol_type = other DNA
gagatcatgtcctaaccctgatcctcttgtcccacagatatccagaaccctctaagaagttcctgctatttttaaggtccttgaatgtggttagagacatgaccctgccgtgtaccagctgagagactctaaatccagtgacaagtctg

SEQ ID NO: 773 - DNA, 149 bp, synthetic construct
  misc_feature 1..149: note = TC15P2_pool2_44
  source 1..149: mol_type = other DNA
gagatcatgtcctaaccctgatcctcttgtcccacagatatccagaaccctctaagaagttcctgcttttttaaaggtcgatgaatgtggttagagacatgaccctgccgtgtaccagctgagagactctaaatccagtgacaagtctg

SEQ ID NO: 774 - DNA, 149 bp, synthetic construct
  misc_feature 1..149: note = TC15P2_pool2_45
  source 1..149: mol_type = other DNA
gagatcatgtcctaaccctgatcctcttgtcccacagatatccagaaccctctaagaagttcctgctataattatggtcgcagaatgtggttagagacatgaccctgccgtgtaccagctgagagactctaaatccagtgacaagtctg

SEQ ID NO: 775 - DNA, 149 bp, synthetic construct
  misc_feature 1..149: note = TC15P2_pool2_46
  source 1..149: mol_type = other DNA
gagatcatgtcctaaccctgatcctcttgtcccacagatatccagaaccctctaagaagttcctgctaataaataggtcggagaatgtggttagagacatgaccctgccgtgtaccagctgagagactctaaatccagtgacaagtctg

SEQ ID NO: 776 - DNA, 149 bp, synthetic construct
  misc_feature 1..149: note = TC15P2_pool2_47
  source 1..149: mol_type = other DNA
gagatcatgtcctaaccctgatcctcttgtcccacagatatccagaaccctctaagaagttcctgcttatataatggtcgtagaatgtggttagagacatgaccctgccgtgtaccagctgagagactctaaatccagtgacaagtctg

SEQ ID NO: 777 - DNA, 149 bp, synthetic construct
  misc_feature 1..149: note = TC15P2_pool2_48
  source 1..149: mol_type = other DNA
gagatcatgtcctaaccctgatcctcttgtcccacagatatccagaaccctctaagaagttcctgcttaaaaaaaggtctatgaatgtggttagagacatgaccctgccgtgtaccagctgagagactctaaatccagtgacaagtctg

SEQ ID NO: 778 - DNA, 149 bp, synthetic construct
  misc_feature 1..149: note = TC15P2_pool2_49
  source 1..149: mol_type = other DNA
gagatcatgtcctaaccctgatcctcttgtcccacagatatccagaaccctctaagaagttcctgctatttaattggtctcagaatgtggttagagacatgaccctgccgtgtaccagctgagagactctaaatccagtgacaagtctg

SEQ ID NO: 779 - DNA, 149 bp, synthetic construct
  misc_feature 1..149: note = TC15P2_pool2_50
  source 1..149: mol_type = other DNA
gagatcatgtcctaaccctgatcctcttgtcccacagatatccagaaccctctaagaagttcctgctttataataggtctgagaatgtggttagagacatgaccctgccgtgtaccagctgagagactctaaatccagtgacaagtctg

SEQ ID NO: 780 - DNA, 149 bp, synthetic construct
  misc_feature 1..149: note = TC15P2_pool2_51
  source 1..149: mol_type = other DNA
gagatcatgtcctaaccctgatcctcttgtcccacagatatccagaaccctctaagaagttcctgcttatatataggtcttagaatgtggttagagacatgaccctgccgtgtaccagctgagagactctaaatccagtgacaagtctg

SEQ ID NO: 781 - DNA, 149 bp, synthetic construct
  misc_feature 1..149: note = TC15P2_pool2_52
  source 1..149: mol_type = other DNA
gagatcatgtcctaaccctgatcctcttgtcccacagatatccagaaccctctaagaagttcctgctaaaaaaaagttcaaagaatgtggttagagacatgaccctgccgtgtaccagctgagagactctaaatccagtgacaagtctg

SEQ ID NO: 782 - DNA, 149 bp, synthetic construct
  misc_feature 1..149: note = TC15P2_pool2_53
  source 1..149: mol_type = other DNA
gagatcatgtcctaaccctgatcctcttgtcccacagatatccagaaccctctaagaagttcctgcttattttaagttcacagaatgtggttagagacatgaccctgccgtgtaccagctgagagactctaaatccagtgacaagtctg

SEQ ID NO: 783 - DNA, 149 bp, synthetic construct
  misc_feature 1..149: note = TC15P2_pool2_54
  source 1..149: mol_type = other DNA
gagatcatgtcctaaccctgatcctcttgtcccacagatatccagaaccctctaagaagttcctgctaatttttagttcagagaatgtggttagagacatgaccctgccgtgtaccagctgagagactctaaatccagtgacaagtctg

SEQ ID NO: 784 - DNA, 149 bp, synthetic construct
  misc_feature 1..149: note = TC15P2_pool2_55
  source 1..149: mol_type = other DNA
gagatcatgtcctaaccctgatcctcttgtcccacagatatccagaaccctctaagaagttcctgctaaattatagttcatagaatgtggttagagacatgaccctgccgtgtaccagctgagagactctaaatccagtgacaagtctg

SEQ ID NO: 785 - DNA, 149 bp, synthetic construct
  misc_feature 1..149: note = TC15P2_pool2_56
  source 1..149: mol_type = other DNA
gagatcatgtcctaaccctgatcctcttgtcccacagatatccagaaccctctaagaagttcctgcttttaaattgttccatgaatgtggttagagacatgaccctgccgtgtaccagctgagagactctaaatccagtgacaagtctg

SEQ ID NO: 786 - DNA, 149 bp, synthetic construct
  misc_feature 1..149: note = TC15P2_pool2_57
  source 1..149: mol_type = other DNA
gagatcatgtcctaaccctgatcctcttgtcccacagatatccagaaccctctaagaagttcctgcttttaatttgttcccagaatgtggttagagacatgaccctgccgtgtaccagctgagagactctaaatccagtgacaagtctg

SEQ ID NO: 787 - DNA, 149 bp, synthetic construct
  misc_feature 1..149: note = TC15P2_pool2_58
  source 1..149: mol_type = other DNA
gagatcatgtcctaaccctgatcctcttgtcccacagatatccagaaccctctaagaagttcctgctataattaagttccgtgaatgtggttagagacatgaccctgccgtgtaccagctgagagactctaaatccagtgacaagtctg

SEQ ID NO: 788 - DNA, 149 bp, synthetic construct
  misc_feature 1..149: note = TC15P2_pool2_59
  source 1..149: mol_type = other DNA
gagatcatgtcctaaccctgatcctcttgtcccacagatatccagaaccctctaagaagttcctgctttaaaattgttcctagaatgtggttagagacatgaccctgccgtgtaccagctgagagactctaaatccagtgacaagtctg

SEQ ID NO: 789 - DNA, 149 bp, synthetic construct
  misc_feature 1..149: note = TC15P2_pool2_60
  source 1..149: mol_type = other DNA
gagatcatgtcctaaccctgatcctcttgtcccacagatatccagaaccctctaagaagttcctgctttatttttgttcgatgaatgtggttagagacatgaccctgccgtgtaccagctgagagactctaaatccagtgacaagtctg

SEQ ID NO: 790 - DNA, 149 bp, synthetic construct
  misc_feature 1..149: note = TC15P2_pool2_61
  source 1..149: mol_type = other DNA
gagatcatgtcctaaccctgatcctcttgtcccacagatatccagaaccctctaagaagttcctgcttaattaaagttcgctgaatgtggttagagacatgaccctgccgtgtaccagctgagagactctaaatccagtgacaagtctg

SEQ ID NO: 791 - DNA, 149 bp, synthetic construct
  misc_feature 1..149: note = TC15P2_pool2_62
  source 1..149: mol_type = other DNA
gagatcatgtcctaaccctgatcctcttgtcccacagatatccagaaccctctaagaagttcctgcttataaaaagttcggagaatgtggttagagacatgaccctgccgtgtaccagctgagagactctaaatccagtgacaagtctg

SEQ ID NO: 792 - DNA, 149 bp, synthetic construct
  misc_feature 1..149: note = TC15P2_pool2_63
  source 1..149: mol_type = other DNA
gagatcatgtcctaaccctgatcctcttgtcccacagatatccagaaccctctaagaagttcctgcttatatttagttcgttgaatgtggttagagacatgaccctgccgtgtaccagctgagagactctaaatccagtgacaagtctg

SEQ ID NO: 793 - DNA, 149 bp, synthetic construct
  misc_feature 1..149: note = TC15P2_pool2_64
  source 1..149: mol_type = other DNA
gagatcatgtcctaaccctgatcctcttgtcccacagatatccagaaccctctaagaagttcctgcttaaataatgttctatgaatgtggttagagacatgaccctgccgtgtaccagctgagagactctaaatccagtgacaagtctg

SEQ ID NO: 794 - DNA, 149 bp, synthetic construct
  misc_feature 1..149: note = TC15P2_pool2_65
  source 1..149: mol_type = other DNA
gagatcatgtcctaaccctgatcctcttgtcccacagatatccagaaccctctaagaagttcctgctaattttatgttctcagaatgtggttagagacatgaccctgccgtgtaccagctgagagactctaaatccagtgacaagtctg

SEQ ID NO: 795 - DNA, 149 bp, synthetic construct
  misc_feature 1..149: note = TC15P2_pool2_66
  source 1..149: mol_type = other DNA
gagatcatgtcctaaccctgatcctcttgtcccacagatatccagaaccctctaagaagttcctgctatttattagttctgtgaatgtggttagagacatgaccctgccgtgtaccagctgagagactctaaatccagtgacaagtctg

SEQ ID NO: 796 - DNA, 149 bp, synthetic construct
  misc_feature 1..149: note = TC15P2_pool2_67
  source 1..149: mol_type = other DNA
gagatcatgtcctaaccctgatcctcttgtcccacagatatccagaaccctctaagaagttcctgctaattttttgttctttgaatgtggttagagacatgaccctgccgtgtaccagctgagagactctaaatccagtgacaagtctg

SEQ ID NO: 797 - DNA, 149 bp, synthetic construct
  misc_feature 1..149: note = TC15P3_pool2_68
  source 1..149: mol_type = other DNA
gagatcatgtcctaaccctgatcctcttgtcccacagatatccagaaccctctaagaagttcctgctttttaatatatcaatgaatgtggttagagacatgaccctgccgtgtaccagctgagagactctaaatccagtgacaagtctg

SEQ ID NO: 798 - DNA, 149 bp, synthetic construct
  misc_feature 1..149: note = TC15P3_pool2_69
  source 1..149: mol_type = other DNA
gagatcatgtcctaaccctgatcctcttgtcccacagatatccagaaccctctaagaagttcctgctaaattttatatcactgaatgtggttagagacatgaccctgccgtgtaccagctgagagactctaaatccagtgacaagtctg

SEQ ID NO: 799 - DNA, 149 bp, synthetic construct
  misc_feature 1..149: note = TC15P3_pool2_70
  source 1..149: mol_type = other DNA
gagatcatgtcctaaccctgatcctcttgtcccacagatatccagaaccctctaagaagttcctgctttttaaattatcagtgaatgtggttagagacatgaccctgccgtgtaccagctgagagactctaaatccagtgacaagtctg

SEQ ID NO: 800 - DNA, 149 bp, synthetic construct
  misc_feature 1..149: note = TC15P3_pool2_71
  source 1..149: mol_type = other DNA
gagatcatgtcctaaccctgatcctcttgtcccacagatatccagaaccctctaagaagttcctgctatattttttatcatagaatgtggttagagacatgaccctgccgtgtaccagctgagagactctaaatccagtgacaagtctg

SEQ ID NO: 801 - DNA, 149 bp, synthetic construct
  misc_feature 1..149: note = TC15P3_pool2_72
  source 1..149: mol_type = other DNA
gagatcatgtcctaaccctgatcctcttgtcccacagatatccagaaccctctaagaagttcctgcttataaatttatccaagaatgtggttagagacatgaccctgccgtgtaccagctgagagactctaaatccagtgacaagtctg

SEQ ID NO: 802 - DNA, 149 bp, synthetic construct
  misc_feature 1..149: note = TC15P3_pool2_73
  source 1..149: mol_type = other DNA
gagatcatgtcctaaccctgatcctcttgtcccacagatatccagaaccctctaagaagttcctgctaatattaatatccctgaatgtggttagagacatgaccctgccgtgtaccagctgagagactctaaatccagtgacaagtctg

SEQ ID NO: 803 - DNA, 149 bp, synthetic construct
  misc_feature 1..149: note = TC15P3_pool2_74
  source 1..149: mol_type = other DNA
gagatcatgtcctaaccctgatcctcttgtcccacagatatccagaaccctctaagaagttcctgctaaatttattatccgtgaatgtggttagagacatgaccctgccgtgtaccagctgagagactctaaatccagtgacaagtctg

SEQ ID NO: 804 - DNA, 149 bp, synthetic construct
  misc_feature 1..149: note = TC15P3_pool2_75
  source 1..149: mol_type = other DNA
gagatcatgtcctaaccctgatcctcttgtcccacagatatccagaaccctctaagaagttcctgctttataaaatatccttgaatgtggttagagacatgaccctgccgtgtaccagctgagagactctaaatccagtgacaagtctg

SEQ ID NO: 805 - DNA, 149 bp, synthetic construct
  misc_feature 1..149: note = TC15P3_pool2_76
  source 1..149: mol_type = other DNA
gagatcatgtcctaaccctgatcctcttgtcccacagatatccagaaccctctaagaagttcctgcttaaaaatatatcgaagaatgtggttagagacatgaccctgccgtgtaccagctgagagactctaaatccagtgacaagtctg

SEQ ID NO: 806 - DNA, 149 bp, synthetic construct
  misc_feature 1..149: note = TC15P3_pool2_77
  source 1..149: mol_type = other DNA
gagatcatgtcctaaccctgatcctcttgtcccacagatatccagaaccctctaagaagttcctgctattaattttatcgctgaatgtggttagagacatgaccctgccgtgtaccagctgagagactctaaatccagtgacaagtctg

SEQ ID NO: 807 - DNA, 149 bp, synthetic construct
  misc_feature 1..149: note = TC15P3_pool2_78
  source 1..149: mol_type = other DNA
gagatcatgtcctaaccctgatcctcttgtcccacagatatccagaaccctctaagaagttcctgcttatataaatatcggtgaatgtggttagagacatgaccctgccgtgtaccagctgagagactctaaatccagtgacaagtctg

SEQ ID NO: 808 - DNA, 149 bp, synthetic construct
  misc_feature 1..149: note = TC15P3_pool2_79
  source 1..149: mol_type = other DNA
gagatcatgtcctaaccctgatcctcttgtcccacagatatccagaaccctctaagaagttcctgctataaattatatcgttgaatgtggttagagacatgaccctgccgtgtaccagctgagagactctaaatccagtgacaagtctg

SEQ ID NO: 809 - DNA, 149 bp, synthetic construct
  misc_feature 1..149: note = TC15P3_pool2_80
  source 1..149: mol_type = other DNA
gagatcatgtcctaaccctgatcctcttgtcccacagatatccagaaccctctaagaagttcctgcttttattattatctatgaatgtggttagagacatgaccctgccgtgtaccagctgagagactctaaatccagtgacaagtctg

SEQ ID NO: 810 - DNA, 149 bp, synthetic construct
  misc_feature 1..149: note = TC15P3_pool2_81
  source 1..149: mol_type = other DNA
gagatcatgtcctaaccctgatcctcttgtcccacagatatccagaaccctctaagaagttcctgctataaattttatctcagaatgtggttagagacatgaccctgccgtgtaccagctgagagactctaaatccagtgacaagtctg

SEQ ID NO: 811 - DNA, 149 bp, synthetic construct
  misc_feature 1..149: note = TC15P3_pool2_82
  source 1..149: mol_type = other DNA
gagatcatgtcctaaccctgatcctcttgtcccacagatatccagaaccctctaagaagttcctgctattatttatatctgtgaatgtggttagagacatgaccctgccgtgtaccagctgagagactctaaatccagtgacaagtctg

SEQ ID NO: 812 - DNA, 149 bp, synthetic construct
  misc_feature 1..149: note = TC15P3_pool2_83
  source 1..149: mol_type = other DNA
gagatcatgtcctaaccctgatcctcttgtcccacagatatccagaaccctctaagaagttcctgctaaataaaatatctttgaatgtggttagagacatgaccctgccgtgtaccagctgagagactctaaatccagtgacaagtctg

SEQ ID NO: 813 - DNA, 149 bp, synthetic construct
  misc_feature 1..149: note = TC15P3_pool2_84
  source 1..149: mol_type = other DNA
gagatcatgtcctaaccctgatcctcttgtcccacagatatccagaaccctctaagaagttcctgcttaattaattctcaaagaatgtggttagagacatgaccctgccgtgtaccagctgagagactctaaatccagtgacaagtctg

SEQ ID NO: 814 - DNA, 149 bp, synthetic construct
  misc_feature 1..149: note = TC15P3_pool2_85
  source 1..149: mol_type = other DNA
gagatcatgtcctaaccctgatcctcttgtcccacagatatccagaaccctctaagaagttcctgctaattataatctcactgaatgtggttagagacatgaccctgccgtgtaccagctgagagactctaaatccagtgacaagtctg

SEQ ID NO: 815 - DNA, 149 bp, synthetic construct
  misc_feature 1..149: note = TC15P3_pool2_86
  source 1..149: mol_type = other DNA
gagatcatgtcctaaccctgatcctcttgtcccacagatatccagaaccctctaagaagttcctgctatattatttctcagtgaatgtggttagagacatgaccctgccgtgtaccagctgagagactctaaatccagtgacaagtctg

SEQ ID NO: 816 - DNA, 149 bp, synthetic construct
  misc_feature 1..149: note = TC15P3_pool2_87
  source 1..149: mol_type = other DNA
gagatcatgtcctaaccctgatcctcttgtcccacagatatccagaaccctctaagaagttcctgctttataaattctcatagaatgtggttagagacatgaccctgccgtgtaccagctgagagactctaaatccagtgacaagtctg

SEQ ID NO: 817 - DNA, 149 bp, synthetic construct
  misc_feature 1..149: note = TC15P3_pool2_88
  source 1..149: mol_type = other DNA
gagatcatgtcctaaccctgatcctcttgtcccacagatatccagaaccctctaagaagttcctgctttttatattctccaagaatgtggttagagacatgaccctgccgtgtaccagctgagagactctaaatccagtgacaagtctg

SEQ ID NO: 818 - DNA, 149 bp, synthetic construct
  misc_feature 1..149: note = TC15P3_pool2_89
  source 1..149: mol_type = other DNA
gagatcatgtcctaaccctgatcctcttgtcccacagatatccagaaccctctaagaagttcctgctaaaatttttctcccagaatgtggttagagacatgaccctgccgtgtaccagctgagagactctaaatccagtgacaagtctg

SEQ ID NO: 819 - DNA, 149 bp, synthetic construct
  misc_feature 1..149: note = TC15P3_pool2_90
  source 1..149: mol_type = other DNA
gagatcatgtcctaaccctgatcctcttgtcccacagatatccagaaccctctaagaagttcctgcttattaaaatctccgtgaatgtggttagagacatgaccctgccgtgtaccagctgagagactctaaatccagtgacaagtctg

SEQ ID NO: 820 - DNA, 149 bp, synthetic construct
  misc_feature 1..149: note = TC15P3_pool2_91
  source 1..149: mol_type = other DNA
gagatcatgtcctaaccctgatcctcttgtcccacagatatccagaaccctctaagaagttcctgcttaaattaatctcctagaatgtggttagagacatgaccctgccgtgtaccagctgagagactctaaatccagtgacaagtctg

SEQ ID NO: 821 - DNA, 149 bp, synthetic construct
  misc_feature 1..149: note = TC15P3_pool2_92
  source 1..149: mol_type = other DNA
gagatcatgtcctaaccctgatcctcttgtcccacagatatccagaaccctctaagaagttcctgcttaaatatttctcgaagaatgtggttagagacatgaccctgccgtgtaccagctgagagactctaaatccagtgacaagtctg

SEQ ID NO: 822 - DNA, 149 bp, synthetic construct
  misc_feature 1..149: note = TC15P3_pool2_93
  source 1..149: mol_type = other DNA
gagatcatgtcctaaccctgatcctcttgtcccacagatatccagaaccctctaagaagttcctgctaataattatctcgctgaatgtggttagagacatgaccctgccgtgtaccagctgagagactctaaatccagtgacaagtctg

SEQ ID NO: 823 - DNA, 149 bp, synthetic construct
  misc_feature 1..149: note = TC15P3_pool2_94
  source 1..149: mol_type = other DNA
gagatcatgtcctaaccctgatcctcttgtcccacagatatccagaaccctctaagaagttcctgctatataaattctcggtgaatgtggttagagacatgaccctgccgtgtaccagctgagagactctaaatccagtgacaagtctg

SEQ ID NO: 824 - DNA, 149 bp, synthetic construct
  misc_feature 1..149: note = TC15P3_pool2_95
  source 1..149: mol_type = other DNA
gagatcatgtcctaaccctgatcctcttgtcccacagatatccagaaccctctaagaagttcctgctttaaatattctcgttgaatgtggttagagacatgaccctgccgtgtaccagctgagagactctaaatccagtgacaagtctg

SEQ ID NO: 825 - DNA, 149 bp, synthetic construct
  misc_feature 1..149: note = TC15P3_pool2_96
  source 1..149: mol_type = other DNA
gagatcatgtcctaaccctgatcctcttgtcccacagatatccagaaccctctaagaagttcctgctaaaaatattctctaagaatgtggttagagacatgaccctgccgtgtaccagctgagagactctaaatccagtgacaagtctg

SEQ ID NO: 826 - DNA, 149 bp, synthetic construct
  misc_feature 1..149: note = TC15P3_pool2_97
  source 1..149: mol_type = other DNA
gagatcatgtcctaaccctgatcctcttgtcccacagatatccagaaccctctaagaagttcctgctatatatattctctcagaatgtggttagagacatgaccctgccgtgtaccagctgagagactctaaatccagtgacaagtctg

SEQ ID NO: 827 - DNA, 149 bp, synthetic construct
  misc_feature 1..149: note = TC15P3_pool2_98
  source 1..149: mol_type = other DNA
gagatcatgtcctaaccctgatcctcttgtcccacagatatccagaaccctctaagaagttcctgctttattaattctctgtgaatgtggttagagacatgaccctgccgtgtaccagctgagagactctaaatccagtgacaagtctg

SEQ ID NO: 828 - DNA, 149 bp, synthetic construct
  misc_feature 1..149: note = TC15P3_pool2_99
  source 1..149: mol_type = other DNA
gagatcatgtcctaaccctgatcctcttgtcccacagatatccagaaccctctaagaagttcctgctattataaatctcttagaatgtggttagagacatgaccctgccgtgtaccagctgagagactctaaatccagtgacaagtctg

SEQ ID NO: 829 - DNA, 149 bp, synthetic construct
  misc_feature 1..149: note = TC15P3_pool2_100
  source 1..149: mol_type = other DNA
gagatcatgtcctaaccctgatcctcttgtcccacagatatccagaaccctctaagaagttcctgcttttaaaaatgtcaatgaatgtggttagagacatgaccctgccgtgtaccagctgagagactctaaatccagtgacaagtctg

SEQ ID NO: 830 - DNA, 149 bp, synthetic construct
  misc_feature 1..149: note = TC15P3_pool2_101
  source 1..149: mol_type = other DNA
gagatcatgtcctaaccctgatcctcttgtcccacagatatccagaaccctctaagaagttcctgcttataaatatgtcactgaatgtggttagagacatgaccctgccgtgtaccagctgagagactctaaatccagtgacaagtctg

SEQ ID NO: 831 - DNA, 149 bp, synthetic construct
  misc_feature 1..149: note = TC15P3_pool2_102
  source 1..149: mol_type = other DNA
gagatcatgtcctaaccctgatcctcttgtcccacagatatccagaaccctctaagaagttcctgcttttaataatgtcagagaatgtggttagagacatgaccctgccgtgtaccagctgagagactctaaatccagtgacaagtctg

SEQ ID NO: 832 - DNA, 149 bp, synthetic construct
  misc_feature 1..149: note = TC15P3_pool2_103
  source 1..149: mol_type = other DNA
gagatcatgtcctaaccctgatcctcttgtcccacagatatccagaaccctctaagaagttcctgctataatttttgtcattgaatgtggttagagacatgaccctgccgtgtaccagctgagagactctaaatccagtgacaagtctg

SEQ ID NO: 833 - DNA, 149 bp, synthetic construct
  misc_feature 1..149: note = TC15P3_pool2_104
  source 1..149: mol_type = other DNA
gagatcatgtcctaaccctgatcctcttgtcccacagatatccagaaccctctaagaagttcctgcttttttatttgtccaagaatgtggttagagacatgaccctgccgtgtaccagctgagagactctaaatccagtgacaagtctg

SEQ ID NO: 834 - DNA, 149 bp, synthetic construct
  misc_feature 1..149: note = TC15P3_pool2_105
  source 1..149: mol_type = other DNA
gagatcatgtcctaaccctgatcctcttgtcccacagatatccagaaccctctaagaagttcctgcttaattatttgtccctgaatgtggttagagacatgaccctgccgtgtaccagctgagagactctaaatccagtgacaagtctg

SEQ ID NO: 835 - DNA, 149 bp, synthetic construct
  misc_feature 1..149: note = TC15P3_pool2_106
  source 1..149: mol_type = other DNA
gagatcatgtcctaaccctgatcctcttgtcccacagatatccagaaccctctaagaagttcctgctataaaaattgtccgagaatgtggttagagacatgaccctgccgtgtaccagctgagagactctaaatccagtgacaagtctg

SEQ ID NO: 836 - DNA, 149 bp, synthetic construct
  misc_feature 1..149: note = TC15P3_pool2_107
  source 1..149: mol_type = other DNA
gagatcatgtcctaaccctgatcctcttgtcccacagatatccagaaccctctaagaagttcctgcttataatattgtcctagaatgtggttagagacatgaccctgccgtgtaccagctgagagactctaaatccagtgacaagtctg

SEQ ID NO: 837 - DNA, 149 bp, synthetic construct
  misc_feature 1..149: note = TC15P3_pool2_108
  source 1..149: mol_type = other DNA
gagatcatgtcctaaccctgatcctcttgtcccacagatatccagaaccctctaagaagttcctgcttaatatattgtcgaagaatgtggttagagacatgaccctgccgtgtaccagctgagagactctaaatccagtgacaagtctg

SEQ ID NO: 838 - DNA, 149 bp, synthetic construct
  misc_feature 1..149: note = TC15P3_pool2_109
  source 1..149: mol_type = other DNA
gagatcatgtcctaaccctgatcctcttgtcccacagatatccagaaccctctaagaagttcctgcttaattttttgtcgcagaatgtggttagagacatgaccctgccgtgtaccagctgagagactctaaatccagtgacaagtctg

SEQ ID NO: 839 - DNA, 149 bp, synthetic construct
  misc_feature 1..149: note = TC15P3_pool2_110
  source 1..149: mol_type = other DNA
gagatcatgtcctaaccctgatcctcttgtcccacagatatccagaaccctctaagaagttcctgctatttaaaatgtcggtgaatgtggttagagacatgaccctgccgtgtaccagctgagagactctaaatccagtgacaagtctg

SEQ ID NO: 840 - DNA, 149 bp, synthetic construct
  misc_feature 1..149: note = TC15P3_pool2_111
  source 1..149: mol_type = other DNA
gagatcatgtcctaaccctgatcctcttgtcccacagatatccagaaccctctaagaagttcctgctatataaaatgtcgtagaatgtggttagagacatgaccctgccgtgtaccagctgagagactctaaatccagtgacaagtctg

SEQ ID NO: 841 - DNA, 149 bp, synthetic construct
  misc_feature 1..149: note = TC15P3_pool2_112
  source 1..149: mol_type = other DNA
gagatcatgtcctaaccctgatcctcttgtcccacagatatccagaaccctctaagaagttcctgctataatatttgtctaagaatgtggttagagacatgaccctgccgtgtaccagctgagagactctaaatccagtgacaagtctg

SEQ ID NO: 842 - DNA, 149 bp, synthetic construct
  misc_feature 1..149: note = TC15P3_pool2_113
  source 1..149: mol_type = other DNA
gagatcatgtcctaaccctgatcctcttgtcccacagatatccagaaccctctaagaagttcctgctattatttttgtctcagaatgtggttagagacatgaccctgccgtgtaccagctgagagactctaaatccagtgacaagtctg

SEQ ID NO: 843 - DNA, 149 bp, synthetic construct
  misc_feature 1..149: note = TC15P3_pool2_114
  source 1..149: mol_type = other DNA
gagatcatgtcctaaccctgatcctcttgtcccacagatatccagaaccctctaagaagttcctgctaattaatatgtctgtgaatgtggttagagacatgaccctgccgtgtaccagctgagagactctaaatccagtgacaagtctg

SEQ ID NO: 844 - DNA, 149 bp, synthetic construct
  misc_feature 1..149: note = TC15P3_pool2_115
  source 1..149: mol_type = other DNA
gagatcatgtcctaaccctgatcctcttgtcccacagatatccagaaccctctaagaagttcctgcttattattatgtcttagaatgtggttagagacatgaccctgccgtgtaccagctgagagactctaaatccagtgacaagtctg

SEQ ID NO: 845 - DNA, 149 bp, synthetic construct
  misc_feature 1..149: note = TC15P3_pool2_116
  source 1..149: mol_type = other DNA
gagatcatgtcctaaccctgatcctcttgtcccacagatatccagaaccctctaagaagttcctgctattaaatatttcaatgaatgtggttagagacatgaccctgccgtgtaccagctgagagactctaaatccagtgacaagtctg

SEQ ID NO: 846 - DNA, 149 bp, synthetic construct
  misc_feature 1..149: note = TC15P3_pool2_117
  source 1..149: mol_type = other DNA
gagatcatgtcctaaccctgatcctcttgtcccacagatatccagaaccctctaagaagttcctgctaaaaaatttttcactgaatgtggttagagacatgaccctgccgtgtaccagctgagagactctaaatccagtgacaagtctg

SEQ ID NO: 847 - DNA, 149 bp, synthetic construct
  misc_feature 1..149: note = TC15P3_pool2_118
  source 1..149: mol_type = other DNA
gagatcatgtcctaaccctgatcctcttgtcccacagatatccagaaccctctaagaagttcctgctttttaaaatttcagagaatgtggttagagacatgaccctgccgtgtaccagctgagagactctaaatccagtgacaagtctg

SEQ ID NO: 848 - DNA, 149 bp, synthetic construct
  misc_feature 1..149: note = TC15P3_pool2_119
  source 1..149: mol_type = other DNA
gagatcatgtcctaaccctgatcctcttgtcccacagatatccagaaccctctaagaagttcctgcttaaatttttttcattgaatgtggttagagacatgaccctgccgtgtaccagctgagagactctaaatccagtgacaagtctg

SEQ ID NO: 849 - DNA, 149 bp, synthetic construct
  misc_feature 1..149: note = TC15P3_pool2_120
  source 1..149: mol_type = other DNA
gagatcatgtcctaaccctgatcctcttgtcccacagatatccagaaccctctaagaagttcctgctttatttattttccaagaatgtggttagagacatgaccctgccgtgtaccagctgagagactctaaatccagtgacaagtctg

SEQ ID NO: 850 - DNA, 149 bp, synthetic construct
  misc_feature 1..149: note = TC15P3_pool2_121
  source 1..149: mol_type = other DNA
gagatcatgtcctaaccctgatcctcttgtcccacagatatccagaaccctctaagaagttcctgctataaaatatttcccagaatgtggttagagacatgaccctgccgtgtaccagctgagagactctaaatccagtgacaagtctg

SEQ ID NO: 851 - DNA, 149 bp, synthetic construct
  misc_feature 1..149: note = TC15P3_pool2_122
  source 1..149: mol_type = other DNA
gagatcatgtcctaaccctgatcctcttgtcccacagatatccagaaccctctaagaagttcctgcttttttttatttccgagaatgtggttagagacatgaccctgccgtgtaccagctgagagactctaaatccagtgacaagtctg

SEQ ID NO: 852 - DNA, 149 bp, synthetic construct
  misc_feature 1..149: note = TC15P3_pool2_123
  source 1..149: mol_type = other DNA
gagatcatgtcctaaccctgatcctcttgtcccacagatatccagaaccctctaagaagttcctgcttattaaattttcctagaatgtggttagagacatgaccctgccgtgtaccagctgagagactctaaatccagtgacaagtctg

SEQ ID NO: 853 - DNA, 149 bp, synthetic construct
  misc_feature 1..149: note = TC15P3_pool2_124
  source 1..149: mol_type = other DNA
gagatcatgtcctaaccctgatcctcttgtcccacagatatccagaaccctctaagaagttcctgctattaattatttcgaagaatgtggttagagacatgaccctgccgtgtaccagctgagagactctaaatccagtgacaagtctg

SEQ ID NO: 854 - DNA, 149 bp, synthetic construct
  misc_feature 1..149: note = TC15P3_pool2_125
  source 1..149: mol_type = other DNA
gagatcatgtcctaaccctgatcctcttgtcccacagatatccagaaccctctaagaagttcctgctaaatattatttcgcagaatgtggttagagacatgaccctgccgtgtaccagctgagagactctaaatccagtgacaagtctg

SEQ ID NO: 855 - DNA, 149 bp, synthetic construct
  misc_feature 1..149: note = TC15P3_pool3_1
  source 1..149: mol_type = other DNA
gagatcatgtcctaaccctgatcctcttgtcccacagatatccagaaccctctaagaagttcctgcttttaaaattttcggagaatgtggttagagacatgaccctgccgtgtaccagctgagagactctaaatccagtgacaagtctg

SEQ ID NO: 856 - DNA, 149 bp, synthetic construct
  misc_feature 1..149: note = TC15P3_pool3_2
  source 1..149: mol_type = other DNA
gagatcatgtcctaaccctgatcctcttgtcccacagatatccagaaccctctaagaagttcctgctattttaattttcgttgaatgtggttagagacatgaccctgccgtgtaccagctgagagactctaaatccagtgacaagtctg

SEQ ID NO: 857 - DNA, 149 bp, synthetic construct
  misc_feature 1..149: note = TC15P3_pool3_3
  source 1..149: mol_type = other DNA
gagatcatgtcctaaccctgatcctcttgtcccacagatatccagaaccctctaagaagttcctgctataaataatttctaagaatgtggttagagacatgaccctgccgtgtaccagctgagagactctaaatccagtgacaagtctg

SEQ ID NO: 858 - DNA, 149 bp, synthetic construct
  misc_feature 1..149: note = TC15P3_pool3_4
  source 1..149: mol_type = other DNA
gagatcatgtcctaaccctgatcctcttgtcccacagatatccagaaccctctaagaagttcctgctttatattttttctcagaatgtggttagagacatgaccctgccgtgtaccagctgagagactctaaatccagtgacaagtctg

SEQ ID NO: 859 - DNA, 149 bp, synthetic construct
  misc_feature 1..149: note = TC15P3_pool3_5
  source 1..149: mol_type = other DNA
gagatcatgtcctaaccctgatcctcttgtcccacagatatccagaaccctctaagaagttcctgctaatatatttttctgagaatgtggttagagacatgaccctgccgtgtaccagctgagagactctaaatccagtgacaagtctg

SEQ ID NO: 860 - DNA, 149 bp, synthetic construct
  misc_feature 1..149: note = TC15P3_pool3_6
  source 1..149: mol_type = other DNA
gagatcatgtcctaaccctgatcctcttgtcccacagatatccagaaccctctaagaagttcctgcttttaaatatttcttagaatgtggttagagacatgaccctgccgtgtaccagctgagagactctaaatccagtgacaagtctg

SEQ ID NO: 861 - DNA, 147 bp, synthetic construct
  misc_feature 1..147: note = TC13P1_pool1_1
  source 1..147: mol_type = other DNA
gagatcatgtcctaaccctgatcctcttgtcccacagatatccagaaccctctaagaagttcctgctttaataaatcaaagaatgtggttagagacatgaccctgccgtgtaccagctgagagactctaaatccagtgacaagtctg

SEQ ID NO: 862 - DNA, 147 bp, synthetic construct
  misc_feature 1..147: note = TC13P1_pool1_2
  source 1..147: mol_type = other DNA
gagatcatgtcctaaccctgatcctcttgtcccacagatatccagaaccctctaagaagttcctgcttaatttaatcactgaatgtggttagagacatgaccctgccgtgtaccagctgagagactctaaatccagtgacaagtctg

SEQ ID NO: 863 - DNA, 147 bp, synthetic construct
  misc_feature 1..147: note = TC13P1_pool1_3
  source 1..147: mol_type = other DNA
gagatcatgtcctaaccctgatcctcttgtcccacagatatccagaaccctctaagaagttcctgctaattttaatcagagaatgtggttagagacatgaccctgccgtgtaccagctgagagactctaaatccagtgacaagtctg

SEQ ID NO: 864 - DNA, 147 bp, synthetic construct
  misc_feature 1..147: note = TC13P1_pool1_4
  source 1..147: mol_type = other DNA
gagatcatgtcctaaccctgatcctcttgtcccacagatatccagaaccctctaagaagttcctgctttatataatcatagaatgtggttagagacatgaccctgccgtgtaccagctgagagactctaaatccagtgacaagtctg

SEQ ID NO: 865 - DNA, 147 bp, synthetic construct
  misc_feature 1..147: note = TC13P1_pool1_5
  source 1..147: mol_type = other DNA
gagatcatgtcctaaccctgatcctcttgtcccacagatatccagaaccctctaagaagttcctgctaattataatccatgaatgtggttagagacatgaccctgccgtgtaccagctgagagactctaaatccagtgacaagtctg

SEQ ID NO: 866 - DNA, 147 bp, synthetic construct
  misc_feature 1..147: note = TC13P1_pool1_6
  source 1..147: mol_type = other DNA
gagatcatgtcctaaccctgatcctcttgtcccacagatatccagaaccctctaagaagttcctgctttttaaaatcccagaatgtggttagagacatgaccctgccgtgtaccagctgagagactctaaatccagtgacaagtctg

SEQ ID NO: 867 - DNA, 147 bp, synthetic construct
  misc_feature 1..147: note = TC13P1_pool1_7
  source 1..147: mol_type = other DNA
gagatcatgtcctaaccctgatcctcttgtcccacagatatccagaaccctctaagaagttcctgctatttataatccgagaatgtggttagagacatgaccctgccgtgtaccagctgagagactctaaatccagtgacaagtctg

SEQ ID NO: 868 - DNA, 147 bp, synthetic construct
  misc_feature 1..147: note = TC13P1_pool1_8
  source 1..147: mol_type = other DNA
gagatcatgtcctaaccctgatcctcttgtcccacagatatccagaaccctctaagaagttcctgctaaattaaatccttgaatgtggttagagacatgaccctgccgtgtaccagctgagagactctaaatccagtgacaagtctg

SEQ ID NO: 869 - DNA, 147 bp, synthetic construct
  misc_feature 1..147: note = TC13P1_pool1_9
  source 1..147: mol_type = other DNA
gagatcatgtcctaaccctgatcctcttgtcccacagatatccagaaccctctaagaagttcctgctataaaaaatcgaagaatgtggttagagacatgaccctgccgtgtaccagctgagagactctaaatccagtgacaagtctg

SEQ ID NO: 870 - DNA, 147 bp, synthetic construct
  misc_feature 1..147: note = TC13P1_pool1_10
  source 1..147: mol_type = other DNA
gagatcatgtcctaaccctgatcctcttgtcccacagatatccagaaccctctaagaagttcctgcttataataatcgctgaatgtggttagagacatgaccctgccgtgtaccagctgagagactctaaatccagtgacaagtctg

SEQ ID NO: 871 - DNA, 147 bp, synthetic construct
  misc_feature 1..147: note = TC13P1_pool1_11
  source 1..147: mol_type = other DNA
gagatcatgtcctaaccctgatcctcttgtcccacagatatccagaaccctctaagaagttcctgctaaaaataatcggtgaatgtggttagagacatgaccctgccgtgtaccagctgagagactctaaatccagtgacaagtctg

SEQ ID NO: 872 - DNA, 147 bp, synthetic construct
  misc_feature 1..147: note = TC13P1_pool1_12
  source 1..147: mol_type = other DNA
gagatcatgtcctaaccctgatcctcttgtcccacagatatccagaaccctctaagaagttcctgcttttattaatcgtagaatgtggttagagacatgaccctgccgtgtaccagctgagagactctaaatccagtgacaagtctg

SEQ ID NO: 873 - DNA, 147 bp, synthetic construct
  misc_feature 1..147: note = TC13P1_pool1_13
  source 1..147: mol_type = other DNA
gagatcatgtcctaaccctgatcctcttgtcccacagatatccagaaccctctaagaagttcctgctaataaaaatctatgaatgtggttagagacatgaccctgccgtgtaccagctgagagactctaaatccagtgacaagtctg

SEQ ID NO: 874 - DNA, 147 bp, synthetic construct
  misc_feature 1..147: note = TC13P1_pool1_14
  source 1..147: mol_type = other DNA
gagatcatgtcctaaccctgatcctcttgtcccacagatatccagaaccctctaagaagttcctgcttatttaaatctcagaatgtggttagagacatgaccctgccgtgtaccagctgagagactctaaatccagtgacaagtctg

SEQ ID NO: 875 - DNA, 147 bp, synthetic construct
  misc_feature 1..147: note = TC13P1_pool1_15
  source 1..147: mol_type = other DNA
gagatcatgtcctaaccctgatcctcttgtcccacagatatccagaaccctctaagaagttcctgcttattaaaatctgtgaatgtggttagagacatgaccctgccgtgtaccagctgagagactctaaatccagtgacaagtctg

SEQ ID NO: 876 - DNA, 147 bp, synthetic construct
  misc_feature 1..147: note = TC13P1_pool1_16
  source 1..147: mol_type = other DNA
gagatcatgtcctaaccctgatcctcttgtcccacagatatccagaaccctctaagaagttcctgcttttataaatctttgaatgtggttagagacatgaccctgccgtgtaccagctgagagactctaaatccagtgacaagtctg

SEQ ID NO: 877 - DNA, 147 bp, synthetic construct
  misc_feature 1..147: note = TC13P1_pool1_17
  source 1..147: mol_type = other DNA
gagatcatgtcctaaccctgatcctcttgtcccacagatatccagaaccctctaagaagttcctgcttattatactcaaagaatgtggttagagacatgaccctgccgtgtaccagctgagagactctaaatccagtgacaagtctg

SEQ ID NO: 878 - DNA, 147 bp, synthetic construct
  misc_feature 1..147: note = TC13P1_pool1_18
  source 1..147: mol_type = other DNA
gagatcatgtcctaaccctgatcctcttgtcccacagatatccagaaccctctaagaagttcctgctatatatactcactgaatgtggttagagacatgaccctgccgtgtaccagctgagagactctaaatccagtgacaagtctg

SEQ ID NO: 879 - DNA, 147 bp, synthetic construct
  misc_feature 1..147: note = TC13P1_pool1_19
  source 1..147: mol_type = other DNA
gagatcatgtcctaaccctgatcctcttgtcccacagatatccagaaccctctaagaagttcctgctatatttactcagagaatgtggttagagacatgaccctgccgtgtaccagctgagagactctaaatccagtgacaagtctg

SEQ ID NO: 880 - DNA, 147 bp, synthetic construct
  misc_feature 1..147: note = TC13P1_pool1_20
  source 1..147: mol_type = other DNA
gagatcatgtcctaaccctgatcctcttgtcccacagatatccagaaccctctaagaagttcctgctattataactcatagaatgtggttagagacatgaccctgccgtgtaccagctgagagactctaaatccagtgacaagtctg

SEQ ID NO: 881 - DNA, 147 bp, synthetic construct
  misc_feature 1..147: note = TC13P1_pool1_21
  source 1..147: mol_type = other DNA
gagatcatgtcctaaccctgatcctcttgtcccacagatatccagaaccctctaagaagttcctgctaataatactccaagaatgtggttagagacatgaccctgccgtgtaccagctgagagactctaaatccagtgacaagtctg

SEQ ID NO: 882 - DNA, 147 bp, synthetic construct
  misc_feature 1..147: note = TC13P1_pool1_22
  source 1..147: mol_type = other DNA
gagatcatgtcctaaccctgatcctcttgtcccacagatatccagaaccctctaagaagttcctgctataattactccctgaatgtggttagagacatgaccctgccgtgtaccagctgagagactctaaatccagtgacaagtctg

SEQ ID NO: 883 - DNA, 147 bp, synthetic construct
  misc_feature 1..147: note = TC13P1_pool1_23
  source 1..147: mol_type = other DNA
gagatcatgtcctaaccctgatcctcttgtcccacagatatccagaaccctctaagaagttcctgctaaatatactccgagaatgtggttagagacatgaccctgccgtgtaccagctgagagactctaaatccagtgacaagtctg

SEQ ID NO: 884 - DNA, 147 bp, synthetic construct
  misc_feature 1..147: note = TC13P1_pool1_24
  source 1..147: mol_type = other DNA
gagatcatgtcctaaccctgatcctcttgtcccacagatatccagaaccctctaagaagttcctgctaaaataactcctagaatgtggttagagacatgaccctgccgtgtaccagctgagagactctaaatccagtgacaagtctg

SEQ ID NO: 885 - DNA, 147 bp, synthetic construct
  misc_feature 1..147: note = TC13P1_pool1_25
  source 1..147: mol_type = other DNA
gagatcatgtcctaaccctgatcctcttgtcccacagatatccagaaccctctaagaagttcctgctattttaactcgatgaatgtggttagagacatgaccctgccgtgtaccagctgagagactctaaatccagtgacaagtctg

SEQ ID NO: 886 - DNA, 147 bp, synthetic construct
  misc_feature 1..147: note = TC13P1_pool1_26
  source 1..147: mol_type = other DNA
gagatcatgtcctaaccctgatcctcttgtcccacagatatccagaaccctctaagaagttcctgctttttatactcgctgaatgtggttagagacatgaccctgccgtgtaccagctgagagactctaaatccagtgacaagtctg

SEQ ID NO: 887 - DNA, 147 bp, synthetic construct
  misc_feature 1..147: note = TC13P1_pool1_27
  source 1..147: mol_type = other DNA
gagatcatgtcctaaccctgatcctcttgtcccacagatatccagaaccctctaagaagttcctgcttaaaatactcggagaatgtggttagagacatgaccctgccgtgtaccagctgagagactctaaatccagtgacaagtctg

SEQ ID NO: 888 - DNA, 147 bp, synthetic construct
  misc_feature 1..147: note = TC13P1_pool1_28
  source 1..147: mol_type = other DNA
gagatcatgtcctaaccctgatcctcttgtcccacagatatccagaaccctctaagaagttcctgctattaatactcgttgaatgtggttagagacatgaccctgccgtgtaccagctgagagactctaaatccagtgacaagtctg

SEQ ID NO: 889 - DNA, 147 bp, synthetic construct
  misc_feature 1..147: note = TC13P1_pool1_29
  source 1..147: mol_type = other DNA
gagatcatgtcctaaccctgatcctcttgtcccacagatatccagaaccctctaagaagttcctgcttaataaactctaagaatgtggttagagacatgaccctgccgtgtaccagctgagagactctaaatccagtgacaagtctg

SEQ ID NO: 890 - DNA, 147 bp, synthetic construct
  misc_feature 1..147: note = TC13P1_pool1_30
  source 1..147: mol_type = other DNA
gagatcatgtcctaaccctgatcctcttgtcccacagatatccagaaccctctaagaagttcctgcttaaataactctctgaatgtggttagagacatgaccctgccgtgtaccagctgagagactctaaatccagtgacaagtctg

SEQ ID NO: 891 - DNA, 147 bp, synthetic construct
  misc_feature 1..147: note = TC13P1_pool1_31
  source 1..147: mol_type = other DNA
gagatcatgtcctaaccctgatcctcttgtcccacagatatccagaaccctctaagaagttcctgcttataaaactctgagaatgtggttagagacatgaccctgccgtgtaccagctgagagactctaaatccagtgacaagtctg

SEQ ID NO: 892 - DNA, 147 bp, synthetic construct
  misc_feature 1..147: note = TC13P1_pool1_32
  source 1..147: mol_type = other DNA
gagatcatgtcctaaccctgatcctcttgtcccacagatatccagaaccctctaagaagttcctgctttaaatactctttgaatgtggttagagacatgaccctgccgtgtaccagctgagagactctaaatccagtgacaagtctg

SEQ ID NO: 893 - DNA, 147 bp, synthetic construct
  misc_feature 1..147: note = TC13P1_pool1_33
  source 1..147: mol_type = other DNA
gagatcatgtcctaaccctgatcctcttgtcccacagatatccagaaccctctaagaagttcctgctaatattagtcaatgaatgtggttagagacatgaccctgccgtgtaccagctgagagactctaaatccagtgacaagtctg

SEQ ID NO: 894 - DNA, 147 bp, synthetic construct
  misc_feature 1..147: note = TC13P1_pool1_34
  source 1..147: mol_type = other DNA
gagatcatgtcctaaccctgatcctcttgtcccacagatatccagaaccctctaagaagttcctgctttataaagtcactgaatgtggttagagacatgaccctgccgtgtaccagctgagagactctaaatccagtgacaagtctg

SEQ ID NO: 895 - DNA, 147 bp, synthetic construct
  misc_feature 1..147: note = TC13P1_pool2_1
  source 1..147: mol_type = other DNA
gagatcatgtcctaaccctgatcctcttgtcccacagatatccagaaccctctaagaagttcctgctttaataagtcagagaatgtggttagagacatgaccctgccgtgtaccagctgagagactctaaatccagtgacaagtctg

SEQ ID NO: 896 - DNA, 147 bp, synthetic construct
  misc_feature 1..147: note = TC13P1_pool2_2
  source 1..147: mol_type = other DNA
gagatcatgtcctaaccctgatcctcttgtcccacagatatccagaaccctctaagaagttcctgcttaatttagtcattgaatgtggttagagacatgaccctgccgtgtaccagctgagagactctaaatccagtgacaagtctg

SEQ ID NO: 897 - DNA, 147 bp, synthetic construct
  misc_feature 1..147: note = TC13P1_pool2_3
  source 1..147: mol_type = other DNA
gagatcatgtcctaaccctgatcctcttgtcccacagatatccagaaccctctaagaagttcctgctaattttagtccaagaatgtggttagagacatgaccctgccgtgtaccagctgagagactctaaatccagtgacaagtctg

SEQ ID NO: 898 - DNA, 147 bp, synthetic construct
  misc_feature 1..147: note = TC13P1_pool2_4
  source 1..147: mol_type = other DNA
gagatcatgtcctaaccctgatcctcttgtcccacagatatccagaaccctctaagaagttcctgctttatatagtcccagaatgtggttagagacatgaccctgccgtgtaccagctgagagactctaaatccagtgacaagtctg

SEQ ID NO: 899 - DNA, 147 bp, synthetic construct
  misc_feature 1..147: note = TC13P1_pool2_5
  source 1..147: mol_type = other DNA
gagatcatgtcctaaccctgatcctcttgtcccacagatatccagaaccctctaagaagttcctgctaattatagtccgtgaatgtggttagagacatgaccctgccgtgtaccagctgagagactctaaatccagtgacaagtctg

SEQ ID NO: 900 - DNA, 147 bp, synthetic construct
  misc_feature 1..147: note = TC13P1_pool2_6
  source 1..147: mol_type = other DNA
gagatcatgtcctaaccctgatcctcttgtcccacagatatccagaaccctctaagaagttcctgctttttaaagtcctagaatgtggttagagacatgaccctgccgtgtaccagctgagagactctaaatccagtgacaagtctg

SEQ ID NO: 901 - DNA, 147 bp, synthetic construct
  misc_feature 1..147: note = TC13P1_pool2_7
  source 1..147: mol_type = other DNA
gagatcatgtcctaaccctgatcctcttgtcccacagatatccagaaccctctaagaagttcctgctatttatagtcgaagaatgtggttagagacatgaccctgccgtgtaccagctgagagactctaaatccagtgacaagtctg

SEQ ID NO: 902 - DNA, 147 bp, synthetic construct
  misc_feature 1..147: note = TC13P1_pool2_8
  source 1..147: mol_type = other DNA
gagatcatgtcctaaccctgatcctcttgtcccacagatatccagaaccctctaagaagttcctgctaaattaagtcgctgaatgtggttagagacatgaccctgccgtgtaccagctgagagactctaaatccagtgacaagtctg

SEQ ID NO: 903 - DNA, 147 bp, synthetic construct
  misc_feature 1..147: note = TC13P1_pool2_9
  source 1..147: mol_type = other DNA
gagatcatgtcctaaccctgatcctcttgtcccacagatatccagaaccctctaagaagttcctgctataaaaagtcggagaatgtggttagagacatgaccctgccgtgtaccagctgagagactctaaatccagtgacaagtctg

SEQ ID NO: 904 - DNA, 147 bp, synthetic construct
  misc_feature 1..147: note = TC13P1_pool2_10
  source 1..147: mol_type = other DNA
gagatcatgtcctaaccctgatcctcttgtcccacagatatccagaaccctctaagaagttcctgcttataatagtcgttgaatgtggttagagacatgaccctgccgtgtaccagctgagagactctaaatccagtgacaagtctg

SEQ ID NO: 905 - DNA, 147 bp, synthetic construct
  misc_feature 1..147: note = TC13P1_pool2_11
  source 1..147: mol_type = other DNA
gagatcatgtcctaaccctgatcctcttgtcccacagatatccagaaccctctaagaagttcctgctaaaaatagtctatgaatgtggttagagacatgaccctgccgtgtaccagctgagagactctaaatccagtgacaagtctg

SEQ ID NO: 906 - DNA, 147 bp, synthetic construct
  misc_feature 1..147: note = TC13P1_pool2_12
  source 1..147: mol_type = other DNA
gagatcatgtcctaaccctgatcctcttgtcccacagatatccagaaccctctaagaagttcctgcttttattagtctcagaatgtggttagagacatgaccctgccgtgtaccagctgagagactctaaatccagtgacaagtctg

SEQ ID NO: 907 - DNA, 147 bp, synthetic construct
  misc_feature 1..147: note = TC13P1_pool2_13
  source 1..147: mol_type = other DNA
gagatcatgtcctaaccctgatcctcttgtcccacagatatccagaaccctctaagaagttcctgctaataaaagtctgtgaatgtggttagagacatgaccctgccgtgtaccagctgagagactctaaatccagtgacaagtctg

SEQ ID NO: 908 - DNA, 147 bp, synthetic construct
  misc_feature 1..147: note = TC13P1_pool2_14
  source 1..147: mol_type = other DNA
gagatcatgtcctaaccctgatcctcttgtcccacagatatccagaaccctctaagaagttcctgcttatttaagtcttagaatgtggttagagacatgaccctgccgtgtaccagctgagagactctaaatccagtgacaagtctg

SEQ ID NO: 909 - DNA, 147 bp, synthetic construct
  misc_feature 1..147: note = TC13P1_pool2_15
  source 1..147: mol_type = other DNA
gagatcatgtcctaaccctgatcctcttgtcccacagatatccagaaccctctaagaagttcctgcttattaaattcaatgaatgtggttagagacatgaccctgccgtgtaccagctgagagactctaaatccagtgacaagtctg

SEQ ID NO: 910 - DNA, 147 bp, synthetic construct
  misc_feature 1..147: note = TC13P1_pool2_16
  source 1..147: mol_type = other DNA
gagatcatgtcctaaccctgatcctcttgtcccacagatatccagaaccctctaagaagttcctgcttttataattcactgaatgtggttagagacatgaccctgccgtgtaccagctgagagactctaaatccagtgacaagtctg

SEQ ID NO: 911 - DNA, 147 bp, synthetic construct
  misc_feature 1..147: note = TC13P1_pool2_17
  source 1..147: mol_type = other DNA
gagatcatgtcctaaccctgatcctcttgtcccacagatatccagaaccctctaagaagttcctgcttattatattcagagaatgtggttagagacatgaccctgccgtgtaccagctgagagactctaaatccagtgacaagtctg

SEQ ID NO: 912 - DNA, 147 bp, synthetic construct
  misc_feature 1..147: note = TC13P1_pool2_18
  source 1..147: mol_type = other DNA
gagatcatgtcctaaccctgatcctcttgtcccacagatatccagaaccctctaagaagttcctgctatatatattcattgaatgtggttagagacatgaccctgccgtgtaccagctgagagactctaaatccagtgacaagtctg

SEQ ID NO: 913 - DNA, 147 bp, synthetic construct
  misc_feature 1..147: note = TC13P1_pool2_19
  source 1..147: mol_type = other DNA
gagatcatgtcctaaccctgatcctcttgtcccacagatatccagaaccctctaagaagttcctgctatatttattccaagaatgtggttagagacatgaccctgccgtgtaccagctgagagactctaaatccagtgacaagtctg

SEQ ID NO: 914 - DNA, 147 bp, synthetic construct
  misc_feature 1..147: note = TC13P1_pool2_20
  source 1..147: mol_type = other DNA
gagatcatgtcctaaccctgatcctcttgtcccacagatatccagaaccctctaagaagttcctgctattataattcccagaatgtggttagagacatgaccctgccgtgtaccagctgagagactctaaatccagtgacaagtctg

SEQ ID NO: 915 - DNA, 147 bp, synthetic construct
  misc_feature 1..147: note = TC13P1_pool2_21
  source 1..147: mol_type = other DNA
gagatcatgtcctaaccctgatcctcttgtcccacagatatccagaaccctctaagaagttcctgctaataatattccgagaatgtggttagagacatgaccctgccgtgtaccagctgagagactctaaatccagtgacaagtctg

SEQ ID NO: 916 - DNA, 147 bp, synthetic construct
  misc_feature 1..147: note = TC13P1_pool2_22
  source 1..147: mol_type = other DNA
gagatcatgtcctaaccctgatcctcttgtcccacagatatccagaaccctctaagaagttcctgctataattattccttgaatgtggttagagacatgaccctgccgtgtaccagctgagagactctaaatccagtgacaagtctg

SEQ ID NO: 917 - DNA, 147 bp, synthetic construct
  misc_feature 1..147: note = TC13P1_pool2_23
  source 1..147: mol_type = other DNA
gagatcatgtcctaaccctgatcctcttgtcccacagatatccagaaccctctaagaagttcctgctaaatatattcgaagaatgtggttagagacatgaccctgccgtgtaccagctgagagactctaaatccagtgacaagtctg

SEQ ID NO: 918 - DNA, 147 bp, synthetic construct
  misc_feature 1..147: note = TC13P1_pool2_24
  source 1..147: mol_type = other DNA
gagatcatgtcctaaccctgatcctcttgtcccacagatatccagaaccctctaagaagttcctgctaaaataattcgcagaatgtggttagagacatgaccctgccgtgtaccagctgagagactctaaatccagtgacaagtctg

SEQ ID NO: 919 - DNA, 147 bp, synthetic construct
  misc_feature 1..147: note = TC13P1_pool2_25
  source 1..147: mol_type = other DNA
gagatcatgtcctaaccctgatcctcttgtcccacagatatccagaaccctctaagaagttcctgctattttaattcggtgaatgtggttagagacatgaccctgccgtgtaccagctgagagactctaaatccagtgacaagtctg

SEQ ID NO: 920 - DNA, 147 bp, synthetic construct
  misc_feature 1..147: note = TC13P1_pool2_26
  source 1..147: mol_type = other DNA
gagatcatgtcctaaccctgatcctcttgtcccacagatatccagaaccctctaagaagttcctgctttttatattcgttgaatgtggttagagacatgaccctgccgtgtaccagctgagagactctaaatccagtgacaagtctg

SEQ ID NO: 921 - DNA, 147 bp, synthetic construct
  misc_feature 1..147: note = TC13P1_pool2_27
  source 1..147: mol_type = other DNA
gagatcatgtcctaaccctgatcctcttgtcccacagatatccagaaccctctaagaagttcctgcttaaaatattctaagaatgtggttagagacatgaccctgccgtgtaccagctgagagactctaaatccagtgacaagtctg

SEQ ID NO: 922 - DNA, 147 bp, synthetic construct
  misc_feature 1..147: note = TC13P1_pool2_28
  source 1..147: mol_type = other DNA
gagatcatgtcctaaccctgatcctcttgtcccacagatatccagaaccctctaagaagttcctgctattaatattctctgaatgtggttagagacatgaccctgccgtgtaccagctgagagactctaaatccagtgacaagtctg

SEQ ID NO: 923 - DNA, 147 bp, synthetic construct
  misc_feature 1..147: note = TC13P1_pool2_29
  source 1..147: mol_type = other DNA
gagatcatgtcctaaccctgatcctcttgtcccacagatatccagaaccctctaagaagttcctgcttaataaattctgagaatgtggttagagacatgaccctgccgtgtaccagctgagagactctaaatccagtgacaagtctg

SEQ ID NO: 924 - DNA, 147 bp, synthetic construct
  misc_feature 1..147: note = TC13P1_pool2_30
  source 1..147: mol_type = other DNA
gagatcatgtcctaaccctgatcctcttgtcccacagatatccagaaccctctaagaagttcctgcttaaataattctttgaatgtggttagagacatgaccctgccgtgtaccagctgagagactctaaatccagtgacaagtctg

SEQ ID NO: 925 - DNA, 147 bp, synthetic construct
  misc_feature 1..147: note = TC13P1_pool2_31
  source 1..147: mol_type = other DNA
gagatcatgtcctaaccctgatcctcttgtcccacagatatccagaaccctctaagaagttcctgcttataaacatcaaagaatgtggttagagacatgaccctgccgtgtaccagctgagagactctaaatccagtgacaagtctg

SEQ ID NO: 926 - DNA, 147 bp, synthetic construct
  misc_feature 1..147: note = TC13P1_pool2_32
  source 1..147: mol_type = other DNA
gagatcatgtcctaaccctgatcctcttgtcccacagatatccagaaccctctaagaagttcctgctttaaatcatcactgaatgtggttagagacatgaccctgccgtgtaccagctgagagactctaaatccagtgacaagtctg

SEQ ID NO: 927 - DNA, 147 bp, synthetic construct
  misc_feature 1..147: note = TC13P1_pool2_33
  source 1..147: mol_type = other DNA
gagatcatgtcctaaccctgatcctcttgtcccacagatatccagaaccctctaagaagttcctgctaatattcatcagtgaatgtggttagagacatgaccctgccgtgtaccagctgagagactctaaatccagtgacaagtctg

SEQ ID NO: 928 - DNA, 147 bp, synthetic construct
  misc_feature 1..147: note = TC13P1_pool2_34
  source 1..147: mol_type = other DNA
gagatcatgtcctaaccctgatcctcttgtcccacagatatccagaaccctctaagaagttcctgctttataacatcattgaatgtggttagagacatgaccctgccgtgtaccagctgagagactctaaatccagtgacaagtctg

SEQ ID NO: 929 - DNA, 147 bp, synthetic construct
  misc_feature 1..147: note = TC13P2_pool3_1
  source 1..147: mol_type = other DNA
gagatcatgtcctaaccctgatcctcttgtcccacagatatccagaaccctctaagaagttcctgctttaatacatccaagaatgtggttagagacatgaccctgccgtgtaccagctgagagactctaaatccagtgacaagtctg

SEQ ID NO: 930 - DNA, 147 bp, synthetic construct
  misc_feature 1..147: note = TC13P2_pool3_2
  source 1..147: mol_type = other DNA
gagatcatgtcctaaccctgatcctcttgtcccacagatatccagaaccctctaagaagttcctgcttaatttcatccctgaatgtggttagagacatgaccctgccgtgtaccagctgagagactctaaatccagtgacaagtctg

SEQ ID NO: 931 - DNA, 147 bp, synthetic construct
  misc_feature 1..147: note = TC13P2_pool3_3
  source 1..147: mol_type = other DNA
gagatcatgtcctaaccctgatcctcttgtcccacagatatccagaaccctctaagaagttcctgctaattttcatccgagaatgtggttagagacatgaccctgccgtgtaccagctgagagactctaaatccagtgacaagtctg

SEQ ID NO: 932 - DNA, 147 bp, synthetic construct
  misc_feature 1..147: note = TC13P2_pool3_4
  source 1..147: mol_type = other DNA
gagatcatgtcctaaccctgatcctcttgtcccacagatatccagaaccctctaagaagttcctgctttatatcatcctagaatgtggttagagacatgaccctgccgtgtaccagctgagagactctaaatccagtgacaagtctg

SEQ ID NO: 933 - DNA, 147 bp, synthetic construct
  misc_feature 1..147: note = TC13P2_pool3_5
  source 1..147: mol_type = other DNA
gagatcatgtcctaaccctgatcctcttgtcccacagatatccagaaccctctaagaagttcctgctaattatcatcgatgaatgtggttagagacatgaccctgccgtgtaccagctgagagactctaaatccagtgacaagtctg

SEQ ID NO: 934 - DNA, 147 bp, synthetic construct
  misc_feature 1..147: note = TC13P2_pool3_6
  source 1..147: mol_type = other DNA
gagatcatgtcctaaccctgatcctcttgtcccacagatatccagaaccctctaagaagttcctgctttttaacatcgcagaatgtggttagagacatgaccctgccgtgtaccagctgagagactctaaatccagtgacaagtctg

SEQ ID NO: 935 - DNA, 147 bp, synthetic construct
  misc_feature 1..147: note = TC13P2_pool3_7
  source 1..147: mol_type = other DNA
gagatcatgtcctaaccctgatcctcttgtcccacagatatccagaaccctctaagaagttcctgctatttatcatcggagaatgtggttagagacatgaccctgccgtgtaccagctgagagactctaaatccagtgacaagtctg

SEQ ID NO: 936 - DNA, 147 bp, synthetic construct
  misc_feature 1..147: note = TC13P2_pool3_8
  source 1..147: mol_type = other DNA
gagatcatgtcctaaccctgatcctcttgtcccacagatatccagaaccctctaagaagttcctgctaaattacatcgttgaatgtggttagagacatgaccctgccgtgtaccagctgagagactctaaatccagtgacaagtctg

SEQ ID NO: 937 - DNA, 147 bp, synthetic construct
  misc_feature 1..147: note = TC13P2_pool3_9
  source 1..147: mol_type = other DNA
gagatcatgtcctaaccctgatcctcttgtcccacagatatccagaaccctctaagaagttcctgctataaaacatctaagaatgtggttagagacatgaccctgccgtgtaccagctgagagactctaaatccagtgacaagtctg

SEQ ID NO: 938 - DNA, 147 bp, synthetic construct
  misc_feature 1..147: note = TC13P2_pool3_10
  source 1..147: mol_type = other DNA
gagatcatgtcctaaccctgatcctcttgtcccacagatatccagaaccctctaagaagttcctgcttataatcatctctgaatgtggttagagacatgaccctgccgtgtaccagctgagagactctaaatccagtgacaagtctg

SEQ ID NO: 939 - DNA, 147 bp, synthetic construct
  misc_feature 1..147: note = TC13P2_pool3_11
  source 1..147: mol_type = other DNA
gagatcatgtcctaaccctgatcctcttgtcccacagatatccagaaccctctaagaagttcctgctaaaaatcatctgtgaatgtggttagagacatgaccctgccgtgtaccagctgagagactctaaatccagtgacaagtctg

SEQ ID NO: 940 - DNA, 147 bp, synthetic construct
  misc_feature 1..147: note = TC13P2_pool3_12
  source 1..147: mol_type = other DNA
gagatcatgtcctaaccctgatcctcttgtcccacagatatccagaaccctctaagaagttcctgcttttattcatcttagaatgtggttagagacatgaccctgccgtgtaccagctgagagactctaaatccagtgacaagtctg

SEQ ID NO: 941 - DNA, 147 bp, synthetic construct
  misc_feature 1..147: note = TC13P2_pool3_13
  source 1..147: mol_type = other DNA
gagatcatgtcctaaccctgatcctcttgtcccacagatatccagaaccctctaagaagttcctgctaataaacctcaatgaatgtggttagagacatgaccctgccgtgtaccagctgagagactctaaatccagtgacaagtctg

SEQ ID NO: 942 - DNA, 147 bp, synthetic construct
  misc_feature 1..147: note = TC13P2_pool3_14
  source 1..147: mol_type = other DNA
gagatcatgtcctaaccctgatcctcttgtcccacagatatccagaaccctctaagaagttcctgcttatttacctcacagaatgtggttagagacatgaccctgccgtgtaccagctgagagactctaaatccagtgacaagtctg

SEQ ID NO: 943 - DNA, 147 bp, synthetic construct
  misc_feature 1..147: note = TC13P2_pool3_15
  source 1..147: mol_type = other DNA
gagatcatgtcctaaccctgatcctcttgtcccacagatatccagaaccctctaagaagttcctgcttattaacctcagtgaatgtggttagagacatgaccctgccgtgtaccagctgagagactctaaatccagtgacaagtctg

SEQ ID NO: 944 - DNA, 147 bp, synthetic construct
  misc_feature 1..147: note = TC13P2_pool3_16
  source 1..147: mol_type = other DNA
gagatcatgtcctaaccctgatcctcttgtcccacagatatccagaaccctctaagaagttcctgcttttatacctcattgaatgtggttagagacatgaccctgccgtgtaccagctgagagactctaaatccagtgacaagtctg

SEQ ID NO: 945 - DNA, 147 bp, synthetic construct
  misc_feature 1..147: note = TC13P2_pool3_17
  source 1..147: mol_type = other DNA
gagatcatgtcctaaccctgatcctcttgtcccacagatatccagaaccctctaagaagttcctgcttattatcctccaagaatgtggttagagacatgaccctgccgtgtaccagctgagagactctaaatccagtgacaagtctg

SEQ ID NO: 946 - DNA, 147 bp, synthetic construct
  misc_feature 1..147: note = TC13P2_pool3_18
  source 1..147: mol_type = other DNA
gagatcatgtcctaaccctgatcctcttgtcccacagatatccagaaccctctaagaagttcctgctatatatcctccctgaatgtggttagagacatgaccctgccgtgtaccagctgagagactctaaatccagtgacaagtctg

SEQ ID NO: 947 - DNA, 147 bp, synthetic construct
  misc_feature 1..147: note = TC13P2_pool3_19
  source 1..147: mol_type = other DNA
gagatcatgtcctaaccctgatcctcttgtcccacagatatccagaaccctctaagaagttcctgctatatttcctccgagaatgtggttagagacatgaccctgccgtgtaccagctgagagactctaaatccagtgacaagtctg

SEQ ID NO: 948 - DNA, 147 bp, synthetic construct
  misc_feature 1..147: note = TC13P2_pool3_20
  source 1..147: mol_type = other DNA
gagatcatgtcctaaccctgatcctcttgtcccacagatatccagaaccctctaagaagttcctgctattatacctcctagaatgtggttagagacatgaccctgccgtgtaccagctgagagactctaaatccagtgacaagtctg

SEQ ID NO: 949 - DNA, 147 bp, synthetic construct
  misc_feature 1..147: note = TC13P2_pool3_21
  source 1..147: mol_type = other DNA
gagatcatgtcctaaccctgatcctcttgtcccacagatatccagaaccctctaagaagttcctgctaataatcctcgaagaatgtggttagagacatgaccctgccgtgtaccagctgagagactctaaatccagtgacaagtctg

SEQ ID NO: 950 - DNA, 147 bp, synthetic construct
  misc_feature 1..147: note = TC13P2_pool3_22
  source 1..147: mol_type = other DNA
gagatcatgtcctaaccctgatcctcttgtcccacagatatccagaaccctctaagaagttcctgctataattcctcgctgaatgtggttagagacatgaccctgccgtgtaccagctgagagactctaaatccagtgacaagtctg

SEQ ID NO: 951 - DNA, 147 bp, synthetic construct
  misc_feature 1..147: note = TC13P2_pool3_23
  source 1..147: mol_type = other DNA
gagatcatgtcctaaccctgatcctcttgtcccacagatatccagaaccctctaagaagttcctgctaaatatcctcggagaatgtggttagagacatgaccctgccgtgtaccagctgagagactctaaatccagtgacaagtctg

SEQ ID NO: 952 - DNA, 147 bp, synthetic construct
  misc_feature 1..147: note = TC13P2_pool3_24
  source 1..147: mol_type = other DNA
gagatcatgtcctaaccctgatcctcttgtcccacagatatccagaaccctctaagaagttcctgctaaaatacctcgtagaatgtggttagagacatgaccctgccgtgtaccagctgagagactctaaatccagtgacaagtctg

SEQ ID NO: 953 - DNA, 147 bp, synthetic construct
  misc_feature 1..147: note = TC13P2_pool3_25
  source 1..147: mol_type = other DNA
gagatcatgtcctaaccctgatcctcttgtcccacagatatccagaaccctctaagaagttcctgctattttacctctatgaatgtggttagagacatgaccctgccgtgtaccagctgagagactctaaatccagtgacaagtctg

SEQ ID NO: 954 - DNA, 147 bp, synthetic construct
  misc_feature 1..147: note = TC13P2_pool3_26
  source 1..147: mol_type = other DNA
gagatcatgtcctaaccctgatcctcttgtcccacagatatccagaaccctctaagaagttcctgctttttatcctctctgaatgtggttagagacatgaccctgccgtgtaccagctgagagactctaaatccagtgacaagtctg

SEQ ID NO: 955 - DNA, 147 bp, synthetic construct
  misc_feature 1..147: note = TC13P2_pool3_27
  source 1..147: mol_type = other DNA
gagatcatgtcctaaccctgatcctcttgtcccacagatatccagaaccctctaagaagttcctgcttaaaatcctctgagaatgtggttagagacatgaccctgccgtgtaccagctgagagactctaaatccagtgacaagtctg

SEQ ID NO: 956 - DNA, 147 bp, synthetic construct
  misc_feature 1..147: note = TC13P2_pool3_28
  source 1..147: mol_type = other DNA
gagatcatgtcctaaccctgatcctcttgtcccacagatatccagaaccctctaagaagttcctgctattaatcctctttgaatgtggttagagacatgaccctgccgtgtaccagctgagagactctaaatccagtgacaagtctg

SEQ ID NO: 957 - DNA, 147 bp, synthetic construct
  misc_feature 1..147: note = TC13P2_pool3_29
  source 1..147: mol_type = other DNA
gagatcatgtcctaaccctgatcctcttgtcccacagatatccagaaccctctaagaagttcctgcttaataacgtcaaagaatgtggttagagacatgaccctgccgtgtaccagctgagagactctaaatccagtgacaagtctg

SEQ ID NO: 958 - DNA, 147 bp, synthetic construct
  misc_feature 1..147: note = TC13P2_pool3_30
  source 1..147: mol_type = other DNA
gagatcatgtcctaaccctgatcctcttgtcccacagatatccagaaccctctaagaagttcctgcttaaatacgtcactgaatgtggttagagacatgaccctgccgtgtaccagctgagagactctaaatccagtgacaagtctg

SEQ ID NO: 959 - DNA, 147 bp, synthetic construct
  misc_feature 1..147: note = TC13P2_pool3_31
  source 1..147: mol_type = other DNA
gagatcatgtcctaaccctgatcctcttgtcccacagatatccagaaccctctaagaagttcctgcttataaacgtcagagaatgtggttagagacatgaccctgccgtgtaccagctgagagactctaaatccagtgacaagtctg

SEQ ID NO: 960 - DNA, 147 bp, synthetic construct
  misc_feature 1..147: note = TC13P2_pool3_32
  source 1..147: mol_type = other DNA
gagatcatgtcctaaccctgatcctcttgtcccacagatatccagaaccctctaagaagttcctgctttaaatcgtcattgaatgtggttagagacatgaccctgccgtgtaccagctgagagactctaaatccagtgacaagtctg

SEQ ID NO: 961 - DNA, 147 bp, synthetic construct
  misc_feature 1..147: note = TC13P2_pool3_33
  source 1..147: mol_type = other DNA
gagatcatgtcctaaccctgatcctcttgtcccacagatatccagaaccctctaagaagttcctgctaatattcgtccatgaatgtggttagagacatgaccctgccgtgtaccagctgagagactctaaatccagtgacaagtctg

SEQ ID NO: 962 - DNA, 147 bp, synthetic construct
  misc_feature 1..147: note = TC13P2_pool3_34
  source 1..147: mol_type = other DNA
gagatcatgtcctaaccctgatcctcttgtcccacagatatccagaaccctctaagaagttcctgctttataacgtccctgaatgtggttagagacatgaccctgccgtgtaccagctgagagactctaaatccagtgacaagtctg

SEQ ID NO: 963 - DNA, 147 bp, synthetic construct
  misc_feature 1..147: note = TC13P2_pool4_1
  source 1..147: mol_type = other DNA
gagatcatgtcctaaccctgatcctcttgtcccacagatatccagaaccctctaagaagttcctgctttaatacgtccgagaatgtggttagagacatgaccctgccgtgtaccagctgagagactctaaatccagtgacaagtctg

SEQ ID NO: 964 - DNA, 147 bp, synthetic construct
  misc_feature 1..147: note = TC13P2_pool4_2
  source 1..147: mol_type = other DNA
gagatcatgtcctaaccctgatcctcttgtcccacagatatccagaaccctctaagaagttcctgcttaatttcgtccttgaatgtggttagagacatgaccctgccgtgtaccagctgagagactctaaatccagtgacaagtctg

SEQ ID NO: 965 - DNA, 147 bp, synthetic construct
  misc_feature 1..147: note = TC13P2_pool4_3
  source 1..147: mol_type = other DNA
gagatcatgtcctaaccctgatcctcttgtcccacagatatccagaaccctctaagaagttcctgctaattttcgtcgaagaatgtggttagagacatgaccctgccgtgtaccagctgagagactctaaatccagtgacaagtctg

SEQ ID NO: 966 - DNA, 147 bp, synthetic construct
  misc_feature 1..147: note = TC13P2_pool4_4
  source 1..147: mol_type = other DNA
gagatcatgtcctaaccctgatcctcttgtcccacagatatccagaaccctctaagaagttcctgctttatatcgtcgcagaatgtggttagagacatgaccctgccgtgtaccagctgagagactctaaatccagtgacaagtctg

SEQ ID NO: 967 - DNA, 147 bp, synthetic construct
  misc_feature 1..147: note = TC13P2_pool4_5
  source 1..147: mol_type = other DNA
gagatcatgtcctaaccctgatcctcttgtcccacagatatccagaaccctctaagaagttcctgctaattatcgtcggtgaatgtggttagagacatgaccctgccgtgtaccagctgagagactctaaatccagtgacaagtctg

SEQ ID NO: 968 - DNA, 147 bp, synthetic construct
  misc_feature 1..147: note = TC13P2_pool4_6
  source 1..147: mol_type = other DNA
gagatcatgtcctaaccctgatcctcttgtcccacagatatccagaaccctctaagaagttcctgctttttaacgtcgtagaatgtggttagagacatgaccctgccgtgtaccagctgagagactctaaatccagtgacaagtctg

SEQ ID NO: 969 - DNA, 147 bp, synthetic construct
  misc_feature 1..147: note = TC13P2_pool4_7
  source 1..147: mol_type = other DNA
gagatcatgtcctaaccctgatcctcttgtcccacagatatccagaaccctctaagaagttcctgctatttatcgtctaagaatgtggttagagacatgaccctgccgtgtaccagctgagagactctaaatccagtgacaagtctg

SEQ ID NO: 970 - DNA, 147 bp, synthetic construct
  misc_feature 1..147: note = TC13P2_pool4_8
  source 1..147: mol_type = other DNA
gagatcatgtcctaaccctgatcctcttgtcccacagatatccagaaccctctaagaagttcctgctaaattacgtctctgaatgtggttagagacatgaccctgccgtgtaccagctgagagactctaaatccagtgacaagtctg

SEQ ID NO: 971 - DNA, 147 bp, synthetic construct
  misc_feature 1..147: note = TC13P2_pool4_9
  source 1..147: mol_type = other DNA
gagatcatgtcctaaccctgatcctcttgtcccacagatatccagaaccctctaagaagttcctgctataaaacgtctgagaatgtggttagagacatgaccctgccgtgtaccagctgagagactctaaatccagtgacaagtctg

SEQ ID NO: 972 - DNA, 147 bp, synthetic construct
  misc_feature 1..147: note = TC13P2_pool4_10
  source 1..147: mol_type = other DNA
gagatcatgtcctaaccctgatcctcttgtcccacagatatccagaaccctctaagaagttcctgcttataatcgtctttgaatgtggttagagacatgaccctgccgtgtaccagctgagagactctaaatccagtgacaagtctg

SEQ ID NO: 973 - DNA, 147 bp, synthetic construct
  misc_feature 1..147: note = TC13P2_pool4_11
  source 1..147: mol_type = other DNA
gagatcatgtcctaaccctgatcctcttgtcccacagatatccagaaccctctaagaagttcctgctaaaaatcttcaatgaatgtggttagagacatgaccctgccgtgtaccagctgagagactctaaatccagtgacaagtctg

SEQ ID NO: 974 - DNA, 147 bp, synthetic construct
  misc_feature 1..147: note = TC13P2_pool4_12
  source 1..147: mol_type = other DNA
gagatcatgtcctaaccctgatcctcttgtcccacagatatccagaaccctctaagaagttcctgcttttattcttcacagaatgtggttagagacatgaccctgccgtgtaccagctgagagactctaaatccagtgacaagtctg

SEQ ID NO: 975 - DNA, 147 bp, synthetic construct
  misc_feature 1..147: note = TC13P2_pool4_13
  source 1..147: mol_type = other DNA
gagatcatgtcctaaccctgatcctcttgtcccacagatatccagaaccctctaagaagttcctgctaataaacttcagtgaatgtggttagagacatgaccctgccgtgtaccagctgagagactctaaatccagtgacaagtctg

SEQ ID NO: 976 - DNA, 147 bp, synthetic construct
  misc_feature 1..147: note = TC13P2_pool4_14
  source 1..147: mol_type = other DNA
gagatcatgtcctaaccctgatcctcttgtcccacagatatccagaaccctctaagaagttcctgcttatttacttcatagaatgtggttagagacatgaccctgccgtgtaccagctgagagactctaaatccagtgacaagtctg

SEQ ID NO: 977 - DNA, 147 bp, synthetic construct
  misc_feature 1..147: note = TC13P2_pool4_15
  source 1..147: mol_type = other DNA
gagatcatgtcctaaccctgatcctcttgtcccacagatatccagaaccctctaagaagttcctgcttattaacttccatgaatgtggttagagacatgaccctgccgtgtaccagctgagagactctaaatccagtgacaagtctg

SEQ ID NO: 978 - DNA, 147 bp, synthetic construct
  misc_feature 1..147: note = TC13P2_pool4_16
  source 1..147: mol_type = other DNA
gagatcatgtcctaaccctgatcctcttgtcccacagatatccagaaccctctaagaagttcctgcttttatacttccctgaatgtggttagagacatgaccctgccgtgtaccagctgagagactctaaatccagtgacaagtctg

SEQ ID NO: 979 - DNA, 147 bp, synthetic construct
  misc_feature 1..147: note = TC13P2_pool4_17
  source 1..147: mol_type = other DNA
gagatcatgtcctaaccctgatcctcttgtcccacagatatccagaaccctctaagaagttcctgcttattatcttccgagaatgtggttagagacatgaccctgccgtgtaccagctgagagactctaaatccagtgacaagtctg

SEQ ID NO: 980 - DNA, 147 bp, synthetic construct
  misc_feature 1..147: note = TC13P2_pool4_18
  source 1..147: mol_type = other DNA
gagatcatgtcctaaccctgatcctcttgtcccacagatatccagaaccctctaagaagttcctgctatatatcttccttgaatgtggttagagacatgaccctgccgtgtaccagctgagagactctaaatccagtgacaagtctg

SEQ ID NO: 981 - DNA, 147 bp, synthetic construct
  misc_feature 1..147: note = TC13P2_pool4_19
  source 1..147: mol_type = other DNA
gagatcatgtcctaaccctgatcctcttgtcccacagatatccagaaccctctaagaagttcctgctatatttcttcgaagaatgtggttagagacatgaccctgccgtgtaccagctgagagactctaaatccagtgacaagtctg

SEQ ID NO: 982 - DNA, 147 bp, synthetic construct
  misc_feature 1..147: note = TC13P2_pool4_20
  source 1..147: mol_type = other DNA
gagatcatgtcctaaccctgatcctcttgtcccacagatatccagaaccctctaagaagttcctgctattatacttcgcagaatgtggttagagacatgaccctgccgtgtaccagctgagagactctaaatccagtgacaagtctg

SEQ ID NO: 983 - DNA, 147 bp, synthetic construct
  misc_feature 1..147: note = TC13P2_pool4_21
  source 1..147: mol_type = other DNA
gagatcatgtcctaaccctgatcctcttgtcccacagatatccagaaccctctaagaagttcctgctaataatcttcggagaatgtggttagagacatgaccctgccgtgtaccagctgagagactctaaatccagtgacaagtctg

SEQ ID NO: 984 - DNA, 147 bp, synthetic construct
  misc_feature 1..147: note = TC13P2_pool4_22
  source 1..147: mol_type = other DNA
gagatcatgtcctaaccctgatcctcttgtcccacagatatccagaaccctctaagaagttcctgctataattcttcgttgaatgtggttagagacatgaccctgccgtgtaccagctgagagactctaaatccagtgacaagtctg

SEQ ID NO: 985 - DNA, 147 bp, synthetic construct
  misc_feature 1..147: note = TC13P2_pool4_23
  source 1..147: mol_type = other DNA
gagatcatgtcctaaccctgatcctcttgtcccacagatatccagaaccctctaagaagttcctgctaaatatcttctaagaatgtggttagagacatgaccctgccgtgtaccagctgagagactctaaatccagtgacaagtctg

SEQ ID NO: 986 - DNA, 147 bp, synthetic construct
  misc_feature 1..147: note = TC13P2_pool4_24
  source 1..147: mol_type = other DNA
gagatcatgtcctaaccctgatcctcttgtcccacagatatccagaaccctctaagaagttcctgctaaaatacttctcagaatgtggttagagacatgaccctgccgtgtaccagctgagagactctaaatccagtgacaagtctg

SEQ ID NO: 987 - DNA, 147 bp, synthetic construct
  misc_feature 1..147: note = TC13P2_pool4_25
  source 1..147: mol_type = other DNA
gagatcatgtcctaaccctgatcctcttgtcccacagatatccagaaccctctaagaagttcctgctattttacttctgtgaatgtggttagagacatgaccctgccgtgtaccagctgagagactctaaatccagtgacaagtctg

SEQ ID NO: 988 - DNA, 147 bp, synthetic construct
  misc_feature 1..147: note = TC13P2_pool4_26
  source 1..147: mol_type = other DNA
gagatcatgtcctaaccctgatcctcttgtcccacagatatccagaaccctctaagaagttcctgctttttatcttctttgaatgtggttagagacatgaccctgccgtgtaccagctgagagactctaaatccagtgacaagtctg

SEQ ID NO: 989 - DNA, 147 bp, synthetic construct
  misc_feature 1..147: note = TC13P2_pool4_27
  source 1..147: mol_type = other DNA
gagatcatgtcctaaccctgatcctcttgtcccacagatatccagaaccctctaagaagttcctgcttaaaatgatcaaagaatgtggttagagacatgaccctgccgtgtaccagctgagagactctaaatccagtgacaagtctg

SEQ ID NO: 990 - DNA, 147 bp, synthetic construct
  misc_feature 1..147: note = TC13P2_pool4_28
  source 1..147: mol_type = other DNA
gagatcatgtcctaaccctgatcctcttgtcccacagatatccagaaccctctaagaagttcctgctattaatgatcactgaatgtggttagagacatgaccctgccgtgtaccagctgagagactctaaatccagtgacaagtctg

SEQ ID NO: 991 - DNA, 147 bp, synthetic construct
  misc_feature 1..147: note = TC13P2_pool4_29
  source 1..147: mol_type = other DNA
gagatcatgtcctaaccctgatcctcttgtcccacagatatccagaaccctctaagaagttcctgcttaataagatcagagaatgtggttagagacatgaccctgccgtgtaccagctgagagactctaaatccagtgacaagtctg

SEQ ID NO: 992 - DNA, 147 bp, synthetic construct
  misc_feature 1..147: note = TC13P2_pool4_30
  source 1..147: mol_type = other DNA
gagatcatgtcctaaccctgatcctcttgtcccacagatatccagaaccctctaagaagttcctgcttaaatagatcattgaatgtggttagagacatgaccctgccgtgtaccagctgagagactctaaatccagtgacaagtctg

SEQ ID NO: 993 - DNA, 147 bp, synthetic construct
  misc_feature 1..147: note = TC13P2_pool4_31
  source 1..147: mol_type = other DNA
gagatcatgtcctaaccctgatcctcttgtcccacagatatccagaaccctctaagaagttcctgcttataaagatccaagaatgtggttagagacatgaccctgccgtgtaccagctgagagactctaaatccagtgacaagtctg

SEQ ID NO: 994 - DNA, 147 bp, synthetic construct
  misc_feature 1..147: note = TC13P2_pool4_32
  source 1..147: mol_type = other DNA
gagatcatgtcctaaccctgatcctcttgtcccacagatatccagaaccctctaagaagttcctgctttaaatgatccctgaatgtggttagagacatgaccctgccgtgtaccagctgagagactctaaatccagtgacaagtctg

SEQ ID NO: 995 - DNA, 147 bp, synthetic construct
  misc_feature 1..147: note = TC13P2_pool4_33
  source 1..147: mol_type = other DNA
gagatcatgtcctaaccctgatcctcttgtcccacagatatccagaaccctctaagaagttcctgctaatattgatccgtgaatgtggttagagacatgaccctgccgtgtaccagctgagagactctaaatccagtgacaagtctg

SEQ ID NO: 996 - DNA, 147 bp, synthetic construct
  misc_feature 1..147: note = TC13P2_pool4_34
  source 1..147: mol_type = other DNA
gagatcatgtcctaaccctgatcctcttgtcccacagatatccagaaccctctaagaagttcctgctttataagatccttgaatgtggttagagacatgaccctgccgtgtaccagctgagagactctaaatccagtgacaagtctg

SEQ ID NO: 997 - DNA, 147 bp, synthetic construct
  misc_feature 1..147: note = TC13P3_pool5_1
  source 1..147: mol_type = other DNA
gagatcatgtcctaaccctgatcctcttgtcccacagatatccagaaccctctaagaagttcctgctttaatagatcgaagaatgtggttagagacatgaccctgccgtgtaccagctgagagactctaaatccagtgacaagtctg

SEQ ID NO: 998 - DNA, 147 bp, synthetic construct
  misc_feature 1..147: note = TC13P3_pool5_2
  source 1..147: mol_type = other DNA
gagatcatgtcctaaccctgatcctcttgtcccacagatatccagaaccctctaagaagttcctgcttaatttgatcgctgaatgtggttagagacatgaccctgccgtgtaccagctgagagactctaaatccagtgacaagtctg

SEQ ID NO: 999 - DNA, 147 bp, synthetic construct
  misc_feature 1..147: note = TC13P3_pool5_3
  source 1..147: mol_type = other DNA
gagatcatgtcctaaccctgatcctcttgtcccacagatatccagaaccctctaagaagttcctgctaattttgatcggagaatgtggttagagacatgaccctgccgtgtaccagctgagagactctaaatccagtgacaagtctg

SEQ ID NO: 1000 - DNA, 147 bp, synthetic construct
  misc_feature 1..147: note = TC13P3_pool5_4
  source 1..147: mol_type = other DNA
gagatcatgtcctaaccctgatcctcttgtcccacagatatccagaaccctctaagaagttcctgctttatatgatcgtagaatgtggttagagacatgaccctgccgtgtaccagctgagagactctaaatccagtgacaagtctg

SEQ ID NO: 1001 - DNA, 147 bp, synthetic construct
  misc_feature 1..147: note = TC13P3_pool5_5
  source 1..147: mol_type = other DNA
gagatcatgtcctaaccctgatcctcttgtcccacagatatccagaaccctctaagaagttcctgctaattatgatctatgaatgtggttagagacatgaccctgccgtgtaccagctgagagactctaaatccagtgacaagtctg

SEQ ID NO: 1002 - DNA, 147 bp, synthetic construct
  misc_feature 1..147: note = TC13P3_pool5_6
  source 1..147: mol_type = other DNA
gagatcatgtcctaaccctgatcctcttgtcccacagatatccagaaccctctaagaagttcctgctttttaagatctcagaatgtggttagagacatgaccctgccgtgtaccagctgagagactctaaatccagtgacaagtctg

SEQ ID NO: 1003 - DNA, 147 bp, synthetic construct
  misc_feature 1..147: note = TC13P3_pool5_7
  source 1..147: mol_type = other DNA
gagatcatgtcctaaccctgatcctcttgtcccacagatatccagaaccctctaagaagttcctgctatttatgatctgagaatgtggttagagacatgaccctgccgtgtaccagctgagagactctaaatccagtgacaagtctg

SEQ ID NO: 1004 - DNA, 147 bp, synthetic construct
  misc_feature 1..147: note = TC13P3_pool5_8
  source 1..147: mol_type = other DNA
gagatcatgtcctaaccctgatcctcttgtcccacagatatccagaaccctctaagaagttcctgctaaattagatctttgaatgtggttagagacatgaccctgccgtgtaccagctgagagactctaaatccagtgacaagtctg

SEQ ID NO: 1005 - DNA, 147 bp, synthetic construct
  misc_feature 1..147: note = TC13P3_pool5_9
  source 1..147: mol_type = other DNA
gagatcatgtcctaaccctgatcctcttgtcccacagatatccagaaccctctaagaagttcctgctataaaagctcaaagaatgtggttagagacatgaccctgccgtgtaccagctgagagactctaaatccagtgacaagtctg

SEQ ID NO: 1006 - DNA, 147 bp, synthetic construct
  misc_feature 1..147: note = TC13P3_pool5_10
  source 1..147: mol_type = other DNA
gagatcatgtcctaaccctgatcctcttgtcccacagatatccagaaccctctaagaagttcctgcttataatgctcactgaatgtggttagagacatgaccctgccgtgtaccagctgagagactctaaatccagtgacaagtctg

SEQ ID NO: 1007 - DNA, 147 bp, synthetic construct
  misc_feature 1..147: note = TC13P3_pool5_11
  source 1..147: mol_type = other DNA
gagatcatgtcctaaccctgatcctcttgtcccacagatatccagaaccctctaagaagttcctgctaaaaatgctcagtgaatgtggttagagacatgaccctgccgtgtaccagctgagagactctaaatccagtgacaagtctg

SEQ ID NO: 1008 - DNA, 147 bp, synthetic construct
  misc_feature 1..147: note = TC13P3_pool5_12
  source 1..147: mol_type = other DNA
gagatcatgtcctaaccctgatcctcttgtcccacagatatccagaaccctctaagaagttcctgcttttattgctcatagaatgtggttagagacatgaccctgccgtgtaccagctgagagactctaaatccagtgacaagtctg

SEQ ID NO: 1009 - DNA, 147 bp, synthetic construct
  misc_feature 1..147: note = TC13P3_pool5_13
  source 1..147: mol_type = other DNA
gagatcatgtcctaaccctgatcctcttgtcccacagatatccagaaccctctaagaagttcctgctaataaagctccatgaatgtggttagagacatgaccctgccgtgtaccagctgagagactctaaatccagtgacaagtctg

SEQ ID NO: 1010 - DNA, 147 bp, synthetic construct
  misc_feature 1..147: note = TC13P3_pool5_14
  source 1..147: mol_type = other DNA
gagatcatgtcctaaccctgatcctcttgtcccacagatatccagaaccctctaagaagttcctgcttatttagctcccagaatgtggttagagacatgaccctgccgtgtaccagctgagagactctaaatccagtgacaagtctg

SEQ ID NO: 1011 - DNA, 147 bp, synthetic construct
  misc_feature 1..147: note = TC13P3_pool5_15
  source 1..147: mol_type = other DNA
gagatcatgtcctaaccctgatcctcttgtcccacagatatccagaaccctctaagaagttcctgcttattaagctccgtgaatgtggttagagacatgaccctgccgtgtaccagctgagagactctaaatccagtgacaagtctg

SEQ ID NO: 1012 - DNA, 147 bp, synthetic construct
  misc_feature 1..147: note = TC13P3_pool5_16
  source 1..147: mol_type = other DNA
gagatcatgtcctaaccctgatcctcttgtcccacagatatccagaaccctctaagaagttcctgcttttatagctccttgaatgtggttagagacatgaccctgccgtgtaccagctgagagactctaaatccagtgacaagtctg

SEQ ID NO: 1013 - DNA, 147 bp, synthetic construct
  misc_feature 1..147: note = TC13P3_pool5_17
  source 1..147: mol_type = other DNA
gagatcatgtcctaaccctgatcctcttgtcccacagatatccagaaccctctaagaagttcctgcttattatgctcgaagaatgtggttagagacatgaccctgccgtgtaccagctgagagactctaaatccagtgacaagtctg

SEQ ID NO: 1014 - DNA, 147 bp, synthetic construct
  misc_feature 1..147: note = TC13P3_pool5_18
  source 1..147: mol_type = other DNA
gagatcatgtcctaaccctgatcctcttgtcccacagatatccagaaccctctaagaagttcctgctatatatgctcgctgaatgtggttagagacatgaccctgccgtgtaccagctgagagactctaaatccagtgacaagtctg

SEQ ID NO: 1015 - DNA, 147 bp, synthetic construct
  misc_feature 1..147: note = TC13P3_pool5_19
  source 1..147: mol_type = other DNA
gagatcatgtcctaaccctgatcctcttgtcccacagatatccagaaccctctaagaagttcctgctatatttgctcggagaatgtggttagagacatgaccctgccgtgtaccagctgagagactctaaatccagtgacaagtctg

SEQ ID NO: 1016 - DNA, 147 bp, synthetic construct
  misc_feature 1..147: note = TC13P3_pool5_20
  source 1..147: mol_type = other DNA
gagatcatgtcctaaccctgatcctcttgtcccacagatatccagaaccctctaagaagttcctgctattatagctcgtagaatgtggttagagacatgaccctgccgtgtaccagctgagagactctaaatccagtgacaagtctg

SEQ ID NO: 1017 - DNA, 147 bp, synthetic construct
  misc_feature 1..147: note = TC13P3_pool5_21
  source 1..147: mol_type = other DNA
gagatcatgtcctaaccctgatcctcttgtcccacagatatccagaaccctctaagaagttcctgctaataatgctctaagaatgtggttagagacatgaccctgccgtgtaccagctgagagactctaaatccagtgacaagtctg

SEQ ID NO: 1018 - DNA, 147 bp, synthetic construct
  misc_feature 1..147: note = TC13P3_pool5_22
  source 1..147: mol_type = other DNA
gagatcatgtcctaaccctgatcctcttgtcccacagatatccagaaccctctaagaagttcctgctataattgctctctgaatgtggttagagacatgaccctgccgtgtaccagctgagagactctaaatccagtgacaagtctg

SEQ ID NO: 1019 - DNA, 147 bp, synthetic construct
  misc_feature 1..147: note = TC13P3_pool5_23
  source 1..147: mol_type = other DNA
gagatcatgtcctaaccctgatcctcttgtcccacagatatccagaaccctctaagaagttcctgctaaatatgctctgagaatgtggttagagacatgaccctgccgtgtaccagctgagagactctaaatccagtgacaagtctg

SEQ ID NO: 1020 - DNA, 147 bp, synthetic construct
  misc_feature 1..147: note = TC13P3_pool5_24
  source 1..147: mol_type = other DNA
gagatcatgtcctaaccctgatcctcttgtcccacagatatccagaaccctctaagaagttcctgctaaaatagctcttagaatgtggttagagacatgaccctgccgtgtaccagctgagagactctaaatccagtgacaagtctg

SEQ ID NO: 1021 - DNA, 147 bp, synthetic construct
  misc_feature 1..147: note = TC13P3_pool5_25
  source 1..147: mol_type = other DNA
gagatcatgtcctaaccctgatcctcttgtcccacagatatccagaaccctctaagaagttcctgctattttaggtcaatgaatgtggttagagacatgaccctgccgtgtaccagctgagagactctaaatccagtgacaagtctg

SEQ ID NO: 1022 - DNA, 147 bp, synthetic construct
  misc_feature 1..147: note = TC13P3_pool5_26
  source 1..147: mol_type = other DNA
gagatcatgtcctaaccctgatcctcttgtcccacagatatccagaaccctctaagaagttcctgctttttatggtcactgaatgtggttagagacatgaccctgccgtgtaccagctgagagactctaaatccagtgacaagtctg

SEQ ID NO: 1023 - DNA, 147 bp, synthetic construct
  misc_feature 1..147: note = TC13P3_pool5_27
  source 1..147: mol_type = other DNA
gagatcatgtcctaaccctgatcctcttgtcccacagatatccagaaccctctaagaagttcctgcttaaaatggtcagagaatgtggttagagacatgaccctgccgtgtaccagctgagagactctaaatccagtgacaagtctg

SEQ ID NO: 1024 - DNA, 147 bp, synthetic construct
  misc_feature 1..147: note = TC13P3_pool5_28
  source 1..147: mol_type = other DNA
gagatcatgtcctaaccctgatcctcttgtcccacagatatccagaaccctctaagaagttcctgctattaatggtcattgaatgtggttagagacatgaccctgccgtgtaccagctgagagactctaaatccagtgacaagtctg

SEQ ID NO: 1025 - DNA, 147 bp, synthetic construct
  misc_feature 1..147: note = TC13P3_pool5_29
  source 1..147: mol_type = other DNA
gagatcatgtcctaaccctgatcctcttgtcccacagatatccagaaccctctaagaagttcctgcttaataaggtccaagaatgtggttagagacatgaccctgccgtgtaccagctgagagactctaaatccagtgacaagtctg

SEQ ID NO: 1026 - DNA, 147 bp, synthetic construct
  misc_feature 1..147: note = TC13P3_pool5_30
  source 1..147: mol_type = other DNA
gagatcatgtcctaaccctgatcctcttgtcccacagatatccagaaccctctaagaagttcctgcttaaataggtccctgaatgtggttagagacatgaccctgccgtgtaccagctgagagactctaaatccagtgacaagtctg

SEQ ID NO: 1027 - DNA, 147 bp, synthetic construct
  misc_feature 1..147: note = TC13P3_pool5_31
  source 1..147: mol_type = other DNA
gagatcatgtcctaaccctgatcctcttgtcccacagatatccagaaccctctaagaagttcctgcttataaaggtccgagaatgtggttagagacatgaccctgccgtgtaccagctgagagactctaaatccagtgacaagtctg

SEQ ID NO: 1028 - DNA, 147 bp, synthetic construct
  misc_feature 1..147: note = TC13P3_pool5_32
  source 1..147: mol_type = other DNA
gagatcatgtcctaaccctgatcctcttgtcccacagatatccagaaccctctaagaagttcctgctttaaatggtccttgaatgtggttagagacatgaccctgccgtgtaccagctgagagactctaaatccagtgacaagtctg

SEQ ID NO: 1029 - DNA, 147 bp, synthetic construct
  misc_feature 1..147: note = TC13P3_pool5_33
  source 1..147: mol_type = other DNA
gagatcatgtcctaaccctgatcctcttgtcccacagatatccagaaccctctaagaagttcctgctaatattggtcgatgaatgtggttagagacatgaccctgccgtgtaccagctgagagactctaaatccagtgacaagtctg

SEQ ID NO: 1030 - DNA, 147 bp, synthetic construct
  misc_feature 1..147: note = TC13P3_pool5_34
  source 1..147: mol_type = other DNA
gagatcatgtcctaaccctgatcctcttgtcccacagatatccagaaccctctaagaagttcctgctttataaggtcgctgaatgtggttagagacatgaccctgccgtgtaccagctgagagactctaaatccagtgacaagtctg

SEQ ID NO: 1031 - DNA, 147 bp, synthetic construct
  misc_feature 1..147: note = TC13P3_pool6_1
  source 1..147: mol_type = other DNA
gagatcatgtcctaaccctgatcctcttgtcccacagatatccagaaccctctaagaagttcctgctttaataggtcggagaatgtggttagagacatgaccctgccgtgtaccagctgagagactctaaatccagtgacaagtctg

SEQ ID NO: 1032 - DNA, 147 bp, synthetic construct
  misc_feature 1..147: note = TC13P3_pool6_2
  source 1..147: mol_type = other DNA
gagatcatgtcctaaccctgatcctcttgtcccacagatatccagaaccctctaagaagttcctgcttaatttggtcgttgaatgtggttagagacatgaccctgccgtgtaccagctgagagactctaaatccagtgacaagtctg

SEQ ID NO: 1033 - DNA, 147 bp, synthetic construct
  misc_feature 1..147: note = TC13P3_pool6_3
  source 1..147: mol_type = other DNA
gagatcatgtcctaaccctgatcctcttgtcccacagatatccagaaccctctaagaagttcctgctaattttggtctaagaatgtggttagagacatgaccctgccgtgtaccagctgagagactctaaatccagtgacaagtctg

SEQ ID NO: 1034 - DNA, 147 bp, synthetic construct
  misc_feature 1..147: note = TC13P3_pool6_4
  source 1..147: mol_type = other DNA
gagatcatgtcctaaccctgatcctcttgtcccacagatatccagaaccctctaagaagttcctgctttatatggtctcagaatgtggttagagacatgaccctgccgtgtaccagctgagagactctaaatccagtgacaagtctg

SEQ ID NO: 1035 - DNA, 147 bp, synthetic construct
  misc_feature 1..147: note = TC13P3_pool6_5
  source 1..147: mol_type = other DNA
gagatcatgtcctaaccctgatcctcttgtcccacagatatccagaaccctctaagaagttcctgctaattatggtctgtgaatgtggttagagacatgaccctgccgtgtaccagctgagagactctaaatccagtgacaagtctg

SEQ ID NO: 1036 - DNA, 147 bp, synthetic construct
  misc_feature 1..147: note = TC13P3_pool6_6
  source 1..147: mol_type = other DNA
gagatcatgtcctaaccctgatcctcttgtcccacagatatccagaaccctctaagaagttcctgctttttaaggtcttagaatgtggttagagacatgaccctgccgtgtaccagctgagagactctaaatccagtgacaagtctg

SEQ ID NO: 1037 - DNA, 147 bp, synthetic construct
  misc_feature 1..147: note = TC13P3_pool6_7
  source 1..147: mol_type = other DNA
gagatcatgtcctaaccctgatcctcttgtcccacagatatccagaaccctctaagaagttcctgctatttatgttcaaagaatgtggttagagacatgaccctgccgtgtaccagctgagagactctaaatccagtgacaagtctg

SEQ ID NO: 1038 - DNA, 147 bp, synthetic construct
  misc_feature 1..147: note = TC13P3_pool6_8
  source 1..147: mol_type = other DNA
gagatcatgtcctaaccctgatcctcttgtcccacagatatccagaaccctctaagaagttcctgctaaattagttcactgaatgtggttagagacatgaccctgccgtgtaccagctgagagactctaaatccagtgacaagtctg

SEQ ID NO: 1039 - DNA, 147 bp, synthetic construct
  misc_feature 1..147: note = TC13P3_pool6_9
  source 1..147: mol_type = other DNA
gagatcatgtcctaaccctgatcctcttgtcccacagatatccagaaccctctaagaagttcctgctataaaagttcagagaatgtggttagagacatgaccctgccgtgtaccagctgagagactctaaatccagtgacaagtctg

SEQ ID NO: 1040 - DNA, 147 bp, synthetic construct
  misc_feature 1..147: note = TC13P3_pool6_10
  source 1..147: mol_type = other DNA
gagatcatgtcctaaccctgatcctcttgtcccacagatatccagaaccctctaagaagttcctgcttataatgttcattgaatgtggttagagacatgaccctgccgtgtaccagctgagagactctaaatccagtgacaagtctg

SEQ ID NO: 1041 - DNA, 147 bp, synthetic construct
  misc_feature 1..147: note = TC13P3_pool6_11
  source 1..147: mol_type = other DNA
gagatcatgtcctaaccctgatcctcttgtcccacagatatccagaaccctctaagaagttcctgctaaaaatgttccatgaatgtggttagagacatgaccctgccgtgtaccagctgagagactctaaatccagtgacaagtctg

SEQ ID NO: 1042 - DNA, 147 bp, synthetic construct
  misc_feature 1..147: note = TC13P3_pool6_12
  source 1..147: mol_type = other DNA
gagatcatgtcctaaccctgatcctcttgtcccacagatatccagaaccctctaagaagttcctgcttttattgttcccagaatgtggttagagacatgaccctgccgtgtaccagctgagagactctaaatccagtgacaagtctg

SEQ ID NO: 1043 - DNA, 147 bp, synthetic construct
  misc_feature 1..147: note = TC13P3_pool6_13
  source 1..147: mol_type = other DNA
gagatcatgtcctaaccctgatcctcttgtcccacagatatccagaaccctctaagaagttcctgctaataaagttccgtgaatgtggttagagacatgaccctgccgtgtaccagctgagagactctaaatccagtgacaagtctg

SEQ ID NO: 1044 - DNA, 147 bp, synthetic construct
  misc_feature 1..147: note = TC13P3_pool6_14
  source 1..147: mol_type = other DNA
gagatcatgtcctaaccctgatcctcttgtcccacagatatccagaaccctctaagaagttcctgcttatttagttcctagaatgtggttagagacatgaccctgccgtgtaccagctgagagactctaaatccagtgacaagtctg

SEQ ID NO: 1045 - DNA, 147 bp, synthetic construct
  misc_feature 1..147: note = TC13P3_pool6_15
  source 1..147: mol_type = other DNA
gagatcatgtcctaaccctgatcctcttgtcccacagatatccagaaccctctaagaagttcctgcttattaagttcgatgaatgtggttagagacatgaccctgccgtgtaccagctgagagactctaaatccagtgacaagtctg

SEQ ID NO: 1046 - DNA, 147 bp, synthetic construct
  misc_feature 1..147: note = TC13P3_pool6_16
  source 1..147: mol_type = other DNA
gagatcatgtcctaaccctgatcctcttgtcccacagatatccagaaccctctaagaagttcctgcttttatagttcgctgaatgtggttagagacatgaccctgccgtgtaccagctgagagactctaaatccagtgacaagtctg

SEQ ID NO: 1047 - DNA, 147 bp, synthetic construct
  misc_feature 1..147: note = TC13P3_pool6_17
  source 1..147: mol_type = other DNA
gagatcatgtcctaaccctgatcctcttgtcccacagatatccagaaccctctaagaagttcctgcttattatgttcggagaatgtggttagagacatgaccctgccgtgtaccagctgagagactctaaatccagtgacaagtctg

SEQ ID NO: 1048 - DNA, 147 bp, synthetic construct
  misc_feature 1..147: note = TC13P3_pool6_18
  source 1..147: mol_type = other DNA
gagatcatgtcctaaccctgatcctcttgtcccacagatatccagaaccctctaagaagttcctgctatatatgttcgttgaatgtggttagagacatgaccctgccgtgtaccagctgagagactctaaatccagtgacaagtctg

SEQ ID NO: 1049 - DNA, 147 bp, synthetic construct
  misc_feature 1..147: note = TC13P3_pool6_19
  source 1..147: mol_type = other DNA
gagatcatgtcctaaccctgatcctcttgtcccacagatatccagaaccctctaagaagttcctgctatatttgttctaagaatgtggttagagacatgaccctgccgtgtaccagctgagagactctaaatccagtgacaagtctg

SEQ ID NO: 1050 - DNA, 147 bp, synthetic construct
  misc_feature 1..147: note = TC13P3_pool6_20
  source 1..147: mol_type = other DNA
gagatcatgtcctaaccctgatcctcttgtcccacagatatccagaaccctctaagaagttcctgctattatagttctcagaatgtggttagagacatgaccctgccgtgtaccagctgagagactctaaatccagtgacaagtctg

SEQ ID NO: 1051 - DNA, 147 bp, synthetic construct
  misc_feature 1..147: note = TC13P3_pool6_21
  source 1..147: mol_type = other DNA
gagatcatgtcctaaccctgatcctcttgtcccacagatatccagaaccctctaagaagttcctgctaataatgttctgagaatgtggttagagacatgaccctgccgtgtaccagctgagagactctaaatccagtgacaagtctg

SEQ ID NO: 1052 - DNA, 147 bp, synthetic construct
  misc_feature 1..147: note = TC13P3_pool6_22
  source 1..147: mol_type = other DNA
gagatcatgtcctaaccctgatcctcttgtcccacagatatccagaaccctctaagaagttcctgctataattgttctttgaatgtggttagagacatgaccctgccgtgtaccagctgagagactctaaatccagtgacaagtctg

SEQ ID NO: 1053 - DNA, 147 bp, synthetic construct
  misc_feature 1..147: note = TC13P3_pool6_23
  source 1..147: mol_type = other DNA
gagatcatgtcctaaccctgatcctcttgtcccacagatatccagaaccctctaagaagttcctgctaaatattatcaaagaatgtggttagagacatgaccctgccgtgtaccagctgagagactctaaatccagtgacaagtctg

SEQ ID NO: 1054 - DNA, 147 bp, synthetic construct
  misc_feature 1..147: note = TC13P3_pool6_24
  source 1..147: mol_type = other DNA
gagatcatgtcctaaccctgatcctcttgtcccacagatatccagaaccctctaagaagttcctgctaaaatatatcacagaatgtggttagagacatgaccctgccgtgtaccagctgagagactctaaatccagtgacaagtctg

SEQ ID NO: 1055 - DNA, 147 bp, synthetic construct
  misc_feature 1..147: note = TC13P3_pool6_25
  source 1..147: mol_type = other DNA
gagatcatgtcctaaccctgatcctcttgtcccacagatatccagaaccctctaagaagttcctgctattttatatcagtgaatgtggttagagacatgaccctgccgtgtaccagctgagagactctaaatccagtgacaagtctg

SEQ ID NO: 1056 - DNA, 147 bp, synthetic construct
  misc_feature 1..147: note = TC13P3_pool6_26
  source 1..147: mol_type = other DNA
gagatcatgtcctaaccctgatcctcttgtcccacagatatccagaaccctctaagaagttcctgctttttattatcattgaatgtggttagagacatgaccctgccgtgtaccagctgagagactctaaatccagtgacaagtctg

SEQ ID NO: 1057 - DNA, 147 bp, synthetic construct
  misc_feature 1..147: note = TC13P3_pool6_27
  source 1..147: mol_type = other DNA
gagatcatgtcctaaccctgatcctcttgtcccacagatatccagaaccctctaagaagttcctgcttaaaattatccaagaatgtggttagagacatgaccctgccgtgtaccagctgagagactctaaatccagtgacaagtctg

SEQ ID NO: 1058 - DNA, 147 bp, synthetic construct
  misc_feature 1..147: note = TC13P3_pool6_28
  source 1..147: mol_type = other DNA
gagatcatgtcctaaccctgatcctcttgtcccacagatatccagaaccctctaagaagttcctgctattaattatccctgaatgtggttagagacatgaccctgccgtgtaccagctgagagactctaaatccagtgacaagtctg

SEQ ID NO: 1059 - DNA, 147 bp, synthetic construct
  misc_feature 1..147: note = TC13P3_pool6_29
  source 1..147: mol_type = other DNA
gagatcatgtcctaaccctgatcctcttgtcccacagatatccagaaccctctaagaagttcctgcttaataatatccgagaatgtggttagagacatgaccctgccgtgtaccagctgagagactctaaatccagtgacaagtctg

SEQ ID NO: 1060 - DNA, 147 bp, synthetic construct
  misc_feature 1..147: note = TC13P3_pool6_30
  source 1..147: mol_type = other DNA
gagatcatgtcctaaccctgatcctcttgtcccacagatatccagaaccctctaagaagttcctgcttaaatatatccttgaatgtggttagagacatgaccctgccgtgtaccagctgagagactctaaatccagtgacaagtctg

SEQ ID NO: 1061 - DNA, 147 bp, synthetic construct
  misc_feature 1..147: note = TC13P3_pool6_31
  source 1..147: mol_type = other DNA
gagatcatgtcctaaccctgatcctcttgtcccacagatatccagaaccctctaagaagttcctgcttataaatatcgaagaatgtggttagagacatgaccctgccgtgtaccagctgagagactctaaatccagtgacaagtctg

SEQ ID NO: 1062 - DNA, 147 bp, synthetic construct
  misc_feature 1..147: note = TC13P3_pool6_32
  source 1..147: mol_type = other DNA
gagatcatgtcctaaccctgatcctcttgtcccacagatatccagaaccctctaagaagttcctgctttaaattatcgctgaatgtggttagagacatgaccctgccgtgtaccagctgagagactctaaatccagtgacaagtctg

SEQ ID NO: 1063 - DNA, 147 bp, synthetic construct
  misc_feature 1..147: note = TC13P3_pool6_33
  source 1..147: mol_type = other DNA
gagatcatgtcctaaccctgatcctcttgtcccacagatatccagaaccctctaagaagttcctgctaatatttatcggtgaatgtggttagagacatgaccctgccgtgtaccagctgagagactctaaatccagtgacaagtctg

SEQ ID NO: 1064 - DNA, 147 bp, synthetic construct
  misc_feature 1..147: note = TC13P3_pool6_34
  source 1..147: mol_type = other DNA
gagatcatgtcctaaccctgatcctcttgtcccacagatatccagaaccctctaagaagttcctgctttataatatcgttgaatgtggttagagacatgaccctgccgtgtaccagctgagagactctaaatccagtgacaagtctg

SEQ ID NO: 1065 - DNA, 147 bp, synthetic construct
  misc_feature 1..147: note = TC13P4_pool7_1
  source 1..147: mol_type = other DNA
gagatcatgtcctaaccctgatcctcttgtcccacagatatccagaaccctctaagaagttcctgctttaatatatctaagaatgtggttagagacatgaccctgccgtgtaccagctgagagactctaaatccagtgacaagtctg

SEQ ID NO: 1066 - DNA, 147 bp, synthetic construct
  misc_feature 1..147: note = TC13P4_pool7_2
  source 1..147: mol_type = other DNA
gagatcatgtcctaaccctgatcctcttgtcccacagatatccagaaccctctaagaagttcctgcttaattttatctctgaatgtggttagagacatgaccctgccgtgtaccagctgagagactctaaatccagtgacaagtctg

SEQ ID NO: 1067 - DNA, 147 bp, synthetic construct
  misc_feature 1..147: note = TC13P4_pool7_3
  source 1..147: mol_type = other DNA
gagatcatgtcctaaccctgatcctcttgtcccacagatatccagaaccctctaagaagttcctgctaatttttatctgagaatgtggttagagacatgaccctgccgtgtaccagctgagagactctaaatccagtgacaagtctg

SEQ ID NO: 1068 - DNA, 147 bp, synthetic construct
  misc_feature 1..147: note = TC13P4_pool7_4
  source 1..147: mol_type = other DNA
gagatcatgtcctaaccctgatcctcttgtcccacagatatccagaaccctctaagaagttcctgctttatattatcttagaatgtggttagagacatgaccctgccgtgtaccagctgagagactctaaatccagtgacaagtctg

SEQ ID NO: 1069 - DNA, 147 bp, synthetic construct
  misc_feature 1..147: note = TC13P4_pool7_5
  source 1..147: mol_type = other DNA
gagatcatgtcctaaccctgatcctcttgtcccacagatatccagaaccctctaagaagttcctgctaattattctcaatgaatgtggttagagacatgaccctgccgtgtaccagctgagagactctaaatccagtgacaagtctg

SEQ ID NO: 1070 - DNA, 147 bp, synthetic construct
  misc_feature 1..147: note = TC13P4_pool7_6
  source 1..147: mol_type = other DNA
gagatcatgtcctaaccctgatcctcttgtcccacagatatccagaaccctctaagaagttcctgctttttaatctcacagaatgtggttagagacatgaccctgccgtgtaccagctgagagactctaaatccagtgacaagtctg

SEQ ID NO: 1071 - DNA, 147 bp, synthetic construct
  misc_feature 1..147: note = TC13P4_pool7_7
  source 1..147: mol_type = other DNA
gagatcatgtcctaaccctgatcctcttgtcccacagatatccagaaccctctaagaagttcctgctatttattctcagagaatgtggttagagacatgaccctgccgtgtaccagctgagagactctaaatccagtgacaagtctg

SEQ ID NO: 1072 - DNA, 147 bp, synthetic construct
  misc_feature 1..147: note = TC13P4_pool7_8
  source 1..147: mol_type = other DNA
gagatcatgtcctaaccctgatcctcttgtcccacagatatccagaaccctctaagaagttcctgctaaattatctcattgaatgtggttagagacatgaccctgccgtgtaccagctgagagactctaaatccagtgacaagtctg

SEQ ID NO: 1073 - DNA, 147 bp, synthetic construct
  misc_feature 1..147: note = TC13P4_pool7_9
  source 1..147: mol_type = other DNA
gagatcatgtcctaaccctgatcctcttgtcccacagatatccagaaccctctaagaagttcctgctataaaatctccaagaatgtggttagagacatgaccctgccgtgtaccagctgagagactctaaatccagtgacaagtctg

SEQ ID NO: 1074 - DNA, 147 bp, synthetic construct
  misc_feature 1..147: note = TC13P4_pool7_10
  source 1..147: mol_type = other DNA
gagatcatgtcctaaccctgatcctcttgtcccacagatatccagaaccctctaagaagttcctgcttataattctccctgaatgtggttagagacatgaccctgccgtgtaccagctgagagactctaaatccagtgacaagtctg

SEQ ID NO: 1075 - DNA, 147 bp, synthetic construct
  misc_feature 1..147: note = TC13P4_pool7_11
  source 1..147: mol_type = other DNA
gagatcatgtcctaaccctgatcctcttgtcccacagatatccagaaccctctaagaagttcctgctaaaaattctccgtgaatgtggttagagacatgaccctgccgtgtaccagctgagagactctaaatccagtgacaagtctg

SEQ ID NO: 1076 - DNA, 147 bp, synthetic construct
  misc_feature 1..147: note = TC13P4_pool7_12
  source 1..147: mol_type = other DNA
gagatcatgtcctaaccctgatcctcttgtcccacagatatccagaaccctctaagaagttcctgcttttatttctcctagaatgtggttagagacatgaccctgccgtgtaccagctgagagactctaaatccagtgacaagtctg

SEQ ID NO: 1077 - DNA, 147 bp, synthetic construct
  misc_feature 1..147: note = TC13P4_pool7_13
  source 1..147: mol_type = other DNA
gagatcatgtcctaaccctgatcctcttgtcccacagatatccagaaccctctaagaagttcctgctaataaatctcgatgaatgtggttagagacatgaccctgccgtgtaccagctgagagactctaaatccagtgacaagtctg

SEQ ID NO: 1078 - DNA, 147 bp, synthetic construct
  misc_feature 1..147: note = TC13P4_pool7_14
  source 1..147: mol_type = other DNA
gagatcatgtcctaaccctgatcctcttgtcccacagatatccagaaccctctaagaagttcctgcttatttatctcgcagaatgtggttagagacatgaccctgccgtgtaccagctgagagactctaaatccagtgacaagtctg

SEQ ID NO: 1079 - DNA, 147 bp, synthetic construct
  misc_feature 1..147: note = TC13P4_pool7_15
  source 1..147: mol_type = other DNA
gagatcatgtcctaaccctgatcctcttgtcccacagatatccagaaccctctaagaagttcctgcttattaatctcggtgaatgtggttagagacatgaccctgccgtgtaccagctgagagactctaaatccagtgacaagtctg

SEQ ID NO: 1080 - DNA, 147 bp, synthetic construct
  misc_feature 1..147: note = TC13P4_pool7_16
  source 1..147: mol_type = other DNA
gagatcatgtcctaaccctgatcctcttgtcccacagatatccagaaccctctaagaagttcctgcttttatatctcgttgaatgtggttagagacatgaccctgccgtgtaccagctgagagactctaaatccagtgacaagtctg

SEQ ID NO: 1081 - DNA, 147 bp, synthetic construct
  misc_feature 1..147: note = TC13P4_pool7_17
  source 1..147: mol_type = other DNA
gagatcatgtcctaaccctgatcctcttgtcccacagatatccagaaccctctaagaagttcctgcttattattctctaagaatgtggttagagacatgaccctgccgtgtaccagctgagagactctaaatccagtgacaagtctg

SEQ ID NO: 1082 - DNA, 147 bp, synthetic construct
  misc_feature 1..147: note = TC13P4_pool7_18
  source 1..147: mol_type = other DNA
gagatcatgtcctaaccctgatcctcttgtcccacagatatccagaaccctctaagaagttcctgctatatattctctctgaatgtggttagagacatgaccctgccgtgtaccagctgagagactctaaatccagtgacaagtctg

SEQ ID NO: 1083 - DNA, 147 bp, synthetic construct
  misc_feature 1..147: note = TC13P4_pool7_19
  source 1..147: mol_type = other DNA
gagatcatgtcctaaccctgatcctcttgtcccacagatatccagaaccctctaagaagttcctgctatattttctctgagaatgtggttagagacatgaccctgccgtgtaccagctgagagactctaaatccagtgacaagtctg

SEQ ID NO: 1084 - DNA, 147 bp, synthetic construct
  misc_feature 1..147: note = TC13P4_pool7_20
  source 1..147: mol_type = other DNA
gagatcatgtcctaaccctgatcctcttgtcccacagatatccagaaccctctaagaagttcctgctattatatctcttagaatgtggttagagacatgaccctgccgtgtaccagctgagagactctaaatccagtgacaagtctg

SEQ ID NO: 1085 - DNA, 147 bp, synthetic construct
  misc_feature 1..147: note = TC13P4_pool7_21
  source 1..147: mol_type = other DNA
gagatcatgtcctaaccctgatcctcttgtcccacagatatccagaaccctctaagaagttcctgctaataattgtcaaagaatgtggttagagacatgaccctgccgtgtaccagctgagagactctaaatccagtgacaagtctg

SEQ ID NO: 1086 - DNA, 147 bp, synthetic construct
  misc_feature 1..147: note = TC13P4_pool7_22
  source 1..147: mol_type = other DNA
gagatcatgtcctaaccctgatcctcttgtcccacagatatccagaaccctctaagaagttcctgctataatttgtcactgaatgtggttagagacatgaccctgccgtgtaccagctgagagactctaaatccagtgacaagtctg

SEQ ID NO: 1087 - DNA, 147 bp, synthetic construct
  misc_feature 1..147: note = TC13P4_pool7_23
  source 1..147: mol_type = other DNA
gagatcatgtcctaaccctgatcctcttgtcccacagatatccagaaccctctaagaagttcctgctaaatattgtcagagaatgtggttagagacatgaccctgccgtgtaccagctgagagactctaaatccagtgacaagtctg

SEQ ID NO: 1088 - DNA, 147 bp, synthetic construct
  misc_feature 1..147: note = TC13P4_pool7_24
  source 1..147: mol_type = other DNA
gagatcatgtcctaaccctgatcctcttgtcccacagatatccagaaccctctaagaagttcctgctaaaatatgtcatagaatgtggttagagacatgaccctgccgtgtaccagctgagagactctaaatccagtgacaagtctg

SEQ ID NO: 1089 - DNA, 147 bp, synthetic construct
  misc_feature 1..147: note = TC13P4_pool7_25
  source 1..147: mol_type = other DNA
gagatcatgtcctaaccctgatcctcttgtcccacagatatccagaaccctctaagaagttcctgctattttatgtccatgaatgtggttagagacatgaccctgccgtgtaccagctgagagactctaaatccagtgacaagtctg

SEQ ID NO: 1090 - DNA, 147 bp, synthetic construct
  misc_feature 1..147: note = TC13P4_pool7_26
  source 1..147: mol_type = other DNA
gagatcatgtcctaaccctgatcctcttgtcccacagatatccagaaccctctaagaagttcctgctttttattgtccctgaatgtggttagagacatgaccctgccgtgtaccagctgagagactctaaatccagtgacaagtctg

SEQ ID NO: 1091 - DNA, 147 bp, synthetic construct
  misc_feature 1..147: note = TC13P4_pool7_27
  source 1..147: mol_type = other DNA
gagatcatgtcctaaccctgatcctcttgtcccacagatatccagaaccctctaagaagttcctgcttaaaattgtccgagaatgtggttagagacatgaccctgccgtgtaccagctgagagactctaaatccagtgacaagtctg

SEQ ID NO: 1092 - DNA, 147 bp, synthetic construct
  misc_feature 1..147: note = TC13P4_pool7_28
  source 1..147: mol_type = other DNA
gagatcatgtcctaaccctgatcctcttgtcccacagatatccagaaccctctaagaagttcctgctattaattgtccttgaatgtggttagagacatgaccctgccgtgtaccagctgagagactctaaatccagtgacaagtctg

SEQ ID NO: 1093 - DNA, 147 bp, synthetic construct
  misc_feature 1..147: note = TC13P4_pool7_29
  source 1..147: mol_type = other DNA
gagatcatgtcctaaccctgatcctcttgtcccacagatatccagaaccctctaagaagttcctgcttaataatgtcgaagaatgtggttagagacatgaccctgccgtgtaccagctgagagactctaaatccagtgacaagtctg

SEQ ID NO: 1094 - DNA, 147 bp, synthetic construct
  misc_feature 1..147: note = TC13P4_pool7_30
  source 1..147: mol_type = other DNA
gagatcatgtcctaaccctgatcctcttgtcccacagatatccagaaccctctaagaagttcctgcttaaatatgtcgctgaatgtggttagagacatgaccctgccgtgtaccagctgagagactctaaatccagtgacaagtctg

SEQ ID NO: 1095 - DNA, 147 bp, synthetic construct
  misc_feature 1..147: note = TC13P4_pool7_31
  source 1..147: mol_type = other DNA
gagatcatgtcctaaccctgatcctcttgtcccacagatatccagaaccctctaagaagttcctgcttataaatgtcggagaatgtggttagagacatgaccctgccgtgtaccagctgagagactctaaatccagtgacaagtctg

SEQ ID NO: 1096 - DNA, 147 bp, synthetic construct
  misc_feature 1..147: note = TC13P4_pool7_32
  source 1..147: mol_type = other DNA
gagatcatgtcctaaccctgatcctcttgtcccacagatatccagaaccctctaagaagttcctgctttaaattgtcgttgaatgtggttagagacatgaccctgccgtgtaccagctgagagactctaaatccagtgacaagtctg

SEQ ID NO: 1097 - DNA, 147 bp, synthetic construct
  misc_feature 1..147: note = TC13P4_pool7_33
  source 1..147: mol_type = other DNA
gagatcatgtcctaaccctgatcctcttgtcccacagatatccagaaccctctaagaagttcctgctaatatttgtctatgaatgtggttagagacatgaccctgccgtgtaccagctgagagactctaaatccagtgacaagtctg

SEQ ID NO: 1098 - DNA, 147 bp, synthetic construct
  misc_feature 1..147: note = TC13P4_pool7_34
  source 1..147: mol_type = other DNA
gagatcatgtcctaaccctgatcctcttgtcccacagatatccagaaccctctaagaagttcctgctttataatgtctctgaatgtggttagagacatgaccctgccgtgtaccagctgagagactctaaatccagtgacaagtctg

SEQ ID NO: 1099 - DNA, 147 bp, synthetic construct
  misc_feature 1..147: note = TC13P4_pool8_1
  source 1..147: mol_type = other DNA
gagatcatgtcctaaccctgatcctcttgtcccacagatatccagaaccctctaagaagttcctgctttaatatgtctgagaatgtggttagagacatgaccctgccgtgtaccagctgagagactctaaatccagtgacaagtctg

SEQ ID NO: 1100 - DNA, 147 bp, synthetic construct
  misc_feature 1..147: note = TC13P4_pool8_2
  source 1..147: mol_type = other DNA
gagatcatgtcctaaccctgatcctcttgtcccacagatatccagaaccctctaagaagttcctgcttaattttgtctttgaatgtggttagagacatgaccctgccgtgtaccagctgagagactctaaatccagtgacaagtctg

SEQ ID NO: 1101 - DNA, 147 bp, synthetic construct
  misc_feature 1..147: note = TC13P4_pool8_3
  source 1..147: mol_type = other DNA
gagatcatgtcctaaccctgatcctcttgtcccacagatatccagaaccctctaagaagttcctgctaatttttttcaaagaatgtggttagagacatgaccctgccgtgtaccagctgagagactctaaatccagtgacaagtctg

SEQ ID NO: 1102 - DNA, 147 bp, synthetic construct
  misc_feature 1..147: note = TC13P4_pool8_4
  source 1..147: mol_type = other DNA
gagatcatgtcctaaccctgatcctcttgtcccacagatatccagaaccctctaagaagttcctgctttatattttcacagaatgtggttagagacatgaccctgccgtgtaccagctgagagactctaaatccagtgacaagtctg

SEQ ID NO: 1103 - DNA, 147 bp, synthetic construct
  misc_feature 1..147: note = TC13P4_pool8_5
  source 1..147: mol_type = other DNA
gagatcatgtcctaaccctgatcctcttgtcccacagatatccagaaccctctaagaagttcctgctaattattttcagtgaatgtggttagagacatgaccctgccgtgtaccagctgagagactctaaatccagtgacaagtctg

SEQ ID NO: 1104 - DNA, 147 bp, synthetic construct
  misc_feature 1..147: note = TC13P4_pool8_6
  source 1..147: mol_type = other DNA
gagatcatgtcctaaccctgatcctcttgtcccacagatatccagaaccctctaagaagttcctgctttttaatttcatagaatgtggttagagacatgaccctgccgtgtaccagctgagagactctaaatccagtgacaagtctg

SEQ ID NO: 1105 - DNA, 147 bp, synthetic construct
  misc_feature 1..147: note = TC13P4_pool8_7
  source 1..147: mol_type = other DNA
gagatcatgtcctaaccctgatcctcttgtcccacagatatccagaaccctctaagaagttcctgctatttattttccaagaatgtggttagagacatgaccctgccgtgtaccagctgagagactctaaatccagtgacaagtctg

SEQ ID NO: 1106 - DNA, 147 bp, synthetic construct
  misc_feature 1..147: note = TC13P4_pool8_8
  source 1..147: mol_type = other DNA
gagatcatgtcctaaccctgatcctcttgtcccacagatatccagaaccctctaagaagttcctgctaaattatttccctgaatgtggttagagacatgaccctgccgtgtaccagctgagagactctaaatccagtgacaagtctg

SEQ ID NO: 1107 - DNA, 147 bp, synthetic construct
  misc_feature 1..147: note = TC13P4_pool8_9
  source 1..147: mol_type = other DNA
gagatcatgtcctaaccctgatcctcttgtcccacagatatccagaaccctctaagaagttcctgctataaaatttccgagaatgtggttagagacatgaccctgccgtgtaccagctgagagactctaaatccagtgacaagtctg

SEQ ID NO: 1108 - DNA, 147 bp, synthetic construct
  misc_feature 1..147: note = TC13P4_pool8_10
  source 1..147: mol_type = other DNA
gagatcatgtcctaaccctgatcctcttgtcccacagatatccagaaccctctaagaagttcctgcttataattttccttgaatgtggttagagacatgaccctgccgtgtaccagctgagagactctaaatccagtgacaagtctg

SEQ ID NO: 1109 - DNA, 147 bp, synthetic construct
  misc_feature 1..147: note = TC13P4_pool8_11
  source 1..147: mol_type = other DNA
gagatcatgtcctaaccctgatcctcttgtcccacagatatccagaaccctctaagaagttcctgctaaaaattttcgatgaatgtggttagagacatgaccctgccgtgtaccagctgagagactctaaatccagtgacaagtctg

SEQ ID NO: 1110 - DNA, 147 bp, synthetic construct
  misc_feature 1..147: note = TC13P4_pool8_12
  source 1..147: mol_type = other DNA
gagatcatgtcctaaccctgatcctcttgtcccacagatatccagaaccctctaagaagttcctgcttttatttttcgcagaatgtggttagagacatgaccctgccgtgtaccagctgagagactctaaatccagtgacaagtctg

SEQ ID NO: 1111 - DNA, 147 bp, synthetic construct
  misc_feature 1..147: note = TC13P4_pool8_13
  source 1..147: mol_type = other DNA
gagatcatgtcctaaccctgatcctcttgtcccacagatatccagaaccctctaagaagttcctgctaataaatttcggtgaatgtggttagagacatgaccctgccgtgtaccagctgagagactctaaatccagtgacaagtctg

SEQ ID NO: 1112 - DNA, 147 bp, synthetic construct
  misc_feature 1..147: note = TC13P4_pool8_14
  source 1..147: mol_type = other DNA
gagatcatgtcctaaccctgatcctcttgtcccacagatatccagaaccctctaagaagttcctgcttatttatttcgtagaatgtggttagagacatgaccctgccgtgtaccagctgagagactctaaatccagtgacaagtctg

SEQ ID NO: 1113 - DNA, 147 bp, synthetic construct
  misc_feature 1..147: note = TC13P4_pool8_15
  source 1..147: mol_type = other DNA
gagatcatgtcctaaccctgatcctcttgtcccacagatatccagaaccctctaagaagttcctgcttattaatttctatgaatgtggttagagacatgaccctgccgtgtaccagctgagagactctaaatccagtgacaagtctg

SEQ ID NO: 1114 - DNA, 147 bp, synthetic construct
  misc_feature 1..147: note = TC13P4_pool8_16
  source 1..147: mol_type = other DNA
gagatcatgtcctaaccctgatcctcttgtcccacagatatccagaaccctctaagaagttcctgcttttatatttctctgaatgtggttagagacatgaccctgccgtgtaccagctgagagactctaaatccagtgacaagtctg

SEQ ID NO: 1115 - DNA, 147 bp, synthetic construct
  misc_feature 1..147: note = TC13P4_pool8_17
  source 1..147: mol_type = other DNA
gagatcatgtcctaaccctgatcctcttgtcccacagatatccagaaccctctaagaagttcctgcttattattttctgagaatgtggttagagacatgaccctgccgtgtaccagctgagagactctaaatccagtgacaagtctg

SEQ ID NO: 1116 - DNA, 147 bp, synthetic construct
  misc_feature 1..147: note = TC13P4_pool8_18
  source 1..147: mol_type = other DNA
gagatcatgtcctaaccctgatcctcttgtcccacagatatccagaaccctctaagaagttcctgctatatattttctttgaatgtggttagagacatgaccctgccgtgtaccagctgagagactctaaatccagtgacaagtctg

(19) United States
(12) Patent Application Publication
(10) Pub. No.: US 2026/0028617 A1
(43) Pub. Date: Jan. 29, 2026

(54) TALE BASE EDITORS FOR GENE AND CELL THERAPY

(71) Applicant: CELLECTIS SA, Paris (FR)

(72) Inventors: Alexandre JUILLERAT, New York, NY (US); Ming YANG, New York, NY (US); Alex BOYNE, New York, NY (US); Maria FEOLA, New York, NY (US); Julien VALTON, Paris (FR)

(21) Appl. No.: 18/867,238

(22) PCT Filed: Jun. 2, 2023
(86) PCT No.: PCT/EP2023/064836

§ 371 Date: Nov. 19, 2024

Related U.S. Application Data
(60) Provisional application No. 63/348,530, filed on Jun. 3, 2022.

(30) Foreign Application Priority Data
Oct. 28, 2022 (EP) 22204585.8
Nov. 29, 2022 (DK) PA2022 01085
May 16, 2023 (DK) PA2023 70240

(51) Int. Cl. C12N 15/10; C12N 9/78

(52) U.S. Cl. CPC C12N 15/102; C12N 9/78; C07K 2319/80; C12Y 305/04005

(57) ABSTRACT

The present invention relates to methods using base editors for efficiently genetically engineer cells, especially primary hematopoietic stem cells (HSCs) and primary immune cells. In particular, the invention is directed to rules for designing highly active and specific TALE-base editors displaying improved on-target/off-target activity ratios useful to manufacture complex gene edited cells of therapeutic grade or to perform in-vivo gene therapy. The resulting TALE-base editors can be used alone or in combination with rare-cutting endonucleases in various gene therapy approaches.

### FIELD OF THE INVENTION

The present invention relates to methods using base editors for efficiently genetically engineer cells, especially primary hematopoietic stem cells (HSCs) and primary immune cells. In particular, the invention is directed to rules for designing highly active and specific TALE-base editors displaying improved on-target/off-target activity ratios useful to manufacture complex gene edited cells of therapeutic grade or to perform in-vivo gene therapy. The resulting TALE-base editors can be used alone or in combination with rare-cutting endonucleases in various gene therapy approaches.

### BACKGROUND OF THE INVENTION

Artificial transcription-activator-like effectors (TALE) form a special class of proteins that can bind DNA originally derived from the phytopathogenic bacterial genus Xanthomonas [Kay S. et al. (2007) A bacterial effector acts as a plant transcription factor and induces a cell size regulator. Science 318: 648-651]. Artificial TALE proteins have emerged to be versatile and sequence specific gene tools offering flexible applications upon elucidation of a DNA recognition ‘code’, linking the amino-acid sequence of the TALE with its bound genomic DNA sequence [Moscou J. M. et al. (2009) A Simple Cipher Governs DNA Recognition by TAL Effectors. Science. 326:1501].

TALE binding is driven by a series of 33 to 35 amino-acid-long repeats that differ at essentially two positions, the so-called repeat variable dipeptide (RVD). Each base of one strand in the DNA target is contacted by a single repeat, with predictable specificity resulting from the linear arrangement of RVDs. The biochemical structure-function studies suggest that the amino acid present at position 13 uniquely identifies a nucleotide on the DNA target major groove [Deng D., et al. (2012) Structural basis for sequence-specific recognition of DNA by TAL effectors. Science 335:720-723; Stella S., et al. (2013) Structure of the AvrBs3-DNA complex provides new insights into the initial thymine-recognition mechanism. Acta Crystallogr Sect. D. Bio.l Crystallogr. 69(9):1707-1716]. This DNA-protein interaction unit is stabilized by the amino acid at position 12. For the creation of TALEs with variable precision and binding affinity, six conventional RVDs are generally used (NG, HD, NI, NK, NH, and NN). HD and NG are associated with cytosine (C) and thymine (T) respectively. NN is a degenerate RVD showing binding affinity for both guanine (G) and adenine (A), but its specificity for guanine is reported to be stronger. RVD NI binds with A and NK binds with G. It is worth noting that the binding affinity of TALE is influenced by the methylation status of the target DNA sequence [Streubel J, et al. (2012) TAL effector RVD specificities and efficiencies. Nat Biotechnol 30(7):593-595.]. Methylated cytosine is not efficiently bound by the canonical RVDs. However, they can be accommodated by a certain degree of degeneracy in TALEs as described by Valton J, et al. [Overcoming transcription activator-like effector (TALE) DNA binding domain sensitivity to cytosine methylation (2012) J. Biol. Chem. 287(46):38427-38432]. This code was adopted to effectively engineer TALE DNA-binding scaffold specificity via modular assembly in order to form different associations of TALE proteins with various enzymatic domains, such as transcriptional activators, repressors, base editors or nucleases with potential ability to act on genomic sequences [Voytas et al. (2011) TAL effectors: Customizable proteins for DNA targeting. Science. 333(6051):1843-6].

TALE-base editors (BE) have more recently emerged as fusions of TALE with deaminases, and sometimes, to other DNA repair proteins. Base editor catalytic domains can introduce single-nucleotide variants at desired loci in DNA (nuclear or organellar) or RNA of both dividing and non-dividing cells. Broadly, DNA base editors may be categorized into cytosine base editors (CBEs), adenine base editors (ABEs), C-to-G base editors (CGBEs), dual-base editors and organellar base editors.

Mok et al. [A bacterial cytidine deaminase toxin enables CRISPR-free mitochondrial base editing (2020) Nature. 583:631-637] recently developed a base editing approach by fusing TALE binding domains with the bacterial cytidine deaminase toxin, DddAtox, to demonstrate in vitro efficient C-to-T base conversions on mitochondrial genomes. In this approach, DddAtox was split into non-toxic halves that have respectively been fused to the C-terminus of paired (left and right) TALE binding domains, to form heterodimeric TALE base editors.

In such setting, the deaminase DddAtox becomes active when its two halves are brought together close enough by the TALE binding domains recognizing predetermined target DNA sequences in the genome by forming a functional heterodimer cytosine deaminase that converts C bases located between the two binding sites into T. Such DddA-TALE fusion deaminase constructs have so far achieved mitochondrial DNA editing in mice [Lee, H., et al. (2021) Mitochondrial DNA editing in mice with DddA-TALE fusion deaminases. Nat Commun 12:1190].

However, mitochondrial genomes are much smaller than nuclear genomes of human cells.

In human cells, especially immune therapeutic cells, the use of such base-editors has revealed to be very challenging. Especially in human gene therapy, the definition of the editing window to induce C-to-T base editing at the target site becomes of critical importance to avoid undesired substitution of any C bases located elsewhere into the proximal genomic region.

Depending on the sequences to be targeted in the genome and their intrinsic variability in human populations, TALE-base editors need further refinements for leveraging their activity and reducing the risk of potential off-target substitutions.

As shown in the experimental section herein, the inventors have performed extensive investigations to define rules that allowed to determine the best target genomic sequences in correlation with the design of efficient TALE base editors. They combined screening of dozens of TALE base editors targeting various endogenous loci with the development of a medium/high throughput cell-based assay that would leverage biases due confounding effects such as epigenomic factors or modifications. This approach relied on creating a pool of cells containing artificial targets for the base editor. The cells were generated by inserting a collection (30 to 191 members) of carefully designed BE target sequences into a predefined genomic locus. The pool of cells was then treated with various TALE base editors, generating gene edits on the collection of the different target sequences. Next generation sequencing (NGS) analysis of the editing frequencies on the BE targets allowed to better characterize the TALE base editors activity and substrate specificity within the editing window. The accumulated knowledge was then used to create new TALE base editors scaffolds referred to herein as “TALEB” that efficiently knocked-out several genes in primary T-cells, especially the CD52 gene (up to 87% phenotypically and 86% editing at the genomic level) and β2m gene, a potential target gene for allogeneic CAR T-cell adoptive therapies. The knowledge gained from this study shed lights on the editing guidelines and rules helped developing the TALE base editors of the present invention and their applications to therapeutic immune cells. Beyond the new scaffolds TALEB the invention offers a platform for rational design of TALE base editors of higher therapeutic grade based on the selection of appropriate endogenous genomic targets.

### SUMMARY OF THE INVENTION

TALE recombinant DddA-derived cytosine base editors are heterodimers generated by fusion of transcription activator-like effector array proteins (TALE), split-DddA deaminase halves, together with an uracil glycosylase inhibitor (UGI). It is a recent improvement of the available base editor tools, which can directly edit double strand DNA, converting cytosine (C) to thymine (T). Such TALE base editors have been used to create edits in mitochondria and generate inheritable modifications. However, the editing rules for this particular base editors have not been fully elucidated. To further dissect the editing rules of TALE base editors, the present inventors have exploited nuclease based targeted knock-in technology and created a pool of cells, each harboring unique BE target sequences at the same genomic locus. These cells were then treated with TALE base editors, followed by NGS analysis for the mutations pattern on the target sequences. As shown in the experimental section herein, such methods allowed to generate a large and diverse pool of TALE base editors targets and to gain in depth insight of the editing rules in cellulo, while excluding the confounding factors such as epigenetic and microenvironmental differences among different genomic loci. With the knowledge gained from this innovative approach, the inventors have designed new scaffolds referred to as “TALEB” against a range of endogenous genes, such as those encoding CD52, TCR, B2M and PD1 which are useful to knockout in therapeutic immune cells.

As an aspect, the present invention is drawn to the identification of target sequences in the genome that specifically allow a specific focus of TALE base editors on a desired cytosine (C) to be converted into thymine (T), while limiting off target mutations. Such “sharper” target sequences are defined by:

5′-T₀-N_{left}-N_{y}-RTC-Nₓ-Nr_{ight}-A₀-3′;
and
5′-T₀-N_{left}-Nₓ-GAY-N_{y}-N_{right}-A₀-3′

wherein
N can be A, T, C or G
R can be G or A, preferably G
Y can be C or T, preferably C;
N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
x=2 to 6
y=6 to 10
with preferably x+y≥11, more preferably x+y=12.

As shown in FIG. 3, the above general formula deciphers a surface on the double strand DNA accessible to the deaminase which has an approximate length of 7 nucleotides (L=0.34 nm×6=2.4 nm), which represents the best target window in a genomic sequence to target the desired C with a TALE base editor. This surface has a circular arc f=4×34.3°=136.8°=2.38 radian (angle over 5 nucleotide bases). Assuming that the radius of the double DNA helix is about 1 nm, then that surface targeting C corresponds to L×R×f=2.4×1×2.38=5.71 nm².

Thus, that target surface, framed by the diagonals linking the bases at positions N11, N-13 (opposite strand) et N9, N-9 (opposite strand) is of about 4.87 nm² when N_{left} and N_{right} are spaced by 15 bases.

As per the experiments shown in the examples, more specific TALE base editors, such as the illustrated “TALEB” of the present invention can be designed to more specifically target genomic sequences defined as

5′-T₀-N_{left}-N_{y}-RTCC-Nₓ-N_{right}-A₀-3′;
and
5′-T₀-N_{left}-Nₓ-GGAY-N_{y}-N_{right}-A₀- 3′

wherein
N can be A, T, C or G
R can be G or A, preferably G
Y can be C or T, preferably C; N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
x=1 to 5
y=5 to 9

The spacer, defined as the number of base pairs between the binding sites N_{left} and N_{right} are preferably 13 or 15 bp.

As from the experiments, the TALE base editor monomers of the present invention comprising TALE C-terminus comprising less than 40 amino acids, such as the C40 and C11 illustrated herein, show higher specificity on target sequences comprising a spacer of 15 bp. TALEB monomers comprising TALE C-terminus comprising less than 12 amino acids, such as the C11 illustrated herein, showed highest specificity, especially in conjunction with a spacer of 15 pb, but also with a spacer of 13 bp. Thus such TALE base editor monomers are particularly suited to target sequences comprising a spacer of about 10 to 20 pb, more preferably from 13 to 16 pb, and even more preferably from 12 to 15 bp.

The TALE base editor monomers comprising a TALE C-terminus of less than 12 amino acids, in particular the C11 illustrated herein, also appeared to be more discriminating when a stretch of C, such as at least two CC, three CCC or four CCCC was present in the target sequence, this stretch of C being or not but preferably being preceded by T, and the first C being generally that to be converted into T (C>T) by the TALE base editor.

One benefit of such embodiment is the possibility offered by the TALE base editors of the present invention to target genomic sequence that would not present a “T” immediately before the C to be edited, or that presents a stretch of CCC following such “T”. The present invention thus broadens the number of sequences that can be edited with TALE base editors.

Given these findings, the invention provides a method for designing TALE base editors that sharply target C positions in genetic sequences, said method comprising one of the following steps:
i) Identifying a target sequence as defined above into a genome;
ii) Synthetizing polynucleotide sequences encoding left and right TALE binding polypeptides that bind the N_{left} and N_{right} polynucleotide sequences, respectively.
iii) fusing said polynucleotide sequence encoding left TALE binding polypeptide to a polynucleotide encoding a N-terminal split DddAtox;
iv) fusing said polynucleotide sequence encoding right TALE binding polypeptide to a polynucleotide encoding a C terminal split DddAtox;
v) fusing a polynucleotide sequence encoding a polypeptide preventing uracyl glycosylation, such as UGI (Uracil glycosylase inhibitor) to at least one polynucleotide sequence encoding said polynucleotide sequence resulting from ii) and iii).
vi) Optionally, co-expressing the two resulting polynucleotide sequences to obtain a TALE base editor heterodimer.

According to some aspects, said left and right TALE binding polypeptides comprise a C-terminus of 1 to 50 amino acids, preferably 8 to 40, more preferably 10 to 30, even more preferably about 11 amino acids or about 40 amino acids.

According to some aspects, said left and right TALE binding polypeptides comprise a C-terminus of about 13 or 40 amino acids from the original AvrBs3 TALE protein, which is generally at least 90%, 95% or 99% identical to SEQ ID NO:4.

According to some aspects, the Nter and/or Cter member(s) of said split DddAtox comprise(s) at least one mutation that decreases the affinity of the two splits DddAtox members for each other, in order to avoid TALE independent a specific binding of the DddAtox in the genome, thereby increasing TALE base editor specificity.

Accordingly to some aspects, the invention can be regarded as a method for introducing a mutation into the genome of a cell, comprising the step of introducing or expressing into the cell a TALE base editor consisting of a heterodimeric fusion of a left and right TALE binding polypeptides having a C-terminal domain of about 1 to 50 amino acids, with respectively a C terminal and N terminal split DddATox, wherein said heterodimeric TALE base editor binds a genomic sequence as previously defined.

As preferred embodiments are methods of gene editing using the TALE base editors of the present invention in gene therapy, especially to engineer and manufacture primary cells ex-vivo, more particularly HSCs and immune cells, such as T-cells and NK-cells for cell therapy. The manufacturing of the therapeutic cells more particularly comprises steps where base editors are used to make them allogeneic and/or stealthy to the patient's immune system, such as by disrupting TCR or B2M genes, and other steps where rare-cutting endonucleases are used for the purpose of gene targeting insertions or replacement, such as for instance at immune checkpoint genes loci.

Such manufacturing strategies are particularly effective when they combine TCR inactivation by using a base editor and insertion/replacement of a chimeric antigen receptor or recombinant TCR at a different locus such as B2M or PD1. Another example is the opposite strategy, B2M inactivation by using a base editor and insertion at the TCR locus.

One preferred method comprises the step of making the cells resistant to an immunosuppressive drug by inactivating a gene, such as CD52, by using a base editor and integrating an exogenous polynucleotide sequence at another locus by using a rare-cutting endonuclease. Such steps can be performed at the same time, by co-electroporating immune cells or precursors thereof with a base editing reagent and at least one nuclease reagent.

In this respect the present invention provides specific reagents and target sequences to successfully achieve the manufacturing of such therapeutic immune cells as well as various examples of TALE-base editor proteins designed according to the principles and rules of the present invention.

The TALE base editors as per the present invention can also be used for in-vivo gene therapy to correct mutations or inactivate inherited deficient genes, such as ApoC3 in liver cells.

The invention encompasses vectors comprising the polynucleotide sequences as well as the polypeptide sequences or reagents obtainable by the present invention, as well as their use for cell transformation and gene modification.

### DESCRIPTION OF FIGURES AND TABLES

FIG. 1: Schematic representation of the TALE base editors of the present invention. TALEBs are composed of the N-terminal part of a TALE such as a N152 truncation of AvrBs3, repeats arrays, a C-terminal part of a TALE preferably an AvrBs3 C11 or C40 truncation, a split DddATox (ex: at position G1397) and a UGI (Uracil glycosylase inhibitor).

FIG. 2: A. Diagram showing distribution of the 37 TALE-nucleases tested in Example 2 based on their nuclease activity. B. Comparison of the activity of TALE-nuclease (Y axis) vs. TALE base editors (X-axis) frequency with respect to 37 TALE target sequences: there is no significant correlation between TALE-nuclease activity and TALE-base editing at those target sequences.

FIG. 3: A. 3D schematic representation of double stranded DNA structure showing the sites (black circles) that can be edited by the TALE base editor as per the interpretation of the experimental analysis provided herein with TALE base editors split DddATox heterodimers. B. 3D schematic representation of the surface that can be edited by the TALE base editors.

FIG. 4: A. Graphic representation of the frequency of indels (Y axis) vs % C-T conversion (Y avis) induced by the TALE base editors of the present invention. B. Percentage of editing purity. percentage of C-T conversion only on all conversions (“editing only”) or on all conversions and indels (“editing and indels”) detected within the spacer for each TALE base editors tested. C. Schematic representation of the different events induced by each Individual 37 TALE base editors of example 2. These figures are indicative of a very high final purity of the edited cell populations for all levels of activity induced by the TALE base editors.

FIG. 5: A. Design of first TALE base editors screening described in example 3. The pools of oligos comprise left and right homology arms of the TRAC locus, left and right binding sequence of T-25, and TC/GA sequence that is placed at different place within a 15 bp the spacer. After double transfection (TRAC TALE-Nuclease with ssODN pool, and T-25 base editor) genomic DNA is analysed by NGS. B. Representation of the different events (C-T conversion: Edition, Indels, other mutations, none) obtained on 2 different donors. C. Correlation between the donors of the C-T conversion frequency obtained on the bottom (left graph) or the top strand (right graph). D. Percentage of C-T conversion depending on the localization of C on either the upper strand (top graph) or the lower strand (lower graph).

FIG. 6: A. Design of second TALE base editors screening performed in example 3. The pool of oligos comprises left and right homology arms of the TRAC locus, left and right binding sequence of T-25, and TC/GA sequence that is placed at different place in spacers varying in length. In addition, a bare code (unique specific sequence) between right binding of TALE and Right homology arm is inserted for each spacer length. B. Representation of the different events (C-T conversions: “Edition”, Indels, other mutations, none) obtained on 2 different donors. C. Correlation between the donors of the C-T conversion frequency obtained on the bottom (left graph) or the top strand (right graph). D. Frequency of C-T conversion on the top strand (left graph) or bottom strand (right graph) depending on C-T position in the spacer and the length of the spacer.

FIG. 7: Heatmap of C-to-T conversion in function of the TC context (NTCCNN). N=2, independent T-cells donors demonstrating that a G or an A before the TC favored efficient BE-editing as per the experiments shown in Example 3.

FIG. 8: A. Schematic representation of the base editing strategy according to the invention to inactivate the CD52 gene to create therapeutic immune cells by mutating the splice acceptor site of CD52. B. Percentage of CD52 negative cells obtained with the indicated TALE base editors in Example 4. C. Frequency of C-T conversion (E) or Indels (I) obtained with the indicated TALE base editors.

FIG. 9: A. Schematic representation of the base editing strategy according to the invention to inactivate the CD52 gene to create therapeutic immune cells by mutating the signal peptide of CD52. B. Percentage of CD52 negative obtained with indicated TALE base editors. C. Frequency of C-T conversion obtained at the indicated position or indels. D. Frequency of the different sequence obtained post TALE base editors treatment (amino acid substitution are indicated in grey).

FIG. 10: Flow cytometry analysis (TCR X axis and CD52 Y axis) of primary T cells, untreated (upper panel), treated with TALEN targeting TRAC and CD52, (lower left panel), treated with TALEN targeting TRAC and TALEB targeting CD52 as per the present invention resulting from the experiments of Example 4.

FIG. 11: Diagram comparing translocation reads in primary T cells treated with either TALEN targeting TRAC and CD52, (TALEN+TALEN) or TALEN targeting TRAC and TALE-base editor targeting CD52 as per the present invention in Example 4.

FIGS. 12, 13 and 14: Schematic representation of a gene therapy method as per the present invention which may consist in using a sequence specific nuclease to insert a functional copy of a gene or a corrected sequence thereof in combination with a sequence specific base editor reagent that is used to inactivate residual endogenous sequences acting as a “proof reader”. In the illustrated situation, the correct sequence has been rewritten with respect to the wild type allele sequence by using alternative codons and introduced in the genome by using site-directed nuclease integration. Different outcomes (scenarios A to C) can be expected from this integration in the cell's genome, which is mainly operated by homologous recombination, depending on the degree of allelic replacement. A: Both the dominant mutated allele and the wild-type functional allele have been replaced resulting into a functional homozygote cell. B: only the dominant mutated allele has been replaced resulting into a functional heterozygote cell. C: none insertion has occurred and the heterozygote cell remains deficient. D: only the wild type allele has been replaced resulting into a still deficient heterozygote cell. In FIG. 14, the sequence specific base editor, such as a TALE base editors described in this specification, is introduced in the cell to inactivate the endogenous sequences (i.e. non rewritten sequences), which have not been replaced/corrected by the integration of the functional rewritten sequences.

FIG. 15: Schematic representation of the insertion of an artificial exon (Artex) site directed by a sequence specific endonuclease into an endogenous gene, so that exon expression is placed under the endogenous gene promoter. Such a strategy for corrected exon insertion can be combined with the introduction of a base editor to “proof-read” and inactivate non-corrected exons, as a particular embodiment of the method illustrated through the previous FIGS. 12 to 14.

FIGS. 16 and 17: As an embodiment of the gene therapy method of FIGS. 12 to 14, these figures illustrate combining a sequence specific endonuclease and base-editor, wherein a specific endonuclease can be co-electroporated with a DNA matrix encoding a therapeutic cassette comprising an exogenous promoter for its integration at a predetermined locus between exon 1 and exon 2 of a particular gene. Scenarios 1 to 4 correspond to the possible outcomes of the cassette integration with respect to the deficient endogenous exon 3 allele and the benefit of using a base editor to inactivate the expression of exon 3 to deal with each of these situations, either sequentially (as illustrated) or simultaneously (ex: co-transfection). In this example, for the sake of simplicity, it is assumed that the base editor edits both alleles. However, it possible that such editing can also discriminate the allele bearing the deleterious mutation.

FIGS. 18 and 19: As an embodiment of the gene therapy method of FIGS. 12 to 14, these figures illustrate the integration of a promoterless corrected copy of an exon which is placed under control of the endogenous promoter of the gene by Artex (as shown in FIG. 15), and the subsequent inactivation of the original deficient exon by base editing.

FIG. 20: A. Example of nuclease/base editor mediated gene therapy as per the present invention to correct dominant negative mutation occurring in exon 24 of PIK3CD causing ADPS1 through the endonuclease mediated integration of a promoterless therapeutic cDNA matrix encoding the corrected sequence of exons 2 to 24 via the Artex approach (FIG. 15). The expression of the original deficient exon 24, when not being prevented by the insertion itself, is inactivated by base editing as detailed in Example 5. With such method, all the reagents, in particular the site-specific endonuclease and the sequence specific base editor base can be introduced in the cell simultaneously, such as by co-electroporation. B. Schematic representation detailing the different elements constituting the therapeutic repair matrix.

FIG. 21: Schematic representation of the site-specific integration by Artex of a promoterless corrected copy of PIK3CD (including exon 24) into Intron 2 of that gene into an isolated HSC, and the subsequent inactivation of the original deficient exon by base editing as detailed in Example 5 by using a TALE base editor as per the present invention.

FIG. 22: schematic representation of the artificial STAT3 TALEB target sequences including 5, 7, 11, 13, 15 and 17 bp spacer length/editing window to be inserted at the TRAC locus to test C-to-T editing efficiency as detailed in example 6.

FIG. 23: detailed representation of the TALEB assayed in example 6 for optimal C-to-T editing efficiency including the alternative TALE C-terminal “linkers” CO, C11 and C40.

FIG. 24: Diagram analysis of the sequencing data obtained from the NGS analysis resulting from the experiment of Example 6 evaluating the CO, C11 and C40 TALEB scaffolds with respect to the different spacer lengths. A: edited targets with 5 bp spacers. B: edited targets with 7 bp spacers. C: edited targets with 9 bp spacers. D: edited targets with 11 bp spacers. E: edited targets with 13 bp spacers: F: edited targets with 15 bp spacers: G: edited targets with 17 bp spacers.

FIG. 25: Diagram analysis of the sequencing data obtained from the NGS analysis resulting from the experiment of Example 6 evaluating the combination of C11 and C40 heterodimers on targets with 15 pb spacer.

FIG. 26: schematic representation of the library of target sequences inserted at the TCR locus through the experiments of example 6 to test context variation around edited “TO” when using STAT3 TALEB scaffolds involving CO, C11 and C40 linker structures.

FIG. 27: positions that vary in the library of target sequences which is illustrated in FIG. 26.

FIG. 28: data analysis from bioinformatics determining the contribution of each surrounding base to the efficiency of C editing in the context of 15 bp spacer. A: using C40 TALEB scaffold. B: using C11 TALEB scaffold.

FIG. 29: data analysis from bioinformatics showing TCC->TTT efficacy depending on each base surrounding the TCC in the context of 15 bp spacer. A: using C40 TALEB scaffold. B: using C11 TALEB scaffold.

FIG. 30: data analysis from bioinformatics determining the contribution of each surrounding base to the efficiency of C editing in the context of 13 bp spacer. A: using C40 TALEB scaffold. B: using C11 TALEB scaffold.

FIG. 31: data analysis from bioinformatics showing TCC->TTT efficacy depending on each base surrounding the TCC in the context of 13 bp spacer. A: using C40 TALEB scaffold. B: using C11 TALEB scaffold.

FIG. 32: Results of the experiments detailed in example 7 regarding strategy of gene editing in T-cells combining TALEB (TCR KO) and TALEN (KI of HLAE using AAV matrix and homologous recombination). The results show efficient gene editing and avoidance of “AAV trapping” at the TRAC locus. A: diagram representation showing percentage of gene edited cells. B: Flow cytometry analysis comparing use of TALEN and TALEB to inactivate TCR in the presence of HLAE AAV matrix.

Table 1: 37 genomic target sequences used in Example 2.

Table 2: Sequences of the 2×15 individual ssODN used to identify editing windows with a 15 bp spacer in Example 3.

Table 3: Sequences of the 191 individual ssODN used to assess effect of spacer length on editing in Example 3.

Table 4: Sequences of individual ssODN used to assess the TC context in TALE base editors target sequences in Example 4.

Table 5: KO CD52 TALEB polypeptides and example of target polynucleotides as per the present invention.

Table 6: Predicted potential off-targeted site for the 4 TALEB targeting CD52 assessed in Example 4.

Table 7: List of TALEB target sequence windows following the rules of the present invention to introduce mutations in the TRAC gene.

Table 8: List of TALEB target sequence windows following the rules of the present invention to introduce mutations in the CD52 gene.

Table 9: List of TALEB target sequence windows following the rules of the present invention to introduce mutations in the PD1 gene.

Table 10: List of TALEB target sequence windows following the rules of the present invention to introduce mutations in the B2m gene.

Table 11: List of TALEB target sequence windows following the rules of the present invention to introduce mutations in the ApoC3 gene.

Table 12: Base editors target sites in Exon 1, 2 or 3 of PK13 gene as per the combined gene therapy (nuclease+base editor) method of the present invention illustrated in example 5 herein.

Table 13: Polypeptide sequences of the different TALE C-terminal length used in TALEB referred to as C40, C11 and CO backbones.

Table 14: TALEB heterodimers tested in Example 6 Table 15: Library of ssODN comprising 5′TC at 11 positions flanked by optimal spacer length (either a 13 or 15 bp spacer length) integrated at the TCR locus to be targeted by the STAT3 TALEB target.

Table 16: Library of ssODN to assess influence of the context around TC in the 15 bp spacer length in example 6.

Table 17: Library of ssODN to assess influence of the context around TC in the 13 bp spacer length in example 6.

Table 18: Polynucleotide and polypeptide sequences used in Example 7.

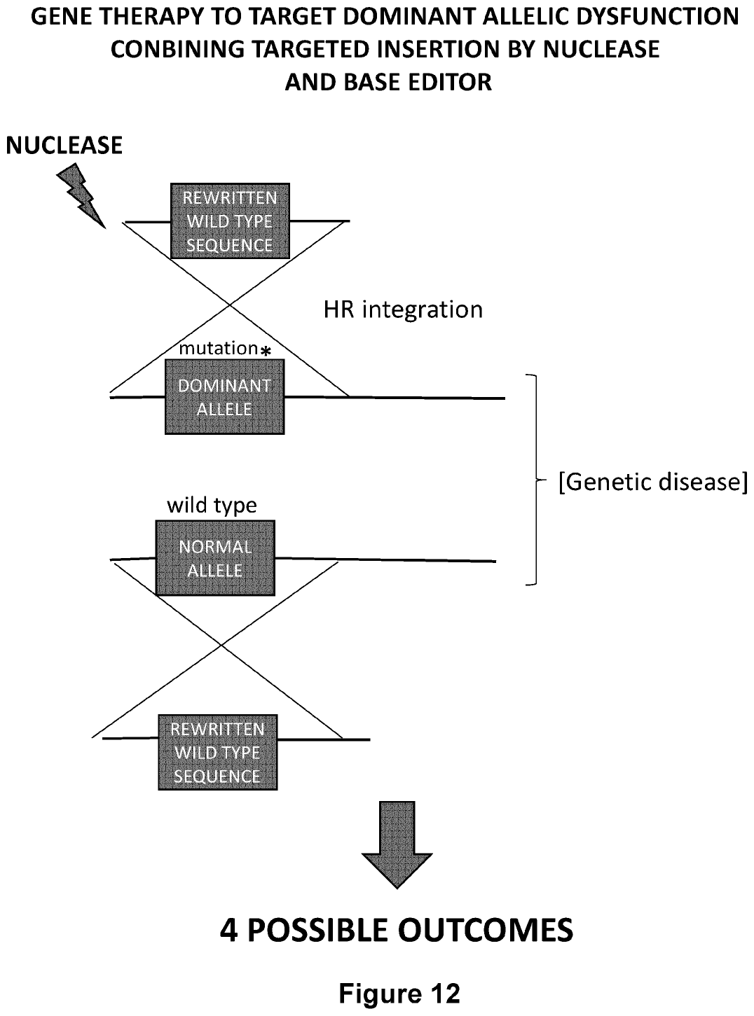

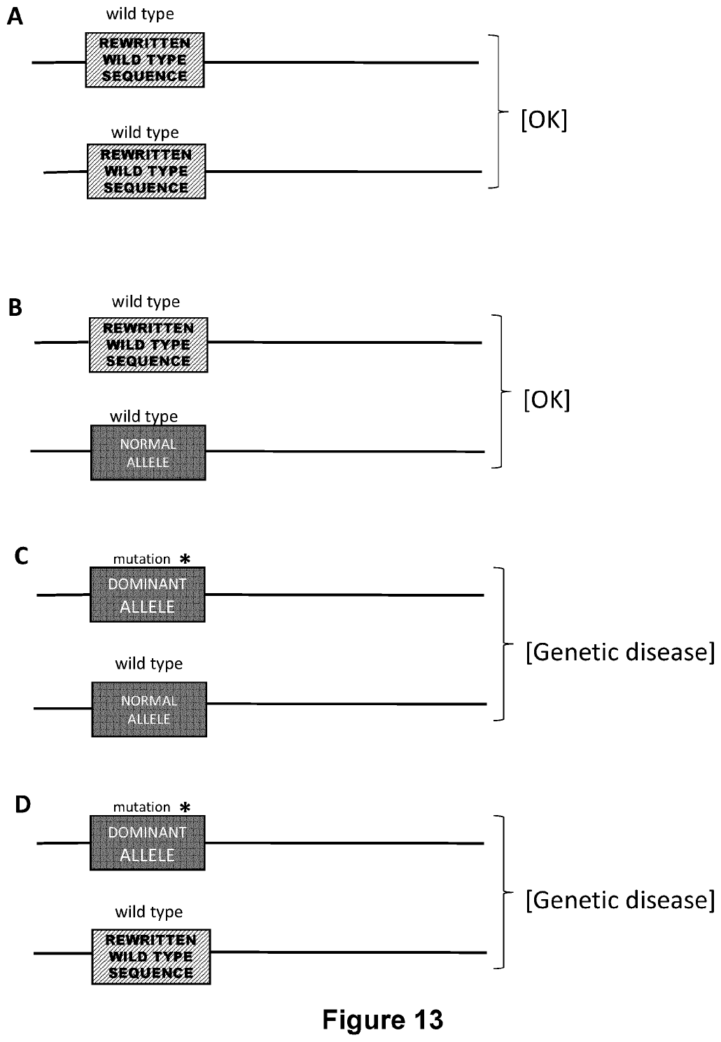

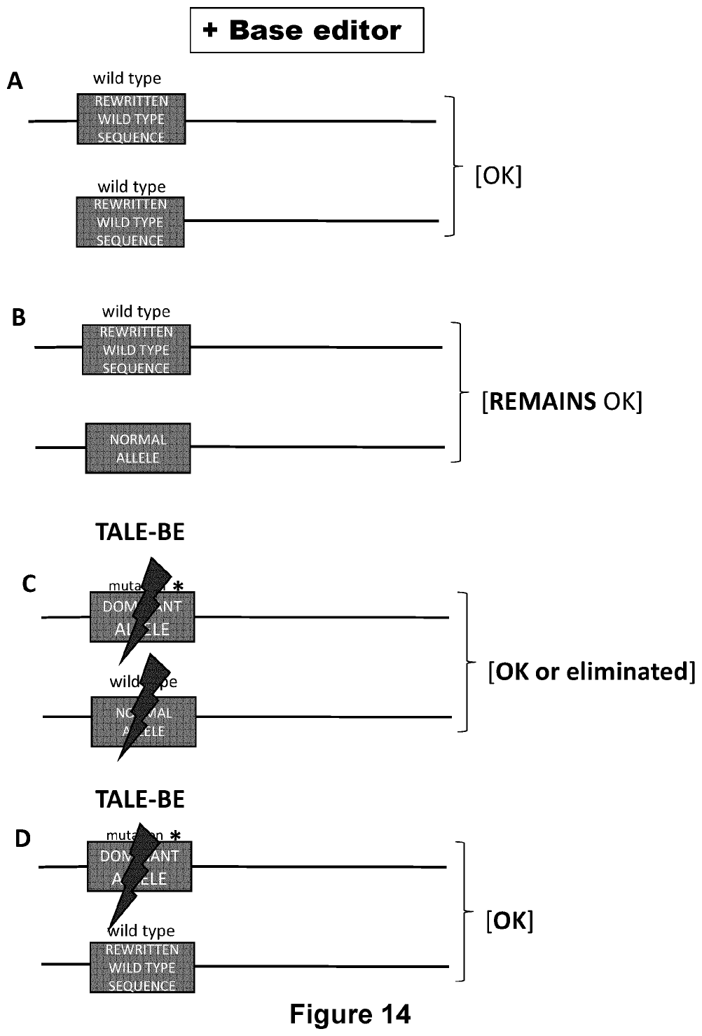

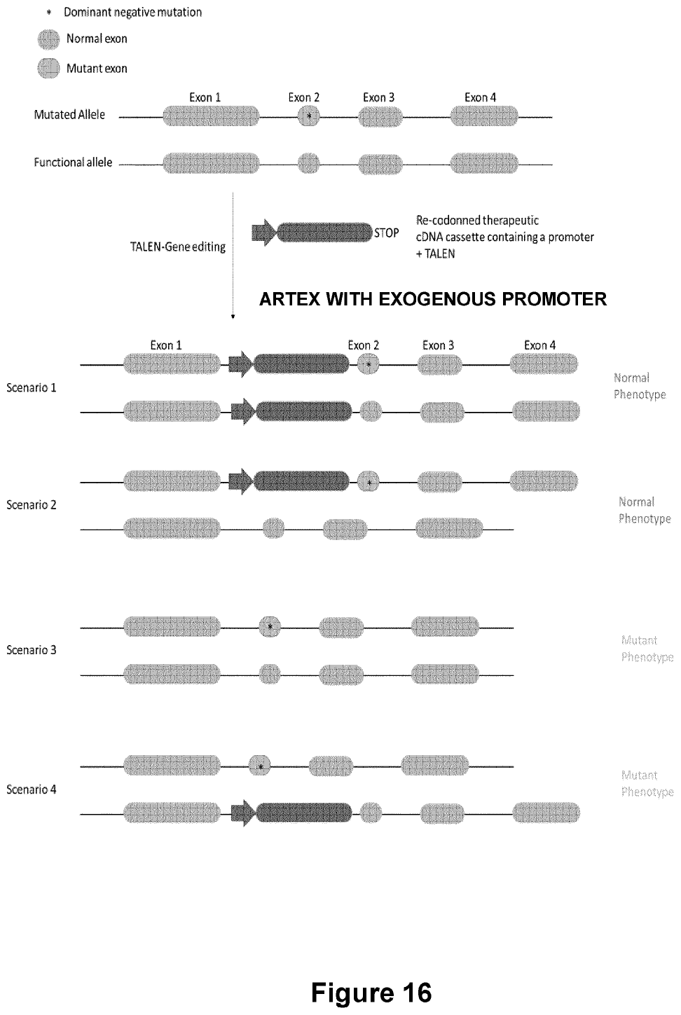

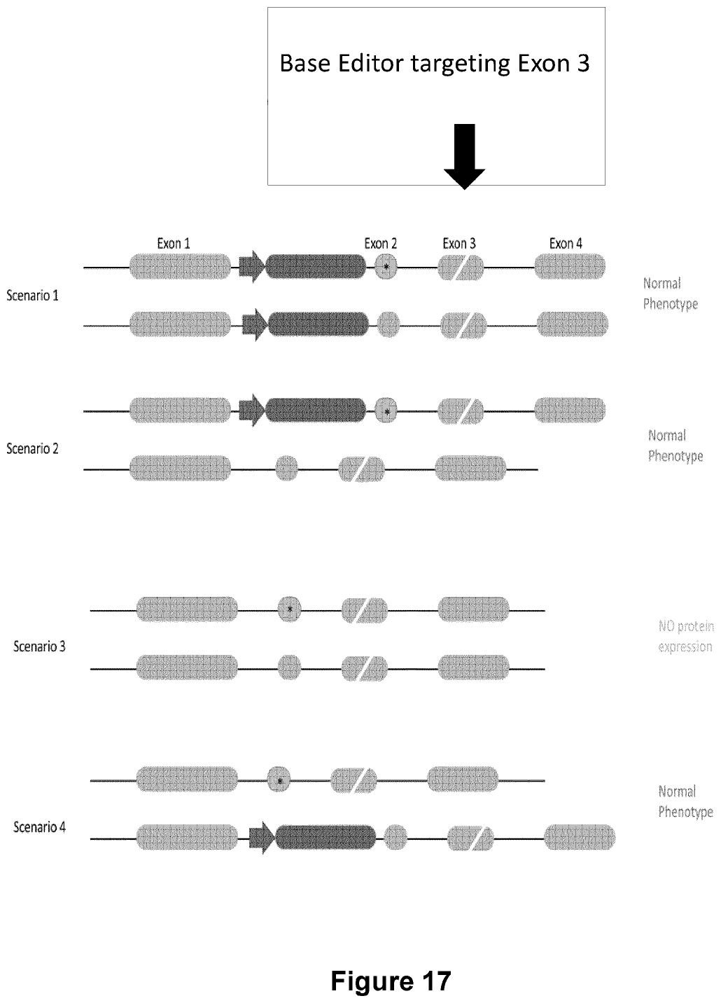

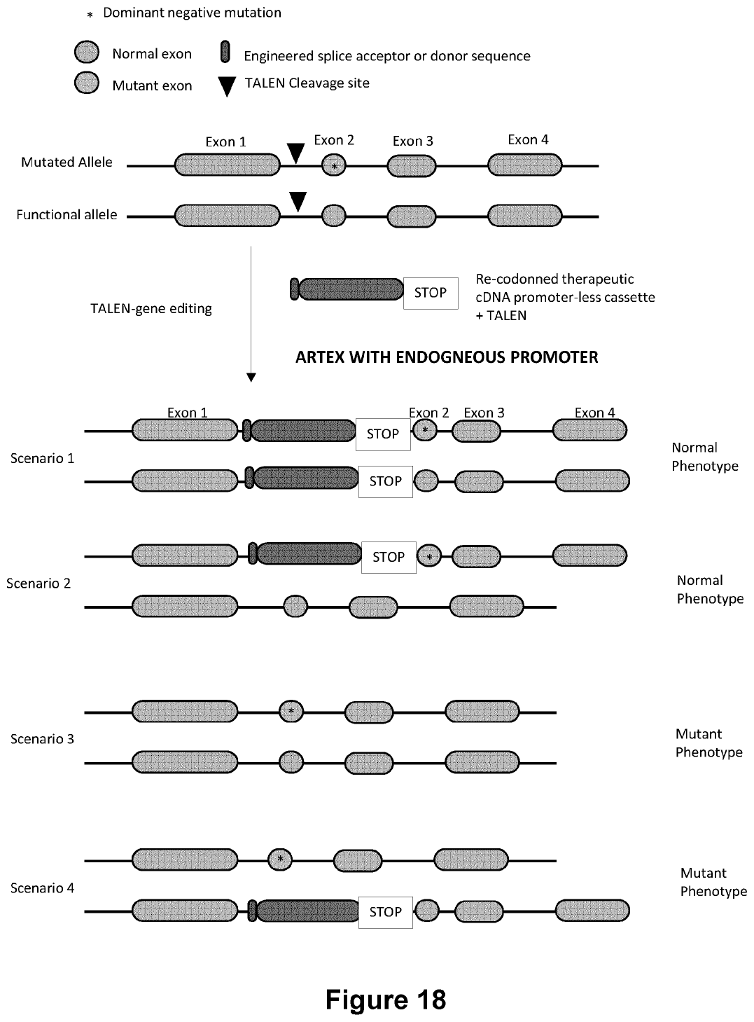

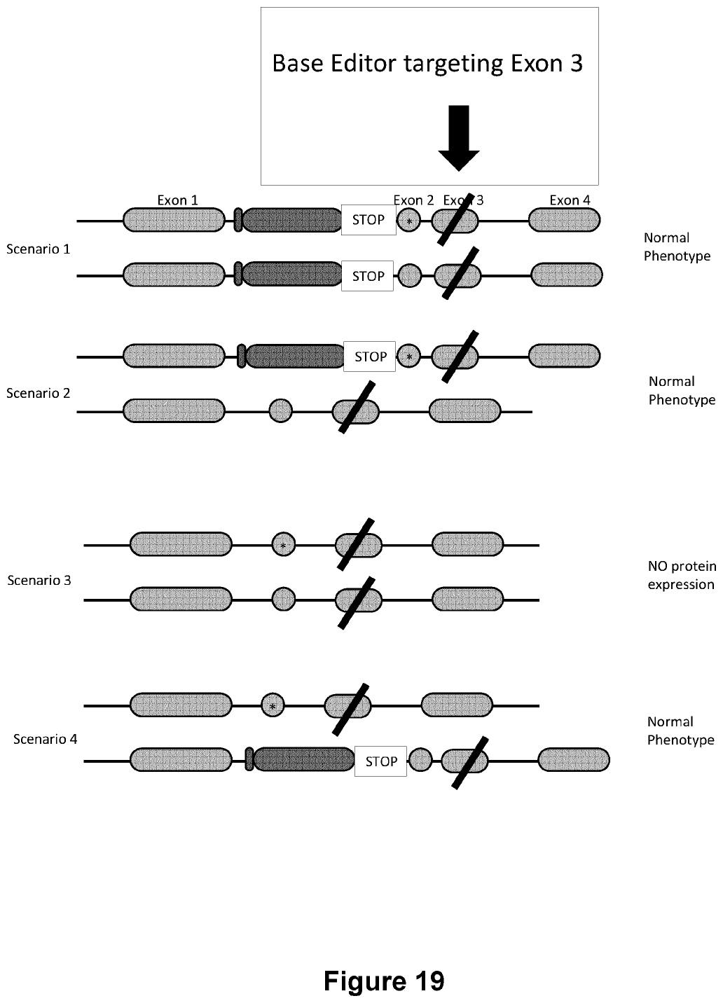

Table 19: List of exemplary disease and alleles that could be cured by the gene therapy approach as exemplary illustrated in FIGS. 12 to 17, which may consist of combining a site specific nuclease for targeted insertion of a corrected rewritten gene sequence and a sequence specific base-editor that inactivates the remaining endogenous deleterious allelic sequences.

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology [Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press; Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986].

The present invention has thus for object methods to design and produce TALE proteins to convert a specific C or its complementary G position into A/T in a double stranded nucleic acid sequence. While not always specified throughout the present document, the present teaching to target a desired C position can be straightforwardly transposed to G on the opposite DNA strand.

According to some embodiments, the method of the present invention comprises the step of identifying a target sequence into a polynucleotide sequence such as a genomic sequence, which has the following features:

5′-T₀-N_{left}-N_{y}-RTC-Nₓ-N_{right}-A₀-3′;
or
5′-T₀-N_{left}-Nₓ-GAY-N_{y}-N_{right}-A₀-3′

wherein
N can be A, T, C or G
R can be G or A, preferentially G
Y can be C or T
N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
x=2 to 6
y=6 to 10
with preferably x+y≥11, more preferably x+y=12.
   It also preferable that x is being comprised between 2 to 5, and more preferably between 3 to 5.

The inventors have also shown than TALE base editors, especially the TALEB of the present invention, were more specific towards polynucleotide target sequences represented by formula i) or ii):

i)
5′-T₀-N_{left}-N_{y}-RTCC-Nₓ-N_{right}-A₀-3′;
or
ii)
5′-T₀-N_{left}-Nₓ-GGAY-N_{y}-N_{right}-A₀-3′

and even more specific towards target sequences represented by formula iii) and iv):

iv)
5′-T₀-N_{left}-N_{y}-GTCC-Nₓ-N_{right}-A₀-3′;
or
5′-T₀-N_{left}-Nₓ-GGAC-N_{y}-N_{right}-A₀-3′

wherein
N can be A, T, C or G
R can be G or A, preferentially G
Y can be C or T
N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
   wherein x and y are preferentially defined as follows
x=2 to 4
y=6 to 8
with 11 x+y≥9, more preferably x+y=9

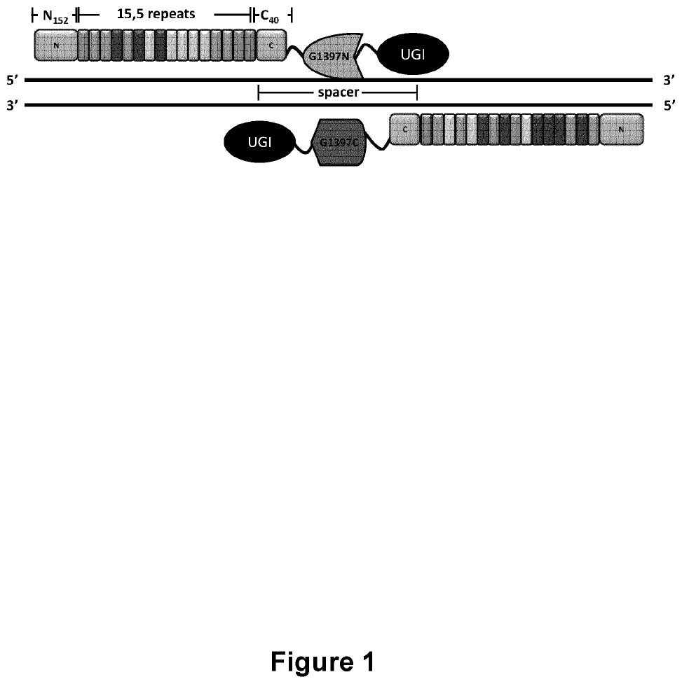

Such refined target sequences according to the present invention are useful to design and express corresponding proper specific base editor tools, in particular, by synthetizing polynucleotide sequences encoding left and right TALE binding polypeptides that respectively bind the N_{left} and N_{right} polynucleotide sequences defined above. Such polynucleotides sequences encoding left and right TALE binding polypeptides can be fused to polynucleotide sequences encoding a member of a split DddAtox to form a TALE-DddATox heterodimer, which is generally performed by fusing said member of the split DddaTox to the C terminus of said TALE binding polypeptides. The method of the invention generally further comprises the step of fusing a polynucleotide sequence encoding UGI (Uracil glycosylase inhibitor) to one monomer of said TALE-DddATox heterodimer, as illustrated in FIG. 1.

According to some embodiments, left and right TALE binding polypeptides are linked to the split DddAtox by a TALE C-terminus of 1 to 50 amino acids, preferably 8 to 40, more preferably 10 to 30, even more preferably about 40 amino acids. The invention provides with optimal scaffolds that comprise a C-terminus linker of about 11 amino acids or alternatively of about 40 amino acids, which are generally derived from the AvrBs3 original Xanthomonas TALE proteins [Christian, M. et al. TAL effector nucleases create targeted DNA double-strand breaks (2010) Genetics 186: 757-761].

By “TALE protein”, is meant herein a polypeptide that typically comprises a core DNA binding domain, which has at least 50%, preferably at least 60%, 70%, 80% or 90% identity with the DNA binding domain of wild-type AvrBs3 [also called TalC Uniprot—G7TLQ9], which represents the archetype of the family of transcription activator-like (TAL) effectors from phytopathogenic Xanthomonas campestris. Such DNA binding domain is characterized by repeated sequences of about 30 and 34 amino acids comprising variable di-residues usually found in positions 12 and 13.

By “AvrBs3-like repeats” are meant artificial arrays of about 30 to 33 amino acids, which typically comprise variable di-residues in positions 12 and 13 interacting with A, C, G or T, similarly as the above consensus AvrBs3 repeats. In other words, AvrBs3-like repeats are similar and can be combined with AvrBs3 repeats, but are generally not identical to the consensus or to the wild-type AvrBs3 repeats. It shall be noted that, in some instances, di-residues in positions 12 or 13 may be absent—so-called*(star)—to accommodate methylated bases in genomic DNA as described by [Valton et al. (2012) Overcoming Transcription Activator-like Effector (TALE) DNA Binding Domain Sensitivity to Cytosine Methylation. DNA and Chromosomes. 287(46):38427].

The AvrBs3-like repeats of the present invention generally display at least 60%, preferably at least 70%, 75%, 80%, 90% or 95% identity with either of the above AvrBs3 consensus repeats sequences of SEQ ID NO:12 to 15. They generally comprise D4 and D32 substitutions, such as in the following repeat sequences SEQ ID NO:5 to 11 of the present invention:

(SEQ ID NO: 5)
LTPDQVVAIASX₁₂X₁₃GGKQALETVQRLLPVLCQDHG,
(SEQ ID NO: 6)
LTPDQVVAIASX₁₂X₁₃GGKQALETVQALLPVLCQDHG
(SEQ ID NO: 7)
LTPDQVVAIASX₁₂X₁₃GGKQALETVQQLLPVLCQDHG,
(SEQ ID NO: 8)
LTPDQLVAIASX₁₂X₁₃GGKQALETVQRLLPVLCQDHG,
(SEQ ID NO: 9)
LTPDQMVAIASX₁₂X₁₃GGKQALETVQRLLPVLCQDHG,
(SEQ ID NO: 10)
LTPDQVVAIASX₁₂X₁₃GGKQALETVQRLLPVLCQDQG,
or
(SEQ ID NO: 11)
LTLDQVVAIASX₁₂X₁₃GGKQALETVQRLLPVLCQDHG,

wherein X₁₂X₁₃ are the di-residues interacting with a given nucleotide base pair in the targeted sequence.

The variable di-residues (X₁₂X₁₃) present in the AvrBs3-like repeats and associated with recognition of the different nucleotides are generally HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. More preferably, RVDs associated with recognition of the nucleotides C, T, A, G/A and G respectively are selected from the group consisting of NN or NK for recognizing G, HD for recognizing C, NG for recognizing T and NI for recognizing A, TL for recognizing A, VT for recognizing A or G and SW for recognizing A. More generally, RVDs associated with recognition of nucleotide C are selected from the group consisting of N*, RVDs associated with recognition of the nucleotide T are selected from the group consisting of N* and H*, where * may denote a gap in the repeat sequence that corresponds to a lack of amino acid residue at the second position of the RVD. In some embodiments, X₁₂X₁₃ can represent unusual or unconventional amino acid residues in order to modulate their specificity towards nucleotides A, T, C and G as described in Juillerat et al. [Optimized tuning of TALEN specificity using non-conventional RVDs (2015) Sci Rep 5:8150].

The AvrBs3-like repeats are generally represented by polypeptide sequences, in which X₁₂ and X₁₃ are respectively NI (to preferably target A), HD (to preferably target C), (to preferably target G) NN and NG (to preferably target T), such as in SEQ ID NO:12, 13, 14 and 15.

In some embodiments, the invention also provides a recombinant transcriptional activator-like Effector (TALE) base editor comprising one or several AvrBs3-like repeats comprising D (aspartic acid) residues at positions 4 and 32, such as in the above polynucleotide sequences SEQ ID NO:5 to 11. Such AvrBs3-like repeats can be further mutated into 1 to 5 amino acid positions, including or in addition to the D4 and D32 positions. Such recombinant transcriptional activator-like Effector (TALE) base editors can comprise one or several of such repeats to bind Nleft and Nright, to form polypeptides comprising generally from 9 to 20 repeats, preferably from 10 to 18, more preferably from 11 to 15, and alternatively from 5 to 12 repeats in situations where smaller genomes are considered, such as for instance mitochondrial genomes.

Although not mandatory, the core DNA binding domain generally comprises a half RVD made of 20 amino acids located at the C-terminus. Said core DNA binding domain thus comprises between 9.5 and 20.5 RVDs, more preferably between 10.5 and 18.5 RVDs, and even more preferably, between 11.5 and 15.5 RVDs.

As per the present invention, the core DNA binding domain as previously described, preferably comprising RVDs bearing D4 and/or D32 substitutions, is flanked by N-terminal and C-terminal sequences, said N-terminal and C-terminal sequences having preferably one of the following features detailed below.

In some embodiments, the N-terminal sequence is derived from the N-terminal domain of a naturally occurring TAL effector such as AvrBs3. In another embodiment, said additional N-terminus domain is the full-length N-terminus domain of a naturally occurring TAL effector N-terminus domain. In a further embodiment, said additional N-terminus domain is a variant which allows overcoming sequence constraints associated with the so-called “RVD0” (i.e. first cryptic repeat), such as for instance the necessity to have a T required as the first base on the binding nucleic acid sequence.

In another embodiment, said N-terminal sequence is derived from a naturally occurring TAL effector or a variant thereof. In another embodiment, said N-terminal sequence is a truncated N-terminus of such naturally occurring TAL effector or variant. In another embodiment, said additional domain is a truncated version of AvrBs3 TAL effector. In another embodiment, said truncated version lacks its N-terminal segment distal from the core TALE binding domain, such as the first 152 N-terminal amino acids residues of the wild type AvrBs3, or at least the 152 amino acids residues.

In some embodiments, the C-terminal sequence corresponds to a full or preferably truncated C-terminal region of a naturally occurring TAL effector such as AvrBs3. In general, said C-terminal sequence is a truncated version of AvrBs3 TAL effector, proximal to the core TALE binding domain, such as SEQ ID NO:2 (11 amino acids), SEQ ID NO:3 (40 amino acids), or SEQ ID NO:4 (50 amino acids) or a natural variant thereof. Accordingly, said C-terminal sequence generally comprises or consists of a polypeptide sequence having at least 85%, 90%, 95% or 99% identity with the below SEQ ID NO:2, SEQ ID NO:3 or SEQ ID NO:4:

-SEQ ID NO: 2 (C-11 AA)
SIVAQLSRPDP
-SEQ ID NO: 3 (C-40 AA):
SIVAQLSRPDPALAALTNDHLVALACLGGRPALDAVX₁X₂GL
-SEQ ID NO: 4 (C-50 AA):
SIVAQLSRPDPALAALTNDHLVALACLGGRPALDAVX₁X₂GLPHAPALI
X₃RT

In the above sequences, X₁, X₂ and X₃ represent K or an amino acid substitution introduced into the wild type AvrBs3 C-terminal polypeptide sequence, which is preferably R (arginine) or H (histidine) residue, most preferably R. X₁, X₂ and X₃ can be identical or different.

Said N-terminal sequence or C-terminal sequence can comprise a localization sequence (or signal) which allows targeting said chimeric protein toward a given organelle within an organism, a tissue or a cell. Non-limiting examples of such localization signals are nuclear localization signals, chloroplastic localization signals or mitochondrial localization signals. In another embodiment, said additional N-terminus domain can comprise a nuclear export signal having the opposite effect of a nuclear localization signal to help targeting organelles such as chloroplasts or mitochondria. In the scope of the present invention are also encompassed additional C-terminus or N-terminus sequences with a combination of several localization signals. Such combinations can be as a non-limiting example a nuclear localization signal (NLS) and/or a tissue-specific signal to help addressing said fusion protein of the present invention in the nuclear of tissue specific cells. In preferred embodiments, a NLS is generally included in the N-terminal region of the TALE-protein.

“Identity” throughout the present specification refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. The present specification generally encompasses polypeptides and polynucleotides having at least 70%, 85%, 90%, 95%, 98% or 99% identity with the specific polypeptides and polynucleotides sequences described herein, exhibiting substantially the same functions or that can be considered as equivalents.

In the present invention DddAtox refers to the wild type cytidine deaminase of SEQ ID NO:1 (Uniprot #:PODUH5) as described by Mok et al. [A bacterial cytidine deaminase toxin enables CRISPR-free mitochondrial base editing (2020) Nature. 583:631-637] derived from the microorganism Burkholderia cenocepacia, which can be split at residue 1333 or 1397 into two inactive halves referred to DddAtoxspNter (SEQ ID NO:28) and DddAtoxspCter (SEQ ID NO:29). These halves reconstitute deamination activity when assembled adjacently on target DNA driven by the TALE binding domains. In preferred embodiments, the DddAtox is split at residue 1397.

According to a preferred embodiment, which can be regarded as an invention in itself, TALE base editors specificity can be further enhanced by introducing mutations into the DddAtoxspNter (SEQ ID NO:28) and DddAtoxCter (SEQ ID NO:29) in order to lower the stability of the two split interaction. In such a way, only stronger interaction induced by TALE mediated binding between the mutated split monomers can prevail. As a result, deamination would occur at the proper targeted C position with more specificity. Mutations could be introduced at any position in SEQ ID NO:28 (DddAtoxNter split) and/or SEQ ID NO:29 (DddAtoxCter split, preferably at any position in SEQ ID NO:29 DddAtoxCter split. Also, in the methods according to the present invention, the TALE base editor monomers preferably comprise Nter and/or Cter member(s) of said split DddAtox that preferably include(s) at least one mutation or modification that decreases the affinity of the two splits DddAtox members for each other.

As another way to increase TALE base editors specificity, which may be regarded as a further invention, is a method to reduce off-target genomic mutations, wherein the polypeptide sequence of the TALE base editors heterodimer is mutated to lower its interaction with auxiliary proteins, such as CTCF (CCCTC-binding factor). CTCF is a well-known transcription factor in organizing the 3D genome architecture, which forms loop domains in a process involving the cohesin complex [Merkenschlager, M. & Nora, E. P. (2016) CTCF and Cohesin in Genome Folding and Transcriptional Gene Regulation. Annu Rev Genomics Hum Genet 17:17-43]. Recently, Lei, Z. et al. [Mitochondrial base editor induces substantial nuclear off-target mutations. Nature. (2022) doi.org/10.1038/s41586-022-04836-5] have discovered that CTCF recognition sites could bias specific TALE base editors binding to their target sites, which can result into significant off-target genome wide. It is thus anticipated that methods involving the step of selecting proper target sequences as per the present invention combined with a step of lowering the interaction of the TALE base editors with CTCF should significantly not only increase the frequency of the desire mutation but would also reduce off-target mutations within nuclear genome.

The methods of the present invention encompass the steps of expressing the polynucleotide constructs (as DNA or mRNA) described herein in cells in order to obtain their transcription and/or translation to obtain polypeptides that introduce mutations into the genome of said cells.

The present invention has also for object any polypeptide or polypeptide sequences involved in the methods described herein, especially those encoding the TALE base editors active on the genomic target sequences defined herein, as well as the cells transformed or engineered with these sequences or comprising said genomic target sequences.

Indeed, the present invention may also be regarded as a method for introducing a mutation into the genome of a cell, especially by converting C into A or G into T, comprising the step of introducing or expressing into the cell a polynucleotide encoding a TALE base editor as previously described, such as one consisting of a fusion of a left and/or right TALE binding polypeptides having a C-terminal domain of about 1 to 50 amino acids, with respectively a C terminal and/or N-terminal split DddATox. Such method preferably involves targeting a genomic sequence selected from:

5′-T₀-N_{left}-N_{y}-RTC-Nₓ-N_{right}-A₀
5′-T₀-N_{left}-Nₓ-GAY-N_{y}-N_{right}-A₀

wherein
N can be A, T, C or G
R can be G or A.
Y can be C or T
N_{left} can be a polynucleotide sequence comprising between 9 to 20 A, T, C or G;
N_{right} can be a polynucleotide sequence comprising between 9 to 20 A, T, C or G;
G being the complementary base of C.
x=2 to 6
y=6 to 10
with preferably x+y≥11, more preferably x+y=12,
wherein said heterodimeric TALE base editor binds the N_{left} and N_{right} polynucleotide sequences.

According to preferred embodiments, the left and right TALE binding polypeptides of said TALE base editors are linked to the split deaminase through a C-terminus of 1 to 50 amino acids, preferably 8 to 40, more preferably 10 to 30, even more preferably about 11 amino acids or about 40 amino acids.

According to preferred embodiments x, which determines the number of nucleotide bases into the spacer, is comprised between 2 to 5, preferably 3 to 5 to gain optimal specificity.

According to preferred embodiments, the TALE base editors of the present invention has a structure that comprises a TALE C-terminus comprising about 11 amino acids, such as SEQ ID NO: 2 or SEQ ID NO:551, this later comprising an additional GGS linker. Such TALE base editors structure is particularly suited to target sequences represented by formula i), ii), iii) and iv) as defined previously, more specifically iii) and iv) with 11≥x+y≥9, more preferably x+y=9. The present invention can be advantageously performed to introduce specific mutations in living cells, ex-vivo or in-vivo, to produce therapeutic cells, especially therapeutic immune cells.

By “immune cell” is meant a cell of hematopoietic origin functionally involved in the initiation and/or execution of innate and/or adaptative immune response, such as typically CD3 or CD4 positive cells. The immune cell according to the present invention can be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells.

In preferred embodiments the immune cells are Tumor Infiltrating Lymphocytes (TIL): TILs include, but are not limited to, CD8+ cytotoxic T cells (lymphocytes), Th1 and Th17 CD4+ T cells, natural killer cells, dendritic cells and M1 macrophages. TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient.

In preferred embodiments, the therapeutic cells are primary cells obtained from healthy donors. By “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284). The primary immune cells according to the present invention can also be differentiated from stem cells, such as cord blood stem cells, progenitor cells, bone marrow stem cells, hematopoietic stem cells (HSC) and induced pluripotent stem cells (iPS).

In preferred embodiments, the therapeutic cells of the present methods are T-cells or NK cells that may be endowed with a chimeric antigen receptor (CAR) or a recombinant TCR as described in the prior art, such as for instance into WO2013176915.

By following the teaching of the present invention, preferential safer TALE base editors target sequences in various genes have been identified for producing engineered therapeutic immune cells.

In preferred embodiments, the present methods can be used to repress or inactivate a gene encoding a component of TCR, such as one encoding TCR alpha or TCR beta, in a T-cell to produce less alloreactive T-cells that can be used in allogeneic treatment settings. More specifically, the present invention provides with a list of target window sequences into the TCRalpha (TRAC) gene (Table 7) that are particularly accessible for TALE base editors to introduce specific mutations, while reducing the risk of off-target mutations in the whole human genome.

In preferred embodiments, the present methods can be used to repress or inactivate genes, such as CD52, which code for targets of immune suppressive drugs, such as Alemtuzumab. By inactivating such genes, the therapeutic cells can become resistant to drugs that can be used in standard of care anti-cancer treatments. In other preferred embodiments, GR or DCK genes can be respectively inactivated by mutation to render the cells resistant to glucocorticoids and purine analogues.

In preferred embodiments, the methods of the invention comprises the step of introducing a TALE base editor into an immune cells that binds a genomic sequence comprised in a gene encoding a target for an immune suppressive drug such as CD52. More specifically, the present invention provides with a list of target window sequences into the CD52 gene (Table 8), especially in the splice acceptor site and signal peptide of Exon 2, that are particularly accessible for TALE base editors to introduce specific mutations, while reducing the risk of off-target mutations in the whole human genome.

In further embodiments, the methods of the invention comprises the step of introducing a TALE base editor into an immune cells that binds a genomic sequence comprised in a gene encoding an immune checkpoint protein, such as PD1, CISH, CTLA4, TIM3 or LAG3. More specifically, the present invention provides with a list of target window sequences (Table 9) into the PD1 gene that are particularly accessible for TALE base editors to introduce specific mutations, while reducing the risk of off-target mutations in the whole human genome.

In further embodiments, the methods of the invention comprises the step of introducing a TALE base editor into an immune cells that binds a genomic sequence comprised in a gene encoding beta2-microglobulin (B2M) or a human leukocyte antigen (HLA). More specifically, the present invention provides with a list of target window sequences into the B2M gene (Table 10) that are particularly accessible for TALE base editors to introduce specific mutations, while reducing the risk of off-target mutations in the whole human genome.

By target window sequences is meant a genomic sequence covered by the general formulas:

5′-T₀-N_{left}-N_{y}-RTC-Nₓ-N_{right}-A₀-3′;
or
5′-T₀-N_{left}-Nₓ-GAY-N_{y}-N_{right}-A₀- 3′

as previously defined,
   which can be spanned by one or several TALE base editors heterodimer according to the present invention taking into account x and y variations and the number of nucleotides comprised into N_{left} and N_{right} sequences.

Further examples of mutations into immune checkpoint genes and genes are provided in the literature and especially in WO2019016360 to produce different attributes of therapeutic engineered immune cells.

According to preferred embodiments, the present methods combine the use of TALE base editors and rare-cutting endonucleases, especially TALE-nuclease, for multiplexing gene editing in immune cells.

In some embodiments, the TALE base editors and rare-cutting endonucleases can be co-expressed, concomitantly transfected or sequentially introduced by minimizing the risk of chromosomal defects.

As shown for instance in Example 4, particular combinations have resulted into extremely low levels of translocations, off-sites and/or chromosomal rearrangements:
inactivation of TCR using a rare-cutting endonuclease and introducing a or several point mutations into the CD52 gene by using TALE base editors;
inactivation of TCR using a rare-cutting endonuclease and introducing a or several point mutations into TGFBRII gene by using TALE base editors;
inactivation of immune checkpoint gene, such as PD1, CISH, CTLA4, TIM3 or LAG3 using a rare-cutting endonuclease and introducing a or several point mutations into TCR, by using TALE base editors;
inactivation of TCR using a rare-cutting endonuclease and introducing a or several point mutations into an immune checkpoint gene, such as PD1, CISH, CTLA4, TIM3 or LAG3, by using TALE base editors;
inactivation of TCR using a rare-cutting endonuclease and introducing a or several point mutations into a gene component of MHC, such as HLA-A, HLA-B, HLA-C or B2M by using TALE base editors;
inactivation of gene component(s) of MHC, such as HLA-A, HLA-B, HLA-C or B2M using a rare-cutting endonuclease and introducing a or several point mutations into TCR by using TALE base editors;

This combination approach, which is an important part of the invention, is particularly useful to combine knock-in (ex: targeted gene insertion) and/or knock-out (ex: gene inactivation) multiplexing in immune cells. In particular, rare-cutting endonucleases can be used to introduce an exogenous polynucleotide sequence in the genome at a first locus by site directed gene integration, while a TALE base editors can be concomitantly used to introduce a or several point mutations at another locus, especially a locus that needs to be inactivated.

For instance:
a rare-cutting endonuclease can be used to inactivate B2M expression and to introduce at this locus an exogenous polynucleotide sequence encoding HLAE to make the cell invisible to NK cells, whereas in the meantime, a TALE base editors can be used to introduce a or several point mutations as previously proposed into TCR and/or CD52;
a rare-cutting endonuclease can be used to inactivate an immune checkpoint gene, such as PD1, CISH, CTLA4, TIM3 or LAG3, and introduce at such locus an exogenous polynucleotide sequence encoding a chimeric antigen receptor (CAR), whereas in the meantime, a TALE base editors can be used to introduce a or several point mutations as previously proposed into TCR and/or CD52;
a rare-cutting endonuclease can be used to inactivate an immune checkpoint gene, such as PD1, CISH, CTLA4, TIM3 or LAG3, and to introduce at such locus an exogenous polynucleotide sequence encoding a cytokine, such as IL-2, IL-12, IL-18 . . . , whereas in the meantime, a TALE base editors can be used to introduce a or several point mutations as previously proposed into TCR and/or CD52.
a rare-cutting endonuclease can be used to inactivate the expression of a component of TCR, such as TRAC, and to introduce at such locus an exogenous polynucleotide sequence encoding a CAR or a recombinant TCR, whereas in the meantime, a TALE base editors can be used to introduce a or several point mutations as previously proposed into an immune checkpoint and/or CD52.

As shown in the examples, the above embodiments combining knock-out and targeted gene insertion, such as by using an AAV vector comprising a transgene, for instance to introduce said transgene by homologous recombination (HDR), prevent incidental transgene trapping (more specifically referred to as “AAV trapping”) when the genome is concurrently knocked out at another locus. In this manner, nucleases can be used for gene insertion, while TALE base editors are concurrently used to inactivate gene(s) located at other locations in the genome.

By “rare-cutting endonucleases” is meant sequence-specific endonuclease reagent that is not naturally found in mammalian cells, which recognition sequences generally range from 10 to 50 successive base pairs, preferably from 12 to 30 bp, and more preferably from 14 to 20 bp. Such endonuclease reagent is generally a nucleic acid encoding an “engineered” or “programmable” rare-cutting endonuclease, such as a homing endonuclease as described for instance by Arnould S., et al. [WO2004067736], a zinc finger nuclease (ZFN) as described, for instance, by Urnov F., et al. [Highly efficient endogenous human gene correction using designed zinc-finger nucleases (2005) Nature 435:646-651], a TALE-Nuclease as described, for instance, by Mussolino et al. [A novel TALE nuclease scaffold enables high genome editing activity in combination with low toxicity (2011) Nucl. Acids Res. 39(21):9283-9293], or a MegaTAL nuclease as described, for instance by Boissel et al. [MegaTALs: a rare-cleaving nuclease architecture for therapeutic genome engineering (2013) Nucleic Acids Research 42(4):2591-2601]. Due to their higher specificity, TALE-nuclease have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. [TALEN facilitate targeted genome editing in human cells with high specificity and low cytotoxicity (2014) Nucl. Acids Res. 42(10): 6762-6773]. RNA-guides to be used in conjunction with a RNA guided endonuclease, such as Cas9 or Cpf1, as per, inter alia, the teaching by Doudna, J., and Chapentier, E., [The new frontier of genome engineering with CRISPR-Cas9 (2014) Science 346 (6213):1077] are also rare-cutting endonucleases contemplated by the present invention.

According to a preferred aspect of the invention, the endonuclease reagent is transiently expressed into the cells, such as be the case of RNA, more particularly mRNA, proteins or complexes mixing proteins and nucleic acids conjugates involving polynucleotide(s) and polypeptide(s) such as so-called “ribonucleoproteins”. Such conjugates can be formed more particularly with reagents as Cas9 or Cpf1 (RNA-guided endonucleases) with their RNA-guides as described for instance by Zetsche, B. et al. [Cpf1 Is a Single RNA-Guided Endonuclease of a Class 2 CRISPR-Cas System (2015) Cell 163(3): 759-771].

In general, electroporation steps are used to transfect the immune cells with either or both the nucleases and the TALE base editors, which is typically performed in closed chambers comprising parallel plate electrodes producing a pulse electric field between said parallel plate electrodes greater than 100 volts/cm and less than 5,000 volts/cm, substantially uniform throughout the treatment volume such as described in WO2004083379, which is incorporated by reference, especially from page 23, line 25 to page 29, line 11. One such electroporation chamber preferably has a geometric factor (cm-1) defined by the quotient of the electrode gap squared (cm2) divided by the chamber volume (cm3), wherein the geometric factor is less than or equal to 0.1 cm-1, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform. Multiplexing of rare-cutting endonuclease and TALE base editors in immune cells can be performed by following the protocol previously reported with respect to nucleases [Poirot et al. (2013) Blood. 122 (21): 1661 and Sachdeva et al. (2019) Nat Commun. 10 (1)].

“Exogenous sequence” refers to any nucleotide or nucleic acid sequence that was not initially present at the selected locus. This sequence may be homologous to, or a copy of, a genomic sequence, or be a foreign sequence introduced into the cell. The exogenous sequence preferably codes for a polypeptide which expression confers a therapeutic advantage over sister cells that have not integrated this exogenous sequence at the locus. The exogenous sequence is generally introduced into the cell as a donor template and integrated into the genome by homologous recombination induced by the rare-cutting endonuclease. This donor template can be introduced into the cell by transduction under the form of a viral vector, such as an AAV, or can be introduced as a polynucleotide such as single stranded oligonucleotides (ssODNs) as described for instance WO2021224395.

The present methods can result into immune cells comprising and/or co-expressing a rare-cutting endonuclease and a TALE base editors as described herein, as populations of cells or intermediary product cells for producing engineered therapeutic cells or cell compositions.

According to some aspects of the invention, the present TALE base editors are used in gene therapy for in-vivo gene correction or the inactivation of deficient gene expression. In particular, the TALE base editors as per the present invention can be directed towards liver cells in-vivo to target viral genomes, such as the cccDNA (covalently closed circular DNA) of Hepadnavirus, in particular HBV (Hepatitis B Virus), which are resistant forms of these viruses lodged into hepatocytes.

Encapsulation of mRNA or polypeptides into nanocarriers, such as liposomes, polymers, and inorganic nanoparticles, have already shown great potential for delivery of gene editing reagents into hepatocytes [Witzigmann, D. et al. (2020) Lipid nanoparticle technology for therapeutic gene regulation in the liver. Advanced Drug Delivery Reviews, 159: 344-363].

Various types of biodegradable delivery capsules comprising under the form of RNA reagents can be manufactured, depending on the structure of the biodegradable matrices involved and the monomers forming said core hydrophobic domain and polar domains. Delivery specificity can be improved by linking a targeting domain to the proximal polar domain of said nanocarriers, such that the delivery capsules can bind surface antigens of different cell types. The delivery capsules are particularly suited for intravenous injection to target endogenous genetic sequences into cells. Such delivery capsules according to the invention are useful to deliver TALE base editors into the cells under RNA form, especially the co-delivery of messenger RNAs encoding right and left heterodimers TALE base editors.

The present application more particularly claims pharmaceutical compositions comprising the biodegradable delivery capsules of the invention into treatments involving the TALE base editors as per the invention. Such treatments may be part of a gene therapy, where specific genetic sequences have to be knocked-out or repaired, of an anti-infection therapy, by targeting the genome of infectious agents, or inherited deficient genes, such as ApoC3, Transthyretin (TTR) ANGPTL3 and PCSK9 genes, which are respectively useful for treating or preventing Atherosclerosis, Transthyretin (TTR)-mediated amyloidosis (ATTR), hyperlipidemia and hypercholesterolemia.

In preferred embodiments, the present invention provides with a list of target window sequences into the ApoC3 gene (Table 11), that are particularly accessible for TALE base editors to introduce specific mutations, while reducing the risk of off-target mutations in the whole human genome.

According to more specific embodiments, the present invention provides with methods to introduce mutations into TRAC, CD52, PD1, B2m and ApoC3 by targeting any of the target sequences presented into Tables 7 to 11 respectively by using TALE base editors as described herein.

In particular, the present invention includes methods wherein a TALE base editor binds a genomic sequence comprised in a gene encoding TRAC selected from any one of SEQ ID NO:366 to SEQ ID NO:407 as indicated in Table 7.

In particular, the present invention includes methods wherein a TALE base editor binds a genomic sequence comprised in a gene encoding CD52 selected from any one of SEQ ID NO:408 to SEQ ID NO:422 as indicated in Table 8.

In particular, the present invention includes methods wherein a TALE base editor binds a genomic sequence comprised in a gene encoding PD1 selected from any one of SEQ ID NO:423 to SEQ ID NO:466 as indicated in Table 9.

In particular, the present invention includes methods wherein a TALE base editor binds a genomic sequence comprised in a gene encoding B2m selected from any one of NO:467, SEQ ID NO:501 as indicated in Table 10.

In particular, the present invention includes methods wherein a TALE base editor binds a genomic sequence comprised in a gene encoding ApoC3 selected from any one of SEQ ID NO:502 and SEQ ID NO:523 as indicated in Table 11.

According to a further aspect of the invention, mutation(s) can be induced by the TALE base editors directly into a RNA transcript within the cell. This RNA editing method combines the introduction into the cell of a single stranded DNA, such as ssODN, and a heterodimeric TALE base editors as described herein, wherein said target RNA transcript is hybridized with single stranded DNA to form a double stranded nucleic acid which is bound by said heterodimeric TALE base editors, resulting into a mutation being introduced at the desired C (or G) position in the target sequence directly at the transcript level.

As per a further embodiment of the present invention is a method to correct genetic deficiencies, in particular dysfunctional dominant alleles, by combining targeted gene integration, such as one resulting from homologous recombination, and inactivation of a endogenous gene by a sequence specific base editor, such as a TALE-base editor as previously described herein. Principle and schematic representations are illustrated in FIGS. 12 to 17 herein provided as examples. Such a gene therapy method may consist in using a sequence specific nuclease to insert a functional copy of a gene or a part thereof, or a corrected sequence thereof, in combination with the introduction into the cell of a sequence specific base editor reagent that is used to inactivate the residual endogenous sequences that have not been replaced or corrected. In some instances, the corrected sequence that is integrated at the endogenous locus has been rewritten with respect to the original endogenous sequence by using alternative codons. The sequence specific base editor that recognizes the remaining intact endogenous allele sequence, preferably one deficient that causes genetic disease, can be introduced in the cell by different means known by one skilled in the art, such as a purified protein, mRNA or viral or non-viral expression vector.

According to preferred embodiments, the gene therapy involves a site-specific endonuclease, such as a TALE-nuclease, Zinc finger nuclease, meganuclease or RNA-guided endonuclease to perform targeted gene integration in combination with a sequence specific base editor such as a TALE base editors previously described. The site-specific endonuclease is co-transfected with a DNA template, such as a AAV vector or single stranded DNA, encoding a functional allele sequence, designed to promote its integration by homologous recombination.

According to preferred embodiments, said site-specific endonuclease and sequence specific base editor are introduced sequentially into the cell or concomitantly, such as for instance by co-transfection. Co-transfection by electroporation of mRNA encoding both reagents is preferred, but other technical solutions are possible, such as combining viral vectorization, electroporation, nanoparticles, ribonucleotide or purified protein transfection.

According to preferred embodiments the introduction of the site-specific endonuclease and the sequence specific base editor is performed ex-vivo, such as in blood immune cells, preferably primary immune cells, such as in HSCs or progeny thereof.

According to preferred embodiments, the sequence which is integrated in the genome aiming at correcting the genetic deficiency is “rewritten”, meaning that an alternative genetic code is used, in general through alternative codon usage, different from that of the endogenous allele. Thereby, the integrated rewritten sequence is not recognized by the sequence specific base editor that is directed against the corresponding endogenous allele sequence(s).

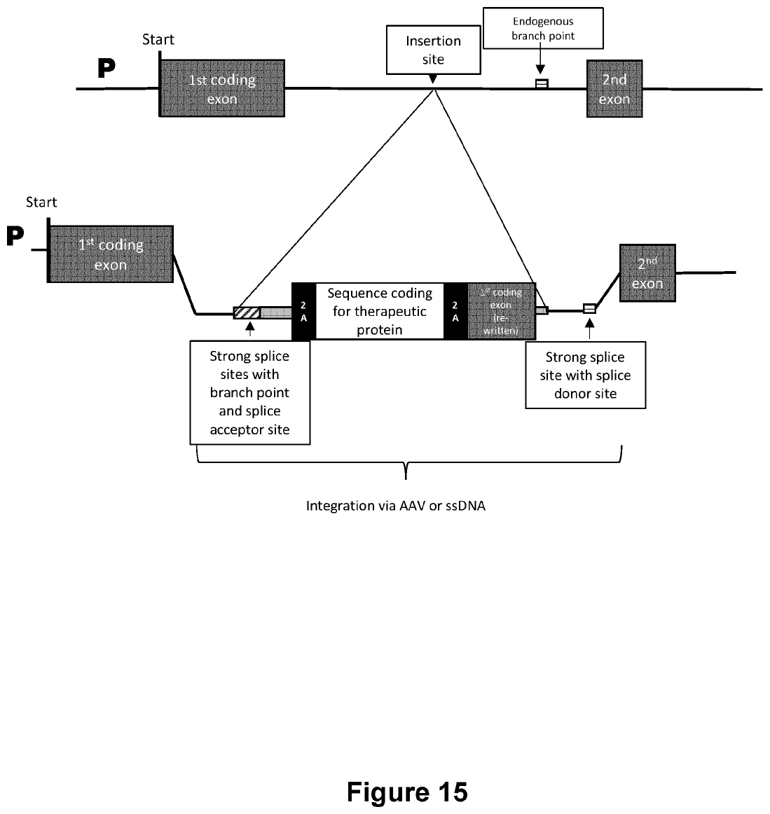

According to preferred embodiments, the functional gene sequence aiming to correct the genetic deficiency may be that of an exon or a part thereof, which can be introduced in the genome for instance as per the strategy “Artex” described in FIG. 15 and in WO2021224416, incorporated by reference.

According to preferred embodiments, the gene therapy methods of the present invention target a dysfunctional allele causing a disease selected from one listed in Table 19.

Variation of the above methods can also be considered to improve its efficiency by changing different parameters, such as one of the following:
The therapeutic integrated sequence may be inserted at any preferred locus in the genome, not necessarily at the locus of the deficient allele.
The therapeutic integrated sequence can be promoterless and inserted upstream the mutation associated with the disease. In such instances, the base editor used is preferably designed to edit the exon downstream the therapeutic insertion.
Multiple sequence specific base editors targeting different exons of one faulty gene can be involved.

The present invention is thus drawn to a therapeutic method comprising one or several of the steps comprising:
Introducing and/or expressing a transgene in a cell inserted at an endogenous locus to correct a genetic deficiency,
Introducing and/or expressing in said cell, a sequence specific base editor that target the allelic endogenous sequence(s) causing said genetic deficiency to inactivate its expression.

The above steps can take place simultaneously or sequentially. The introduction of the transgene can be performed by different means known in the art, viral or non-viral, such as by introducing a DNA template encoding said transgene in combination with a site specific rare-cutting endonuclease.

It is an advantage of the present method to combine site-specific nuclease and base editors because they can be concomitantly introduced in the cells, such as by electroporation without the risk of interacting one with the other. By contrast to using multiple nucleases that may create chromosomal deletions or rearrangement, the combined and concomitant use of site-specific nuclease and sequence specific base editors, especially TALE nucleases and TALE base editors, is deemed safe and without known negative interactions.

The present gene therapy methods are not limited to the combined use of TALE base editors and TALE-nucleases as described in the examples, and can be carried out using other site-specific endonuclease reagents, such as RNA guided endonucleases (ex: Cas9, Cas12 . . . ), and other kind of sequence-specific base editors, such as those composed by a catalytically dead Cas9 (dCas9) or a nickase Cas9 (nCas9) fused to a deaminase and guided by a single guide RNA (sgRNA) to the locus of interest, and any combinations thereof.

Preferably, the transgene sequence has a rewritten or distinct genetic sequence with respect to the endogenous allele causing the genetic deficiency, such that the sequence specific base editor can easily discriminate the endogenous deficient allele and the transgene that correct the genetic deficiency.

In some embodiments, one or several of the following steps can be carried out sequentially or concomitantly:
Introducing or expressing a rare-cutting endonuclease targeting an endogenous locus into a cell that comprises a deficient gene sequence causing a genetic deficiency,
Introducing into said cell a DNA template to correct that genetic deficiency by gene integration at the endogenous locus targeted by said rare-cutting endonuclease,
Introducing or expressing a base editor, preferably a TALE base editors such as one described herein, to inactivate at least one endogenous allele causing said genetic deficiency.

The above methods are particularly adapted for genetic deficiencies caused by a dominant allele as they concur to inactivate all alleles putatively involved in the genetic deficiency, while providing exogenous functional copies of such alleles. A non-limited list of such genetic deficiencies is provided in Table 19. The methods of the present invention appear to be particularly suited for engineering curative HSCs or T-cells ex-vivo in view of being administered to patients for treating a genetic deficiency, in particular for treating ADPS1 and STAT3.

One aspect of the invention are the engineered curative cells obtainable and/or involved in the above gene therapy methods, such as HSCs or progeny thereof, which typically comprise a transgene to correct a genetic deficiency, said transgene being generally a corrected and/or rewritten version of a deficient endogenous allele causing said genetic deficiency, wherein the endogenous alleles causing said genetic deficiency have been inactivated (mutated) by at least one base editor.

Such engineered curative cells obtainable and/or involved in the above gene therapy methods, such as HSCs or progeny thereof, can typically comprise (1) a transgene to correct a genetic deficiency, said transgene being generally a corrected and/or rewritten version of a deficient endogenous allele causing said genetic deficiency, and (2) a base editor or a transgene sequence encoding same to inactivate the endogenous allele causing the genetic deficiency, and optionally, (3) a rare-cutting endonuclease or a transgene sequence encoding same to integrate said transgene at a selected endogenous locus.

Having generally described this invention, a further understanding can be obtained by reference to certain specific examples, which are provided herein for purposes of illustration only, and are not intended to limit the scope of the claimed invention.

### EXAMPLES

### Example 1

### Materials and Methods

### T Cell Culture

Cryopreserved human PBMCs were acquired from ALLCELLS. PBMCs were cultured in X-vivo-15 media (Lonza Group), containing 20 ng/ml human IL-2 (Miltenyi Biotec), and 5% human serum AB (Seralab). Human T cell activator TransAct (Miltenyi Biotec) was used to activate T cells at 25 μl TransAct per million CD3+ cells the day after thawing the PBMCs. TransAct was kept in the culture media for 72 hours.

### TALE-Nuclease and TALEB Production

TALEN (fusion TALE Nter (delta152)-repeats15,5-Cter(40)-Fok1 nuclease domain) and TALE-base editors (Left TALE binding domain Nter (delta152)-repeats15,5-Cter(40)-DddAtoxsp-Nter-UGI and Right TALE binding domain Nter(delta152)-repeats15,5-Cter(40)-DddAtoxsp-Cter) heterodimers as illustrated in FIG. 1 were assembled using standard molecular biology and/or microbiology technics such as enzymatic restriction digestion, ligation, bacterial transformation and plasmid DNA extraction (NEB 10-beta competent E. coli for ccdB selection or NEB stable competent E. coli for blue/white screening) and plasmid DNA extraction. TALE DNA targeting array were assembled and cloned in respective TALEN backbones (pCLS32783) and/or TALE base editors backbones (pCLS35714 and pCLS35715).

Small Scale mRNA Production

Plasmids of the 37 TALE base editors and 37 matching TALE-Nuclease derived from the above backbones, containing a T7 promoter and a polyA sequence, were produced as non-clonal after assembly (transformant was directly inoculated for culture and plasmid preparation). The plasmids were then linearized with SapI (NEB) and mRNA was produced by in vitro transcription (NEB HiScribe ARCA, NEB).

### Small Scall TALE-Nuclease and TALE Base Editors Testing (37 Endogenous Targets and TRAC/CD52 Multiplex Engineering)

T cells activated with TransAct (Miltenyi Biotec) for 3 days were transferred into fresh complete media containing 20 ng/ml human IL-2 (Miltenyi Biotec), and 5% human serum AB (Seralab) 10-12 hrs before transfection. Harvested cells were washed once with warm PBS. 1E6 PBS washed cells were pelleted and resuspended in 20 μl Lonza P3 primary cell buffer (Lonza). 1 μg/arm/million cells of mRNA for TALE-Nuclease or TALE base editors was mixed with the cells and then the cell mixture was electroporated using the Lonza 4D-Nucleofector under the E0115 program for stimulated human T cells. After electroporation, 80 μl warm complete media was added to the cuvette to dilute the electroporation buffer, the mixture was then carefully transferred to 400 ml pre-warmed complete media in 48-well plates. TALE-Nuclease transfected cells were incubated at 30° C. for an overnight culture and then transferred back to 37° C. incubator. TALE base editors transfected cells were incubated at 37° C. throughout the process. Cells were harvested at Day 6 post transfection for gDNA extraction and NGS analysis.

Large Scale TALE-Nuclease and TALEB mRNA Production (CD52 Targeting Base Editors)

Plasmids encoding the TRAC TALE-Nuclease contained a T7 promoter and a polyA sequence. The TALE-Nuclease mRNA from the TRAC TALE-Nuclease plasmid was produced by Trilink. Sequence targeted by the TRAC TALE-Nuclease (17-bp recognition sites, upper case letters, separated by a 15-bp spacer):

(SEQ ID NO: 31)
5′-TTCCTCCTACTCACCATcagcctcctggttatGGTACAGGTAAGA
GCAA-3′

The TALE-Nuclease mRNA from the CD52 TALE-Nuclease plasmid was produced by Trilink. Sequence targeted by the CD52 TALE-Nuclease (17-bp recognition sites, upper case letters, separated by a 15-bp spacer):

(SEQ ID NO: 32)
5′-TTCCTCCTACTCACCATcagcctcctggttatGGTACAGGTAAGA
GCAACGCCTGGCA-3′

Plasmids encoding TALE base editors T-25 and CD52 TALE base editors contained a T7 promoter and a polyA sequence. Sequence verified plasmids were linearized with SapI (NEB) before in vitro mRNA synthesis. mRNA was produced with NEB HiScribe™ T7 Quick High Yield RNA Synthesis Kit (NEB). The 5′capping reaction was performed with ScriptCap™ m7G Capping System (Cellscript). Antarctic Phosphatase (NEB) was used to treat the capped mRNA and the final cleanups was performed with Mag-Bind TotalPure NGS beads (Omega bio-tek) and Invitrogen DynaMag-2 Magnet (ThermoFisher).

ssODN Repair Template Transfection

The ssODN pool targeting the TRAC locus (SEQ ID NO: 33 to 69; see table 1) were ordered from Integrated DNA Technologies (IDT) and resuspended in ddH2O at 50 pmol/μl.

T cells activated with TransACT for 3 days were transferred into fresh complete media containing 20 ng/ml human IL-2 (Miltenyi Biotec), and 5% human serum AB (Seralab) 10-12 hrs before transfection.

The harvested cells were washed once with warm PBS. 1E6 PBS washed cells were pelleted and resuspended in 20 μl Lonza P3 primary cell buffer (Lonza). 200 pmol ssODN pool and 1 μg/arm of TRAC TALE-Nuclease were mixed with the cell and then the cell mixture was electroporated using the Lonza 4D-Nucleofector under the E0115 program for stimulated human T cells. After electroporation, 80 μl warm complete media was added to the cuvette to dilute the electroporation buffer, the mixture was then carefully transferred to 400 ml pre-warmed complete media in 48-well plates. Cells transfected with ssODN and TALE-Nuclease were then incubated at 30° C. until 24 hrs post TALE-Nuclease transfection before transfer back to 37° C.

Cells with ssODN KI were cultured for two days before harvesting for TALE base editors treatment. The harvested cells were washed once with warm PBS. 1E6 PBS washed cells were pelleted and resuspended in 20 μl Lonza P3 primary cell buffer (Lonza). 1 μg/arm of TALE base editors T-25 were mixed with the cell and then the cell mixture was electroporated using the Lonza 4D-Nucleofector under the E0115 program for stimulated human T cells. After electroporation, 80 μl warm complete media was added to the cuvette to dilute the electroporation buffer, the mixture was then carefully transferred to 400 ml pre-warmed complete media in 48-well plates. Cells transfected with TALE base editors incubated at 37° C. for 2 more days before harvesting for gDNA extraction and NGS analysis.

### Large Scale CD52 TALE Base Editors Testing

T cells activated with TransACT for 3 days were transferred into fresh complete media containing 20 ng/ml human IL-2 (Miltenyi Biotec), and 5% human serum AB (Seralab) 10-12 hrs before transfection.

The harvested cells were washed twice with Cytoporation Media T (BTXpress, 47-0002). 5E6 washed cells were pelleted and resuspended in 180 μl Cytoporation Media T. 2 μg/arm/million cells of TALE base editors mRNA was mixed with the cells to a final volume of 200 μl and then the cell/mRNA mixture was electroporated using the BTX Pulse Agile in 0.4 cm gap cuvettes. After electroporation, 180 μl warm complete media was added to the cuvette to dilute the electroporation buffer, and the mixture was then carefully transferred to 2 ml pre-warmed complete media in 12-well plates. TALE base editors transfected cells were incubated at 37° C. throughout the process. Cells were harvested at Day 6 post transfection for gDNA extraction and NGS analysis.

### Genomic DNA Extraction

Cells were harvested and washed once with PBS. Genomic DNA extraction was performed using Mag-Bind Blood & Tissue DNA HDQ kits (Omega Bio-Tek) following the manufacturer's instructions

### Targeted PCR and NGS

100 μg genomic DNA was used per reaction in a 50 μl reaction with Phusion High-Fidelity PCR Master Mix (NEB). The PCR condition was set to 1 cycle of 30 s at 98° C.; 30 cycles of 10 s at 98° C., 30 s at 60° C., 30 s at 72° C.; 1 cycle of 5 min at 72° C.; hold at 4° C. The PCR product was then purified with Omega NGS beads (1:1.2 ratio) and eluted into 30 μl of 10 mM Tris buffer pH7.4. The second PCR which incorporates NGS indices was then performed on the purified product from the first PCR. 15 ul of the first PCR product were set in a 50 μl reaction with Phusion High-Fidelity PCR Master Mix (NEB). The PCR condition was set to 1 cycle of 30 s at 98° C.; 8 cycles of 10 s at 98° C., 30 s at 62° C., 30 s at 72° C.; 1 cycle of 5 min at 72° C.; hold at 4° C. Purified PCR products were sequenced on MiSeq (Illumina) on a 2×250 nano V2 cartridge.

### Flow Cytometry

TRAC KO was monitored using an anti-TCRa/b antibody (Biolegend, #306732, clone IP26, BV605). CD52 KO was monitored using an anti-52 antibody (BD Biosciences, #563609, Clone 4C8, AlexaFlour488). Flow cytometry was performed on BD FACSCanto (BD Biosciences) and data analysis processed with FlowJo. Cell population was first gated for lymphocytes (SSC-A vs. FSC-A) and singlets (FSC-H vs. FSC-A). The lymphocyte gate was further analyzed for expression of CD52 and -TCRa/b expression from this gated population.

### In Silico Off-Site Prediction

To evaluate possible off-target editing of the CD52 TALE base editors, we generated in silico a list of potential off site targets of these base editors. That list was generated as follow. The TALE base editors have two binding sequences of 17 bp separated by a spacer. These binding sequences begin necessarily by a T. Hence, we first selected as potential targets all genomic sequences starting with a T, ending with an A, and having a size comprised between 27 bp and 67 bp (both included), allowing for spacers ranging from 10 to 40 bp). Then, the number of mismatches between the binding sequences of the potential target versus the actual TALE base editors target was counted. If that total number was greater than 8, the potential target was removed. Finally, all potential targets lacking a G in the left half of the spacer, or a C in the right half of the spacer (editing windows) were discarded.

Off-Site and Translocation Multiplexed Amplicon Sequencing rhAmp primers were designed on the on-target and/or off-target sites established by an in silico off-site prediction. Locus-specific forward and reverse primers were obtained from Integrated DNA Technologies (IDT) either in ready to use pools or individually plated, and use accordingly to IDT protocol for RNase H2-dependent multiplex assay amplification (1 cycle of s at 95° C. 10 min; 14 cycles of 15 s at 95° C. followed by 8 min at 65° C.; 1 cycle of 15 min at 99.5° C.; hold at 4° C.) followed by a universal PCR to add indexes (i5 or i7) for NGS (1 cycle of s at 95° C. 3 min; 24 cycles of 15 s at 95° C. followed by 30 s at 60° C. and 30 s at 72° C.; 1 cycle of 1 min at 72° C.; hold at 4° C.). Purified PCR amplicons were sequenced on a NextSeq (Illumina) on a NextSeq 500/550 Mid Output Kit (150 cycles) cartridge.

### Example 2: TALE-Nuclease and TALEB Efficiency Comparison

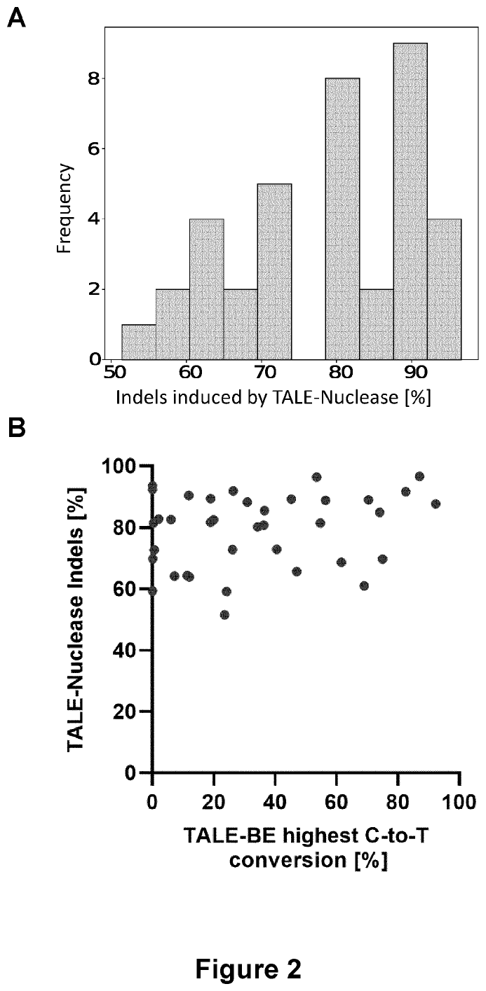

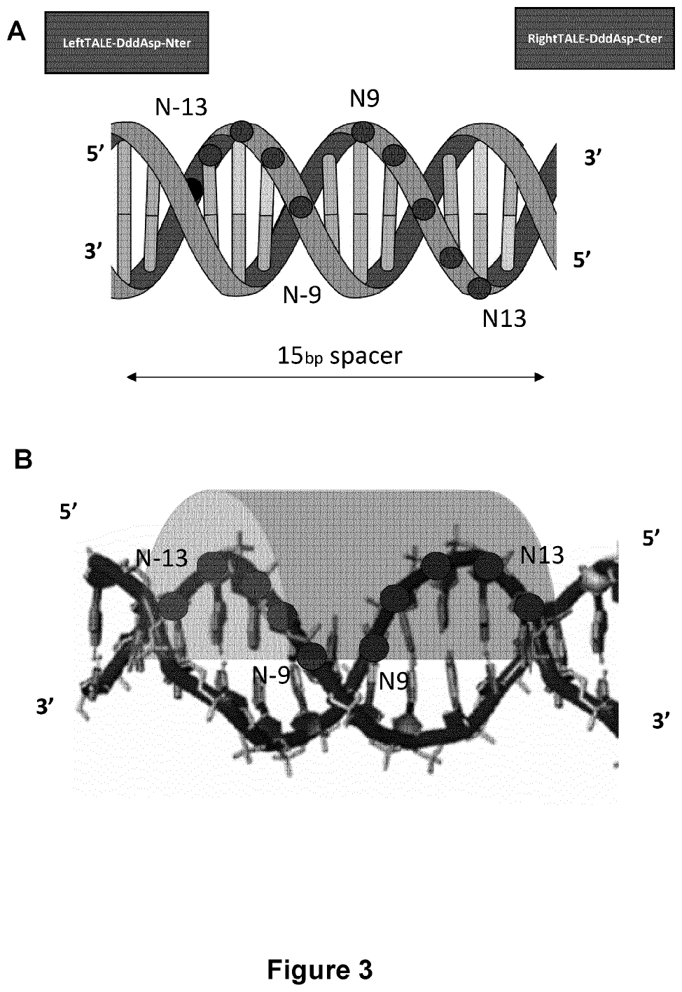

To define the key determinants for efficient TALEB editing (C-to-T conversion) using the previously described split-DddaTox strategy, we first selected a subset of 37 TALE-Nucleases that showed high activity (median=82% and s.d.=12) (FIG. 2A) in primary T-cells. These 37 target sequences (SEQ ID NO:33 to 69 in Table 1) were carefully chosen to target regions with different chromatin states in T cells. The spacer sequence, sequence between the two TALE binding regions, was also kept constant to 15 bp as it was previously shown to optimize TALE-Nuclease [Juillerat, A. et al. Comprehensive analysis of the specificity of transcription activator-like effector nucleases (2014) Nucleic Acids Research, 42(8):5390-5402). The sequence of the spacers contained various numbers, homogeneously distributed, of Cs, Gs, TCs or GAs as previous studies demonstrated a strong editing preference in 5′-TC-3′ contexts (Mok et al. A bacterial cytidine deaminase toxin enables CRISPR-free mitochondrial base editing (2020) Nature 583, 631-637). 37 TALE base editors with the DddAtox splits and an uracil glycosylase inhibitor (UGI), replacing the FokI catalytic domain, were produced as described in example 1. The G1397 split was used since this fusion showed better editing activity. The maximum editing within the spacer for a given TALE base editors was compared to the Indel frequencies created by the corresponding TALE-Nuclease counterpart (FIG. 2B). The complete lack of correlation (Spearman correlation=0.16, p-value=0.33) between the two data sets (TALE-Nuclease vs TALE base editing frequencies) suggests that the key determinant for efficient editing could be the positioning of the target cytosine within the spacer. Indeed, analysis of editing efficiency in function of the position within the spacer showed a defined 4-5 bp editing window on both, top and bottom strands (FIG. 3).

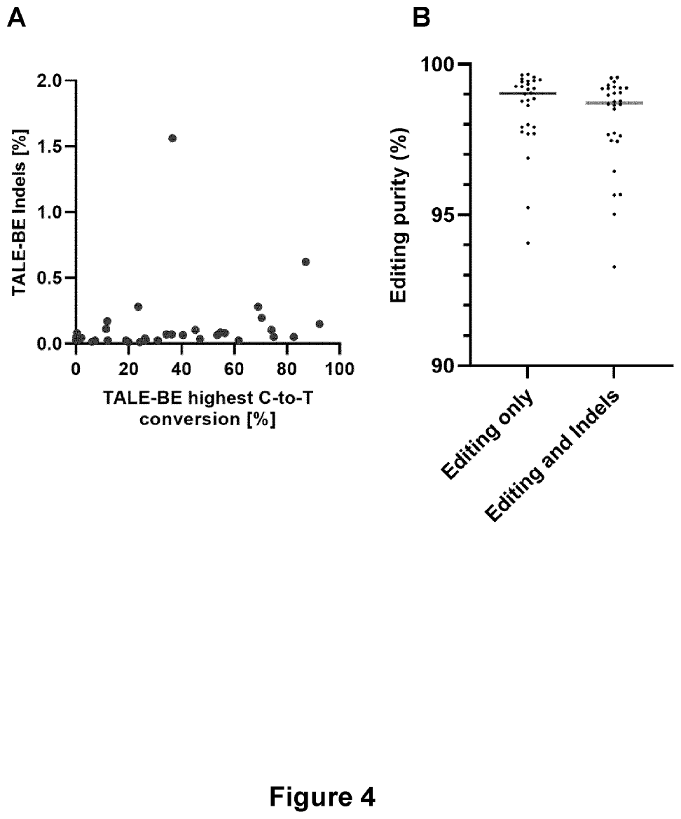

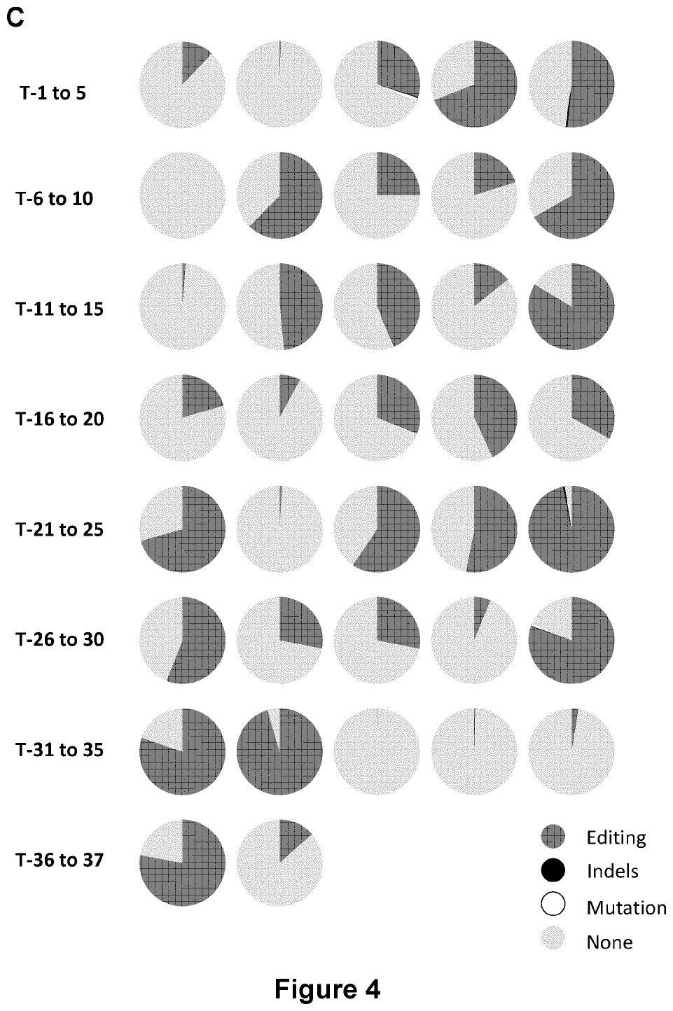

Interestingly, only low frequencies (<0.5%) of Indels (small insertion and deletions) were observed for 35 out of 37 base editors (Indel frequencies: median=0.06% and s.d.=0.17). The Indels at the target site moderately correlated with editing frequency within the spacers (Spearman correlation=0.44, p-value=0.007)) (FIG. 4A). In addition, we measured low byproduct (C-to-A/G) editing within the editing window, overall indicative of a very high final purity of the edited cell populations (FIG. 4B and FIG. 4C).

TABLE 1
Genomic Target sequences used in Example 2

| SEQ ID | | |
|---|---|---|
| NO :# | Name | Polynucleotide target sequences (LEFTspacerRIGHT) |
| 33 | T-1 | TGCCATCTGCTGGGTGCtgtcgtttgccatcgGCCTGACTCCCATGCTA |
| 34 | T-2 | TAGGTTGGAACAACTGCggtcagccaaaggagGGCAAGAACCACTCCCA |
| 35 | T-3 | TGGAGCCCTCGGCTCAAacctgggggcctggtACCCTGCGGCTCCCGAA |
| 36 | T-4 | TGAGCAGAGCAACCCTGccccccaggtccagaAACCGCGTGCCAAACCA |
| 37 | T-5 | TCCTCTGTGTCCCTGTGgtccctggagcagccGTTCCGCATCGAGCTCA |
| 38 | T-6 | TTTGCGGAATTGGAATTtcttagctgtgacacATCCAGGTTACATGGCA |
| 39 | T-7 | TTGGAATTTCTTAGCTGtgacacatccaggttACATGGCATTTCTCACA |
| 40 | T-8 | TGTGACACATCCAGGTTacatggcatttctcaCATATGATGAAGTTAAA |
| 41 | T-9 | TTATAGGCTTCTTCTCTggaatcttcttcatcATCCTCCTGACAATCGA |
| 42 | T-10 | TTTGCTGCCATTTCTGGaatgattctttcaatCATGGACATACTTAATA |
| 43 | T-11 | TGGACTGCCTGCCACTGccccggcgcatggccGACTACCTCCGACAGTA |
| 44 | T-12 | TGCGCCTAGTGACCCAGcactgcctgctcctcCACCAGCCACTGCTGTA |
| 45 | T-13 | TATTACCCAATGGGGACttggagaagcggagtGAGCCCCAGCCAGAGGA |
| 46 | T-14 | TGAATGCTGTGGAAGAAaaccaggggcccgggGAGTCTCAGAAGGTGGA |
| 47 | T-15 | TGATGGCCAATTCTGCCataagccctgtcctcCAGGTATGTTACACAAA |
| 48 | T-16 | TTAATGCCCAAGTGACTgacatcaactccaagGGATTGGAATTGAGGAA |
| 49 | T-17 | TGGGGATGAACCAGACTgcgtgccctgccaagAAGGGAAGGAGTACACA |
| 50 | T-18 | TGCAGAAGATGTAGATTgtgtgatgaaggacaTGGTAAGAGTCTTAAAA |
| 51 | T-19 | TTTCTAGATGTGAACATggaatcatcaaggaaTGCACACTCACCAGCAA |
| 52 | T-20 | TCTTGGGGGCCCCTTCCccacactatctcaatGCAAATATCTGTCTGAA |
| 53 | T-21 | TGCTACTGGCCAACACCacctccgccttccccTACGCGCTCCTGAGCAA |
| 54 | T-22 | TTTTAATAGCATTATTCaaccaagaagttcaaATTCCCTTGACCGGTAA |
| 55 | T-23 | TTCCAGAAAGTTACTGTggcccatgtcctaaaAACTGGATATGTTACAA |
| 56 | T-24 | TTAGGGGACCCATTAGGcatagaggactctctGGAAAGCCAAGATTCAA |
| 57 | T-25 | TCTAAGAAGTTCCTGCTctggagttgactaaaGAATGTGGTTAGAGACA |
| 58 | T-26 | TGCATATCTGGGCTCAGatgcttgtcattttcCAGTGATAACTCCATCA |
| 59 | T-27 | TGCTTGTCATTTTCCAGtgataactccatcaaTGCCTCCTAGTGGTATA |
| 60 | T-28 | TAGTGAACCTTCTCTCTctgggctccttcagaTCAAGAAATTGAAACAA |
| 61 | T-29 | TCCAGGTGAAAGCAGTCaaccaaatgtctccgATTTGAGTGATAAGAAA |
| 62 | T-30 | TGACGCCTGGCCGGCCGgccgcgggactatccACCTGCAAGACTATCGA |
| 63 | T-31 | TCGACATGGAGCTGGTGaagcggaagcgcatcGAGGCCATCCGCGGCCA |
| 64 | T-32 | TGAGGCCGACTACTACGccaaggaggtcacccGCGTGCTAATGGTGGAA |
| 65 | T-33 | TGATCGCCTCCCTTCATttctccctgctagaaATCTATGACAAGTTCAA |
| 66 | T-34 | TGGTTACCATTCTCTGTgtcaccccatgaaccATAATGGCCTGCTACCA |
| 67 | T-35 | TGCCACATGGCCAGCTGactaccattaaccagTCACAGCTAAGTGCTCA |
| 68 | T-36 | TCTGCCTATTCACCGATtttgattctcaaacaAATGTGTCACAAAGTAA |
| 69 | T-37 | TGAGGTCTATGGACTTCaagagcaacagtgctGTGGCCTGGAGCAACAA |

### Example 3: Screening and Rules for Optimal Base Editing

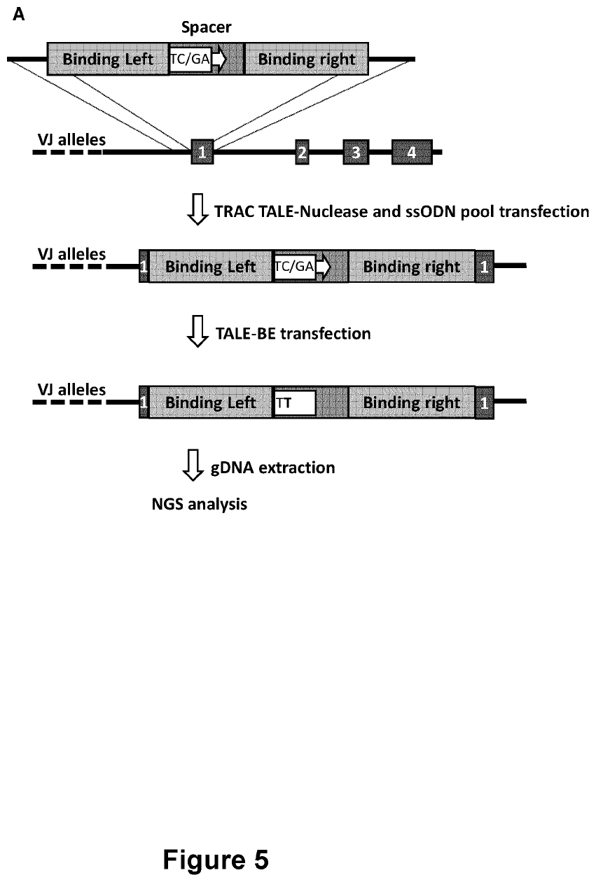

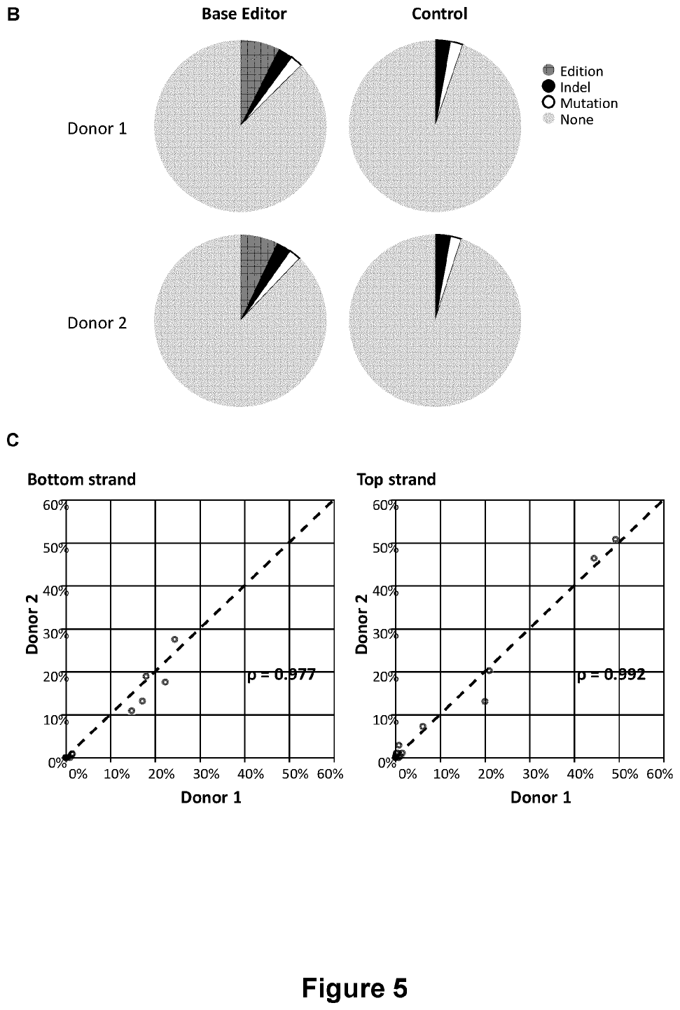

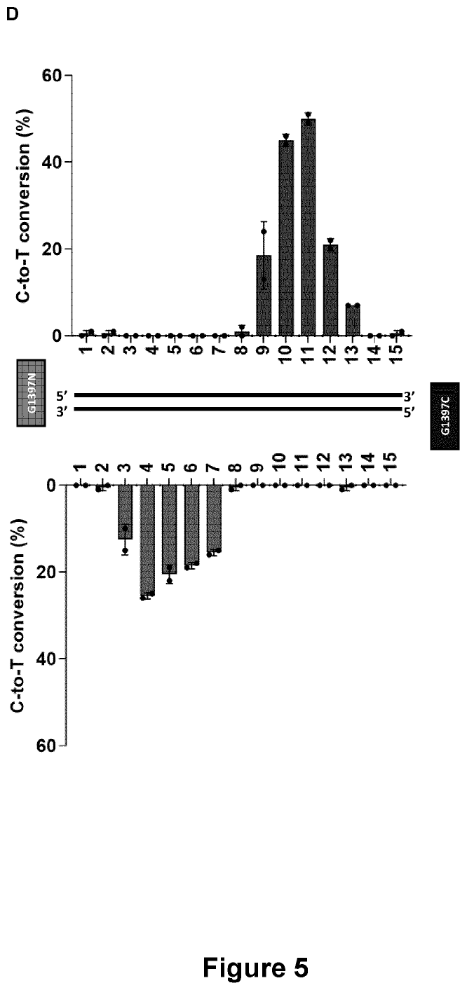

To more comprehensively investigate DddA-derived cytosine base editors, a medium to high throughput format screening, in a define genomic context, was designed by generating a pool of primary T-cells, containing predefined TALE base editors target sequences precisely inserted at the TRAC gene. Each of the TALE base editors targets containing a unique TO or GA (target for the DddA deaminase) within the spacer sequence flanked by two fixed TALE binding sequences (RVD-L and RVD-R, FIG. 5A). This setup allows the uniform TALE binding to the artificial target sites, excluding editing variability caused by (i) different DNA binding affinities from different TALE array protein and (ii) the impact of epigenomic factors, such as chromosome relaxation around the artificial base editors target sites.

A collection of 30 ssODNs was created comprising the previous polynucleotide TALE binding sequences of T-25 (SEQ ID NO:57 in Table 1) separated by 15 bp variable spacer sequences (similar to our previous collection of TALE base editors targeting endogenous loci) as represented below:

5′ TCTAAGAAGTTCCTGCT(variable spacer 15 nucleotides)GAATGTGGTTAGAGACA 3′
(SEQ ID NO: 70 to 100 in Table 2).

TABLE 2
Sequences of individual ssODN used to identifiy editing windows with a 15 bp spacer

| | SEQ ID | |
|---|---|---|
| | NO:# | Sequence |
| TCName-1 | 70 | GAGATCATGTCCTAACCCTGATCCTCTTGTCCCACAGATATCCAGAACCCTCTAA |
| | | GAAGTTCCTGCTCTAATATAAATATATGAATGTGGTTAGAGACATGACCCTGCCG |
| | | TGTACCAGCTGAGAGACTCTAAATCCAGTGACAAGTCTG |
| TC-2 | 71 | GAGATCATGTCCTAACCCTGATCCTCTTGTCCCACAGATATCCAGAACCCTCTAA |
| | | GAAGTTCCTGCTTCAATATAAATATATGAATGTGGTTAGAGACATGACCCTGCCG |
| | | TGTACCAGCTGAGAGACTCTAAATCCAGTGACAAGTCTG |
| TC-3 | 72 | GAGATCATGTCCTAACCCTGATCCTCTTGTCCCACAGATATCCAGAACCCTCTAA |
| | | GAAGTTCCTGCTATCATATAAATATATGAATGTGGTTAGAGACATGACCCTGCCG |
| | | TGTACCAGCTGAGAGACTCTAAATCCAGTGACAAGTCTG |
| TC-4 | 73 | GAGATCATGTCCTAACCCTGATCCTCTTGTCCCACAGATATCCAGAACCCTCTAA |
| | | GAAGTTCCTGCTAATCTATAAATATATGAATGTGGTTAGAGACATGACCCTGCCG |
| | | TGTACCAGCTGAGAGACTCTAAATCCAGTGACAAGTCTG |
| TC-5 | 74 | GAGATCATGTCCTAACCCTGATCCTCTTGTCCCACAGATATCCAGAACCCTCTA |
| | | AGAAGTTCCTGCTATATCATAAATATATGAATGTGGTTAGAGACATGACCCTGC |
| | | CGTGTACCAGCTGAGAGACTCTAAATCCAGTGACAAGTCTG |
| TC-6 | 75 | GAGATCATGTCCTAACCCTGATCCTCTTGTCCCACAGATATCCAGAACCCTCTA |
| | | AGAAGTTCCTGCTATAATCTAAATATATGAATGTGGTTAGAGACATGACCCTGC |
| | | CGTGTACCAGCTGAGAGACTCTAAATCCAGTGACAAGTCTG |
| TC-7 | 76 | GAGATCATGTCCTAACCCTGATCCTCTTGTCCCACAGATATCCAGAACCCTCTA |
| | | AGAAGTTCCTGCTAATAATCAAATATATGAATGTGGTTAGAGACATGACCCTGC |
| | | CGTGTACCAGCTGAGAGACTCTAAATCCAGTGACAAGTCTG |
| TC-8 | 78 | GAGATCATGTCCTAACCCTGATCCTCTTGTCCCACAGATATCCAGAACCCTCTA |
| | | AGAAGTTCCTGCTTATAAATCAATATATGAATGTGGTTAGAGACATGACCCTGC |
| | | CGTGTACCAGCTGAGAGACTCTAAATCCAGTGACAAGTCTG |
| TC-9 | 79 | GAGATCATGTCCTAACCCTGATCCTCTTGTCCCACAGATATCCAGAACCCTCTA |
| | | AGAAGTTCCTGCTTATAATATCATATATGAATGTGGTTAGAGACATGACCCTGC |
| | | CGTGTACCAGCTGAGAGACTCTAAATCCAGTGACAAGTCTG |
| TC-10 | 80 | GAGATCATGTCCTAACCCTGATCCTCTTGTCCCACAGATATCCAGAACCCTCTA |
| | | AGAAGTTCCTGCTTAATATAATCTATATGAATGTGGTTAGAGACATGACCCTGC |
| | | CGTGTACCAGCTGAGAGACTCTAAATCCAGTGACAAGTCTG |
| TC-11 | 81 | GAGATCATGTCCTAACCCTGATCCTCTTGTCCCACAGATATCCAGAACCCTCTA |
| | | AGAAGTTCCTGCTTAATATAAATCATATGAATGTGGTTAGAGACATGACCCTGC |
| | | CGTGTACCAGCTGAGAGACTCTAAATCCAGTGACAAGTCTG |
| TC-12 | 82 | GAGATCATGTCCTAACCCTGATCCTCTTGTCCCACAGATATCCAGAACCCTCTA |
| | | AGAAGTTCCTGCTAATAATAATATCTATGAATGTGGTTAGAGACATGACCCTGC |
| | | CGTGTACCAGCTGAGAGACTCTAAATCCAGTGACAAGTCTG |
| TC-13 | 83 | GAGATCATGTCCTAACCCTGATCCTCTTGTCCCACAGATATCCAGAACCCTCTA |
| | | AGAAGTTCCTGCTAATATAAATAATCATGAATGTGGTTAGAGACATGACCCTGC |
| | | CGTGTACCAGCTGAGAGACTCTAAATCCAGTGACAAGTCTG |
| TC-14 | 84 | GAGATCATGTCCTAACCCTGATCCTCTTGTCCCACAGATATCCAGAACCCTCTA |
| | | AGAAGTTCCTGCTAATATAAATATATCTGAATGTGGTTAGAGACATGACCCTGC |
| | | CGTGTACCAGCTGAGAGACTCTAAATCCAGTGACAAGTCTG |
| TC-15 | 85 | GAGATCATGTCCTAACCCTGATCCTCTTGTCCCACAGATATCCAGAACCCTCTA |
| | | AGAAGTTCCTGCTAATATAAATATAATCGAATGTGGTTAGAGACATGACCCTGC |
| | | CGTGTACCAGCTGAGAGACTCTAAATCCAGTGACAAGTCTG |
| GA-1 | 86 | GAGATCATGTCCTAACCCTGATCCTCTTGTCCCACAGATATCCAGAACCCTCTA |
| | | AGAAGTTCCTGCTATATATTTATATTAGGAATGTGGTTAGAGACATGACCCTGC |
| | | CGTGTACCAGCTGAGAGACTCTAAATCCAGTGACAAGTCTG |
| GA-2 | 87 | GAGATCATGTCCTAACCCTGATCCTCTTGTCCCACAGATATCCAGAACCCTCTA |
| | | AGAAGTTCCTGCTATATATTTATATTGAGAATGTGGTTAGAGACATGACCCTGC |
| | | CGTGTACCAGCTGAGAGACTCTAAATCCAGTGACAAGTCTG |
| GA-3 | 88 | GAGATCATGTCCTAACCCTGATCCTCTTGTCCCACAGATATCCAGAACCCTCTA |
| | | AGAAGTTCCTGCTATATATTTATATGATGAATGTGGTTAGAGACATGACCCTGC |
| | | CGTGTACCAGCTGAGAGACTCTAAATCCAGTGACAAGTCTG |
| GA-4 | 89 | GAGATCATGTCCTAACCCTGATCCTCTTGTCCCACAGATATCCAGAACCCTCTA |
| | | AGAAGTTCCTGCTATATATTTATAGATTGAATGTGGTTAGAGACATGACCCTGC |
| | | CGTGTACCAGCTGAGAGACTCTAAATCCAGTGACAAGTCTG |
| GA-5 | 90 | GAGATCATGTCCTAACCCTGATCCTCTTGTCCCACAGATATCCAGAACCCTCTA |
| | | AGAAGTTCCTGCTATATATTTATGATATGAATGTGGTTAGAGACATGACCCTGC |
| | | CGTGTACCAGCTGAGAGACTCTAAATCCAGTGACAAGTCTG |
| GA-6 | 91 | GAGATCATGTCCTAACCCTGATCCTCTTGTCCCACAGATATCCAGAACCCTCTA |
| | | AGAAGTTCCTGCTATATATTTAGATTATGAATGTGGTTAGAGACATGACCCTGC |
| | | CGTGTACCAGCTGAGAGACTCTAAATCCAGTGACAAGTCTG |
| GA-7 | 92 | GAGATCATGTCCTAACCCTGATCCTCTTGTCCCACAGATATCCAGAACCCTCTA |
| | | AGAAGTTCCTGCTATATATTTGATTATTGAATGTGGTTAGAGACATGACCCTGC |
| | | CGTGTACCAGCTGAGAGACTCTAAATCCAGTGACAAGTCTG |
| GA-8 | 93 | GAGATCATGTCCTAACCCTGATCCTCTTGTCCCACAGATATCCAGAACCCTCTA |
| | | AGAAGTTCCTGCTATATATTGATTTATAGAATGTGGTTAGAGACATGACCCTGC |
| | | CGTGTACCAGCTGAGAGACTCTAAATCCAGTGACAAGTCTG |
| GA-9 | 94 | GAGATCATGTCCTAACCCTGATCCTCTTGTCCCACAGATATCCAGAACCCTCTA |
| | | AGAAGTTCCTGCTATATATGATATTATAGAATGTGGTTAGAGACATGACCCTGC |
| | | CGTGTACCAGCTGAGAGACTCTAAATCCAGTGACAAGTCTG |
| GA-10 | 95 | GAGATCATGTCCTAACCCTGATCCTCTTGTCCCACAGATATCCAGAACCCTCTA |
| | | AGAAGTTCCTGCTATATAGATTATATTAGAATGTGGTTAGAGACATGACCCTGC |
| | | CGTGTACCAGCTGAGAGACTCTAAATCCAGTGACAAGTCTG |
| GA-11 | 96 | GAGATCATGTCCTAACCCTGATCCTCTTGTCCCACAGATATCCAGAACCCTCTA |
| | | AGAAGTTCCTGCTATATGATTTATATTAGAATGTGGTTAGAGACATGACCCTGC |
| | | CGTGTACCAGCTGAGAGACTCTAAATCCAGTGACAAGTCTG |
| GA-12 | 97 | GAGATCATGTCCTAACCCTGATCCTCTTGTCCCACAGATATCCAGAACCCTCTA |
| | | AGAAGTTCCTGCTATAGATATTATTATTGAATGTGGTTAGAGACATGACCCTGC |
| | | CGTGTACCAGCTGAGAGACTCTAAATCCAGTGACAAGTCTG |
| GA-13 | 98 | GAGATCATGTCCTAACCCTGATCCTCTTGTCCCACAGATATCCAGAACCCTCTA |
| | | AGAAGTTCCTGCTATGATTATTTATATTGAATGTGGTTAGAGACATGACCCTGC |
| | | CGTGTACCAGCTGAGAGACTCTAAATCCAGTGACAAGTCTG |
| GA-14 | 99 | GAGATCATGTCCTAACCCTGATCCTCTTGTCCCACAGATATCCAGAACCCTCTA |
| | | AGAAGTTCCTGCTAGATATATTTATATTGAATGTGGTTAGAGACATGACCCTGC |
| | | CGTGTACCAGCTGAGAGACTCTAAATCCAGTGACAAGTCTG |
| GA-15 | 100 | GAGATCATGTCCTAACCCTGATCCTCTTGTCCCACAGATATCCAGAACCCTCTA |
| | | AGAAGTTCCTGCTGATTATATTTATATTGAATGTGGTTAGAGACATGACCCTGC |
| | | CGTGTACCAGCTGAGAGACTCTAAATCCAGTGACAAGTCTG |

These SSODNs were used to generate a pool of primary T-cells harboring a collection of base editor targets. The 30 ssODN oligonucleotides were mixed in equal amount and transfected in primary T-cells by electroporation (200 pmol per million cells) simultaneously with mRNA encoding the TALE-Nucleases targeting TRAC (Left TALEN monomer of SEQ ID NO:16 and right TALEN monomer of SEQ ID NO:17). In a second step, two days post transfection of the ssODN pool, the mRNAs encoding the T-25 TALE base editors were vectorized by electroporation. The genomic DNA of transfected cells was then harvested at day 2 post TALE base editors transfection for editing analysis (FIG. 5A). The NGS analysis showed that the ssODNs were efficiently and homogenously integrated at the TRAC locus (read number: median=1667.5, mean=1686.2, s.d.=351.7). The control sample treated without TALE base editors showed low frequencies of background mutations, whereas the samples treated with TALE base editors showed detectable and reproducible levels of C-to-T conversion (FIG. 5B and FIG. 5C). The analysis further highlighted editing windows comparable to those observed with the 37 TALE base editors targeting endogenous sequences (FIG. 5D), altogether validating this pooled approach.

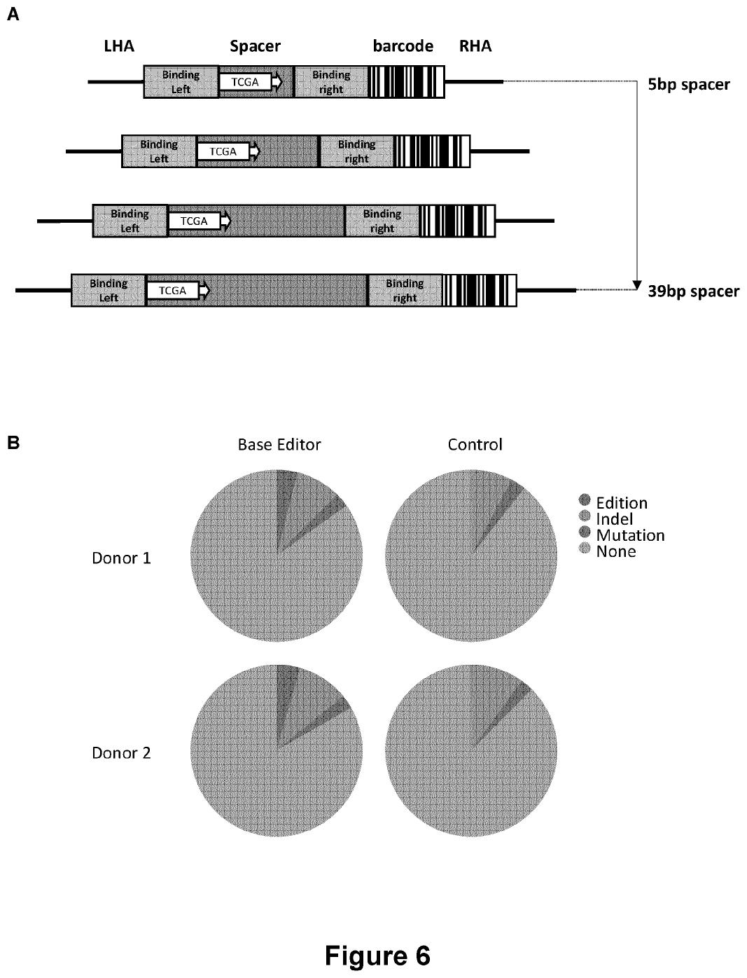

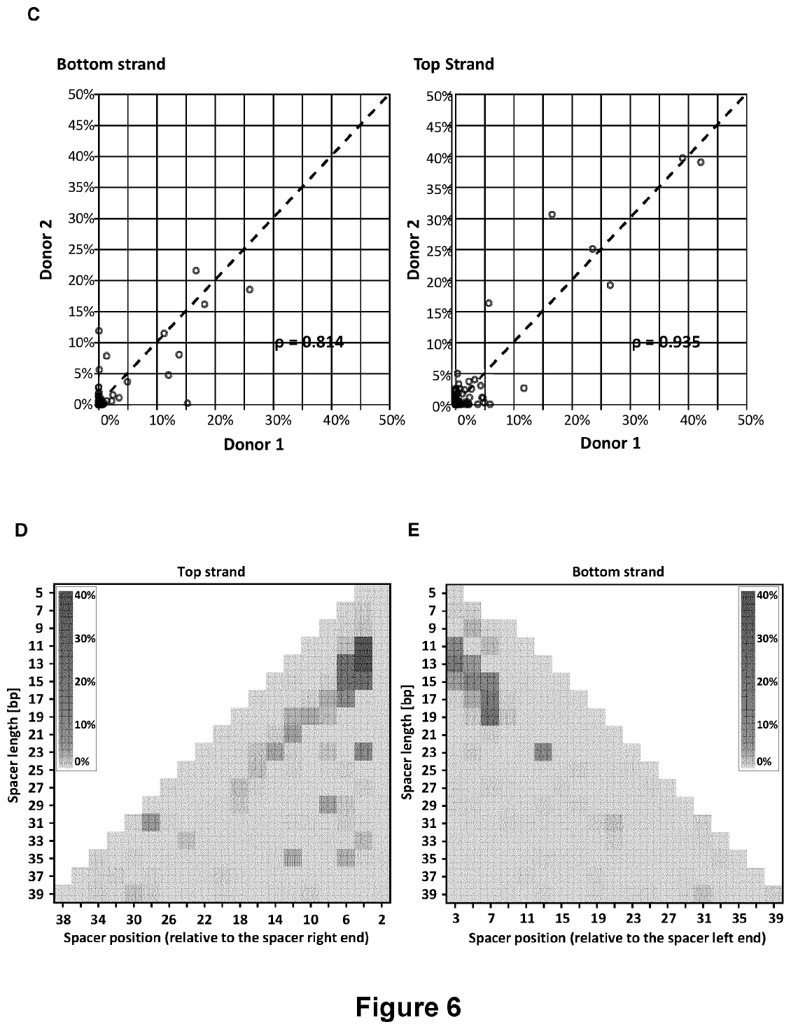

The ssODN collection was expanded to spacers with various number length, spanning from 5 to 39 bp (i.e. 5, 7, 9, 11 . . . 37, 39 bp). A TCGA quadruplex target sequence was incorporated in the spacer at every other position (FIG. 6A). This design, containing 191 unique ssODNs (SEQ ID NO: 103 to 293, in Table 3), allowed to interrogate simultaneously editing efficiencies on both strands with a single ssODN. Additionally, to facilitate the sequence analysis, a unique barcode was added to each construct (FIG. 6A). Upon filtering the NGS data to remove the reads in which the barcode conflicted with the spacer sequence, a high and homogenous representation of each ssODN was obtained (read number: median=545, mean=3522.6, s.d.=7122.5). As with the previous collection (15 bp spacer), low frequencies of mutations were observed without the TALE base editors while C-to-T conversion was robustly measured with the TALE base editors, either on the plus or minus strand (FIGS. 6B and 6C). Analysis of the data pointed out a spacer length ranging from 11 to 17 bp to achieve optimal editing, with a 4-5 bp editing windows on the different spacers (FIG. 6D and FIG. 6E).

To Investigate the impact of the sequences surrounding the TC context on base editing efficiency, a further collection of ssODNs that contains two fixed TALE array protein binding sites from the T-25 TALE base editors (SEQ ID NO:57 of Table 1) separated by a 16 bp spacer sequences was designed (SEQ ID NO:294 to 357 in Table 4) The spacer sequences were composed of a 10 bp molecular barcode followed by an NTCCNN sequence (target of the based editors). Cell handling, transfection and gDNA analysis was performed as previously described.

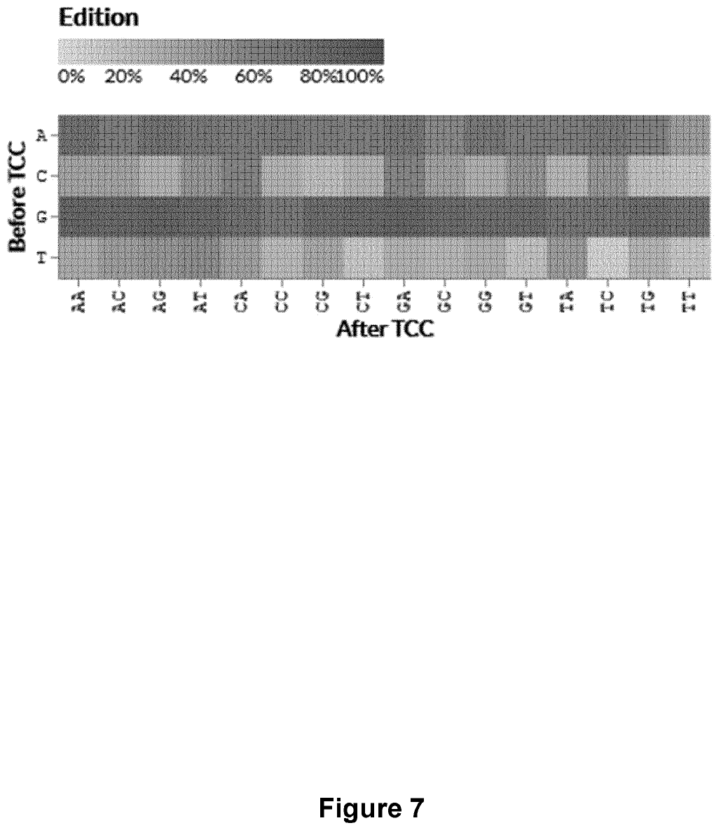

After filtering the NGS data and analysis, the results clearly demonstrated that a G or an A before the TC favored efficient editing (FIG. 7).

TABLE 3
Sequences of individual ssODN used to asses effect of spacer length on editing

| | SEQ | |
|---|---|---|
| | ID | |
| Name | NO:# | Sequence |
| TCGA-1 | 103 | GAGATCATGTCCTAACCCTGATCCTCTTGTCCCACAGATATCCAGAACCCTCTAAGAAGTTCCTGCTTCGATGAATGTGGTTAGAGACAAAACAGTGACCCTGCCGTGTACCAGCTGAGAGACTCT |
| | | AAATCCAGTGACAAGTCTG |
| TCGA-2 | 104 | GAGATCATGTCCTAACCCTGATCCTCTTGTCCCACAGATATCCAGAACCCTCTAAGAAGTTCCTGCTATCGAGAATGTGGTTAGAGACAAAACCTTGACCCTGCCGTGTACCAGCTGAGAGACTCT |
| | | AAATCCAGTGACAAGTCTG |
| TCGA-3 | 105 | GAGATCATGTCCTAACCCTGATCCTCTTGTCCCACAGATATCCAGAACCCTCTAAGAAGTTCCTGCTGATCAGAATGTGGTTAGAGACAAAACGATGACCCTGCCGTGTACCAGCTGAGAGACTCT |
| | | AAATCCAGTGACAAGTCTG |
| TCGA-4 | 106 | GAGATCATGTCCTAACCCTGATCCTCTTGTCCCACAGATATCCAGAACCCTCTAAGAAGTTCCTGCTAGATCGAATGTGGTTAGAGACAAAACGGTGACCCTGCCGTGTACCAGCTGAGAGACTCT |
| | | AAATCCAGTGACAAGTCTG |
| TCGA-5 | 107 | GAGATCATGTCCTAACCCTGATCCTCTTGTCCCACAGATATCCAGAACCCTCTAAGAAGTTCCTGCTTCGATATGAATGTGGTTAGAGACAAAACTCTGACCCTGCCGTGTACCAGCTGAGAGACT |
| | | CTAAATCCAGTGACAAGTCTG |
| TCGA-6 | 108 | GAGATCATGTCCTAACCCTGATCCTCTTGTCCCACAGATATCCAGAACCCTCTAAGAAGTTCCTGCTAATCGATGAATGTGGTTAGAGACAAAAGACTGACCCTGCCGTGTACCAGCTGAGAGACT |
| | | CTAAATCCAGTGACAAGTCTG |
| TCGA-7 | 109 | GAGATCATGTCCTAACCCTGATCCTCTTGTCCCACAGATATCCAGAACCCTCTAAGAAGTTCCTGCTGATATCAGAATGTGGTTAGAGACAAAAGAGTGACCCTGCCGTGTACCAGCTGAGAGAC |
| | | TCTAAATCCAGTGACAAGTCTG |
| TCGA-8 | 110 | GAGATCATGTCCTAACCCTGATCCTCTTGTCCCACAGATATCCAGAACCCTCTAAGAAGTTCCTGCTTCGAtattaGAATGTGGTTAGAGACAAAAGCTTGACCCTGCCGTGTACCAGCTGAGAGAC |
| | | TCTAAATCCAGTGACAAGTCTG |
| TCGA-9 | 111 | GAGATCATGTCCTAACCCTGATCCTCTTGTCCCACAGATATCCAGAACCCTCTAAGAAGTTCCTGCTtaTCGAtatGAATGTGGTTAGAGACAAAAGGATGACCCTGCCGTGTACCAGCTGAGAGAC |
| | | TCTAAATCCAGTGACAAGTCTG |
| TCGA-10 | 112 | GAGATCATGTCCTAACCCTGATCCTCTTGTCCCACAGATATCCAGAACCCTCTAAGAAGTTCCTGCTattaTCGAtGAATGTGGTTAGAGACAAAAGGGTGACCCTGCCGTGTACCAGCTGAGAGAC |
| | | TCTAAATCCAGTGACAAGTCTG |
| TCGA-11 | 113 | GAGATCATGTCCTAACCCTGATCCTCTTGTCCCACAGATATCCAGAACCCTCTAAGAAGTTCCTGCTAtattaTCGGAATGTGGTTAGAGACAAAAGTCTGACCCTGCCGTGTACCAGCTGAGAGAC |
| | | TCTAAATCCAGTGACAAGTCTG |
| TCGA-12 | 114 | GAGATCATGTCCTAACCCTGATCCTCTTGTCCCACAGATATCCAGAACCCTCTAAGAAGTTCCTGCTTCGAtatttaaGAATGTGGTTAGAGACAAAATCCTGACCCTGCCGTGTACCAGCTGAGAG |
| | | ACTCTAAATCCAGTGACAAGTCTG |
| TCGA-13 | 115 | GAGATCATGTCCTAACCCTGATCCTCTTGTCCCACAGATATCCAGAACCCTCTAAGAAGTTCCTGCTaaTCGAtatttGAATGTGGTTAGAGACAAAATGCTGACCCTGCCGTGTACCAGCTGAGAG |
| | | ACTCTAAATCCAGTGACAAGTCTG |
| TCGA-14 | 116 | GAGATCATGTCCTAACCCTGATCCTCTTGTCCCACAGATATCCAGAACCCTCTAAGAAGTTCCTGCTttaaTCGAtatGAATGTGGTTAGAGACAAACAAGTGACCCTGCCGTGTACCAGCTGAGAG |
| | | ACTCTAAATCCAGTGACAAGTCTG |
| TCGA-15 | 117 | GAGATCATGTCCTAACCCTGATCCTCTTGTCCCACAGATATCCAGAACCCTCTAAGAAGTTCCTGCTatttaaTCGAtGAATGTGGTTAGAGACAAACACCTGACCCTGCCGTGTACCAGCTGAGAG |
| | | ACTCTAAATCCAGTGACAAGTCTG |
| TCGA-16 | 118 | GAGATCATGTCCTAACCCTGATCCTCTTGTCCCACAGATATCCAGAACCCTCTAAGAAGTTCCTGCTAtatttaaTCGGAATGTGGTTAGAGACAAACAGATGACCCTGCCGTGTACCAGCTGAGAG |
| | | ACTCTAAATCCAGTGACAAGTCTG |
| TCGA-17 | 119 | GAGATCATGTCCTAACCCTGATCCTCTTGTCCCACAGATATCCAGAACCCTCTAAGAAGTTCCTGCTTCGAtaattataaGAATGTGGTTAGAGACAAACAGGTGACCCTGCCGTGTACCAGCTGAG |
| | | AGACTCTAAATCCAGTGACAAGTCTG |
| TCGA-18 | 120 | GAGATCATGTCCTAACCCTGATCCTCTTGTCCCACAGATATCCAGAACCCTCTAAGAAGTTCCTGCTaaTCGAtaattatGAATGTGGTTAGAGACAAACCATTGACCCTGCCGTGTACCAGCTGAG |
| | | AGACTCTAAATCCAGTGACAAGTCTG |
| TCGA-19 | 121 | GAGATCATGTCCTAACCCTGATCCTCTTGTCCCACAGATATCCAGAACCCTCTAAGAAGTTCCTGCTataaTCGAtaattGAATGTGGTTAGAGACAAACCGTTGACCCTGCCGTGTACCAGCTGAG |
| | | AGACTCTAAATCCAGTGACAAGTCTG |
| TCGA-20 | 122 | GAGATCATGTCCTAACCCTGATCCTCTTGTCCCACAGATATCCAGAACCCTCTAAGAAGTTCCTGCTttataaTCGAtaaGAATGTGGTTAGAGACAAACGCATGACCCTGCCGTGTACCAGCTGAG |
| | | AGACTCTAAATCCAGTGACAAGTCTG |
| TCGA-21 | 123 | GAGATCATGTCCTAACCCTGATCCTCTTGTCCCACAGATATCCAGAACCCTCTAAGAAGTTCCTGCTaattataaTCGAtGAATGTGGTTAGAGACAAACTCGTGACCCTGCCGTGTACCAGCTGAG |
| | | AGACTCTAAATCCAGTGACAAGTCTG |
| TCGA-22 | 124 | GAGATCATGTCCTAACCCTGATCCTCTTGTCCCACAGATATCCAGAACCCTCTAAGAAGTTCCTGCTAtaattataaTCGGAATGTGGTTAGAGACAAACTGGTGACCCTGCCGTGTACCAGCTGAG |
| | | AGACTCTAAATCCAGTGACAAGTCTG |
| TCGA-23 | 125 | GAGATCATGTCCTAACCCTGATCCTCTTGTCCCACAGATATCCAGAACCCTCTAAGAAGTTCCTGCTTCGAttataattaaaGAATGTGGTTAGAGACAAACTTCTGACCCTGCCGTGTACCAGCTG |
| | | AGAGACTCTAAATCCAGTGACAAGTCTG |
| TCGA-24 | 126 | GAGATCATGTCCTAACCCTGATCCTCTTGTCCCACAGATATCCAGAACCCTCTAAGAAGTTCCTGCTaaTCGAttataattaGAATGTGGTTAGAGACAAAGAACTGACCCTGCCGTGTACCAGCTG |
| | | AGAGACTCTAAATCCAGTGACAAGTCTG |
| TCGA-25 | 127 | GAGATCATGTCCTAACCCTGATCCTCTTGTCCCACAGATATCCAGAACCCTCTAAGAAGTTCCTGCTtaaaTCGAttataatGAATGTGGTTAGAGACAAAGAAGTGACCCTGCCGTGTACCAGCTG |
| | | AGAGACTCTAAATCCAGTGACAAGTCTG |
| TCGA-26 | 128 | GAGATCATGTCCTAACCCTGATCCTCTTGTCCCACAGATATCCAGAACCCTCTAAGAAGTTCCTGCTattaaaTCGAttataGAATGTGGTTAGAGACAAAGACATGACCCTGCCGTGTACCAGCTG |
| | | AGAGACTCTAAATCCAGTGACAAGTCTG |
| TCGA-27 | 129 | GAGATCATGTCCTAACCCTGATCCTCTTGTCCCACAGATATCCAGAACCCTCTAAGAAGTTCCTGCTtaattaaaTCGAttaGAATGTGGTTAGAGACAAAGACTTGACCCTGCCGTGTACCAGCTG |
| | | AGAGACTCTAAATCCAGTGACAAGTCTG |
| TCGA-28 | 130 | GAGATCATGTCCTAACCCTGATCCTCTTGTCCCACAGATATCCAGAACCCTCTAAGAAGTTCCTGCTtataattaaaTCGAtGAATGTGGTTAGAGACAAAGAGATGACCCTGCCGTGTACCAGCTGA |
| | | GAGACTCTAAATCCAGTGACAAGTCTG |
| TCGA-29 | 131 | GAGATCATGTCCTAACCCTGATCCTCTTGTCCCACAGATATCCAGAACCCTCTAAGAAGTTCCTGCTAttataattaaaTCGGAATGTGGTTAGAGACAAAGAGTTGACCCTGCCGTGTACCAGCTGA |
| | | GAGACTCTAAATCCAGTGACAAGTCTG |
| TCGA-30 | 132 | GAGATCATGTCCTAACCCTGATCCTCTTGTCCCACAGATATCCAGAACCCTCTAAGAAGTTCCTGCTTCGAtattattaaattaGAATGTGGTTAGAGACAAAGATCTGACCCTGCCGTGTACCAGCTG |
| | | AGAGACTCTAAATCCAGTGACAAGTCTG |
| TCGA-31 | 133 | GAGATCATGTCCTAACCCTGATCCTCTTGTCCCACAGATATCCAGAACCCTCTAAGAAGTTCCTGCTtaTCGAtattattaaatGAATGTGGTTAGAGACAAAGCTGTGACCCTGCCGTGTACCAGCTG |
| | | AGAGACTCTAAATCCAGTGACAAGTCTG |
| TCGA-32 | 134 | GAGATCATGTCCTAACCCTGATCCTCTTGTCCCACAGATATCCAGAACCCTCTAAGAAGTTCCTGCTattaTCGAtattattaaGAATGTGGTTAGAGACAAAGGAATGACCCTGCCGTGTACCAGCTG |
| | | AGAGACTCTAAATCCAGTGACAAGTCTG |
| TCGA-33 | 135 | GAGATCATGTCCTAACCCTGATCCTCTTGTCCCACAGATATCCAGAACCCTCTAAGAAGTTCCTGCTaaattaTCGAtattattGAATGTGGTTAGAGACAAAGGAGTGACCCTGCCGTGTACCAGCTG |
| | | AGAGACTCTAAATCCAGTGACAAGTCTG |
| TCGA-34 | 136 | GAGATCATGTCCTAACCCTGATCCTCTTGTCCCACAGATATCCAGAACCCTCTAAGAAGTTCCTGCTttaaattaTCGAtattaGAATGTGGTTAGAGACAAAGGGATGACCCTGCCGTGTACCAGCTG |
| | | AGAGACTCTAAATCCAGTGACAAGTCTG |
| TCGA-35 | 137 | GAGATCATGTCCTAACCCTGATCCTCTTGTCCCACAGATATCCAGAACCCTCTAAGAAGTTCCTGCTtattaaattaTCGAtatGAATGTGGTTAGAGACAAAGGGTTGACCCTGCCGTGTACCAGCTG |
| | | AGAGACTCTAAATCCAGTGACAAGTCTG |
| TCGA-36 | 138 | GAGATCATGTCCTAACCCTGATCCTCTTGTCCCACAGATATCCAGAACCCTCTAAGAAGTTCCTGCTattattaaattaTCGAtGAATGTGGTTAGAGACAAAGGTCTGACCCTGCCGTGTACCAGCTG |
| | | AGAGACTCTAAATCCAGTGACAAGTCTG |
| TCGA-37 | 139 | GAGATCATGTCCTAACCCTGATCCTCTTGTCCCACAGATATCCAGAACCCTCTAAGAAGTTCCTGCTAtattattaaattaTCGGAATGTGGTTAGAGACAAAGGTGTGACCCTGCCGTGTACCAGCTG |
| | | AGAGACTCTAAATCCAGTGACAAGTCTG |
| TCGA-38 | 140 | GAGATCATGTCCTAACCCTGATCCTCTTGTCCCACAGATATCCAGAACCCTCTAAGAAGTTCCTGCTTCGAtataattatattataGAATGTGGTTAGAGACAAAGTACTGACCCTGCCGTGTACCAGC |
| | | TGAGAGACTCTAAATCCAGTGACAAGTCTG |
| TCGA-39 | 141 | GAGATCATGTCCTAACCCTGATCCTCTTGTCCCACAGATATCCAGAACCCTCTAAGAAGTTCCTGCTtaTCGAtataattatattaGAATGTGGTTAGAGACAAAGTCGTGACCCTGCCGTGTACCAGC |
| | | TGAGAGACTCTAAATCCAGTGACAAGTCTG |
| TCGA-40 | 142 | GAGATCATGTCCTAACCCTGATCCTCTTGTCCCACAGATATCCAGAACCCTCTAAGAAGTTCCTGCTtataTCGAtataattatatGAATGTGGTTAGAGACAAATACCTGACCCTGCCGTGTACCAGC |
| | | TGAGAGACTCTAAATCCAGTGACAAGTCTG |
| TCGA-41 | 143 | GAGATCATGTCCTAACCCTGATCCTCTTGTCCCACAGATATCCAGAACCCTCTAAGAAGTTCCTGCTattataTCGAtataattatGAATGTGGTTAGAGACAAATCGATGACCCTGCCGTGTACCAGC |
| | | TGAGAGACTCTAAATCCAGTGACAAGTCTG |
| TCGA-42 | 144 | GAGATCATGTCCTAACCCTGATCCTCTTGTCCCACAGATATCCAGAACCCTCTAAGAAGTTCCTGCTatattataTCGAtataattGAATGTGGTTAGAGACAAATCTCTGACCCTGCCGTGTACCAGC |
| | | TGAGAGACTCTAAATCCAGTGACAAGTCTG |
| TCGA-43 | 145 | GAGATCATGTCCTAACCCTGATCCTCTTGTCCCACAGATATCCAGAACCCTCTAAGAAGTTCCTGCTttatattataTCGAtataaGAATGTGGTTAGAGACAAATTCCTGACCCTGCCGTGTACCAGC |
| | | TGAGAGACTCTAAATCCAGTGACAAGTCTG |
| TCGA-44 | 146 | GAGATCATGTCCTAACCCTGATCCTCTTGTCCCACAGATATCCAGAACCCTCTAAGAAGTTCCTGCTaattatattataTCGAtatGAATGTGGTTAGAGACAAATTGCTGACCCTGCCGTGTACCAGC |
| | | TGAGAGACTCTAAATCCAGTGACAAGTCTG |
| TCGA-45 | 147 | GAGATCATGTCCTAACCCTGATCCTCTTGTCCCACAGATATCCAGAACCCTCTAAGAAGTTCCTGCTataattatattataTCGAtGAATGTGGTTAGAGACAAATTGGTGACCCTGCCGTGTACCAGC |
| | | TGAGAGACTCTAAATCCAGTGACAAGTCTG |
| TCGA-46 | 148 | GAGATCATGTCCTAACCCTGATCCTCTTGTCCCACAGATATCCAGAACCCTCTAAGAAGTTCCTGCTAtataattatattataTCGGAATGTGGTTAGAGACAACAACGTGACCCTGCCGTGTACCAGC |
| | | TGAGAGACTCTAAATCCAGTGACAAGTCTG |
| TCGA-47 | 149 | GAGATCATGTCCTAACCCTGATCCTCTTGTCCCACAGATATCCAGAACCCTCTAAGAAGTTCCTGCTTCGAtataaatatattatttaGAATGTGGTTAGAGACAACAAGCTGACCCTGCCGTGTACCA |
| | | GCTGAGAGACTCTAAATCCAGTGACAAGTCTG |
| TCGA-48 | 150 | GAGATCATGTCCTAACCCTGATCCTCTTGTCCCACAGATATCCAGAACCCTCTAAGAAGTTCCTGCTtaTCGAtataaatatattattGAATGTGGTTAGAGACAACAAGTTGACCCTGCCGTGTACCA |
| | | GCTGAGAGACTCTAAATCCAGTGACAAGTCTG |
| TCGA-49 | 151 | GAGATCATGTCCTAACCCTGATCCTCTTGTCCCACAGATATCCAGAACCCTCTAAGAAGTTCCTGCTtttaTCGAtataaatatattaGAATGTGGTTAGAGACAACACCATGACCCTGCCGTGTACCA |
| | | GCTGAGAGACTCTAAATCCAGTGACAAGTCTG |
| TCGA-50 | 152 | GAGATCATGTCCTAACCCTGATCCTCTTGTCCCACAGATATCCAGAACCCTCTAAGAAGTTCCTGCTtatttaTCGAtataaatatatGAATGTGGTTAGAGACAACACTGTGACCCTGCCGTGTACCA |
| | | GCTGAGAGACTCTAAATCCAGTGACAAGTCTG |
| TCGA-51 | 153 | GAGATCATGTCCTAACCCTGATCCTCTTGTCCCACAGATATCCAGAACCCTCTAAGAAGTTCCTGCTattatttaTCGAtataaatatGAATGTGGTTAGAGACAACAGTGTGACCCTGCCGTGTACCA |
| | | GCTGAGAGACTCTAAATCCAGTGACAAGTCTG |
| TCGA-52 | 154 | GAGATCATGTCCTAACCCTGATCCTCTTGTCCCACAGATATCCAGAACCCTCTAAGAAGTTCCTGCTatattatttaTCGAtataaatGAATGTGGTTAGAGACAACATAGTGACCCTGCCGTGTACCA |
| | | GCTGAGAGACTCTAAATCCAGTGACAAGTCTG |
| TCGA-53 | 155 | GAGATCATGTCCTAACCCTGATCCTCTTGTCCCACAGATATCCAGAACCCTCTAAGAAGTTCCTGCTatatattatttaTCGAtataaGAATGTGGTTAGAGACAACATCTTGACCCTGCCGTGTACCA |
| | | GCTGAGAGACTCTAAATCCAGTGACAAGTCTG |
| TCGA-54 | 156 | GAGATCATGTCCTAACCCTGATCCTCTTGTCCCACAGATATCCAGAACCCTCTAAGAAGTTCCTGCTaaatatattatttaTCGAtatGAATGTGGTTAGAGACAACCGAATGACCCTGCCGTGTACCA |
| | | GCTGAGAGACTCTAAATCCAGTGACAAGTCTG |
| TCGA-55 | 157 | GAGATCATGTCCTAACCCTGATCCTCTTGTCCCACAGATATCCAGAACCCTCTAAGAAGTTCCTGCTataaatatattatttaTCGAtGAATGTGGTTAGAGACAACCTACTGACCCTGCCGTGTACCA |
| | | GCTGAGAGACTCTAAATCCAGTGACAAGTCTG |
| TCGA-56 | 158 | GAGATCATGTCCTAACCCTGATCCTCTTGTCCCACAGATATCCAGAACCCTCTAAGAAGTTCCTGCTAtataaatatattatttaTCGGAATGTGGTTAGAGACAACCTGATGACCCTGCCGTGTACCA |
| | | GCTGAGAGACTCTAAATCCAGTGACAAGTCTG |
| TCGA-57 | 159 | GAGATCATGTCCTAACCCTGATCCTCTTGTCCCACAGATATCCAGAACCCTCTAAGAAGTTCCTGCTTCGAtattaaatatattaatttaGAATGTGGTTAGAGACAACCTGTTGACCCTGCCGTGTAC |
| | | CAGCTGAGAGACTCTAAATCCAGTGACAAGTCTG |
| TCGA-58 | 160 | GAGATCATGTCCTAACCCTGATCCTCTTGTCCCACAGATATCCAGAACCCTCTAAGAAGTTCCTGCTtaTCGAtattaaatatattaattGAATGTGGTTAGAGACAACGAATTGACCCTGCCGTGTAC |
| | | CAGCTGAGAGACTCTAAATCCAGTGACAAGTCTG |
| TCGA-59 | 161 | GAGATCATGTCCTAACCCTGATCCTCTTGTCCCACAGATATCCAGAACCCTCTAAGAAGTTCCTGCTtttaTCGAtattaaatatattaaGAATGTGGTTAGAGACAACGGTTTGACCCTGCCGTGTAC |
| | | CAGCTGAGAGACTCTAAATCCAGTGACAAGTCTG |
| TCGA-60 | 162 | GAGATCATGTCCTAACCCTGATCCTCTTGTCCCACAGATATCCAGAACCCTCTAAGAAGTTCCTGCTaatttaTCGAtattaaatatattGAATGTGGTTAGAGACAACGTAGTGACCCTGCCGTGTAC |
| | | CAGCTGAGAGACTCTAAATCCAGTGACAAGTCTG |
| TCGA-61 | 163 | GAGATCATGTCCTAACCCTGATCCTCTTGTCCCACAGATATCCAGAACCCTCTAAGAAGTTCCTGCTttaatttaTCGAtattaaatataGAATGTGGTTAGAGACAACGTCTTGACCCTGCCGTGTAC |
| | | CAGCTGAGAGACTCTAAATCCAGTGACAAGTCTG |
| TCGA-62 | 164 | GAGATCATGTCCTAACCCTGATCCTCTTGTCCCACAGATATCCAGAACCCTCTAAGAAGTTCCTGCTtattaatttaTCGAtattaaataGAATGTGGTTAGAGACAACGTTATGACCCTGCCGTGTAC |
| | | CAGCTGAGAGACTCTAAATCCAGTGACAAGTCTG |
| TCGA-63 | 165 | GAGATCATGTCCTAACCCTGATCCTCTTGTCCCACAGATATCCAGAACCCTCTAAGAAGTTCCTGCTtatattaatttaTCGAtattaaaGAATGTGGTTAGAGACAACTACTTGACCCTGCCGTGTAC |
| | | CAGCTGAGAGACTCTAAATCCAGTGACAAGTCTG |
| TCGA-64 | 166 | GAGATCATGTCCTAACCCTGATCCTCTTGTCCCACAGATATCCAGAACCCTCTAAGAAGTTCCTGCTaatatattaatttaTCGAtattaGAATGTGGTTAGAGACAACTCAATGACCCTGCCGTGTAC |
| | | CAGCTGAGAGACTCTAAATCCAGTGACAAGTCTG |
| TCGA-65 | 167 | GAGATCATGTCCTAACCCTGATCCTCTTGTCCCACAGATATCCAGAACCCTCTAAGAAGTTCCTGCTtaaatatattaatttaTCGAtatGAATGTGGTTAGAGACAACTCTGTGACCCTGCCGTGTAC |
| | | CAGCTGAGAGACTCTAAATCCAGTGACAAGTCTG |
| TCGA-66 | 168 | GAGATCATGTCCTAACCCTGATCCTCTTGTCCCACAGATATCCAGAACCCTCTAAGAAGTTCCTGCTattaaatatattaatttaTCGAtGAATGTGGTTAGAGACAACTGAATGACCCTGCCGTGTAC |
| | | CAGCTGAGAGACTCTAAATCCAGTGACAAGTCTG |
| TCGA-67 | 169 | GAGATCATGTCCTAACCCTGATCCTCTTGTCCCACAGATATCCAGAACCCTCTAAGAAGTTCCTGCTAtattaaatatattaatttaTCGGAATGTGGTTAGAGACAACTGATTGACCCTGCCGTGTAC |
| | | CAGCTGAGAGACTCTAAATCCAGTGACAAGTCTG |
| TCGA-68 | 170 | GAGATCATGTCCTAACCCTGATCCTCTTGTCCCACAGATATCCAGAACCCTCTAAGAAGTTCCTGCTTCGAtaattaatatattaataaataGAATGTGGTTAGAGACAACTGGATGACCCTGCCGTGT |
| | | ACCAGCTGAGAGACTCTAAATCCAGTGACAAGTCTG |
| TCGA-69 | 171 | GAGATCATGTCCTAACCCTGATCCTCTTGTCCCACAGATATCCAGAACCCTCTAAGAAGTTCCTGCTtaTCGAtaattaatatattaataaaGAATGTGGTTAGAGACAACTGTCTGACCCTGCCGTGT |
| | | ACCAGCTGAGAGACTCTAAATCCAGTGACAAGTCTG |
| TCGA-70 | 172 | GAGATCATGTCCTAACCCTGATCCTCTTGTCCCACAGATATCCAGAACCCTCTAAGAAGTTCCTGCTaataTCGAtaattaatatattaataGAATGTGGTTAGAGACAACTTGTTGACCCTGCCGTGTA |
| | | CCAGCTGAGAGACTCTAAATCCAGTGACAAGTCTG |
| TCGA-71 | 173 | GAGATCATGTCCTAACCCTGATCCTCTTGTCCCACAGATATCCAGAACCCTCTAAGAAGTTCCTGCTtaaataTCGAtaattaatatattaaGAATGTGGTTAGAGACAAGAACCTGACCCTGCCGTGTA |
| | | CCAGCTGAGAGACTCTAAATCCAGTGACAAGTCTG |
| TCGA-72 | 174 | GAGATCATGTCCTAACCCTGATCCTCTTGTCCCACAGATATCCAGAACCCTCTAAGAAGTTCCTGCTaataaataTCGAtaattaatatattGAATGTGGTTAGAGACAAGAACGTGACCCTGCCGTGTA |
| | | CCAGCTGAGAGACTCTAAATCCAGTGACAAGTCTG |
| TCGA-73 | 175 | GAGATCATGTCCTAACCCTGATCCTCTTGTCCCACAGATATCCAGAACCCTCTAAGAAGTTCCTGCTttaataaataTCGAtaattaatataGAATGTGGTTAGAGACAAGAAGATGACCCTGCCGTGTA |
| | | CCAGCTGAGAGACTCTAAATCCAGTGACAAGTCTG |
| TCGA-74 | 176 | GAGATCATGTCCTAACCCTGATCCTCTTGTCCCACAGATATCCAGAACCCTCTAAGAAGTTCCTGCTtattaataaataTCGAtaattaataGAATGTGGTTAGAGACAAGAAGTTGACCCTGCCGTGTA |
| | | CCAGCTGAGAGACTCTAAATCCAGTGACAAGTCTG |
| TCGA-75 | 177 | GAGATCATGTCCTAACCCTGATCCTCTTGTCCCACAGATATCCAGAACCCTCTAAGAAGTTCCTGCTtatattaataaataTCGAtaattaaGAATGTGGTTAGAGACAAGAATCTGACCCTGCCGTGTA |
| | | CCAGCTGAGAGACTCTAAATCCAGTGACAAGTCTG |
| TCGA-76 | 178 | GAGATCATGTCCTAACCCTGATCCTCTTGTCCCACAGATATCCAGAACCCTCTAAGAAGTTCCTGCTaatatattaataaataTCGAtaattGAATGTGGTTAGAGACAAGACTGTGACCCTGCCGTGTA |
| | | CCAGCTGAGAGACTCTAAATCCAGTGACAAGTCTG |
| TCGA-77 | 179 | GAGATCATGTCCTAACCCTGATCCTCTTGTCCCACAGATATCCAGAACCCTCTAAGAAGTTCCTGCTttaatatattaataaataTCGAtaaGAATGTGGTTAGAGACAAGAGAATGACCCTGCCGTGTA |
| | | CCAGCTGAGAGACTCTAAATCCAGTGACAAGTCTG |
| TCGA-78 | 180 | GAGATCATGTCCTAACCCTGATCCTCTTGTCCCACAGATATCCAGAACCCTCTAAGAAGTTCCTGCTaattaatatattaataaataTCGAtGAATGTGGTTAGAGACAAGAGAGTGACCCTGCCGTGTA |
| | | CCAGCTGAGAGACTCTAAATCCAGTGACAAGTCTG |
| TCGA-79 | 181 | GAGATCATGTCCTAACCCTGATCCTCTTGTCCCACAGATATCCAGAACCCTCTAAGAAGTTCCTGCTAtaattaatatattaataaataTCGGAATGTGGTTAGAGACAAGAGCATGACCCTGCCGTGTA |
| | | CCAGCTGAGAGACTCTAAATCCAGTGACAAGTCTG |
| TCGA-80 | 182 | GAGATCATGTCCTAACCCTGATCCTCTTGTCCCACAGATATCCAGAACCCTCTAAGAAGTTCCTGCTTCGAttattaatatataaataattataGAATGTGGTTAGAGACAAGAGCTTGACCCTGCCGTG |
| | | CTACAGCTGAGAGACTCTAAATCCAGTGACAAGTCTG |
| TCGA-81 | 183 | GAGATCATGTCCTAACCCTGATCCTCTTGTCCCACAGATATCCAGAACCCTCTAAGAAGTTCCTGCTtaTCGAttattaatatataaataattaGAATGTGGTTAGAGACAAGAGGATGACCCTGCCGTG |
| | | TACCAGCTGAGAGACTCTAAATCCAGTGACAAGTCTG |
| TCGA-82 | 184 | GAGATCATGTCCTAACCCTGATCCTCTTGTCCCACAGATATCCAGAACCCTCTAAGAAGTTCCTGCTtataTCGAttattaatatataaataatGAATGTGGTTAGAGACAAGATACTGACCCTGCCGTG |
| | | TACCAGCTGAGAGACTCTAAATCCAGTGACAAGTCTG |
| TCGA-83 | 185 | GAGATCATGTCCTAACCCTGATCCTCTTGTCCCACAGATATCCAGAACCCTCTAAGAAGTTCCTGCTattataTCGAttattaatatataaataGAATGTGGTTAGAGACAAGATGCTGACCCTGCCGTG |
| | | TACCAGCTGAGAGACTCTAAATCCAGTGACAAGTCTG |
| TCGA-84 | 186 | GAGATCATGTCCTAACCCTGATCCTCTTGTCCCACAGATATCCAGAACCCTCTAAGAAGTTCCTGCTtaattataTCGAttattaatatataaaGAATGTGGTTAGAGACAAGATGGTGACCCTGCCGTG |
| | | TACCAGCTGAGAGACTCTAAATCCAGTGACAAGTCTG |
| TCGA-85 | 187 | GAGATCATGTCCTAACCCTGATCCTCTTGTCCCACAGATATCCAGAACCCTCTAAGAAGTTCCTGCTaataattataTCGAttattaatatataGAATGTGGTTAGAGACAAGCAAATGACCCTGCCGTG |
| | | TACCAGCTGAGAGACTCTAAATCCAGTGACAAGTCTG |
| TCGA-86 | 188 | GAGATCATGTCCTAACCCTGATCCTCTTGTCCCACAGATATCCAGAACCCTCTAAGAAGTTCCTGCTtaaataattataTCGAttattaatataGAATGTGGTTAGAGACAAGCACTTGACCCTGCCGTG |
| | | TACCAGCTGAGAGACTCTAAATCCAGTGACAAGTCTG |
| TCGA-87 | 189 | GAGATCATGTCCTAACCCTGATCCTCTTGTCCCACAGATATCCAGAACCCTCTAAGAAGTTCCTGCTtataaataattataTCGAttattaataGAATGTGGTTAGAGACAAGCATGTGACCCTGCCGTG |
| | | TACCAGCTGAGAGACTCTAAATCCAGTGACAAGTCTG |
| TCGA-88 | 190 | GAGATCATGTCCTAACCCTGATCCTCTTGTCCCACAGATATCCAGAACCCTCTAAGAAGTTCCTGCTtatataaataattataTCGAttattaaGAATGTGGTTAGAGACAAGCCTTTGACCCTGCCGTG |
| | | TACCAGCTGAGAGACTCTAAATCCAGTGACAAGTCTG |
| TCGA-89 | 191 | GAGATCATGTCCTAACCCTGATCCTCTTGTCCCACAGATATCCAGAACCCTCTAAGAAGTTCCTGCTaatatataaataattataTCGAttattGAATGTGGTTAGAGACAAGCGATTGACCCTGCCGTG |
| | | TACCAGCTGAGAGACTCTAAATCCAGTGACAAGTCTG |
| TCGA-90 | 192 | GAGATCATGTCCTAACCCTGATCCTCTTGTCCCACAGATATCCAGAACCCTCTAAGAAGTTCCTGCTttaatatataaataattataTCGAttaGAATGTGGTTAGAGACAAGCTCATGACCCTGCCGTG |
| | | TACCAGCTGAGAGACTCTAAATCCAGTGACAAGTCTG |
| TCGA-91 | 193 | GAGATCATGTCCTAACCCTGATCCTCTTGTCCCACAGATATCCAGAACCCTCTAAGAAGTTCCTGCTtattaatatataaataattataTCGAtGAATGTGGTTAGAGACAAGCTTATGACCCTGCCGTG |
| | | TACCAGCTGAGAGACTCTAAATCCAGTGACAAGTCTG |
| TCGA-92 | 194 | GAGATCATGTCCTAACCCTGATCCTCTTGTCCCACAGATATCCAGAACCCTCTAAGAAGTTCCTGCTAttattaatatataaataattataTCGGAATGTGGTTAGAGACAAGGAAATGACCCTGCCGTG |
| | | TACCAGCTGAGAGACTCTAAATCCAGTGACAAGTCTG |
| TCGA-93 | 195 | GAGATCATGTCCTAACCCTGATCCTCTTGTCCCACAGATATCCAGAACCCTCTAAGAAGTTCCTGCTTCGAttattaatatataaatatttaaataGAATGTGGTTAGAGACAAGGAAGTGACCCTGCCG |
| | | TGTACCAGCTGAGAGACTCTAAATCCAGTGACAAGTCTG |
| TCGA-94 | 196 | GAGATCATGTCCTAACCCTGATCCTCTTGTCCCACAGATATCCAGAACCCTCTAAGAAGTTCCTGCTtaTCGAttattaatatataaatatttaaaGAATGTGGTTAGAGACAAGGGAATGACCCTGCCG |
| | | TGTACCAGCTGAGAGACTCTAAATCCAGTGACAAGTCTG |
| TCGA-95 | 197 | GAGATCATGTCCTAACCCTGATCCTCTTGTCCCACAGATATCCAGAACCCTCTAAGAAGTTCCTGCTaataTCGAttattaatatataaatatttaGAATGTGGTTAGAGACAAGGTACTGACCCTGCCG |
| | | TGTACCAGCTGAGAGACTCTAAATCCAGTGACAAGTCTG |
| TCGA-96 | 198 | GAGATCATGTCCTAACCCTGATCCTCTTGTCCCACAGATATCCAGAACCCTCTAAGAAGTTCCTGCTtaaataTCGAttattaatatataaatattGAATGTGGTTAGAGACAAGGTTATGACCCTGCCG |
| | | TGTACCAGCTGAGAGACTCTAAATCCAGTGACAAGTCTG |
| TCGA-97 | 199 | GAGATCATGTCCTAACCCTGATCCTCTTGTCCCACAGATATCCAGAACCCTCTAAGAAGTTCCTGCTtttaaataTCGAttattaatatataaataGAATGTGGTTAGAGACAAGGTTGTGACCCTGCCG |
| | | TGTACCAGCTGAGAGACTCTAAATCCAGTGACAAGTCTG |
| TCGA-98 | 200 | GAGATCATGTCCTAACCCTGATCCTCTTGTCCCACAGATATCCAGAACCCTCTAAGAAGTTCCTGCTtatttaaataTCGAttattaatatataaaGAATGTGGTTAGAGACAAGTAACTGACCCTGCCG |
| | | TGTACCAGCTGAGAGACTCTAAATCCAGTGACAAGTCTG |
| TCGA-99 | 201 | GAGATCATGTCCTAACCCTGATCCTCTTGTCCCACAGATATCCAGAACCCTCTAAGAAGTTCCTGCTaatatttaaataTCGAttattaatatataGAATGTGGTTAGAGACAAGTACATGACCCTGCCG |
| | | TGTACCAGCTGAGAGACTCTAAATCCAGTGACAAGTCTG |
| TCGA- | 202 | GAGATCATGTCCTAACCCTGATCCTCTTGTCCCACAGATATCCAGAACCCTCTAAGAAGTTCCTGCTtaaatatttaaataTCGAttattaatataGAATGTGGTTAGAGACAAGTACGTGACCCTGCCG |
| 100 | | TGTACCAGCTGAGAGACTCTAAATCCAGTGACAAGTCTG |
| TCGA- | 203 | GAGATCATGTCCTAACCCTGATCCTCTTGTCCCACAGATATCCAGAACCCTCTAAGAAGTTCCTGCTtataaatatttaaataTCGAttattaataGAATGTGGTTAGAGACAAGTAGCTGACCCTGCCG |
| 101 | | TGTACCAGCTGAGAGACTCTAAATCCAGTGACAAGTCTG |
| TCGA- | 204 | GAGATCATGTCCTAACCCTGATCCTCTTGTCCCACAGATATCCAGAACCCTCTAAGAAGTTCCTGCTtatataaatatttaaataTCGAttattaaGAATGTGGTTAGAGACAAGTCTCTGACCCTGCCG |
| 102 | | TGTACCAGCTGAGAGACTCTAAATCCAGTGACAAGTCTG |
| TCGA- | 205 | GAGATCATGTCCTAACCCTGATCCTCTTGTCCCACAGATATCCAGAACCCTCTAAGAAGTTCCTGCTaatatataaatatttaaataTCGAttattGAATGTGGTTAGAGACAAGTCTGTGACCCTGCCG |
| 103 | | TGTACCAGCTGAGAGACTCTAAATCCAGTGACAAGTCTG |
| TCGA- | 206 | GAGATCATGTCCTAACCCTGATCCTCTTGTCCCACAGATATCCAGAACCCTCTAAGAAGTTCCTGCTttaatatataaatatttaaataTCGAttaGAATGTGGTTAGAGACAAGTGTGTGACCCTGCCG |
| 104 | | TGTACCAGCTGAGAGACTCTAAATCCAGTGACAAGTCTG |
| TCGA- | 207 | GAGATCATGTCCTAACCCTGATCCTCTTGTCCCACAGATATCCAGAACCCTCTAAGAAGTTCCTGCTtattaatatataaatatttaaataTCGAtGAATGTGGTTAGAGACAAGTTAGTGACCCTGCC |
| 105 | | GTGTACCAGCTGAGAGACTCTAAATCCAGTGACAAGTCTG |
| TCGA- | 208 | GAGATCATGTCCTAACCCTGATCCTCTTGTCCCACAGATATCCAGAACCCTCTAAGAAGTTCCTGCTAttattaatatataaatatttaaataTCGGAATGTGGTTAGAGACAAGTTCCTGACCCTGCC |
| 106 | | GTGTACCAGCTGAGAGACTCTAAATCCAGTGACAAGTCTG |
| TCGA- | 209 | GAGATCATGTCCTAACCCTGATCCTCTTGTCCCACAGATATCCAGAACCCTCTAAGAAGTTCCTGCTTCGAttattaattaataaatatttaaatataGAATGTGGTTAGAGACAAGTTCTTGACCCTG |
| 107 | | CCGTGTACCAGCTGAGAGACTCTAAATCCAGTGACAAGTCTG |
| TCGA- | 210 | GAGATCATGTCCTAACCCTGATCCTCTTGTCCCACAGATATCCAGAACCCTCTAAGAAGTTCCTGCTtaTCGAttattaattaataaatatttaaataGAATGTGGTTAGAGACAATAAGCTGACCCTGC |
| 108 | | CGTGTACCAGCTGAGAGACTCTAAATCCAGTGACAAGTCTG |
| TCGA- | 211 | GAGATCATGTCCTAACCCTGATCCTCTTGTCCCACAGATATCCAGAACCCTCTAAGAAGTTCCTGCTtataTCGAttattaattaataaatatttaaaGAATGTGGTTAGAGACAATACTCTGACCCTGC |
| 109 | | CGTGTACCAGCTGAGAGACTCTAAATCCAGTGACAAGTCTG |
| TCGA- | 212 | GAGATCATGTCCTAACCCTGATCCTCTTGTCCCACAGATATCCAGAACCCTCTAAGAAGTTCCTGCTaatataTCGAttattaattaataaatatttaGAATGTGGTTAGAGACAATCACATGACCCTGC |
| 110 | | CGTGTACCAGCTGAGAGACTCTAAATCCAGTGACAAGTCTG |
| TCGA- | 213 | GAGATCATGTCCTAACCCTGATCCTCTTGTCCCACAGATATCCAGAACCCTCTAAGAAGTTCCTGCTtaaatataTCGAttattaattaataaatattGAATGTGGTTAGAGACAATCATCTGACCCTGC |
| 111 | | CGTGTACCAGCTGAGAGACTCTAAATCCAGTGACAAGTCTG |
| TCGA- | 214 | GAGATCATGTCCTAACCCTGATCCTCTTGTCCCACAGATATCCAGAACCCTCTAAGAAGTTCCTGCTtttaaatataTCGAttattaattaataaataGAATGTGGTTAGAGACAATCCCTTGACCCTGC |
| 112 | | CGTGTACCAGCTGAGAGACTCTAAATCCAGTGACAAGTCTG |
| TCGA- | 215 | GAGATCATGTCCTAACCCTGATCCTCTTGTCCCACAGATATCCAGAACCCTCTAAGAAGTTCCTGCTtatttaaatataTCGAttattaattaataaaGAATGTGGTTAGAGACAATCCGATGACCCTGC |
| 113 | | CGTGTACCAGCTGAGAGACTCTAAATCCAGTGACAAGTCTG |
| TCGA- | 216 | GAGATCATGTCCTAACCCTGATCCTCTTGTCCCACAGATATCCAGAACCCTCTAAGAAGTTCCTGCTaatatttaaatataTCGAttattaattaataGAATGTGGTTAGAGACAATCGAATGACCCTGC |
| 114 | | CGTGTACCAGCTGAGAGACTCTAAATCCAGTGACAAGTCTG |
| TCGA- | 217 | GAGATCATGTCCTAACCCTGATCCTCTTGTCCCACAGATATCCAGAACCCTCTAAGAAGTTCCTGCTtaaatatttaaatataTCGAttattaattaaGAATGTGGTTAGAGACAATCGGATGACCCTGC |
| 115 | | CGTGTACCAGCTGAGAGACTCTAAATCCAGTGACAAGTCTG |
| TCGA- | 218 | GAGATCATGTCCTAACCCTGATCCTCTTGTCCCACAGATATCCAGAACCCTCTAAGAAGTTCCTGCTaataaatatttaaatataTCGAttattaattGAATGTGGTTAGAGACAATCGGTTGACCCTGC |
| 116 | | CGTGTACCAGCTGAGAGACTCTAAATCCAGTGACAAGTCTG |
| TCGA- | 219 | GAGATCATGTCCTAACCCTGATCCTCTTGTCCCACAGATATCCAGAACCCTCTAAGAAGTTCCTGCTttaataaatatttaaatataTCGAttattaaGAATGTGGTTAGAGACAATCGTCTGACCCTGC |
| 117 | | CGTGTACCAGCTGAGAGACTCTAAATCCAGTGACAAGTCTG |
| TCGA- | 220 | GAGATCATGTCCTAACCCTGATCCTCTTGTCCCACAGATATCCAGAACCCTCTAAGAAGTTCCTGCTaattaataaatatttaaatataTCGAttattGAATGTGGTTAGAGACAATCGTGTGACCCTGC |
| 118 | | CGTGTACCAGCTGAGAGACTCTAAATCCAGTGACAAGTCTG |
| TCGA- | 221 | GAGATCATGTCCTAACCCTGATCCTCTTGTCCCACAGATATCCAGAACCCTCTAAGAAGTTCCTGCTttaattaataaatatttaaatataTCGAttaGAATGTGGTTAGAGACAATGACCTGACCCTGC |
| 119 | | CGTGTACCAGCTGAGAGACTCTAAATCCAGTGACAAGTCTG |
| TCGA- | 222 | GAGATCATGTCCTAACCCTGATCCTCTTGTCCCACAGATATCCAGAACCCTCTAAGAAGTTCCTGCTtattaattaataaatatttaaatataTCGAtGAATGTGGTTAGAGACAATGACGTGACCCTGC |
| 120 | | CGTGTACCAGCTGAGAGACTCTAAATCCAGTGACAAGTCTG |
| TCGA- | 223 | GAGATCATGTCCTAACCCTGATCCTCTTGTCCCACAGATATCCAGAACCCTCTAAGAAGTTCCTGCTAttattaattaataaatatttaaatataTCGGAATGTGGTTAGAGACAATGCCATGACCCTGC |
| 121 | | CGTGTACCAGCTGAGAGACTCTAAATCCAGTGACAAGTCTG |
| TCGA- | 224 | GAGATCATGTCCTAACCCTGATCCTCTTGTCCCACAGATATCCAGAACCCTCTAAGAAGTTCCTGCTTCGAttattaattaataaatatttatttaaataGAATGTGGTTAGAGACAATGCTATGACCCT |
| 122 | | GCCGTGTACCAGCTGAGAGACTCTAAATCCAGTGACAAGTCTG |
| TCGA- | 225 | GAGATCATGTCCTAACCCTGATCCTCTTGTCCCACAGATATCCAGAACCCTCTAAGAAGTTCCTGCTtaTCGAttattaattaataaatatttatttaaaGAATGTGGTTAGAGACAATGCTTTGACCCT |
| 123 | | GCCGTGTACCAGCTGAGAGACTCTAAATCCAGTGACAAGTCTG |
| TCGA- | 226 | GAGATCATGTCCTAACCCTGATCCTCTTGTCCCACAGATATCCAGAACCCTCTAAGAAGTTCCTGCTaataTCGAttattaattaataaatatttatttaGAATGTGGTTAGAGACAATGGACTGACCCT |
| 124 | | GCCGTGTACCAGCTGAGAGACTCTAAATCCAGTGACAAGTCTG |
| TCGA- | 227 | GAGATCATGTCCTAACCCTGATCCTCTTGTCCCACAGATATCCAGAACCCTCTAAGAAGTTCCTGCTtaaataTCGAttattaattaataaatatttattGAATGTGGTTAGAGACAATTCACTGACCCT |
| 125 | | GCCGTGTACCAGCTGAGAGACTCTAAATCCAGTGACAAGTCTG |
| TCGA- | 228 | GAGATCATGTCCTAACCCTGATCCTCTTGTCCCACAGATATCCAGAACCCTCTAAGAAGTTCCTGCTtttaaataTCGAttattaattaataaatatttaGAATGTGGTTAGAGACAATTCCGTGACCCT |
| 126 | | GCCGTGTACCAGCTGAGAGACTCTAAATCCAGTGACAAGTCTG |
| TCGA- | 229 | GAGATCATGTCCTAACCCTGATCCTCTTGTCCCACAGATATCCAGAACCCTCTAAGAAGTTCCTGCTtatttaaataTCGAttattaattaataaatattGAATGTGGTTAGAGACAATTCGATGACCC |
| 127 | | TGCCGTGTACCAGCTGAGAGACTCTAAATCCAGTGACAAGTCTG |
| TCGA- | 230 | GAGATCATGTCCTAACCCTGATCCTCTTGTCCCACAGATATCCAGAACCCTCTAAGAAGTTCCTGCTtttatttaaataTCGAttattaattaataaataGAATGTGGTTAGAGACAATTGCATGACCC |
| 128 | | TGCCGTGTACCAGCTGAGAGACTCTAAATCCAGTGACAAGTCTG |
| TCGA- | 231 | GAGATCATGTCCTAACCCTGATCCTCTTGTCCCACAGATATCCAGAACCCTCTAAGAAGTTCCTGCTtatttatttaaataTCGAttattaattaataaaGAATGTGGTTAGAGACAATTGCTTGACCCT |
| 129 | | GCCGTGTACCAGCTGAGAGACTCTAAATCCAGTGACAAGTCTG |
| TCGA- | 232 | GAGATCATGTCCTAACCCTGATCCTCTTGTCCCACAGATATCCAGAACCCTCTAAGAAGTTCCTGCTaatatttatttaaataTCGAttattaattaataGAATGTGGTTAGAGACACAAATGTGACCCT |
| 130 | | GCCGTGTACCAGCTGAGAGACTCTAAATCCAGTGACAAGTCTG |
| TCGA- | 233 | GAGATCATGTCCTAACCCTGATCCTCTTGTCCCACAGATATCCAGAACCCTCTAAGAAGTTCCTGCTtaaatatttatttaaataTCGAttattaattaaGAATGTGGTTAGAGACACAACTTTGACCCT |
| 131 | | GCCGTGTACCAGCTGAGAGACTCTAAATCCAGTGACAAGTCTG |
| TCGA- | 234 | GAGATCATGTCCTAACCCTGATCCTCTTGTCCCACAGATATCCAGAACCCTCTAAGAAGTTCCTGCTaataaatatttatttaaataTCGAttattaattGAATGTGGTTAGAGACACAAGATTGACCCT |
| 132 | | GCCGTGTACCAGCTGAGAGACTCTAAATCCAGTGACAAGTCTG |
| TCGA- | 235 | GAGATCATGTCCTAACCCTGATCCTCTTGTCCCACAGATATCCAGAACCCTCTAAGAAGTTCCTGCTttaataaatatttatttaaataTCGAttattaaGAATGTGGTTAGAGACACAAGGTTGACCCT |
| 133 | | GCCGTGTACCAGCTGAGAGACTCTAAATCCAGTGACAAGTCTG |
| TCGA- | 236 | GAGATCATGTCCTAACCCTGATCCTCTTGTCCCACAGATATCCAGAACCCTCTAAGAAGTTCCTGCTaattaataaatatttatttaaataTCGAttattGAATGTGGTTAGAGACACAATTGTGACCCT |
| 134 | | GCCGTGTACCAGCTGAGAGACTCTAAATCCAGTGACAAGTCTG |
| TCGA- | 237 | GAGATCATGTCCTAACCCTGATCCTCTTGTCCCACAGATATCCAGAACCCTCTAAGAAGTTCCTGCTttaattaataaatatttatttaaataTCGAttaGAATGTGGTTAGAGACACACACTTGACCCT |
| 135 | | GCCGTGTACCAGCTGAGAGACTCTAAATCCAGTGACAAGTCTG |
| TCGA | 238 | GAGATCATGTCCTAACCCTGATCCTCTTGTCCCACAGATATCCAGAACCCTCTAAGAAGTTCCTGCTtattaattaataaatatttatttaaataTCGAtGAATGTGGTTAGAGACACACATATGACCC |
| 136 | | TGCCGTGTACCAGCTGAGAGACTCTAAATCCAGTGACAAGTCTG |
| TCGA- | 239 | GAGATCATGTCCTAACCCTGATCCTCTTGTCCCACAGATATCCAGAACCCTCTAAGAAGTTCCTGCTAttattaattaataaatatttatttaaataTCGGAATGTGGTTAGAGACACACGTATGACCC |
| 137 | | TGCCGTGTACCAGCTGAGAGACTCTAAATCCAGTGACAAGTCTG |
| TCGA- | 240 | GAGATCATGTCCTAACCCTGATCCTCTTGTCCCACAGATATCCAGAACCCTCTAAGAAGTTCCTGCTTCGAttattaatattaataaatatttatttaaataGAATGTGGTTAGAGACACACTAATGACC |
| 138 | | CTGCCGTGTACCAGCTGAGAGACTCTAAATCCAGTGACAAGTCTG |
| TCGA- | 241 | GAGATCATGTCCTAACCCTGATCCTCTTGTCCCACAGATATCCAGAACCCTCTAAGAAGTTCCTGCTtaTCGAttattaatattaataaatatttatttaaaGAATGTGGTTAGAGACACACTAGTGACC |
| 139 | | CTGCCGTGTACCAGCTGAGAGACTCTAAATCCAGTGACAAGTCTG |
| TCGA- | 242 | GAGATCATGTCCTAACCCTGATCCTCTTGTCCCACAGATATCCAGAACCCTCTAAGAAGTTCCTGCTaataTCGAttattaatattaataaatatttatttaGAATGTGGTTAGAGACACACTCTTGACC |
| 140 | | CTGCCGTGTACCAGCTGAGAGACTCTAAATCCAGTGACAAGTCTG |
| TCGA- | 243 | GAGATCATGTCCTAACCCTGATCCTCTTGTCCCACAGATATCCAGAACCCTCTAAGAAGTTCCTGCTtaaataTCGAttattaatattaataaatatttattGAATGTGGTTAGAGACACAGCAATGAC |
| 141 | | CCTGCCGTGTACCAGCTGAGAGACTCTAAATCCAGTGACAAGTCTG |
| TCGA- | 244 | GAGATCATGTCCTAACCCTGATCCTCTTGTCCCACAGATATCCAGAACCCTCTAAGAAGTTCCTGCTtttaaataTCGAttattaatattaataaatatttaGAATGTGGTTAGAGACACAGCATTGACC |
| 142 | | CTGCCGTGTACCAGCTGAGAGACTCTAAATCCAGTGACAAGTCTG |
| TCGA- | 245 | GAGATCATGTCCTAACCCTGATCCTCTTGTCCCACAGATATCCAGAACCCTCTAAGAAGTTCCTGCTtatttaaataTCGAttattaatattaataaatattGAATGTGGTTAGAGACACAGTATTGACC |
| 143 | | CTGCCGTGTACCAGCTGAGAGACTCTAAATCCAGTGACAAGTCTG |
| TCGA- | 246 | GAGATCATGTCCTAACCCTGATCCTCTTGTCCCACAGATATCCAGAACCCTCTAAGAAGTTCCTGCTtttatttaaataTCGAttattaatattaataaataGAATGTGGTTAGAGACACAGTCATGACC |
| 144 | | CTGCCGTGTACCAGCTGAGAGACTCTAAATCCAGTGACAAGTCTG |
| TCGA- | 247 | GAGATCATGTCCTAACCCTGATCCTCTTGTCCCACAGATATCCAGAACCCTCTAAGAAGTTCCTGCTtatttatttaaataTCGAttattaatattaataaaGAATGTGGTTAGAGACACAGTGATGACC |
| 145 | | CTGCCGTGTACCAGCTGAGAGACTCTAAATCCAGTGACAAGTCTG |
| TCGA- | 248 | GAGATCATGTCCTAACCCTGATCCTCTTGTCCCACAGATATCCAGAACCCTCTAAGAAGTTCCTGCTaatatttatttaaataTCGAttattaatattaataGAATGTGGTTAGAGACACAGTGTTGACC |
| 146 | | CTGCCGTGTACCAGCTGAGAGACTCTAAATCCAGTGACAAGTCTG |
| TCGA- | 249 | GAGATCATGTCCTAACCCTGATCCTCTTGTCCCACAGATATCCAGAACCCTCTAAGAAGTTCCTGCTtaaatatttatttaaataTCGAttattaatattaaGAATGTGGTTAGAGACACAGTTCTGACC |
| 147 | | CTGCCGTGTACCAGCTGAGAGACTCTAAATCCAGTGACAAGTCTG |
| TCGA- | 250 | GAGATCATGTCCTAACCCTGATCCTCTTGTCCCACAGATATCCAGAACCCTCTAAGAAGTTCCTGCTaataaatatttatttaaataTCGAttattaatattGAATGTGGTTAGAGACACATAAGTGAC |
| 148 | | CCTGCCGTGTACCAGCTGAGAGACTCTAAATCCAGTGACAAGTCTG |
| TCGA- | 251 | GAGATCATGTCCTAACCCTGATCCTCTTGTCCCACAGATATCCAGAACCCTCTAAGAAGTTCCTGCTttaataaatatttatttaaataTCGAttattaataGAATGTGGTTAGAGACACATATGTGAC |
| 149 | | CCTGCCGTGTACCAGCTGAGAGACTCTAAATCCAGTGACAAGTCTG |
| TCGA- | 252 | GAGATCATGTCCTAACCCTGATCCTCTTGTCCCACAGATATCCAGAACCCTCTAAGAAGTTCCTGCTtattaataaatatttatttaaataTCGAttattaaGAATGTGGTTAGAGACACATCACTGACC |
| 150 | | CTGCCGTGTACCAGCTGAGAGACTCTAAATCCAGTGACAAGTCTG |
| TCGA- | 253 | GAGATCATGTCCTAACCCTGATCCTCTTGTCCCACAGATATCCAGAACCCTCTAAGAAGTTCCTGCTaatattaataaatatttatttaaataTCGAttattGAATGTGGTTAGAGACACATCCTTGACC |
| 151 | | CTGCCGTGTACCAGCTGAGAGACTCTAAATCCAGTGACAAGTCTG |
| TCGA- | 254 | GAGATCATGTCCTAACCCTGATCCTCTTGTCCCACAGATATCCAGAACCCTCTAAGAAGTTCCTGCTttaatattaataaatatttatttaaataTCGAttaGAATGTGGTTAGAGACACATCGTTGAC |
| 152 | | CCTGCCGTGTACCAGCTGAGAGACTCTAAATCCAGTGACAAGTCTG |
| TCGA- | 255 | GAGATCATGTCCTAACCCTGATCCTCTTGTCCCACAGATATCCAGAACCCTCTAAGAAGTTCCTGCTtattaatattaataaatatttatttaaataTCGAtGAATGTGGTTAGAGACACATGACTGAC |
| 153 | | CCTGCCGTGTACCAGCTGAGAGACTCTAAATCCAGTGACAAGTCTG |
| TCGA- | 256 | GAGATCATGTCCTAACCCTGATCCTCTTGTCCCACAGATATCCAGAACCCTCTAAGAAGTTCCTGCTAttattaatattaataaatatttatttaaataTCGGAATGTGGTTAGAGACACATGTATG |
| 154 | | ACCCTGCCGTGTACCAGCTGAGAGACTCTAAATCCAGTGACAAGTCTG |
| TCGA- | 257 | GAGATCATGTCCTAACCCTGATCCTCTTGTCCCACAGATATCCAGAACCCTCTAAGAAGTTCCTGCTTCGAttaattaatattaataaatatttatttataataGAATGTGGTTAGAGACACATGTGTG |
| 155 | | ACCCTGCCGTGTACCAGCTGAGAGACTCTAAATCCAGTGACAAGTCTG |
| TCGA- | 258 | GAGATCATGTCCTAACCCTGATCCTCTTGTCCCACAGATATCCAGAACCCTCTAAGAAGTTCCTGCTtaTCGAttaattaatattaataaatatttatttataaGAATGTGGTTAGAGACACATTCTTG |
| 156 | | ACCCTGCCGTGTACCAGCTGAGAGACTCTAAATCCAGTGACAAGTCTG |
| TCGA- | 259 | GAGATCATGTCCTAACCCTGATCCTCTTGTCCCACAGATATCCAGAACCCTCTAAGAAGTTCCTGCTaataTCGAttaattaatattaataaatatttatttatGAATGTGGTTAGAGACACCAAACT |
| 157 | | GACCCTGCCGTGTACCAGCTGAGAGACTCTAAATCCAGTGACAAGTCTG |
| TCGA- | 260 | GAGATCATGTCCTAACCCTGATCCTCTTGTCCCACAGATATCCAGAACCCTCTAAGAAGTTCCTGCTataataTCGAttaattaatattaataaatatttatttGAATGTGGTTAGAGACACCAAAGTG |
| 158 | | ACCCTGCCGTGTACCAGCTGAGAGACTCTAAATCCAGTGACAAGTCTG |
| TCGA- | 261 | GAGATCATGTCCTAACCCTGATCCTCTTGTCCCACAGATATCCAGAACCCTCTAAGAAGTTCCTGCTttataataTCGAttaattaatattaataaatatttatGAATGTGGTTAGAGACACCAATCTG |
| 159 | | ACCCTGCCGTGTACCAGCTGAGAGACTCTAAATCCAGTGACAAGTCTG |
| TCGA- | 262 | GAGATCATGTCCTAACCCTGATCCTCTTGTCCCACAGATATCCAGAACCCTCTAAGAAGTTCCTGCTatttataataTCGAttaattaatattaataaatatttGAATGTGGTTAGAGACACCACATTG |
| 160 | | ACCCTGCCGTGTACCAGCTGAGAGACTCTAAATCCAGTGACAAGTCTG |
| TCGA- | 263 | GAGATCATGTCCTAACCCTGATCCTCTTGTCCCACAGATATCCAGAACCCTCTAAGAAGTTCCTGCTttatttataataTCGAttaattaatattaataaatatGAATGTGGTTAGAGACACCATATTGA |
| 161 | | CCCTGCCGTGTACCAGCTGAGAGACTCTAAATCCAGTGACAAGTCTG |
| TCGA- | 264 | GAGATCATGTCCTAACCCTGATCCTCTTGTCCCACAGATATCCAGAACCCTCTAAGAAGTTCCTGCTatttatttataataTCGAttaattaatattaataaatGAATGTGGTTAGAGACACCTCATTG |
| 162 | | ACCCTGCCGTGTACCAGCTGAGAGACTCTAAATCCAGTGACAAGTCTG |
| TCGA- | 265 | GAGATCATGTCCTAACCCTGATCCTCTTGTCCCACAGATATCCAGAACCCTCTAAGAAGTTCCTGCTatatttatttataataTCGAttaattaatattaataaGAATGTGGTTAGAGACACCTTCATG |
| 163 | | ACCCTGCCGTGTACCAGCTGAGAGACTCTAAATCCAGTGACAAGTCTG |
| TCGA- | 266 | GAGATCATGTCCTAACCCTGATCCTCTTGTCCCACAGATATCCAGAACCCTCTAAGAAGTTCCTGCTaaatatttatttataataTCGAttaattaatattaatGAATGTGGTTAGAGACACCTTGATG |
| 164 | | ACCCTGCCGTGTACCAGCTGAGAGACTCTAAATCCAGTGACAAGTCTG |
| TCGA- | 267 | GAGATCATGTCCTAACCCTGATCCTCTTGTCCCACAGATATCCAGAACCCTCTAAGAAGTTCCTGCTataaatatttatttataataTCGAttaattaatattaGAATGTGGTTAGAGACACCTTTCTG |
| 165 | | ACCCTGCCGTGTACCAGCTGAGAGACTCTAAATCCAGTGACAAGTCTG |
| TCGA- | 268 | GAGATCATGTCCTAACCCTGATCCTCTTGTCCCACAGATATCCAGAACCCTCTAAGAAGTTCCTGCTtaataaatatttatttataataTCGAttaattaatatGAATGTGGTTAGAGACACGAAAGTG |
| 166 | | ACCCTGCCGTGTACCAGCTGAGAGACTCTAAATCCAGTGACAAGTCTG |
| TCGA- | 269 | GAGATCATGTCCTAACCCTGATCCTCTTGTCCCACAGATATCCAGAACCCTCTAAGAAGTTCCTGCTattaataaatatttatttataataTCGAttaattaatGAATGTGGTTAGAGACACGACTATG |
| 167 | | ACCCTGCCGTGTACCAGCTGAGAGACTCTAAATCCAGTGACAAGTCTG |
| TCGA- | 270 | GAGATCATGTCCTAACCCTGATCCTCTTGTCCCACAGATATCCAGAACCCTCTAAGAAGTTCCTGCTatattaataaatatttatttataataTCGAttaattaGAATGTGGTTAGAGACACGAGTATG |
| 168 | | ACCCTGCCGTGTACCAGCTGAGAGACTCTAAATCCAGTGACAAGTCTG |
| TCGA- | 271 | GAGATCATGTCCTAACCCTGATCCTCTTGTCCCACAGATATCCAGAACCCTCTAAGAAGTTCCTGCTtaatattaataaatatttatttataataTCGAttaatGAATGTGGTTAGAGACACGATCTTG |
| 169 | | ACCCTGCCGTGTACCAGCTGAGAGACTCTAAATCCAGTGACAAGTCTG |
| TCGA- | 272 | GAGATCATGTCCTAACCCTGATCCTCTTGTCCCACAGATATCCAGAACCCTCTAAGAAGTTCCTGCTattaatattaataaatatttatttataataTCGAttaGAATGTGGTTAGAGACACGATGTT |
| 170 | | GACCCTGCCGTGTACCAGCTGAGAGACTCTAAATCCAGTGACAAGTCTG |
| TCGA- | 273 | GAGATCATGTCCTAACCCTGATCCTCTTGTCCCACAGATATCCAGAACCCTCTAAGAAGTTCCTGCTtaattaatattaataaatatttatttataataTCGAtGAATGTGGTTAGAGACACGGTATT |
| 171 | | GACCCTGCCGTGTACCAGCTGAGAGACTCTAAATCCAGTGACAAGTCTG |
| TCGA- | 274 | GAGATCATGTCCTAACCCTGATCCTCTTGTCCCACAGATATCCAGAACCCTCTAAGAAGTTCCTGCTAttaattaatattaataaatatttatttataataTCGGAATGTGGTTAGAGACACGGTTTT |
| 172 | | GACCCTGCCGTGTACCAGCTGAGAGACTCTAAATCCAGTGACAAGTCTG |
| TCGA- | 275 | GAGATCATGTCCTAACCCTGATCCTCTTGTCCCACAGATATCCAGAACCCTCTAAGAAGTTCCTGCTTCGAttaattaatattaatttaaatatttatttatataaGAATGTGGTTAGAGACACGTAAT |
| 173 | | TGACCCTGCCGTGTACCAGCTGAGAGACTCTAAATCCAGTGACAAGTCTG |
| TCGA- | 276 | GAGATCATGTCCTAACCCTGATCCTCTTGTCCCACAGATATCCAGAACCCTCTAAGAAGTTCCTGCTaaTCGAttaattaatattaatttaaatatttatttatatGAATGTGGTTAGAGACACGTATT |
| 174 | | TGACCCTGCCGTGTACCAGCTGAGAGACTCTAAATCCAGTGACAAGTCTG |
| TCGA- | 277 | GAGATCATGTCCTAACCCTGATCCTCTTGTCCCACAGATATCCAGAACCCTCTAAGAAGTTCCTGCTataaTCGAttaattaatattaatttaaatatttatttatGAATGTGGTTAGAGACACGTGAA |
| 175 | | TGACCCTGCCGTGTACCAGCTGAGAGACTCTAAATCCAGTGACAAGTCTG |
| TCGA- | 278 | GAGATCATGTCCTAACCCTGATCCTCTTGTCCCACAGATATCCAGAACCCTCTAAGAAGTTCCTGCTatataaTCGAttaattaatattaatttaaatatttatttGAATGTGGTTAGAGACACGTGTT |
| 176 | | TGACCCTGCCGTGTACCAGCTGAGAGACTCTAAATCCAGTGACAAGTCTG |
| TCGA- | 279 | GAGATCATGTCCTAACCCTGATCCTCTTGTCCCACAGATATCCAGAACCCTCTAAGAAGTTCCTGCTttatataaTCGAttaattaatattaatttaaatatttatGAATGTGGTTAGAGACACGTTGA |
| 177 | | TGACCCTGCCGTGTACCAGCTGAGAGACTCTAAATCCAGTGACAAGTCTG |
| TCGA- | 280 | GAGATCATGTCCTAACCCTGATCCTCTTGTCCCACAGATATCCAGAACCCTCTAAGAAGTTCCTGCTatttatataaTCGAttaattaatattaatttaaatatttGAATGTGGTTAGAGACACGTTGT |
| 178 | | TGACCCTGCCGTGTACCAGCTGAGAGACTCTAAATCCAGTGACAAGTCTG |
| TCGA- | 281 | GAGATCATGTCCTAACCCTGATCCTCTTGTCCCACAGATATCCAGAACCCTCTAAGAAGTTCCTGCTttatttatataaTCGAttaattaatattaatttaaatatGAATGTGGTTAGAGACACGTTTC |
| 179 | | TGACCCTGCCGTGTACCAGCTGAGAGACTCTAAATCCAGTGACAAGTCTG |
| TCGA- | 282 | GAGATCATGTCCTAACCCTGATCCTCTTGTCCCACAGATATCCAGAACCCTCTAAGAAGTTCCTGCTatttatttatataaTCGAttaattaatattaatttaaatGAATGTGGTTAGAGACACTAACC |
| 180 | | GTGACCCTCCGTGTACCAGCTGAGAGACTCTAAATCCAGTGACAAGTCTG |
| TCGA- | 283 | GAGATCATGTCCTAACCCTGATCCTCTTGTCCCACAGATATCCAGAACCCTCTAAGAAGTTCCTGCTatatttatttatataaTCGAttaattaatattaatttaaGAATGTGGTTAGAGACACTAACG |
| 181 | | TGACCCTGCCGTGTACCAGCTGAGAGACTCTAAATCCAGTGACAAGTCTG |
| TCGA- | 284 | GAGATCATGTCCTAACCCTGATCCTCTTGTCCCACAGATATCCAGAACCCTCTAAGAAGTTCCTGCTaaatatttatttatataaTCGAttaattaatattaatttGAATGTGGTTAGAGACACTACT |
| 182 | | GTGACCCTGCCGTGTACCAGCTGAGAGACTCTAAATCCAGTGACAAGTCTG |
| TCGA- | 285 | GAGATCATGTCCTAACCCTGATCCTCTTGTCCCACAGATATCCAGAACCCTCTAAGAAGTTCCTGCTttaaatatttatttatataaTCGAttaattaatattaatGAATGTGGTTAGAGACACTAGAG |
| 183 | | TGACCCTGCCGTGTACCAGCTGAGAGACTCTAAATCCAGTGACAAGTCTG |
| TCGA- | 286 | GAGATCATGTCCTAACCCTGATCCTCTTGTCCCACAGATATCCAGAACCCTCTAAGAAGTTCCTGCTatttaaatatttatttatataaTCGAttaattaatattaGAATGTGGTTAGAGACACTAGC |
| 184 | | ATGACCCTGCCGTGTACCAGCTGAGAGACTCTAAATCCAGTGACAAGTCTG |
| TCGA- | 287 | GAGATCATGTCCTAACCCTGATCCTCTTGTCCCACAGATATCCAGAACCCTCTAAGAAGTTCCTGCTtaatttaaatatttatttatataaTCGAttaattaatatGAATGTGGTTAGAGACACTAG |
| 185 | | TTTGACCCTGCCGTGTACCAGCTGAGAGACTCTAAATCCAGTGACAAGTCTG |
| TCGA- | 288 | GAGATCATGTCCTAACCCTGATCCTCTTGTCCCACAGATATCCAGAACCCTCTAAGAAGTTCCTGCTattaatttaaatatttatttatataaTCGAttaattaatGAATGTGGTTAGAGACACTATC |
| 186 | | CTGACCCTGCCGTGTACCAGCTGAGAGACTCTAAATCCAGTGACAAGTCTG |
| TCGA- | 289 | GAGATCATGTCCTAACCCTGATCCTCTTGTCCCACAGATATCCAGAACCCTCTAAGAAGTTCCTGCTatattaatttaaatatttatttatataaTCGAttaattaGAATGTGGTTAGAGACACTATG |
| 187 | | ATGACCCTGCCGTGTACCAGCTGAGAGACTCTAAATCCAGTGACAAGTCTG |
| TCGA- | 290 | GAGATCATGTCCTAACCCTGATCCTCTTGTCCCACAGATATCCAGAACCCTCTAAGAAGTTCCTGCTtaatattaatttaaatatttatttatataaTCGAttaatGAATGTGGTTAGAGACACTCAAA |
| 188 | | TGACCCTGCCGTGTACCAGCTGAGAGACTCTAAATCCAGTGACAAGTCTG |
| TCGA- | 291 | GAGATCATGTCCTAACCCTGATCCTCTTGTCCCACAGATATCCAGAACCCTCTAAGAAGTTCCTGCTattaatattaatttaaatatttatttatataaTCGAttaGAATGTGGTTAGAGACACTCAA |
| 189 | | GTGACCCTGCCGTGTACCAGCTGAGAGACTCTAAATCCAGTGACAAGTCTG |
| TCGA- | 292 | GAGATCATGTCCTAACCCTGATCCTCTTGTCCCACAGATATCCAGAACCCTCTAAGAAGTTCCTGCTtaattaatattaatttaaatatttatttatataaTCGAtGAATGTGGTTAGAGACACTCTAA |
| 190 | | GTGACCCTCCGTGTACCAGCTGAGAGACTCTAAATCCAGTGACAAGTCTG |
| TCGA- | 293 | GAGATCATGTCCTAACCCTGATCCTCTTGTCCCACAGATATCCAGAACCCTCTAAGAAGTTCCTGCTAttaattaatattaatttaaatatttatttatataaTCGGAATGTGGTTAGAGACACTCTAG |
| 191 | | TGACCCTGCCGTGTACCAGCTGAGAGACTCTAAATCCAGTGACAAGTCTG |

TABLE 4
Sequences of individual ssODN used to assess the TC context in TALE base editors target
sequences in Example 4

| SEQ | | |
|---|---|---|
| ID | | |
| NO# | Name | Target polynucleotide sequences |
| 294 | TC_1 | GAGATCATGTCCTAACCCTGATCCTCTTGTCCCACAGATATCCAGAACCCTCTAAGAAGTTCCTGCTTTAATATTATATCCAAGAATGT |
| | | GGTTAGAGACATGACCCTGCCGTGTACCAGCTGAGAGACTCTAAATCCAGTGACAAGTCTG |
| 295 | TC_2 | GAGATCATGTCCTAACCCTGATCCTCTTGTCCCACAGATATCCAGAACCCTCTAAGAAGTTCCTGCTATTATAATATATCCATGAATGT |
| | | GGTTAGAGACATGACCCTGCCGTGTACCAGCTGAGAGACTCTAAATCCAGTGACAAGTCTG |
| 296 | TC_3 | GAGATCATGTCCTAACCCTGATCCTCTTGTCCCACAGATATCCAGAACCCTCTAAGAAGTTCCTGCTAATAATAATAATCCACGAATGT |
| | | GGTTAGAGACATGACCCTGCCGTGTACCAGCTGAGAGACTCTAAATCCAGTGACAAGTCTG |
| 297 | TC_4 | GAGATCATGTCCTAACCCTGATCCTCTTGTCCCACAGATATCCAGAACCCTCTAAGAAGTTCCTGCTTAAATATATTATCCAGGAATGT |
| | | GGTTAGAGACATGACCCTGCCGTGTACCAGCTGAGAGACTCTAAATCCAGTGACAAGTCTG |
| 298 | TC_5 | GAGATCATGTCCTAACCCTGATCCTCTTGTCCCACAGATATCCAGAACCCTCTAAGAAGTTCCTGCTTTAAATTATAATCCTAGAATGT |
| | | GGTTAGAGACATGACCCTGCCGTGTACCAGCTGAGAGACTCTAAATCCAGTGACAAGTCTG |
| 299 | TC_6 | GAGATCATGTCCTAACCCTGATCCTCTTGTCCCACAGATATCCAGAACCCTCTAAGAAGTTCCTGCTAAAATTATTTATCCTTGAATGT |
| | | GGTTAGAGACATGACCCTGCCGTGTACCAGCTGAGAGACTCTAAATCCAGTGACAAGTCTG |
| 300 | TC_7 | GAGATCATGTCCTAACCCTGATCCTCTTGTCCCACAGATATCCAGAACCCTCTAAGAAGTTCCTGCTTATTATTTTAATCCTCGAATGT |
| | | GGTTAGAGACATGACCCTGCCGTGTACCAGCTGAGAGACTCTAAATCCAGTGACAAGTCTG |
| 301 | TC_8 | GAGATCATGTCCTAACCCTGATCCTCTTGTCCCACAGATATCCAGAACCCTCTAAGAAGTTCCTGCTTTTATAATTAATCCTGGAATGT |
| | | GGTTAGAGACATGACCCTGCCGTGTACCAGCTGAGAGACTCTAAATCCAGTGACAAGTCTG |
| 302 | TC_9 | GAGATCATGTCCTAACCCTGATCCTCTTGTCCCACAGATATCCAGAACCCTCTAAGAAGTTCCTGCTAATATATTATATCCCAGAATGT |
| | | GGTTAGAGACATGACCCTGCCGTGTACCAGCTGAGAGACTCTAAATCCAGTGACAAGTCTG |
| 303 | TC_10 | GAGATCATGTCCTAACCCTGATCCTCTTGTCCCACAGATATCCAGAACCCTCTAAGAAGTTCCTGCTTTAAATATATATCCCTGAATGT |
| | | GGTTAGAGACATGACCCTGCCGTGTACCAGCTGAGAGACTCTAAATCCAGTGACAAGTCTG |
| 304 | TC_11 | GAGATCATGTCCTAACCCTGATCCTCTTGTCCCACAGATATCCAGAACCCTCTAAGAAGTTCCTGCTTTAATAAATAATCCCCGAATGT |
| | | GGTTAGAGACATGACCCTGCCGTGTACCAGCTGAGAGACTCTAAATCCAGTGACAAGTCTG |
| 305 | TC_12 | GAGATCATGTCCTAACCCTGATCCTCTTGTCCCACAGATATCCAGAACCCTCTAAGAAGTTCCTGCTTAAAATTTAAATCCCGGAATGT |
| | | GGTTAGAGACATGACCCTGCCGTGTACCAGCTGAGAGACTCTAAATCCAGTGACAAGTCTG |
| 306 | TC_13 | GAGATCATGTCCTAACCCTGATCCTCTTGTCCCACAGATATCCAGAACCCTCTAAGAAGTTCCTGCTTATTAAATATATCCGAGAATGT |
| | | GGTTAGAGACATGACCCTGCCGTGTACCAGCTGAGAGACTCTAAATCCAGTGACAAGTCTG |
| 307 | TC_14 | GAGATCATGTCCTAACCCTGATCCTCTTGTCCCACAGATATCCAGAACCCTCTAAGAAGTTCCTGCTAATATAAATTATCCGTGAATGT |
| | | GGTTAGAGACATGACCCTGCCGTGTACCAGCTGAGAGACTCTAAATCCAGTGACAAGTCTG |
| 308 | TC_15 | GAGATCATGTCCTAACCCTGATCCTCTTGTCCCACAGATATCCAGAACCCTCTAAGAAGTTCCTGCTTAAAATAAAAATCCGCGAATGT |
| | | GGTTAGAGACATGACCCTGCCGTGTACCAGCTGAGAGACTCTAAATCCAGTGACAAGTCTG |
| 309 | TC_16 | GAGATCATGTCCTAACCCTGATCCTCTTGTCCCACAGATATCCAGAACCCTCTAAGAAGTTCCTGCTATAAAATTATATCCGGGAATGT |
| | | GGTTAGAGACATGACCCTGCCGTGTACCAGCTGAGAGACTCTAAATCCAGTGACAAGTCTG |
| 310 | TC_17 | GAGATCATGTCCTAACCCTGATCCTCTTGTCCCACAGATATCCAGAACCCTCTAAGAAGTTCCTGCTAAATTTTTTTTTCCAAGAATGT |
| | | GGTTAGAGACATGACCCTGCCGTGTACCAGCTGAGAGACTCTAAATCCAGTGACAAGTCTG |
| 311 | TC_18 | GAGATCATGTCCTAACCCTGATCCTCTTGTCCCACAGATATCCAGAACCCTCTAAGAAGTTCCTGCTATATTATATATTCCATGAATGT |
| | | GGTTAGAGACATGACCCTGCCGTGTACCAGCTGAGAGACTCTAAATCCAGTGACAAGTCTG |
| 312 | TC_19 | GAGATCATGTCCTAACCCTGATCCTCTTGTCCCACAGATATCCAGAACCCTCTAAGAAGTTCCTGCTTATTTAATTATTCCACGAATGT |
| | | GGTTAGAGACATGACCCTGCCGTGTACCAGCTGAGAGACTCTAAATCCAGTGACAAGTCTG |
| 313 | TC_20 | GAGATCATGTCCTAACCCTGATCCTCTTGTCCCACAGATATCCAGAACCCTCTAAGAAGTTCCTGCTTTTATTTTAATTCCAGGAATGT |
| | | GGTTAGAGACATGACCCTGCCGTGTACCAGCTGAGAGACTCTAAATCCAGTGACAAGTCTG |
| 314 | TC_21 | GAGATCATGTCCTAACCCTGATCCTCTTGTCCCACAGATATCCAGAACCCTCTAAGAAGTTCCTGCTTAATATTATATTCCTAGAATGT |
| | | GGTTAGAGACATGACCCTGCCGTGTACCAGCTGAGAGACTCTAAATCCAGTGACAAGTCTG |
| 315 | TC_22 | GAGATCATGTCCTAACCCTGATCCTCTTGTCCCACAGATATCCAGAACCCTCTAAGAAGTTCCTGCTATAATAAAATTTCCTTGAATGT |
| | | GGTTAGAGACATGACCCTGCCGTGTACCAGCTGAGAGACTCTAAATCCAGTGACAAGTCTG |
| 316 | TC_23 | GAGATCATGTCCTAACCCTGATCCTCTTGTCCCACAGATATCCAGAACCCTCTAAGAAGTTCCTGCTTTTTATAAAATTCCTCGAATGT |
| | | GGTTAGAGACATGACCCTGCCGTGTACCAGCTGAGAGACTCTAAATCCAGTGACAAGTCTG |
| 317 | TC_24 | GAGATCATGTCCTAACCCTGATCCTCTTGTCCCACAGATATCCAGAACCCTCTAAGAAGTTCCTGCTTTATATATAATTCCTGGAATGT |
| | | GGTTAGAGACATGACCCTGCCGTGTACCAGCTGAGAGACTCTAAATCCAGTGACAAGTCTG |
| 318 | TC_25 | GAGATCATGTCCTAACCCTGATCCTCTTGTCCCACAGATATCCAGAACCCTCTAAGAAGTTCCTGCTAATAATTTTATTCCCAGAATGT |
| | | GGTTAGAGACATGACCCTGCCGTGTACCAGCTGAGAGACTCTAAATCCAGTGACAAGTCTG |
| 319 | TC_26 | GAGATCATGTCCTAACCCTGATCCTCTTGTCCCACAGATATCCAGAACCCTCTAAGAAGTTCCTGCTTTTTAATTAATTCCCTGAATGT |
| | | GGTTAGAGACATGACCCTGCCGTGTACCAGCTGAGAGACTCTAAATCCAGTGACAAGTCTG |
| 320 | TC_27 | GAGATCATGTCCTAACCCTGATCCTCTTGTCCCACAGATATCCAGAACCCTCTAAGAAGTTCCTGCTATATTAATATTTCCCCGAATGT |
| | | GGTTAGAGACATGACCCTGCCGTGTACCAGCTGAGAGACTCTAAATCCAGTGACAAGTCTG |
| 321 | TC_28 | GAGATCATGTCCTAACCCTGATCCTCTTGTCCCACAGATATCCAGAACCCTCTAAGAAGTTCCTGCTTAATATATATTTCCCGGAATGT |
| | | GGTTAGAGACATGACCCTGCCGTGTACCAGCTGAGAGACTCTAAATCCAGTGACAAGTCTG |
| 322 | TC_29 | GAGATCATGTCCTAACCCTGATCCTCTTGTCCCACAGATATCCAGAACCCTCTAAGAAGTTCCTGCTAATAAAATATTTCCGAGAATGT |
| | | GGTTAGAGACATGACCCTGCCGTGTACCAGCTGAGAGACTCTAAATCCAGTGACAAGTCTG |
| 323 | TC_30 | GAGATCATGTCCTAACCCTGATCCTCTTGTCCCACAGATATCCAGAACCCTCTAAGAAGTTCCTGCTAATTTATAATTTCCGTGAATGT |
| | | GGTTAGAGACATGACCCTGCCGTGTACCAGCTGAGAGACTCTAAATCCAGTGACAAGTCTG |
| 324 | TC_31 | GAGATCATGTCCTAACCCTGATCCTCTTGTCCCACAGATATCCAGAACCCTCTAAGAAGTTCCTGCTTAAAATTAATTTCCGCGAATGT |
| | | GGTTAGAGACATGACCCTGCCGTGTACCAGCTGAGAGACTCTAAATCCAGTGACAAGTCTG |
| 325 | TC_32 | GAGATCATGTCCTAACCCTGATCCTCTTGTCCCACAGATATCCAGAACCCTCTAAGAAGTTCCTGCTAAATTATTAATTCCGGGAATGT |
| | | GGTTAGAGACATGACCCTGCCGTGTACCAGCTGAGAGACTCTAAATCCAGTGACAAGTCTG |
| 326 | TC_33 | GAGATCATGTCCTAACCCTGATCCTCTTGTCCCACAGATATCCAGAACCCTCTAAGAAGTTCCTGCTATTTTAAAAACTCCAAGAATGT |
| | | GGTTAGAGACATGACCCTGCCGTGTACCAGCTGAGAGACTCTAAATCCAGTGACAAGTCTG |
| 327 | TC_34 | GAGATCATGTCCTAACCCTGATCCTCTTGTCCCACAGATATCCAGAACCCTCTAAGAAGTTCCTGCTTTTAAATATTCTCCATGAATGT |
| | | GGTTAGAGACATGACCCTGCCGTGTACCAGCTGAGAGACTCTAAATCCAGTGACAAGTCTG |
| 328 | TC_35 | GAGATCATGTCCTAACCCTGATCCTCTTGTCCCACAGATATCCAGAACCCTCTAAGAAGTTCCTGCTATTATTATAACTCCACGAATGT |
| | | GGTTAGAGACATGACCCTGCCGTGTACCAGCTGAGAGACTCTAAATCCAGTGACAAGTCTG |
| 329 | TC_36 | GAGATCATGTCCTAACCCTGATCCTCTTGTCCCACAGATATCCAGAACCCTCTAAGAAGTTCCTGCTTAATTTAAATCTCCAGGAATGT |
| | | GGTTAGAGACATGACCCTGCCGTGTACCAGCTGAGAGACTCTAAATCCAGTGACAAGTCTG |
| 330 | TC_37 | GAGATCATGTCCTAACCCTGATCCTCTTGTCCCACAGATATCCAGAACCCTCTAAGAAGTTCCTGCTTATATAATATCTCCTAGAATGT |
| | | GGTTAGAGACATGACCCTGCCGTGTACCAGCTGAGAGACTCTAAATCCAGTGACAAGTCTG |
| 331 | TC_38 | GAGATCATGTCCTAACCCTGATCCTCTTGTCCCACAGATATCCAGAACCCTCTAAGAAGTTCCTGCTATTAAATAATCTCCTTGAATGT |
| | | GGTTAGAGACATGACCCTGCCGTGTACCAGCTGAGAGACTCTAAATCCAGTGACAAGTCTG |
| 332 | TC_39 | GAGATCATGTCCTAACCCTGATCCTCTTGTCCCACAGATATCCAGAACCCTCTAAGAAGTTCCTGCTATTAAATATACTCCTCGAATGT |
| | | GGTTAGAGACATGACCCTGCCGTGTACCAGCTGAGAGACTCTAAATCCAGTGACAAGTCTG |
| 333 | TC_40 | GAGATCATGTCCTAACCCTGATCCTCTTGTCCCACAGATATCCAGAACCCTCTAAGAAGTTCCTGCTAAATAATTATCTCCTGGAATGT |
| | | GGTTAGAGACATGACCCTGCCGTGTACCAGCTGAGAGACTCTAAATCCAGTGACAAGTCTG |
| 334 | TC_41 | GAGATCATGTCCTAACCCTGATCCTCTTGTCCCACAGATATCCAGAACCCTCTAAGAAGTTCCTGCTTAATTTTTTACTCCCAGAATGT |
| | | GGTTAGAGACATGACCCTGCCGTGTACCAGCTGAGAGACTCTAAATCCAGTGACAAGTCTG |
| 335 | TC_42 | GAGATCATGTCCTAACCCTGATCCTCTTGTCCCACAGATATCCAGAACCCTCTAAGAAGTTCCTGCTTTATATTAAACTCCCTGAATGT |
| | | GGTTAGAGACATGACCCTGCCGTGTACCAGCTGAGAGACTCTAAATCCAGTGACAAGTCTG |
| 336 | TC_43 | GAGATCATGTCCTAACCCTGATCCTCTTGTCCCACAGATATCCAGAACCCTCTAAGAAGTTCCTGCTTTAATTAATTCTCCCCGAATGT |
| | | GGTTAGAGACATGACCCTGCCGTGTACCAGCTGAGAGACTCTAAATCCAGTGACAAGTCTG |
| 337 | TC_44 | GAGATCATGTCCTAACCCTGATCCTCTTGTCCCACAGATATCCAGAACCCTCTAAGAAGTTCCTGCTAATTATTAAACTCCCGGAATGT |
| | | GGTTAGAGACATGACCCTGCCGTGTACCAGCTGAGAGACTCTAAATCCAGTGACAAGTCTG |
| 338 | TC_45 | GAGATCATGTCCTAACCCTGATCCTCTTGTCCCACAGATATCCAGAACCCTCTAAGAAGTTCCTGCTTTATTTATTACTCCGAGAATGT |
| | | GGTTAGAGACATGACCCTGCCGTGTACCAGCTGAGAGACTCTAAATCCAGTGACAAGTCTG |
| 339 | TC_46 | GAGATCATGTCCTAACCCTGATCCTCTTGTCCCACAGATATCCAGAACCCTCTAAGAAGTTCCTGCTTTTTTATATACTCCGTGAATGT |
| | | GGTTAGAGACATGACCCTGCCGTGTACCAGCTGAGAGACTCTAAATCCAGTGACAAGTCTG |
| 340 | TC_47 | GAGATCATGTCCTAACCCTGATCCTCTTGTCCCACAGATATCCAGAACCCTCTAAGAAGTTCCTGCTTTTAAATTATCTCCGCGAATGT |
| | | GGTTAGAGACATGACCCTGCCGTGTACCAGCTGAGAGACTCTAAATCCAGTGACAAGTCTG |
| 341 | TC_48 | GAGATCATGTCCTAACCCTGATCCTCTTGTCCCACAGATATCCAGAACCCTCTAAGAAGTTCCTGCTTTTATATAATCTCCGGGAATGT |
| | | GGTTAGAGACATGACCCTGCCGTGTACCAGCTGAGAGACTCTAAATCCAGTGACAAGTCTG |
| 342 | TC_49 | GAGATCATGTCCTAACCCTGATCCTCTTGTCCCACAGATATCCAGAACCCTCTAAGAAGTTCCTGCTAAAATATTTTGTCCAAGAATGT |
| | | GGTTAGAGACATGACCCTGCCGTGTACCAGCTGAGAGACTCTAAATCCAGTGACAAGTCTG |
| 343 | TC_50 | GAGATCATGTCCTAACCCTGATCCTCTTGTCCCACAGATATCCAGAACCCTCTAAGAAGTTCCTGCTATTTAAATTAGTCCATGAATGT |
| | | GGTTAGAGACATGACCCTGCCGTGTACCAGCTGAGAGACTCTAAATCCAGTGACAAGTCTG |
| 344 | TC_51 | GAGATCATGTCCTAACCCTGATCCTCTTGTCCCACAGATATCCAGAACCCTCTAAGAAGTTCCTGCTATTAATATTTGTCCACGAATGT |
| | | GGTTAGAGACATGACCCTGCCGTGTACCAGCTGAGAGACTCTAAATCCAGTGACAAGTCTG |
| 345 | TC_52 | GAGATCATGTCCTAACCCTGATCCTCTTGTCCCACAGATATCCAGAACCCTCTAAGAAGTTCCTGCTATAAATTTTAGTCCAGGAATGT |
| | | GGTTAGAGACATGACCCTGCCGTGTACCAGCTGAGAGACTCTAAATCCAGTGACAAGTCTG |
| 346 | TC_53 | GAGATCATGTCCTAACCCTGATCCTCTTGTCCCACAGATATCCAGAACCCTCTAAGAAGTTCCTGCTATAAAATAAAGTCCTAGAATGT |
| | | GGTTAGAGACATGACCCTGCCGTGTACCAGCTGAGAGACTCTAAATCCAGTGACAAGTCTG |
| 347 | TC_54 | GAGATCATGTCCTAACCCTGATCCTCTTGTCCCACAGATATCCAGAACCCTCTAAGAAGTTCCTGCTTATATTTTATGTCCTTGAATGT |
| | | GGTTAGAGACATGACCCTGCCGTGTACCAGCTGAGAGACTCTAAATCCAGTGACAAGTCTG |
| 348 | TC_55 | GAGATCATGTCCTAACCCTGATCCTCTTGTCCCACAGATATCCAGAACCCTCTAAGAAGTTCCTGCTATAATTATATGTCCTCGAATGT |
| | | GGTTAGAGACATGACCCTGCCGTGTACCAGCTGAGAGACTCTAAATCCAGTGACAAGTCTG |
| 349 | TC_56 | GAGATCATGTCCTAACCCTGATCCTCTTGTCCCACAGATATCCAGAACCCTCTAAGAAGTTCCTGCTATTTAATATTGTCCTGGAATGT |
| | | GGTTAGAGACATGACCCTGCCGTGTACCAGCTGAGAGACTCTAAATCCAGTGACAAGTCTG |
| 350 | TC_57 | GAGATCATGTCCTAACCCTGATCCTCTTGTCCCACAGATATCCAGAACCCTCTAAGAAGTTCCTGCTTTTTTATTATGTCCCAGAATGT |
| | | GGTTAGAGACATGACCCTGCCGTGTACCAGCTGAGAGACTCTAAATCCAGTGACAAGTCTG |
| 351 | TC_58 | GAGATCATGTCCTAACCCTGATCCTCTTGTCCCACAGATATCCAGAACCCTCTAAGAAGTTCCTGCTTTAAATTTTTGTCCCTGAATGT |
| | | GGTTAGAGACATGACCCTGCCGTGTACCAGCTGAGAGACTCTAAATCCAGTGACAAGTCTG |
| 352 | TC_59 | GAGATCATGTCCTAACCCTGATCCTCTTGTCCCACAGATATCCAGAACCCTCTAAGAAGTTCCTGCTTTTATAAAAAGTCCCCGAATGT |
| | | GGTTAGAGACATGACCCTGCCGTGTACCAGCTGAGAGACTCTAAATCCAGTGACAAGTCTG |
| 353 | TC_60 | GAGATCATGTCCTAACCCTGATCCTCTTGTCCCACAGATATCCAGAACCCTCTAAGAAGTTCCTGCTATTAAATTAAGTCCCGGAATGT |
| | | GGTTAGAGACATGACCCTGCCGTGTACCAGCTGAGAGACTCTAAATCCAGTGACAAGTCTG |
| 354 | TC_61 | GAGATCATGTCCTAACCCTGATCCTCTTGTCCCACAGATATCCAGAACCCTCTAAGAAGTTCCTGCTTTAATAATTTGTCCGAGAATGT |
| | | GGTTAGAGACATGACCCTGCCGTGTACCAGCTGAGAGACTCTAAATCCAGTGACAAGTCTG |
| 355 | TC_62 | GAGATCATGTCCTAACCCTGATCCTCTTGTCCCACAGATATCCAGAACCCTCTAAGAAGTTCCTGCTATTTTTATTTGTCCGTGAATGT |
| | | GGTTAGAGACATGACCCTGCCGTGTACCAGCTGAGAGACTCTAAATCCAGTGACAAGTCTG |
| 356 | TC_63 | GAGATCATGTCCTAACCCTGATCCTCTTGTCCCACAGATATCCAGAACCCTCTAAGAAGTTCCTGCTAAAATAATTAGTCCGCGAATGT |
| | | GGTTAGAGACATGACCCTGCCGTGTACCAGCTGAGAGACTCTAAATCCAGTGACAAGTCTG |
| 357 | TC_64 | GAGATCATGTCCTAACCCTGATCCTCTTGTCCCACAGATATCCAGAACCCTCTAAGAAGTTCCTGCTTTATTAATAAGTCCGGGAATGT |
| | | GGTTAGAGACATGACCCTGCCGTGTACCAGCTGAGAGACTCTAAATCCAGTGACAAGTCTG |

### Example 4: Application to the TALE Base Editor Rules to Generate CD52 Negative T Cells

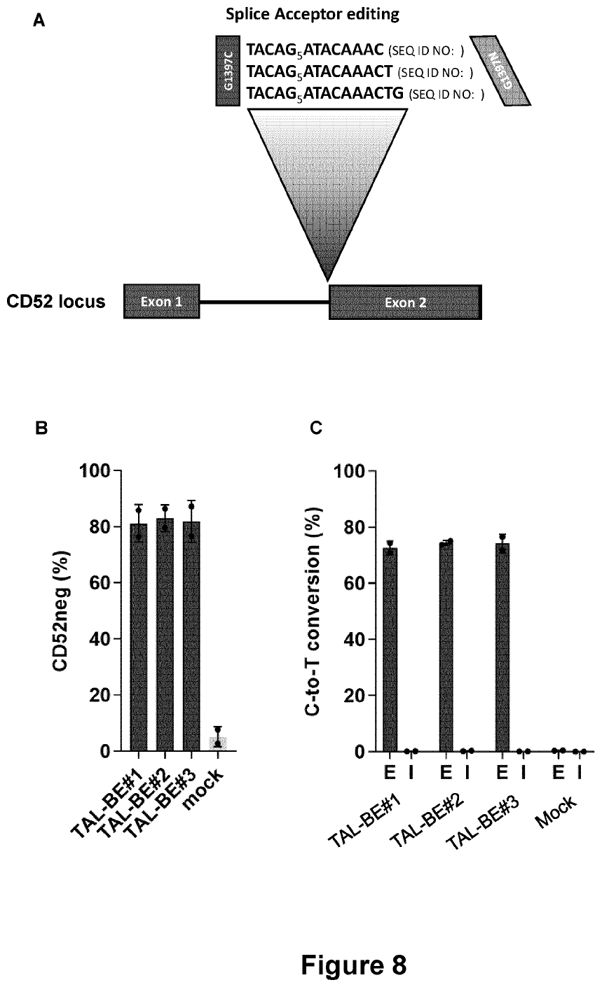

In the context of allogeneic CAR-T therapies, CD52 is often knocked out via gene editing to create resistance to alemtuzumab, a CD52 targeting monoclonal antibody used in lymphodepleting regimens. Because the CD52 gene only has two exons, and the exon 2 contains the sequence coding for the mature peptide, splice site mutation at the intron 1/exon 2 junction was chosen to cause the skipping of exon2, leading to the loss of CD52. The TALE base editors rules defined above were thus applied to identify optimum targets, leading to 3 lead TALE base editors (among 34 potential base editors, FIG. 8A). Primary T cells were transfected with mRNA encoding these three pairs of TALE base editors (TALEB #1 SEQ ID NO: 20 and SEQ ID NO:21; TALEB #2 SEQ ID NO: 22 and SEQ ID NO:23; TALEB #3 SEQ ID NO:24 and SEQ ID NO:25). Seven days post transfection, phenotypic CD52 knock-out was monitored by flow cytometry and splice site editing was measured by NGS. We observed high level of phenotypic knock-out for the three TALE base editors (FIG. 8B, TALEB #1 mean 81.1%+/−4.7%, TALEB #2 SA-2 mean 83%+/−3.4% and TALEB #3 mean 81.9%, +/−5.3%), correlating with editing levels (TALEB #1 mean 72.6%, +/−1.7%, TALEB #2 mean 74.5%, +/−0.6%. and TALEB #3 mean 74.2%, +/−2.3%, FIG. 8C). As expected from our previous datasets, NGS data analysis results showed very low levels of Indels at these sites (TALEB #1 mean 0.16%, +/−0.05%; TALEB #2 mean 0.28%, +/−0.06%; TALEB #3 mean 0.12%, +/−0.02%, Mock transfected mean 0.01%, +/−0.005%; FIG. 8C). Polypeptides and polynucleotide target sequences are reported in Table 5.

TABLE 5
KO CD52 TALEB polypeptides and target polynucleotides
as per the present invention

| SEQ | | |
|---|---|---|
| ID:# | Name | Polynucleotide or polypeptide sequences |
| 358 | CD52 TALE-BE #1 | TTTTGTCCTGAGAGTCCagtttgtatctgtaGGAGGAGAAGTGGGATA |
| | target | |
| 359 | CD52 TALE-BE #2 | TTTGTCCTGAGAGTCCAgtttgtatctgtaGGAGGAGAAGTGGGATA |
| | target | |
| 360 | CD52 TALE-BE #3 | TTGTCCTGAGAGTCCAGtttgtatctgtaGGAGGAGAAGTGGGATA |
| | target | |
| 361 | CD52 TALE-BE | TGGCTGGTGTCGTTTTGtcctgagagtccagtTTGTATCTGTAGGAGGA |
| | SP target | |
| 20 | CD52 TALE-BE | MGDPKKKRKVIDIADLRTLGYSQQQQEKIKPKVRSTVAQHHEALVGHGFTHAHI |
| | #1-L | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPQQVVAIAS |
| | | NGGGKQALETVQRLLPVLCQAHGLTPQQVVAIASNGGGKQALETVQRLLPVLC |
| | | QAHGLTPQQVVAIASNGGGKQALETVQRLLPVLCQAHGLTPQQVVAIASNNG |
| | | GKQALETVQRLLPVLCQAHGLTPQQVVAIASNGGGKQALETVQRLLPVLCQAH |
| | | GLTPEQVVAIASHDGGKQALETVQRLLPVLCQAHGLTPEQVVAIASHDGGKQA |
| | | LETVQRLLPVLCQAHGLTPQQVVAIASNGGGKQALETVQRLLPVLCQAHGLTP |
| | | QQVVAIASNNGGKQALETVQRLLPVLCQAHGLTPEQVVAIASNIGGKQALETV |
| | | QALLPVLCQAHGLTPQQVVAIASNNGGKQALETVQRLLPVLCQAHGLTPEQVV |
| | | AIASNIGGKQALETVQALLPVLCQAHGLTPQQVVAIASNNGGKQALETVQRLLP |
| | | VLCQAHGLTPQQVVAIASNGGGKQALETVQRLLPVLCQAHGLTPEQVVAIASH |
| | | DGGKQALETVQRLLPVLCQAHGLTPQQVVAIASNGGGRPALESIVAQLSRPDP |
| | | ALAALTNDHLVALACLGGRPALDAVKKGLGGSGSYALGPYQISAPQLPAYNGQ |
| | | TVGTFYYVNDAGGLESKVFSSGGPTPYPNYANAGHVEGQSALFMRDNGISEG |
| | | LVFHNNPEGTCGFCVNMTETLLPENAKMTVVPPEGSGGSTNLSDIIEKETGKQ |
| | | LVIQESILMLPEEVEEVIGNKPESDILVHTAYDESTDENVMLLTSDAPEYKPWAL |
| | | VIQDSNGENKIKML |
| 21 | CD52 TALE-BE | MGDPKKKRKVIDIADLRTLGYSQQQQEKIKPKVRSTVAQHHEALVGHGFTHAHI |
| | #1-R | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPEQVVAIASN |
| | | IGGKQALETVQALLPVLCQAHGLTPQQVVAIASNGGGKQALETVQRLLPVLCQ |
| | | AHGLTPEQVVAIASHDGGKQALETVQRLLPVLCQAHGLTPEQVVAIASHDGGK |
| | | QALETVQRLLPVLCQAHGLTPEQVVAIASHDGGKQALETVQRLLPVLCQAHGL |
| | | TPEQVVAIASNIGGKQALETVQALLPVLCQAHGLTPEQVVAIASHDGGKQALET |
| | | VQRLLPVLCQAHGLTPQQVVAIASNGGGKQALETVQRLLPVLCQAHGLTPQQV |
| | | VAIASNGGGKQALETVQRLLPVLCQAHGLTPEQVVAIASHDGGKQALETVQRL |
| | | LPVLCQAHGLTPQQVVAIASNGGGKQALETVQRLLPVLCQAHGLTPEQVVAIA |
| | | SHDGGKQALETVQRLLPVLCQAHGLTPEQVVAIASHDGGKQALETVQRLLPVL |
| | | CQAHGLTPQQVVAIASNGGGKQALETVQRLLPVLCQAHGLTPEQVVAIASHDG |
| | | GKQALETVQRLLPVLCQAHGLTPQQVVAIASNGGGRPALESIVAQLSRPDPAL |
| | | AALTNDHLVALACLGGRPALDAVKKGLGGSAIPVKRGATGETKVFTGNSNSPK |
| | | SPTKGGCSGGSTNLSDIIEKETGKQLVIQESILMLPEEVEEVIGNKPESDILVHTA |
| | | YDESTDENVMLLTSDAPEYKPWALVIQDSNGENKIKML |
| 22 | CD52 TALE-BE | MGDPKKKRKVIDIADLRTLGYSQQQQEKIKPKVRSTVAQHHEALVGHGFTHAHI |
| | #2-L | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPQQVVAIAS |
| | | NGGGKQALETVQRLLPVLCQAHGLTPQQVVAIASNGGGKQALETVQRLLPVLC |
| | | QAHGLTPQQVVAIASNNGGKQALETVQRLLPVLCQAHGLTPQQVVAIASNGG |
| | | GKQALETVQRLLPVLCQAHGLTPEQVVAIASHDGGKQALETVQRLLPVLCQAH |
| | | GLTPEQVVAIASHDGGKQALETVQRLLPVLCQAHGLTPQQVVAIASNGGGKQA |
| | | LETVQRLLPVLCQAHGLTPQQVVAIASNNGGKQALETVQRLLPVLCQAHGLTP |
| | | EQVVAIASNIGGKQALETVQALLPVLCQAHGLTPQQVVAIASNNGGKQALETVQ |
| | | RLLPVLCQAHGLTPEQVVAIASNIGGKQALETVQALLPVLCQAHGLTPQQVVAI |
| | | ASNNGGKQALETVQRLLPVLCQAHGLTPQQVVAIASNGGGKQALETVQRLLPV |
| | | LCQAHGLTPEQVVAIASHDGGKQALETVQRLLPVLCQAHGLTPEQVVAIASHD |
| | | GGKQALETVQRLLPVLCQAHGLTPQQVVAIASNGGGRPALESIVAQLSRPDPA |
| | | LAALTNDHLVALACLGGRPALDAVKKGLGGSGSYALGPYQISAPQLPAYNGQT |
| | | VGTFYYVNDAGGLESKVFSSGGPTPYPNYANAGHVEGQSALFMRDNGISEGL |
| | | VFHNNPEGTCGFCVNMTETLLPENAKMTVVPPEGSGGSTNLSDIIEKETGKQL |
| | | VIQESILMLPEEVEEVIGNKPESDILVHTAYDESTDENVMLLTSDAPEYKPWALV |
| | | IQDSNGENKIKML |
| 23 | CD52 TALE-BE | MGDPKKKRKVIDIADLRTLGYSQQQQEKIKPKVRSTVAQHHEALVGHGFTHAHI |
| | #2-R | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPEQVVAIASN |
| | | IGGKQALETVQALLPVLCQAHGLTPQQVVAIASNGGGKQALETVQRLLPVLCQ |
| | | AHGLTPEQVVAIASHDGGKQALETVQRLLPVLCQAHGLTPEQVVAIASHDGGK |
| | | QALETVQRLLPVLCQAHGLTPEQVVAIASHDGGKQALETVQRLLPVLCQAHGL |
| | | TPEQVVAIASNIGGKQALETVQALLPVLCQAHGLTPEQVVAIASHDGGKQALET |
| | | VQRLLPVLCQAHGLTPQQVVAIASNGGGKQALETVQRLLPVLCQAHGLTPQQV |
| | | VAIASNGGGKQALETVQRLLPVLCQAHGLTPEQVVAIASHDGGKQALETVQRL |
| | | LPVLCQAHGLTPQQVVAIASNGGGKQALETVQRLLPVLCQAHGLTPEQVVAIA |
| | | SHDGGKQALETVQRLLPVLCQAHGLTPEQVVAIASHDGGKQALETVQRLLPVL |
| | | CQAHGLTPQQVVAIASNGGGKQALETVQRLLPVLCQAHGLTPEQVVAIASHDG |
| | | GKQALETVQRLLPVLCQAHGLTPQQVVAIASNGGGRPALESIVAQLSRPDPAL |
| | | AALTNDHLVALACLGGRPALDAVKKGLGGSAIPVKRGATGETKVFTGNSNSPK |
| | | SPTKGGCSGGSTNLSDIIEKETGKQLVIQESILMLPEEVEEVIGNKPESDILVHTA |
| | | YDESTDENVMLLTSDAPEYKPWALVIQDSNGENKIKML |
| 24 | CD52 TALE-BE | MGDPKKKRKVIDIADLRTLGYSQQQQEKIKPKVRSTVAQHHEALVGHGFTHAHI |
| | #3-L | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPQQVVAIAS |
| | | NGGGKQALETVQRLLPVLCQAHGLTPQQVVAIASNNGGKQALETVQRLLPVLC |
| | | QAHGLTPQQVVAIASNGGGKQALETVQRLLPVLCQAHGLTPEQVVAIASHDGG |
| | | KQALETVQRLLPVLCQAHGLTPEQVVAIASHDGGKQALETVQRLLPVLCQAHG |
| | | LTPQQVVAIASNGGGKQALETVQRLLPVLCQAHGLTPQQVVAIASNNGGKQAL |
| | | ETVQRLLPVLCQAHGLTPEQVVAIASNIGGKQALETVQALLPVLCQAHGLTPQQ |
| | | VVAIASNNGGKQALETVQRLLPVLCQAHGLTPEQVVAIASNIGGKQALETVQAL |
| | | LPVLCQAHGLTPQQVVAIASNNGGKQALETVQRLLPVLCQAHGLTPQQVVAIA |
| | | SNGGGKQALETVQRLLPVLCQAHGLTPEQVVAIASHDGGKQALETVQRLLPVL |
| | | CQAHGLTPEQVVAIASHDGGKQALETVQRLLPVLCQAHGLTPEQVVAIASNIG |
| | | GKQALETVQALLPVLCQAHGLTPQQVVAIASNGGGRPALESIVAQLSRPDPAL |
| | | AALTNDHLVALACLGGRPALDAVKKGLGGSGSYALGPYQISAPQLPAYNGQTV |
| | | GTFYYVNDAGGLESKVFSSGGPTPYPNYANAGHVEGQSALFMRDNGISEGLV |
| | | FHNNPEGTCGFCVNMTETLLPENAKMTVVPPEGSGGSTNLSDIIEKETGKQLVI |
| | | QESILMLPEEVEEVIGNKPESDILVHTAYDESTDENVMLLTSDAPEYKPWALVIQ |
| | | DSNGENKIKML |
| 25 | CD52 TALE-BE | MGDPKKKRKVIDIADLRTLGYSQQQQEKIKPKVRSTVAQHHEALVGHGFTHAHI |
| | #3-R | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPEQVVAIASN |
| | | IGGKQALETVQALLPVLCQAHGLTPQQVVAIASNGGGKQALETVQRLLPVLCQ |
| | | AHGLTPEQVVAIASHDGGKQALETVQRLLPVLCQAHGLTPEQVVAIASHDGGK |
| | | QALETVQRLLPVLCQAHGLTPEQVVAIASHDGGKQALETVQRLLPVLCQAHGL |
| | | TPEQVVAIASNIGGKQALETVQALLPVLCQAHGLTPEQVVAIASHDGGKQALET |
| | | VQRLLPVLCQAHGLTPQQVVAIASNGGGKQALETVQRLLPVLCQAHGLTPQQV |
| | | VAIASNGGGKQALETVQRLLPVLCQAHGLTPEQVVAIASHDGGKQALETVQRL |
| | | LPVLCQAHGLTPQQVVAIASNGGGKQALETVQRLLPVLCQAHGLTPEQVVAIA |
| | | SHDGGKQALETVQRLLPVLCQAHGLTPEQVVAIASHDGGKQALETVQRLLPVL |
| | | CQAHGLTPQQVVAIASNGGGKQALETVQRLLPVLCQAHGLTPEQVVAIASHDG |
| | | GKQALETVQRLLPVLCQAHGLTPQQVVAIASNGGGRPALESIVAQLSRPDPAL |
| | | AALTNDHLVALACLGGRPALDAVKKGLGGSAIPVKRGATGETKVFTGNSNSPK |
| | | SPTKGGCSGGSTNLSDIIEKETGKQLVIQESILMLPEEVEEVIGNKPESDILVHTA |
| | | YDESTDENVMLLTSDAPEYKPWALVIQDSNGENKIKML |
| 26 | CD52 TALE-BE | MGDPKKKRKVIDIADLRTLGYSQQQQEKIKPKVRSTVAQHHEALVGHGFTHAHI |
| | SP-L | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPQQVVAIAS |
| | | NNGGKQALETVQRLLPVLCQAHGLTPQQVVAIASNNGGKQALETVQRLLPVLC |
| | | QAHGLTPEQVVAIASHDGGKQALETVQRLLPVLCQAHGLTPQQVVAIASNGGG |
| | | KQALETVQRLLPVLCQAHGLTPQQVVAIASNNGGKQALETVQRLLPVLCQAHG |
| | | LTPQQVVAIASNNGGKQALETVQRLLPVLCQAHGLTPQQVVAIASNGGGKQAL |
| | | ETVQRLLPVLCQAHGLTPQQVVAIASNNGGKQALETVQRLLPVLCQAHGLTPQ |
| | | QVVAIASNGGGKQALETVQRLLPVLCQAHGLTPEQVVAIASHDGGKQALETVQ |
| | | RLLPVLCQAHGLTPQQVVAIASNNGGKQALETVQRLLPVLCQAHGLTPQQVVA |
| | | IASNGGGKQALETVQRLLPVLCQAHGLTPQQVVAIASNGGGKQALETVQRLLP |
| | | VLCQAHGLTPQQVVAIASNGGGKQALETVQRLLPVLCQAHGLTPQQVVAIASN |
| | | GGGKQALETVQRLLPVLCQAHGLTPQQVVAIASNGGGRPALESIVAQLSRPDP |
| | | ALAALTNDHLVALACLGGRPALDAVKKGLGGSGSYALGPYQISAPQLPAYNGQ |
| | | TVGTFYYVNDAGGLESKVFSSGGPTPYPNYANAGHVEGQSALFMRDNGISEG |
| | | LVFHNNPEGTCGFCVNMTETLLPENAKMTVVPPEGSGGSTNLSDIIEKETGKQ |
| | | LVIQESILMLPEEVEEVIGNKPESDILVHTAYDESTDENVMLLTSDAPEYKPWAL |
| | | VIQDSNGENKIKML |
| 27 | CD52 TALE-BE | MGDPKKKRKVIDIADLRTLGYSQQQQEKIKPKVRSTVAQHHEALVGHGFTHAHI |
| | SP-R | VALSQHPAALGTVAVKYQDMIAALPEATHEAIVGVGKQWSGARALEALLTVAG |
| | | ELRGPPLQLDTGQLLKIAKRGGVTAVEAVHAWRNALTGAPLNLTPEQVVAIASH |
| | | DGGKQALETVQRLLPVLCQAHGLTPEQVVAIASHDGGKQALETVQRLLPVLCQ |
| | | AHGLTPQQVVAIASNGGGKQALETVQRLLPVLCQAHGLTPEQVVAIASHDGGK |
| | | QALETVQRLLPVLCQAHGLTPEQVVAIASHDGGKQALETVQRLLPVLCQAHGL |
| | | TPQQVVAIASNGGGKQALETVQRLLPVLCQAHGLTPEQVVAIASNIGGKQALET |
| | | VQALLPVLCQAHGLTPEQVVAIASHDGGKQALETVQRLLPVLCQAHGLTPEQV |
| | | VAIASNIGGKQALETVQALLPVLCQAHGLTPQQVVAIASNNGGKQALETVQRLL |
| | | PVLCQAHGLTPEQVVAIASNIGGKQALETVQALLPVLCQAHGLTPQQVVAIASN |
| | | GGGKQALETVQRLLPVLCQAHGLTPEQVVAIASNIGGKQALETVQALLPVLCQ |
| | | AHGLTPEQVVAIASHDGGKQALETVQRLLPVLCQAHGLTPEQVVAIASNIGGKQ |
| | | ALETVQALLPVLCQAHGLTPQQVVAIASNGGGRPALESIVAQLSRPDPALAALT |
| | | NDHLVALACLGGRPALDAVKKGLGGSAIPVKRGATGETKVFTGNSNSPKSPTK |
| | | GGCSGGSTNLSDIIEKETGKQLVIQESILMLPEEVEEVIGNKPESDILVHTAYDES |
| | | TDENVMLLTSDAPEYKPWALVIQDSNGENKIKML |

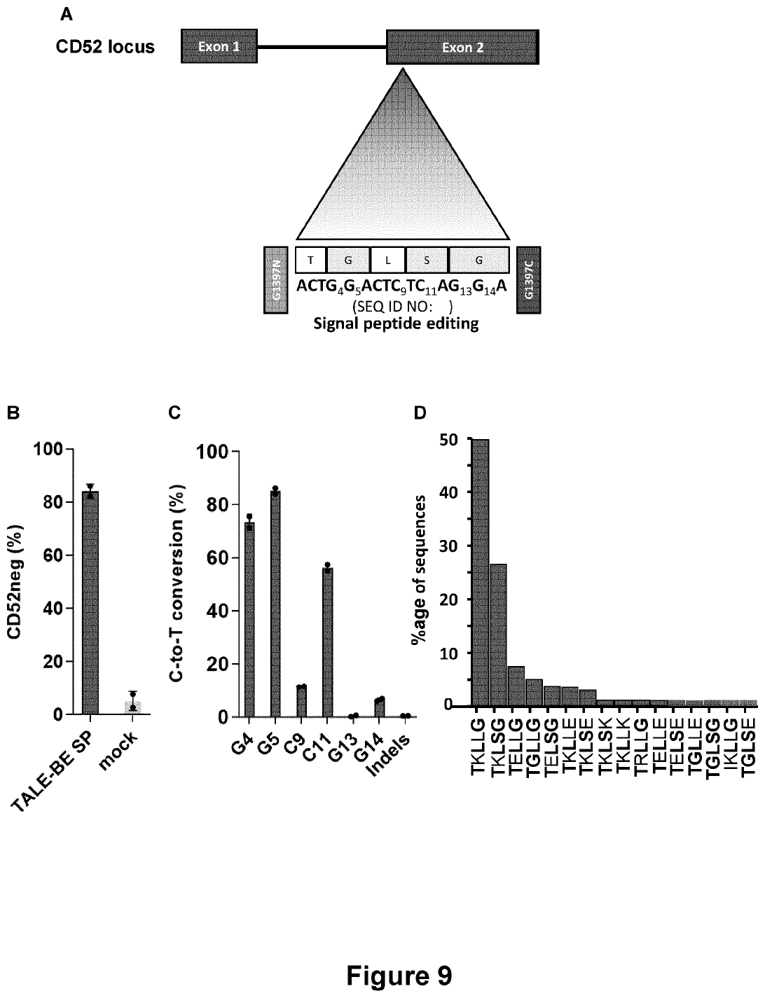

We next sought to use a TALE base editors to create mutations within the CD52 signal peptide sequences (SEQ ID NO:365). Mutations in signal peptide has been shown to disrupt the processing and the translocation of nascent peptides and thus impair the surface expression of certain genes. We thus designed a TALEB: TALE base editor SP (SEQ ID NO:26 and SEQ ID NO:27) that could potentially lead to (i) a silent mutation at Leu23 residue and (ii) several amino acid changes (Gly22Lys, Ser24Leu and Gly25Lys) in the signal peptide (FIG. 9A). Changes in the residues, mutating a hydrophobic glycine to a highly charged lysine and a polar serine to a hydrophobic leucine in the signal peptide, would significantly impact the ability for the signal peptide to correctly direct translocation. Indeed, 6 days post TALE base editor mRNA transfection (ex2 SP), CD52 negative cells were observed by flow cytometry an average of 84.2% (+/−1.8%) (FIG. 9B). The NGS sequencing analysis revealed that all 6 positions were mutated, albeit at different levels (mean editing frequencies: G[4]: 73.65+/−1%, G[5]: 85.65+/−0.7%, C[9]: 11.4+/−0.1% C[11]: 56.5+/−0.9%, G[13]: 0.6+/−0.1, G[14]:6.5+/−0.5%) (FIG. 9C). The sequences analysis revealed that 34 different species at the protein level (including the WT) were identified and present in different proportions (FIG. 9D).

Altogether, a very high phenotypic KO (median CD52 negative population: 82.1%) and editing purity (median=99.7 and s.d.=0.6) was obtained with the 4 CD52 TALEBs. To evaluate possible off-target editing of these 4 CD52 TALEBs, an in-silico list of 276 potential off site targets was generated (Table 6) and monitored using a multiplexed amplicon sequencing assay. Target amplicon sequencing of these sites did not demonstrated evidence of editing above the control experiment (N=2, independent T-cells donors).

TABLE 6
Predicted potential off-targeted site for the 4 TALEB targeting CD52
Chomosomal position

| chromomosome | | | off-site | |
|---|---|---|---|---|
| (GRCh38) | target_start | target_end | id | base_editor |
| chr1 | 2846141 | 2846197 | OT001 | ex2 SP |
| chr1 | 13544690 | 13544754 | OT002 | ex2 SA-2 |
| chr1 | 23480510 | 23480576 | OT003 | ex2 SA-2; ex2 SA-1; ex2 SA-3 |
| chr1 | 23480510 | 23480577 | OT004 | ex2 SA-2; ex2 SA-1; ex2 SA-3 |
| chr1 | 23480510 | 23480578 | OT005 | ex2 SA-2; ex2 SA-1; ex2 SA-3 |
| chr1 | 23933641 | 23933702 | OT006 | ex2 SP |
| chr1 | 55612568 | 55612619 | OT007 | ex2 SA-2; ex2 SA-1 |
| chr1 | 55612569 | 55612619 | OT008 | ex2 SA-2; ex2 SA-1 |
| chr1 | 69516500 | 69516571 | OT009 | ex2 SA-2; ex2 SA-1 |
| chr1 | 69516501 | 69516571 | OT010 | ex2 SA-2; ex2 SA-1 |
| chr1 | 71728841 | 71728906 | OT011 | ex2 SA-2; ex2 SA-1 |
| chr1 | 71728842 | 71728906 | OT012 | ex2 SA-2; ex2 SA-1 |
| chr1 | 81238139 | 81238195 | OT013 | ex2 SP |
| chr1 | 96131553 | 96131604 | OT014 | ex2 SA-1 |
| chr1 | 107799822 | 107799878 | OT015 | ex2 SA-3 |
| chr1 | 124797329 | 124797376 | OT016 | ex2 SP |
| chr1 | 124806508 | 124806555 | OT017 | |
| chr1 | 124840177 | 124840224 | OT018 | |
| chr1 | 154260540 | 154260591 | OT019 | ex2 SA-2; ex2 SA-3 |
| chr1 | 154260541 | 154260591 | OT020 | ex2 SA-2; ex2 SA-3 |
| chr1 | 157452002 | 157452057 | OT021 | ex2 SP |
| chr1 | 158435179 | 158435247 | OT022 | ex2 SA-3 |
| chr1 | 160425343 | 160425405 | OT023 | |
| chr1 | 160425344 | 160425405 | OT024 | |
| chr1 | 174247827 | 174247887 | OT025 | ex2 SP |
| chr1 | 226195588 | 226195639 | OT026 | ex2 SA-3 |
| chr1 | 227743091 | 227743161 | OT027 | ex2 SA-2 |
| chr1 | 227931846 | 227931909 | OT028 | ex2 SP |
| chr1 | 243397613 | 243397678 | OT029 | ex2 SP |
| chr2 | 18221619 | 18221672 | OT030 | ex2 SP |
| chr2 | 26845625 | 26845677 | OT031 | ex2 SA-3 |
| chr2 | 32887842 | 32887909 | OT032 | ex2 SA-2; ex2 SA-1; ex2 SA-3 |
| chr2 | 32887843 | 32887909 | OT033 | ex2 SA-2; ex2 SA-1; ex2 SA-3 |
| chr2 | 32887844 | 32887909 | OT034 | ex2 SA-2; ex2 SA-1; ex2 SA-3 |
| chr2 | 47845454 | 47845524 | OT035 | ex2 SA-3 |
| chr2 | 48880763 | 48880836 | OT036 | ex2 SP |
| chr2 | 58437862 | 58437923 | OT037 | ex2 SA-3 |
| chr2 | 67994462 | 67994526 | OT038 | ex2 SA-3 |
| chr2 | 96925576 | 96925648 | OT039 | ex2 SA-2; ex2 SA-1 |
| chr2 | 96925576 | 96925649 | OT040 | ex2 SA-2; ex2 SA-1 |
| chr2 | 98801844 | 98801898 | OT041 | ex2 SP |
| chr2 | 99249799 | 99249863 | OT042 | ex2 SA-3 |
| chr2 | 146918792 | 146918850 | OT043 | ex2 SA-2 |
| chr2 | 158263433 | 158263480 | OT044 | ex2 SA-3 |
| chr2 | 161997174 | 161997225 | OT045 | ex2 SA-2; ex2 SA-1; ex2 SA-3 |
| chr2 | 161997174 | 161997226 | OT046 | ex2 SA-2; ex2 SA-1; ex2 SA-3 |
| chr2 | 161997174 | 161997227 | OT047 | ex2 SA-2; ex2 SA-1; ex2 SA-3 |
| chr2 | 190149889 | 190149962 | OT048 | ex2 SP |
| chr2 | 200590532 | 200590577 | OT049 | ex2 SP |
| chr2 | 201881430 | 201881477 | OT050 | ex2 SA-3 |
| chr2 | 217796288 | 217796355 | OT051 | ex2 SA-3 |
| chr2 | 229305302 | 229305362 | OT052 | ex2 SA-2; ex2 SA-1 |
| chr2 | 229305302 | 229305363 | OT053 | ex2 SA-2; ex2 SA-1 |
| chr2 | 237506320 | 237506371 | OT054 | ex2 SP |
| chr2 | 241547095 | 241547162 | OT055 | ex2 SA-3 |
| chr3 | 14599548 | 14599592 | OT056 | ex2 SA-3 |
| chr3 | 28801521 | 28801575 | OT057 | ex2 SA-2; ex2 SA-1; ex2 SA-3 |
| chr3 | 28801521 | 28801576 | OT058 | ex2 SA-2; ex2 SA-1; ex2 SA-3 |
| chr3 | 28801521 | 28801577 | OT059 | ex2 SA-2; ex2 SA-1; ex2 SA-3 |
| chr3 | 59289276 | 59289345 | OT060 | ex2 SA-2; ex2 SA-3 |
| chr3 | 59289277 | 59289345 | OT061 | ex2 SA-2; ex2 SA-3 |
| chr3 | 62756869 | 62756930 | OT062 | ex2 SA-3 |
| chr3 | 74304300 | 74304349 | OT063 | ex2 SA-2; ex2 SA-1 |
| chr3 | 74304300 | 74304350 | OT064 | ex2 SA-2; ex2 SA-1 |
| chr3 | 91036719 | 91036766 | OT065 | |
| chr3 | 91144616 | 91144663 | OT066 | ex2 SP |
| chr3 | 91170102 | 91170149 | OT067 | ex2 SP |
| chr3 | 109314237 | 109314295 | OT068 | ex2 SA-2; ex2 SA-1 |
| chr3 | 109314237 | 109314296 | OT069 | ex2 SA-2; ex2 SA-1 |
| chr3 | 118544994 | 118545044 | OT070 | ex2 SA-2 |
| chr3 | 146318980 | 146319032 | OT071 | ex2 SP |
| chr3 | 151261360 | 151261413 | OT072 | ex2 SA-3 |
| chr3 | 180318072 | 180318130 | OT073 | ex2 SA-1; ex2 SA-3 |
| chr3 | 180318072 | 180318132 | OT074 | ex2 SA-1; ex2 SA-3 |
| chr3 | 182481327 | 182481389 | OT075 | ex2 SP |
| chr3 | 186932765 | 186932827 | OT076 | ex2 SA-1 |
| chr3 | 188066417 | 188066468 | OT077 | ex2 SA-2; ex2 SA-3 |
| chr3 | 188066418 | 188066468 | OT078 | ex2 SA-2; ex2 SA-3 |
| chr4 | 16815598 | 16815653 | OT079 | ex2 SA-2 |
| chr4 | 17722157 | 17722203 | OT080 | ex2 SA-3 |
| chr4 | 19946560 | 19946628 | OT081 | ex2 SP |
| chr4 | 79828942 | 79828999 | OT082 | ex2 SA-2; ex2 SA-3 |
| chr4 | 79828942 | 79829000 | OT083 | ex2 SA-2; ex2 SA-3 |
| chr4 | 98205286 | 98205358 | OT084 | ex2 SA-1 |
| chr4 | 113839584 | 113839648 | OT085 | ex2 SP |
| chr4 | 155886673 | 155886722 | OT086 | ex2 SA-3 |
| chr4 | 173597379 | 173597431 | OT087 | ex2 SA-1 |
| chr5 | 6220501 | 6220560 | OT088 | ex2 SA-2; ex2 SA-1 |
| chr5 | 6220501 | 6220561 | OT089 | ex2 SA-2; ex2 SA-1 |
| chr5 | 49808606 | 49808652 | OT090 | ex2 SP |
| chr5 | 50031584 | 50031630 | OT091 | ex2 SP |
| chr5 | 91613718 | 91613791 | OT092 | ex2 SA-3 |
| chr5 | 91873149 | 91873193 | OT093 | ex2 SA-2; ex2 SA-1; ex2 SA-3 |
| chr5 | 91873149 | 91873194 | OT094 | ex2 SA-2; ex2 SA-1; ex2 SA-3 |
| chr5 | 91873149 | 91873195 | OT095 | ex2 SA-2; ex2 SA-1; ex2 SA-3 |
| chr5 | 115081887 | 115081960 | OT096 | ex2 SA-2; ex2 SA-3 |
| chr5 | 115081888 | 115081960 | OT097 | ex2 SA-2; ex2 SA-3 |
| chr5 | 125879529 | 125879577 | OT098 | ex2 SA-3 |
| chr5 | 137320608 | 137320667 | OT099 | ex2 SP |
| chr5 | 142341832 | 142341883 | OT100 | ex2 SA-2; ex2 SA-1; ex2 SA-3 |
| chr5 | 142341833 | 142341883 | OT101 | ex2 SA-2; ex2 SA-1; ex2 SA-3 |
| chr5 | 142341834 | 142341883 | OT102 | ex2 SA-2; ex2 SA-1; ex2 SA-3 |
| chr5 | 163022008 | 163022061 | OT103 | ex2 SA-2; ex2 SA-3 |
| chr5 | 163022008 | 163022062 | OT104 | ex2 SA-2; ex2 SA-3 |
| chr6 | 7713258 | 7713317 | OT105 | ex2 SA-1 |
| chr6 | 8426979 | 8427051 | OT106 | ex2 SP |
| chr6 | 32303681 | 32303729 | OT107 | ex2 SA-3 |
| chr6 | 36718354 | 36718426 | OT108 | ex2 SA-2; ex2 SA-1; ex2 SA-3 |
| chr6 | 36718355 | 36718426 | OT109 | ex2 SA-2; ex2 SA-1; ex2 SA-3 |
| chr6 | 36718356 | 36718426 | OT110 | ex2 SA-2; ex2 SA-1; ex2 SA-3 |
| chr6 | 89156592 | 89156640 | OT111 | ex2 SA-3 |
| chr6 | 90255521 | 90255588 | OT112 | ex2 SA-2; ex2 SA-3 |
| chr6 | 90255522 | 90255588 | OT113 | ex2 SA-2; ex2 SA-3 |
| chr6 | 137295812 | 137295863 | OT114 | ex2 SA-2 |
| chr6 | 147181346 | 147181412 | OT115 | ex2 SA-3 |
| chr6 | 154113075 | 154113119 | OT116 | ex2 SA-3 |
| chr6 | 165938016 | 165938074 | OT117 | ex2 SA-2; ex2 SA-1; ex2 SA-3 |
| chr6 | 165938016 | 165938075 | OT118 | ex2 SA-2; ex2 SA-1; ex2 SA-3 |
| chr6 | 165938016 | 165938076 | OT119 | ex2 SA-2; ex2 SA-1; ex2 SA-3 |
| chr7 | 554058 | 554130 | OT120 | ex2 SA-3 |
| chr7 | 22546403 | 22546466 | OT121 | ex2 SP |
| chr7 | 31758951 | 31759014 | OT122 | ex2 SA-1 |
| chr7 | 35835706 | 35835764 | OT123 | ex2 SA-2 |
| chr7 | 39597520 | 39597585 | OT124 | ex2 SA-2 |
| chr7 | 49258425 | 49258472 | OT125 | ex2 SP |
| chr7 | 51917068 | 51917115 | OT126 | ex2 SP |
| chr7 | 64812784 | 64812841 | OT127 | ex2 SA-2; ex2 SA-1 |
| chr7 | 64812785 | 64812841 | OT128 | ex2 SA-2; ex2 SA-1 |
| chr7 | 98655759 | 98655832 | OT129 | ex2 SA-2 |
| chr7 | 99966974 | 99967023 | OT130 | ex2 SA-3 |
| chr7 | 100747515 | 100747584 | OT131 | ex2 SA-3 |
| chr7 | 106472738 | 106472798 | OT132 | ex2 SP |
| chr7 | 107152042 | 107152111 | OT133 | ex2 SA-1 |
| chr7 | 119404360 | 119404432 | OT134 | ex2 SA-2; ex2 SA-1; ex2 SA-3 |
| chr7 | 119404361 | 119404432 | OT135 | ex2 SA-2; ex2 SA-1; ex2 SA-3 |
| chr7 | 119404362 | 119404432 | OT136 | ex2 SA-2; ex2 SA-1; ex2 SA-3 |
| chr7 | 138075555 | 138075602 | OT137 | ex2 SA-3 |
| chr8 | 6637972 | 6638045 | OT138 | ex2 SA-2; ex2 SA-1; ex2 SA-3 |
| chr8 | 6637973 | 6638045 | OT139 | ex2 SA-2; ex2 SA-1; ex2 SA-3 |
| chr8 | 6637974 | 6638045 | OT140 | ex2 SA-2; ex2 SA-1; ex2 SA-3 |
| chr8 | 11627499 | 11627564 | OT141 | ex2 SA-3 |
| chr8 | 29365092 | 29365156 | OT142 | ex2 SA-2; ex2 SA-1 |
| chr8 | 29365093 | 29365156 | OT143 | ex2 SA-2; ex2 SA-1 |
| chr8 | 43940707 | 43940754 | OT144 | ex2 SP |
| chr8 | 45946908 | 45946955 | OT145 | ex2 SP |
| chr8 | 52375463 | 52375510 | OT146 | ex2 SA-3 |
| chr8 | 60632176 | 60632235 | OT147 | ex2 SP |
| chr8 | 127587658 | 127587712 | OT148 | ex2 SA-1 |
| chr8 | 136675834 | 136675902 | OT149 | ex2 SP |
| chr8 | 143608104 | 143608172 | OT150 | ex2 SA-2 |
| chr9 | 846943 | 847002 | OT151 | ex2 SA-3 |
| chr9 | 5557152 | 5557212 | OT152 | ex2 SA-1 |
| chr9 | 10684980 | 10685043 | OT153 | ex2 SA-3 |
| chr9 | 18483096 | 18483166 | OT154 | ex2 SP |
| chr9 | 78945328 | 78945401 | OT155 | ex2 SA-1 |
| chr9 | 93689848 | 93689916 | OT156 | ex2 SP |
| chr9 | 133703244 | 133703294 | OT157 | ex2 SA-3 |
| chr9 | 134910961 | 134911034 | OT158 | ex2 SA-2; ex2 SA-1 |
| chr9 | 134910962 | 134911034 | OT159 | ex2 SA-2; ex2 SA-1 |
| chr9 | 136697152 | 136697213 | OT160 | ex2 SA-3 |
| chr9 | 137954061 | 137954106 | OT161 | ex2 SA-3 |
| chr10 | 16068828 | 16068895 | OT162 | ex2 SA-1 |
| chr10 | 16832580 | 16832636 | OT163 | ex2 SA-1 |
| chr10 | 28717805 | 28717856 | OT164 | ex2 SA-3 |
| chr10 | 35212659 | 35212714 | OT165 | ex2 SP |
| chr10 | 39530828 | 39530875 | OT166 | ex2 SP |
| chr10 | 80504729 | 80504799 | OT167 | ex2 SP |
| chr10 | 86255576 | 86255644 | OT168 | ex2 SP |
| chr10 | 107641621 | 107641665 | OT169 | ex2 SP |
| chr10 | 122980788 | 122980833 | OT170 | |
| chr10 | 130431503 | 130431561 | OT171 | ex2 SA-3 |
| chr11 | 2764027 | 2764077 | OT172 | ex2 SA-2; ex2 SA-1; ex2 SA-3 |
| chr11 | 2764027 | 2764078 | OT173 | ex2 SA-2; ex2 SA-1; ex2 SA-3 |
| chr11 | 2764027 | 2764079 | OT174 | ex2 SA-2; ex2 SA-1; ex2 SA-3 |
| chr11 | 34183107 | 34183164 | OT175 | ex2 SA-1 |
| chr11 | 34901517 | 34901588 | OT176 | ex2 SA-2; ex2 SA-1 |
| chr11 | 34901517 | 34901589 | OT177 | ex2 SA-2; ex2 SA-1 |
| chr11 | 43803703 | 43803766 | OT178 | ex2 SA-2 |
| chr11 | 44886174 | 44886242 | OT179 | ex2 SA-1 |
| chr11 | 47958768 | 47958828 | OT180 | ex2 SA-1 |
| chr11 | 76341287 | 76341348 | OT181 | ex2 SA-2 |
| chr11 | 78200640 | 78200690 | OT182 | ex2 SA-2; ex2 SA-3 |
| chr11 | 78200640 | 78200691 | OT183 | ex2 SA-2; ex2 SA-3 |
| chr11 | 86112455 | 86112521 | OT184 | ex2 SA-3 |
| chr11 | 100943070 | 100943114 | OT185 | ex2 SA-2; ex2 SA-1 |
| chr11 | 100943071 | 100943114 | OT186 | ex2 SA-2; ex2 SA-1 |
| chr11 | 116139590 | 116139652 | OT187 | ex2 SA-3 |
| chr11 | 122623892 | 122623940 | OT188 | ex2 SA-3 |
| chr12 | 30847131 | 30847204 | OT189 | ex2 SA-2 |
| chr12 | 65527337 | 65527389 | OT190 | ex2 SA-3 |
| chr12 | 65881308 | 65881354 | OT191 | ex2 SP |
| chr12 | 67120945 | 67121000 | OT192 | ex2 SA-3 |
| chr12 | 90057206 | 90057270 | OT193 | ex2 SA-2; ex2 SA-1 |
| chr12 | 90057206 | 90057271 | OT194 | ex2 SA-2; ex2 SA-1 |
| chr12 | 117482278 | 117482326 | OT195 | ex2 SA-2 |
| chr12 | 125238097 | 125238151 | OT196 | ex2 SA-2; ex2 SA-3 |
| chr12 | 125238097 | 125238152 | OT197 | ex2 SA-2; ex2 SA-3 |
| chr13 | 24995160 | 24995233 | OT198 | ex2 SA-2 |
| chr13 | 33022366 | 33022413 | OT199 | ex2 SP |
| chr13 | 37275360 | 37275427 | OT200 | ex2 SP |
| chr13 | 46166460 | 46166519 | OT201 | ex2 SA-2; ex2 SA-1; ex2 SA-3 |
| chr13 | 46166460 | 46166520 | OT202 | ex2 SA-2; ex2 SA-1; ex2 SA-3 |
| chr13 | 46166460 | 46166521 | OT203 | ex2 SA-2; ex2 SA-1; ex2 SA-3 |
| chr13 | 88648207 | 88648258 | OT204 | ex2 SA-2 |
| chr13 | 97920356 | 97920427 | OT205 | ex2 SA-2; ex2 SA-1; ex2 SA-3 |
| chr13 | 97920356 | 97920428 | OT206 | ex2 SA-2; ex2 SA-1; ex2 SA-3 |
| chr13 | 97920356 | 97920429 | OT207 | ex2 SA-2; ex2 SA-1; ex2 SA-3 |
| chr14 | 50599875 | 50599924 | OT208 | ex2 SA-3 |
| chr14 | 68761493 | 68761536 | OT209 | ex2 SA-2; ex2 SA-1; ex2 SA-3 |
| chr14 | 68761493 | 68761537 | OT210 | ex2 SA-2; ex2 SA-1; ex2 SA-3 |
| chr14 | 68761493 | 68761538 | OT211 | ex2 SA-2; ex2 SA-1; ex2 SA-3 |
| chr14 | 98014791 | 98014847 | OT212 | ex2 SA-2 |
| chr14 | 101958211 | 101958270 | OT213 | ex2 SA-1 |
| chr15 | 24980160 | 24980230 | OT214 | ex2 SA-1 |
| chr15 | 39747311 | 39747358 | OT215 | ex2 SA-3 |
| chr15 | 57589759 | 57589809 | OT216 | ex2 SA-1 |
| chr15 | 62370716 | 62370775 | OT217 | ex2 SP |
| chr15 | 66657688 | 66657732 | OT218 | ex2 SA-2; ex2 SA-3 |
| chr15 | 66657688 | 66657733 | OT219 | ex2 SA-2; ex2 SA-3 |
| chr15 | 89486335 | 89486384 | OT220 | ex2 SA-2; ex2 SA-1; ex2 SA-3 |
| chr15 | 89486335 | 89486385 | OT221 | ex2 SA-2; ex2 SA-1; ex2 SA-3 |
| chr15 | 89486335 | 89486386 | OT222 | ex2 SA-2; ex2 SA-1; ex2 SA-3 |
| chr16 | 17071340 | 17071394 | OT223 | |
| chr16 | 28865277 | 28865326 | OT224 | ex2 SA-3 |
| chr16 | 34209005 | 34209052 | OT225 | ex2 SP |
| chr16 | 67090053 | 67090118 | OT226 | ex2 SP |
| chr17 | 6219108 | 6219163 | OT227 | ex2 SA-2 |
| chr17 | 16700359 | 16700412 | OT228 | ex2 SA-2; ex2 SA-1; ex2 SA-3 |
| chr17 | 16700359 | 16700413 | OT229 | ex2 SA-2; ex2 SA-1; ex2 SA-3 |
| chr17 | 16700359 | 16700414 | OT230 | ex2 SA-2; ex2 SA-1; ex2 SA-3 |
| chr17 | 18615025 | 18615080 | OT231 | ex2 SA-2; ex2 SA-1; ex2 SA-3 |
| chr17 | 18615026 | 18615080 | OT232 | ex2 SA-2; ex2 SA-1; ex2 SA-3 |
| chr17 | 18615027 | 18615080 | OT233 | ex2 SA-2; ex2 SA-1; ex2 SA-3 |
| chr17 | 18830922 | 18830975 | OT234 | ex2 SA-2; ex2 SA-1; ex2 SA-3 |
| chr17 | 18830922 | 18830976 | OT235 | ex2 SA-2; ex2 SA-1; ex2 SA-3 |
| chr17 | 18830922 | 18830977 | OT236 | ex2 SA-2; ex2 SA-1; ex2 SA-3 |
| chr17 | 27534883 | 27534931 | OT237 | ex2 SA-2; ex2 SA-1; ex2 SA-3 |
| chr17 | 27534883 | 27534932 | OT238 | ex2 SA-2; ex2 SA-1; ex2 SA-3 |
| chr17 | 27534883 | 27534933 | OT239 | ex2 SA-2; ex2 SA-1; ex2 SA-3 |
| chr17 | 29960178 | 29960223 | OT240 | ex2 SA-2 |
| chr17 | 32651484 | 32651528 | OT241 | ex2 SA-1 |
| chr17 | 34196890 | 34196943 | OT242 | |
| chr17 | 56341318 | 56341362 | OT243 | ex2 SA-3 |
| chr17 | 65636924 | 65636968 | OT244 | ex2 SA-3 |
| chr17 | 72391913 | 72391966 | OT245 | ex2 SA-2; ex2 SA-3 |
| chr17 | 72391914 | 72391966 | OT246 | ex2 SA-2; ex2 SA-3 |
| chr17 | 74012781 | 74012832 | OT247 | ex2 SA-3 |
| chr18 | 3042161 | 3042233 | OT248 | ex2 SA-2 |
| chr18 | 3585454 | 3585526 | OT249 | ex2 SA-2; ex2 SA-3 |
| chr18 | 3585454 | 3585527 | OT250 | ex2 SA-2; ex2 SA-3 |
| chr18 | 11762075 | 11762130 | OT251 | ex2 SA-2; ex2 SA-1 |
| chr18 | 11762076 | 11762130 | OT252 | ex2 SA-2; ex2 SA-1 |
| chr18 | 25408504 | 25408569 | OT253 | ex2 SA-2 |
| chr18 | 30933038 | 30933105 | OT254 | ex2 SA-2; ex2 SA-3 |
| chr18 | 30933038 | 30933106 | OT255 | ex2 SA-2; ex2 SA-3 |
| chr18 | 72360200 | 72360251 | OT256 | ex2 SA-2; ex2 SA-1; ex2 SA-3 |
| chr18 | 72360200 | 72360252 | OT257 | ex2 SA-2; ex2 SA-1; ex2 SA-3 |
| chr18 | 72360200 | 72360253 | OT258 | ex2 SA-2; ex2 SA-1; ex2 SA-3 |
| chr18 | 73831275 | 73831342 | OT259 | ex2 SA-2; ex2 SA-3 |
| chr18 | 73831275 | 73831343 | OT260 | ex2 SA-2; ex2 SA-3 |
| chr18 | 76059333 | 76059388 | OT261 | ex2 SP |
| chr19 | 17778639 | 17778702 | OT262 | ex2 SA-3 |
| chr19 | 24391201 | 24391247 | OT263 | ex2 SP |
| chr19 | 24640055 | 24640101 | OT264 | ex2 SP |
| chr19 | 24863033 | 24863079 | OT265 | ex2 SP |
| chr19 | 27879658 | 27879729 | OT266 | ex2 SA-2; ex2 SA-1; ex2 SA-3 |
| chr19 | 27879658 | 27879730 | OT267 | ex2 SA-2; ex2 SA-1; ex2 SA-3 |
| chr19 | 27879658 | 27879731 | OT268 | ex2 SA-2; ex2 SA-1; ex2 SA-3 |
| chr19 | 37779806 | 37779860 | OT269 | ex2 SA-3 |
| chr19 | 39481148 | 39481202 | OT270 | ex2 SA-2 |
| chr19 | 53129659 | 53129705 | OT271 | ex2 SA-3 |
| chr20 | 13400270 | 13400328 | OT272 | ex2 SA-3 |
| chr20 | 29214318 | 29214365 | OT273 | ex2 SP |
| chr20 | 58957385 | 58957435 | OT274 | ex2 SP |
| chr21 | 26272964 | 26273033 | OT275 | ex2 SA-2; ex2 SA-1 |
| chr21 | 26272964 | 26273034 | OT276 | ex2 SA-2; ex2 SA-1 |
| chr21 | 33639196 | 33639269 | OT277 | ex2 SA-3 |
| chr21 | 33899952 | 33900024 | OT278 | ex2 SP |
| chr21 | 36159758 | 36159815 | OT279 | ex2 SA-3 |
| chr22 | 22557738 | 22557799 | OT280 | |
| chr22 | 32541679 | 32541737 | OT281 | ex2 SA-3 |
| chr22 | 32603658 | 32603726 | OT282 | ex2 SA-1 |
| chr22 | 50175411 | 50175476 | OT283 | ex2 SA-2; ex2 SA-1 |
| chr22 | 50175411 | 50175477 | OT284 | ex2 SA-2; ex2 SA-1 |
| chrX | 16678458 | 16678516 | OT285 | ex2 SP |
| chrX | 22905893 | 22905938 | OT286 | ex2 SA-2; ex2 SA-1; ex2 SA-3 |
| chrX | 22905893 | 22905939 | OT287 | ex2 SA-2; ex2 SA-1; ex2 SA-3 |
| chrX | 22905893 | 22905940 | OT288 | ex2 SA-2; ex2 SA-1; ex2 SA-3 |
| chrX | 23097708 | 23097769 | OT289 | ex2 SP |
| chrX | 35413798 | 35413865 | OT290 | ex2 SA-1 |
| chrX | 46272094 | 46272142 | OT291 | ex2 SP |
| chrX | 58624495 | 58624542 | OT292 | ex2 SP |
| chrX | 59418465 | 59418512 | OT293 | ex2 SP |
| chrX | 60442200 | 60442247 | OT294 | ex2 SP |
| chrX | 60965797 | 60965844 | OT295 | ex2 SP |
| chrX | 62005522 | 62005569 | OT296 | ex2 SP |
| chrX | 72801971 | 72802038 | OT297 | ex2 SA-1 |
| chrX | 72918785 | 72918852 | OT298 | ex2 SA-1 |
| chrX | 74131144 | 74131190 | OT299 | ex2 SP |
| chrX | 107785841 | 107785894 | OT300 | ex2 SA-2; ex2 SA-3 |
| chrX | 107785842 | 107785894 | OT301 | ex2 SA-2; ex2 SA-3 |
| chrX | 117087804 | 117087872 | OT302 | ex2 SP |
| chrX | 125437231 | 125437304 | OT303 | ex2 SA-2 |
| chrX | 143474293 | 143474349 | OT304 | ex2 SA-1 |
| chrX | 146151607 | 146151668 | OT305 | ex2 SA-2; ex2 SA-1 |
| chrX | 146151608 | 146151668 | OT306 | ex2 SA-2; ex2 SA-1 |
| chrX | 153535528 | 153535595 | OT307 | ex2 SA-1 |

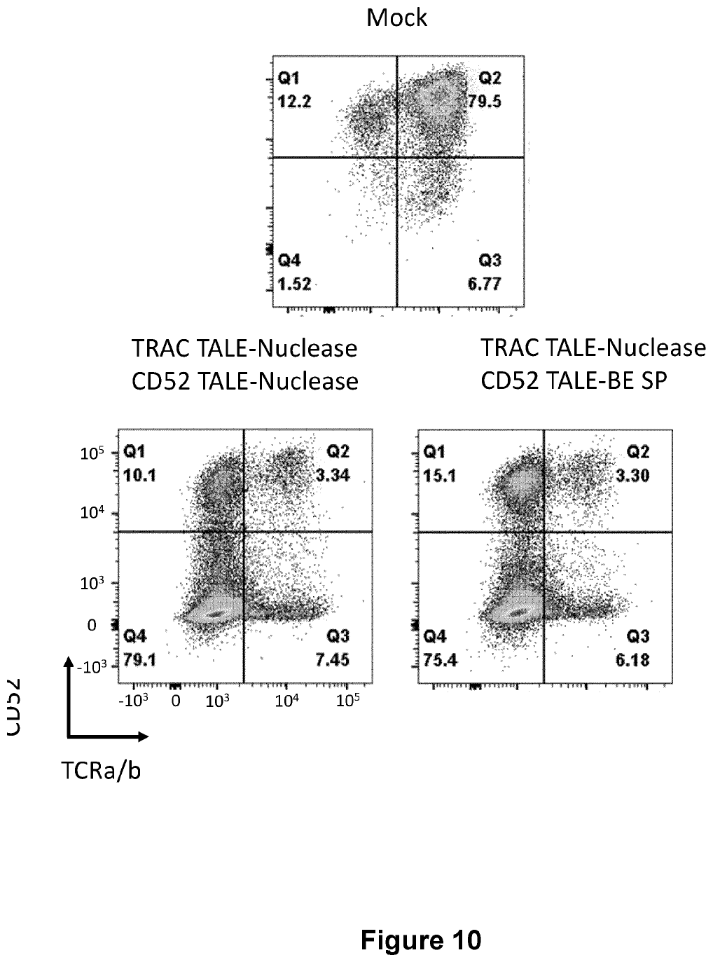

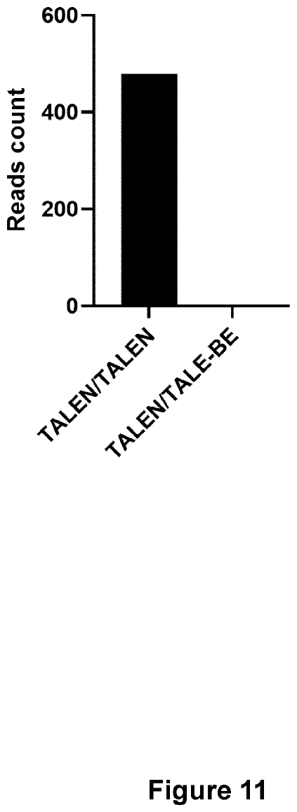

Finally, as the TALEB 0052 splice site BE only created marginal levels of Indels, we hypothesized that multiplex gene editing (i.e. simultaneous use of a base editor and a nuclease, such as a TALE-Nuclease) should not create chromosome translocations, a phenomenon commonly observed in cells treated with multiple nucleases. As a proof of concept, a TALE-Nuclease targeting TRAC ((SEQ ID NO:16 and SEQ ID NO:17) was combined with either a 0052 TALE-Nuclease (SEQ ID NO:18 and SEQ ID NO:19) or the base editor TALE-BE SP (SEQ ID NO:26 and SEQ ID NO:27). While high and similar levels of phenotypic double gene KO were detected by flow cytometry in both TALE-Nuclease/TALE-Nuclease and TALE-Nuclease/TALE base editor treated cells (79% and 75% respectively, FIG. 10), translocation (as measured by multiplexed amplicon sequencing) between the two targeted loci were only observed in TALE-Nuclease/TALE-Nuclease treated sample (479 reads out of 224,406 for the TALE-Nuclease/TALE-Nuclease sample and 0 reads out of 144,323 for the TALE-Nuclease/BE sample, N=1, 1 single T-cell donor, see diagram of FIG. 11).

### Discussion

Base editing represents one of the newest gene editing technologies. Recently, the TALE scaffold was demonstrated to be compatible with the creation of a new class of DddA-derived cytosine base editors. In the above experimental study, the screening of several base editors targeting various endogenous loci with the development of a simple and robust medium-throughput approach has been carried out to investigate the determinants of editing by TALE-base editors. This throughput screening strategy has taken advantage of the highly efficient and precise TALE-nuclease mediated ssODN knock-in in primary T cells and allowed to assess the TALE base editor editing efficiency on hundreds of different targets in cellulo. Because all base editor artificial target sequences were inserted into the same predefined locus in the genome, this method allowed to focus on how target/spacer sequence variations could affect TALE base editors while excluding factors such as DNA binding affinities or epigenetic variations. The experimental results pointed out an optimal 13-17 bp spacer length window for editing, with the G1397C-bearing arm of the TALE base editors being placed 4-7 bp down the 3′ direction of the target TC for the best editing activity.

While extremely precise introduction of the intended mutation (high purity of the final product) is a prerequisite for application such as gene correction, generation of DSBs by base editors may raise greatest concerns, especially since CRISPR/Cas base nucleases have been recently associated with major on-target genome instability or chromosomal abnormalities [Weisheit, et al. (2020). Detection of Deleterious On-Target Effects after HDR-Mediated CRISPR Editing. Cell Rep. 31. Boutin, J., et al. (2022). ON-Target Adverse Events of CRISPR-Cas9 Nuclease: More Chaotic than Expected. Cris. J. 5, 19-30]. In this study, only marginal byproduct mutation (C-to-A/G) have been detected, and more importantly low Indel creation, by TALE base editors looking at dozens of these molecular tools, even at high editing frequencies (>80% in bulk population). However, a careful design of the base editors positioning, allowed to prevent or minimize bystander mutations.

Base editors have been used to edit or mutate conserved genetic elements such as enhancers [Zeng, J., et al. (2020). Therapeutic base editing of human hematopoietic stem cells. Nat. Med. 26, 535-541], start codons, splice sites [Kluesner, M. G et al. (2021). CRISPR-Cas9 cytidine and adenosine base editing of splice-sites mediates highly-efficient disruption of proteins in primary and immortalized cells. Nat. Commun. 12:1-12], branch points and conserved active sites [Hanna, R. E., et al. (2021). Massively parallel assessment of human variants with base editor screens. Cell 184, 1064-1080]. It has been estimated that ˜46,000 (46,608) splice sites in the genome could potentially be targeted by TALE base editors as per the present invention, impacting 15,279 different transcripts, representing 76.57% of all the transcripts in human genome and, overall, indicating that splice site editing could be a viable approach for gene knock-out by TALE base editors. To demonstrate the feasibility of such an approach, highly efficient TALE base editors have been designed targeting the conserved G of the intron 1/exon 2 junction splice site of the CD52 gene. It was also demonstrated that, as an alternative to splice site editing, targeting the signal peptide can also lead to efficient surface CD52 protein knock-out.

Thus, base editors represent promising molecular tools for multiplex gene engineering, though they have been so far limited to knock-out or gene corrections. Here, it has been demonstrated the feasibility of efficient multiplex gene engineering using a combination of two different molecular tools, a nuclease, and a base editor. Such a multiplex/multitool strategy presents several advantages. First, it prevents creation of translocations often observed with the simultaneous use of several (>1) nucleases, and second, it allows the possibility to go beyond multiple knock-outs, while still allowing gene knock-in at the nuclease target site, altogether extending the scope of possible application, while better controlling the engineered cell population outcome (e.g. absence of translocations). The precise positional rules that have been hereby determined for TALE base editors allow lower frequency of unwanted indels generation, and increased accessibility to additional cell compartments beyond the traditional nuclear targets. They thereby expand the potential scope of TALE-based multiplex/multitool strategy beyond the capabilities of most other non-TALE editing tools.

### Example 5: Application to Gene Therapy to Correct Exon 24 of PIK3CD Gene that Causes Combined Immunodeficiency ADPS1

The methods of the invention described herein aim at improving the efficiency and safety of TALEN-mediated therapeutic gene insertion in long-term Hematopoietic Stem Cell (LT-HSC) of individuals affected by a dominant negative genetic disease. The treatment consists in the TALEN-mediated insertion of a therapeutic repair matrix (cDNA of the mutated gene) in the introns or exons of the faulty gene, followed by the TALE Base editor-mediated inactivation of the same faulty gene. The TALE Base editor treatment proposed by this method could theoretically increase the frequency of cells harboring a normal phenotype without creating additional genomic adverse events due to the simultaneous creation of double strand break. Overall, inactivation of the remaining faulty gene is supposed to improve the therapeutic outcome the gene therapy intervention.

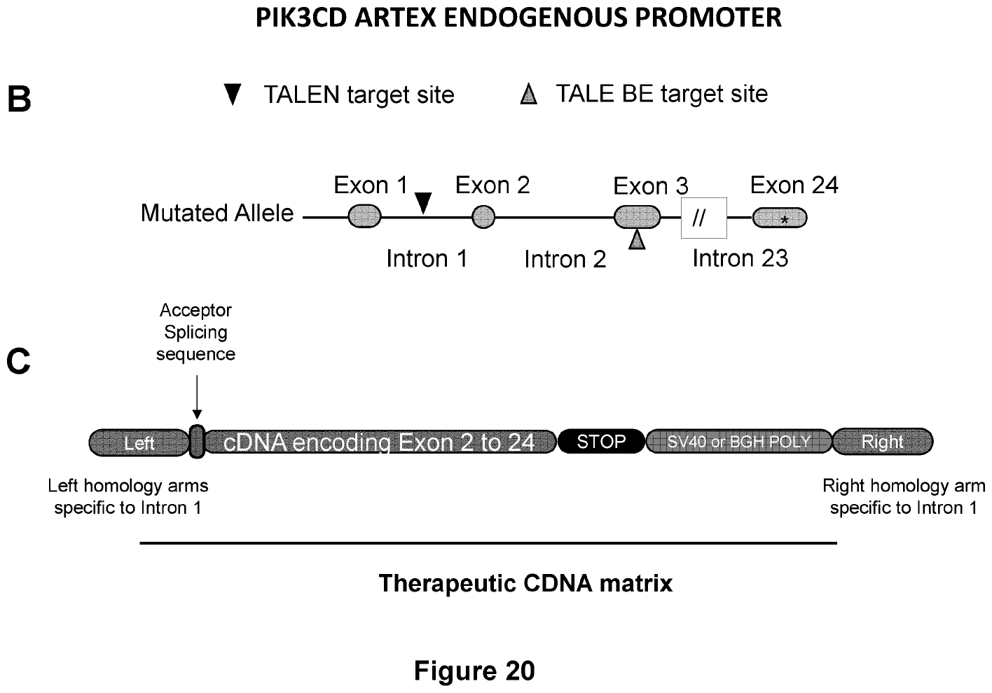

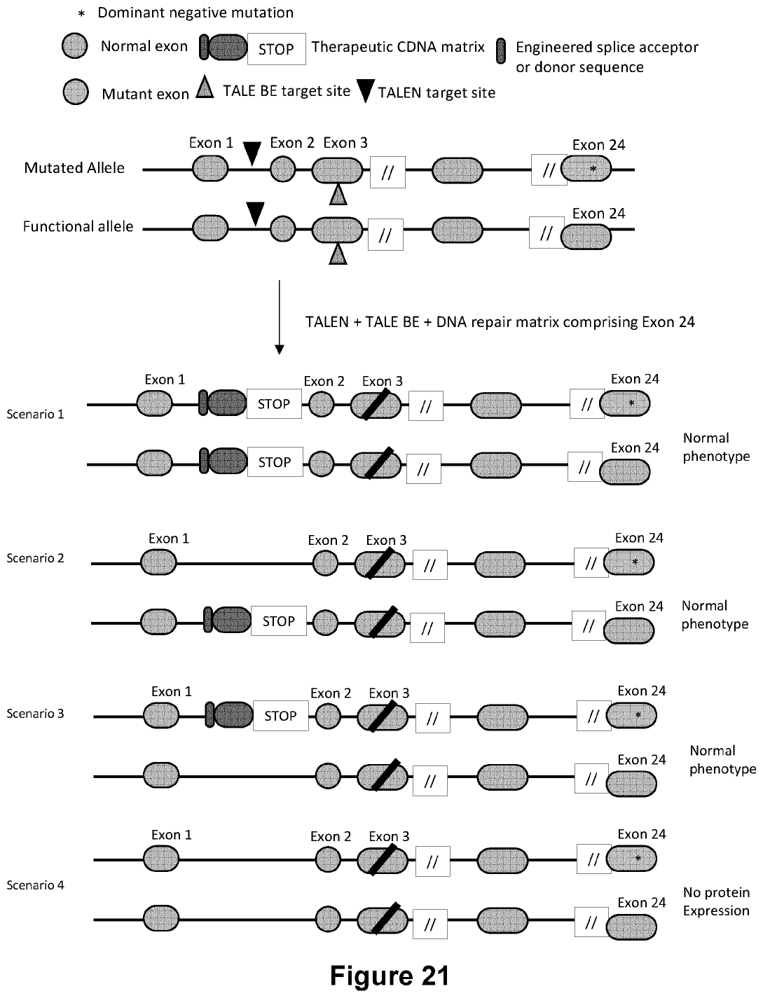

APDS1 is a combined immunodeficiency caused by a gain-of-function mutations (E1021K) occurring in the exon 24 of the PIK3CD gene. This indication can benefit from the TALEN/TALE Base editor mediated targeted repair approach, which principles are described in FIGS. 12 to 16 (Artex integration of rewritten PIK3CD corrected sequence+inactivation of downstream original exons by using base editors). Such TALEN/TALE Base editor mediated targeted repair/inactivation approach with respect to exon 24 of PIK3CD is illustrated below in FIGS. 20 and 21. The treatment of APDS1 cells with a TALEN targeting the Intron 1 (between Exon 1 and 2) promotes the insertion of a re-encoded therapeutic cDNA matrix carrying the correct version of PIK3CD cDNA (from Exon 2 to Exon 24). A simultaneous treatment by a TALE Base Editor targeting the Exon 3 (see selection of TALE Base Editor target sites in table 12) creates stop codons downstream the therapeutic cassette insertion site and thus prevent the mutated allele to be expressed.

### Example 6: Influence of the Spacer Length on CO, C11, C40 C-to-T Editing Efficiency

TALE base editor heterodimer is a double strand bacterial deaminase characterized by the fusion of: 1) catalytic domain split in two inactive halves that, upon reconstitution, will catalyze the conversion of a cytosine (C) to a thymine (T); 2) transcription activator-like effector domain (TALE) for DNA binding and 3) an uracil glycosylase inhibitor (UGI) (Mok B. Y. et al., Nature 2020). These TALE base editors have been used for several applications including the creation of mutations in mitochondrial DNA mitochondria (Mok B. Y. et al., Nature 2020) or in chloroplast (Beum-Chang Kang, et al., Nature Plants 2021). Despite these successful applications, the editing rules and target sequence specificities of the TALE base editors are still limited. More detailed and comprehensive study are therefore necessary to create further TALE base editor generations. However, such progress is challenging. In vitro studies require purified recombinant TALE base editors and cell-based approaches are tedious because as many different TALE targeting various loci would be required to rule out confounding effects such as epigenomic factors or modification.

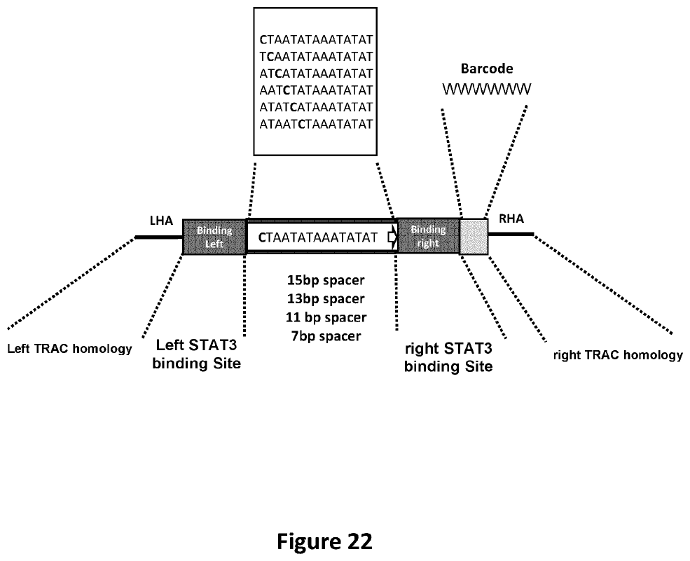

To define the key determinants for efficient TALE base editing (C-to-T conversion) in function of the reported preferred 5′-TC position within the 15 bp spacer length/editing window (FIG. 1), the inventors have set up a medium to high throughput format screening, in a define genomic context, which has been designed by generating a pool of primary T-cells, containing predefined TALE base editor target sequences precisely inserted at the TRAC gene (FIG. 5). Each of the TALE base editors targets containing a unique TC or GA (target for the DddA deaminase) within the spacer sequence flanked by two fixed TALE binding sequences (RVD-L and RVD-R, FIG. 22). This setup allows the uniform TALE base editor binding to the artificial target sites, excluding editing variability caused by different DNA binding affinities from different TALE array protein and the impact of epigenomic factors, such as chromosome relaxation around the artificial BE target sites.

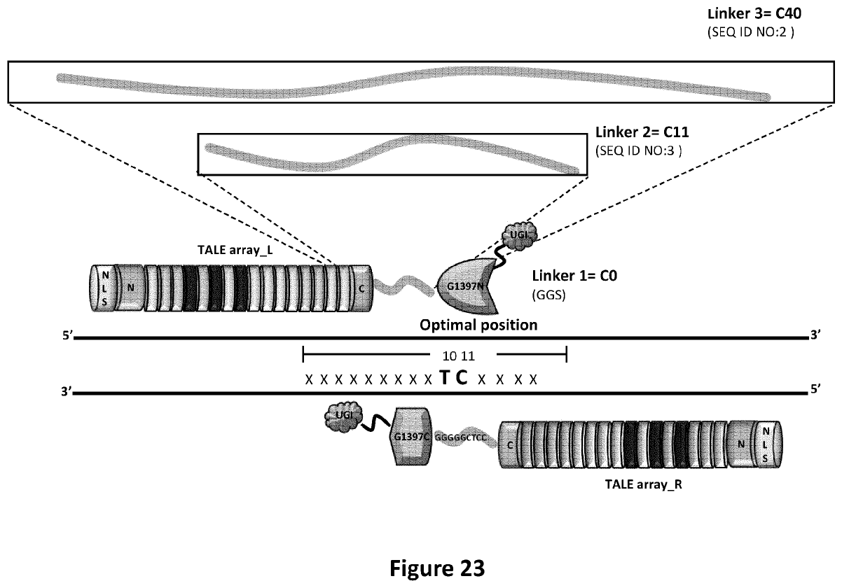

To investigate whether the length of the linker that connect the TAL array with the split head on both arms, could potentially impact the movement of DddA head splits, and so change the target specificity, STAT3 TALE base editors were constructed with different TALE C-terminal lengths referred to as C40, C11 and CO backbones (Table 13, FIG. 23).

TABLE 13
TALE C-terminus used in C40, C11 and C0 TALEB
scaffolds in example 6.

| | | SEQ |
|---|---|---|
| TALE | | ID |
| C-terminus | Amino acid sequence | NO# |
| C40 | SIVAQLSRPDPALAALTNDHLVALACLGGR | 551 |
| | PALDAVKKGLGGS | |
| C11 | SIVAQLSRPDPSGSGSGGGS | 552 |
| CO | GGS | N.A. |

### Influence of the Spacer Length (CO, C11 and C40) on C-to-T Editing Efficiency

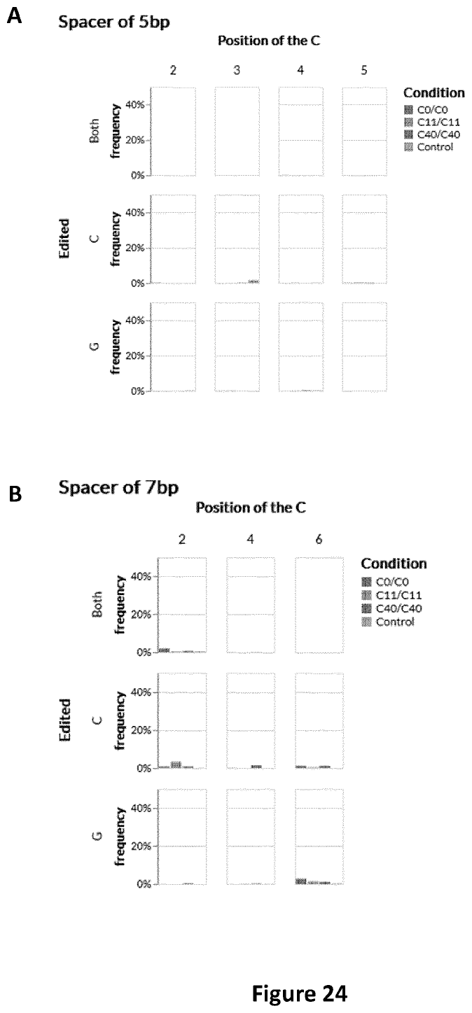

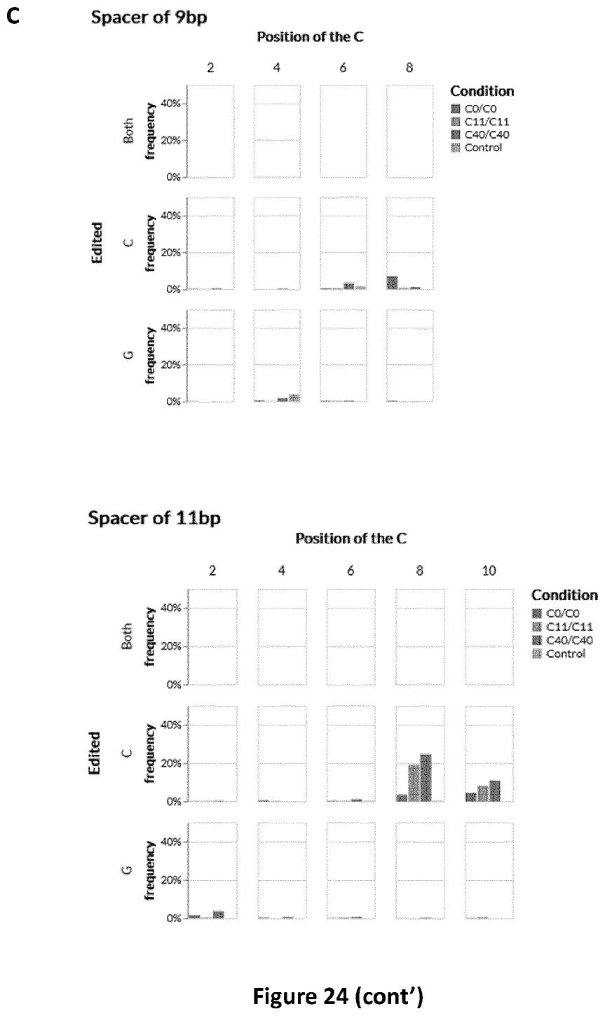

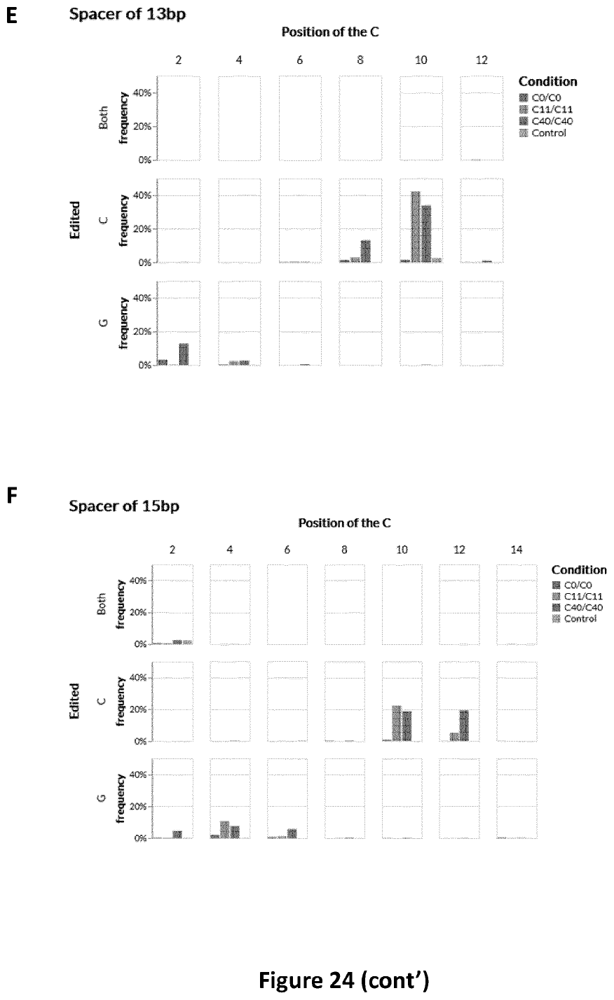

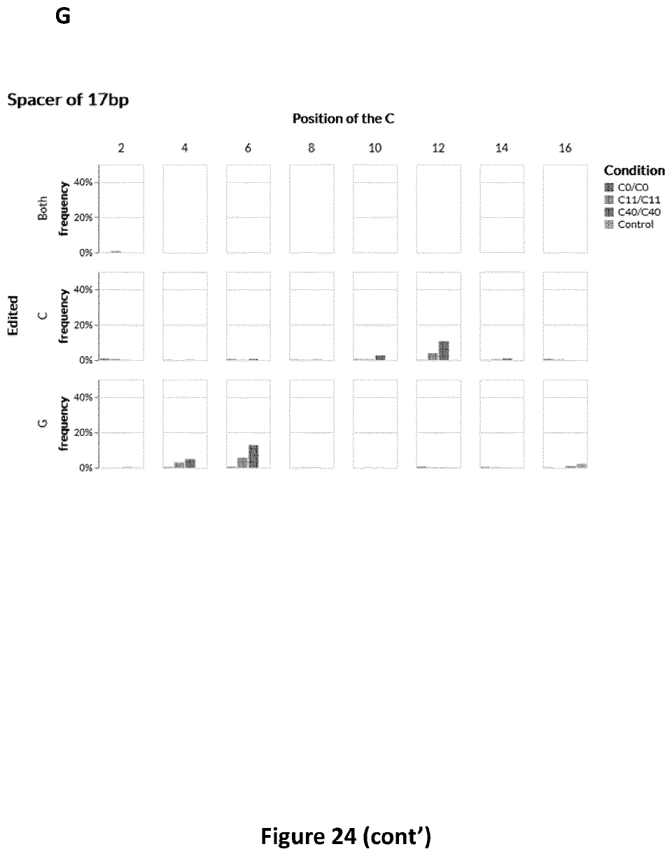

A collection of ssODN that contain two fixed TALE array protein binding sites from the STAT3 TALE base editors separated by spacers with various number length were constructed as shown in FIG. 24, spanning from 5 to 17 bp (i.e. 5, 7, 9, 11 . . . 17 bp) to evaluate differences related to spacer length within the STAT3 target sequence. A TCGA quadruplex target sequence was incorporated in the spacer at every other position to generate the pool of primary T-cells harboring the collection of BE targets. Additionally, to facilitate the sequence analysis, a unique barcode was added to each construct. The resulting 37 unique ssODNs (Table 15) were mixed in equal amount and transfected in primary T-cells by electroporation (200 pmol per million cells) simultaneously with mRNA encoding the TALE-Nuclease targeting TRAC (SEQ ID NO:16 and 17). In a second step, two days post transfection of the TRAC TALEN and ssODN pool, the mRNAs encoding STAT3 TALE base editors (mixed linkers length) were co-electroporated.

TABLE 14
TALEB heterodimer structures tested in Example 6

| | | left/right |
|---|---|---|
| TALEB (1 ug/left arm) | TALEB (1 ug/right arm) | C- |
| plasmid references | plasmid references | terminus |
| pCLS36448 | pCLS36495 | C40/C40 |
| (encoding SEQ ID NO: 553) | (encoding SEQ ID NO: 554) | |
| pCLS37657 | pCLS37680 | C11/C11 |
| (encoding SEQ ID NO: 557) | (encoding SEQ ID NO: 558) | |
| pCLS37610 | pCLS37633 | C0/C0 |
| (encoding SEQ ID NO: 554) | (encoding SEQ ID NO: 556) | |
| pCLS36448 | pCLS37680 | C40/C11 |
| (encoding SEQ ID NO: 553) | (encoding SEQ ID NO: 558) | |
| pCLS37657 | pCLS36495 | C11/C40 |
| (encoding SEQ ID NO: 557) | (encoding SEQ ID NO: 554) | |

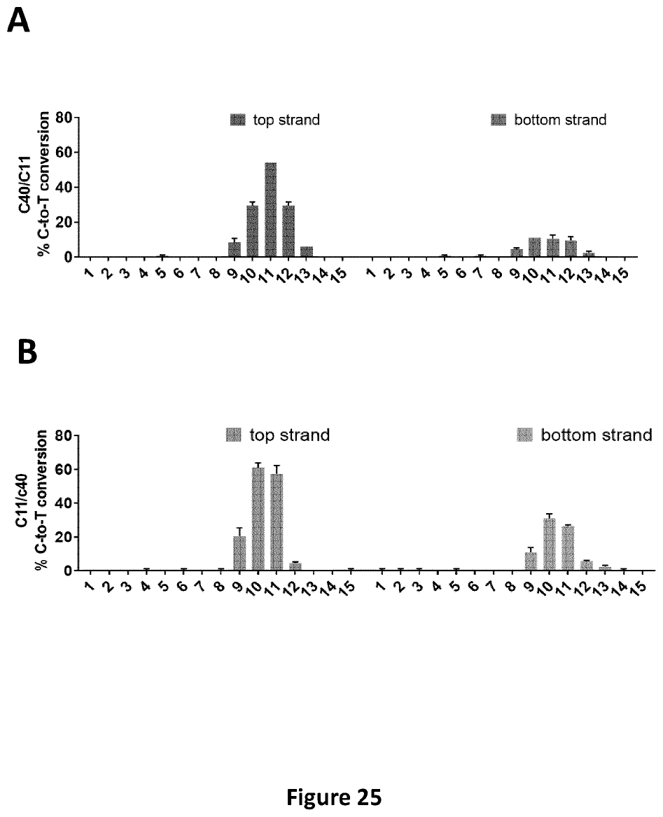

The genomic DNA of transfected cells was then harvested at day 2 post TALE base editor transfection for editing analysis. The NGS analysis data as compiled and represented in the diagrams of FIG. 24 (C11/C11 and C40/C40 TALEB heterodimers) and FIG. 25 (mixed C11/C40 and C40/C11 TALEB heterodimers) showed that:
the spacer is best edited when between 11 bp (iv) and 15 bp long (vi), with a maximum at 13 bp (v);
the edition is generally better with C11/C11, closely followed by C40/C40;
the position of the C/G and the size of the spacer remain the most influential parameters on the editing efficiency.

### Influence of the Context Around TC: 15 bp Spacer Length.

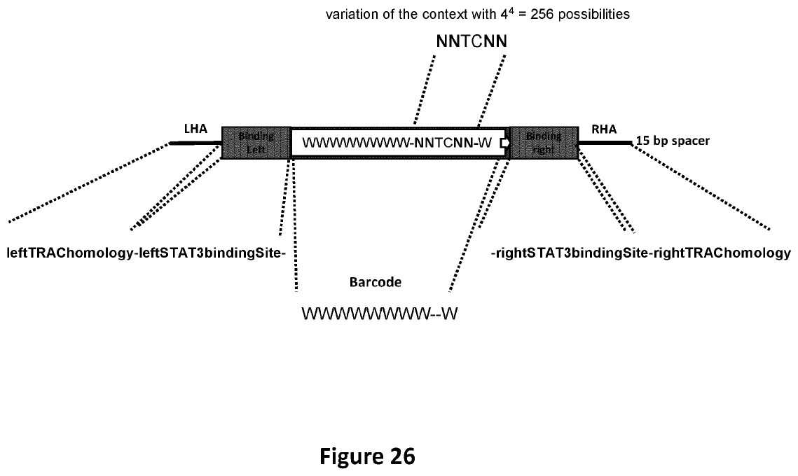

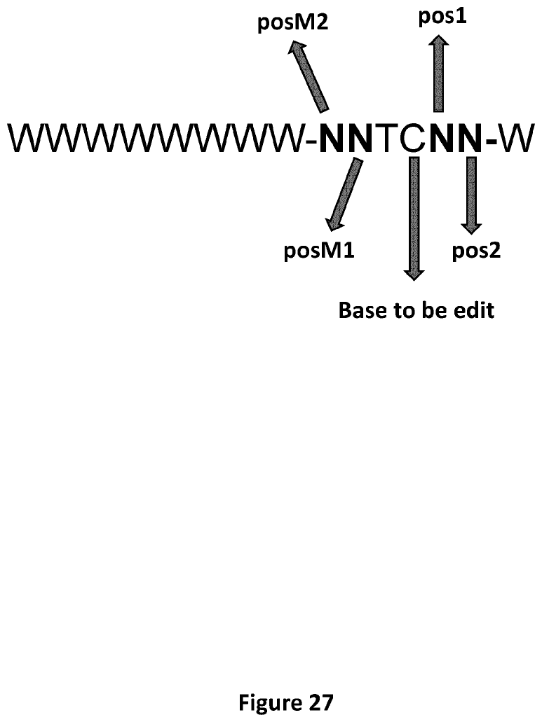

In order to evaluate the effect of the surrounding context on TALEB editing efficiency within the 15 bp spacer length, libraries comprising 256 unique ssODN were designed, as represented in FIGS. 26 and 27 and detailed in Table 16). PBMCs from 2 donors were transfected with TRAC TALEN and the three different pools of oligos to be inserted in the TRAC locus, followed by either STAT3 BE C40/C40 or C11/C11 transfection for editing of the cells with oligo KI. gDNA was made from cells treated with the three oligo pools, and samples were sent for sequencing on MiSeq.

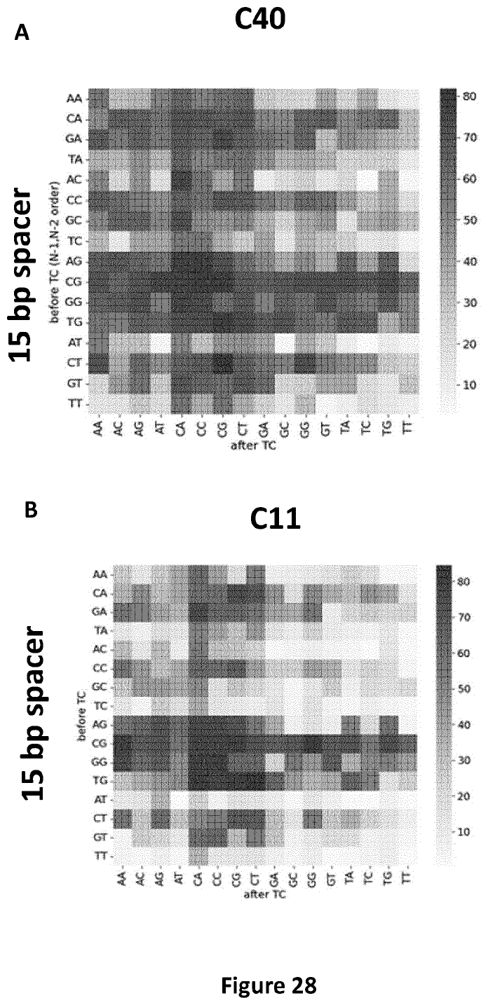

Data analysis from bioinformatics determined the contribution of each surrounding base to the efficiency of C editing as represented in FIGS. 28 (A and B), which was found to be similar for both architectures:

$$\begin{matrix}at position M 2 : A \leq T ≪ G < C . \\ at position M 1 : T \leq C < A ≪ G . \\ at position 1 : T \leq G < A ≪ C \\ at position 2 : T < C < G ≪ A\end{matrix}$$

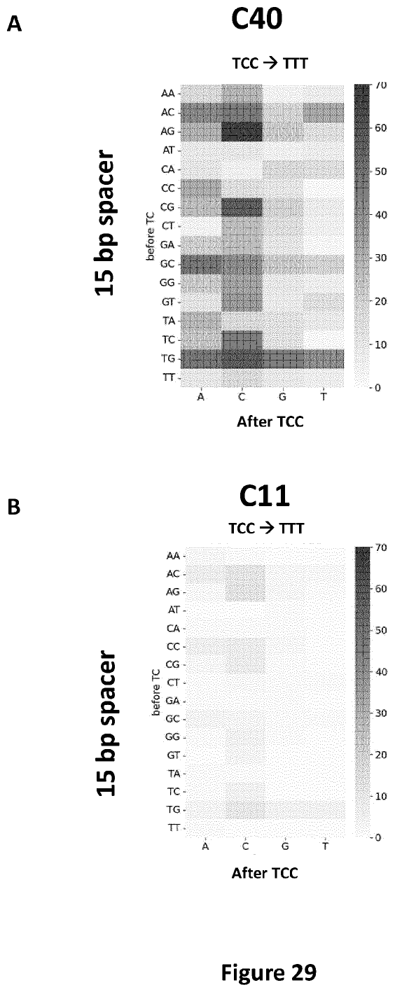

We also looked at multiple editions where Cs do follow the central TC (TCC to TTT) and editing analysis showed that C40 architecture is more tolerant than C11 (FIG. 29).

These results suggest for the first time that for a gene editing project where a single point mutation (C->T) is desired, the C11 architecture is the best suited for such focus, especially with respect to target sequences displaying a 15 bp spacer. Such target sequence may be defined by the general formula:

5′-T₀-N_{left}-N_{y}-RTC-Nₓ-N_{right}-A₀-3′;
or
5′-T₀-N_{left}-Nₓ-GAY-N_{y}-N_{right}-A₀-3′

wherein
N can be A, T, C or G
R can be G or A, preferentially G,
Y can be C or T
N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
   and preferably by the formula:

5′-T₀-N_{left}-N_{y}-RTCC-Nₓ-N_{right}-A₀-3′;
or
5′-T₀-N_{left}-Nₓ-GGAY-N_{y}-N_{right}-A₀-3′

more preferably by the formula

5′-T₀-N_{left}-N_{y}-GTCC-Nₓ-N_{right}-A₀-3′;
or
5′-T₀-N_{left}-Nₓ-GGAC-N_{y}-N_{right}-A₀-3′

wherein
x=2 to 6
y=6 to 10
with x+y=11.

### Influence of the Context Around TC: 13 bp Spacer Length.

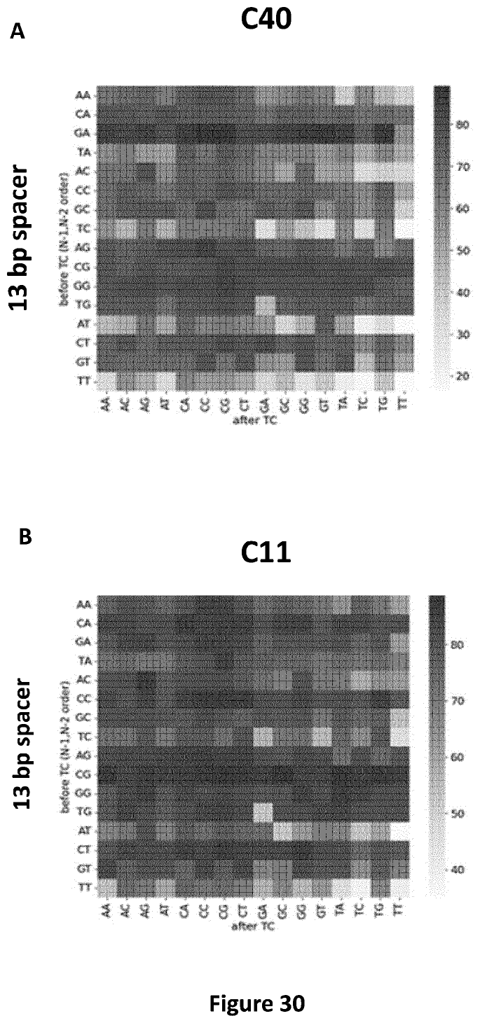

For the 13 bp spacer length, other libraries comprising 256 unique ssODN were designed (as detailed in Table 17). PBMCs from 2 donors were transfected with TRAC TALEN and the three different pools of oligos to be inserted in the TRAC locus, followed by either STAT3 BE C40/C40 or C11/C11 transfection for editing of the cells with oligo KI. gDNA was made from cells treated with the 8 oligo pools, and samples were sent for sequencing on MiSeq. Data analysis from bioinformatics represented in FIGS. 30 A and B) determined the contribution of each surrounding base to the efficiency of C editing, which was found to be similar for both architectures.
at position M2: T<A<<G=C. This position seems to be important while not contiguous to the TC.
at position M1: T<C<A<G
at position 1: T<G<A<C
at position 2: T<A=C<G. This position seems to be the less important one, as with C11 on the same spacer.

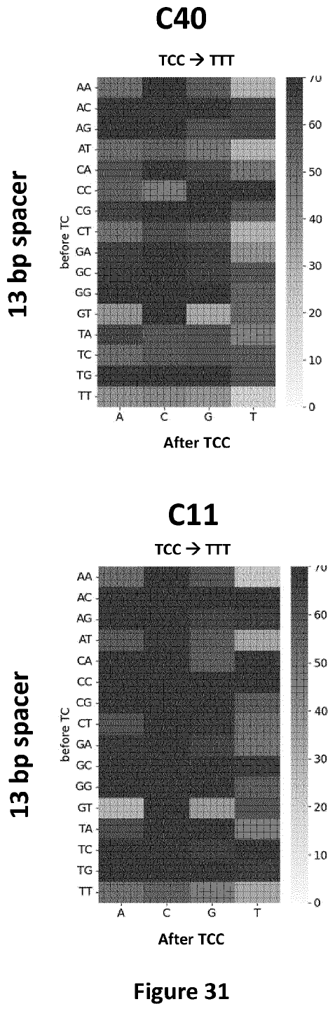

We also looked at multiple editions where Cs do follow the central TC (TCC to TTT) and editing analysis showed that the edition of both Ts on the 13 bp is more permissive for both architectures (FIG. 31).

TALEB looks surprisingly more permissive when targeting sequences displaying 13 bp spacers than with target sequences displaying 15 bp spacers. These results suggest that when multiple edits are desired for a gene editing project, the design of TALE base editors should be preferably designed with respect to genomic sequences displaying a 13 bp spacer. Such target sequence may be defined by the general formula:

5′-T₀-N_{left}-N_{y}-RTC-Nₓ-N_{right}-A₀-3′;
or
5′-T₀-N_{left}-Nₓ-GAY-N_{y}-N_{right}-A₀-3′

wherein N can be A, T, C or G R can be G or A.
Y can be C or T
N_{left} can be a polynucleotide sequence comprising between 9 to 20 nucleotides, where each individual nucleotide can be A, T, C or G;
N_{right} can be a polynucleotide sequence comprising between 9 to 20 nucleotides, where each individual nucleotide can be A, T, C or G;
G being the complementary base of C.
   and preferably by the formula:

5′-T₀-N_{left}-N_{y}-RTCC-Nₓ-N_{right}-A₀-3′;
or
5′-T₀-N_{left}-Nₓ-GGAY-N_{y}-N_{right}-A₀-3′

and more preferably

5′-T₀-N_{left}-N_{y}-GTCC-Nₓ-N_{right}-A₀-3′;
or
5′-T₀-N_{left}-Nₓ-GGAC-N_{y}-N_{right}-A₀-3′

wherein:
x=2 to 4
y=6 to 8
with x+y=9

As represented in FIGS. 28 (A and B), data analysis from bioinformatics aiming at determining the contribution of each surrounding base to the efficiency of C editing surprisingly showed quite similar results in terms of the bases surrounding TC for determining high editing targets comprising the different spacers. However, irrespective of the spacers, the C11 TALEB scaffold displayed a stronger specificity on those target sequences.

### Materials and Methods

### T Cell Culture

Cryopreserved human PBMCs were acquired from ALLCELLS. PBMCs were cultured in X-vivo-15 media (Lonza Group), containing 20 ng/ml human IL-2 (Miltenyi Biotec), and 5% human serum AB (Seralab). Human T cell activator TransAct (Miltenyi Biotec) was used to activate T cells at 25 μl TransAct per million CD3+ cells the day after thawing the PBMCs. TransAct was kept in the culture media for 72 hours.

### TALE-Nuclease or TALE-Base Editors Production

TALEN (pCLS32783) and TALE-base editors (pCLS35714, pCLS35715, pCLS37473 and pCLS37474, Table 13) backbones were assembled using standard molecular biology and/or microbiology technics such as enzymatic restriction digestion, ligation, bacterial transformation and plasmid DNA extraction. TALE DNA targeting array were assembled and cloned in TALEN and/or TALE-base editors backbones using standard molecular biology and/or microbiology technics such as enzymatic restriction digestion, ligation, bacterial transformation (NEB 10-beta competent E. coli for ccdB selection or NEB stable competent E. coli for blue/white screening) and plasmid DNA extraction.

Large Scale TALE-Nuclease and TALE-Base Editors mRNA Production

### (STA T3 Targeting TALEB)

Plasmids encoding the TRAC TALE-Nuclease contained a T7 promoter and a polyA sequence. The TALE-Nuclease mRNA from the TRAC TALE-Nuclease plasmid was produced by Trilink. Sequence targeted by the TRAC TALE-Nuclease (17-bp recognition sites, upper case letters, separated by a 15-bp spacer).

Plasmids encoding STAT3 TALE base editors contained a T7 promoter and a polyA sequence. Sequence verified plasmids were linearized with SapI (NEB) before in vitro mRNA synthesis. mRNA was produced with NEB HiScribe™ T7 Quick High Yield RNA Synthesis Kit (NEB). The 5′capping reaction was performed with ScriptCap™ m7G Capping System (Cellscript). Antarctic Phosphatase (NEB) was used to treat the capped mRNA and the final cleanups was performed with Mag-Bind TotalPure NGS beads (Omega bio-tek) and Invitrogen DynaMag-2 Magnet (ThermoFisher).

ssODN Repair Template Transfection

The ssODN pool targeting the TRAC locus (Table 15, Table 16 and Table 17) were ordered from Integrated DNA Technologies (IDT) and resuspended in ddH2O at 50 pmol/μl.

T cells activated with TransACT for 3 days were transferred into fresh complete media containing 20 ng/ml human IL-2 (Miltenyi Biotec), and 5% human serum AB (Seralab) 10-12 hrs before transfection.

The harvested cells were washed once with warm PBS. 1E6 PBS washed cells were pelleted and resuspended in 20 μl Lonza P3 primary cell buffer (Lonza). 200 pmol ssODN pool and 1 mg/arm of TRAC TALE-Nuclease were mixed with the cell and then the cell mixture was electroporated using the Lonza 4D-Nucleofector under the E0115 program for stimulated human T cells. After electroporation, 80 μl warm complete media was added to the cuvette to dilute the electroporation buffer, the mixture was then carefully transferred to 400 ml pre-warmed complete media in 48-well plates. Cells transfected with ssODN and TALE-Nuclease were then incubated at 30° C. until 24 hrs post TALE-Nuclease transfection before transfer back to 37° C.

Cells with ssODN KI were cultured for two days before harvesting for TALEB treatment. The harvested cells were washed once with warm PBS. 1E6 PBS washed cells were pelleted and resuspended in 20 μl Lonza P3 primary cell buffer (Lonza). 1 mg/arm of STAT3 TALEB (CO, C11 or C40) were mixed with the cell and then the cell mixture was electroporated using the Lonza 4D-Nucleofector under the E0115 program for stimulated human T cells. After electroporation, 80 μl warm complete media was added to the cuvette to dilute the electroporation buffer, the mixture was then carefully transferred to 400 ml pre-warmed complete media in 48-well plates. Cells transfected with TALE base editors incubated at 37° C. for 2 more days before harvesting for gDNA extraction and NGS analysis.

### Genomic DNA Extraction

Cells were harvested and washed once with PBS. Genomic DNA extraction was performed using Mag-Bind Blood & Tissue DNA HDQ kits (Omega Bio-Tek) following the manufacturer's instructions.

### Targeted PCR and NGS

100 mg genomic DNA was used per reaction in a 50 ml reaction with Phusion High-Fidelity PCR Master Mix (NEB). The PCR condition was set to 1 cycle of 30 s at 98° C.; 30 cycles of 10 s at 98° C., 30 s at 60° C., 30 s at 72° C.; 1 cycle of 5 min at 72° C.; hold at 4° C. The PCR product was then purified with Omega NGS beads (1:1.2 ratio) and eluted into 30 ml of 10 mM Tris buffer pH7.4. The second PCR which incorporates NGS indices was then performed on the purified product from the first PCR. 15 ul of the first PCR product were set in a 50 ml reaction with Phusion High-Fidelity PCR Master Mix (NEB). The PCR condition was set to 1 cycle of 30 s at 98° C.; 8 cycles of 10 s at 98° C., 30 s at 62° C., 30 s at 72° C.; 1 cycle of 5 min at 72° C.; hold at 4° C. Purified PCR products were sequenced on MiSeq (Illumina) on a 2×250 nano V2 cartridge.

### Example 7: TALEB According to the Invention Prevent from AAV Trapping

At day 0, frozen human Peripheral Blood Mononuclear Cells (PBMC) from AllCells (Alameda, California 94502) were thawed, washed, counted and resuspended in OpTmizer medium (Gibco: A1048501) supplemented with 5% AB serum (GeminiBio: 100-318) and 20 ng/mL recombinant human IL-2 (Miltenyi: 130-097-743). The cells were then transferred to an incubator set at 37° C., 5% C02.

At day 1, PBMC were counted, analysed by flow cytometry to assess the % of CD3+ cells, centrifuged and resuspended in Optimizer medium supplemented with 5% AB serum, 20 ng/mL human IL-2 and Transact beads CD3 CD28 (Miltenyi: 130-111-160). The cells were then transferred to an incubator set at 37° C., 5% C02.

At day 4, T-cells were sub-cultured into fresh OpTimizer medium-supplemented with 5% AB serum, 20 ng/ml IL-2. The plates were then transferred to an incubator set at 37° C., 5% C02.

At day 5, cells were co-electroporated using the AgilePulse technology with 1 μg of mRNA encoding the left and right arms of either TRAC TALEN (SEQ ID NO: 562 and 563) or B2M TALEN (SEQ ID NO: 564 and 565) or TRAC TALEB (SEQ ID NO: 566 and 567) targeting the TRAC genomic sequence SEQ ID NO:561. Upon transfection, cells were incubated in fresh OpTimizer medium for 15 min at 37° C. and then transferred to 30° C. for an additional 15 minutes. Cells were then counted, concentrated to 8E6 cells/mL and transduced or not with 50000 vg/cell of AAV6 particles encoding HLA-E (SEQ ID NO:560) for targeted integration at the B2M locus (SEQ ID NO: 559) as previously reported [Jo et al (2022) Nat Commun 13(1) and Sachdeva et al. (2019) Nat Commun. 10 (1)]. Modified cells were cultured overnight at 30° C. and next day they were sub-cultured into fresh OpTimizer medium-supplemented with 5% AB serum, 20 ng/ml IL-2. Cells were then transferred to an incubator set at 37° C., 5% C02.

At day 8, modified T cells were harvested and analysed by flow cytometry with anti-TCRab, anti-HLA-ABC and anti-HLA-E antibodies.

The sequences of the reagents used in these experiments are reported in Table 18.

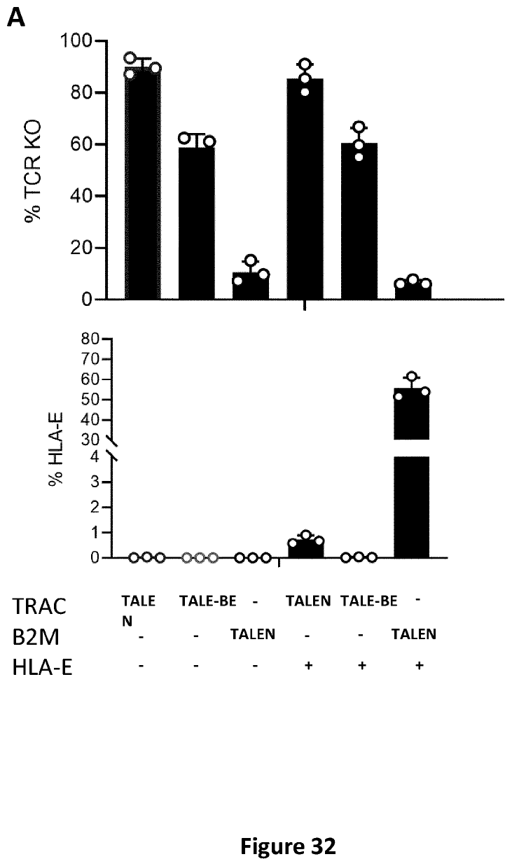

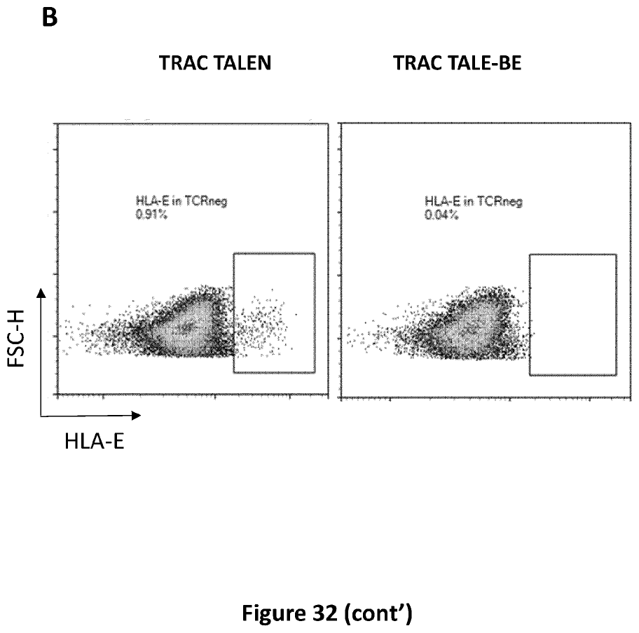

As shown in FIG. 32A, approximately 80%, 60% and 10% of cells were TCRab negative, when treated with TRAC TALEN, TRAC TALEB and B2M TALEN respectively.

This result demonstrates that TRAC TALEN and TALE base editors were both highly effective. In addition, when transduced with AAV6 particles, 50% of HLA-E positive cells could be detected when cells were transfected with B2M TALEN demonstrating high targeting efficiency. When cells were transfected with TRAC TALEN and transduced with AAV6 particles (used as template DNA designed for insertion of the HLAE coding sequence at the B2M locus by homologous recombination) around 0.5% of HLA-E positive cells could be detected. These HLA-E positive cells were not artefact as shown in FIG. 32B and these results demonstrate that AAV6 construct could be trapped at the TRAC locus, although the DNA template was not primarily designed to be inserted at the TRAC locus. Importantly no HLA-E positive cells could be detected when cells were transfected with TRAC TALE base editors and transduced with AAV6 particles (FIGS. 32A and 32B) demonstrating that such trapping can be abolished when using a TALE base editors. Thus, the combination of TALEN and TALEB was found to be highly efficient and prompt to ensure higher genome integrity when performing multiple gene edits in therapeutic immune cells, especially when combining gene edits consisting of knocking in a transgene using a TALE nuclease and knocking-out an endogenous gene by using a TALEB as per the present invention.

TABLE 18
polynucleotide and polypeptides used in Example 7

| Polynucleotide | SEQ | |
|---|---|---|
| designation | ID # | Polynucleotide sequences |
| Target | 559 | TTAGCTGTGCTCGCGCTactctctctttctGGCCTGGAGGCTATCCA |
| sequence | | |
| integration | | |
| B2M | | |
| HLAE AAV | 560 | CGCACCCCAGATCGGAGGGCGCCGATGTACAGACAGCAAACTCACCCAGTCTAGTGCA |
| sequence | | TGCCTTCTTAAACATCACGAGACTCTAAGAAAAGGAAACTGAAAACGGGAAAGTCCCT |
| | | CTCTCTAACCTGGCACTGCGTCGCTGGCTTGGAGACAGGTGACGGTCCCTGCGGGCCT |
| | | TGTCCTGATTGGCTGGGCACGCGTTTAATATAAGTGGAGGCGTCGCGCTGGCGGGCAT |
| | | TCCTGAAGCTGACAGCATTCGGGCCGAGATGTCTCGCTCCGTGGCCTTAGCTGTGCTCG |
| | | CGCTACTCTCTCTTAGCGGCCTCGAAGCTGTTATGGCTCCGCGGACTTTAATTTTAGGT |
| | | GGTGGCGGATCCGGTGGTGGCGGTTCTGGTGGTGGCGGCTCCATCCAGCGTACGCCC |
| | | AAAATTCAAGTCTACAGCCGACATCCTGCAGAGAACGGCAAATCTAATTTCCTGAACTG |
| | | CTATGTATCAGGCTTTCACCCTAGCGATATAGAAGTGGACCTGCTGAAAAACGGAGAG |
| | | AGGATAGAAAAGGTCGAACACAGCGACCTCTCCTTTTCCAAGGACTGGAGCTTTTATCT |
| | | TCTGTATTATACTGAATTTACACCCACGGAAAAAGATGAGTATGCGTGCCGAGTAAACC |
| | | ACGTCACGCTGTCACAGCCCAAAATAGTAAAATGGGATCGCGACATGGGTGGTGGCG |
| | | GTTCTGGTGGTGGCGGTAGTGGCGGCGGAGGAAGCGGTGGTGGCGGTTCCGGATCTC |
| | | ACTCCTTGAAGTATTTCCACACTTCCGTGTCCCGGCCCGGCCGCGGGGAGCCCCGCTTC |
| | | ATCTCTGTGGGCTACGTGGACGACACCCAGTTCGTGCGCTTCGACAACGACGCCGCGA |
| | | GTCCGAGGATGGTGCCGCGGGCGCCGTGGATGGAGCAGGAGGGGTCAGAGTATTGG |
| | | GACCGGGAGACACGGAGCGCCAGGGACACCGCACAGATTTTCCGAGTGAACCTGCGG |
| | | ACGCTGCGCGGCTACTACAATCAGAGCGAGGCCGGGTCTCACACCCTGCAGTGGATGC |
| | | ATGGCTGCGAGCTGGGGCCCGACAGGCGCTTCCTCCGCGGGTATGAACAGTTCGCCTA |
| | | CGACGGCAAGGATTATCTCACCCTGAATGAGGACCTGCGCTCCTGGACCGCGGTGGAC |
| | | ACGGCGGCTCAGATCTCCGAGCAAAAGTCAAATGATGCCTCTGAGGCGGAGCACCAG |
| | | AGAGCCTACCTGGAAGACACATGCGTGGAGTGGCTCCACAAATACCTGGAGAAGGGG |
| | | AAGGAGACGCTGCTTCACCTGGAGCCCCCAAAGACACACGTGACTCACCACCCCATCTC |
| | | TGACCATGAGGCCACCCTGAGGTGCTGGGCTCTGGGCTTCTACCCTGCGGAGATCACA |
| | | CTGACCTGGCAGCAGGATGGGGAGGGCCATACCCAGGACACGGAGCTCGTGGAGACC |
| | | AGGCCTGCAGGGGATGGAACCTTCCAGAAGTGGGCAGCTGTGGTGGTGCCTTCTGGA |
| | | GAGGAGCAGAGATACACGTGCCATGTGCAGCATGAGGGGCTACCCGAGCCCGTCACC |
| | | CTGAGATGGAAGCCGGCTTCCCAGCCCACCATCCCCATCGTGGGCATCATTGCTGGCCT |
| | | GGTTCTCCTTGGATCTGTGGTCTCTGGAGCTGTGGTTGCTGCTGTGATATGGAGGAAG |
| | | AAGAGCTCAGGTGGAAAAGGAGGGAGCTACTATAAGGCTGAGTGGAGCGACAGTGC |
| | | CCAGGGGTCTGAGTCTCACAGCTTGTAACTGTGCCTTCTAGTTGCCAGCCATCTGTTGT |
| | | TTGCCCCTCCCCCGTGCCTTCCTTGACCCTGGAAGGTGCCACTCCCACTGTCCTTTCCTA |
| | | ATAAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTG |
| | | GGGATGCGGTGGGCTCTATGTCTCTTTCTGGCCTGGAGGCTATCCAGCGTGAGTCTCTC |
| | | CTACCCTCCCGCTCTGGTCCTTCCTCTCCCGCTCTGCACCCTCTGTGGCCCTCGCTGTGCT |
| | | CTCTCGCTCCGTGACTTCCCTTCTCCAAGTTCTCCTTGGTGGCCCGCCGTGGGGCTAGTC |
| | | CAGGGCTGGATCTCGGGGAAGCGGCGGGGTGGCCTGGGAGTGGGGAAGGGGGTGC |
| | | GCACCCGGGACGCGCGCTACTTGCCCCTTTCGGCGGGGAGCAGGGGAGACCTTTGGC |
| | | CTACGGCGACGGGAGGGTCGGGAC |
| TRAC TALEN | 561 | TGATCCTCTTGTCCCACAGATATCCagaaccctgaccctgCCGTGTACCAGCTGAGAGA |
| inactivation | | |
| target | | |
| sequence | | |
| Polypeptide | SEQ | |
| designation | ID # | Polynucleotide sequences |
| TRAC TALEN | 562 | MGDPKKKRKVIDIADLRTLGYSQQQQEKIKPKVRSTVAQHHEALVGHGFTHAHI |
| Left | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPQQVVAIASNG |
| | | GGKQALETVQRLLPVLCQAHGLTPQQVVAIASNNGGKQALETVQRLLPVLCQAH |
| | | GLTPQQVVAIASNGGGKQALETVQRLLPVLCQAHGLTPEQVVAIASHDGGKQAL |
| | | ETVQRLLPVLCQAHGLTPEQVVAIASHDGGKQALETVQRLLPVLCQAHGLTPEQ |
| | | VVAIASHDGGKQALETVQRLLPVLCQAHGLTPEQVVAIASNIGGKQALETVQAL |
| | | LPVLCQAHGLTPEQVVAIASHDGGKQALETVQRLLPVLCQAHGLTPEQVVAIAS |
| | | NIGGKQALETVQALLPVLCQAHGLTPQQVVAIASNNGGKQALETVQRLLPVLCQ |
| | | AHGLTPEQVVAIASNIGGKQALETVQALLPVLCQAHGLTPQQVVAIASNGGGKQ |
| | | ALETVQRLLPVLCQAHGLTPEQVVAIASNIGGKQALETVQALLPVLCQAHGLTP |
| | | QQVVAIASNGGGKQALETVQRLLPVLCQAHGLTPEQVVAIASHDGGKQALETVQ |
| | | RLLPVLCQAHGLTPQQVVAIASNGGGRPALESIVAQLSRPDPALAALTNDHLVA |
| | | LACLGGRPALDAVKKGLGDPISRSQLVKSELEEKKSELRHKLKYVPHEYIELIE |
| | | IARNSTQDRILEMKVMEFFMKVYGYRGKHLGGSRKPDGAIYTVGSPIDYGVIVD |
| | | TKAYSGGYNLPIGQADEMQRYVEENQTRNKHINPNEWWKVYPSSVTEFKFLFVS |
| | | GHFKGNYKAQLTRLNHITNCNGAVLSVEELLIGGEMIKAGTLTLEEVRRKFNNG |
| | | EINFAAD |
| TRAC TALEN | 563 | MGDPKKKRKVIDIADLRTLGYSQQQQEKIKPKVRSTVAQHHEALVGHGFTHAHI |
| Right | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPEQVVAIASHD |
| | | GGKQALETVQRLLPVLCQAHGLTPQQVVAIASNGGGKQALETVQRLLPVLCQAH |
| | | GLTPEQVVAIASHDGGKQALETVQRLLPVLCQAHGLTPEQVVAIASNIGGKQAL |
| | | ETVQALLPVLCQAHGLTPQQVVAIASNNGGKQALETVQRLLPVLCQAHGLTPEQ |
| | | VVAIASHDGGKQALETVQRLLPVLCQAHGLTPQQVVAIASNGGGKQALETVQRL |
| | | LPVLCQAHGLTPQQVVAIASNNGGKQALETVQRLLPVLCQAHGLTPQQVVAIAS |
| | | NNGGKQALETVQRLLPVLCQAHGLTPQQVVAIASNGGGKQALETVQRLLPVLCQ |
| | | AHGLTPEQVVAIASNIGGKQALETVQALLPVLCQAHGLTPEQVVAIASHDGGKQ |
| | | ALETVQRLLPVLCQAHGLTPEQVVAIASNIGGKQALETVQALLPVLCQAHGLTP |
| | | EQVVAIASHDGGKQALETVQRLLPVLCQAHGLTPQQVVAIASNNGGKQALETVQ |
| | | RLLPVLCQAHGLTPQQVVAIASNGGGRPALESIVAQLSRPDPALAALTNDHLVA |
| | | LACLGGRPALDAVKKGLGDPISRSQLVKSELEEKKSELRHKLKYVPHEYIELIE |
| | | IARNSTQDRILEMKVMEFFMKVYGYRGKHLGGSRKPDGAIYTVGSPIDYGVIVD |
| | | TKAYSGGYNLPIGQADEMQRYVEENQTRNKHINPNEWWKVYPSSVTEFKFLFVS |
| | | GHFKGNYKAQLTRLNHITNCNGAVLSVEELLIGGEMIKAGTLTLEEVRRKFNNG |
| | | EINFAAD |
| B2M TALEN | 564 | MGDPKKKRKVIDIADLRTLGYSQQQQEKIKPKVRSTVAQHHEALVGHGFTHAHI |
| Left | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPQQVVAIASNG |
| | | GGKQALETVQRLLPVLCQAHGLTPQQVVAIASNNGGKQALETVQRLLPVLCQAH |
| | | GLTPQQVVAIASNGGGKQALETVQRLLPVLCQAHGLTPEQVVAIASHDGGKQAL |
| | | ETVQRLLPVLCQAHGLTPEQVVAIASHDGGKQALETVQRLLPVLCQAHGLTPEQ |
| | | VVAIASHDGGKQALETVQRLLPVLCQAHGLTPEQVVAIASNIGGKQALETVQAL |
| | | LPVLCQAHGLTPEQVVAIASHDGGKQALETVQRLLPVLCQAHGLTPEQVVAIAS |
| | | NIGGKQALETVQALLPVLCQAHGLTPQQVVAIASNNGGKQALETVQRLLPVLCQ |
| | | AHGLTPEQVVAIASNIGGKQALETVQALLPVLCQAHGLTPQQVVAIASNGGGKQ |
| | | ALETVQRLLPVLCQAHGLTPEQVVAIASNIGGKQALETVQALLPVLCQAHGLTP |
| | | QQVVAIASNGGGKQALETVQRLLPVLCQAHGLTPEQVVAIASHDGGKQALETVQ |
| | | RLLPVLCQAHGLTPQQVVAIASNGGGRPALESIVAQLSRPDPALAALTNDHLVA |
| | | LACLGGRPALDAVKKGLGDPISRSQLVKSELEEKKSELRHKLKYVPHEYIELIE |
| | | IARNSTQDRILEMKVMEFFMKVYGYRGKHLGGSRKPDGAIYTVGSPIDYGVIVD |
| | | TKAYSGGYNLPIGQADEMQRYVEENQTRNKHINPNEWWKVYPSSVTEFKFLFVS |
| | | GHFKGNYKAQLTRLNHITNCNGAVLSVEELLIGGEMIKAGTLTLEEVRRKFNNG |
| | | EINFAAD |
| B2M TALEN | 565 | MGDPKKKRKVIDIADLRTLGYSQQQQEKIKPKVRSTVAQHHEALVGHGFTHAHI |
| Right | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPQQVVAIASNN |
| | | GGKQALETVQRLLPVLCQAHGLTPQQVVAIASNNGGKQALETVQRLLPVLCQAH |
| | | GLTPEQVVAIASNIGGKQALETVQALLPVLCQAHGLTPQQVVAIASNGGGKQAL |
| | | ETVQRLLPVLCQAHGLTPEQVVAIASNIGGKQALETVQALLPVLCQAHGLTPQQ |
| | | VVAIASNNGGKQALETVQRLLPVLCQAHGLTPEQVVAIASHDGGKQALETVQRL |
| | | LPVLCQAHGLTPEQVVAIASHDGGKQALETVQRLLPVLCQAHGLTPQQVVAIAS |
| | | NGGGKQALETVQRLLPVLCQAHGLTPEQVVAIASHDGGKQALETVQRLLPVLCQ |
| | | AHGLTPEQVVAIASHDGGKQALETVQRLLPVLCQAHGLTPEQVVAIASNIGGKQ |
| | | ALETVQALLPVLCQAHGLTPQQVVAIASNNGGKQALETVQRLLPVLCQAHGLTP |
| | | QQVVAIASNNGGKQALETVQRLLPVLCQAHGLTPEQVVAIASHDGGKQALETVQ |
| | | RLLPVLCQAHGLTPQQVVAIASNGGGRPALESIVAQLSRPDPALAALTNDHLVA |
| | | LACLGGRPALDAVKKGLGDPISRSQLVKSELEEKKSELRHKLKYVPHEYIELIE |
| | | IARNSTQDRILEMKVMEFFMKVYGYRGKHLGGSRKPDGAIYTVGSPIDYGVIVD |
| | | TKAYSGGYNLPIGQADEMQRYVEENQTRNKHINPNEWWKVYPSSVTEFKFLFVS |
| | | GHFKGNYKAQLTRLNHITNCNGAVLSVEELLIGGEMIKAGTLTLEEVRRKFNNG |
| | | EINFAAD |
| TRAC TALEB | 566 | MGDPKKKRKVIDIADLRTLGYSQQQQEKIKPKVRSTVAQHHEALVGHGFTHAHI |
| Left | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPQQVVAIASNN |
| | | GGKQALETVQRLLPVLCQAHGLTPQQVVAIASNGGGKQALETVQRLLPVLCQAH |
| | | GLTPQQVVAIASNNGGKQALETVQRLLPVLCQAHGLTPEQVVAIASNIGGKQAL |
| | | ETVQALLPVLCQAHGLTPEQVVAIASHDGGKQALETVQRLLPVLCQAHGLTPEQ |
| | | VVAIASNIGGKQALETVQALLPVLCQAHGLTPEQVVAIASHDGGKQALETVQRL |
| | | LPVLCQAHGLTPEQVVAIASNIGGKQALETVQALLPVLCQAHGLTPQQVVAIAS |
| | | NGGGKQALETVQRLLPVLCQAHGLTPQQVVAIASNGGGKQALETVQRLLPVLCQ |
| | | AHGLTPQQVVAIASNGGGRPALESIVAQLSRPDPALAALTNDHLVALACLGGRP |
| | | ALDAVKKGLGGSAIPVKRGATGETKVFTGNSNSPKSPTKGGCSGGSTNLSDIIE |
| | | KETGKQLVIQESILMLPEEVEEVIGNKPESDILVHTAYDESTDENVMLLTSDAP |
| | | EYKPWALVIQDSNGENKIKM |
| TRAC TALEB | 567 | MGDPKKKRKVIDIADLRTLGYSQQQQEKIKPKVRSTVAQHHEALVGHGFTHAHI |
| Right | | VALSQHPAALGTVAVKYQDMIAALPEATHEAIVGVGKQWSGARALEALLTVAGE |
| | | LRGPPLQLDTGQLLKIAKRGGVTAVEAVHAWRNALTGAPLNLTPQQVVAIASNN |
| | | GGKQALETVQRLLPVLCQAHGLTPEQVVAIASHDGGKQALETVQRLLPVLCQAH |
| | | GLTPEQVVAIASHDGGKQALETVQRLLPVLCQAHGLTPQQVVAIASNGGGKQAL |
| | | ETVQRLLPVLCQAHGLTPEQVVAIASNIGGKQALETVQALLPVLCQAHGLTPQQ |
| | | VVAIASNGGGKQALETVQRLLPVLCQAHGLTPQQVVAIASNGGGKQALETVQRL |
| | | LPVLCQAHGLTPEQVVAIASHDGGKQALETVQRLLPVLCQAHGLTPEQVVAIAS |
| | | NIGGKQALETVQALLPVLCQAHGLTPEQVVAIASHDGGKQALETVQRLLPVLCQ |
| | | AHGLTPEQVVAIASHDGGKQALETVQRLLPVLCQAHGLTPQQVVAIASNNGGKQ |
| | | ALETVQRLLPVLCQAHGLTPEQVVAIASNIGGKQALETVQALLPVLCQAHGLTP |
| | | QQVVAIASNGGGKQALETVQRLLPVLCQAHGLTPQQVVAIASNGGGRPALESIV |
| | | AQLSRPDPALAALTNDHLVALACLGGRPALDAVKKGLGGSGSYALGPYQISAPQ |
| | | LPAYNGQTVGTFYYVNDAGGLESKVFSSGGPTPYPNYANAGHVEGQSALFMRDN |
| | | GISEGLVFHNNPEGTCGFCVNMTETLLPENAKMTVVPPEGSGGSTNLSDIIEKE |
| | | TGKQLVIQESILMLPEEVEEVIGNKPESDILVHTAYDESTDENVMLLTSDAPEY |
| | | KPWALVIQDSNGENKIKML |

TABLE 7
List of TALEB target sequence windows following the rules of the present invention
to introduce mutations in the TRAC gene.

| | | | | Impact at | |
|---|---|---|---|---|---|
| | | | | trans- | |
| | | SEQ | | criptional/ | |
| Sequence | | ID | | trans- | |
| designation | Target window sequence in TRAC centered | NO | Base | lational | Protein |
| (TRAC) | on TC/AG to be mutated (62 bp) | # | −1 | level | domain |
| TRAC Cluster 0 | ATGTCCTAACCCTGATCCTCTTGTCCCACAGATATCCAGAACCCTGACC | 366 | A | Splice site | |
| | CTGCCGTGTACCA | | | | |
| TRAC Cluster 1 | CCTAACCCTGATCCTCTTGTCCCACAGATATCCAGAACCCTGACCCTGC | 367 | A | Q -> stop | |
| | CGTGTACCAGCTG | | | | |
| TRAC Cluster 2 | TCCTCTTGTCCCACAGATATCCAGAACCCTGACCCTGCCGTGTACCAGC | 368 | G | D -> N | |
| | TGAGAGACTCTAA | | | | |
| TRAC Cluster 3 | AGAACCCTGACCCTGCCGTGTACCAGCTGAGAGACTCTAAATCCAGTGA | 369 | C | R -> K | |
| | CAAGTCTGTCTGC | | | | |
| TRAC Cluster 4 | AACCCTGACCCTGCCGTGTACCAGCTGAGAGACTCTAAATCCAGTGACA | 370 | G | D -> N | |
| | AGTCTGTCTGCCT | | | | |
| TRAC Cluster 5 | CCTGACCCTGCCGTGTACCAGCTGAGAGACTCTAAATCCAGTGACAAGT | 371 | C | S -> F | |
| | CTGTCTGCCTATT | | | | |
| TRAC Cluster 6 | CCTGCCGTGTACCAGCTGAGAGACTCTAAATCCAGTGACAAGTCTGTCT | 372 | A | S -> F | |
| | GCCTATTCACCGA | | | | |
| TRAC Cluster 7 | GTGTACCAGCTGAGAGACTCTAAATCCAGTGACAAGTCTGTCTGCCTAT | 373 | G | D -> N | |
| | TCACCGATTTTGA | | | | |
| TRAC Cluster 8 | CAGCTGAGAGACTCTAAATCCAGTGACAAGTCTGTCTGCCTATTCACCG | 374 | G | S -> F | |
| | ATTTTGATTCTCA | | | | |
| TRAC Cluster 9 | TCCAGTGACAAGTCTGTCTGCCTATTCACCGATTTTGATTCTCAAACAA | 375 | A | D -> N | |
| | ATGTGTCACAAAG | | | | |
| TRAC Cluster 10 | GACAAGTCTGTCTGCCTATTCACCGATTTTGATTCTCAAACAAATGTGT | 376 | A | D -> N | |
| | CACAAAGTAAGGA | | | | |
| TRAC Cluster 11 | AAGTCTGTCTGCCTATTCACCGATTTTGATTCTCAAACAAATGTGTCAC | 377 | T | S -> F | |
| | AAAGTAAGGATTC | | | | |
| TRAC Cluster 12 | GTCTGTCTGCCTATTCACCGATTTTGATTCTCAAACAAATGTGTCACAA | 378 | C | Q -> stop | |
| | AGTAAGGATTCTG | | | | |
| TRAC Cluster 13 | TTCACCGATTTTGATTCTCAAACAAATGTGTCACAAAGTAAGGATTCTG | 379 | G | S -> L | |
| | ATGTGTATATCAC | | | | |
| TRAC Cluster 14 | GATTCTCAAACAAATGTGTCACAAAGTAAGGATTCTGATGTGTATATCA | 380 | A | D -> N | |
| | CAGACAAAACTGT | | | | |
| TRAC Cluster 15 | TCTCAAACAAATGTGTCACAAAGTAAGGATTCTGATGTGTATATCACAG | 381 | T | S -> F | |
| | ACAAAACTGTGCT | | | | |
| TRAC Cluster 16 | CAAACAAATGTGTCACAAAGTAAGGATTCTGATGTGTATATCACAGACA | 382 | A | D -> N | |
| | AAACTGTGCTAGA | | | | |
| TRAC Cluster 17 | CAAAGTAAGGATTCTGATGTGTATATCACAGACAAAACTGTGCTAGACA | 383 | G | D -> N | |
| | TGAGGTCTATGGA | | | | |
| TRAC Cluster 18 | GATGTGTATATCACAGACAAAACTGTGCTAGACATGAGGTCTATGGACT | 384 | G | D -> N | |
| | TCAAGAGCAACAG | | | | |
| TRAC Cluster 19 | GTATATCACAGACAAAACTGTGCTAGACATGAGGTCTATGGACTTCAAG | 385 | C | M -> I | |
| | AGCAACAGTGCTG | | | | |
| TRAC Cluster 20 | ATCACAGACAAAACTGTGCTAGACATGAGGTCTATGGACTTCAAGAGCA | 386 | G | S -> F | |
| | ACAGTGCTGTGGC | | | | |
| TRAC Cluster 21 | GACAAAACTGTGCTAGACATGAGGTCTATGGACTTCAAGAGCAACAGTG | 387 | G | D -> N | |
| | CTGTGGCCTGGAG | | | | |
| TRAC Cluster 22 | GGACTTCAAGAGCAACAGTGCTGTGGCCTGGAGCAACAAATCTGACTTT | 388 | C | W -> stop | |
| | GCATGTGCAAACG | | | | |
| TRAC Cluster 23 | AGCAACAGTGCTGTGGCCTGGAGCAACAAATCTGACTTTGCATGTGCAA | 389 | A | S -> F | |
| | ACGCCTTCAACAA | | | | |
| TRAC Cluster 24 | AACAGTGCTGTGGCCTGGAGCAACAAATCTGACTTTGCATGTGCAAACG | 390 | G | D -> N | |
| | CCTTCAACAACAG | | | | |
| TRAC Cluster 25 | GCAAACGCCTTCAACAACAGCATTATTCCAGAAGACACCTTCTTCCCCA | 391 | T | E -> K | |
| | GCCCAGGTAAGGG | | | | |
| TRAC Cluster 26 | AACGCCTTCAACAACAGCATTATTCCAGAAGACACCTTCTTCCCCAGCC | 392 | G | D -> N | |
| | CAGGTAAGGGCAG | | | | |
| TRAC Cluster 27 | ACAACAGCATTATTCCAGAAGACACCTTCTTCCCCAGCCCAGGTAAGGG | 393 | T | P -> S | |
| | CAGCTTTGGTGCC | | | | |
| TRAC Cluster 28 | ATGCTGAAAGAATGTCTGTTTTTCCTTTTAGAAAGTTCCTGTGATGTCA | 394 | T | Splice site | |
| | AGCTGGTCGAGAA | | | | |
| TRAC Cluster 29 | AAAGAATGTCTGTTTTTCCTTTTAGAAAGTTCCTGTGATGTCAAGCTGG | 395 | T | S -> F | |
| | TCGAGAAAAGCTT | | | | |
| TRAC Cluster 30 | TGTCTGTTTTTCCTTTTAGAAAGTTCCTGTGATGTCAAGCTGGTCGAGA | 396 | A | D -> N | |
| | AAAGCTTTGAAAC | | | | |
| TRAC Cluster 31 | TTAGAAAGTTCCTGTGATGTCAAGCTGGTCGAGAAAAGCTTTGAAACAG | 397 | C | E -> K | |
| | GTAAGACAGGGGT | | | | |
| TRAC Cluster 32 | TGTGATGTCAAGCTGGTCGAGAAAAGCTTTGAAACAGGTAAGACAGGGG | 398 | T | E -> K | |
| | TCTAGCCTGGGTT | | | | |
| TRAC Cluster 33 | CCATAACCGCTGTGGCCTCTTGGTTTTACAGATACGAACCTAAACTTTC | 399 | A | Splice site | |
| | AAAACCTGTCAGT | | | | |
| TRAC Cluster 34 | TCTTGGTTTTACAGATACGAACCTAAACTTTCAAAACCTGTCAGTGATT | 400 | T | Q -> stop | |
| | GGGTTCCGAATCC | | | | |
| TRAC Cluster 35 | ACAGATACGAACCTAAACTTTCAAAACCTGTCAGTGATTGGGTTCCGAA | 401 | G | S -> L | |
| | TCCTCCTCCTGAA | | | | |
| TRAC Cluster 36 | ACTTTCAAAACCTGTCAGTGATTGGGTTCCGAATCCTCCTCCTGAAAGT | 402 | T | R -> N | Trans- |
| | GGCCGGGTTTAAT | | | | membrane |
| TRAC Cluster 37 | TTCAAAACCTGTCAGTGATTGGGTTCCGAATCCTCCTCCTGAAAGTGGC | 403 | A | L -> F | |
| | CGGGTTTAATCTG | | | | |
| TRAC Cluster 38 | AAAACCTGTCAGTGATTGGGTTCCGAATCCTCCTCCTGAAAGTGGCCGG | 404 | C | L -> F | |
| | GTTTAATCTGCTC | | | | |
| TRAC Cluster 39 | ACCTGTCAGTGATTGGGTTCCGAATCCTCCTCCTGAAAGTGGCCGGGTT | 405 | C | L -> F | |
| | TAATCTGCTCATG | | | | |
| TRAC Cluster 40 | CCTGAAAGTGGCCGGGTTTAATCTGCTCATGACGCTGCGGCTGTGGTCC | 406 | G | M -> I | |
| | AGCTGAGGTGAGG | | | | |
| TRAC Cluster 41 | TTTAATCTGCTCATGACGCTGCGGCTGTGGTCCAGCTGAGGTGAGGGGC | 407 | G | S -> F | |
| | CTTGAAGCTGGGA | | | | |

TABLE 8
List of TALEB target sequence windows following the rules of the present invention
to introduce mutations in the CD52 gene.

| | | | | Impact at | |
|---|---|---|---|---|---|
| | | | | trans- | |
| | | | | crip- | |
| | | SEQ | | tional/ | |
| Sequence | | ID | | trans- | |
| designation | Target window sequence in CD52 centered | NO | Base | lational | Protein |
| (CD52) | on TC/AG to be mutated (62 bp) | # | −1 | level | domain |
| CD52 Cluster 0 | CCAAGACAGCCACGAAGATCCTACCAAAATGAAGCGCTTCCTCTTCCT | 408 | T | M -> I | Signal s [symbol] |
| | CCTACTCACCATCA | | | | |
| CD52 Cluster 1 | GCCACGAAGATCCTACCAAAATGAAGCGCTTCCTCTTCCTCCTACTCA | 409 | T | L -> F | |
| | CCATCAGCCTCCTG | | | | |
| CD52 Cluster 2 | AAGATCCTACCAAAATGAAGCGCTTCCTCTTCCTCCTACTCACCATCA | 410 | T | L -> F | |
| | GCCTCCTGGTTATG | | | | |
| CD52 Cluster 3 | ATCCTACCAAAATGAAGCGCTTCCTCTTCCTCCTACTCACCATCAGCC | 411 | C | L -> F | |
| | TCCTGGTTATGGTA | | | | |
| CD52 Cluster 4 | GCTTCCTCTTCCTCCTACTCACCATCAGCCTCCTGGTTATGGTACAGG | 412 | C | L -> F | |
| | TAAGAGCAACGCCT | | | | |
| CD52 Cluster 5 | CCCTGATCTTATCCCACTTCTCCTCCTACAGATACAAACTGGACTCTC | 413 | A | Splice | |
| | AGGACAAAACGACA | | | site | |
| CD52 Cluster 6 | TCCCACTTCTCCTCCTACAGATACAAACTGGACTCTCAGGACAAAACG | 414 | G | G -> E | |
| | ACACCAGCCAAACC | | | | |
| CD52 Cluster 7 | CTTCTCCTCCTACAGATACAAACTGGACTCTCAGGACAAAACGACACC | 415 | C | S -> L | |
| | AGCCAAACCAGCAG | | | | |
| CD52 Cluster 8 | TCCTCCTACAGATACAAACTGGACTCTCAGGACAAAACGACACCAGCC | 416 | G | G -> E | CAMPATH- |
| | AAACCAGCAGCCCC | | | | 1 antigen |
| | | | | | binding |
| CD52 Cluster 9 | CAGATACAAACTGGACTCTCAGGACAAAACGACACCAGCCAAACCAGC | 417 | G | D -> N | |
| | AGCCCCTCAGCATC | | | | |
| CD52 Cluster 10 | CAAAACGACACCAGCCAAACCAGCAGCCCCTCAGCATCCAGCAACATA | 418 | C | S -> L | |
| | AGCGGAGGCATTTT | | | | |
| CD52 Cluster 11 | GACACCAGCCAAACCAGCAGCCCCTCAGCATCCAGCAACATAAGCGGA | 419 | A | S -> F | Removed |
| | GGCATTTTCCTTTT | | | | in |
| CD52 Cluster 12 | GCAGCCCCTCAGCATCCAGCAACATAAGCGGAGGCATTTTCCTTTTCT | 420 | C | G -> E | mature |
| | TCGTGGCCAATGCC | | | | form |
| CD52 Cluster 13 | CAGCATCCAGCAACATAAGCGGAGGCATTTTCCTTTTCTTCGTGGCCA | 421 | T | L -> F | |
| | ATGCCATAATCCAC | | | | |
| CD52 Cluster 14 | TTTTCCTTTTCTTCGTGGCCAATGCCATAATCCACCTCTTCTGCTTCA | 422 | A | H -> Y | |
| | GTTGAGGTGACACG | | | | |

[symbol]

indicates data missing or illegible when filed

TABLE 9
List of TALEB target sequence windows following the rules of the present invention
to introduce mutations in the PD1 gene.

| | | | | Impact | |
|---|---|---|---|---|---|
| | | | | at trans- | |
| | | | | crip- | |
| | | SEQ | | tional/ | |
| Sequence | | ID | | trans- | |
| designation | Target window sequence in PD1 centered | NO | Base | lational | Protein |
| (PD1) | on TC/AG to be mutated (62 bp) | # | −1 | level | domain |
| PD1 Cluster 0 | ACTCTGGTGGGGCTGCTCCAGGCATGCAGATCCCACAGGCGCCCTGGCC | 423 | A | P -> S | Signal se [symbol] |
| | AGTCGTCTGGGCG | | | | |
| PD1 Cluster 1 | GGGCGGTGCTACAACTGGGCTGGCGGCCAGGATGGTTCTTAGGTAGGTG | 424 | A | G -> E | |
| | GGGTCGGCGGTCA | | | | |
| PD1 Cluster 2 | AGCCCCTTCCTCACCTCTCTCCATCTCTCAGACTCCCCAGACAGGCCCT | 425 | G | Splice | |
| | GGAACCCCCCCAC | | | site | |
| PD1 Cluster 3 | CCCTTCCTCACCTCTCTCCATCTCTCAGACTCCCCAGACAGGCCCTGGA | 426 | C | S -> F | |
| | ACCCCCCCACCTT | | | | |
| PD1 Cluster 4 | CTCACCTCTCTCCATCTCTCAGACTCCCCAGACAGGCCCTGGAACCCCC | 427 | G | D -> N | |
| | CCACCTTCTCCCC | | | | |
| PD1 Cluster 5 | CCAGACAGGCCCTGGAACCCCCCCACCTTCTCCCCAGCCCTGCTCGTGG | 428 | C | S -> F | |
| | TGACCGAAGGGGA | | | | |
| PD1 Cluster 6 | ACCTTCTCCCCAGCCCTGCTCGTGGTGACCGAAGGGGACAACGCCACCT | 429 | T | E -> K | |
| | TCACCTGCAGCTT | | | | |
| PD1 Cluster 7 | TCCCCAGCCCTGCTCGTGGTGACCGAAGGGGACAACGCCACCTTCACCT | 430 | G | D -> N | |
| | GCAGCTTCTCCAA | | | | |
| PD1 Cluster 8 | GGGGACAACGCCACCTTCACCTGCAGCTTCTCCAACACATCGGAGAGCT | 431 | C | S -> F | |
| | TCGTGCTAAACTG | | | | |
| PD1 Cluster 9 | GCCACCTTCACCTGCAGCTTCTCCAACACATCGGAGAGCTTCGTGCTAA | 432 | A | S -> L | |
| | ACTGGTACCGCAT | | | | |
| PD1 Cluster 10 | ACCTTCACCTGCAGCTTCTCCAACACATCGGAGAGCTTCGTGCTAAACT | 433 | C | E -> K | |
| | GGTACCGCATGAG | | | | |
| PD1 Cluster 11 | GGAGAGCTTCGTGCTAAACTGGTACCGCATGAGCCCCAGCAACCAGACG | 434 | C | M -> I | Inter- |
| | GACAAGCTGGCCG | | | | action |
| | | | | | with |
| PD1 Cluster 12 | TGGTACCGCATGAGCCCCAGCAACCAGACGGACAAGCTGGCCGCCTTCC | 435 | G | D -> N | CD274/ |
| | CCGAGGACCGCAG | | | | PDCD1L1 |
| PD1 Cluster 13 | AACCAGACGGACAAGCTGGCCGCCTTCCCCGAGGACCGCAGCCAGCCCG | 436 | C | E -> K | |
| | GCCAGGACTGCCG | | | | |
| PD1 Cluster 14 | CAGACGGACAAGCTGGCCGCCTTCCCCGAGGACCGCAGCCAGCCCGGCC | 437 | G | D -> N | |
| | AGGACTGCCGCTT | | | | |
| PD1 Cluster 15 | TTCCCCGAGGACCGCAGCCAGCCCGGCCAGGACTGCCGCTTCCGTGTCA | 438 | G | D -> N | |
| | CACAACTGCCCAA | | | | |
| PD1 Cluster 16 | TTCCGTGTCACACAACTGCCCAACGGGCGTGACTTCCACATGAGCGTGG | 439 | G | D -> N | |
| | TCAGGGCCCGGCG | | | | |
| PD1 Cluster 17 | ACAACTGCCCAACGGGCGTGACTTCCACATGAGCGTGGTCAGGGCCCGG | 440 | C | M -> I | |
| | CGCAATGACAGCG | | | | |
| PD1 Cluster 18 | CACATGAGCGTGGTCAGGGCCCGGCGCAATGACAGCGGCACCTACCTCT | 441 | G | D -> N | |
| | GTGGGGCCATCTC | | | | |
| PD1 Cluster 19 | GACAGCGGCACCTACCTCTGTGGGGCCATCTCCCTGGCCCCCAAGGCGC | 442 | C | S -> F | |
| | AGATCAAAGAGAG | | | | |
| PD1 Cluster 20 | ATCTCCCTGGCCCCCAAGGCGCAGATCAAAGAGAGCCTGCGGGCAGAGC | 443 | C | E -> K | |
| | TCAGGGTGACAGG | | | | |
| PD1 Cluster 21 | GTCCTAACCCCTGACCTTTGTGCCCTTCCAGAGAGAAGGGCAGAAGTGC | 444 | C | Splice | |
| | CCACAGCCCACCC | | | site | |
| PD1 Cluster 22 | TAACCCCTGACCTTTGTGCCCTTCCAGAGAGAAGGGCAGAAGTGCCCAC | 445 | T | R -> K | |
| | AGCCCACCCCAGC | | | | |
| PD1 Cluster 23 | GACCTTTGTGCCCTTCCAGAGAGAAGGGCAGAAGTGCCCACAGCCCACC | 446 | T | E -> K | |
| | CCAGCCCCTCACC | | | | |
| PD1 Cluster 24 | GCAGAAGTGCCCACAGCCCACCCCAGCCCCTCACCCAGGCCAGCCGGCC | 447 | C | S -> F | |
| | AGTTCCAAACCCT | | | | |
| PD1 Cluster 25 | CTGCTAGTCTGGGTCCTGGCCGTCATCTGCTCCCGGGCCGCACGAGGTA | 448 | C | S -> F | |
| | ACGTCATCCCAGC | | | | |
| PD1 Cluster 26 | TCCTGGCCGTCATCTGCTCCCGGGCCGCACGAGGTAACGTCATCCCAGC | 449 | C | R -> N | |
| | CCCTCGGCCTGCC | | | | |
| PD1 Cluster 43 | CCCAAGTGTGTTTCTCTGCAGGGACAATAGGAGCCAGGCGCACCGGCCA | 450 | C | G -> E | |
| | GCCCCTGGTGAGT | | | | |
| PD1 Cluster 27 | GGGCTGACTCCCTCTCCCTTTCTCCTCAAAGAAGGAGGACCCCTCAGCC | 451 | T | Splice | |
| | GTGCCTGTGTTCT | | | site | |
| PD1 Cluster 28 | TGACTCCCTCTCCCTTTCTCCTCAAAGAAGGAGGACCCCTCAGCCGTGC | 452 | C | E -> K | |
| | CTGTGTTCTCTGT | | | | |
| PD1 Cluster 29 | CTCCCTTTCTCCTCAAAGAAGGAGGACCCCTCAGCCGTGCCTGTGTTCT | 453 | C | S -> L | |
| | CTGTGGACTATGG | | | | |
| PD1 Cluster 30 | AAGGAGGACCCCTCAGCCGTGCCTGTGTTCTCTGTGGACTATGGGGAGC | 454 | C | S -> F | |
| | TGGATTTCCAGTG | | | | |
| PD1 Cluster 31 | GCCGTGCCTGTGTTCTCTGTGGACTATGGGGAGCTGGATTTCCAGTGGC | 455 | C | E -> K | ITIM |
| | GAGAGAAGACCCC | | | | |
| PD1 Cluster 32 | GACTATGGGGAGCTGGATTTCCAGTGGCGAGAGAAGACCCCGGAGCCCC | 456 | C | E -> K | |
| | CCGTGCCCTGTGT | | | | |
| PD1 Cluster 33 | CTGGATTTCCAGTGGCGAGAGAAGACCCCGGAGCCCCCCGTGCCCTGTG | 457 | C | E -> K | |
| | TCCCTGAGCAGAC | | | | |
| PD1 Cluster 34 | ACCCCGGAGCCCCCCGTGCCCTGTGTCCCTGAGCAGACGGAGTATGCCA | 458 | C | E -> K | |
| | CCATTGTCTTTCC | | | | |
| PD1 Cluster 35 | CCCCCCGTGCCCTGTGTCCCTGAGCAGACGGAGTATGCCACCATTGTCT | 459 | C | E -> K | ITSM |
| | TTCCTAGCGGAAT | | | | |
| PD1 Cluster 36 | ACCATTGTCTTTCCTAGCGGAATGGGCACCTCATCCCCCGCCCGCAGGG | 460 | C | S -> L | |
| | GCTCAGCTGACGG | | | | |
| PD1 Cluster 37 | ATTGTCTTTCCTAGCGGAATGGGCACCTCATCCCCCGCCCGCAGGGGCT | 461 | A | S -> F | |
| | CAGCTGACGGCCC | | | | |
| PD1 Cluster 38 | ATGGGCACCTCATCCCCCGCCCGCAGGGGCTCAGCTGACGGCCCTCGGA | 462 | C | S -> L | |
| | GTGCCCAGCCACT | | | | |
| PD1 Cluster 39 | ACCTCATCCCCCGCCCGCAGGGGCTCAGCTGACGGCCCTCGGAGTGCCC | 463 | G | D -> N | |
| | AGCCACTGAGGCC | | | | |
| PD1 Cluster 40 | GCCCTCGGAGTGCCCAGCCACTGAGGCCTGAGGATGGACACTGCTCTTG | 464 | C | E -> K | |
| | GCCCCTCTGACCG | | | | |
| PD1 Cluster 41 | CCTCGGAGTGCCCAGCCACTGAGGCCTGAGGATGGACACTGCTCTTGGC | 465 | A | D -> N | |
| | CCCTCTGACCGGC | | | | |
| PD1 Cluster 42 | CAGCCACTGAGGCCTGAGGATGGACACTGCTCTTGGCCCCTCTGACCGG | 466 | C | S -> F | |
| | CTTCCTTGGCCAC | | | | |

[symbol]

indicates data missing or illegible when filed

TABLE 10
List of TALEB target sequence windows following the rules of the present invention
to introduce mutations in the B2m gene.

| | | | | Impact at | |
|---|---|---|---|---|---|
| Sequence | | | | transcriptional/ | |
| designation | Target window sequence in B2m centered on | SEQ ID | Base | translational | Protein |
| (B2m) | TC/AG to be mutated (62 bp) | NO # | −1 | level | domain |
| B2m Cluster 0 | GCTGACAGCATTCGGGCCGAGATGTCTCGCTCCGTGGCCTTAGCTGTGCTCGCGCTACTCTC | 467 | C | S->F | Signal |
| B2m Cluster 1 | TCCGTGGCCTTAGCTGTGCTCGCGCTACTCTCTCTTTCTGGCCTGGAGGCTATCCAGCGTGA | 468 | C | S->F | sequence |
| B2m Cluster 2 | CGTGGCCTTAGCTGTGCTCGCGCTACTCTCTCTTTCTGGCCTGGAGGCTATCCAGCGTGAGT | 469 | C | L->F | |
| B2m Cluster 3 | GCCTTAGCTGTGCTCGCGCTACTCTCTCTTTCTGGCCTGGAGGCTATCCAGCGTGAGTCTCT | 470 | T | S->F | |
| B2m Cluster 4 | GTGCTCGCGCTACTCTCTCTTTCTGGCCTGGAGGCTATCCAGCGTGAGTCTCTCCTACCCTC | 471 | C | E->K | |
| B2m Cluster 5 | TGTGTCTTTTCCCGATATTCCTCAGGTACTCCAAAGATTCAGGTTTACTCACGTCATCCAGC | 472 | C | P->S | |
| B2m Cluster 6 | TTCCCGATATTCCTCAGGTACTCCAAAGATTCAGGTTTACTCACGTCATCCAGCAGAGAATG | 473 | T | Q->stop | |
| B2m Cluster 7 | TCCTCAGGTACTCCAAAGATTCAGGTTTACTCACGTCATCCAGCAGAGAATGGAAAGTCAAA | 474 | C | S->L | |
| B2m Cluster 8 | TACTCCAAAGATTCAGGTTTACTCACGTCATCCAGCAGAGAATGGAAAGTCAAATTTCCTGA | 475 | A | P->S | |
| B2m Cluster 9 | AAGATTCAGGTTTACTCACGTCATCCAGCAGAGAATGGAAAGTCAAATTTCCTGAATTGCTA | 476 | C | E->K | |
| B2m Cluster 10 | TACTCACGTCATCCAGCAGAGAATGGAAAGTCAAATTTCCTGAATTGCTATGTGTCTGGGTT | 477 | G | S->L | |
| B2m Cluster 11 | GGAAAGTCAAATTTCCTGAATTGCTATGTGTCTGGGTTTCATCCATCCGACATTGAAGTTGA | 478 | G | S->F | |
| B2m Cluster 12 | AAATTTCCTGAATTGCTATGTGTCTGGGTTTCATCCATCCGACATTGAAGTTGACTTACTGA | 479 | T | H->Y | |
| B2m Cluster 13 | TTTCCTGAATTGCTATGTGTCTGGGTTTCATCCATCCGACATTGAAGTTGACTTACTGAAGA | 480 | A | P->S | |
| B2m Cluster 14 | CTGAATTGCTATGTGTCTGGGTTTCATCCATCCGACATTGAAGTTGACTTACTGAAGAATGG | 481 | A | S->F | |
| B2m Cluster 15 | TATGTGTCTGGGTTTCATCCATCCGACATTGAAGTTGACTTACTGAAGAATGGAGAGAGAAT | 482 | T | E->K | |
| B2m Cluster 16 | TCTGGGTTTCATCCATCCGACATTGAAGTTGACTTACTGAAGAATGGAGAGAGAATTGAAAA | 483 | G | D->N | |
| B2m Cluster 17 | GACATTGAAGTTGACTTACTGAAGAATGGAGAGAGAATTGAAAAAGTGGAGCATTCAGACTT | 484 | C | E->K | |
| B2m Cluster 18 | TTGAAGTTGACTTACTGAAGAATGGAGAGAGAATTGAAAAAGTGGAGCATTCAGACTTGTCT | 485 | T | R->K | |
| B2m Cluster 19 | GTTGACTTACTGAAGAATGGAGAGAGAATTGAAAAAGTGGAGCATTCAGACTTGTCTTTCAG | 486 | T | E->K | |
| B2m Cluster 20 | CTGAAGAATGGAGAGAGAATTGAAAAAGTGGAGCATTCAGACTTGTCTTTCAGCAAGGACTG | 487 | C | E->K | |
| B2m Cluster 21 | AATGGAGAGAGAATTGAAAAAGTGGAGCATTCAGACTTGTCTTTCAGCAAGGACTGGTCTTT | 488 | T | S->L | |
| B2m Cluster 22 | GGAGAGAGAATTGAAAAAGTGGAGCATTCAGACTTGTCTTTCAGCAAGGACTGGTCTTTCTA | 489 | G | D->N | |
| B2m Cluster 23 | AGAATTGAAAAAGTGGAGCATTCAGACTTGTCTTTCAGCAAGGACTGGTCTTTCTATCTCTT | 490 | G | S->F | |
| B2m Cluster 24 | GTGGAGCATTCAGACTTGTCTTTCAGCAAGGACTGGTCTTTCTATCTCTTGTACTACACTGA | 491 | G | D->N | |
| B2m Cluster 25 | CATTCAGACTTGTCTTTCAGCAAGGACTGGTCTTTCTATCTCTTGTACTACACTGAATTCAC | 492 | G | S->F | |
| B2m Cluster 26 | CTTGTCTTTCAGCAAGGACTGGTCTTTCTATCTCTTGTACTACACTGAATTCACCCCCACTG | 493 | A | L->F | |
| B2m Cluster 27 | GACTGGTCTTTCTATCTCTTGTACTACACTGAATTCACCCCCACTGAAAAAGATGAGTATGC | 494 | T | E->K | |
| B2m Cluster 28 | CTCTTGTACTACACTGAATTCACCCCCACTGAAAAAGATGAGTATGCCTGCCGTGTGAACCA | 495 | T | E->K | |
| B2m Cluster 29 | TACTACACTGAATTCACCCCCACTGAAAAAGATGAGTATGCCTGCCGTGTGAACCATGTGAC | 496 | A | D->N | D-> [symbol] |
| | | | | | AMY [symbol] |
| | | | | | reduced |
| | | | | | stability |
| B2m Cluster 30 | TACACTGAATTCACCCCCACTGAAAAAGATGAGTATGCCTGCCGTGTGAACCATGTGACTTT | 497 | C | E->K | |
| B2m Cluster 31 | TATGCCTGCCGTGTGAACCATGTGACTTTGTCACAGCCCAAGATAGTTAAGTGGGGTAAGTC | 498 | G | S->L | |
| B2m Cluster 32 | CTTTTTTTTCTCCACTGTCTTTTTCATAGATCGAGACATGTAAGCAGCATCATGGAGGTAAG | 499 | A | R->N | |
| B2m Cluster 33 | TTTTTTTCTCCACTGTCTTTTTCATAGATCGAGACATGTAAGCAGCATCATGGAGGTAAGTT | 500 | C | R->stop | |
| B2m Cluster 34 | TTTTTCTCCACTGTCTTTTTCATAGATCGAGACATGTAAGCAGCATCATGGAGGTAAGTTTT | 501 | G | D->N | |

[symbol]

indicates data missing or illegible when filed

TABLE 11
List of TALEB target sequence windows following the rules of the
present invention to introduce mutations in the ApoC3 gene.

| | | SEQ | | Impact at | |
|---|---|---|---|---|---|
| Sequence | | ID | | transcriptional/ | |
| designation | Target window sequence in APoC3 centered | NO | Base | translational | Proteins |
| (APoC3) | on TC/AG to be mutated (62 bp) | # | −1 | leve | domain |
| ApoC3 Cluster 0 | GGAACAGAGGTGCCATGCAGCCCCGGGTACTCCTTGTTGTTGCCCTCCTGGCGCTCCTGGCC | 502 | C | L->F | Signal |
| ApoC3 Cluster 1 | CTTGTTGTTGCCCTCCTGGCGCTCCTGGCCTCTGCCCGTAAGCACTTGGTGGGACTGGGCTG | 503 | C | S->F | seq |
| ApoC3 Cluster 2 | CCACCCCACTCAGCCCTGCTCTTTCCTCAGGAGCTTCAGAGGCCGAGGATGCCTCCCTTCTC | 504 | C | Splice site | |
| ApoC3 Cluster 3 | CCACTCAGCCCTGCTCTTTCCTCAGGAGCTTCAGAGGCCGAGGATGCCTCCCTTCTCAGCTT | 505 | T | S->L | |
| ApoC3 Cluster 4 | CTCAGCCCTGCTCTTTCCTCAGGAGCTTCAGAGGCCGAGGATGCCTCCCTTCTCAGCTTCAT | 506 | C | E->K | |
| ApoC3 Cluster 5 | TCCTCAGGAGCTTCAGAGGCCGAGGATGCCTCCCTTCTCAGCTTCATGCAGGGTTACATGAA | 507 | C | S->F | |
| ApoC3 Cluster 6 | AGGAGCTTCAGAGGCCGAGGATGCCTCCCTTCTCAGCTTCATGCAGGGTTACATGAAGCACG | 508 | T | L->F | |
| ApoC3 Cluster 7 | CTCCCTTCTCAGCTTCATGCAGGGTTACATGAAGCACGCCACCAAGACCGCCAAGGATGCAC | 509 | T | M->I | |
| ApoC3 Cluster 8 | TACATGAAGCACGCCACCAAGACCGCCAAGGATGCACTGAGCAGCGTGCAGGAGTCCCAGGT | 510 | A | D->N | |
| ApoC3 Cluster 9 | ACCGCCAAGGATGCACTGAGCAGCGTGCAGGAGTCCCAGGTGGCCCAGCAGGCCAGGTACAC | 511 | C | E->K | |
| ApoC3 Cluster 10 | GCCAAGGATGCACTGAGCAGCGTGCAGGAGTCCCAGGTGGCCCAGCAGGCCAGGTACACCCG | 512 | G | S->F | |
| ApoC3 Cluster 11 | TTTAGGGGCTGGGTGACCGATGGCTTCAGTTCCCTGAAAGACTACTGGAGCACCGTTAAGGA | 513 | T | S->F | Lipid |
| ApoC3 Cluster 12 | TGGGTGACCGATGGCTTCAGTTCCCTGAAAGACTACTGGAGCACCGTTAAGGACAAGTTCTC | 514 | G | D->N | binding |
| ApoC3 Cluster 13 | CGATGGCTTCAGTTCCCTGAAAGACTACTGGAGCACCGTTAAGGACAAGTTCTCTGAGTTCT | 515 | C | W->stop | |
| ApoC3 Cluster 14 | TCCCTGAAAGACTACTGGAGCACCGTTAAGGACAAGTTCTCTGAGTTCTGGGATTTGGACCC | 516 | G | D->N | |
| ApoC3 Cluster 15 | GACTACTGGAGCACCGTTAAGGACAAGTTCTCTGAGTTCTGGGATTTGGACCCTGAGGTCAG | 517 | C | S->F | |
| ApoC3 Cluster 16 | TACTGGAGCACCGTTAAGGACAAGTTCTCTGAGTTCTGGGATTTGGACCCTGAGGTCAGACC | 518 | C | E->K | |
| ApoC3 Cluster 17 | ACCGTTAAGGACAAGTTCTCTGAGTTCTGGGATTTGGACCCTGAGGTCAGACCAACTTCAGC | 519 | A | D->N | |
| ApoC3 Cluster 18 | AAGGACAAGTTCTCTGAGTTCTGGGATTTGGACCCTGAGGTCAGACCAACTTCAGCCGTGGC | 520 | G | D->N | |
| ApoC3 Cluster 19 | AAGTTCTCTGAGTTCTGGGATTTGGACCCTGAGGTCAGACCAACTTCAGCCGTGGCTGCCTG | 521 | C | E->K | |
| ApoC3 Cluster 20 | CTGAGTTCTGGGATTTGGACCCTGAGGTCAGACCAACTTCAGCCGTGGCTGCCTGAGACCTC | 522 | G | R->K | |
| ApoC3 Cluster 21 | TGGGATTTGGACCCTGAGGTCAGACCAACTTCAGCCGTGGCTGCCTGAGACCTCAATACCCC | 523 | T | S->L | |

TABLE 12
Base editors target sites in Exon 1, 2 or 3 of PK13 gene as per the combined gene therapy method illustrated in example 5.

| Exons | | | SEQ | | | | | | |
|---|---|---|---|---|---|---|---|---|---|
| of | Genomic | | ID | Binding Site | | | | | |
| PI3KCD | region | Targeted sequence | # | bp | LEFT Binding site | bp | SPACER | bp | RIGHT Binding site |
| Exon 1 | splice | TGGAAAAGCCCGGCCTGCACCACCAGCTGTAGAAGGTGCCGGGA | 524 | 16 | TGGAAAAGCCCGGCCT | 14 | GCACCACCAGCTGT | 14 | AGAAGGTGCCGGGA |
| | site | TGGAAAAGCCCGGCCTGCACCACCAGCTGTAGAAGGTGCCGGGATGA | 525 | 16 | TGGAAAAGCCCGGCCT | 14 | GCACCACCAGCTGT | 17 | AGAAGGTGCCGGGA A |
| | | TGGAAAAGCCCGGCCTGCACCACCAGCTGTAGAAGGTGCCGGGATGA | 526 | 16 | TGGAAAAGCCCGGCCT | 15 | GCACCACCAGCTGTA | 16 | GAAGGTGCCGGGATGA |
| | stop | TGATGTCGAACTCCAGCCGCTGCTTCCACACGGGCTCCGAGCACA | 527 | 14 | TGATGTCGAACTCC | 15 | AGCCGCTGCTTCCAC | 16 | ACGGGCTCCGAGCACA |
| | codon- | TTGATGTCGAACTCCAGCCGCTGCTTCCACACGGGCTCCGAGCACA | 528 | 15 | TTGATGTCGAACTCC | 15 | AGCCGCTGCTTCCAC | 16 | ACGGGCTCCGAGCACA |
| | strand | TGTTGATGTCGAACTCCAGCCGCTGCTTCCACACGGGCTCCGAGCACA | 529 | 17 | TGTTGATGTCGAACTCC | 15 | AGCCGCTGCTTCCAC | 16 | ACGGGCTCCGAGCACA |
| | | TGATGTCGAACTCCAGCCGCTGCTTCCACACGGGCTCCGAGCA | 530 | 14 | TGATGTCGAACTCC | 15 | AGCCGCTGCTTCCAC | 14 | ACGGGCTCCGAGCA |
| | | TGCTCGGAGCCCGTGTGGAAGCAGCGGCTGGAGTTCGACATCAA | 531 | 15 | TGCTCGGAGCCCGTG | 15 | TGGAAGCAGCGGCTG | 14 | GAGTTCGACATCAA |
| | | TGTTGATGTCGAACTCCAGCCGCTGCTTCCACACGGGCTCCGAGCA | 532 | 17 | TGTTGATGTCGAACTCC | 15 | AGCCGCTGCTTCCAC | 14 | ACGGGCTCCGAGCA |
| Exon 2 | splice | TGAGGTTGGCCCAGGCAATGGGGCAGTCCTGCAGAAGGACAGGGCA | 533 | 17 | TGAGGTTGGCCCAGGCA | 15 | ATGGGGCAGTCCTGC | 14 | AGAAGGACAGGGCA |
| | site | TTGGCCCAGGCAATGGGGCAGTCCTGCAGAAGGACAGGGCA | 534 | 15 | TTGGCCCAGGCAATG | 12 | GGGCAGTCCTGC | 14 | AGAAGGACAGGGCA |
| | | TTGGCCCAGGCAATGGGGCAGTCCTGCAGAAGGACAGGGCAGGTGA | 535 | 15 | TTGGCCCAGGCAATG | 14 | GGGCAGTCCTGCAG | 17 | AAGGACAGGGCAGGTGA |
| | | TTGGCCCAGGCAATGGGGCAGTCCTGCAGAAGGACAGGGCAGGTGA | 536 | 16 | TTGGCCCAGGCAATGG | 13 | GGCAGTCCTGCAG | 17 | AAGGACAGGGCAGGTGA |
| | stop | TTGTAGTCAAACAGCATGAGGTTGGCCCAGGCAATGGGGCAGTCCTGCA | 537 | 17 | TTGTAGTCAAACAGCAT | 15 | GAGGTTGGCCCAGGC | 17 | AATGGGGCAGTCCTGCA |
| | codon- | TGTAGTCAAACAGCATGAGGTTGGCCCAGGCAATGGGGCAGTCCTGCA | 538 | 16 | TGTAGTCAAACAGCAT | 15 | GAGGTTGGCCCAGGC | 17 | AATGGGGCAGTCCTGCA |
| | strand | TAGTCAAACAGCATGAGGTTGGCCCAGGCAATGGGGCAGTCCTGCA | 539 | 16 | TAGTCAAACAGCATGA | 13 | GGTTGGCCCAGGC | 17 | AATGGGGCAGTCCTGCA |
| Exon 3 | splice | TGGGGTTCAGCAGCTCGCCCTTCTCATCTGAACACAGGGGCAGATGAA | 540 | 16 | TGGGGTTCAGCAGCTC | 15 | GCCCTTCTCATCTGA | 17 | ACACAGGGGCAGATGAA |
| | site | TCAGCAGCTCGCCCTTCTCATCTGAACACAGGGGCAGATGAA | 541 | 13 | TCAGCAGCTCGCC | 12 | CTTCTCATCTGA | 17 | ACACAGGGGCAGAT A |
| | | TGGGGTTCAGCAGCTCGCCCTTCTCATCTGAACACAGGGGCAGATGAA | 542 | 17 | TGGGGTTCAGCAGCTCG | 14 | CCCTTCTCATCTGA | 17 | ACACAGGGGCAGAT A |
| | | TCAGCAGCTCGCCCTTCTCATCTGAACACAGGGGCAGATGAA | 543 | 13 | TCAGCAGCTCGCC | 13 | CTTCTCATCTGAA | 16 | CACAGGGGCAG |
| | | TGGGGTTCAGCAGCTCGCCCTTCTCATCTGAACACAGGGGCAGATGAA | 544 | 17 | TGGGGTTCAGCAGCTCG | 15 | CCCTTCTCATCTGAA | 16 | CACAGGGGCAG. |
| | splice | TGGGGTTCAGCAGCTCGCCCTTCTCATCTGAACACAGGGGCAGATGAA | 545 | 16 | TGGGGTTCAGCAGCTC | 15 | GCCCTTCTCATCTGA | 17 | ACACAGGGGCA( |
| | site- | TGGGGTTCAGCAGCTCGCCCTTCTCATCTGAACACAGGGGCAGATGAA | 546 | 17 | TGGGGTTCAGCAGCTCG | 15 | CCCTTCTCATCTGAA | 16 | CACAGGGGCAGATG |
| | strand | TGGGGTTCAGCAGCTCGCCCTTCTCATCTGAACACAGGGGCAGATGAA | 547 | 17 | TGGGGTTCAGCAGCTCG | 14 | CCCTTCTCATCTGA | 17 | ACACAGGGGCAGAT |
| | | TGGGGTTCAGCAGCTCGCCCTTCTCATCTGAACACAGGGGCAGATGAA | 548 | 17 | TGGGGTTCAGCAGCTCG | 15 | CCCTTCTCATCTGAA | 16 | CACAGGGGCAGATG |
| | | TCAGCAGCTCGCCCTTCTCATCTGAACACAGGGGCAGATGAA | 549 | 13 | TCAGCAGCTCGCC | 12 | CTTCTCATCTGA | 17 | ACACAGGGGCAGAT |
| | | TCAGCAGCTCGCCCTTCTCATCTGAACACAGGGGCAGATGAA | 550 | 13 | TCAGCAGCTCGCC | 13 | CTTCTCATCTGAA | 16 | CACAGGGGCAGATG |

TABLE 15
Initial STAT3 TALE target sequence library spanning from 5 to 17 bp

| Target | SEQ | |
|---|---|---|
| name | ID # | Polynucleotide sequence |
| TCGA_1 | 568 | GAGATCATGTCCTAACCCTGATCCTCTTGTCCCACAGATATCCAGAACCCTCTAAGAAGTTCCTGCTTCGATGAATGTGGTTAGAGAC |
| | | AAAACAGTGACCCTGCCGTGTACCAGCTGAGAGACTCTAAATCCAGTGACAAGTCTG |
| TCGA_2 | 569 | GAGATCATGTCCTAACCCTGATCCTCTTGTCCCACAGATATCCAGAACCCTCTAAGAAGTTCCTGCTATCGAGAATGTGGTTAGAGAC |
| | | AAAACCTTGACCCTGCCGTGTACCAGCTGAGAGACTCTAAATCCAGTGACAAGTCTG |
| TCGA_3 | 570 | GAGATCATGTCCTAACCCTGATCCTCTTGTCCCACAGATATCCAGAACCCTCTAAGAAGTTCCTGCTAATCGGAATGTGGTTAGAGAC |
| | | AAAACGATGACCCTGCCGTGTACCAGCTGAGAGACTCTAAATCCAGTGACAAGTCTG |
| TCGA_4 | 571 | GAGATCATGTCCTAACCCTGATCCTCTTGTCCCACAGATATCCAGAACCCTCTAAGAAGTTCCTGCTGAATCGAATGTGGTTAGAGAC |
| | | AAAACGGTGACCCTGCCGTGTACCAGCTGAGAGACTCTAAATCCAGTGACAAGTCTG |
| TCGA_5 | 572 | GAGATCATGTCCTAACCCTGATCCTCTTGTCCCACAGATATCCAGAACCCTCTAAGAAGTTCCTGCTTCGATATGAATGTGGTTAGAG |
| | | ACAAAACTCTGACCCTGCCGTGTACCAGCTGAGAGACTCTAAATCCAGTGACAAGTCTG |
| TCGA_6 | 573 | GAGATCATGTCCTAACCCTGATCCTCTTGTCCCACAGATATCCAGAACCCTCTAAGAAGTTCCTGCTAATCGATGAATGTGGTTAGAG |
| | | ACAAAAGACTGACCCTGCCGTGTACCAGCTGAGAGACTCTAAATCCAGTGACAAGTCTG |
| TCGA_7 | 574 | GAGATCATGTCCTAACCCTGATCCTCTTGTCCCACAGATATCCAGAACCCTCTAAGAAGTTCCTGCTGATATCAGAATGTGGTTAGAG |
| | | ACAAAAGAGTGACCCTGCCGTGTACCAGCTGAGAGACTCTAAATCCAGTGACAAGTCTG |
| TCGA_8 | 575 | GAGATCATGTCCTAACCCTGATCCTCTTGTCCCACAGATATCCAGAACCCTCTAAGAAGTTCCTGCTTCGAtattaGAATGTGGTTAGAG |
| | | ACAAAAGCTTGACCCTGCCGTGTACCAGCTGAGAGACTCTAAATCCAGTGACAAGTCTG |
| TCGA_9 | 576 | GAGATCATGTCCTAACCCTGATCCTCTTGTCCCACAGATATCCAGAACCCTCTAAGAAGTTCCTGCTtaTCGAtatGAATGTGGTTAGAG |
| | | ACAAAAGGATGACCCTGCCGTGTACCAGCTGAGAGACTCTAAATCCAGTGACAAGTCTG |
| TCGA_10 | 577 | GAGATCATGTCCTAACCCTGATCCTCTTGTCCCACAGATATCCAGAACCCTCTAAGAAGTTCCTGCTattaTCGAtGAATGTGGTTAGAG |
| | | ACAAAAGGGTGACCCTGCCGTGTACCAGCTGAGAGACTCTAAATCCAGTGACAAGTCTG |
| TCGA_11 | 578 | GAGATCATGTCCTAACCCTGATCCTCTTGTCCCACAGATATCCAGAACCCTCTAAGAAGTTCCTGCTAtattaTCGGAATGTGGTTAGAG |
| | | ACAAAAGTCTGACCCTGCCGTGTACCAGCTGAGAGACTCTAAATCCAGTGACAAGTCTG |
| TCGA_12 | 579 | GAGATCATGTCCTAACCCTGATCCTCTTGTCCCACAGATATCCAGAACCCTCTAAGAAGTTCCTGCTTCGAtatttaaGAATGTGGTTAGA |
| | | GACAAAATCCTGACCCTGCCGTGTACCAGCTGAGAGACTCTAAATCCAGTGACAAGTCTG |
| TCGA_13 | 580 | GAGATCATGTCCTAACCCTGATCCTCTTGTCCCACAGATATCCAGAACCCTCTAAGAAGTTCCTGCTaaTCGAtatttGAATGTGGTTAGA |
| | | GACAAAATGCTGACCCTGCCGTGTACCAGCTGAGAGACTCTAAATCCAGTGACAAGTCTG |
| TCGA_14 | 581 | GAGATCATGTCCTAACCCTGATCCTCTTGTCCCACAGATATCCAGAACCCTCTAAGAAGTTCCTGCTttaaTCGAtatGAATGTGGTTAGA |
| | | GACAAACAAGTGACCCTGCCGTGTACCAGCTGAGAGACTCTAAATCCAGTGACAAGTCTG |
| TCGA_15 | 582 | GAGATCATGTCCTAACCCTGATCCTCTTGTCCCACAGATATCCAGAACCCTCTAAGAAGTTCCTGCTatttaaTCGAtGAATGTGGTTAGA |
| | | GACAAACACCTGACCCTGCCGTGTACCAGCTGAGAGACTCTAAATCCAGTGACAAGTCTG |
| TCGA_16 | 583 | GAGATCATGTCCTAACCCTGATCCTCTTGTCCCACAGATATCCAGAACCCTCTAAGAAGTTCCTGCTAtatttaaTCGGAATGTGGTTAGA |
| | | GACAAACAGATGACCCTGCCGTGTACCAGCTGAGAGACTCTAAATCCAGTGACAAGTCTG |
| TCGA_17 | 584 | GAGATCATGTCCTAACCCTGATCCTCTTGTCCCACAGATATCCAGAACCCTCTAAGAAGTTCCTGCTTCGAtaattataaGAATGTGGTTA |
| | | GAGACAAACAGGTGACCCTGCCGTGTACCAGCTGAGAGACTCTAAATCCAGTGACAAGTCTG |
| TCGA_18 | 585 | GAGATCATGTCCTAACCCTGATCCTCTTGTCCCACAGATATCCAGAACCCTCTAAGAAGTTCCTGCTaaTCGAtaattatGAATGTGGTTA |
| | | GAGACAAACCATTGACCCTGCCGTGTACCAGCTGAGAGACTCTAAATCCAGTGACAAGTCTG |
| TCGA_19 | 586 | GAGATCATGTCCTAACCCTGATCCTCTTGTCCCACAGATATCCAGAACCCTCTAAGAAGTTCCTGCTataaTCGAtaattGAATGTGGTTA |
| | | GAGACAAACCGTTGACCCTGCCGTGTACCAGCTGAGAGACTCTAAATCCAGTGACAAGTCTG |
| TCGA_20 | 587 | GAGATCATGTCCTAACCCTGATCCTCTTGTCCCACAGATATCCAGAACCCTCTAAGAAGTTCCTGCTttataaTCGAtaaGAATGTGGTTA |
| | | GAGACAAACGCATGACCCTGCCGTGTACCAGCTGAGAGACTCTAAATCCAGTGACAAGTCTG |
| TCGA_21 | 588 | GAGATCATGTCCTAACCCTGATCCTCTTGTCCCACAGATATCCAGAACCCTCTAAGAAGTTCCTGCTaattataaTCGAtGAATGTGGTTA |
| | | GAGACAAACTCGTGACCCTGCCGTGTACCAGCTGAGAGACTCTAAATCCAGTGACAAGTCTG |
| TCGA_22 | 589 | GAGATCATGTCCTAACCCTGATCCTCTTGTCCCACAGATATCCAGAACCCTCTAAGAAGTTCCTGCTAtaattataaTCGGAATGTGGTTA |
| | | GAGACAAACTGGTGACCCTGCCGTGTACCAGCTGAGAGACTCTAAATCCAGTGACAAGTCTG |
| TCGA_23 | 590 | GAGATCATGTCCTAACCCTGATCCTCTTGTCCCACAGATATCCAGAACCCTCTAAGAAGTTCCTGCTTCGAttataattaaaGAATGTGGTT |
| | | AGAGACAAACTTCTGACCCTGCCGTGTACCAGCTGAGAGACTCTAAATCCAGTGACAAGTCTG |
| TCGA_24 | 591 | GAGATCATGTCCTAACCCTGATCCTCTTGTCCCACAGATATCCAGAACCCTCTAAGAAGTTCCTGCTaaTCGAttataattaGAATGTGGTT |
| | | AGAGACAAAGAACTGACCCTGCCGTGTACCAGCTGAGAGACTCTAAATCCAGTGACAAGTCTG |
| TCGA_25 | 592 | GAGATCATGTCCTAACCCTGATCCTCTTGTCCCACAGATATCCAGAACCCTCTAAGAAGTTCCTGCTtaaaTCGAttataatGAATGTGGTT |
| | | AGAGACAAAGAAGTGACCCTGCCGTGTACCAGCTGAGAGACTCTAAATCCAGTGACAAGTCTG |
| TCGA_26 | 593 | GAGATCATGTCCTAACCCTGATCCTCTTGTCCCACAGATATCCAGAACCCTCTAAGAAGTTCCTGCTattaaaTCGAttataGAATGTGGTT |
| | | AGAGACAAAGACATGACCCTGCCGTGTACCAGCTGAGAGACTCTAAATCCAGTGACAAGTCTG |
| TCGA_27 | 594 | GAGATCATGTCCTAACCCTGATCCTCTTGTCCCACAGATATCCAGAACCCTCTAAGAAGTTCCTGCTtaattaaaTCGAttaGAATGTGGTT |
| | | AGAGACAAAGACTTGACCCTGCCGTGTACCAGCTGAGAGACTCTAAATCCAGTGACAAGTCTG |
| TCGA_28 | 595 | GAGATCATGTCCTAACCCTGATCCTCTTGTCCCACAGATATCCAGAACCCTCTAAGAAGTTCCTGCTtataattaaaTCGAtGAATGTGGTT |
| | | AGAGACAAAGAGATGACCCTGCCGTGTACCAGCTGAGAGACTCTAAATCCAGTGACAAGTCTG |
| TCGA_29 | 596 | GAGATCATGTCCTAACCCTGATCCTCTTGTCCCACAGATATCCAGAACCCTCTAAGAAGTTCCTGCTAttataattaaaTCGGAATGTGGTT |
| | | AGAGACAAAGAGTTGACCCTGCCGTGTACCAGCTGAGAGACTCTAAATCCAGTGACAAGTCTG |
| TCGA_30 | 597 | GAGATCATGTCCTAACCCTGATCCTCTTGTCCCACAGATATCCAGAACCCTCTAAGAAGTTCCTGCTTCGAtattattaaattaGAATGTGGT |
| | | TAGAGACAAAGATCTGACCCTGCCGTGTACCAGCTGAGAGACTCTAAATCCAGTGACAAGTCTG |
| TCGA_31 | 598 | GAGATCATGTCCTAACCCTGATCCTCTTGTCCCACAGATATCCAGAACCCTCTAAGAAGTTCCTGCTtaTCGAtattattaaatGAATGTGGT |
| | | TAGAGACAAAGCTGTGACCCTGCCGTGTACCAGCTGAGAGACTCTAAATCCAGTGACAAGTCTG |
| TCGA_32 | 599 | GAGATCATGTCCTAACCCTGATCCTCTTGTCCCACAGATATCCAGAACCCTCTAAGAAGTTCCTGCTattaTCGAtattattaaGAATGTGGT |
| | | TAGAGACAAAGGAATGACCCTGCCGTGTACCAGCTGAGAGACTCTAAATCCAGTGACAAGTCTG |
| TCGA_33 | 600 | GAGATCATGTCCTAACCCTGATCCTCTTGTCCCACAGATATCCAGAACCCTCTAAGAAGTTCCTGCTaaattaTCGAtattattGAATGTGGT |
| | | TAGAGACAAAGGAGTGACCCTGCCGTGTACCAGCTGAGAGACTCTAAATCCAGTGACAAGTCTG |
| TCGA_34 | 601 | GAGATCATGTCCTAACCCTGATCCTCTTGTCCCACAGATATCCAGAACCCTCTAAGAAGTTCCTGCTttaaattaTCGAtattaGAATGTGGT |
| | | TAGAGACAAAGGGATGACCCTGCCGTGTACCAGCTGAGAGACTCTAAATCCAGTGACAAGTCTG |
| TCGA_35 | 602 | GAGATCATGTCCTAACCCTGATCCTCTTGTCCCACAGATATCCAGAACCCTCTAAGAAGTTCCTGCTtattaaattaTCGAtatGAATGTGGT |
| | | TAGAGACAAAGGGTTGACCCTGCCGTGTACCAGCTGAGAGACTCTAAATCCAGTGACAAGTCTG |
| TCGA_36 | 603 | GAGATCATGTCCTAACCCTGATCCTCTTGTCCCACAGATATCCAGAACCCTCTAAGAAGTTCCTGCTattattaaattaTCGAtGAATGTGGT |
| | | TAGAGACAAAGGTCTGACCCTGCCGTGTACCAGCTGAGAGACTCTAAATCCAGTGACAAGTCTG |
| TCGA_37 | 604 | GAGATCATGTCCTAACCCTGATCCTCTTGTCCCACAGATATCCAGAACCCTCTAAGAAGTTCCTGCTAtattattaaattaTCGGAATGTGGT |
| | | TAGAGACAAAGGTGTGACCCTGCCGTGTACCAGCTGAGAGACTCTAAATCCAGTGACAAGTCTG |

TABLE 16
library comprising 256 target sequences (ssODN)
with 15 bp spacers designed to test TC context in Example 6

| | SEQ | |
|---|---|---|
| Name | ID # | Polynucleotide arget sequence |
| TC15P1_pool1_1 | 605 | GAGATCATGTCCTAACCCTGATCCTCTTGTCCCACAGATATCCAGAACCCTCTAAGAAGTTCCTGCTTTTAAAATAATCAAAGAA |
| | | TGTGGTTAGAGACATGACCCTGCCGTGTACCAGCTGAGAGACTCTAAATCCAGTGACAAGTCTG |
| TC15P1_pool1_2 | 606 | GAGATCATGTCCTAACCCTGATCCTCTTGTCCCACAGATATCCAGAACCCTCTAAGAAGTTCCTGCTATTTTAATAATCACTGAA |
| | | TGTGGTTAGAGACATGACCCTGCCGTGTACCAGCTGAGAGACTCTAAATCCAGTGACAAGTCTG |
| TC15P1_pool1_3 | 607 | GAGATCATGTCCTAACCCTGATCCTCTTGTCCCACAGATATCCAGAACCCTCTAAGAAGTTCCTGCTATAAATAAAATCAGAGA |
| | | ATGTGGTTAGAGACATGACCCTGCCGTGTACCAGCTGAGAGACTCTAAATCCAGTGACAAGTCTG |
| TC15P1_pool1_4 | 608 | GAGATCATGTCCTAACCCTGATCCTCTTGTCCCACAGATATCCAGAACCCTCTAAGAAGTTCCTGCTTTATATTTAATCATAGAA |
| | | TGTGGTTAGAGACATGACCCTGCCGTGTACCAGCTGAGAGACTCTAAATCCAGTGACAAGTCTG |
| TC15P1_pool1_5 | 609 | GAGATCATGTCCTAACCCTGATCCTCTTGTCCCACAGATATCCAGAACCCTCTAAGAAGTTCCTGCTAATATATTAATCCAAGAA |
| | | TGTGGTTAGAGACATGACCCTGCCGTGTACCAGCTGAGAGACTCTAAATCCAGTGACAAGTCTG |
| TC15P1_pool1_6 | 610 | GAGATCATGTCCTAACCCTGATCCTCTTGTCCCACAGATATCCAGAACCCTCTAAGAAGTTCCTGCTTTTAAATAAATCCCAGA |
| | | ATGTGGTTAGAGACATGACCCTGCCGTGTACCAGCTGAGAGACTCTAAATCCAGTGACAAGTCTG |
| TC15P1_pool1_7 | 611 | GAGATCATGTCCTAACCCTGATCCTCTTGTCCCACAGATATCCAGAACCCTCTAAGAAGTTCCTGCTTTAAAAAAAATCCGAGA |
| | | ATGTGGTTAGAGACATGACCCTGCCGTGTACCAGCTGAGAGACTCTAAATCCAGTGACAAGTCTG |
| TC15P1_pool1_8 | 612 | GAGATCATGTCCTAACCCTGATCCTCTTGTCCCACAGATATCCAGAACCCTCTAAGAAGTTCCTGCTTATTATTTAATCCTTGAA |
| | | TGTGGTTAGAGACATGACCCTGCCGTGTACCAGCTGAGAGACTCTAAATCCAGTGACAAGTCTG |
| TC15P1_pool1_9 | 613 | GAGATCATGTCCTAACCCTGATCCTCTTGTCCCACAGATATCCAGAACCCTCTAAGAAGTTCCTGCTTTATTTAAAATCGATGAA |
| | | TGTGGTTAGAGACATGACCCTGCCGTGTACCAGCTGAGAGACTCTAAATCCAGTGACAAGTCTG |
| TC15P1_pool1_10 | 614 | GAGATCATGTCCTAACCCTGATCCTCTTGTCCCACAGATATCCAGAACCCTCTAAGAAGTTCCTGCTAAAATAATAATCGCAGA |
| | | ATGTGGTTAGAGACATGACCCTGCCGTGTACCAGCTGAGAGACTCTAAATCCAGTGACAAGTCTG |
| TC15P1_pool1_11 | 615 | GAGATCATGTCCTAACCCTGATCCTCTTGTCCCACAGATATCCAGAACCCTCTAAGAAGTTCCTGCTATATTAAAAATCGGTGA |
| | | ATGTGGTTAGAGACATGACCCTGCCGTGTACCAGCTGAGAGACTCTAAATCCAGTGACAAGTCTG |
| TC15P1_pool1_12 | 616 | GAGATCATGTCCTAACCCTGATCCTCTTGTCCCACAGATATCCAGAACCCTCTAAGAAGTTCCTGCTATTAATATAATCGTAGAA |
| | | TGTGGTTAGAGACATGACCCTGCCGTGTACCAGCTGAGAGACTCTAAATCCAGTGACAAGTCTG |
| TC15P1_pool1_13 | 617 | GAGATCATGTCCTAACCCTGATCCTCTTGTCCCACAGATATCCAGAACCCTCTAAGAAGTTCCTGCTATTATATTAATCTATGAA |
| | | TGTGGTTAGAGACATGACCCTGCCGTGTACCAGCTGAGAGACTCTAAATCCAGTGACAAGTCTG |
| TC15P1_pool1_14 | 618 | GAGATCATGTCCTAACCCTGATCCTCTTGTCCCACAGATATCCAGAACCCTCTAAGAAGTTCCTGCTATATATTTAATCTCTGAA |
| | | TGTGGTTAGAGACATGACCCTGCCGTGTACCAGCTGAGAGACTCTAAATCCAGTGACAAGTCTG |
| TC15P1_pool1_15 | 619 | GAGATCATGTCCTAACCCTGATCCTCTTGTCCCACAGATATCCAGAACCCTCTAAGAAGTTCCTGCTAAAATATAAATCTGTGA |
| | | ATGTGGTTAGAGACATGACCCTGCCGTGTACCAGCTGAGAGACTCTAAATCCAGTGACAAGTCTG |
| TC15P1_pool1_16 | 620 | GAGATCATGTCCTAACCCTGATCCTCTTGTCCCACAGATATCCAGAACCCTCTAAGAAGTTCCTGCTTATAATAAAATCTTTGAA |
| | | TGTGGTTAGAGACATGACCCTGCCGTGTACCAGCTGAGAGACTCTAAATCCAGTGACAAGTCTG |
| TC15P1_pool1_17 | 621 | GAGATCATGTCCTAACCCTGATCCTCTTGTCCCACAGATATCCAGAACCCTCTAAGAAGTTCCTGCTTAATAAATACTCAATGAA |
| | | TGTGGTTAGAGACATGACCCTGCCGTGTACCAGCTGAGAGACTCTAAATCCAGTGACAAGTCTG |
| TC15P1_pool1_18 | 622 | GAGATCATGTCCTAACCCTGATCCTCTTGTCCCACAGATATCCAGAACCCTCTAAGAAGTTCCTGCTAATAATAAACTCACAGA |
| | | ATGTGGTTAGAGACATGACCCTGCCGTGTACCAGCTGAGAGACTCTAAATCCAGTGACAAGTCTG |
| TC15P1_pool1_19 | 623 | GAGATCATGTCCTAACCCTGATCCTCTTGTCCCACAGATATCCAGAACCCTCTAAGAAGTTCCTGCTATAATAATACTCAGTGA |
| | | ATGTGGTTAGAGACATGACCCTGCCGTGTACCAGCTGAGAGACTCTAAATCCAGTGACAAGTCTG |
| TC15P1_pool1_20 | 624 | GAGATCATGTCCTAACCCTGATCCTCTTGTCCCACAGATATCCAGAACCCTCTAAGAAGTTCCTGCTTTATTAAAACTCATAGAA |
| | | TGTGGTTAGAGACATGACCCTGCCGTGTACCAGCTGAGAGACTCTAAATCCAGTGACAAGTCTG |
| TC15P1_pool1_21 | 625 | GAGATCATGTCCTAACCCTGATCCTCTTGTCCCACAGATATCCAGAACCCTCTAAGAAGTTCCTGCTAATTATTTACTCCAAGAA |
| | | TGTGGTTAGAGACATGACCCTGCCGTGTACCAGCTGAGAGACTCTAAATCCAGTGACAAGTCTG |
| TC15P1_pool1_22 | 626 | GAGATCATGTCCTAACCCTGATCCTCTTGTCCCACAGATATCCAGAACCCTCTAAGAAGTTCCTGCTAAATAATTACTCCCAGA |
| | | ATGTGGTTAGAGACATGACCCTGCCGTGTACCAGCTGAGAGACTCTAAATCCAGTGACAAGTCTG |
| TC15P1_pool1_23 | 627 | GAGATCATGTCCTAACCCTGATCCTCTTGTCCCACAGATATCCAGAACCCTCTAAGAAGTTCCTGCTTATTTAATACTCCGTGA |
| | | ATGTGGTTAGAGACATGACCCTGCCGTGTACCAGCTGAGAGACTCTAAATCCAGTGACAAGTCTG |
| TC15P1_pool1_24 | 628 | GAGATCATGTCCTAACCCTGATCCTCTTGTCCCACAGATATCCAGAACCCTCTAAGAAGTTCCTGCTTTAATTTAACTCCTAGAA |
| | | TGTGGTTAGAGACATGACCCTGCCGTGTACCAGCTGAGAGACTCTAAATCCAGTGACAAGTCTG |
| TC15P1_pool1_25 | 629 | GAGATCATGTCCTAACCCTGATCCTCTTGTCCCACAGATATCCAGAACCCTCTAAGAAGTTCCTGCTAATTTAAAACTCGATGA |
| | | ATGTGGTTAGAGACATGACCCTGCCGTGTACCAGCTGAGAGACTCTAAATCCAGTGACAAGTCTG |
| TC15P1_pool1_26 | 630 | GAGATCATGTCCTAACCCTGATCCTCTTGTCCCACAGATATCCAGAACCCTCTAAGAAGTTCCTGCTTATTTATAACTCGCTGA |
| | | ATGTGGTTAGAGACATGACCCTGCCGTGTACCAGCTGAGAGACTCTAAATCCAGTGACAAGTCTG |
| TC15P1_pool1_27 | 631 | GAGATCATGTCCTAACCCTGATCCTCTTGTCCCACAGATATCCAGAACCCTCTAAGAAGTTCCTGCTTTTAATTAACTCGGTGA |
| | | ATGTGGTTAGAGACATGACCCTGCCGTGTACCAGCTGAGAGACTCTAAATCCAGTGACAAGTCTG |
| TC15P1_pool1_28 | 632 | GAGATCATGTCCTAACCCTGATCCTCTTGTCCCACAGATATCCAGAACCCTCTAAGAAGTTCCTGCTTTAATATAACTCGTTGAA |
| | | TGTGGTTAGAGACATGACCCTGCCGTGTACCAGCTGAGAGACTCTAAATCCAGTGACAAGTCTG |
| TC15P1_pool1_29 | 633 | GAGATCATGTCCTAACCCTGATCCTCTTGTCCCACAGATATCCAGAACCCTCTAAGAAGTTCCTGCTAAATTTAAACTCTAAGAA |
| | | TGTGGTTAGAGACATGACCCTGCCGTGTACCAGCTGAGAGACTCTAAATCCAGTGACAAGTCTG |
| TC15P1_pool1_30 | 634 | GAGATCATGTCCTAACCCTGATCCTCTTGTCCCACAGATATCCAGAACCCTCTAAGAAGTTCCTGCTATAAAAAAACTCTCTGA |
| | | ATGTGGTTAGAGACATGACCCTGCCGTGTACCAGCTGAGAGACTCTAAATCCAGTGACAAGTCTG |
| TC15P1_pool1_31 | 635 | GAGATCATGTCCTAACCCTGATCCTCTTGTCCCACAGATATCCAGAACCCTCTAAGAAGTTCCTGCTTTAATAATACTCTGAGA |
| | | ATGTGGTTAGAGACATGACCCTGCCGTGTACCAGCTGAGAGACTCTAAATCCAGTGACAAGTCTG |
| TC15P1_pool1_32 | 636 | GAGATCATGTCCTAACCCTGATCCTCTTGTCCCACAGATATCCAGAACCCTCTAAGAAGTTCCTGCTATTAATAAACTCTTTGAA |
| | | TGTGGTTAGAGACATGACCCTGCCGTGTACCAGCTGAGAGACTCTAAATCCAGTGACAAGTCTG |
| TC15P1_pool1_33 | 637 | GAGATCATGTCCTAACCCTGATCCTCTTGTCCCACAGATATCCAGAACCCTCTAAGAAGTTCCTGCTATATTAATAGTCAAAGA |
| | | ATGTGGTTAGAGACATGACCCTGCCGTGTACCAGCTGAGAGACTCTAAATCCAGTGACAAGTCTG |
| TC15P1_pool1_34 | 638 | GAGATCATGTCCTAACCCTGATCCTCTTGTCCCACAGATATCCAGAACCCTCTAAGAAGTTCCTGCTTTATAATTAGTCACTGAA |
| | | TGTGGTTAGAGACATGACCCTGCCGTGTACCAGCTGAGAGACTCTAAATCCAGTGACAAGTCTG |
| TC15P1_pool1_35 | 639 | GAGATCATGTCCTAACCCTGATCCTCTTGTCCCACAGATATCCAGAACCCTCTAAGAAGTTCCTGCTTATATATTAGTCAGTGA |
| | | ATGTGGTTAGAGACATGACCCTGCCGTGTACCAGCTGAGAGACTCTAAATCCAGTGACAAGTCTG |
| TC15P1_pool1_36 | 640 | GAGATCATGTCCTAACCCTGATCCTCTTGTCCCACAGATATCCAGAACCCTCTAAGAAGTTCCTGCTTATATTTTAGTCATAGAA |
| | | TGTGGTTAGAGACATGACCCTGCCGTGTACCAGCTGAGAGACTCTAAATCCAGTGACAAGTCTG |
| TC15P1_pool1_37 | 641 | GAGATCATGTCCTAACCCTGATCCTCTTGTCCCACAGATATCCAGAACCCTCTAAGAAGTTCCTGCTTAATATTAAGTCCATGA |
| | | ATGTGGTTAGAGACATGACCCTGCCGTGTACCAGCTGAGAGACTCTAAATCCAGTGACAAGTCTG |
| TC15P1_pool1_38 | 642 | GAGATCATGTCCTAACCCTGATCCTCTTGTCCCACAGATATCCAGAACCCTCTAAGAAGTTCCTGCTTAAAATTTAGTCCCAGA |
| | | ATGTGGTTAGAGACATGACCCTGCCGTGTACCAGCTGAGAGACTCTAAATCCAGTGACAAGTCTG |
| TC15P1_pool1_39 | 643 | GAGATCATGTCCTAACCCTGATCCTCTTGTCCCACAGATATCCAGAACCCTCTAAGAAGTTCCTGCTATATAATAAGTCCGTGA |
| | | ATGTGGTTAGAGACATGACCCTGCCGTGTACCAGCTGAGAGACTCTAAATCCAGTGACAAGTCTG |
| TC15P1_pool1_40 | 644 | GAGATCATGTCCTAACCCTGATCCTCTTGTCCCACAGATATCCAGAACCCTCTAAGAAGTTCCTGCTAATTAAAAAGTCCTAGA |
| | | ATGTGGTTAGAGACATGACCCTGCCGTGTACCAGCTGAGAGACTCTAAATCCAGTGACAAGTCTG |
| TC15P1_pool1_41 | 645 | GAGATCATGTCCTAACCCTGATCCTCTTGTCCCACAGATATCCAGAACCCTCTAAGAAGTTCCTGCTAATAAAATAGTCGATGA |
| | | ATGTGGTTAGAGACATGACCCTGCCGTGTACCAGCTGAGAGACTCTAAATCCAGTGACAAGTCTG |
| TC15P1_pool1_42 | 646 | GAGATCATGTCCTAACCCTGATCCTCTTGTCCCACAGATATCCAGAACCCTCTAAGAAGTTCCTGCTAAAAATAAAGTCGCTGA |
| | | ATGTGGTTAGAGACATGACCCTGCCGTGTACCAGCTGAGAGACTCTAAATCCAGTGACAAGTCTG |
| TC15P1_pool1_43 | 647 | GAGATCATGTCCTAACCCTGATCCTCTTGTCCCACAGATATCCAGAACCCTCTAAGAAGTTCCTGCTATTTTTAAAGTCGGTGA |
| | | ATGTGGTTAGAGACATGACCCTGCCGTGTACCAGCTGAGAGACTCTAAATCCAGTGACAAGTCTG |
| TC15P1_pool1_44 | 648 | GAGATCATGTCCTAACCCTGATCCTCTTGTCCCACAGATATCCAGAACCCTCTAAGAAGTTCCTGCTTTTTTAAAAGTCGTTGAA |
| | | TGTGGTTAGAGACATGACCCTGCCGTGTACCAGCTGAGAGACTCTAAATCCAGTGACAAGTCTG |
| TC15P1_pool1_45 | 649 | GAGATCATGTCCTAACCCTGATCCTCTTGTCCCACAGATATCCAGAACCCTCTAAGAAGTTCCTGCTATAATTATAGTCTAAGAA |
| | | TGTGGTTAGAGACATGACCCTGCCGTGTACCAGCTGAGAGACTCTAAATCCAGTGACAAGTCTG |
| TC15P1_pool1_46 | 650 | GAGATCATGTCCTAACCCTGATCCTCTTGTCCCACAGATATCCAGAACCCTCTAAGAAGTTCCTGCTAATAAATAAGTCTCAGA |
| | | ATGTGGTTAGAGACATGACCCTGCCGTGTACCAGCTGAGAGACTCTAAATCCAGTGACAAGTCTG |
| TC15P1_pool1_47 | 651 | GAGATCATGTCCTAACCCTGATCCTCTTGTCCCACAGATATCCAGAACCCTCTAAGAAGTTCCTGCTTATATAATAGTCTGAGA |
| | | ATGTGGTTAGAGACATGACCCTGCCGTGTACCAGCTGAGAGACTCTAAATCCAGTGACAAGTCTG |
| TC15P1_pool1_48 | 652 | GAGATCATGTCCTAACCCTGATCCTCTTGTCCCACAGATATCCAGAACCCTCTAAGAAGTTCCTGCTTAAAAAAAAGTCTTTGA |
| | | ATGTGGTTAGAGACATGACCCTGCCGTGTACCAGCTGAGAGACTCTAAATCCAGTGACAAGTCTG |
| TC15P1_pool1_49 | 653 | GAGATCATGTCCTAACCCTGATCCTCTTGTCCCACAGATATCCAGAACCCTCTAAGAAGTTCCTGCTATTTAATTATTCAAAGAA |
| | | TGTGGTTAGAGACATGACCCTGCCGTGTACCAGCTGAGAGACTCTAAATCCAGTGACAAGTCTG |
| TC15P1_pool1_50 | 654 | GAGATCATGTCCTAACCCTGATCCTCTTGTCCCACAGATATCCAGAACCCTCTAAGAAGTTCCTGCTTTATAATAATTCACAGAA |
| | | TGTGGTTAGAGACATGACCCTGCCGTGTACCAGCTGAGAGACTCTAAATCCAGTGACAAGTCTG |
| TC15P1_pool1_51 | 655 | GAGATCATGTCCTAACCCTGATCCTCTTGTCCCACAGATATCCAGAACCCTCTAAGAAGTTCCTGCTTATATATAATTCAGAGAA |
| | | TGTGGTTAGAGACATGACCCTGCCGTGTACCAGCTGAGAGACTCTAAATCCAGTGACAAGTCTG |
| TC15P1_pool1_52 | 656 | GAGATCATGTCCTAACCCTGATCCTCTTGTCCCACAGATATCCAGAACCCTCTAAGAAGTTCCTGCTAAAAAAAAATTCATAGA |
| | | ATGTGGTTAGAGACATGACCCTGCCGTGTACCAGCTGAGAGACTCTAAATCCAGTGACAAGTCTG |
| TC15P1_pool1_53 | 657 | GAGATCATGTCCTAACCCTGATCCTCTTGTCCCACAGATATCCAGAACCCTCTAAGAAGTTCCTGCTTATTTTAAATTCCAAGAA |
| | | TGTGGTTAGAGACATGACCCTGCCGTGTACCAGCTGAGAGACTCTAAATCCAGTGACAAGTCTG |
| TC15P1_pool1_54 | 658 | GAGATCATGTCCTAACCCTGATCCTCTTGTCCCACAGATATCCAGAACCCTCTAAGAAGTTCCTGCTAATTTTTAATTCCCAGAA |
| | | TGTGGTTAGAGACATGACCCTGCCGTGTACCAGCTGAGAGACTCTAAATCCAGTGACAAGTCTG |
| TC15P1_pool1_55 | 659 | GAGATCATGTCCTAACCCTGATCCTCTTGTCCCACAGATATCCAGAACCCTCTAAGAAGTTCCTGCTAAATTATAATTCCGAGA |
| | | ATGTGGTTAGAGACATGACCCTGCCGTGTACCAGCTGAGAGACTCTAAATCCAGTGACAAGTCTG |
| TC15P1_pool1_56 | 660 | GAGATCATGTCCTAACCCTGATCCTCTTGTCCCACAGATATCCAGAACCCTCTAAGAAGTTCCTGCTTTTAAATTATTCCTTGAA |
| | | TGTGGTTAGAGACATGACCCTGCCGTGTACCAGCTGAGAGACTCTAAATCCAGTGACAAGTCTG |
| TC15P1_pool1_57 | 661 | GAGATCATGTCCTAACCCTGATCCTCTTGTCCCACAGATATCCAGAACCCTCTAAGAAGTTCCTGCTTTTAATTTATTCGAAGAA |
| | | TGTGGTTAGAGACATGACCCTGCCGTGTACCAGCTGAGAGACTCTAAATCCAGTGACAAGTCTG |
| TC15P1_pool1_58 | 662 | GAGATCATGTCCTAACCCTGATCCTCTTGTCCCACAGATATCCAGAACCCTCTAAGAAGTTCCTGCTATAATTAAATTCGCTGA |
| | | ATGTGGTTAGAGACATGACCCTGCCGTGTACCAGCTGAGAGACTCTAAATCCAGTGACAAGTCTG |
| TC15P1_pool1_59 | 663 | GAGATCATGTCCTAACCCTGATCCTCTTGTCCCACAGATATCCAGAACCCTCTAAGAAGTTCCTGCTTTAAAATTATTCGGAGA |
| | | ATGTGGTTAGAGACATGACCCTGCCGTGTACCAGCTGAGAGACTCTAAATCCAGTGACAAGTCTG |
| TC15P1_pool1_60 | 664 | GAGATCATGTCCTAACCCTGATCCTCTTGTCCCACAGATATCCAGAACCCTCTAAGAAGTTCCTGCTTTATTTTTATTCGTTGAA |
| | | TGTGGTTAGAGACATGACCCTGCCGTGTACCAGCTGAGAGACTCTAAATCCAGTGACAAGTCTG |
| TC15P1_pool1_61 | 665 | GAGATCATGTCCTAACCCTGATCCTCTTGTCCCACAGATATCCAGAACCCTCTAAGAAGTTCCTGCTTAATTAAAATTCTATGAA |
| | | TGTGGTTAGAGACATGACCCTGCCGTGTACCAGCTGAGAGACTCTAAATCCAGTGACAAGTCTG |
| TC15P1_pool1_62 | 666 | GAGATCATGTCCTAACCCTGATCCTCTTGTCCCACAGATATCCAGAACCCTCTAAGAAGTTCCTGCTTATAAAAAATTCTCAGAA |
| | | TGTGGTTAGAGACATGACCCTGCCGTGTACCAGCTGAGAGACTCTAAATCCAGTGACAAGTCTG |
| TC15P1_pool1_63 | 667 | GAGATCATGTCCTAACCCTGATCCTCTTGTCCCACAGATATCCAGAACCCTCTAAGAAGTTCCTGCTTATATTTAATTCTGTGAA |
| | | TGTGGTTAGAGACATGACCCTGCCGTGTACCAGCTGAGAGACTCTAAATCCAGTGACAAGTCTG |
| TC15P1_pool1_64 | 668 | GAGATCATGTCCTAACCCTGATCCTCTTGTCCCACAGATATCCAGAACCCTCTAAGAAGTTCCTGCTTAAATAATATTCTTTGAA |
| | | TGTGGTTAGAGACATGACCCTGCCGTGTACCAGCTGAGAGACTCTAAATCCAGTGACAAGTCTG |
| TC15P1_pool1_65 | 669 | GAGATCATGTCCTAACCCTGATCCTCTTGTCCCACAGATATCCAGAACCCTCTAAGAAGTTCCTGCTAATTTTATCATCAAAGAA |
| | | TGTGGTTAGAGACATGACCCTGCCGTGTACCAGCTGAGAGACTCTAAATCCAGTGACAAGTCTG |
| TC15P1_pool1_66 | 670 | GAGATCATGTCCTAACCCTGATCCTCTTGTCCCACAGATATCCAGAACCCTCTAAGAAGTTCCTGCTATTTATTACATCACTGAA |
| | | TGTGGTTAGAGACATGACCCTGCCGTGTACCAGCTGAGAGACTCTAAATCCAGTGACAAGTCTG |
| TC15P1_pool1_67 | 671 | GAGATCATGTCCTAACCCTGATCCTCTTGTCCCACAGATATCCAGAACCCTCTAAGAAGTTCCTGCTAATTTTTTCATCAGTGAA |
| | | TGTGGTTAGAGACATGACCCTGCCGTGTACCAGCTGAGAGACTCTAAATCCAGTGACAAGTCTG |
| TC15P1_pool1_68 | 672 | GAGATCATGTCCTAACCCTGATCCTCTTGTCCCACAGATATCCAGAACCCTCTAAGAAGTTCCTGCTTTTTAATACATCATTGAA |
| | | TGTGGTTAGAGACATGACCCTGCCGTGTACCAGCTGAGAGACTCTAAATCCAGTGACAAGTCTG |
| TC15P1_pool1_69 | 673 | GAGATCATGTCCTAACCCTGATCCTCTTGTCCCACAGATATCCAGAACCCTCTAAGAAGTTCCTGCTAAATTTTACATCCATGAA |
| | | TGTGGTTAGAGACATGACCCTGCCGTGTACCAGCTGAGAGACTCTAAATCCAGTGACAAGTCTG |
| TC15P1_pool1_70 | 674 | GAGATCATGTCCTAACCCTGATCCTCTTGTCCCACAGATATCCAGAACCCTCTAAGAAGTTCCTGCTTTTTAAATCATCCCTGAA |
| | | TGTGGTTAGAGACATGACCCTGCCGTGTACCAGCTGAGAGACTCTAAATCCAGTGACAAGTCTG |
| TC15P1_pool1_71 | 675 | GAGATCATGTCCTAACCCTGATCCTCTTGTCCCACAGATATCCAGAACCCTCTAAGAAGTTCCTGCTATATTTTTCATCCGAGA |
| | | ATGTGGTTAGAGACATGACCCTGCCGTGTACCAGCTGAGAGACTCTAAATCCAGTGACAAGTCTG |
| TC15P1_pool1_72 | 676 | GAGATCATGTCCTAACCCTGATCCTCTTGTCCCACAGATATCCAGAACCCTCTAAGAAGTTCCTGCTTATAAATTCATCCTAGAA |
| | | TGTGGTTAGAGACATGACCCTGCCGTGTACCAGCTGAGAGACTCTAAATCCAGTGACAAGTCTG |
| TC15P1_pool1_73 | 677 | GAGATCATGTCCTAACCCTGATCCTCTTGTCCCACAGATATCCAGAACCCTCTAAGAAGTTCCTGCTAATATTAACATCGATGA |
| | | ATGTGGTTAGAGACATGACCCTGCCGTGTACCAGCTGAGAGACTCTAAATCCAGTGACAAGTCTG |
| TC15P1_pool1_74 | 678 | GAGATCATGTCCTAACCCTGATCCTCTTGTCCCACAGATATCCAGAACCCTCTAAGAAGTTCCTGCTAAATTTATCATCGCTGA |
| | | ATGTGGTTAGAGACATGACCCTGCCGTGTACCAGCTGAGAGACTCTAAATCCAGTGACAAGTCTG |
| TC15P1_pool1_75 | 679 | GAGATCATGTCCTAACCCTGATCCTCTTGTCCCACAGATATCCAGAACCCTCTAAGAAGTTCCTGCTTTATAAAACATCGGTGA |
| | | ATGTGGTTAGAGACATGACCCTGCCGTGTACCAGCTGAGAGACTCTAAATCCAGTGACAAGTCTG |
| TC15P1_pool1_76 | 680 | GAGATCATGTCCTAACCCTGATCCTCTTGTCCCACAGATATCCAGAACCCTCTAAGAAGTTCCTGCTTAAAAATACATCGTAGA |
| | | ATGTGGTTAGAGACATGACCCTGCCGTGTACCAGCTGAGAGACTCTAAATCCAGTGACAAGTCTG |
| TC15P1_pool1_77 | 681 | GAGATCATGTCCTAACCCTGATCCTCTTGTCCCACAGATATCCAGAACCCTCTAAGAAGTTCCTGCTATTAATTTCATCTATGAA |
| | | TGTGGTTAGAGACATGACCCTGCCGTGTACCAGCTGAGAGACTCTAAATCCAGTGACAAGTCTG |
| TC15P1_pool1_78 | 682 | GAGATCATGTCCTAACCCTGATCCTCTTGTCCCACAGATATCCAGAACCCTCTAAGAAGTTCCTGCTTATATAAACATCTCTGAA |
| | | TGTGGTTAGAGACATGACCCTGCCGTGTACCAGCTGAGAGACTCTAAATCCAGTGACAAGTCTG |
| TC15P1_pool1_79 | 683 | GAGATCATGTCCTAACCCTGATCCTCTTGTCCCACAGATATCCAGAACCCTCTAAGAAGTTCCTGCTATAAATTACATCTGTGA |
| | | ATGTGGTTAGAGACATGACCCTGCCGTGTACCAGCTGAGAGACTCTAAATCCAGTGACAAGTCTG |
| TC15P1_pool1_80 | 684 | GAGATCATGTCCTAACCCTGATCCTCTTGTCCCACAGATATCCAGAACCCTCTAAGAAGTTCCTGCTTTTATTATCATCTTTGAA |
| | | TGTGGTTAGAGACATGACCCTGCCGTGTACCAGCTGAGAGACTCTAAATCCAGTGACAAGTCTG |
| TC15P1_pool1_81 | 685 | GAGATCATGTCCTAACCCTGATCCTCTTGTCCCACAGATATCCAGAACCCTCTAAGAAGTTCCTGCTATAAATTTCCTCAAAGA |
| | | ATGTGGTTAGAGACATGACCCTGCCGTGTACCAGCTGAGAGACTCTAAATCCAGTGACAAGTCTG |
| TC15P1_pool1_82 | 686 | GAGATCATGTCCTAACCCTGATCCTCTTGTCCCACAGATATCCAGAACCCTCTAAGAAGTTCCTGCTATTATTTACCTCACTGAA |
| | | TGTGGTTAGAGACATGACCCTGCCGTGTACCAGCTGAGAGACTCTAAATCCAGTGACAAGTCTG |
| TC15P1_pool1_83 | 687 | GAGATCATGTCCTAACCCTGATCCTCTTGTCCCACAGATATCCAGAACCCTCTAAGAAGTTCCTGCTAAATAAAACCTCAGTGA |
| | | ATGTGGTTAGAGACATGACCCTGCCGTGTACCAGCTGAGAGACTCTAAATCCAGTGACAAGTCTG |
| TC15P1_pool1_84 | 688 | GAGATCATGTCCTAACCCTGATCCTCTTGTCCCACAGATATCCAGAACCCTCTAAGAAGTTCCTGCTTAATTAATCCTCATAGAA |
| | | TGTGGTTAGAGACATGACCCTGCCGTGTACCAGCTGAGAGACTCTAAATCCAGTGACAAGTCTG |
| TC15P1_pool1_85 | 689 | GAGATCATGTCCTAACCCTGATCCTCTTGTCCCACAGATATCCAGAACCCTCTAAGAAGTTCCTGCTAATTATAACCTCCATGA |
| | | ATGTGGTTAGAGACATGACCCTGCCGTGTACCAGCTGAGAGACTCTAAATCCAGTGACAAGTCTG |
| TC15P1_pool1_86 | 690 | GAGATCATGTCCTAACCCTGATCCTCTTGTCCCACAGATATCCAGAACCCTCTAAGAAGTTCCTGCTATATTATTCCTCCCTGA |
| | | ATGTGGTTAGAGACATGACCCTGCCGTGTACCAGCTGAGAGACTCTAAATCCAGTGACAAGTCTG |
| TC15P1_pool1_87 | 691 | GAGATCATGTCCTAACCCTGATCCTCTTGTCCCACAGATATCCAGAACCCTCTAAGAAGTTCCTGCTTTATAAATCCTCCGAGA |
| | | ATGTGGTTAGAGACATGACCCTGCCGTGTACCAGCTGAGAGACTCTAAATCCAGTGACAAGTCTG |
| TC15P1_pool1_88 | 692 | GAGATCATGTCCTAACCCTGATCCTCTTGTCCCACAGATATCCAGAACCCTCTAAGAAGTTCCTGCTTTTTATATCCTCCTAGAA |
| | | TGTGGTTAGAGACATGACCCTGCCGTGTACCAGCTGAGAGACTCTAAATCCAGTGACAAGTCTG |
| TC15P1_pool1_89 | 693 | GAGATCATGTCCTAACCCTGATCCTCTTGTCCCACAGATATCCAGAACCCTCTAAGAAGTTCCTGCTAAAATTTTCCTCGAAGA |
| | | ATGTGGTTAGAGACATGACCCTGCCGTGTACCAGCTGAGAGACTCTAAATCCAGTGACAAGTCTG |
| TC15P1_pool1_90 | 694 | GAGATCATGTCCTAACCCTGATCCTCTTGTCCCACAGATATCCAGAACCCTCTAAGAAGTTCCTGCTTATTAAAACCTCGCTGA |
| | | ATGTGGTTAGAGACATGACCCTGCCGTGTACCAGCTGAGAGACTCTAAATCCAGTGACAAGTCTG |
| TC15P1_pool1_91 | 695 | GAGATCATGTCCTAACCCTGATCCTCTTGTCCCACAGATATCCAGAACCCTCTAAGAAGTTCCTGCTTAAATTAACCTCGGAGA |
| | | ATGTGGTTAGAGACATGACCCTGCCGTGTACCAGCTGAGAGACTCTAAATCCAGTGACAAGTCTG |
| TC15P1_pool1_92 | 696 | GAGATCATGTCCTAACCCTGATCCTCTTGTCCCACAGATATCCAGAACCCTCTAAGAAGTTCCTGCTTAAATATTCCTCGTAGA |
| | | ATGTGGTTAGAGACATGACCCTGCCGTGTACCAGCTGAGAGACTCTAAATCCAGTGACAAGTCTG |
| TC15P1_pool1_93 | 697 | GAGATCATGTCCTAACCCTGATCCTCTTGTCCCACAGATATCCAGAACCCTCTAAGAAGTTCCTGCTAATAATTACCTCTATGAA |
| | | TGTGGTTAGAGACATGACCCTGCCGTGTACCAGCTGAGAGACTCTAAATCCAGTGACAAGTCTG |
| TC15P1_pool1_94 | 698 | GAGATCATGTCCTAACCCTGATCCTCTTGTCCCACAGATATCCAGAACCCTCTAAGAAGTTCCTGCTATATAAATCCTCTCTGA |
| | | ATGTGGTTAGAGACATGACCCTGCCGTGTACCAGCTGAGAGACTCTAAATCCAGTGACAAGTCTG |
| TC15P1_pool1_95 | 699 | GAGATCATGTCCTAACCCTGATCCTCTTGTCCCACAGATATCCAGAACCCTCTAAGAAGTTCCTGCTTTAAATATCCTCTGTGA |
| | | ATGTGGTTAGAGACATGACCCTGCCGTGTACCAGCTGAGAGACTCTAAATCCAGTGACAAGTCTG |
| TC15P1_pool1_96 | 700 | GAGATCATGTCCTAACCCTGATCCTCTTGTCCCACAGATATCCAGAACCCTCTAAGAAGTTCCTGCTAAAAATATCCTCTTAGA |
| | | ATGTGGTTAGAGACATGACCCTGCCGTGTACCAGCTGAGAGACTCTAAATCCAGTGACAAGTCTG |
| TC15P2_pool1_97 | 701 | GAGATCATGTCCTAACCCTGATCCTCTTGTCCCACAGATATCCAGAACCCTCTAAGAAGTTCCTGCTATATATATCGTCAAAGA |
| | | ATGTGGTTAGAGACATGACCCTGCCGTGTACCAGCTGAGAGACTCTAAATCCAGTGACAAGTCTG |
| TC15P2_pool1_98 | 702 | GAGATCATGTCCTAACCCTGATCCTCTTGTCCCACAGATATCCAGAACCCTCTAAGAAGTTCCTGCTTTATTAATCGTCACTGA |
| | | ATGTGGTTAGAGACATGACCCTGCCGTGTACCAGCTGAGAGACTCTAAATCCAGTGACAAGTCTG |
| TC15P2_pool1_99 | 703 | GAGATCATGTCCTAACCCTGATCCTCTTGTCCCACAGATATCCAGAACCCTCTAAGAAGTTCCTGCTATTATAAACGTCAGAGA |
| | | ATGTGGTTAGAGACATGACCCTGCCGTGTACCAGCTGAGAGACTCTAAATCCAGTGACAAGTCTG |
| TC15P2_pool1_100 | 704 | GAGATCATGTCCTAACCCTGATCCTCTTGTCCCACAGATATCCAGAACCCTCTAAGAAGTTCCTGCTTTTAAAAACGTCATTGA |
| | | ATGTGGTTAGAGACATGACCCTGCCGTGTACCAGCTGAGAGACTCTAAATCCAGTGACAAGTCTG |
| TC15P2_pool1_101 | 705 | GAGATCATGTCCTAACCCTGATCCTCTTGTCCCACAGATATCCAGAACCCTCTAAGAAGTTCCTGCTTATAAATACGTCCATGA |
| | | ATGTGGTTAGAGACATGACCCTGCCGTGTACCAGCTGAGAGACTCTAAATCCAGTGACAAGTCTG |
| TC15P2_pool1_102 | 706 | GAGATCATGTCCTAACCCTGATCCTCTTGTCCCACAGATATCCAGAACCCTCTAAGAAGTTCCTGCTTTTAATAACGTCCCAGA |
| | | ATGTGGTTAGAGACATGACCCTGCCGTGTACCAGCTGAGAGACTCTAAATCCAGTGACAAGTCTG |
| TC15P2_pool1_103 | 707 | GAGATCATGTCCTAACCCTGATCCTCTTGTCCCACAGATATCCAGAACCCTCTAAGAAGTTCCTGCTATAATTTTCGTCCGTGA |
| | | ATGTGGTTAGAGACATGACCCTGCCGTGTACCAGCTGAGAGACTCTAAATCCAGTGACAAGTCTG |
| TC15P2_pool1_104 | 708 | GAGATCATGTCCTAACCCTGATCCTCTTGTCCCACAGATATCCAGAACCCTCTAAGAAGTTCCTGCTTTTTTATTCGTCCTAGAA |
| | | TGTGGTTAGAGACATGACCCTGCCGTGTACCAGCTGAGAGACTCTAAATCCAGTGACAAGTCTG |
| TC15P2_pool1_105 | 709 | GAGATCATGTCCTAACCCTGATCCTCTTGTCCCACAGATATCCAGAACCCTCTAAGAAGTTCCTGCTTAATTATTCGTCGATGA |
| | | ATGTGGTTAGAGACATGACCCTGCCGTGTACCAGCTGAGAGACTCTAAATCCAGTGACAAGTCTG |
| TC15P2_pool1_106 | 710 | GAGATCATGTCCTAACCCTGATCCTCTTGTCCCACAGATATCCAGAACCCTCTAAGAAGTTCCTGCTATAAAAATCGTCGCAGA |
| | | ATGTGGTTAGAGACATGACCCTGCCGTGTACCAGCTGAGAGACTCTAAATCCAGTGACAAGTCTG |
| TC15P2_pool1_107 | 711 | GAGATCATGTCCTAACCCTGATCCTCTTGTCCCACAGATATCCAGAACCCTCTAAGAAGTTCCTGCTTATAATATCGTCGGAGA |
| | | ATGTGGTTAGAGACATGACCCTGCCGTGTACCAGCTGAGAGACTCTAAATCCAGTGACAAGTCTG |
| TC15P2_pool1_108 | 712 | GAGATCATGTCCTAACCCTGATCCTCTTGTCCCACAGATATCCAGAACCCTCTAAGAAGTTCCTGCTTAATATATCGTCGTAGA |
| | | ATGTGGTTAGAGACATGACCCTGCCGTGTACCAGCTGAGAGACTCTAAATCCAGTGACAAGTCTG |
| TC15P2_pool1_109 | 713 | GAGATCATGTCCTAACCCTGATCCTCTTGTCCCACAGATATCCAGAACCCTCTAAGAAGTTCCTGCTTAATTTTTCGTCTAAGAA |
| | | TGTGGTTAGAGACATGACCCTGCCGTGTACCAGCTGAGAGACTCTAAATCCAGTGACAAGTCTG |
| TC15P2_pool1_110 | 714 | GAGATCATGTCCTAACCCTGATCCTCTTGTCCCACAGATATCCAGAACCCTCTAAGAAGTTCCTGCTATTTAAAACGTCTCTGA |
| | | ATGTGGTTAGAGACATGACCCTGCCGTGTACCAGCTGAGAGACTCTAAATCCAGTGACAAGTCTG |
| TC15P2_pool1_111 | 715 | GAGATCATGTCCTAACCCTGATCCTCTTGTCCCACAGATATCCAGAACCCTCTAAGAAGTTCCTGCTATATAAAACGTCTGAGA |
| | | ATGTGGTTAGAGACATGACCCTGCCGTGTACCAGCTGAGAGACTCTAAATCCAGTGACAAGTCTG |
| TC15P2_pool1_112 | 716 | GAGATCATGTCCTAACCCTGATCCTCTTGTCCCACAGATATCCAGAACCCTCTAAGAAGTTCCTGCTATAATATTCGTCTTAGAA |
| | | TGTGGTTAGAGACATGACCCTGCCGTGTACCAGCTGAGAGACTCTAAATCCAGTGACAAGTCTG |
| TC15P2_pool1_113 | 717 | GAGATCATGTCCTAACCCTGATCCTCTTGTCCCACAGATATCCAGAACCCTCTAAGAAGTTCCTGCTATTATTTTCTTCAAAGAA |
| | | TGTGGTTAGAGACATGACCCTGCCGTGTACCAGCTGAGAGACTCTAAATCCAGTGACAAGTCTG |
| TC15P2_pool1_114 | 718 | GAGATCATGTCCTAACCCTGATCCTCTTGTCCCACAGATATCCAGAACCCTCTAAGAAGTTCCTGCTAATTAATACTTCACTGAA |
| | | TGTGGTTAGAGACATGACCCTGCCGTGTACCAGCTGAGAGACTCTAAATCCAGTGACAAGTCTG |
| TC15P2_pool1_115 | 719 | GAGATCATGTCCTAACCCTGATCCTCTTGTCCCACAGATATCCAGAACCCTCTAAGAAGTTCCTGCTTATTATTACTTCAGAGAA |
| | | TGTGGTTAGAGACATGACCCTGCCGTGTACCAGCTGAGAGACTCTAAATCCAGTGACAAGTCTG |
| TC15P2_pool1_116 | 720 | GAGATCATGTCCTAACCCTGATCCTCTTGTCCCACAGATATCCAGAACCCTCTAAGAAGTTCCTGCTATTAAATACTTCATTGAA |
| | | TGTGGTTAGAGACATGACCCTGCCGTGTACCAGCTGAGAGACTCTAAATCCAGTGACAAGTCTG |
| TC15P2_pool1_117 | 721 | GAGATCATGTCCTAACCCTGATCCTCTTGTCCCACAGATATCCAGAACCCTCTAAGAAGTTCCTGCTAAAAAATTCTTCCATGA |
| | | ATGTGGTTAGAGACATGACCCTGCCGTGTACCAGCTGAGAGACTCTAAATCCAGTGACAAGTCTG |
| TC15P2_pool1_118 | 722 | GAGATCATGTCCTAACCCTGATCCTCTTGTCCCACAGATATCCAGAACCCTCTAAGAAGTTCCTGCTTTTTAAAACTTCCCAGA |
| | | ATGTGGTTAGAGACATGACCCTGCCGTGTACCAGCTGAGAGACTCTAAATCCAGTGACAAGTCTG |
| TC15P2_pool1_119 | 723 | GAGATCATGTCCTAACCCTGATCCTCTTGTCCCACAGATATCCAGAACCCTCTAAGAAGTTCCTGCTTAAATTTTCTTCCGTGAA |
| | | TGTGGTTAGAGACATGACCCTGCCGTGTACCAGCTGAGAGACTCTAAATCCAGTGACAAGTCTG |
| TC15P2_pool1_120 | 724 | GAGATCATGTCCTAACCCTGATCCTCTTGTCCCACAGATATCCAGAACCCTCTAAGAAGTTCCTGCTTTATTTATCTTCCTAGAA |
| | | TGTGGTTAGAGACATGACCCTGCCGTGTACCAGCTGAGAGACTCTAAATCCAGTGACAAGTCTG |
| TC15P2_pool1_121 | 725 | GAGATCATGTCCTAACCCTGATCCTCTTGTCCCACAGATATCCAGAACCCTCTAAGAAGTTCCTGCTATAAAATACTTCGAAGA |
| | | ATGTGGTTAGAGACATGACCCTGCCGTGTACCAGCTGAGAGACTCTAAATCCAGTGACAAGTCTG |
| TC15P2_pool1_122 | 726 | GAGATCATGTCCTAACCCTGATCCTCTTGTCCCACAGATATCCAGAACCCTCTAAGAAGTTCCTGCTTTTTTTTACTTCGCAGAA |
| | | TGTGGTTAGAGACATGACCCTGCCGTGTACCAGCTGAGAGACTCTAAATCCAGTGACAAGTCTG |
| TC15P2_pool1_123 | 727 | GAGATCATGTCCTAACCCTGATCCTCTTGTCCCACAGATATCCAGAACCCTCTAAGAAGTTCCTGCTTATTAAATCTTCGGAGA |
| | | ATGTGGTTAGAGACATGACCCTGCCGTGTACCAGCTGAGAGACTCTAAATCCAGTGACAAGTCTG |
| TC15P2_pool1_124 | 728 | GAGATCATGTCCTAACCCTGATCCTCTTGTCCCACAGATATCCAGAACCCTCTAAGAAGTTCCTGCTATTAATTACTTCGTAGAA |
| | | TGTGGTTAGAGACATGACCCTGCCGTGTACCAGCTGAGAGACTCTAAATCCAGTGACAAGTCTG |
| TC15P2_pool1_125 | 729 | GAGATCATGTCCTAACCCTGATCCTCTTGTCCCACAGATATCCAGAACCCTCTAAGAAGTTCCTGCTAAATATTACTTCTAAGAA |
| | | TGTGGTTAGAGACATGACCCTGCCGTGTACCAGCTGAGAGACTCTAAATCCAGTGACAAGTCTG |
| TC15P2_pool2_1 | 730 | GAGATCATGTCCTAACCCTGATCCTCTTGTCCCACAGATATCCAGAACCCTCTAAGAAGTTCCTGCTTTTAAAATCTTCTCAGAA |
| | | TGTGGTTAGAGACATGACCCTGCCGTGTACCAGCTGAGAGACTCTAAATCCAGTGACAAGTCTG |
| TC15P2_pool2_2 | 731 | GAGATCATGTCCTAACCCTGATCCTCTTGTCCCACAGATATCCAGAACCCTCTAAGAAGTTCCTGCTATTTTAATCTTCTGTGAA |
| | | TGTGGTTAGAGACATGACCCTGCCGTGTACCAGCTGAGAGACTCTAAATCCAGTGACAAGTCTG |
| TC15P2_pool2_3 | 732 | GAGATCATGTCCTAACCCTGATCCTCTTGTCCCACAGATATCCAGAACCCTCTAAGAAGTTCCTGCTATAAATAACTTCTTAGAA |
| | | TGTGGTTAGAGACATGACCCTGCCGTGTACCAGCTGAGAGACTCTAAATCCAGTGACAAGTCTG |
| TC15P2_pool2_4 | 733 | GAGATCATGTCCTAACCCTGATCCTCTTGTCCCACAGATATCCAGAACCCTCTAAGAAGTTCCTGCTTTATATTTGATCAAAGAA |
| | | TGTGGTTAGAGACATGACCCTGCCGTGTACCAGCTGAGAGACTCTAAATCCAGTGACAAGTCTG |
| TC15P2_pool2_5 | 734 | GAGATCATGTCCTAACCCTGATCCTCTTGTCCCACAGATATCCAGAACCCTCTAAGAAGTTCCTGCTAATATATTGATCACAGA |
| | | ATGTGGTTAGAGACATGACCCTGCCGTGTACCAGCTGAGAGACTCTAAATCCAGTGACAAGTCTG |
| TC15P2_pool2_6 | 735 | GAGATCATGTCCTAACCCTGATCCTCTTGTCCCACAGATATCCAGAACCCTCTAAGAAGTTCCTGCTTTTAAATAGATCAGAGA |
| | | ATGTGGTTAGAGACATGACCCTGCCGTGTACCAGCTGAGAGACTCTAAATCCAGTGACAAGTCTG |
| TC15P2_pool2_7 | 736 | GAGATCATGTCCTAACCCTGATCCTCTTGTCCCACAGATATCCAGAACCCTCTAAGAAGTTCCTGCTTTAAAAAAGATCATAGA |
| | | ATGTGGTTAGAGACATGACCCTGCCGTGTACCAGCTGAGAGACTCTAAATCCAGTGACAAGTCTG |
| TC15P2_pool2_8 | 737 | GAGATCATGTCCTAACCCTGATCCTCTTGTCCCACAGATATCCAGAACCCTCTAAGAAGTTCCTGCTTATTATTTGATCCATGAA |
| | | TGTGGTTAGAGACATGACCCTGCCGTGTACCAGCTGAGAGACTCTAAATCCAGTGACAAGTCTG |
| TC15P2_pool2_9 | 738 | GAGATCATGTCCTAACCCTGATCCTCTTGTCCCACAGATATCCAGAACCCTCTAAGAAGTTCCTGCTTTATTTAAGATCCCTGA |
| | | ATGTGGTTAGAGACATGACCCTGCCGTGTACCAGCTGAGAGACTCTAAATCCAGTGACAAGTCTG |
| TC15P2_pool2_10 | 739 | GAGATCATGTCCTAACCCTGATCCTCTTGTCCCACAGATATCCAGAACCCTCTAAGAAGTTCCTGCTAAAATAATGATCCGAGA |
| | | ATGTGGTTAGAGACATGACCCTGCCGTGTACCAGCTGAGAGACTCTAAATCCAGTGACAAGTCTG |
| TC15P2_pool2_11 | 740 | GAGATCATGTCCTAACCCTGATCCTCTTGTCCCACAGATATCCAGAACCCTCTAAGAAGTTCCTGCTATATTAAAGATCCTTGA |
| | | ATGTGGTTAGAGACATGACCCTGCCGTGTACCAGCTGAGAGACTCTAAATCCAGTGACAAGTCTG |
| TC15P2_pool2_12 | 741 | GAGATCATGTCCTAACCCTGATCCTCTTGTCCCACAGATATCCAGAACCCTCTAAGAAGTTCCTGCTATTAATATGATCGAAGA |
| | | ATGTGGTTAGAGACATGACCCTGCCGTGTACCAGCTGAGAGACTCTAAATCCAGTGACAAGTCTG |
| TC15P2_pool2_13 | 742 | GAGATCATGTCCTAACCCTGATCCTCTTGTCCCACAGATATCCAGAACCCTCTAAGAAGTTCCTGCTATTATATTGATCGCTGA |
| | | ATGTGGTTAGAGACATGACCCTGCCGTGTACCAGCTGAGAGACTCTAAATCCAGTGACAAGTCTG |
| TC15P2_pool2_14 | 743 | GAGATCATGTCCTAACCCTGATCCTCTTGTCCCACAGATATCCAGAACCCTCTAAGAAGTTCCTGCTATATATTTGATCGGTGA |
| | | ATGTGGTTAGAGACATGACCCTGCCGTGTACCAGCTGAGAGACTCTAAATCCAGTGACAAGTCTG |
| TC15P2_pool2_15 | 744 | GAGATCATGTCCTAACCCTGATCCTCTTGTCCCACAGATATCCAGAACCCTCTAAGAAGTTCCTGCTAAAATATAGATCGTTGA |
| | | ATGTGGTTAGAGACATGACCCTGCCGTGTACCAGCTGAGAGACTCTAAATCCAGTGACAAGTCTG |
| TC15P2_pool2_16 | 745 | GAGATCATGTCCTAACCCTGATCCTCTTGTCCCACAGATATCCAGAACCCTCTAAGAAGTTCCTGCTTATAATAAGATCTATGAA |
| | | TGTGGTTAGAGACATGACCCTGCCGTGTACCAGCTGAGAGACTCTAAATCCAGTGACAAGTCTG |
| TC15P2_pool2_17 | 746 | GAGATCATGTCCTAACCCTGATCCTCTTGTCCCACAGATATCCAGAACCCTCTAAGAAGTTCCTGCTTAATAAATGATCTCTGA |
| | | ATGTGGTTAGAGACATGACCCTGCCGTGTACCAGCTGAGAGACTCTAAATCCAGTGACAAGTCTG |
| TC15P2_pool2_18 | 747 | GAGATCATGTCCTAACCCTGATCCTCTTGTCCCACAGATATCCAGAACCCTCTAAGAAGTTCCTGCTAATAATAAGATCTGAGA |
| | | ATGTGGTTAGAGACATGACCCTGCCGTGTACCAGCTGAGAGACTCTAAATCCAGTGACAAGTCTG |
| TC15P2_pool2_19 | 748 | GAGATCATGTCCTAACCCTGATCCTCTTGTCCCACAGATATCCAGAACCCTCTAAGAAGTTCCTGCTATAATAATGATCTTTGAA |
| | | TGTGGTTAGAGACATGACCCTGCCGTGTACCAGCTGAGAGACTCTAAATCCAGTGACAAGTCTG |
| TC15P2_pool2_20 | 749 | GAGATCATGTCCTAACCCTGATCCTCTTGTCCCACAGATATCCAGAACCCTCTAAGAAGTTCCTGCTTTATTAAAGCTCAAAGA |
| | | ATGTGGTTAGAGACATGACCCTGCCGTGTACCAGCTGAGAGACTCTAAATCCAGTGACAAGTCTG |
| TC15P2_pool2_21 | 750 | GAGATCATGTCCTAACCCTGATCCTCTTGTCCCACAGATATCCAGAACCCTCTAAGAAGTTCCTGCTAATTATTTGCTCACAGA |
| | | ATGTGGTTAGAGACATGACCCTGCCGTGTACCAGCTGAGAGACTCTAAATCCAGTGACAAGTCTG |
| TC15P2_pool2_22 | 751 | GAGATCATGTCCTAACCCTGATCCTCTTGTCCCACAGATATCCAGAACCCTCTAAGAAGTTCCTGCTAAATAATTGCTCAGAGA |
| | | ATGTGGTTAGAGACATGACCCTGCCGTGTACCAGCTGAGAGACTCTAAATCCAGTGACAAGTCTG |
| TC15P2_pool2_23 | 752 | GAGATCATGTCCTAACCCTGATCCTCTTGTCCCACAGATATCCAGAACCCTCTAAGAAGTTCCTGCTTATTTAATGCTCATTGAA |
| | | TGTGGTTAGAGACATGACCCTGCCGTGTACCAGCTGAGAGACTCTAAATCCAGTGACAAGTCTG |
| TC15P2_pool2_24 | 753 | GAGATCATGTCCTAACCCTGATCCTCTTGTCCCACAGATATCCAGAACCCTCTAAGAAGTTCCTGCTTTAATTTAGCTCCAAGA |
| | | ATGTGGTTAGAGACATGACCCTGCCGTGTACCAGCTGAGAGACTCTAAATCCAGTGACAAGTCTG |
| TC15P2_pool2_25 | 754 | GAGATCATGTCCTAACCCTGATCCTCTTGTCCCACAGATATCCAGAACCCTCTAAGAAGTTCCTGCTAATTTAAAGCTCCCTGA |
| | | ATGTGGTTAGAGACATGACCCTGCCGTGTACCAGCTGAGAGACTCTAAATCCAGTGACAAGTCTG |
| TC15P2_pool2_26 | 755 | GAGATCATGTCCTAACCCTGATCCTCTTGTCCCACAGATATCCAGAACCCTCTAAGAAGTTCCTGCTTATTTATAGCTCCGTGA |
| | | ATGTGGTTAGAGACATGACCCTGCCGTGTACCAGCTGAGAGACTCTAAATCCAGTGACAAGTCTG |
| TC15P2_pool2_27 | 756 | GAGATCATGTCCTAACCCTGATCCTCTTGTCCCACAGATATCCAGAACCCTCTAAGAAGTTCCTGCTTTTAATTAGCTCCTTGAA |
| | | TGTGGTTAGAGACATGACCCTGCCGTGTACCAGCTGAGAGACTCTAAATCCAGTGACAAGTCTG |
| TC15P2_pool2_28 | 757 | GAGATCATGTCCTAACCCTGATCCTCTTGTCCCACAGATATCCAGAACCCTCTAAGAAGTTCCTGCTTTAATATAGCTCGATGA |
| | | ATGTGGTTAGAGACATGACCCTGCCGTGTACCAGCTGAGAGACTCTAAATCCAGTGACAAGTCTG |
| TC15P2_pool2_29 | 758 | GAGATCATGTCCTAACCCTGATCCTCTTGTCCCACAGATATCCAGAACCCTCTAAGAAGTTCCTGCTAAATTTAAGCTCGCAGA |
| | | ATGTGGTTAGAGACATGACCCTGCCGTGTACCAGCTGAGAGACTCTAAATCCAGTGACAAGTCTG |
| TC15P2_pool2_30 | 759 | GAGATCATGTCCTAACCCTGATCCTCTTGTCCCACAGATATCCAGAACCCTCTAAGAAGTTCCTGCTATAAAAAAGCTCGGTGA |
| | | ATGTGGTTAGAGACATGACCCTGCCGTGTACCAGCTGAGAGACTCTAAATCCAGTGACAAGTCTG |
| TC15P2_pool2_31 | 760 | GAGATCATGTCCTAACCCTGATCCTCTTGTCCCACAGATATCCAGAACCCTCTAAGAAGTTCCTGCTTTAATAATGCTCGTAGA |
| | | ATGTGGTTAGAGACATGACCCTGCCGTGTACCAGCTGAGAGACTCTAAATCCAGTGACAAGTCTG |
| TC15P2_pool2_32 | 761 | GAGATCATGTCCTAACCCTGATCCTCTTGTCCCACAGATATCCAGAACCCTCTAAGAAGTTCCTGCTATTAATAAGCTCTATGA |
| | | ATGTGGTTAGAGACATGACCCTGCCGTGTACCAGCTGAGAGACTCTAAATCCAGTGACAAGTCTG |
| TC15P2_pool2_33 | 762 | GAGATCATGTCCTAACCCTGATCCTCTTGTCCCACAGATATCCAGAACCCTCTAAGAAGTTCCTGCTATATTAATGCTCTCAGA |
| | | ATGTGGTTAGAGACATGACCCTGCCGTGTACCAGCTGAGAGACTCTAAATCCAGTGACAAGTCTG |
| TC15P2_pool2_34 | 763 | GAGATCATGTCCTAACCCTGATCCTCTTGTCCCACAGATATCCAGAACCCTCTAAGAAGTTCCTGCTTTATAATTGCTCTGTGA |
| | | ATGTGGTTAGAGACATGACCCTGCCGTGTACCAGCTGAGAGACTCTAAATCCAGTGACAAGTCTG |
| TC15P2_pool2_35 | 764 | GAGATCATGTCCTAACCCTGATCCTCTTGTCCCACAGATATCCAGAACCCTCTAAGAAGTTCCTGCTTATATATTGCTCTTTGAA |
| | | TGTGGTTAGAGACATGACCCTGCCGTGTACCAGCTGAGAGACTCTAAATCCAGTGACAAGTCTG |
| TC15P2_pool2_36 | 765 | GAGATCATGTCCTAACCCTGATCCTCTTGTCCCACAGATATCCAGAACCCTCTAAGAAGTTCCTGCTTATATTTTGGTCAAAGA |
| | | ATGTGGTTAGAGACATGACCCTGCCGTGTACCAGCTGAGAGACTCTAAATCCAGTGACAAGTCTG |
| TC15P2_pool2_37 | 766 | GAGATCATGTCCTAACCCTGATCCTCTTGTCCCACAGATATCCAGAACCCTCTAAGAAGTTCCTGCTTAATATTAGGTCACTGA |
| | | ATGTGGTTAGAGACATGACCCTGCCGTGTACCAGCTGAGAGACTCTAAATCCAGTGACAAGTCTG |
| TC15P2_pool2_38 | 767 | GAGATCATGTCCTAACCCTGATCCTCTTGTCCCACAGATATCCAGAACCCTCTAAGAAGTTCCTGCTTAAAATTTGGTCAGAGA |
| | | ATGTGGTTAGAGACATGACCCTGCCGTGTACCAGCTGAGAGACTCTAAATCCAGTGACAAGTCTG |
| TC15P2_pool2_39 | 768 | GAGATCATGTCCTAACCCTGATCCTCTTGTCCCACAGATATCCAGAACCCTCTAAGAAGTTCCTGCTATATAATAGGTCATTGA |
| | | ATGTGGTTAGAGACATGACCCTGCCGTGTACCAGCTGAGAGACTCTAAATCCAGTGACAAGTCTG |
| TC15P2_pool2_40 | 769 | GAGATCATGTCCTAACCCTGATCCTCTTGTCCCACAGATATCCAGAACCCTCTAAGAAGTTCCTGCTAATTAAAAGGTCCAAGA |
| | | ATGTGGTTAGAGACATGACCCTGCCGTGTACCAGCTGAGAGACTCTAAATCCAGTGACAAGTCTG |
| TC15P2_pool2_41 | 770 | GAGATCATGTCCTAACCCTGATCCTCTTGTCCCACAGATATCCAGAACCCTCTAAGAAGTTCCTGCTAATAAAATGGTCCCTGA |
| | | ATGTGGTTAGAGACATGACCCTGCCGTGTACCAGCTGAGAGACTCTAAATCCAGTGACAAGTCTG |
| TC15P2_pool2_42 | 771 | GAGATCATGTCCTAACCCTGATCCTCTTGTCCCACAGATATCCAGAACCCTCTAAGAAGTTCCTGCTAAAAATAAGGTCCGTGA |
| | | ATGTGGTTAGAGACATGACCCTGCCGTGTACCAGCTGAGAGACTCTAAATCCAGTGACAAGTCTG |
| TC15P2_pool2_43 | 772 | GAGATCATGTCCTAACCCTGATCCTCTTGTCCCACAGATATCCAGAACCCTCTAAGAAGTTCCTGCTATTTTTAAGGTCCTTGA |
| | | ATGTGGTTAGAGACATGACCCTGCCGTGTACCAGCTGAGAGACTCTAAATCCAGTGACAAGTCTG |
| TC15P2_pool2_44 | 773 | GAGATCATGTCCTAACCCTGATCCTCTTGTCCCACAGATATCCAGAACCCTCTAAGAAGTTCCTGCTTTTTTAAAGGTCGATGA |
| | | ATGTGGTTAGAGACATGACCCTGCCGTGTACCAGCTGAGAGACTCTAAATCCAGTGACAAGTCTG |
| TC15P2_pool2_45 | 774 | GAGATCATGTCCTAACCCTGATCCTCTTGTCCCACAGATATCCAGAACCCTCTAAGAAGTTCCTGCTATAATTATGGTCGCAGA |
| | | ATGTGGTTAGAGACATGACCCTGCCGTGTACCAGCTGAGAGACTCTAAATCCAGTGACAAGTCTG |
| TC15P2_pool2_46 | 775 | GAGATCATGTCCTAACCCTGATCCTCTTGTCCCACAGATATCCAGAACCCTCTAAGAAGTTCCTGCTAATAAATAGGTCGGAGA |
| | | ATGTGGTTAGAGACATGACCCTGCCGTGTACCAGCTGAGAGACTCTAAATCCAGTGACAAGTCTG |
| TC15P2_pool2_47 | 776 | GAGATCATGTCCTAACCCTGATCCTCTTGTCCCACAGATATCCAGAACCCTCTAAGAAGTTCCTGCTTATATAATGGTCGTAGA |
| | | ATGTGGTTAGAGACATGACCCTGCCGTGTACCAGCTGAGAGACTCTAAATCCAGTGACAAGTCTG |
| TC15P2_pool2_48 | 777 | GAGATCATGTCCTAACCCTGATCCTCTTGTCCCACAGATATCCAGAACCCTCTAAGAAGTTCCTGCTTAAAAAAAGGTCTATGA |
| | | ATGTGGTTAGAGACATGACCCTGCCGTGTACCAGCTGAGAGACTCTAAATCCAGTGACAAGTCTG |
| TC15P2_pool2_49 | 778 | GAGATCATGTCCTAACCCTGATCCTCTTGTCCCACAGATATCCAGAACCCTCTAAGAAGTTCCTGCTATTTAATTGGTCTCAGA |
| | | ATGTGGTTAGAGACATGACCCTGCCGTGTACCAGCTGAGAGACTCTAAATCCAGTGACAAGTCTG |
| TC15P2_pool2_50 | 779 | GAGATCATGTCCTAACCCTGATCCTCTTGTCCCACAGATATCCAGAACCCTCTAAGAAGTTCCTGCTTTATAATAGGTCTGAGA |
| | | ATGTGGTTAGAGACATGACCCTGCCGTGTACCAGCTGAGAGACTCTAAATCCAGTGACAAGTCTG |
| TC15P2_pool2_51 | 780 | GAGATCATGTCCTAACCCTGATCCTCTTGTCCCACAGATATCCAGAACCCTCTAAGAAGTTCCTGCTTATATATAGGTCTTAGA |
| | | ATGTGGTTAGAGACATGACCCTGCCGTGTACCAGCTGAGAGACTCTAAATCCAGTGACAAGTCTG |
| TC15P2_pool2_52 | 781 | GAGATCATGTCCTAACCCTGATCCTCTTGTCCCACAGATATCCAGAACCCTCTAAGAAGTTCCTGCTAAAAAAAAGTTCAAAGA |
| | | ATGTGGTTAGAGACATGACCCTGCCGTGTACCAGCTGAGAGACTCTAAATCCAGTGACAAGTCTG |
| TC15P2_pool2_53 | 782 | GAGATCATGTCCTAACCCTGATCCTCTTGTCCCACAGATATCCAGAACCCTCTAAGAAGTTCCTGCTTATTTTAAGTTCACAGAA |
| | | TGTGGTTAGAGACATGACCCTGCCGTGTACCAGCTGAGAGACTCTAAATCCAGTGACAAGTCTG |
| TC15P2_pool2_54 | 783 | GAGATCATGTCCTAACCCTGATCCTCTTGTCCCACAGATATCCAGAACCCTCTAAGAAGTTCCTGCTAATTTTTAGTTCAGAGA |
| | | ATGTGGTTAGAGACATGACCCTGCCGTGTACCAGCTGAGAGACTCTAAATCCAGTGACAAGTCTG |
| TC15P2_pool2_55 | 784 | GAGATCATGTCCTAACCCTGATCCTCTTGTCCCACAGATATCCAGAACCCTCTAAGAAGTTCCTGCTAAATTATAGTTCATAGAA |
| | | TGTGGTTAGAGACATGACCCTGCCGTGTACCAGCTGAGAGACTCTAAATCCAGTGACAAGTCTG |
| TC15P2_pool2_56 | 785 | GAGATCATGTCCTAACCCTGATCCTCTTGTCCCACAGATATCCAGAACCCTCTAAGAAGTTCCTGCTTTTAAATTGTTCCATGAA |
| | | TGTGGTTAGAGACATGACCCTGCCGTGTACCAGCTGAGAGACTCTAAATCCAGTGACAAGTCTG |
| TC15P2_pool2_57 | 786 | GAGATCATGTCCTAACCCTGATCCTCTTGTCCCACAGATATCCAGAACCCTCTAAGAAGTTCCTGCTTTTAATTTGTTCCCAGAA |
| | | TGTGGTTAGAGACATGACCCTGCCGTGTACCAGCTGAGAGACTCTAAATCCAGTGACAAGTCTG |
| TC15P2_pool2_58 | 787 | GAGATCATGTCCTAACCCTGATCCTCTTGTCCCACAGATATCCAGAACCCTCTAAGAAGTTCCTGCTATAATTAAGTTCCGTGA |
| | | ATGTGGTTAGAGACATGACCCTGCCGTGTACCAGCTGAGAGACTCTAAATCCAGTGACAAGTCTG |
| TC15P2_pool2_59 | 788 | GAGATCATGTCCTAACCCTGATCCTCTTGTCCCACAGATATCCAGAACCCTCTAAGAAGTTCCTGCTTTAAAATTGTTCCTAGAA |
| | | TGTGGTTAGAGACATGACCCTGCCGTGTACCAGCTGAGAGACTCTAAATCCAGTGACAAGTCTG |
| TC15P2_pool2_60 | 789 | GAGATCATGTCCTAACCCTGATCCTCTTGTCCCACAGATATCCAGAACCCTCTAAGAAGTTCCTGCTTTATTTTTGTTCGATGAA |
| | | TGTGGTTAGAGACATGACCCTGCCGTGTACCAGCTGAGAGACTCTAAATCCAGTGACAAGTCTG |
| TC15P2_pool2_61 | 790 | GAGATCATGTCCTAACCCTGATCCTCTTGTCCCACAGATATCCAGAACCCTCTAAGAAGTTCCTGCTTAATTAAAGTTCGCTGA |
| | | ATGTGGTTAGAGACATGACCCTGCCGTGTACCAGCTGAGAGACTCTAAATCCAGTGACAAGTCTG |
| TC15P2_pool2_62 | 791 | GAGATCATGTCCTAACCCTGATCCTCTTGTCCCACAGATATCCAGAACCCTCTAAGAAGTTCCTGCTTATAAAAAGTTCGGAGA |
| | | ATGTGGTTAGAGACATGACCCTGCCGTGTACCAGCTGAGAGACTCTAAATCCAGTGACAAGTCTG |
| TC15P2_pool2_63 | 792 | GAGATCATGTCCTAACCCTGATCCTCTTGTCCCACAGATATCCAGAACCCTCTAAGAAGTTCCTGCTTATATTTAGTTCGTTGAA |
| | | TGTGGTTAGAGACATGACCCTGCCGTGTACCAGCTGAGAGACTCTAAATCCAGTGACAAGTCTG |
| TC15P2_pool2_64 | 793 | GAGATCATGTCCTAACCCTGATCCTCTTGTCCCACAGATATCCAGAACCCTCTAAGAAGTTCCTGCTTAAATAATGTTCTATGAA |
| | | TGTGGTTAGAGACATGACCCTGCCGTGTACCAGCTGAGAGACTCTAAATCCAGTGACAAGTCTG |
| TC15P2_pool2_65 | 794 | GAGATCATGTCCTAACCCTGATCCTCTTGTCCCACAGATATCCAGAACCCTCTAAGAAGTTCCTGCTAATTTTATGTTCTCAGAA |
| | | TGTGGTTAGAGACATGACCCTGCCGTGTACCAGCTGAGAGACTCTAAATCCAGTGACAAGTCTG |
| TC15P2_pool2_66 | 795 | GAGATCATGTCCTAACCCTGATCCTCTTGTCCCACAGATATCCAGAACCCTCTAAGAAGTTCCTGCTATTTATTAGTTCTGTGAA |
| | | TGTGGTTAGAGACATGACCCTGCCGTGTACCAGCTGAGAGACTCTAAATCCAGTGACAAGTCTG |
| TC15P2_pool2_67 | 796 | GAGATCATGTCCTAACCCTGATCCTCTTGTCCCACAGATATCCAGAACCCTCTAAGAAGTTCCTGCTAATTTTTTGTTCTTTGAA |
| | | TGTGGTTAGAGACATGACCCTGCCGTGTACCAGCTGAGAGACTCTAAATCCAGTGACAAGTCTG |
| TC15P3_pool2_68 | 797 | GAGATCATGTCCTAACCCTGATCCTCTTGTCCCACAGATATCCAGAACCCTCTAAGAAGTTCCTGCTTTTTAATATATCAATGAA |
| | | TGTGGTTAGAGACATGACCCTGCCGTGTACCAGCTGAGAGACTCTAAATCCAGTGACAAGTCTG |
| TC15P3_pool2_69 | 798 | GAGATCATGTCCTAACCCTGATCCTCTTGTCCCACAGATATCCAGAACCCTCTAAGAAGTTCCTGCTAAATTTTATATCACTGAA |
| | | TGTGGTTAGAGACATGACCCTGCCGTGTACCAGCTGAGAGACTCTAAATCCAGTGACAAGTCTG |
| TC15P3_pool2_70 | 799 | GAGATCATGTCCTAACCCTGATCCTCTTGTCCCACAGATATCCAGAACCCTCTAAGAAGTTCCTGCTTTTTAAATTATCAGTGAA |
| | | TGTGGTTAGAGACATGACCCTGCCGTGTACCAGCTGAGAGACTCTAAATCCAGTGACAAGTCTG |
| TC15P3_pool2_71 | 800 | GAGATCATGTCCTAACCCTGATCCTCTTGTCCCACAGATATCCAGAACCCTCTAAGAAGTTCCTGCTATATTTTTTATCATAGAA |
| | | TGTGGTTAGAGACATGACCCTGCCGTGTACCAGCTGAGAGACTCTAAATCCAGTGACAAGTCTG |
| TC15P3_pool2_72 | 801 | GAGATCATGTCCTAACCCTGATCCTCTTGTCCCACAGATATCCAGAACCCTCTAAGAAGTTCCTGCTTATAAATTTATCCAAGAA |
| | | TGTGGTTAGAGACATGACCCTGCCGTGTACCAGCTGAGAGACTCTAAATCCAGTGACAAGTCTG |
| TC15P3_pool2_73 | 802 | GAGATCATGTCCTAACCCTGATCCTCTTGTCCCACAGATATCCAGAACCCTCTAAGAAGTTCCTGCTAATATTAATATCCCTGAA |
| | | TGTGGTTAGAGACATGACCCTGCCGTGTACCAGCTGAGAGACTCTAAATCCAGTGACAAGTCTG |
| TC15P3_pool2_74 | 803 | GAGATCATGTCCTAACCCTGATCCTCTTGTCCCACAGATATCCAGAACCCTCTAAGAAGTTCCTGCTAAATTTATTATCCGTGAA |
| | | TGTGGTTAGAGACATGACCCTGCCGTGTACCAGCTGAGAGACTCTAAATCCAGTGACAAGTCTG |
| TC15P3_pool2_75 | 804 | GAGATCATGTCCTAACCCTGATCCTCTTGTCCCACAGATATCCAGAACCCTCTAAGAAGTTCCTGCTTTATAAAATATCCTTGAA |
| | | TGTGGTTAGAGACATGACCCTGCCGTGTACCAGCTGAGAGACTCTAAATCCAGTGACAAGTCTG |
| TC15P3_pool2_76 | 805 | GAGATCATGTCCTAACCCTGATCCTCTTGTCCCACAGATATCCAGAACCCTCTAAGAAGTTCCTGCTTAAAAATATATCGAAGA |
| | | ATGTGGTTAGAGACATGACCCTGCCGTGTACCAGCTGAGAGACTCTAAATCCAGTGACAAGTCTG |
| TC15P3_pool2_77 | 806 | GAGATCATGTCCTAACCCTGATCCTCTTGTCCCACAGATATCCAGAACCCTCTAAGAAGTTCCTGCTATTAATTTTATCGCTGAA |
| | | TGTGGTTAGAGACATGACCCTGCCGTGTACCAGCTGAGAGACTCTAAATCCAGTGACAAGTCTG |
| TC15P3_pool2_78 | 807 | GAGATCATGTCCTAACCCTGATCCTCTTGTCCCACAGATATCCAGAACCCTCTAAGAAGTTCCTGCTTATATAAATATCGGTGA |
| | | ATGTGGTTAGAGACATGACCCTGCCGTGTACCAGCTGAGAGACTCTAAATCCAGTGACAAGTCTG |
| TC15P3_pool2_79 | 808 | GAGATCATGTCCTAACCCTGATCCTCTTGTCCCACAGATATCCAGAACCCTCTAAGAAGTTCCTGCTATAAATTATATCGTTGAA |
| | | TGTGGTTAGAGACATGACCCTGCCGTGTACCAGCTGAGAGACTCTAAATCCAGTGACAAGTCTG |
| TC15P3_pool2_80 | 809 | GAGATCATGTCCTAACCCTGATCCTCTTGTCCCACAGATATCCAGAACCCTCTAAGAAGTTCCTGCTTTTATTATTATCTATGAA |
| | | TGTGGTTAGAGACATGACCCTGCCGTGTACCAGCTGAGAGACTCTAAATCCAGTGACAAGTCTG |
| TC15P3_pool2_81 | 810 | GAGATCATGTCCTAACCCTGATCCTCTTGTCCCACAGATATCCAGAACCCTCTAAGAAGTTCCTGCTATAAATTTTATCTCAGAA |
| | | TGTGGTTAGAGACATGACCCTGCCGTGTACCAGCTGAGAGACTCTAAATCCAGTGACAAGTCTG |
| TC15P3_pool2_82 | 811 | GAGATCATGTCCTAACCCTGATCCTCTTGTCCCACAGATATCCAGAACCCTCTAAGAAGTTCCTGCTATTATTTATATCTGTGAA |
| | | TGTGGTTAGAGACATGACCCTGCCGTGTACCAGCTGAGAGACTCTAAATCCAGTGACAAGTCTG |
| TC15P3_pool2_83 | 812 | GAGATCATGTCCTAACCCTGATCCTCTTGTCCCACAGATATCCAGAACCCTCTAAGAAGTTCCTGCTAAATAAAATATCTTTGAA |
| | | TGTGGTTAGAGACATGACCCTGCCGTGTACCAGCTGAGAGACTCTAAATCCAGTGACAAGTCTG |
| TC15P3_pool2_84 | 813 | GAGATCATGTCCTAACCCTGATCCTCTTGTCCCACAGATATCCAGAACCCTCTAAGAAGTTCCTGCTTAATTAATTCTCAAAGAA |
| | | TGTGGTTAGAGACATGACCCTGCCGTGTACCAGCTGAGAGACTCTAAATCCAGTGACAAGTCTG |
| TC15P3_pool2_85 | 814 | GAGATCATGTCCTAACCCTGATCCTCTTGTCCCACAGATATCCAGAACCCTCTAAGAAGTTCCTGCTAATTATAATCTCACTGAA |
| | | TGTGGTTAGAGACATGACCCTGCCGTGTACCAGCTGAGAGACTCTAAATCCAGTGACAAGTCTG |
| TC15P3_pool2_86 | 815 | GAGATCATGTCCTAACCCTGATCCTCTTGTCCCACAGATATCCAGAACCCTCTAAGAAGTTCCTGCTATATTATTTCTCAGTGAA |
| | | TGTGGTTAGAGACATGACCCTGCCGTGTACCAGCTGAGAGACTCTAAATCCAGTGACAAGTCTG |
| TC15P3_pool2_87 | 816 | GAGATCATGTCCTAACCCTGATCCTCTTGTCCCACAGATATCCAGAACCCTCTAAGAAGTTCCTGCTTTATAAATTCTCATAGAA |
| | | TGTGGTTAGAGACATGACCCTGCCGTGTACCAGCTGAGAGACTCTAAATCCAGTGACAAGTCTG |
| TC15P3_pool2_88 | 817 | GAGATCATGTCCTAACCCTGATCCTCTTGTCCCACAGATATCCAGAACCCTCTAAGAAGTTCCTGCTTTTTATATTCTCCAAGAA |
| | | TGTGGTTAGAGACATGACCCTGCCGTGTACCAGCTGAGAGACTCTAAATCCAGTGACAAGTCTG |
| TC15P3_pool2_89 | 818 | GAGATCATGTCCTAACCCTGATCCTCTTGTCCCACAGATATCCAGAACCCTCTAAGAAGTTCCTGCTAAAATTTTTCTCCCAGA |
| | | ATGTGGTTAGAGACATGACCCTGCCGTGTACCAGCTGAGAGACTCTAAATCCAGTGACAAGTCTG |
| TC15P3_pool2_90 | 819 | GAGATCATGTCCTAACCCTGATCCTCTTGTCCCACAGATATCCAGAACCCTCTAAGAAGTTCCTGCTTATTAAAATCTCCGTGA |
| | | ATGTGGTTAGAGACATGACCCTGCCGTGTACCAGCTGAGAGACTCTAAATCCAGTGACAAGTCTG |
| TC15P3_pool2_91 | 820 | GAGATCATGTCCTAACCCTGATCCTCTTGTCCCACAGATATCCAGAACCCTCTAAGAAGTTCCTGCTTAAATTAATCTCCTAGA/ |
| | | TGTGGTTAGAGACATGACCCTGCCGTGTACCAGCTGAGAGACTCTAAATCCAGTGACAAGTCTG |
| TC15P3_pool2_92 | 821 | GAGATCATGTCCTAACCCTGATCCTCTTGTCCCACAGATATCCAGAACCCTCTAAGAAGTTCCTGCTTAAATATTTCTCGAAGA |
| | | ATGTGGTTAGAGACATGACCCTGCCGTGTACCAGCTGAGAGACTCTAAATCCAGTGACAAGTCTG |
| TC15P3_pool2_93 | 822 | GAGATCATGTCCTAACCCTGATCCTCTTGTCCCACAGATATCCAGAACCCTCTAAGAAGTTCCTGCTAATAATTATCTCGCTGA |
| | | ATGTGGTTAGAGACATGACCCTGCCGTGTACCAGCTGAGAGACTCTAAATCCAGTGACAAGTCTG |
| TC15P3_pool2_94 | 823 | GAGATCATGTCCTAACCCTGATCCTCTTGTCCCACAGATATCCAGAACCCTCTAAGAAGTTCCTGCTATATAAATTCTCGGTGA |
| | | ATGTGGTTAGAGACATGACCCTGCCGTGTACCAGCTGAGAGACTCTAAATCCAGTGACAAGTCTG |
| TC15P3_pool2_95 | 824 | GAGATCATGTCCTAACCCTGATCCTCTTGTCCCACAGATATCCAGAACCCTCTAAGAAGTTCCTGCTTTAAATATTCTCGTTGAA |
| | | TGTGGTTAGAGACATGACCCTGCCGTGTACCAGCTGAGAGACTCTAAATCCAGTGACAAGTCTG |
| TC15P3_pool2_96 | 825 | GAGATCATGTCCTAACCCTGATCCTCTTGTCCCACAGATATCCAGAACCCTCTAAGAAGTTCCTGCTAAAAATATTCTCTAAGAA |
| | | TGTGGTTAGAGACATGACCCTGCCGTGTACCAGCTGAGAGACTCTAAATCCAGTGACAAGTCTG |
| TC15P3_pool2_97 | 826 | GAGATCATGTCCTAACCCTGATCCTCTTGTCCCACAGATATCCAGAACCCTCTAAGAAGTTCCTGCTATATATATTCTCTCAGAA |
| | | TGTGGTTAGAGACATGACCCTGCCGTGTACCAGCTGAGAGACTCTAAATCCAGTGACAAGTCTG |
| TC15P3_pool2_98 | 827 | GAGATCATGTCCTAACCCTGATCCTCTTGTCCCACAGATATCCAGAACCCTCTAAGAAGTTCCTGCTTTATTAATTCTCTGTGAA |
| | | TGTGGTTAGAGACATGACCCTGCCGTGTACCAGCTGAGAGACTCTAAATCCAGTGACAAGTCTG |
| TC15P3_pool2_99 | 828 | GAGATCATGTCCTAACCCTGATCCTCTTGTCCCACAGATATCCAGAACCCTCTAAGAAGTTCCTGCTATTATAAATCTCTTAGAA |
| | | TGTGGTTAGAGACATGACCCTGCCGTGTACCAGCTGAGAGACTCTAAATCCAGTGACAAGTCTG |
| TC15P3_pool2_100 | 829 | GAGATCATGTCCTAACCCTGATCCTCTTGTCCCACAGATATCCAGAACCCTCTAAGAAGTTCCTGCTTTTAAAAATGTCAATGAA |
| | | TGTGGTTAGAGACATGACCCTGCCGTGTACCAGCTGAGAGACTCTAAATCCAGTGACAAGTCTG |
| TC15P3_pool2_101 | 830 | GAGATCATGTCCTAACCCTGATCCTCTTGTCCCACAGATATCCAGAACCCTCTAAGAAGTTCCTGCTTATAAATATGTCACTGA |
| | | ATGTGGTTAGAGACATGACCCTGCCGTGTACCAGCTGAGAGACTCTAAATCCAGTGACAAGTCTG |
| TC15P3_pool2_102 | 831 | GAGATCATGTCCTAACCCTGATCCTCTTGTCCCACAGATATCCAGAACCCTCTAAGAAGTTCCTGCTTTTAATAATGTCAGAGA |
| | | ATGTGGTTAGAGACATGACCCTGCCGTGTACCAGCTGAGAGACTCTAAATCCAGTGACAAGTCTG |
| TC15P3_pool2_103 | 832 | GAGATCATGTCCTAACCCTGATCCTCTTGTCCCACAGATATCCAGAACCCTCTAAGAAGTTCCTGCTATAATTTTTGTCATTGAA |
| | | TGTGGTTAGAGACATGACCCTGCCGTGTACCAGCTGAGAGACTCTAAATCCAGTGACAAGTCTG |
| TC15P3_pool2_104 | 833 | GAGATCATGTCCTAACCCTGATCCTCTTGTCCCACAGATATCCAGAACCCTCTAAGAAGTTCCTGCTTTTTTATTTGTCCAAGAA |
| | | TGTGGTTAGAGACATGACCCTGCCGTGTACCAGCTGAGAGACTCTAAATCCAGTGACAAGTCTG |
| TC15P3_pool2_105 | 834 | GAGATCATGTCCTAACCCTGATCCTCTTGTCCCACAGATATCCAGAACCCTCTAAGAAGTTCCTGCTTAATTATTTGTCCCTGAA |
| | | TGTGGTTAGAGACATGACCCTGCCGTGTACCAGCTGAGAGACTCTAAATCCAGTGACAAGTCTG |
| TC15P3_pool2_106 | 835 | GAGATCATGTCCTAACCCTGATCCTCTTGTCCCACAGATATCCAGAACCCTCTAAGAAGTTCCTGCTATAAAAATTGTCCGAGA |
| | | ATGTGGTTAGAGACATGACCCTGCCGTGTACCAGCTGAGAGACTCTAAATCCAGTGACAAGTCTG |
| TC15P3_pool2_107 | 836 | GAGATCATGTCCTAACCCTGATCCTCTTGTCCCACAGATATCCAGAACCCTCTAAGAAGTTCCTGCTTATAATATTGTCCTAGAA |
| | | TGTGGTTAGAGACATGACCCTGCCGTGTACCAGCTGAGAGACTCTAAATCCAGTGACAAGTCTG |
| TC15P3_pool2_108 | 837 | GAGATCATGTCCTAACCCTGATCCTCTTGTCCCACAGATATCCAGAACCCTCTAAGAAGTTCCTGCTTAATATATTGTCGAAGA |
| | | ATGTGGTTAGAGACATGACCCTGCCGTGTACCAGCTGAGAGACTCTAAATCCAGTGACAAGTCTG |
| TC15P3_pool2_109 | 838 | GAGATCATGTCCTAACCCTGATCCTCTTGTCCCACAGATATCCAGAACCCTCTAAGAAGTTCCTGCTTAATTTTTTGTCGCAGA |
| | | ATGTGGTTAGAGACATGACCCTGCCGTGTACCAGCTGAGAGACTCTAAATCCAGTGACAAGTCTG |
| TC15P3_pool2_110 | 839 | GAGATCATGTCCTAACCCTGATCCTCTTGTCCCACAGATATCCAGAACCCTCTAAGAAGTTCCTGCTATTTAAAATGTCGGTGA |
| | | ATGTGGTTAGAGACATGACCCTGCCGTGTACCAGCTGAGAGACTCTAAATCCAGTGACAAGTCTG |
| TC15P3_pool2_111 | 840 | GAGATCATGTCCTAACCCTGATCCTCTTGTCCCACAGATATCCAGAACCCTCTAAGAAGTTCCTGCTATATAAAATGTCGTAGA |
| | | ATGTGGTTAGAGACATGACCCTGCCGTGTACCAGCTGAGAGACTCTAAATCCAGTGACAAGTCTG |
| TC15P3_pool2_112 | 841 | GAGATCATGTCCTAACCCTGATCCTCTTGTCCCACAGATATCCAGAACCCTCTAAGAAGTTCCTGCTATAATATTTGTCTAAGAA |
| | | TGTGGTTAGAGACATGACCCTGCCGTGTACCAGCTGAGAGACTCTAAATCCAGTGACAAGTCTG |
| TC15P3_pool2_113 | 842 | GAGATCATGTCCTAACCCTGATCCTCTTGTCCCACAGATATCCAGAACCCTCTAAGAAGTTCCTGCTATTATTTTTGTCTCAGAA |
| | | TGTGGTTAGAGACATGACCCTGCCGTGTACCAGCTGAGAGACTCTAAATCCAGTGACAAGTCTG |
| TC15P3_pool2_114 | 843 | GAGATCATGTCCTAACCCTGATCCTCTTGTCCCACAGATATCCAGAACCCTCTAAGAAGTTCCTGCTAATTAATATGTCTGTGA |
| | | ATGTGGTTAGAGACATGACCCTGCCGTGTACCAGCTGAGAGACTCTAAATCCAGTGACAAGTCTG |
| TC15P3_pool2_115 | 844 | GAGATCATGTCCTAACCCTGATCCTCTTGTCCCACAGATATCCAGAACCCTCTAAGAAGTTCCTGCTTATTATTATGTCTTAGAA |
| | | TGTGGTTAGAGACATGACCCTGCCGTGTACCAGCTGAGAGACTCTAAATCCAGTGACAAGTCTG |
| TC15P3_pool2_116 | 845 | GAGATCATGTCCTAACCCTGATCCTCTTGTCCCACAGATATCCAGAACCCTCTAAGAAGTTCCTGCTATTAAATATTTCAATGAA |
| | | TGTGGTTAGAGACATGACCCTGCCGTGTACCAGCTGAGAGACTCTAAATCCAGTGACAAGTCTG |
| TC15P3_pool2_117 | 846 | GAGATCATGTCCTAACCCTGATCCTCTTGTCCCACAGATATCCAGAACCCTCTAAGAAGTTCCTGCTAAAAAATTTTTCACTGAA |
| | | TGTGGTTAGAGACATGACCCTGCCGTGTACCAGCTGAGAGACTCTAAATCCAGTGACAAGTCTG |
| TC15P3_pool2_118 | 847 | GAGATCATGTCCTAACCCTGATCCTCTTGTCCCACAGATATCCAGAACCCTCTAAGAAGTTCCTGCTTTTTAAAATTTCAGAGAA |
| | | TGTGGTTAGAGACATGACCCTGCCGTGTACCAGCTGAGAGACTCTAAATCCAGTGACAAGTCTG |
| TC15P3_pool2_119 | 848 | GAGATCATGTCCTAACCCTGATCCTCTTGTCCCACAGATATCCAGAACCCTCTAAGAAGTTCCTGCTTAAATTTTTTTCATTGAA |
| | | TGTGGTTAGAGACATGACCCTGCCGTGTACCAGCTGAGAGACTCTAAATCCAGTGACAAGTCTG |
| TC15P3_pool2_120 | 849 | GAGATCATGTCCTAACCCTGATCCTCTTGTCCCACAGATATCCAGAACCCTCTAAGAAGTTCCTGCTTTATTTATTTTCCAAGAA |
| | | TGTGGTTAGAGACATGACCCTGCCGTGTACCAGCTGAGAGACTCTAAATCCAGTGACAAGTCTG |
| TC15P3_pool2_121 | 850 | GAGATCATGTCCTAACCCTGATCCTCTTGTCCCACAGATATCCAGAACCCTCTAAGAAGTTCCTGCTATAAAATATTTCCCAGA |
| | | ATGTGGTTAGAGACATGACCCTGCCGTGTACCAGCTGAGAGACTCTAAATCCAGTGACAAGTCTG |
| TC15P3_pool2_122 | 851 | GAGATCATGTCCTAACCCTGATCCTCTTGTCCCACAGATATCCAGAACCCTCTAAGAAGTTCCTGCTTTTTTTTATTTCCGAGAA |
| | | TGTGGTTAGAGACATGACCCTGCCGTGTACCAGCTGAGAGACTCTAAATCCAGTGACAAGTCTG |
| TC15P3_pool2_123 | 852 | GAGATCATGTCCTAACCCTGATCCTCTTGTCCCACAGATATCCAGAACCCTCTAAGAAGTTCCTGCTTATTAAATTTTCCTAGAA |
| | | TGTGGTTAGAGACATGACCCTGCCGTGTACCAGCTGAGAGACTCTAAATCCAGTGACAAGTCTG |
| TC15P3_pool2_124 | 853 | GAGATCATGTCCTAACCCTGATCCTCTTGTCCCACAGATATCCAGAACCCTCTAAGAAGTTCCTGCTATTAATTATTTCGAAGAA |
| | | TGTGGTTAGAGACATGACCCTGCCGTGTACCAGCTGAGAGACTCTAAATCCAGTGACAAGTCTG |
| TC15P3_pool2_125 | 854 | GAGATCATGTCCTAACCCTGATCCTCTTGTCCCACAGATATCCAGAACCCTCTAAGAAGTTCCTGCTAAATATTATTTCGCAGA |
| | | ATGTGGTTAGAGACATGACCCTGCCGTGTACCAGCTGAGAGACTCTAAATCCAGTGACAAGTCTG |
| TC15P3_pool3_1 | 855 | GAGATCATGTCCTAACCCTGATCCTCTTGTCCCACAGATATCCAGAACCCTCTAAGAAGTTCCTGCTTTTAAAATTTTCGGAGA |
| | | ATGTGGTTAGAGACATGACCCTGCCGTGTACCAGCTGAGAGACTCTAAATCCAGTGACAAGTCTG |
| TC15P3_pool3_2 | 856 | GAGATCATGTCCTAACCCTGATCCTCTTGTCCCACAGATATCCAGAACCCTCTAAGAAGTTCCTGCTATTTTAATTTTCGTTGAA |
| | | TGTGGTTAGAGACATGACCCTGCCGTGTACCAGCTGAGAGACTCTAAATCCAGTGACAAGTCTG |
| TC15P3_pool3_3 | 857 | GAGATCATGTCCTAACCCTGATCCTCTTGTCCCACAGATATCCAGAACCCTCTAAGAAGTTCCTGCTATAAATAATTTCTAAGAA |
| | | TGTGGTTAGAGACATGACCCTGCCGTGTACCAGCTGAGAGACTCTAAATCCAGTGACAAGTCTG |
| TC15P3_pool3_4 | 858 | GAGATCATGTCCTAACCCTGATCCTCTTGTCCCACAGATATCCAGAACCCTCTAAGAAGTTCCTGCTTTATATTTTTTCTCAGAA |
| | | TGTGGTTAGAGACATGACCCTGCCGTGTACCAGCTGAGAGACTCTAAATCCAGTGACAAGTCTG |
| TC15P3_pool3_5 | 859 | GAGATCATGTCCTAACCCTGATCCTCTTGTCCCACAGATATCCAGAACCCTCTAAGAAGTTCCTGCTAATATATTTTTCTGAGAA |
| | | TGTGGTTAGAGACATGACCCTGCCGTGTACCAGCTGAGAGACTCTAAATCCAGTGACAAGTCTG |
| TC15P3_pool3_6 | 860 | GAGATCATGTCCTAACCCTGATCCTCTTGTCCCACAGATATCCAGAACCCTCTAAGAAGTTCCTGCTTTTAAATATTTCTTAGAA |
| | | TGTGGTTAGAGACATGACCCTGCCGTGTACCAGCTGAGAGACTCTAAATCCAGTGACAAGTCTG |

TABLE 17
library comprising 256 target sequences (ssODN) with 13 bp spacers designed to test TC context in Example 6

| | SEQ | |
|---|---|---|
| Name | ID # | Sequence |
| TC13P1_pool1_1 | 861 | GAGATCATGTCCTAACCCTGATCCTCTTGTCCCACAGATATCCAGAACCCTCTAAGAAGTTCCTGCTTTAATAAATCAAAGAAT |
| | | GTGGTTAGAGACATGACCCTGCCGTGTACCAGCTGAGAGACTCTAAATCCAGTGACAAGTCTG |
| TC13P1_pool1_2 | 862 | GAGATCATGTCCTAACCCTGATCCTCTTGTCCCACAGATATCCAGAACCCTCTAAGAAGTTCCTGCTTAATTTAATCACTGAAT |
| | | GTGGTTAGAGACATGACCCTGCCGTGTACCAGCTGAGAGACTCTAAATCCAGTGACAAGTCTG |
| TC13P1_pool1_3 | 863 | GAGATCATGTCCTAACCCTGATCCTCTTGTCCCACAGATATCCAGAACCCTCTAAGAAGTTCCTGCTAATTTTAATCAGAGAAT |
| | | GTGGTTAGAGACATGACCCTGCCGTGTACCAGCTGAGAGACTCTAAATCCAGTGACAAGTCTG |
| TC13P1_pool1_4 | 864 | GAGATCATGTCCTAACCCTGATCCTCTTGTCCCACAGATATCCAGAACCCTCTAAGAAGTTCCTGCTTTATATAATCATAGAAT |
| | | GTGGTTAGAGACATGACCCTGCCGTGTACCAGCTGAGAGACTCTAAATCCAGTGACAAGTCTG |
| TC13P1_pool1_5 | 865 | GAGATCATGTCCTAACCCTGATCCTCTTGTCCCACAGATATCCAGAACCCTCTAAGAAGTTCCTGCTAATTATAATCCATGAAT |
| | | GTGGTTAGAGACATGACCCTGCCGTGTACCAGCTGAGAGACTCTAAATCCAGTGACAAGTCTG |
| TC13P1_pool1_6 | 866 | GAGATCATGTCCTAACCCTGATCCTCTTGTCCCACAGATATCCAGAACCCTCTAAGAAGTTCCTGCTTTTTAAAATCCCAGAAT |
| | | GTGGTTAGAGACATGACCCTGCCGTGTACCAGCTGAGAGACTCTAAATCCAGTGACAAGTCTG |
| TC13P1_pool1_7 | 867 | GAGATCATGTCCTAACCCTGATCCTCTTGTCCCACAGATATCCAGAACCCTCTAAGAAGTTCCTGCTATTTATAATCCGAGAAT |
| | | GTGGTTAGAGACATGACCCTGCCGTGTACCAGCTGAGAGACTCTAAATCCAGTGACAAGTCTG |
| TC13P1_pool1_8 | 868 | GAGATCATGTCCTAACCCTGATCCTCTTGTCCCACAGATATCCAGAACCCTCTAAGAAGTTCCTGCTAAATTAAATCCTTGAAT |
| | | GTGGTTAGAGACATGACCCTGCCGTGTACCAGCTGAGAGACTCTAAATCCAGTGACAAGTCTG |
| TC13P1_pool1_9 | 869 | GAGATCATGTCCTAACCCTGATCCTCTTGTCCCACAGATATCCAGAACCCTCTAAGAAGTTCCTGCTATAAAAAATCGAAGAA |
| | | TGTGGTTAGAGACATGACCCTGCCGTGTACCAGCTGAGAGACTCTAAATCCAGTGACAAGTCTG |
| TC13P1_pool1_10 | 870 | GAGATCATGTCCTAACCCTGATCCTCTTGTCCCACAGATATCCAGAACCCTCTAAGAAGTTCCTGCTTATAATAATCGCTGAAT |
| | | GTGGTTAGAGACATGACCCTGCCGTGTACCAGCTGAGAGACTCTAAATCCAGTGACAAGTCTG |
| TC13P1_pool1_11 | 871 | GAGATCATGTCCTAACCCTGATCCTCTTGTCCCACAGATATCCAGAACCCTCTAAGAAGTTCCTGCTAAAAATAATCGGTGAA |
| | | TGTGGTTAGAGACATGACCCTGCCGTGTACCAGCTGAGAGACTCTAAATCCAGTGACAAGTCTG |
| TC13P1_pool1_12 | 872 | GAGATCATGTCCTAACCCTGATCCTCTTGTCCCACAGATATCCAGAACCCTCTAAGAAGTTCCTGCTTTTATTAATCGTAGAAT |
| | | GTGGTTAGAGACATGACCCTGCCGTGTACCAGCTGAGAGACTCTAAATCCAGTGACAAGTCTG |
| TC13P1_pool1_13 | 873 | GAGATCATGTCCTAACCCTGATCCTCTTGTCCCACAGATATCCAGAACCCTCTAAGAAGTTCCTGCTAATAAAAATCTATGAAT |
| | | GTGGTTAGAGACATGACCCTGCCGTGTACCAGCTGAGAGACTCTAAATCCAGTGACAAGTCTG |
| TC13P1_pool1_14 | 874 | GAGATCATGTCCTAACCCTGATCCTCTTGTCCCACAGATATCCAGAACCCTCTAAGAAGTTCCTGCTTATTTAAATCTCAGAAT |
| | | GTGGTTAGAGACATGACCCTGCCGTGTACCAGCTGAGAGACTCTAAATCCAGTGACAAGTCTG |
| TC13P1_pool1_15 | 875 | GAGATCATGTCCTAACCCTGATCCTCTTGTCCCACAGATATCCAGAACCCTCTAAGAAGTTCCTGCTTATTAAAATCTGTGAAT |
| | | GTGGTTAGAGACATGACCCTGCCGTGTACCAGCTGAGAGACTCTAAATCCAGTGACAAGTCTG |
| TC13P1_pool1_16 | 876 | GAGATCATGTCCTAACCCTGATCCTCTTGTCCCACAGATATCCAGAACCCTCTAAGAAGTTCCTGCTTTTATAAATCTTTGAAT |
| | | GTGGTTAGAGACATGACCCTGCCGTGTACCAGCTGAGAGACTCTAAATCCAGTGACAAGTCTG |
| TC13P1_pool1_17 | 877 | GAGATCATGTCCTAACCCTGATCCTCTTGTCCCACAGATATCCAGAACCCTCTAAGAAGTTCCTGCTTATTATACTCAAAGAAT |
| | | GTGGTTAGAGACATGACCCTGCCGTGTACCAGCTGAGAGACTCTAAATCCAGTGACAAGTCTG |
| TC13P1_pool1_18 | 878 | GAGATCATGTCCTAACCCTGATCCTCTTGTCCCACAGATATCCAGAACCCTCTAAGAAGTTCCTGCTATATATACTCACTGAAT |
| | | GTGGTTAGAGACATGACCCTGCCGTGTACCAGCTGAGAGACTCTAAATCCAGTGACAAGTCTG |
| TC13P1_pool1_19 | 879 | GAGATCATGTCCTAACCCTGATCCTCTTGTCCCACAGATATCCAGAACCCTCTAAGAAGTTCCTGCTATATTTACTCAGAGAAT |
| | | GTGGTTAGAGACATGACCCTGCCGTGTACCAGCTGAGAGACTCTAAATCCAGTGACAAGTCTG |
| TC13P1_pool1_20 | 880 | GAGATCATGTCCTAACCCTGATCCTCTTGTCCCACAGATATCCAGAACCCTCTAAGAAGTTCCTGCTATTATAACTCATAGAAT |
| | | GTGGTTAGAGACATGACCCTGCCGTGTACCAGCTGAGAGACTCTAAATCCAGTGACAAGTCTG |
| TC13P1_pool1_21 | 881 | GAGATCATGTCCTAACCCTGATCCTCTTGTCCCACAGATATCCAGAACCCTCTAAGAAGTTCCTGCTAATAATACTCCAAGAA |
| | | TGTGGTTAGAGACATGACCCTGCCGTGTACCAGCTGAGAGACTCTAAATCCAGTGACAAGTCTG |
| TC13P1_pool1_22 | 882 | GAGATCATGTCCTAACCCTGATCCTCTTGTCCCACAGATATCCAGAACCCTCTAAGAAGTTCCTGCTATAATTACTCCCTGAAT |
| | | GTGGTTAGAGACATGACCCTGCCGTGTACCAGCTGAGAGACTCTAAATCCAGTGACAAGTCTG |
| TC13P1_pool1_23 | 883 | GAGATCATGTCCTAACCCTGATCCTCTTGTCCCACAGATATCCAGAACCCTCTAAGAAGTTCCTGCTAAATATACTCCGAGAA |
| | | TGTGGTTAGAGACATGACCCTGCCGTGTACCAGCTGAGAGACTCTAAATCCAGTGACAAGTCTG |
| TC13P1_pool1_24 | 884 | GAGATCATGTCCTAACCCTGATCCTCTTGTCCCACAGATATCCAGAACCCTCTAAGAAGTTCCTGCTAAAATAACTCCTAGAA |
| | | TGTGGTTAGAGACATGACCCTGCCGTGTACCAGCTGAGAGACTCTAAATCCAGTGACAAGTCTG |
| TC13P1_pool1_25 | 885 | GAGATCATGTCCTAACCCTGATCCTCTTGTCCCACAGATATCCAGAACCCTCTAAGAAGTTCCTGCTATTTTAACTCGATGAAT |
| | | GTGGTTAGAGACATGACCCTGCCGTGTACCAGCTGAGAGACTCTAAATCCAGTGACAAGTCTG |
| TC13P1_pool1_26 | 886 | GAGATCATGTCCTAACCCTGATCCTCTTGTCCCACAGATATCCAGAACCCTCTAAGAAGTTCCTGCTTTTTATACTCGCTGAAT |
| | | GTGGTTAGAGACATGACCCTGCCGTGTACCAGCTGAGAGACTCTAAATCCAGTGACAAGTCTG |
| TC13P1_pool1_27 | 887 | GAGATCATGTCCTAACCCTGATCCTCTTGTCCCACAGATATCCAGAACCCTCTAAGAAGTTCCTGCTTAAAATACTCGGAGAA |
| | | TGTGGTTAGAGACATGACCCTGCCGTGTACCAGCTGAGAGACTCTAAATCCAGTGACAAGTCTG |
| TC13P1_pool1_28 | 888 | GAGATCATGTCCTAACCCTGATCCTCTTGTCCCACAGATATCCAGAACCCTCTAAGAAGTTCCTGCTATTAATACTCGTTGAAT |
| | | GTGGTTAGAGACATGACCCTGCCGTGTACCAGCTGAGAGACTCTAAATCCAGTGACAAGTCTG |
| TC13P1_pool1_29 | 889 | GAGATCATGTCCTAACCCTGATCCTCTTGTCCCACAGATATCCAGAACCCTCTAAGAAGTTCCTGCTTAATAAACTCTAAGAAT |
| | | GTGGTTAGAGACATGACCCTGCCGTGTACCAGCTGAGAGACTCTAAATCCAGTGACAAGTCTG |
| TC13P1_pool1_30 | 890 | GAGATCATGTCCTAACCCTGATCCTCTTGTCCCACAGATATCCAGAACCCTCTAAGAAGTTCCTGCTTAAATAACTCTCTGAAT |
| | | GTGGTTAGAGACATGACCCTGCCGTGTACCAGCTGAGAGACTCTAAATCCAGTGACAAGTCTG |
| TC13P1_pool1_31 | 891 | GAGATCATGTCCTAACCCTGATCCTCTTGTCCCACAGATATCCAGAACCCTCTAAGAAGTTCCTGCTTATAAAACTCTGAGAA |
| | | TGTGGTTAGAGACATGACCCTGCCGTGTACCAGCTGAGAGACTCTAAATCCAGTGACAAGTCTG |
| TC13P1_pool1_32 | 892 | GAGATCATGTCCTAACCCTGATCCTCTTGTCCCACAGATATCCAGAACCCTCTAAGAAGTTCCTGCTTTAAATACTCTTTGAAT |
| | | GTGGTTAGAGACATGACCCTGCCGTGTACCAGCTGAGAGACTCTAAATCCAGTGACAAGTCTG |
| TC13P1_pool1_33 | 893 | GAGATCATGTCCTAACCCTGATCCTCTTGTCCCACAGATATCCAGAACCCTCTAAGAAGTTCCTGCTAATATTAGTCAATGAAT |
| | | GTGGTTAGAGACATGACCCTGCCGTGTACCAGCTGAGAGACTCTAAATCCAGTGACAAGTCTG |
| TC13P1_pool1_34 | 894 | GAGATCATGTCCTAACCCTGATCCTCTTGTCCCACAGATATCCAGAACCCTCTAAGAAGTTCCTGCTTTATAAAGTCACTGAAT |
| | | GTGGTTAGAGACATGACCCTGCCGTGTACCAGCTGAGAGACTCTAAATCCAGTGACAAGTCTG |
| TC13P1_pool2_1 | 895 | GAGATCATGTCCTAACCCTGATCCTCTTGTCCCACAGATATCCAGAACCCTCTAAGAAGTTCCTGCTTTAATAAGTCAGAGAA |
| | | TGTGGTTAGAGACATGACCCTGCCGTGTACCAGCTGAGAGACTCTAAATCCAGTGACAAGTCTG |
| TC13P1_pool2_2 | 896 | GAGATCATGTCCTAACCCTGATCCTCTTGTCCCACAGATATCCAGAACCCTCTAAGAAGTTCCTGCTTAATTTAGTCATTGAAT |
| | | GTGGTTAGAGACATGACCCTGCCGTGTACCAGCTGAGAGACTCTAAATCCAGTGACAAGTCTG |
| TC13P1_pool2_3 | 897 | GAGATCATGTCCTAACCCTGATCCTCTTGTCCCACAGATATCCAGAACCCTCTAAGAAGTTCCTGCTAATTTTAGTCCAAGAAT |
| | | GTGGTTAGAGACATGACCCTGCCGTGTACCAGCTGAGAGACTCTAAATCCAGTGACAAGTCTG |
| TC13P1_pool2_4 | 898 | GAGATCATGTCCTAACCCTGATCCTCTTGTCCCACAGATATCCAGAACCCTCTAAGAAGTTCCTGCTTTATATAGTCCCAGAA |
| | | TGTGGTTAGAGACATGACCCTGCCGTGTACCAGCTGAGAGACTCTAAATCCAGTGACAAGTCTG |
| TC13P1_pool2_5 | 899 | GAGATCATGTCCTAACCCTGATCCTCTTGTCCCACAGATATCCAGAACCCTCTAAGAAGTTCCTGCTAATTATAGTCCGTGAA |
| | | TGTGGTTAGAGACATGACCCTGCCGTGTACCAGCTGAGAGACTCTAAATCCAGTGACAAGTCTG |
| TC13P1_pool2_6 | 900 | GAGATCATGTCCTAACCCTGATCCTCTTGTCCCACAGATATCCAGAACCCTCTAAGAAGTTCCTGCTTTTTAAAGTCCTAGAAT |
| | | GTGGTTAGAGACATGACCCTGCCGTGTACCAGCTGAGAGACTCTAAATCCAGTGACAAGTCTG |
| TC13P1_pool2_7 | 901 | GAGATCATGTCCTAACCCTGATCCTCTTGTCCCACAGATATCCAGAACCCTCTAAGAAGTTCCTGCTATTTATAGTCGAAGAA |
| | | TGTGGTTAGAGACATGACCCTGCCGTGTACCAGCTGAGAGACTCTAAATCCAGTGACAAGTCTG |
| TC13P1_pool2_8 | 902 | GAGATCATGTCCTAACCCTGATCCTCTTGTCCCACAGATATCCAGAACCCTCTAAGAAGTTCCTGCTAAATTAAGTCGCTGAA |
| | | TGTGGTTAGAGACATGACCCTGCCGTGTACCAGCTGAGAGACTCTAAATCCAGTGACAAGTCTG |
| TC13P1_pool2_9 | 903 | GAGATCATGTCCTAACCCTGATCCTCTTGTCCCACAGATATCCAGAACCCTCTAAGAAGTTCCTGCTATAAAAAGTCGGAGAA |
| | | TGTGGTTAGAGACATGACCCTGCCGTGTACCAGCTGAGAGACTCTAAATCCAGTGACAAGTCTG |
| TC13P1_pool2_10 | 904 | GAGATCATGTCCTAACCCTGATCCTCTTGTCCCACAGATATCCAGAACCCTCTAAGAAGTTCCTGCTTATAATAGTCGTTGAAT |
| | | GTGGTTAGAGACATGACCCTGCCGTGTACCAGCTGAGAGACTCTAAATCCAGTGACAAGTCTG |
| TC13P1_pool2_11 | 905 | GAGATCATGTCCTAACCCTGATCCTCTTGTCCCACAGATATCCAGAACCCTCTAAGAAGTTCCTGCTAAAAATAGTCTATGAAT |
| | | GTGGTTAGAGACATGACCCTGCCGTGTACCAGCTGAGAGACTCTAAATCCAGTGACAAGTCTG |
| TC13P1_pool2_12 | 906 | GAGATCATGTCCTAACCCTGATCCTCTTGTCCCACAGATATCCAGAACCCTCTAAGAAGTTCCTGCTTTTATTAGTCTCAGAAT |
| | | GTGGTTAGAGACATGACCCTGCCGTGTACCAGCTGAGAGACTCTAAATCCAGTGACAAGTCTG |
| TC13P1_pool2_13 | 907 | GAGATCATGTCCTAACCCTGATCCTCTTGTCCCACAGATATCCAGAACCCTCTAAGAAGTTCCTGCTAATAAAAGTCTGTGAA |
| | | TGTGGTTAGAGACATGACCCTGCCGTGTACCAGCTGAGAGACTCTAAATCCAGTGACAAGTCTG |
| TC13P1_pool2_14 | 908 | GAGATCATGTCCTAACCCTGATCCTCTTGTCCCACAGATATCCAGAACCCTCTAAGAAGTTCCTGCTTATTTAAGTCTTAGAAT |
| | | GTGGTTAGAGACATGACCCTGCCGTGTACCAGCTGAGAGACTCTAAATCCAGTGACAAGTCTG |
| TC13P1_pool2_15 | 909 | GAGATCATGTCCTAACCCTGATCCTCTTGTCCCACAGATATCCAGAACCCTCTAAGAAGTTCCTGCTTATTAAATTCAATGAAT |
| | | GTGGTTAGAGACATGACCCTGCCGTGTACCAGCTGAGAGACTCTAAATCCAGTGACAAGTCTG |
| TC13P1_pool2_16 | 910 | GAGATCATGTCCTAACCCTGATCCTCTTGTCCCACAGATATCCAGAACCCTCTAAGAAGTTCCTGCTTTTATAATTCACTGAAT |
| | | GTGGTTAGAGACATGACCCTGCCGTGTACCAGCTGAGAGACTCTAAATCCAGTGACAAGTCTG |
| TC13P1_pool2_17 | 911 | GAGATCATGTCCTAACCCTGATCCTCTTGTCCCACAGATATCCAGAACCCTCTAAGAAGTTCCTGCTTATTATATTCAGAGAAT |
| | | GTGGTTAGAGACATGACCCTGCCGTGTACCAGCTGAGAGACTCTAAATCCAGTGACAAGTCTG |
| TC13P1_pool2_18 | 912 | GAGATCATGTCCTAACCCTGATCCTCTTGTCCCACAGATATCCAGAACCCTCTAAGAAGTTCCTGCTATATATATTCATTGAAT |
| | | GTGGTTAGAGACATGACCCTGCCGTGTACCAGCTGAGAGACTCTAAATCCAGTGACAAGTCTG |
| TC13P1_pool2_19 | 913 | GAGATCATGTCCTAACCCTGATCCTCTTGTCCCACAGATATCCAGAACCCTCTAAGAAGTTCCTGCTATATTTATTCCAAGAAT |
| | | GTGGTTAGAGACATGACCCTGCCGTGTACCAGCTGAGAGACTCTAAATCCAGTGACAAGTCTG |
| TC13P1_pool2_20 | 914 | GAGATCATGTCCTAACCCTGATCCTCTTGTCCCACAGATATCCAGAACCCTCTAAGAAGTTCCTGCTATTATAATTCCCAGAAT |
| | | GTGGTTAGAGACATGACCCTGCCGTGTACCAGCTGAGAGACTCTAAATCCAGTGACAAGTCTG |
| TC13P1_pool2_21 | 915 | GAGATCATGTCCTAACCCTGATCCTCTTGTCCCACAGATATCCAGAACCCTCTAAGAAGTTCCTGCTAATAATATTCCGAGAA |
| | | TGTGGTTAGAGACATGACCCTGCCGTGTACCAGCTGAGAGACTCTAAATCCAGTGACAAGTCTG |
| TC13P1_pool2_22 | 916 | GAGATCATGTCCTAACCCTGATCCTCTTGTCCCACAGATATCCAGAACCCTCTAAGAAGTTCCTGCTATAATTATTCCTTGAAT |
| | | GTGGTTAGAGACATGACCCTGCCGTGTACCAGCTGAGAGACTCTAAATCCAGTGACAAGTCTG |
| TC13P1_pool2_23 | 917 | GAGATCATGTCCTAACCCTGATCCTCTTGTCCCACAGATATCCAGAACCCTCTAAGAAGTTCCTGCTAAATATATTCGAAGAAT |
| | | GTGGTTAGAGACATGACCCTGCCGTGTACCAGCTGAGAGACTCTAAATCCAGTGACAAGTCTG |
| TC13P1_pool2_24 | 918 | GAGATCATGTCCTAACCCTGATCCTCTTGTCCCACAGATATCCAGAACCCTCTAAGAAGTTCCTGCTAAAATAATTCGCAGAA |
| | | TGTGGTTAGAGACATGACCCTGCCGTGTACCAGCTGAGAGACTCTAAATCCAGTGACAAGTCTG |
| TC13P1_pool2_25 | 919 | GAGATCATGTCCTAACCCTGATCCTCTTGTCCCACAGATATCCAGAACCCTCTAAGAAGTTCCTGCTATTTTAATTCGGTGAAT |
| | | GTGGTTAGAGACATGACCCTGCCGTGTACCAGCTGAGAGACTCTAAATCCAGTGACAAGTCTG |
| TC13P1_pool2_26 | 920 | GAGATCATGTCCTAACCCTGATCCTCTTGTCCCACAGATATCCAGAACCCTCTAAGAAGTTCCTGCTTTTTATATTCGTTGAAT |
| | | GTGGTTAGAGACATGACCCTGCCGTGTACCAGCTGAGAGACTCTAAATCCAGTGACAAGTCTG |
| TC13P1_pool2_27 | 921 | GAGATCATGTCCTAACCCTGATCCTCTTGTCCCACAGATATCCAGAACCCTCTAAGAAGTTCCTGCTTAAAATATTCTAAGAAT |
| | | GTGGTTAGAGACATGACCCTGCCGTGTACCAGCTGAGAGACTCTAAATCCAGTGACAAGTCTG |
| TC13P1_pool2_28 | 922 | GAGATCATGTCCTAACCCTGATCCTCTTGTCCCACAGATATCCAGAACCCTCTAAGAAGTTCCTGCTATTAATATTCTCTGAAT |
| | | GTGGTTAGAGACATGACCCTGCCGTGTACCAGCTGAGAGACTCTAAATCCAGTGACAAGTCTG |
| TC13P1_pool2_29 | 923 | GAGATCATGTCCTAACCCTGATCCTCTTGTCCCACAGATATCCAGAACCCTCTAAGAAGTTCCTGCTTAATAAATTCTGAGAAT |
| | | GTGGTTAGAGACATGACCCTGCCGTGTACCAGCTGAGAGACTCTAAATCCAGTGACAAGTCTG |
| TC13P1_pool2_30 | 924 | GAGATCATGTCCTAACCCTGATCCTCTTGTCCCACAGATATCCAGAACCCTCTAAGAAGTTCCTGCTTAAATAATTCTTTGAAT |
| | | GTGGTTAGAGACATGACCCTGCCGTGTACCAGCTGAGAGACTCTAAATCCAGTGACAAGTCTG |
| TC13P1_pool2_31 | 925 | GAGATCATGTCCTAACCCTGATCCTCTTGTCCCACAGATATCCAGAACCCTCTAAGAAGTTCCTGCTTATAAACATCAAAGAAT |
| | | GTGGTTAGAGACATGACCCTGCCGTGTACCAGCTGAGAGACTCTAAATCCAGTGACAAGTCTG |
| TC13P1_pool2_32 | 926 | GAGATCATGTCCTAACCCTGATCCTCTTGTCCCACAGATATCCAGAACCCTCTAAGAAGTTCCTGCTTTAAATCATCACTGAAT |
| | | GTGGTTAGAGACATGACCCTGCCGTGTACCAGCTGAGAGACTCTAAATCCAGTGACAAGTCTG |
| TC13P1_pool2_33 | 927 | GAGATCATGTCCTAACCCTGATCCTCTTGTCCCACAGATATCCAGAACCCTCTAAGAAGTTCCTGCTAATATTCATCAGTGAAT |
| | | GTGGTTAGAGACATGACCCTGCCGTGTACCAGCTGAGAGACTCTAAATCCAGTGACAAGTCTG |
| TC13P1_pool2_34 | 928 | GAGATCATGTCCTAACCCTGATCCTCTTGTCCCACAGATATCCAGAACCCTCTAAGAAGTTCCTGCTTTATAACATCATTGAAT |
| | | GTGGTTAGAGACATGACCCTGCCGTGTACCAGCTGAGAGACTCTAAATCCAGTGACAAGTCTG |
| TC13P2_pool3_1 | 929 | GAGATCATGTCCTAACCCTGATCCTCTTGTCCCACAGATATCCAGAACCCTCTAAGAAGTTCCTGCTTTAATACATCCAAGAAT |
| | | GTGGTTAGAGACATGACCCTGCCGTGTACCAGCTGAGAGACTCTAAATCCAGTGACAAGTCTG |
| TC13P2_pool3_2 | 930 | GAGATCATGTCCTAACCCTGATCCTCTTGTCCCACAGATATCCAGAACCCTCTAAGAAGTTCCTGCTTAATTTCATCCCTGAAT |
| | | GTGGTTAGAGACATGACCCTGCCGTGTACCAGCTGAGAGACTCTAAATCCAGTGACAAGTCTG |
| TC13P2_pool3_3 | 931 | GAGATCATGTCCTAACCCTGATCCTCTTGTCCCACAGATATCCAGAACCCTCTAAGAAGTTCCTGCTAATTTTCATCCGAGAA |
| | | TGTGGTTAGAGACATGACCCTGCCGTGTACCAGCTGAGAGACTCTAAATCCAGTGACAAGTCTG |
| TC13P2_pool3_4 | 932 | GAGATCATGTCCTAACCCTGATCCTCTTGTCCCACAGATATCCAGAACCCTCTAAGAAGTTCCTGCTTTATATCATCCTAGAAT |
| | | GTGGTTAGAGACATGACCCTGCCGTGTACCAGCTGAGAGACTCTAAATCCAGTGACAAGTCTG |
| TC13P2_pool3_5 | 933 | GAGATCATGTCCTAACCCTGATCCTCTTGTCCCACAGATATCCAGAACCCTCTAAGAAGTTCCTGCTAATTATCATCGATGAAT |
| | | GTGGTTAGAGACATGACCCTGCCGTGTACCAGCTGAGAGACTCTAAATCCAGTGACAAGTCTG |
| TC13P2_pool3_6 | 934 | GAGATCATGTCCTAACCCTGATCCTCTTGTCCCACAGATATCCAGAACCCTCTAAGAAGTTCCTGCTTTTTAACATCGCAGAA |
| | | TGTGGTTAGAGACATGACCCTGCCGTGTACCAGCTGAGAGACTCTAAATCCAGTGACAAGTCTG |
| TC13P2_pool3_7 | 935 | GAGATCATGTCCTAACCCTGATCCTCTTGTCCCACAGATATCCAGAACCCTCTAAGAAGTTCCTGCTATTTATCATCGGAGAA |
| | | TGTGGTTAGAGACATGACCCTGCCGTGTACCAGCTGAGAGACTCTAAATCCAGTGACAAGTCTG |
| TC13P2_pool3_8 | 936 | GAGATCATGTCCTAACCCTGATCCTCTTGTCCCACAGATATCCAGAACCCTCTAAGAAGTTCCTGCTAAATTACATCGTTGAAT |
| | | GTGGTTAGAGACATGACCCTGCCGTGTACCAGCTGAGAGACTCTAAATCCAGTGACAAGTCTG |
| TC13P2_pool3_9 | 937 | GAGATCATGTCCTAACCCTGATCCTCTTGTCCCACAGATATCCAGAACCCTCTAAGAAGTTCCTGCTATAAAACATCTAAGAAT |
| | | GTGGTTAGAGACATGACCCTGCCGTGTACCAGCTGAGAGACTCTAAATCCAGTGACAAGTCTG |
| TC13P2_pool3_10 | 938 | GAGATCATGTCCTAACCCTGATCCTCTTGTCCCACAGATATCCAGAACCCTCTAAGAAGTTCCTGCTTATAATCATCTCTGAAT |
| | | GTGGTTAGAGACATGACCCTGCCGTGTACCAGCTGAGAGACTCTAAATCCAGTGACAAGTCTG |
| TC13P2_pool3_11 | 939 | GAGATCATGTCCTAACCCTGATCCTCTTGTCCCACAGATATCCAGAACCCTCTAAGAAGTTCCTGCTAAAAATCATCTGTGAA |
| | | TGTGGTTAGAGACATGACCCTGCCGTGTACCAGCTGAGAGACTCTAAATCCAGTGACAAGTCTG |
| TC13P2_pool3_12 | 940 | GAGATCATGTCCTAACCCTGATCCTCTTGTCCCACAGATATCCAGAACCCTCTAAGAAGTTCCTGCTTTTATTCATCTTAGAAT |
| | | GTGGTTAGAGACATGACCCTGCCGTGTACCAGCTGAGAGACTCTAAATCCAGTGACAAGTCTG |
| TC13P2_pool3_13 | 941 | GAGATCATGTCCTAACCCTGATCCTCTTGTCCCACAGATATCCAGAACCCTCTAAGAAGTTCCTGCTAATAAACCTCAATGAA |
| | | TGTGGTTAGAGACATGACCCTGCCGTGTACCAGCTGAGAGACTCTAAATCCAGTGACAAGTCTG |
| TC13P2_pool3_14 | 942 | GAGATCATGTCCTAACCCTGATCCTCTTGTCCCACAGATATCCAGAACCCTCTAAGAAGTTCCTGCTTATTTACCTCACAGAAT |
| | | GTGGTTAGAGACATGACCCTGCCGTGTACCAGCTGAGAGACTCTAAATCCAGTGACAAGTCTG |
| TC13P2_pool3_15 | 943 | GAGATCATGTCCTAACCCTGATCCTCTTGTCCCACAGATATCCAGAACCCTCTAAGAAGTTCCTGCTTATTAACCTCAGTGAA |
| | | TGTGGTTAGAGACATGACCCTGCCGTGTACCAGCTGAGAGACTCTAAATCCAGTGACAAGTCTG |
| TC13P2_pool3_16 | 944 | GAGATCATGTCCTAACCCTGATCCTCTTGTCCCACAGATATCCAGAACCCTCTAAGAAGTTCCTGCTTTTATACCTCATTGAAT |
| | | GTGGTTAGAGACATGACCCTGCCGTGTACCAGCTGAGAGACTCTAAATCCAGTGACAAGTCTG |
| TC13P2_pool3_17 | 945 | GAGATCATGTCCTAACCCTGATCCTCTTGTCCCACAGATATCCAGAACCCTCTAAGAAGTTCCTGCTTATTATCCTCCAAGAAT |
| | | GTGGTTAGAGACATGACCCTGCCGTGTACCAGCTGAGAGACTCTAAATCCAGTGACAAGTCTG |
| TC13P2_pool3_18 | 946 | GAGATCATGTCCTAACCCTGATCCTCTTGTCCCACAGATATCCAGAACCCTCTAAGAAGTTCCTGCTATATATCCTCCCTGAA |
| | | TGTGGTTAGAGACATGACCCTGCCGTGTACCAGCTGAGAGACTCTAAATCCAGTGACAAGTCTG |
| TC13P2_pool3_19 | 947 | GAGATCATGTCCTAACCCTGATCCTCTTGTCCCACAGATATCCAGAACCCTCTAAGAAGTTCCTGCTATATTTCCTCCGAGAA |
| | | TGTGGTTAGAGACATGACCCTGCCGTGTACCAGCTGAGAGACTCTAAATCCAGTGACAAGTCTG |
| TC13P2_pool3_20 | 948 | GAGATCATGTCCTAACCCTGATCCTCTTGTCCCACAGATATCCAGAACCCTCTAAGAAGTTCCTGCTATTATACCTCCTAGAAT |
| | | GTGGTTAGAGACATGACCCTGCCGTGTACCAGCTGAGAGACTCTAAATCCAGTGACAAGTCTG |
| TC13P2_pool3_21 | 949 | GAGATCATGTCCTAACCCTGATCCTCTTGTCCCACAGATATCCAGAACCCTCTAAGAAGTTCCTGCTAATAATCCTOGAAGAA |
| | | TGTGGTTAGAGACATGACCCTGCCGTGTACCAGCTGAGAGACTCTAAATCCAGTGACAAGTCTG |
| TC13P2_pool3_22 | 950 | GAGATCATGTCCTAACCCTGATCCTCTTGTCCCACAGATATCCAGAACCCTCTAAGAAGTTCCTGCTATAATTCCTCGCTGAA |
| | | TGTGGTTAGAGACATGACCCTGCCGTGTACCAGCTGAGAGACTCTAAATCCAGTGACAAGTCTG |
| TC13P2_pool3_23 | 951 | GAGATCATGTCCTAACCCTGATCCTCTTGTCCCACAGATATCCAGAACCCTCTAAGAAGTTCCTGCTAAATATCCTCGGAGAA |
| | | TGTGGTTAGAGACATGACCCTGCCGTGTACCAGCTGAGAGACTCTAAATCCAGTGACAAGTCTG |
| TC13P2_pool3_24 | 952 | GAGATCATGTCCTAACCCTGATCCTCTTGTCCCACAGATATCCAGAACCCTCTAAGAAGTTCCTGCTAAAATACCTCGTAGAA |
| | | TGTGGTTAGAGACATGACCCTGCCGTGTACCAGCTGAGAGACTCTAAATCCAGTGACAAGTCTG |
| TC13P2_pool3_25 | 953 | GAGATCATGTCCTAACCCTGATCCTCTTGTCCCACAGATATCCAGAACCCTCTAAGAAGTTCCTGCTATTTTACCTCTATGAAT |
| | | GTGGTTAGAGACATGACCCTGCCGTGTACCAGCTGAGAGACTCTAAATCCAGTGACAAGTCTG |
| TC13P2_pool3_26 | 954 | GAGATCATGTCCTAACCCTGATCCTCTTGTCCCACAGATATCCAGAACCCTCTAAGAAGTTCCTGCTTTTTATCCTCTCTGAAT |
| | | GTGGTTAGAGACATGACCCTGCCGTGTACCAGCTGAGAGACTCTAAATCCAGTGACAAGTCTG |
| TC13P2_pool3_27 | 955 | GAGATCATGTCCTAACCCTGATCCTCTTGTCCCACAGATATCCAGAACCCTCTAAGAAGTTCCTGCTTAAAATCCTCTGAGAA |
| | | TGTGGTTAGAGACATGACCCTGCCGTGTACCAGCTGAGAGACTCTAAATCCAGTGACAAGTCTG |
| TC13P2_pool3_28 | 956 | GAGATCATGTCCTAACCCTGATCCTCTTGTCCCACAGATATCCAGAACCCTCTAAGAAGTTCCTGCTATTAATCCTCTTTGAAT |
| | | GTGGTTAGAGACATGACCCTGCCGTGTACCAGCTGAGAGACTCTAAATCCAGTGACAAGTCTG |
| TC13P2_pool3_29 | 957 | GAGATCATGTCCTAACCCTGATCCTCTTGTCCCACAGATATCCAGAACCCTCTAAGAAGTTCCTGCTTAATAACGTCAAAGAA |
| | | TGTGGTTAGAGACATGACCCTGCCGTGTACCAGCTGAGAGACTCTAAATCCAGTGACAAGTCTG |
| TC13P2_pool3_30 | 958 | GAGATCATGTCCTAACCCTGATCCTCTTGTCCCACAGATATCCAGAACCCTCTAAGAAGTTCCTGCTTAAATACGTCACTGAA |
| | | TGTGGTTAGAGACATGACCCTGCCGTGTACCAGCTGAGAGACTCTAAATCCAGTGACAAGTCTG |
| TC13P2_pool3_31 | 959 | GAGATCATGTCCTAACCCTGATCCTCTTGTCCCACAGATATCCAGAACCCTCTAAGAAGTTCCTGCTTATAAACGTCAGAGAA |
| | | TGTGGTTAGAGACATGACCCTGCCGTGTACCAGCTGAGAGACTCTAAATCCAGTGACAAGTCTG |
| TC13P2_pool3_32 | 960 | GAGATCATGTCCTAACCCTGATCCTCTTGTCCCACAGATATCCAGAACCCTCTAAGAAGTTCCTGCTTTAAATCGTCATTGAAT |
| | | GTGGTTAGAGACATGACCCTGCCGTGTACCAGCTGAGAGACTCTAAATCCAGTGACAAGTCTG |
| TC13P2_pool3_33 | 961 | GAGATCATGTCCTAACCCTGATCCTCTTGTCCCACAGATATCCAGAACCCTCTAAGAAGTTCCTGCTAATATTCGTCCATGAA |
| | | TGTGGTTAGAGACATGACCCTGCCGTGTACCAGCTGAGAGACTCTAAATCCAGTGACAAGTCTG |
| TC13P2_pool3_34 | 962 | GAGATCATGTCCTAACCCTGATCCTCTTGTCCCACAGATATCCAGAACCCTCTAAGAAGTTCCTGCTTTATAACGTCCCTGAA |
| | | TGTGGTTAGAGACATGACCCTGCCGTGTACCAGCTGAGAGACTCTAAATCCAGTGACAAGTCTG |
| TC13P2_pool4_1 | 963 | GAGATCATGTCCTAACCCTGATCCTCTTGTCCCACAGATATCCAGAACCCTCTAAGAAGTTCCTGCTTTAATACGTCCGAGAA |
| | | TGTGGTTAGAGACATGACCCTGCCGTGTACCAGCTGAGAGACTCTAAATCCAGTGACAAGTCTG |
| TC13P2_pool4_2 | 964 | GAGATCATGTCCTAACCCTGATCCTCTTGTCCCACAGATATCCAGAACCCTCTAAGAAGTTCCTGCTTAATTTCGTCCTTGAAT |
| | | GTGGTTAGAGACATGACCCTGCCGTGTACCAGCTGAGAGACTCTAAATCCAGTGACAAGTCTG |
| TC13P2_pool4_3 | 965 | GAGATCATGTCCTAACCCTGATCCTCTTGTCCCACAGATATCCAGAACCCTCTAAGAAGTTCCTGCTAATTTTCGTCGAAGAA |
| | | TGTGGTTAGAGACATGACCCTGCCGTGTACCAGCTGAGAGACTCTAAATCCAGTGACAAGTCTG |
| TC13P2_pool4_4 | 966 | GAGATCATGTCCTAACCCTGATCCTCTTGTCCCACAGATATCCAGAACCCTCTAAGAAGTTCCTGCTTTATATCGTCGCAGAA |
| | | TGTGGTTAGAGACATGACCCTGCCGTGTACCAGCTGAGAGACTCTAAATCCAGTGACAAGTCTG |
| TC13P2_pool4_5 | 967 | GAGATCATGTCCTAACCCTGATCCTCTTGTCCCACAGATATCCAGAACCCTCTAAGAAGTTCCTGCTAATTATCGTCGGTGAA |
| | | TGTGGTTAGAGACATGACCCTGCCGTGTACCAGCTGAGAGACTCTAAATCCAGTGACAAGTCTG |
| TC13P2_pool4_6 | 968 | GAGATCATGTCCTAACCCTGATCCTCTTGTCCCACAGATATCCAGAACCCTCTAAGAAGTTCCTGCTTTTTAACGTCGTAGAA |
| | | TGTGGTTAGAGACATGACCCTGCCGTGTACCAGCTGAGAGACTCTAAATCCAGTGACAAGTCTG |
| TC13P2_pool4_7 | 969 | GAGATCATGTCCTAACCCTGATCCTCTTGTCCCACAGATATCCAGAACCCTCTAAGAAGTTCCTGCTATTTATCGTCTAAGAAT |
| | | GTGGTTAGAGACATGACCCTGCCGTGTACCAGCTGAGAGACTCTAAATCCAGTGACAAGTCTG |
| TC13P2_pool4_8 | 970 | GAGATCATGTCCTAACCCTGATCCTCTTGTCCCACAGATATCCAGAACCCTCTAAGAAGTTCCTGCTAAATTACGTCTCTGAA |
| | | TGTGGTTAGAGACATGACCCTGCCGTGTACCAGCTGAGAGACTCTAAATCCAGTGACAAGTCTG |
| TC13P2_pool4_9 | 971 | GAGATCATGTCCTAACCCTGATCCTCTTGTCCCACAGATATCCAGAACCCTCTAAGAAGTTCCTGCTATAAAACGTCTGAGAA |
| | | TGTGGTTAGAGACATGACCCTGCCGTGTACCAGCTGAGAGACTCTAAATCCAGTGACAAGTCTG |
| TC13P2_pool4_10 | 972 | GAGATCATGTCCTAACCCTGATCCTCTTGTCCCACAGATATCCAGAACCCTCTAAGAAGTTCCTGCTTATAATCGTCTTTGAAT |
| | | GTGGTTAGAGACATGACCCTGCCGTGTACCAGCTGAGAGACTCTAAATCCAGTGACAAGTCTG |
| TC13P2_pool4_11 | 973 | GAGATCATGTCCTAACCCTGATCCTCTTGTCCCACAGATATCCAGAACCCTCTAAGAAGTTCCTGCTAAAAATCTTCAATGAAT |
| | | GTGGTTAGAGACATGACCCTGCCGTGTACCAGCTGAGAGACTCTAAATCCAGTGACAAGTCTG |
| TC13P2_pool4_12 | 974 | GAGATCATGTCCTAACCCTGATCCTCTTGTCCCACAGATATCCAGAACCCTCTAAGAAGTTCCTGCTTTTATTCTTCACAGAAT |
| | | GTGGTTAGAGACATGACCCTGCCGTGTACCAGCTGAGAGACTCTAAATCCAGTGACAAGTCTG |
| TC13P2_pool4_13 | 975 | GAGATCATGTCCTAACCCTGATCCTCTTGTCCCACAGATATCCAGAACCCTCTAAGAAGTTCCTGCTAATAAACTTCAGTGAA |
| | | TGTGGTTAGAGACATGACCCTGCCGTGTACCAGCTGAGAGACTCTAAATCCAGTGACAAGTCTG |
| TC13P2_pool4_14 | 976 | GAGATCATGTCCTAACCCTGATCCTCTTGTCCCACAGATATCCAGAACCCTCTAAGAAGTTCCTGCTTATTTACTTCATAGAAT |
| | | GTGGTTAGAGACATGACCCTGCCGTGTACCAGCTGAGAGACTCTAAATCCAGTGACAAGTCTG |
| TC13P2_pool4_15 | 977 | GAGATCATGTCCTAACCCTGATCCTCTTGTCCCACAGATATCCAGAACCCTCTAAGAAGTTCCTGCTTATTAACTTCCATGAAT |
| | | GTGGTTAGAGACATGACCCTGCCGTGTACCAGCTGAGAGACTCTAAATCCAGTGACAAGTCTG |
| TC13P2_pool4_16 | 978 | GAGATCATGTCCTAACCCTGATCCTCTTGTCCCACAGATATCCAGAACCCTCTAAGAAGTTCCTGCTTTTATACTTCCCTGAAT |
| | | GTGGTTAGAGACATGACCCTGCCGTGTACCAGCTGAGAGACTCTAAATCCAGTGACAAGTCTG |
| TC13P2_pool4_17 | 979 | GAGATCATGTCCTAACCCTGATCCTCTTGTCCCACAGATATCCAGAACCCTCTAAGAAGTTCCTGCTTATTATOTTCCGAGAAT |
| | | GTGGTTAGAGACATGACCCTGCCGTGTACCAGCTGAGAGACTCTAAATCCAGTGACAAGTCTG |
| TC13P2_pool4_18 | 980 | GAGATCATGTCCTAACCCTGATCCTCTTGTCCCACAGATATCCAGAACCCTCTAAGAAGTTCCTGCTATATATCTTCCTTGAAT |
| | | GTGGTTAGAGACATGACCCTGCCGTGTACCAGCTGAGAGACTCTAAATCCAGTGACAAGTCTG |
| TC13P2_pool4_19 | 981 | GAGATCATGTCCTAACCCTGATCCTCTTGTCCCACAGATATCCAGAACCCTCTAAGAAGTTCCTGCTATATTTCTTCGAAGAAT |
| | | GTGGTTAGAGACATGACCCTGCCGTGTACCAGCTGAGAGACTCTAAATCCAGTGACAAGTCTG |
| TC13P2_pool4_20 | 982 | GAGATCATGTCCTAACCCTGATCCTCTTGTCCCACAGATATCCAGAACCCTCTAAGAAGTTCCTGCTATTATACTTCGCAGAA |
| | | TGTGGTTAGAGACATGACCCTGCCGTGTACCAGCTGAGAGACTCTAAATCCAGTGACAAGTCTG |
| TC13P2_pool4_21 | 983 | GAGATCATGTCCTAACCCTGATCCTCTTGTCCCACAGATATCCAGAACCCTCTAAGAAGTTCCTGCTAATAATCTTCGGAGAA |
| | | TGTGGTTAGAGACATGACCCTGCCGTGTACCAGCTGAGAGACTCTAAATCCAGTGACAAGTCTG |
| TC13P2_pool4_22 | 984 | GAGATCATGTCCTAACCCTGATCCTCTTGTCCCACAGATATCCAGAACCCTCTAAGAAGTTCCTGCTATAATTCTTOGTTGAAT |
| | | GTGGTTAGAGACATGACCCTGCCGTGTACCAGCTGAGAGACTCTAAATCCAGTGACAAGTCTG |
| TC13P2_pool4_23 | 985 | GAGATCATGTCCTAACCCTGATCCTCTTGTCCCACAGATATCCAGAACCCTCTAAGAAGTTCCTGCTAAATATCTTCTAAGAAT |
| | | GTGGTTAGAGACATGACCCTGCCGTGTACCAGCTGAGAGACTCTAAATCCAGTGACAAGTCTG |
| TC13P2_pool4_24 | 986 | GAGATCATGTCCTAACCCTGATCCTCTTGTCCCACAGATATCCAGAACCCTCTAAGAAGTTCCTGCTAAAATACTTCTCAGAAT |
| | | GTGGTTAGAGACATGACCCTGCCGTGTACCAGCTGAGAGACTCTAAATCCAGTGACAAGTCTG |
| TC13P2_pool4_25 | 987 | GAGATCATGTCCTAACCCTGATCCTCTTGTCCCACAGATATCCAGAACCCTCTAAGAAGTTCCTGCTATTTTACTTCTGTGAAT |
| | | GTGGTTAGAGACATGACCCTGCCGTGTACCAGCTGAGAGACTCTAAATCCAGTGACAAGTCTG |
| TC13P2_pool4_26 | 988 | GAGATCATGTCCTAACCCTGATCCTCTTGTCCCACAGATATCCAGAACCCTCTAAGAAGTTCCTGCTTTTTATCTTCTTTGAAT |
| | | GTGGTTAGAGACATGACCCTGCCGTGTACCAGCTGAGAGACTCTAAATCCAGTGACAAGTCTG |
| TC13P2_pool4_27 | 989 | GAGATCATGTCCTAACCCTGATCCTCTTGTCCCACAGATATCCAGAACCCTCTAAGAAGTTCCTGCTTAAAATGATCAAAGAA |
| | | TGTGGTTAGAGACATGACCCTGCCGTGTACCAGCTGAGAGACTCTAAATCCAGTGACAAGTCTG |
| TC13P2_pool4_28 | 990 | GAGATCATGTCCTAACCCTGATCCTCTTGTCCCACAGATATCCAGAACCCTCTAAGAAGTTCCTGCTATTAATGATCACTGAAT |
| | | GTGGTTAGAGACATGACCCTGCCGTGTACCAGCTGAGAGACTCTAAATCCAGTGACAAGTCTG |
| TC13P2_pool4_29 | 991 | GAGATCATGTCCTAACCCTGATCCTCTTGTCCCACAGATATCCAGAACCCTCTAAGAAGTTCCTGCTTAATAAGATCAGAGAA |
| | | TGTGGTTAGAGACATGACCCTGCCGTGTACCAGCTGAGAGACTCTAAATCCAGTGACAAGTCTG |
| TC13P2_pool4_30 | 992 | GAGATCATGTCCTAACCCTGATCCTCTTGTCCCACAGATATCCAGAACCCTCTAAGAAGTTCCTGCTTAAATAGATCATTGAAT |
| | | GTGGTTAGAGACATGACCCTGCCGTGTACCAGCTGAGAGACTCTAAATCCAGTGACAAGTCTG |
| TC13P2_pool4_31 | 993 | GAGATCATGTCCTAACCCTGATCCTCTTGTCCCACAGATATCCAGAACCCTCTAAGAAGTTCCTGCTTATAAAGATCCAAGAA |
| | | TGTGGTTAGAGACATGACCCTGCCGTGTACCAGCTGAGAGACTCTAAATCCAGTGACAAGTCTG |
| TC13P2_pool4_32 | 994 | GAGATCATGTCCTAACCCTGATCCTCTTGTCCCACAGATATCCAGAACCCTCTAAGAAGTTCCTGCTTTAAATGATCCCTGAA |
| | | TGTGGTTAGAGACATGACCCTGCCGTGTACCAGCTGAGAGACTCTAAATCCAGTGACAAGTCTG |
| TC13P2_pool4_33 | 995 | GAGATCATGTCCTAACCCTGATCCTCTTGTCCCACAGATATCCAGAACCCTCTAAGAAGTTCCTGCTAATATTGATCCGTGAA |
| | | TGTGGTTAGAGACATGACCCTGCCGTGTACCAGCTGAGAGACTCTAAATCCAGTGACAAGTCTG |
| TC13P2_pool4_34 | 996 | GAGATCATGTCCTAACCCTGATCCTCTTGTCCCACAGATATCCAGAACCCTCTAAGAAGTTCCTGCTTTATAAGATCCTTGAAT |
| | | GTGGTTAGAGACATGACCCTGCCGTGTACCAGCTGAGAGACTCTAAATCCAGTGACAAGTCTG |
| TC13P3_pool5_1 | 997 | GAGATCATGTCCTAACCCTGATCCTCTTGTCCCACAGATATCCAGAACCCTCTAAGAAGTTCCTGCTTTAATAGATCGAAGAA |
| | | TGTGGTTAGAGACATGACCCTGCCGTGTACCAGCTGAGAGACTCTAAATCCAGTGACAAGTCTG |
| TC13P3_pool5_2 | 998 | GAGATCATGTCCTAACCCTGATCCTCTTGTCCCACAGATATCCAGAACCCTCTAAGAAGTTCCTGCTTAATTTGATCGCTGAA |
| | | TGTGGTTAGAGACATGACCCTGCCGTGTACCAGCTGAGAGACTCTAAATCCAGTGACAAGTCTG |
| TC13P3_pool5_3 | 999 | GAGATCATGTCCTAACCCTGATCCTCTTGTCCCACAGATATCCAGAACCCTCTAAGAAGTTCCTGCTAATTTTGATCGGAGAA |
| | | TGTGGTTAGAGACATGACCCTGCCGTGTACCAGCTGAGAGACTCTAAATCCAGTGACAAGTCTG |
| TC13P3_pool5_4 | 1000 | GAGATCATGTCCTAACCCTGATCCTCTTGTCCCACAGATATCCAGAACCCTCTAAGAAGTTCCTGCTTTATATGATCGTAGAAT |
| | | GTGGTTAGAGACATGACCCTGCCGTGTACCAGCTGAGAGACTCTAAATCCAGTGACAAGTCTG |
| TC13P3_pool5_5 | 1001 | GAGATCATGTCCTAACCCTGATCCTCTTGTCCCACAGATATCCAGAACCCTCTAAGAAGTTCCTGCTAATTATGATCTATGAAT |
| | | GTGGTTAGAGACATGACCCTGCCGTGTACCAGCTGAGAGACTCTAAATCCAGTGACAAGTCTG |
| TC13P3_pool5_6 | 1002 | GAGATCATGTCCTAACCCTGATCCTCTTGTCCCACAGATATCCAGAACCCTCTAAGAAGTTCCTGCTTTTTAAGATCTCAGAAT |
| | | GTGGTTAGAGACATGACCCTGCCGTGTACCAGCTGAGAGACTCTAAATCCAGTGACAAGTCTG |
| TC13P3_pool5_7 | 1003 | GAGATCATGTCCTAACCCTGATCCTCTTGTCCCACAGATATCCAGAACCCTCTAAGAAGTTCCTGCTATTTATGATCTGAGAAT |
| | | GTGGTTAGAGACATGACCCTGCCGTGTACCAGCTGAGAGACTCTAAATCCAGTGACAAGTCTG |
| TC13P3_pool5_8 | 1004 | GAGATCATGTCCTAACCCTGATCCTCTTGTCCCACAGATATCCAGAACCCTCTAAGAAGTTCCTGCTAAATTAGATCTTTGAAT |
| | | GTGGTTAGAGACATGACCCTGCCGTGTACCAGCTGAGAGACTCTAAATCCAGTGACAAGTCTG |
| TC13P3_pool5_9 | 1005 | GAGATCATGTCCTAACCCTGATCCTCTTGTCCCACAGATATCCAGAACCCTCTAAGAAGTTCCTGCTATAAAAGCTCAAAGAA |
| | | TGTGGTTAGAGACATGACCCTGCCGTGTACCAGCTGAGAGACTCTAAATCCAGTGACAAGTCTG |
| TC13P3_pool5_10 | 1006 | GAGATCATGTCCTAACCCTGATCCTCTTGTCCCACAGATATCCAGAACCCTCTAAGAAGTTCCTGCTTATAATGCTCACTGAA |
| | | TGTGGTTAGAGACATGACCCTGCCGTGTACCAGCTGAGAGACTCTAAATCCAGTGACAAGTCTG |
| TC13P3_pool5_11 | 1007 | GAGATCATGTCCTAACCCTGATCCTCTTGTCCCACAGATATCCAGAACCCTCTAAGAAGTTCCTGCTAAAAATGCTCAGTGAA |
| | | TGTGGTTAGAGACATGACCCTGCCGTGTACCAGCTGAGAGACTCTAAATCCAGTGACAAGTCTG |
| TC13P3_pool5_12 | 1008 | GAGATCATGTCCTAACCCTGATCCTCTTGTCCCACAGATATCCAGAACCCTCTAAGAAGTTCCTGCTTTTATTGCTCATAGAAT |
| | | GTGGTTAGAGACATGACCCTGCCGTGTACCAGCTGAGAGACTCTAAATCCAGTGACAAGTCTG |
| TC13P3_pool5_13 | 1009 | GAGATCATGTCCTAACCCTGATCCTCTTGTCCCACAGATATCCAGAACCCTCTAAGAAGTTCCTGCTAATAAAGCTCCATGAA |
| | | TGTGGTTAGAGACATGACCCTGCCGTGTACCAGCTGAGAGACTCTAAATCCAGTGACAAGTCTG |
| TC13P3_pool5_14 | 1010 | GAGATCATGTCCTAACCCTGATCCTCTTGTCCCACAGATATCCAGAACCCTCTAAGAAGTTCCTGCTTATTTAGCTCCCAGAA |
| | | TGTGGTTAGAGACATGACCCTGCCGTGTACCAGCTGAGAGACTCTAAATCCAGTGACAAGTCTG |
| TC13P3_pool5_15 | 1011 | GAGATCATGTCCTAACCCTGATCCTCTTGTCCCACAGATATCCAGAACCCTCTAAGAAGTTCCTGCTTATTAAGCTCCGTGAA |
| | | TGTGGTTAGAGACATGACCCTGCCGTGTACCAGCTGAGAGACTCTAAATCCAGTGACAAGTCTG |
| TC13P3_pool5_16 | 1012 | GAGATCATGTCCTAACCCTGATCCTCTTGTCCCACAGATATCCAGAACCCTCTAAGAAGTTCCTGCTTTTATAGCTCCTTGAAT |
| | | GTGGTTAGAGACATGACCCTGCCGTGTACCAGCTGAGAGACTCTAAATCCAGTGACAAGTCTG |
| TC13P3_pool5_17 | 1013 | GAGATCATGTCCTAACCCTGATCCTCTTGTCCCACAGATATCCAGAACCCTCTAAGAAGTTCCTGCTTATTATGCTCGAAGAA |
| | | TGTGGTTAGAGACATGACCCTGCCGTGTACCAGCTGAGAGACTCTAAATCCAGTGACAAGTCTG |
| TC13P3_pool5_18 | 1014 | GAGATCATGTCCTAACCCTGATCCTCTTGTCCCACAGATATCCAGAACCCTCTAAGAAGTTCCTGCTATATATGCTCGCTGAA |
| | | TGTGGTTAGAGACATGACCCTGCCGTGTACCAGCTGAGAGACTCTAAATCCAGTGACAAGTCTG |
| TC13P3_pool5_19 | 1015 | GAGATCATGTCCTAACCCTGATCCTCTTGTCCCACAGATATCCAGAACCCTCTAAGAAGTTCCTGCTATATTTGCTCGGAGAA |
| | | TGTGGTTAGAGACATGACCCTGCCGTGTACCAGCTGAGAGACTCTAAATCCAGTGACAAGTCTG |
| TC13P3_pool5_20 | 1016 | GAGATCATGTCCTAACCCTGATCCTCTTGTCCCACAGATATCCAGAACCCTCTAAGAAGTTCCTGCTATTATAGCTCGTAGAA |
| | | TGTGGTTAGAGACATGACCCTGCCGTGTACCAGCTGAGAGACTCTAAATCCAGTGACAAGTCTG |
| TC13P3_pool5_21 | 1017 | GAGATCATGTCCTAACCCTGATCCTCTTGTCCCACAGATATCCAGAACCCTCTAAGAAGTTCCTGCTAATAATGCTCTAAGAA |
| | | TGTGGTTAGAGACATGACCCTGCCGTGTACCAGCTGAGAGACTCTAAATCCAGTGACAAGTCTG |
| TC13P3_pool5_22 | 1018 | GAGATCATGTCCTAACCCTGATCCTCTTGTCCCACAGATATCCAGAACCCTCTAAGAAGTTCCTGCTATAATTGCTCTCTGAAT |
| | | GTGGTTAGAGACATGACCCTGCCGTGTACCAGCTGAGAGACTCTAAATCCAGTGACAAGTCTG |
| TC13P3_pool5_23 | 1019 | GAGATCATGTCCTAACCCTGATCCTCTTGTCCCACAGATATCCAGAACCCTCTAAGAAGTTCCTGCTAAATATGCTCTGAGAA |
| | | TGTGGTTAGAGACATGACCCTGCCGTGTACCAGCTGAGAGACTCTAAATCCAGTGACAAGTCTG |
| TC13P3_pool5_24 | 1020 | GAGATCATGTCCTAACCCTGATCCTCTTGTCCCACAGATATCCAGAACCCTCTAAGAAGTTCCTGCTAAAATAGCTCTTAGAA |
| | | TGTGGTTAGAGACATGACCCTGCCGTGTACCAGCTGAGAGACTCTAAATCCAGTGACAAGTCTG |
| TC13P3_pool5_25 | 1021 | GAGATCATGTCCTAACCCTGATCCTCTTGTCCCACAGATATCCAGAACCCTCTAAGAAGTTCCTGCTATTTTAGGTCAATGAAT |
| | | GTGGTTAGAGACATGACCCTGCCGTGTACCAGCTGAGAGACTCTAAATCCAGTGACAAGTCTG |
| TC13P3_pool5_26 | 1022 | GAGATCATGTCCTAACCCTGATCCTCTTGTCCCACAGATATCCAGAACCCTCTAAGAAGTTCCTGCTTTTTATGGTCACTGAAT |
| | | GTGGTTAGAGACATGACCCTGCCGTGTACCAGCTGAGAGACTCTAAATCCAGTGACAAGTCTG |
| TC13P3_pool5_27 | 1023 | GAGATCATGTCCTAACCCTGATCCTCTTGTCCCACAGATATCCAGAACCCTCTAAGAAGTTCCTGCTTAAAATGGTCAGAGAA |
| | | TGTGGTTAGAGACATGACCCTGCCGTGTACCAGCTGAGAGACTCTAAATCCAGTGACAAGTCTG |
| TC13P3_pool5_28 | 1024 | GAGATCATGTCCTAACCCTGATCCTCTTGTCCCACAGATATCCAGAACCCTCTAAGAAGTTCCTGCTATTAATGGTCATTGAAT |
| | | GTGGTTAGAGACATGACCCTGCCGTGTACCAGCTGAGAGACTCTAAATCCAGTGACAAGTCTG |
| TC13P3_pool5_29 | 1025 | GAGATCATGTCCTAACCCTGATCCTCTTGTCCCACAGATATCCAGAACCCTCTAAGAAGTTCCTGCTTAATAAGGTCCAAGAA |
| | | TGTGGTTAGAGACATGACCCTGCCGTGTACCAGCTGAGAGACTCTAAATCCAGTGACAAGTCTG |
| TC13P3_pool5_30 | 1026 | GAGATCATGTCCTAACCCTGATCCTCTTGTCCCACAGATATCCAGAACCCTCTAAGAAGTTCCTGCTTAAATAGGTCCCTGAA |
| | | TGTGGTTAGAGACATGACCCTGCCGTGTACCAGCTGAGAGACTCTAAATCCAGTGACAAGTCTG |
| TC13P3_pool5_31 | 1027 | GAGATCATGTCCTAACCCTGATCCTCTTGTCCCACAGATATCCAGAACCCTCTAAGAAGTTCCTGCTTATAAAGGTCCGAGAA |
| | | TGTGGTTAGAGACATGACCCTGCCGTGTACCAGCTGAGAGACTCTAAATCCAGTGACAAGTCTG |
| TC13P3_pool5_32 | 1028 | GAGATCATGTCCTAACCCTGATCCTCTTGTCCCACAGATATCCAGAACCCTCTAAGAAGTTCCTGCTTTAAATGGTCCTTGAA |
| | | TGTGGTTAGAGACATGACCCTGCCGTGTACCAGCTGAGAGACTCTAAATCCAGTGACAAGTCTG |
| TC13P3_pool5_33 | 1029 | GAGATCATGTCCTAACCCTGATCCTCTTGTCCCACAGATATCCAGAACCCTCTAAGAAGTTCCTGCTAATATTGGTCGATGAA |
| | | TGTGGTTAGAGACATGACCCTGCCGTGTACCAGCTGAGAGACTCTAAATCCAGTGACAAGTCTG |
| TC13P3_pool5_34 | 1030 | GAGATCATGTCCTAACCCTGATCCTCTTGTCCCACAGATATCCAGAACCCTCTAAGAAGTTCCTGCTTTATAAGGTCGCTGAA |
| | | TGTGGTTAGAGACATGACCCTGCCGTGTACCAGCTGAGAGACTCTAAATCCAGTGACAAGTCTG |
| TC13P3_pool6_1 | 1031 | GAGATCATGTCCTAACCCTGATCCTCTTGTCCCACAGATATCCAGAACCCTCTAAGAAGTTCCTGCTTTAATAGGTCGGAGAA |
| | | TGTGGTTAGAGACATGACCCTGCCGTGTACCAGCTGAGAGACTCTAAATCCAGTGACAAGTCTG |
| TC13P3_pool6_2 | 1032 | GAGATCATGTCCTAACCCTGATCCTCTTGTCCCACAGATATCCAGAACCCTCTAAGAAGTTCCTGCTTAATTTGGTCGTTGAA |
| | | TGTGGTTAGAGACATGACCCTGCCGTGTACCAGCTGAGAGACTCTAAATCCAGTGACAAGTCTG |
| TC13P3_pool6_3 | 1033 | GAGATCATGTCCTAACCCTGATCCTCTTGTCCCACAGATATCCAGAACCCTCTAAGAAGTTCCTGCTAATTTTGGTCTAAGAAT |
| | | GTGGTTAGAGACATGACCCTGCCGTGTACCAGCTGAGAGACTCTAAATCCAGTGACAAGTCTG |
| TC13P3_pool6_4 | 1034 | GAGATCATGTCCTAACCCTGATCCTCTTGTCCCACAGATATCCAGAACCCTCTAAGAAGTTCCTGCTTTATATGGTCTCAGAA |
| | | TGTGGTTAGAGACATGACCCTGCCGTGTACCAGCTGAGAGACTCTAAATCCAGTGACAAGTCTG |
| TC13P3_pool6_5 | 1035 | GAGATCATGTCCTAACCCTGATCCTCTTGTCCCACAGATATCCAGAACCCTCTAAGAAGTTCCTGCTAATTATGGTCTGTGAA |
| | | TGTGGTTAGAGACATGACCCTGCCGTGTACCAGCTGAGAGACTCTAAATCCAGTGACAAGTCTG |
| TC13P3_pool6_6 | 1036 | GAGATCATGTCCTAACCCTGATCCTCTTGTCCCACAGATATCCAGAACCCTCTAAGAAGTTCCTGCTTTTTAAGGTCTTAGAAT |
| | | GTGGTTAGAGACATGACCCTGCCGTGTACCAGCTGAGAGACTCTAAATCCAGTGACAAGTCTG |
| TC13P3_pool6_7 | 1037 | GAGATCATGTCCTAACCCTGATCCTCTTGTCCCACAGATATCCAGAACCCTCTAAGAAGTTCCTGCTATTTATGTTCAAAGAAT |
| | | GTGGTTAGAGACATGACCCTGCCGTGTACCAGCTGAGAGACTCTAAATCCAGTGACAAGTCTG |
| TC13P3_pool6_8 | 1038 | GAGATCATGTCCTAACCCTGATCCTCTTGTCCCACAGATATCCAGAACCCTCTAAGAAGTTCCTGCTAAATTAGTTCACTGAAT |
| | | GTGGTTAGAGACATGACCCTGCCGTGTACCAGCTGAGAGACTCTAAATCCAGTGACAAGTCTG |
| TC13P3_pool6_9 | 1039 | GAGATCATGTCCTAACCCTGATCCTCTTGTCCCACAGATATCCAGAACCCTCTAAGAAGTTCCTGCTATAAAAGTTCAGAGAA |
| | | TGTGGTTAGAGACATGACCCTGCCGTGTACCAGCTGAGAGACTCTAAATCCAGTGACAAGTCTG |
| TC13P3_pool6_10 | 1040 | GAGATCATGTCCTAACCCTGATCCTCTTGTCCCACAGATATCCAGAACCCTCTAAGAAGTTCCTGCTTATAATGTTCATTGAAT |
| | | GTGGTTAGAGACATGACCCTGCCGTGTACCAGCTGAGAGACTCTAAATCCAGTGACAAGTCTG |
| TC13P3_pool6_11 | 1041 | GAGATCATGTCCTAACCCTGATCCTCTTGTCCCACAGATATCCAGAACCCTCTAAGAAGTTCCTGCTAAAAATGTTCCATGAA |
| | | TGTGGTTAGAGACATGACCCTGCCGTGTACCAGCTGAGAGACTCTAAATCCAGTGACAAGTCTG |
| TC13P3_pool6_12 | 1042 | GAGATCATGTCCTAACCCTGATCCTCTTGTCCCACAGATATCCAGAACCCTCTAAGAAGTTCCTGCTTTTATTGTTCCCAGAAT |
| | | GTGGTTAGAGACATGACCCTGCCGTGTACCAGCTGAGAGACTCTAAATCCAGTGACAAGTCTG |
| TC13P3_pool6_13 | 1043 | GAGATCATGTCCTAACCCTGATCCTCTTGTCCCACAGATATCCAGAACCCTCTAAGAAGTTCCTGCTAATAAAGTTCCGTGAA |
| | | TGTGGTTAGAGACATGACCCTGCCGTGTACCAGCTGAGAGACTCTAAATCCAGTGACAAGTCTG |
| TC13P3_pool6_14 | 1044 | GAGATCATGTCCTAACCCTGATCCTCTTGTCCCACAGATATCCAGAACCCTCTAAGAAGTTCCTGCTTATTTAGTTCCTAGAAT |
| | | GTGGTTAGAGACATGACCCTGCCGTGTACCAGCTGAGAGACTCTAAATCCAGTGACAAGTCTG |
| TC13P3_pool6_15 | 1045 | GAGATCATGTCCTAACCCTGATCCTCTTGTCCCACAGATATCCAGAACCCTCTAAGAAGTTCCTGCTTATTAAGTTCGATGAAT |
| | | GTGGTTAGAGACATGACCCTGCCGTGTACCAGCTGAGAGACTCTAAATCCAGTGACAAGTCTG |
| TC13P3_pool6_16 | 1046 | GAGATCATGTCCTAACCCTGATCCTCTTGTCCCACAGATATCCAGAACCCTCTAAGAAGTTCCTGCTTTTATAGTTCGCTGAAT |
| | | GTGGTTAGAGACATGACCCTGCCGTGTACCAGCTGAGAGACTCTAAATCCAGTGACAAGTCTG |
| TC13P3_pool6_17 | 1047 | GAGATCATGTCCTAACCCTGATCCTCTTGTCCCACAGATATCCAGAACCCTCTAAGAAGTTCCTGCTTATTATGTTCGGAGAA |
| | | TGTGGTTAGAGACATGACCCTGCCGTGTACCAGCTGAGAGACTCTAAATCCAGTGACAAGTCTG |
| TC13P3_pool6_18 | 1048 | GAGATCATGTCCTAACCCTGATCCTCTTGTCCCACAGATATCCAGAACCCTCTAAGAAGTTCCTGCTATATATGTTCGTTGAAT |
| | | GTGGTTAGAGACATGACCCTGCCGTGTACCAGCTGAGAGACTCTAAATCCAGTGACAAGTCTG |
| TC13P3_pool6_19 | 1049 | GAGATCATGTCCTAACCCTGATCCTCTTGTCCCACAGATATCCAGAACCCTCTAAGAAGTTCCTGCTATATTTGTTCTAAGAAT |
| | | GTGGTTAGAGACATGACCCTGCCGTGTACCAGCTGAGAGACTCTAAATCCAGTGACAAGTCTG |
| TC13P3_pool6_20 | 1050 | GAGATCATGTCCTAACCCTGATCCTCTTGTCCCACAGATATCCAGAACCCTCTAAGAAGTTCCTGCTATTATAGTTCTCAGAAT |
| | | GTGGTTAGAGACATGACCCTGCCGTGTACCAGCTGAGAGACTCTAAATCCAGTGACAAGTCTG |
| TC13P3_pool6_21 | 1051 | GAGATCATGTCCTAACCCTGATCCTCTTGTCCCACAGATATCCAGAACCCTCTAAGAAGTTCCTGCTAATAATGTTCTGAGAA |
| | | TGTGGTTAGAGACATGACCCTGCCGTGTACCAGCTGAGAGACTCTAAATCCAGTGACAAGTCTG |
| TC13P3_pool6_22 | 1052 | GAGATCATGTCCTAACCCTGATCCTCTTGTCCCACAGATATCCAGAACCCTCTAAGAAGTTCCTGCTATAATTGTTCTTTGAAT |
| | | GTGGTTAGAGACATGACCCTGCCGTGTACCAGCTGAGAGACTCTAAATCCAGTGACAAGTCTG |
| TC13P3_pool6_23 | 1053 | GAGATCATGTCCTAACCCTGATCCTCTTGTCCCACAGATATCCAGAACCCTCTAAGAAGTTCCTGCTAAATATTATCAAAGAAT |
| | | GTGGTTAGAGACATGACCCTGCCGTGTACCAGCTGAGAGACTCTAAATCCAGTGACAAGTCTG |
| TC13P3_pool6_24 | 1054 | GAGATCATGTCCTAACCCTGATCCTCTTGTCCCACAGATATCCAGAACCCTCTAAGAAGTTCCTGCTAAAATATATCACAGAAT |
| | | GTGGTTAGAGACATGACCCTGCCGTGTACCAGCTGAGAGACTCTAAATCCAGTGACAAGTCTG |
| TC13P3_pool6_25 | 1055 | GAGATCATGTCCTAACCCTGATCCTCTTGTCCCACAGATATCCAGAACCCTCTAAGAAGTTCCTGCTATTTTATATCAGTGAAT |
| | | GTGGTTAGAGACATGACCCTGCCGTGTACCAGCTGAGAGACTCTAAATCCAGTGACAAGTCTG |
| TC13P3_pool6_26 | 1056 | GAGATCATGTCCTAACCCTGATCCTCTTGTCCCACAGATATCCAGAACCCTCTAAGAAGTTCCTGCTTTTTATTATCATTGAAT |
| | | GTGGTTAGAGACATGACCCTGCCGTGTACCAGCTGAGAGACTCTAAATCCAGTGACAAGTCTG |
| TC13P3_pool6_27 | 1057 | GAGATCATGTCCTAACCCTGATCCTCTTGTCCCACAGATATCCAGAACCCTCTAAGAAGTTCCTGCTTAAAATTATCCAAGAAT |
| | | GTGGTTAGAGACATGACCCTGCCGTGTACCAGCTGAGAGACTCTAAATCCAGTGACAAGTCTG |
| TC13P3_pool6_28 | 1058 | GAGATCATGTCCTAACCCTGATCCTCTTGTCCCACAGATATCCAGAACCCTCTAAGAAGTTCCTGCTATTAATTATCCCTGAAT |
| | | GTGGTTAGAGACATGACCCTGCCGTGTACCAGCTGAGAGACTCTAAATCCAGTGACAAGTCTG |
| TC13P3_pool6_29 | 1059 | GAGATCATGTCCTAACCCTGATCCTCTTGTCCCACAGATATCCAGAACCCTCTAAGAAGTTCCTGCTTAATAATATCCGAGAA |
| | | TGTGGTTAGAGACATGACCCTGCCGTGTACCAGCTGAGAGACTCTAAATCCAGTGACAAGTCTG |
| TC13P3_pool6_30 | 1060 | GAGATCATGTCCTAACCCTGATCCTCTTGTCCCACAGATATCCAGAACCCTCTAAGAAGTTCCTGCTTAAATATATCCTTGAAT |
| | | GTGGTTAGAGACATGACCCTGCCGTGTACCAGCTGAGAGACTCTAAATCCAGTGACAAGTCTG |
| TC13P3_pool6_31 | 1061 | GAGATCATGTCCTAACCCTGATCCTCTTGTCCCACAGATATCCAGAACCCTCTAAGAAGTTCCTGCTTATAAATATCGAAGAAT |
| | | GTGGTTAGAGACATGACCCTGCCGTGTACCAGCTGAGAGACTCTAAATCCAGTGACAAGTCTG |
| TC13P3_pool6_32 | 1062 | GAGATCATGTCCTAACCCTGATCCTCTTGTCCCACAGATATCCAGAACCCTCTAAGAAGTTCCTGCTTTAAATTATCGCTGAAT |
| | | GTGGTTAGAGACATGACCCTGCCGTGTACCAGCTGAGAGACTCTAAATCCAGTGACAAGTCTG |
| TC13P3_pool6_33 | 1063 | GAGATCATGTCCTAACCCTGATCCTCTTGTCCCACAGATATCCAGAACCCTCTAAGAAGTTCCTGCTAATATTTATCGGTGAAT |
| | | GTGGTTAGAGACATGACCCTGCCGTGTACCAGCTGAGAGACTCTAAATCCAGTGACAAGTCTG |
| TC13P3_pool6_34 | 1064 | GAGATCATGTCCTAACCCTGATCCTCTTGTCCCACAGATATCCAGAACCCTCTAAGAAGTTCCTGCTTTATAATATCGTTGAAT |
| | | GTGGTTAGAGACATGACCCTGCCGTGTACCAGCTGAGAGACTCTAAATCCAGTGACAAGTCTG |
| TC13P4_pool7_1 | 1065 | GAGATCATGTCCTAACCCTGATCCTCTTGTCCCACAGATATCCAGAACCCTCTAAGAAGTTCCTGCTTTAATATATCTAAGAAT |
| | | GTGGTTAGAGACATGACCCTGCCGTGTACCAGCTGAGAGACTCTAAATCCAGTGACAAGTCTG |
| TC13P4_pool7_2 | 1066 | GAGATCATGTCCTAACCCTGATCCTCTTGTCCCACAGATATCCAGAACCCTCTAAGAAGTTCCTGCTTAATTTTATCTCTGAAT |
| | | GTGGTTAGAGACATGACCCTGCCGTGTACCAGCTGAGAGACTCTAAATCCAGTGACAAGTCTG |
| TC13P4_pool7_3 | 1067 | GAGATCATGTCCTAACCCTGATCCTCTTGTCCCACAGATATCCAGAACCCTCTAAGAAGTTCCTGCTAATTTTTATCTGAGAAT |
| | | GTGGTTAGAGACATGACCCTGCCGTGTACCAGCTGAGAGACTCTAAATCCAGTGACAAGTCTG |
| TC13P4_pool7_4 | 1068 | GAGATCATGTCCTAACCCTGATCCTCTTGTCCCACAGATATCCAGAACCCTCTAAGAAGTTCCTGCTTTATATTATOTTAGAAT |
| | | GTGGTTAGAGACATGACCCTGCCGTGTACCAGCTGAGAGACTCTAAATCCAGTGACAAGTCTG |
| TC13P4_pool7_5 | 1069 | GAGATCATGTCCTAACCCTGATCCTCTTGTCCCACAGATATCCAGAACCCTCTAAGAAGTTCCTGCTAATTATTCTCAATGAAT |
| | | GTGGTTAGAGACATGACCCTGCCGTGTACCAGCTGAGAGACTCTAAATCCAGTGACAAGTCTG |
| TC13P4_pool7_6 | 1070 | GAGATCATGTCCTAACCCTGATCCTCTTGTCCCACAGATATCCAGAACCCTCTAAGAAGTTCCTGCTTTTTAATCTCACAGAAT |
| | | GTGGTTAGAGACATGACCCTGCCGTGTACCAGCTGAGAGACTCTAAATCCAGTGACAAGTCTG |
| TC13P4_pool7_7 | 1071 | GAGATCATGTCCTAACCCTGATCCTCTTGTCCCACAGATATCCAGAACCCTCTAAGAAGTTCCTGCTATTTATTCTCAGAGAAT |
| | | GTGGTTAGAGACATGACCCTGCCGTGTACCAGCTGAGAGACTCTAAATCCAGTGACAAGTCTG |
| TC13P4_pool7_8 | 1072 | GAGATCATGTCCTAACCCTGATCCTCTTGTCCCACAGATATCCAGAACCCTCTAAGAAGTTCCTGCTAAATTATCTCATTGAAT |
| | | GTGGTTAGAGACATGACCCTGCCGTGTACCAGCTGAGAGACTCTAAATCCAGTGACAAGTCTG |
| TC13P4_pool7_9 | 1073 | GAGATCATGTCCTAACCCTGATCCTCTTGTCCCACAGATATCCAGAACCCTCTAAGAAGTTCCTGCTATAAAATCTCCAAGAA |
| | | TGTGGTTAGAGACATGACCCTGCCGTGTACCAGCTGAGAGACTCTAAATCCAGTGACAAGTCTG |
| TC13P4_pool7_10 | 1074 | GAGATCATGTCCTAACCCTGATCCTCTTGTCCCACAGATATCCAGAACCCTCTAAGAAGTTCCTGCTTATAATTCTCCCTGAAT |
| | | GTGGTTAGAGACATGACCCTGCCGTGTACCAGCTGAGAGACTCTAAATCCAGTGACAAGTCTG |
| TC13P4_pool7_11 | 1075 | GAGATCATGTCCTAACCCTGATCCTCTTGTCCCACAGATATCCAGAACCCTCTAAGAAGTTCCTGCTAAAAATTCTCCGTGAA |
| | | TGTGGTTAGAGACATGACCCTGCCGTGTACCAGCTGAGAGACTCTAAATCCAGTGACAAGTCTG |
| TC13P4_pool7_12 | 1076 | GAGATCATGTCCTAACCCTGATCCTCTTGTCCCACAGATATCCAGAACCCTCTAAGAAGTTCCTGCTTTTATTTCTCCTAGAAT |
| | | GTGGTTAGAGACATGACCCTGCCGTGTACCAGCTGAGAGACTCTAAATCCAGTGACAAGTCTG |
| TC13P4_pool7_13 | 1077 | GAGATCATGTCCTAACCCTGATCCTCTTGTCCCACAGATATCCAGAACCCTCTAAGAAGTTCCTGCTAATAAATCTCGATGAA |
| | | TGTGGTTAGAGACATGACCCTGCCGTGTACCAGCTGAGAGACTCTAAATCCAGTGACAAGTCTG |
| TC13P4_pool7_14 | 1078 | GAGATCATGTCCTAACCCTGATCCTCTTGTCCCACAGATATCCAGAACCCTCTAAGAAGTTCCTGCTTATTTATCTCGCAGAAT |
| | | GTGGTTAGAGACATGACCCTGCCGTGTACCAGCTGAGAGACTCTAAATCCAGTGACAAGTCTG |
| TC13P4_pool7_15 | 1079 | GAGATCATGTCCTAACCCTGATCCTCTTGTCCCACAGATATCCAGAACCCTCTAAGAAGTTCCTGCTTATTAATCTCGGTGAA |
| | | TGTGGTTAGAGACATGACCCTGCCGTGTACCAGCTGAGAGACTCTAAATCCAGTGACAAGTCTG |
| TC13P4_pool7_16 | 1080 | GAGATCATGTCCTAACCCTGATCCTCTTGTCCCACAGATATCCAGAACCCTCTAAGAAGTTCCTGCTTTTATATCTCGTTGAAT |
| | | GTGGTTAGAGACATGACCCTGCCGTGTACCAGCTGAGAGACTCTAAATCCAGTGACAAGTCTG |
| TC13P4_pool7_17 | 1081 | GAGATCATGTCCTAACCCTGATCCTCTTGTCCCACAGATATCCAGAACCCTCTAAGAAGTTCCTGCTTATTATTCTCTAAGAAT |
| | | GTGGTTAGAGACATGACCCTGCCGTGTACCAGCTGAGAGACTCTAAATCCAGTGACAAGTCTG |
| TC13P4_pool7_18 | 1082 | GAGATCATGTCCTAACCCTGATCCTCTTGTCCCACAGATATCCAGAACCCTCTAAGAAGTTCCTGCTATATATTCTCTCTGAAT |
| | | GTGGTTAGAGACATGACCCTGCCGTGTACCAGCTGAGAGACTCTAAATCCAGTGACAAGTCTG |
| TC13P4_pool7_19 | 1083 | GAGATCATGTCCTAACCCTGATCCTCTTGTCCCACAGATATCCAGAACCCTCTAAGAAGTTCCTGCTATATTTTCTCTGAGAAT |
| | | GTGGTTAGAGACATGACCCTGCCGTGTACCAGCTGAGAGACTCTAAATCCAGTGACAAGTCTG |
| TC13P4_pool7_20 | 1084 | GAGATCATGTCCTAACCCTGATCCTCTTGTCCCACAGATATCCAGAACCCTCTAAGAAGTTCCTGCTATTATATCTCTTAGAAT |
| | | GTGGTTAGAGACATGACCCTGCCGTGTACCAGCTGAGAGACTCTAAATCCAGTGACAAGTCTG |
| TC13P4_pool7_21 | 1085 | GAGATCATGTCCTAACCCTGATCCTCTTGTCCCACAGATATCCAGAACCCTCTAAGAAGTTCCTGCTAATAATTGTCAAAGAAT |
| | | GTGGTTAGAGACATGACCCTGCCGTGTACCAGCTGAGAGACTCTAAATCCAGTGACAAGTCTG |
| TC13P4_pool7_22 | 1086 | GAGATCATGTCCTAACCCTGATCCTCTTGTCCCACAGATATCCAGAACCCTCTAAGAAGTTCCTGCTATAATTTGTCACTGAAT |
| | | GTGGTTAGAGACATGACCCTGCCGTGTACCAGCTGAGAGACTCTAAATCCAGTGACAAGTCTG |
| TC13P4_pool7_23 | 1087 | GAGATCATGTCCTAACCCTGATCCTCTTGTCCCACAGATATCCAGAACCCTCTAAGAAGTTCCTGCTAAATATTGTCAGAGAA |
| | | TGTGGTTAGAGACATGACCCTGCCGTGTACCAGCTGAGAGACTCTAAATCCAGTGACAAGTCTG |
| TC13P4_pool7_24 | 1088 | GAGATCATGTCCTAACCCTGATCCTCTTGTCCCACAGATATCCAGAACCCTCTAAGAAGTTCCTGCTAAAATATGTCATAGAAT |
| | | GTGGTTAGAGACATGACCCTGCCGTGTACCAGCTGAGAGACTCTAAATCCAGTGACAAGTCTG |
| TC13P4_pool7_25 | 1089 | GAGATCATGTCCTAACCCTGATCCTCTTGTCCCACAGATATCCAGAACCCTCTAAGAAGTTCCTGCTATTTTATGTCCATGAAT |
| | | GTGGTTAGAGACATGACCCTGCCGTGTACCAGCTGAGAGACTCTAAATCCAGTGACAAGTCTG |
| TC13P4_pool7_26 | 1090 | GAGATCATGTCCTAACCCTGATCCTCTTGTCCCACAGATATCCAGAACCCTCTAAGAAGTTCCTGCTTTTTATTGTCCCTGAAT |
| | | GTGGTTAGAGACATGACCCTGCCGTGTACCAGCTGAGAGACTCTAAATCCAGTGACAAGTCTG |
| TC13P4_pool7_27 | 1091 | GAGATCATGTCCTAACCCTGATCCTCTTGTCCCACAGATATCCAGAACCCTCTAAGAAGTTCCTGCTTAAAATTGTCCGAGAA |
| | | TGTGGTTAGAGACATGACCCTGCCGTGTACCAGCTGAGAGACTCTAAATCCAGTGACAAGTCTG |
| TC13P4_pool7_28 | 1092 | GAGATCATGTCCTAACCCTGATCCTCTTGTCCCACAGATATCCAGAACCCTCTAAGAAGTTCCTGCTATTAATTGTCCTTGAAT |
| | | GTGGTTAGAGACATGACCCTGCCGTGTACCAGCTGAGAGACTCTAAATCCAGTGACAAGTCTG |
| TC13P4_pool7_29 | 1093 | GAGATCATGTCCTAACCCTGATCCTCTTGTCCCACAGATATCCAGAACCCTCTAAGAAGTTCCTGCTTAATAATGTCGAAGAA |
| | | TGTGGTTAGAGACATGACCCTGCCGTGTACCAGCTGAGAGACTCTAAATCCAGTGACAAGTCTG |
| TC13P4_pool7_30 | 1094 | GAGATCATGTCCTAACCCTGATCCTCTTGTCCCACAGATATCCAGAACCCTCTAAGAAGTTCCTGCTTAAATATGTCGCTGAA |
| | | TGTGGTTAGAGACATGACCCTGCCGTGTACCAGCTGAGAGACTCTAAATCCAGTGACAAGTCTG |
| TC13P4_pool7_31 | 1095 | GAGATCATGTCCTAACCCTGATCCTCTTGTCCCACAGATATCCAGAACCCTCTAAGAAGTTCCTGCTTATAAATGTCGGAGAA |
| | | TGTGGTTAGAGACATGACCCTGCCGTGTACCAGCTGAGAGACTCTAAATCCAGTGACAAGTCTG |
| TC13P4_pool7_32 | 1096 | GAGATCATGTCCTAACCCTGATCCTCTTGTCCCACAGATATCCAGAACCCTCTAAGAAGTTCCTGCTTTAAATTGTCGTTGAAT |
| | | GTGGTTAGAGACATGACCCTGCCGTGTACCAGCTGAGAGACTCTAAATCCAGTGACAAGTCTG |
| TC13P4_pool7_33 | 1097 | GAGATCATGTCCTAACCCTGATCCTCTTGTCCCACAGATATCCAGAACCCTCTAAGAAGTTCCTGCTAATATTTGTCTATGAAT |
| | | GTGGTTAGAGACATGACCCTGCCGTGTACCAGCTGAGAGACTCTAAATCCAGTGACAAGTCTG |
| TC13P4_pool7_34 | 1098 | GAGATCATGTCCTAACCCTGATCCTCTTGTCCCACAGATATCCAGAACCCTCTAAGAAGTTCCTGCTTTATAATGTCTCTGAAT |
| | | GTGGTTAGAGACATGACCCTGCCGTGTACCAGCTGAGAGACTCTAAATCCAGTGACAAGTCTG |
| TC13P4_pool8_1 | 1099 | GAGATCATGTCCTAACCCTGATCCTCTTGTCCCACAGATATCCAGAACCCTCTAAGAAGTTCCTGCTTTAATATGTCTGAGAAT |
| | | GTGGTTAGAGACATGACCCTGCCGTGTACCAGCTGAGAGACTCTAAATCCAGTGACAAGTCTG |
| TC13P4_pool8_2 | 1100 | GAGATCATGTCCTAACCCTGATCCTCTTGTCCCACAGATATCCAGAACCCTCTAAGAAGTTCCTGCTTAATTTTGTCTTTGAAT |
| | | GTGGTTAGAGACATGACCCTGCCGTGTACCAGCTGAGAGACTCTAAATCCAGTGACAAGTCTG |
| TC13P4_pool8_3 | 1101 | GAGATCATGTCCTAACCCTGATCCTCTTGTCCCACAGATATCCAGAACCCTCTAAGAAGTTCCTGCTAATTTTTTTCAAAGAAT |
| | | GTGGTTAGAGACATGACCCTGCCGTGTACCAGCTGAGAGACTCTAAATCCAGTGACAAGTCTG |
| TC13P4_pool8_4 | 1102 | GAGATCATGTCCTAACCCTGATCCTCTTGTCCCACAGATATCCAGAACCCTCTAAGAAGTTCCTGCTTTATATTTTCACAGAAT |
| | | GTGGTTAGAGACATGACCCTGCCGTGTACCAGCTGAGAGACTCTAAATCCAGTGACAAGTCTG |
| TC13P4_pool8_5 | 1103 | GAGATCATGTCCTAACCCTGATCCTCTTGTCCCACAGATATCCAGAACCCTCTAAGAAGTTCCTGCTAATTATTTTCAGTGAAT |
| | | GTGGTTAGAGACATGACCCTGCCGTGTACCAGCTGAGAGACTCTAAATCCAGTGACAAGTCTG |
| TC13P4_pool8_6 | 1104 | GAGATCATGTCCTAACCCTGATCCTCTTGTCCCACAGATATCCAGAACCCTCTAAGAAGTTCCTGCTTTTTAATTTCATAGAAT |
| | | GTGGTTAGAGACATGACCCTGCCGTGTACCAGCTGAGAGACTCTAAATCCAGTGACAAGTCTG |
| TC13P4_pool8_7 | 1105 | GAGATCATGTCCTAACCCTGATCCTCTTGTCCCACAGATATCCAGAACCCTCTAAGAAGTTCCTGCTATTTATTTTCCAAGAAT |
| | | GTGGTTAGAGACATGACCCTGCCGTGTACCAGCTGAGAGACTCTAAATCCAGTGACAAGTCTG |
| TC13P4_pool8_8 | 1106 | GAGATCATGTCCTAACCCTGATCCTCTTGTCCCACAGATATCCAGAACCCTCTAAGAAGTTCCTGCTAAATTATTTCCCTGAAT |
| | | GTGGTTAGAGACATGACCCTGCCGTGTACCAGCTGAGAGACTCTAAATCCAGTGACAAGTCTG |
| TC13P4_pool8_9 | 1107 | GAGATCATGTCCTAACCCTGATCCTCTTGTCCCACAGATATCCAGAACCCTCTAAGAAGTTCCTGCTATAAAATTTCCGAGAA |
| | | TGTGGTTAGAGACATGACCCTGCCGTGTACCAGCTGAGAGACTCTAAATCCAGTGACAAGTCTG |
| TC13P4_pool8_10 | 1108 | GAGATCATGTCCTAACCCTGATCCTCTTGTCCCACAGATATCCAGAACCCTCTAAGAAGTTCCTGCTTATAATTTTCCTTGAAT |
| | | GTGGTTAGAGACATGACCCTGCCGTGTACCAGCTGAGAGACTCTAAATCCAGTGACAAGTCTG |
| TC13P4_pool8_11 | 1109 | GAGATCATGTCCTAACCCTGATCCTCTTGTCCCACAGATATCCAGAACCCTCTAAGAAGTTCCTGCTAAAAATTTTCGATGAAT |
| | | GTGGTTAGAGACATGACCCTGCCGTGTACCAGCTGAGAGACTCTAAATCCAGTGACAAGTCTG |
| TC13P4_pool8_12 | 1110 | GAGATCATGTCCTAACCCTGATCCTCTTGTCCCACAGATATCCAGAACCCTCTAAGAAGTTCCTGCTTTTATTTTTCGCAGAAT |
| | | GTGGTTAGAGACATGACCCTGCCGTGTACCAGCTGAGAGACTCTAAATCCAGTGACAAGTCTG |
| TC13P4_pool8_13 | 1111 | GAGATCATGTCCTAACCCTGATCCTCTTGTCCCACAGATATCCAGAACCCTCTAAGAAGTTCCTGCTAATAAATTTCGGTGAA |
| | | TGTGGTTAGAGACATGACCCTGCCGTGTACCAGCTGAGAGACTCTAAATCCAGTGACAAGTCTG |
| TC13P4_pool8_14 | 1112 | GAGATCATGTCCTAACCCTGATCCTCTTGTCCCACAGATATCCAGAACCCTCTAAGAAGTTCCTGCTTATTTATTTCGTAGAAT |
| | | GTGGTTAGAGACATGACCCTGCCGTGTACCAGCTGAGAGACTCTAAATCCAGTGACAAGTCTG |
| TC13P4_pool8_15 | 1113 | GAGATCATGTCCTAACCCTGATCCTCTTGTCCCACAGATATCCAGAACCCTCTAAGAAGTTCCTGCTTATTAATTTCTATGAAT |
| | | GTGGTTAGAGACATGACCCTGCCGTGTACCAGCTGAGAGACTCTAAATCCAGTGACAAGTCTG |
| TC13P4_pool8_16 | 1114 | GAGATCATGTCCTAACCCTGATCCTCTTGTCCCACAGATATCCAGAACCCTCTAAGAAGTTCCTGCTTTTATATTTCTCTGAAT |
| | | GTGGTTAGAGACATGACCCTGCCGTGTACCAGCTGAGAGACTCTAAATCCAGTGACAAGTCTG |
| TC13P4_pool8_17 | 1115 | GAGATCATGTCCTAACCCTGATCCTCTTGTCCCACAGATATCCAGAACCCTCTAAGAAGTTCCTGCTTATTATTTTCTGAGAAT |
| | | GTGGTTAGAGACATGACCCTGCCGTGTACCAGCTGAGAGACTCTAAATCCAGTGACAAGTCTG |
| TC13P4_pool8_18 | 1116 | GAGATCATGTCCTAACCCTGATCCTCTTGTCCCACAGATATCCAGAACCCTCTAAGAAGTTCCTGCTATATATTTTCTTTGAAT |
| | | GTGGTTAGAGACATGACCCTGCCGTGTACCAGCTGAGAGACTCTAAATCCAGTGACAAGTCTG |

TABLE 18
List of disease due to a deleterious allele (target gene) that can be addressed by the invention

| Disease name | Target Gene |
|---|---|
| Hereditary amyloid angiopathies | APP |
| Marfan Syndrome | FBN1 |
| Neurofibromatosis type 1 | NF1 |
| Familial adenomatous polyposis | APC |
| Tuberous sclerosis complex (TSC) | TSC1 or TSC2 |
| Brugada syndrome | SCN5A |
| APDS2 and SHORT Syndrome | PIK3R1 |
| Vascular Ehlers-Danlos syndrome | COL3A1 |
| APDS1 | PIK3CD |
| congenital neutropenia | SRP54 |
| autosomal dominant hyper-IgE syndrome (AD-HIES) | stat3 |
| hepatic disease with severe immunodeficiency | NFKBIA |
| Laron syndrome | GHR, growth hormone recepto [symbol] |
| Pituitary hormone deficiency | POU1F1, POU domain, Class1 |
| Growth hormone deficiency | GH1 |
| thyroid hormone receptor β Thyroid hormone resistance | THRB |
| gonadotropin-releasing hormone receptor Hypogonadotropic hypogonadism | GNRHR, gonadotropin-releasing hormone receptor |
| UDP glycosyltransferase 1 superfamily Gilbert syndrome | GLI2, GLI, |
| UDP glycosyltransferase 1 superfamily Gilbert syndrome | UGT1A1 |
| Li-Fraumeni syndrome | TP53, tumor protein p53 |
| Rubinstein-Taybi syndrome | CREBBP |
| Wilms tumor 1 Denys-Drash syndrome | WT1 |

[symbol]

indicates data missing or illegible when filed

1-35. (canceled)

36. A method for designing and producing a TALE base editor heterodimer to convert a specific C into A, and/or its complementary G position into T, in a double stranded nucleic acid sequence, said method comprising the step of
i) identifying in said nucleic acid sequence a target sequence selected from:
5′-T₀-N_{left}-N_{y}-RTC-N_{X}-N_{right}-A₀-3′;
and
5′-T₀-N_{left}-Nₓ-GAY-N_{y}-N_{right}-A₀-3′;
wherein
N can be adenine (A), thymine (T), cytosine (C). or guanine (G),
R can be G or A,
Y can be C or T,
N_{left} can be a polynucleotide sequence from 9 to 20 nucleotides, where each individual nucleotide can be A, T, C or G,
N_{right} can be a polynucleotide sequence from 9 to 20 nucleotides, where each individual nucleotide can be A, T, C or G,
G being the complementary base of C,
x=2 to 6; and
y=6 to 10, with x+y≥11;
ii) synthetizing polynucleotide sequences encoding left and right TALE binding polypeptides that bind the N_{left} and N_{right} polynucleotide sequences, respectively.
iii) fusing said polynucleotide sequence encoding left TALE binding polypeptide to a polynucleotide encoding a N-terminal split DddAtox; and
iv) fusing said polynucleotide sequence encoding right TALE binding polypeptide to a polynucleotide encoding a C terminal split DddAtox fusing a polynucleotide sequence encoding UGI (Uracil glycosylase inhibitor) to at least one polynucleotide sequence encoding said polynucleotide sequence resulting from ii) and iii).

37. The method of claim 36, wherein said left and right TALE binding polypeptides comprise about 11 or 40 amino acids from SEQ ID NO: 270.

38. The method of claim 36, wherein x is 3, 4, or 5.

39. The method of claim 36, wherein the sequence(s) of said N terminal split DddAtox and/or of said C terminal split DddAtox comprise(s) at least one mutation that decreases the affinity of said split DddAtox for each other.

40. The method of claim 39, wherein said mutation is introduced in said C terminal split DddAtox of SEQ ID NO:29.

41. The method of claim 36, wherein said left and right TALE binding polypeptides comprise AvrBs3-like repeats of canonical sequence selected from SEQ ID NO:12 to 15.

42. The method of claim 36, wherein said left and right TALE binding polypeptides comprise AvrBs3-like repeats comprising D (aspartic acid) residues at positions 4 and 32 with respect to any of the canonical sequence of AvrBs3.

43. The method of claim 42, wherein at least one of said AvrBs3-like repeats comprises one polypeptide sequence selected from the group consisting of:
```
  (SEQ ID NO: 5)
LTPDQVVAIASX₁₂X₁₃GGKQALETVQRLLPVLCQDHG,
  (SEQ ID NO: 6)
LTPDQVVAIASX₁₂X₁₃GGKQALETVQALLPVLCQDHG
  (SEQ ID NO: 7)
LTPDQVVAIASX₁₂X₁₃GGKQALETVQQLLPVLCQDHG,
or
  (SEQ ID NO: 8)
LTPDQLVAIASX₁₂X₁₃GGKQALETVQRLLPVLCQDHG,
  (SEQ ID NO: 9)
LTPDQMVAIASX₁₂X₁₃GGKQALETVQRLLPVLCQDHG,
  (SEQ ID NO: 10)
LTPDQVVAIASX₁₂X₁₃GGKQALETVQRLLPVLCQDQG,
and
  (SEQ ID NO: 11)
LTLDQVVAIASX₁₂X₁₃GGKQALETVQRLLPVLCQDHG,
```
wherein X₁₂X₁₃ is an amino acid forming a variable di-residue.

44. The method of claim 36, wherein said C-terminal domain of said TALE binding polypeptide(s) consists of a polypeptide sequence from 40 to 80 residues having at least 85% identity with:
```
  (SEQ ID NO: 2)
SIVAQLSRPDP;
  (SEQ ID NO: 3)
SIVAQLSRPDPALAALTNDHLVALACLGGRPALDAVX₁X₂GL;
(SEQ ID NO: 4)
SIVAQLSRPDPALAALTNDHLVALACLGGRPALDAVX₁X₂GLP
HAPALIX₃RT,
```
or
wherein X₁, X₂, and X₃, are K (Lysine), H (histidine) or a R (arginine) residue.

45. The method of claim 36, further comprising the step of expressing in a cell the polynucleotides obtained in step iv) encoding the TALE base editor heterodimer to introduce a mutation into an immune cell.

46. A method for introducing a mutation into the genome of a cell, comprising the step of introducing or expressing into the cell a TALE base editor consisting of a heterodimeric fusion of a left and right TALE binding polypeptides having a C-terminal domain of about 1 to 50 amino acids, with respectively a C terminal and N terminal split DddATox, wherein said heterodimeric TALE base editor binds a genomic sequence selected from:
5′-T₀-N_{left}-N_{y}-RTC-N_{X}-N_{right}-A₀-3′;
and
5′-T₀-N_{left}-Nₓ-GAY-N_{y}-N_{right}-A₀-3′;
wherein
N can be adenine (A), thymine (T), cytosine (C). or guanine (G),
R can be G or A,
Y can be C or T,
N_{left} can be a polynucleotide sequence from 9 to 20 nucleotides, where each individual nucleotide can be A, T, C or G,
N_{right} can be a polynucleotide sequence from 9 to 20 nucleotides, where each individual nucleotide can be A, T, C or G,
G being the complementary base of C,
x=2 to 6; and
y=6 to 10, with x+y≥11.

47. The method of claim 46, wherein said left and right TALE binding polypeptides have a C-terminal domain of about 11 or 40 amino acids.

48. The method of claim 46, wherein x is 3, 4, or 5.

49. The method of claim 46, wherein said cell is a hematopoietic stem cell, an immune cell, a primary cell, a T-cell, or NK cell.

50. The method of claim 46, wherein said immune cell is endowed with a chimeric antigen receptor (CAR) or a recombinant TCR.

51. The method of claim 46, wherein said TALE base editor binds a genomic sequence in a TRAC gene selected from any one of SEQ ID NO:366 to SEQ ID NO:407.

52. The method of claim 46, wherein said TALE base editor binds a genomic sequence in a CD52 gene selected from any one of SEQ ID NO:408 to SEQ ID NO:422.

53. The method of claim 46, wherein said TALE base editor binds a genomic sequence in a PD1 gene selected from any one of SEQ ID NO:423 to SEQ ID NO:466.

54. The method of claim 46, wherein said TALE base editor binds a genomic sequence in a B2M gene selected from any one of SEQ ID NO:467 to SEQ ID NO:501.

55. The method of claim 46, wherein said TALE base editor binds a genomic sequence in an ApoC3 selected from any one of SEQ ID NO:502 to SEQ ID NO:523.
